SEQUENCE LISTING (2036 sequences)

SEQ ID NO: 1 - protein, 14 aa, Artificial sequence
  TCR-CDR3 peptide sequence
CASSLGGNQDTQYF

SEQ ID NO: 2 - protein, 13 aa, Artificial sequence
  TCR-CDR3 peptide sequence
CASSGTGQDTQYF

SEQ ID NO: 3 - protein, 13 aa, Artificial sequence
  TCR-CDR3 peptide sequence
CASSGTGEDTQYF

SEQ ID NO: 4 - protein, 13 aa, Artificial sequence
  TCR-CDR3 peptide sequence
CASSGLGEDTQYF

SEQ ID NO: 5 - protein, 10 aa, Artificial sequence
  TCR-CDR3 peptide sequence
CASGGYEQYF

SEQ ID NO: 6 - protein, 13 aa, Artificial sequence
  TCR-CDR3 peptide sequence
CASSPGGSYEQYF

SEQ ID NO: 7 - protein, 11 aa, Artificial sequence
  TCR-CDR3 peptide sequence
CASSSRYEQYF

SEQ ID NO: 8 - protein, 13 aa, Artificial sequence
  TCR-CDR3 peptide sequence
CASSGTGKDTQYF

SEQ ID NO: 9 - protein, 13 aa, Artificial sequence
  TCR-CDR3 peptide sequence
CASSFGVSYEQYF

SEQ ID NO: 10 - protein, 12 aa, Artificial sequence
  TCR-CDR3 peptide sequence
CASSRGSYEQYF

SEQ ID NO: 11 - protein, 13 aa, Artificial sequence
  TCR-CDR3 peptide sequence
CASSPGTGVEQYF

SEQ ID NO: 12 - protein, 13 aa, Artificial sequence
  TCR-CDR3 peptide sequence
CASSFGTGYEQYF

SEQ ID NO: 13 - protein, 12 aa, Artificial sequence
  TCR-CDR3 peptide sequence
CASSGGAYEQYF

SEQ ID NO: 14 - protein, 13 aa, Artificial sequence
  TCR-CDR3 peptide sequence
CASSLGVGDTQYF

SEQ ID NO: 15 - protein, 13 aa, Artificial sequence
  TCR-CDR3 peptide sequence
CASTGTGQDTQYF

SEQ ID NO: 16 - protein, 13 aa, Artificial sequence
  TCR-CDR3 peptide sequence
CASSGRGQDTQYF

SEQ ID NO: 17 - protein, 11 aa, Artificial sequence
  TCR-CDR3 peptide sequence
CASGGAYEQYF

SEQ ID NO: 18 - protein, 13 aa, Artificial sequence
  TCR-CDR3 peptide sequence
CASSFVGSYEQYF

SEQ ID NO: 19 - protein, 12 aa, Artificial sequence
  TCR-CDR3 peptide sequence
CASSRRPYEQYF

SEQ ID NO: 20 - protein, 13 aa, Artificial sequence
  TCR-CDR3 peptide sequence
CASSLGGQNTLYF

SEQ ID NO: 21 - protein, 12 aa, Artificial sequence
  TCR-CDR3 peptide sequence
CASSLGNSDYTF

SEQ ID NO: 22 - protein, 12 aa, Artificial sequence
  TCR-CDR3 peptide sequence
CASSSANSDYTF

SEQ ID NO: 23 - protein, 12 aa, Artificial sequence
  TCR-CDR3 peptide sequence
CASSSGNSDYTF

SEQ ID NO: 24 - protein, 13 aa, Artificial sequence
  TCR-CDR3 peptide sequence
CASSGTANTEVFF

SEQ ID NO: 25 - protein, 14 aa, Artificial sequence
  TCR-CDR3 peptide sequence
CASSGQGNYAEQFF

SEQ ID NO: 26 - protein, 12 aa, Artificial sequence
  TCR-CDR3 peptide sequence
CASGDWGYEQYF

SEQ ID NO: 27 - protein, 14 aa, Artificial sequence
  TCR-CDR3 peptide sequence
CASGDAGGSYEQYF

SEQ ID NO: 28 - protein, 13 aa, Artificial sequence
  TCR-CDR3 peptide sequence
CASSDGANTEVFF

SEQ ID NO: 29 - protein, 12 aa, Artificial sequence
  TCR-CDR3 peptide sequence
CASSHSGNTLYF

SEQ ID NO: 30 - protein, 13 aa, Artificial sequence
  TCR-CDR3 peptide sequence
CASSGTDQDTQYF

SEQ ID NO: 31 - protein, 14 aa, Artificial sequence
  TCR-CDR3 peptide sequence
CASSPGQSNERLFF

SEQ ID NO: 32 - protein, 14 aa, Artificial sequence
  TCR-CDR3 peptide sequence
CASSRTANTGQLYF

SEQ ID NO: 33 - protein, 13 aa, Artificial sequence
  TCR-CDR3 peptide sequence
CASSDSANTEVFF

SEQ ID NO: 34 - protein, 13 aa, Artificial sequence
  TCR-CDR3 peptide sequence
CASSLEGDTEVFF

SEQ ID NO: 35 - protein, 13 aa, Artificial sequence
  TCR-CDR3 peptide sequence
CASSLEGEDTQYF

SEQ ID NO: 36 - protein, 13 aa, Artificial sequence
  TCR-CDR3 peptide sequence
CASSPGQQDTQYF

SEQ ID NO: 37 - protein, 11 aa, Artificial sequence
  TCR-CDR3 peptide sequence
CASSFQDTQYF

SEQ ID NO: 38 - protein, 12 aa, Artificial sequence
  TCR-CDR3 peptide sequence
CASSRQQDTQYF

SEQ ID NO: 39 - protein, 13 aa, Artificial sequence
  TCR-CDR3 peptide sequence
CASSRDSQDTQYF

SEQ ID NO: 40 - protein, 12 aa, Artificial sequence
  TCR-CDR3 peptide sequence
CASSLQGYEQYF

SEQ ID NO: 41 - protein, 12 aa, Artificial sequence
  TCR-CDR3 peptide sequence
CASSDSSYEQYF

SEQ ID NO: 42 - protein, 13 aa, Artificial sequence
  TCR-CDR3 peptide sequence
CASSLGSSYEQYF

SEQ ID NO: 43 - protein, 11 aa, Artificial sequence
  TCR-CDR3 peptide sequence
CASGDGDTQYF

SEQ ID NO: 44 - protein, 10 aa, Artificial sequence
  TCR-CDR3 peptide sequence
CASGDYEQYF

SEQ ID NO: 45 - protein, 11 aa, Artificial sequence
  TCR-CDR3 peptide sequence
CASSLEDTQYF

SEQ ID NO: 46 - protein, 12 aa, Artificial sequence
  TCR-CDR3 peptide sequence
CASSLEGDEQYF

SEQ ID NO: 47 - protein, 11 aa, Artificial sequence
  TCR-CDR3 peptide sequence
CASSLGYEQYF

SEQ ID NO: 48 - protein, 12 aa, Artificial sequence
  TCR-CDR3 peptide sequence
CASSLGQYEQYF

SEQ ID NO: 49 - protein, 13 aa, Artificial sequence
  TCR-CDR3 peptide sequence
CASSVDGSYEQYF

SEQ ID NO: 50 - protein, 11 aa, Artificial sequence
  TCR-CDR3 peptide sequence
CASSPQDTQYF

SEQ ID NO: 51 - protein, 12 aa, Artificial sequence
  TCR-CDR3 peptide sequence
CASSLDNYEQYF

SEQ ID NO: 52 - protein, 12 aa, Artificial sequence
  TCR-CDR3 peptide sequence
CASSLEGYEQYF

SEQ ID NO: 53 - protein, 10 aa, Artificial sequence
  TCR-CDR3 peptide sequence
CASSLDEQYF

SEQ ID NO: 54 - protein, 14 aa, Artificial sequence
  TCR-CDR3 peptide sequence
CASSLEGNQDTQYF

SEQ ID NO: 55 - protein, 12 aa, Artificial sequence
  TCR-CDR3 peptide sequence
CASSLEGDTQYF

SEQ ID NO: 56 - protein, 11 aa, Artificial sequence
  TCR-CDR3 peptide sequence
CASSLDYEQYF

SEQ ID NO: 57 - protein, 11 aa, Artificial sequence
  TCR-CDR3 peptide sequence
CASSLGDTQYF

SEQ ID NO: 58 - protein, 10 aa, Artificial sequence
  TCR-CDR3 peptide sequence
CASSLGEQYF

SEQ ID NO: 59 - protein, 10 aa, Artificial sequence
  TCR-CDR3 peptide sequence
CASSQDTQYF

SEQ ID NO: 60 - protein, 12 aa, Artificial sequence
  TCR-CDR3 peptide sequence
CASSLDRYEQYF

SEQ ID NO: 61 - protein, 13 aa, Artificial sequence
  TCR-CDR3 peptide sequence
CASSGTGGYEQYF

SEQ ID NO: 62 - protein, 10 aa, Artificial sequence
  TCR-CDR3 peptide sequence
CASSSYEQYF

SEQ ID NO: 63 - protein, 12 aa, Artificial sequence
  TCR-CDR3 peptide sequence
CASSSGQYEQYF

SEQ ID NO: 64 - protein, 13 aa, Artificial sequence
  TCR-CDR3 peptide sequence
CASSLLGGAEQFF

SEQ ID NO: 65 - protein, 12 aa, Artificial sequence
  TCR-CDR3 peptide sequence
CASSLDRDEQYF

SEQ ID NO: 66 - protein, 12 aa, Artificial sequence
  TCR-CDR3 peptide sequence
CASSLDQDTQYF

SEQ ID NO: 67 - protein, 14 aa, Artificial sequence
  TCR-CDR3 peptide sequence
CASSLEGSSYEQYF

SEQ ID NO: 68 - protein, 12 aa, Artificial sequence
  TCR-CDR3 peptide sequence
CASSLGGYEQYF

SEQ ID NO: 69 - protein, 11 aa, Artificial sequence
  TCR-CDR3 peptide sequence
CASSLGAEQFF

SEQ ID NO: 70 - protein, 12 aa, Artificial sequence
  TCR-CDR3 peptide sequence
CASSLSGYEQYF

SEQ ID NO: 71 - protein, 11 aa, Artificial sequence
  TCR-CDR3 peptide sequence
CASSSSYEQYF

SEQ ID NO: 72 - protein, 12 aa, Artificial sequence
  TCR-CDR3 peptide sequence
CASSLGQDTQYF

SEQ ID NO: 73 - protein, 13 aa, Artificial sequence
  TCR-CDR3 peptide sequence
CASSLGGQDTQYF

SEQ ID NO: 74 - protein, 12 aa, Artificial sequence
  TCR-CDR3 peptide sequence
CASSFNQDTQYF

SEQ ID NO: 75 - protein, 10 aa, Artificial sequence
  TCR-CDR3 peptide sequence
CASGQDTQYF

SEQ ID NO: 76 - protein, 13 aa, Artificial sequence
  TCR-CDR3 peptide sequence
CASSRDWGYEQYF

SEQ ID NO: 77 - protein, 12 aa, Artificial sequence
  TCR-CDR3 peptide sequence
CASSLDSYEQYF

SEQ ID NO: 78 - protein, 11 aa, Artificial sequence
  TCR-CDR3 peptide sequence
CASSRQYEQYF

SEQ ID NO: 79 - protein, 11 aa, Artificial sequence
  TCR-CDR3 peptide sequence
CASSLKDTQYF

SEQ ID NO: 80 - protein, 13 aa, Artificial sequence
  TCR-CDR3 peptide sequence
CASSLGQNTEVFF

SEQ ID NO: 81 - protein, 13 aa, Artificial sequence
  TCR-CDR3 peptide sequence
CASSFGTGDEQYF

SEQ ID NO: 82 - protein, 12 aa, Artificial sequence
  TCR-CDR3 peptide sequence
CASSLGEDTQYF

SEQ ID NO: 83 - protein, 13 aa, Artificial sequence
  TCR-CDR3 peptide sequence
CASSRQNQDTQYF

SEQ ID NO: 84 - protein, 12 aa, Artificial sequence
  TCR-CDR3 peptide sequence
CASSPSSYEQYF

SEQ ID NO: 85 - protein, 12 aa, Artificial sequence
  TCR-CDR3 peptide sequence
CASSPDSYEQYF

SEQ ID NO: 86 - protein, 12 aa, Artificial sequence
  TCR-CDR3 peptide sequence
CASSRDNYEQYF

SEQ ID NO: 87 - protein, 12 aa, Artificial sequence
  TCR-CDR3 peptide sequence
CASSRDSYEQYF

SEQ ID NO: 88 - protein, 12 aa, Artificial sequence
  TCR-CDR3 peptide sequence
CASSLDRVEQYF

SEQ ID NO: 89 - protein, 11 aa, Artificial sequence
  TCR-CDR3 peptide sequence
CASSLYAEQFF

SEQ ID NO: 90 - protein, 11 aa, Artificial sequence
  TCR-CDR3 peptide sequence
CASSLDAEQFF

SEQ ID NO: 91 - protein, 11 aa, Artificial sequence
  TCR-CDR3 peptide sequence
CASSDSYEQYF

SEQ ID NO: 92 - protein, 11 aa, Artificial sequence
  TCR-CDR3 peptide sequence
CASSFGTEVFF

SEQ ID NO: 93 - protein, 12 aa, Artificial sequence
  TCR-CDR3 peptide sequence
CASSPDNYEQYF

SEQ ID NO: 94 - protein, 12 aa, Artificial sequence
  TCR-CDR3 peptide sequence
CASSPGQYEQYF

SEQ ID NO: 95 - protein, 11 aa, Artificial sequence
  TCR-CDR3 peptide sequence
CASSLQDTQYF

SEQ ID NO: 96 - protein, 11 aa, Artificial sequence
  TCR-CDR3 peptide sequence
CASSLRDTQYF

SEQ ID NO: 97 - protein, 11 aa, Artificial sequence
  TCR-CDR3 peptide sequence
CASSLGDEQYF

SEQ ID NO: 98 - protein, 11 aa, Artificial sequence
  TCR-CDR3 peptide sequence
CASSLVAEQFF

SEQ ID NO: 99 - protein, 11 aa, Artificial sequence
  TCR-CDR3 peptide sequence
CASSFSYEQYF

SEQ ID NO: 100 - protein, 11 aa, Artificial sequence
  TCR-CDR3 peptide sequence
CASSLSYEQYF

SEQ ID NO: 101 - protein, 11 aa, Artificial sequence
  TCR-CDR3 peptide sequence
CASSDQDTQYF

SEQ ID NO: 102 - protein, 12 aa, Artificial sequence
  TCR-CDR3 peptide sequence
CASSDAGDTQYF

SEQ ID NO: 103 - protein, 11 aa, Artificial sequence
  TCR-CDR3 peptide sequence
CASGDSYEQYF

SEQ ID NO: 104 - protein, 10 aa, Artificial sequence
  TCR-CDR3 peptide sequence
CASSLDTQYF

SEQ ID NO: 105 - protein, 11 aa, Artificial sequence
  TCR-CDR3 peptide sequence
CASSFYAEQFF

SEQ ID NO: 106 - protein, 9 aa, Artificial sequence
  TCR-CDR3 peptide sequence
CASGDEQYF

SEQ ID NO: 107 - protein, 11 aa, Artificial sequence
  TCR-CDR3 peptide sequence
CASGDAYEQYF

SEQ ID NO: 108 - protein, 10 aa, Artificial sequence
  TCR-CDR3 peptide sequence
CASSLYEQYF

SEQ ID NO: 109 - protein, 13 aa, Artificial sequence
  TCR-CDR3 peptide sequence
CASSRDSSYEQYF

SEQ ID NO: 110 - protein, 11 aa, Artificial sequence
  TCR-CDR3 peptide sequence
CASSIRDTQYF

SEQ ID NO: 111 - protein, 12 aa, Artificial sequence
  TCR-CDR3 peptide sequence
CASSRTGYEQYF

SEQ ID NO: 112 - protein, 12 aa, Artificial sequence
  TCR-CDR3 peptide sequence
CASSDAGYEQYF

SEQ ID NO: 113 - protein, 11 aa, Artificial sequence
  TCR-CDR3 peptide sequence
CASSQQDTQYF

SEQ ID NO: 114 - protein, 12 aa, Artificial sequence
  TCR-CDR3 peptide sequence
CASSGQQDTQYF

SEQ ID NO: 115 - protein, 10 aa, Artificial sequence
  TCR-CDR3 peptide sequence
CASGEDTQYF

SEQ ID NO: 116 - protein, 11 aa, Artificial sequence
  TCR-CDR3 peptide sequence
CASSRQDTQYF

SEQ ID NO: 117 - protein, 13 aa, Artificial sequence
  TCR-CDR3 peptide sequence
CASSLGQQDTQYF

SEQ ID NO: 118 - protein, 10 aa, Artificial sequence
  TCR-CDR3 peptide sequence
CASSGGEQYF

SEQ ID NO: 119 - protein, 11 aa, Artificial sequence
  TCR-CDR3 peptide sequence
CASSYQDTQYF

SEQ ID NO: 120 - protein, 11 aa, Artificial sequence
  TCR-CDR3 peptide sequence
CASSLQYEQYF

SEQ ID NO: 121 - protein, 11 aa, Artificial sequence
  TCR-CDR3 peptide sequence
CASSSQDTQYF

SEQ ID NO: 122 - protein, 13 aa, Artificial sequence
  TCR-CDR3 peptide sequence
CASSQDRDTEVFF

SEQ ID NO: 123 - protein, 11 aa, Artificial sequence
  TCR-CDR3 peptide sequence
CASSNQDTQYF

SEQ ID NO: 124 - protein, 12 aa, Artificial sequence
  TCR-CDR3 peptide sequence
CASSSSSYEQYF

SEQ ID NO: 125 - protein, 12 aa, Artificial sequence
  TCR-CDR3 peptide sequence
CASSLEGAEQFF

SEQ ID NO: 126 - protein, 12 aa, Artificial sequence
  TCR-CDR3 peptide sequence
CASSFGQYEQYF

SEQ ID NO: 127 - protein, 12 aa, Artificial sequence
  TCR-CDR3 peptide sequence
CASSLTGDEQYF

SEQ ID NO: 128 - protein, 12 aa, Artificial sequence
  TCR-CDR3 peptide sequence
CASSRQGYEQYF

SEQ ID NO: 129 - protein, 12 aa, Artificial sequence
  TCR-CDR3 peptide sequence
CASSSTGYEQYF

SEQ ID NO: 130 - protein, 12 aa, Artificial sequence
  TCR-CDR3 peptide sequence
CASSLDGYEQYF

SEQ ID NO: 131 - protein, 12 aa, Artificial sequence
  TCR-CDR3 peptide sequence
CASSSNQDTQYF

SEQ ID NO: 132 - protein, 12 aa, Artificial sequence
  TCR-CDR3 peptide sequence
CASSPTGDEQYF

SEQ ID NO: 133 - protein, 11 aa, Artificial sequence
  TCR-CDR3 peptide sequence
CASSLSAEQFF

SEQ ID NO: 134 - protein, 12 aa, Artificial sequence
  TCR-CDR3 peptide sequence
CASSLDRGEQYF

SEQ ID NO: 135 - protein, 12 aa, Artificial sequence
  TCR-CDR3 peptide sequence
CASSLGYAEQFF

SEQ ID NO: 136 - protein, 12 aa, Artificial sequence
  TCR-CDR3 peptide sequence
CASSSQGYEQYF

SEQ ID NO: 137 - protein, 11 aa, Artificial sequence
  TCR-CDR3 peptide sequence
CASSPSYEQYF

SEQ ID NO: 138 - protein, 12 aa, Artificial sequence
  TCR-CDR3 peptide sequence
CASSPDRYEQYF

SEQ ID NO: 139 - protein, 12 aa, Artificial sequence
  TCR-CDR3 peptide sequence
CASSPNQDTQYF

SEQ ID NO: 140 - protein, 12 aa, Artificial sequence
  TCR-CDR3 peptide sequence
CASSLSSYEQYF

SEQ ID NO: 141 - protein, 12 aa, Artificial sequence
  TCR-CDR3 peptide sequence
CASSLAGYEQYF

SEQ ID NO: 142 - protein, 13 aa, Artificial sequence
  TCR-CDR3 peptide sequence
CASSLTGGYEQYF

SEQ ID NO: 143 - protein, 12 aa, Artificial sequence
  TCR-CDR3 peptide sequence
CASSLDTYEQYF

SEQ ID NO: 144 - protein, 14 aa, Artificial sequence
  TCR-CDR3 peptide sequence
CASSLGGSSYEQYF

SEQ ID NO: 145 - protein, 9 aa, Artificial sequence
  TCR-CDR3 peptide sequence
CASGYEQYF

SEQ ID NO: 146 - protein, 11 aa, Artificial sequence
  TCR-CDR3 peptide sequence
CASGDADTQYF

SEQ ID NO: 147 - protein, 12 aa, Artificial sequence
  TCR-CDR3 peptide sequence
CASSLVGYEQYF

SEQ ID NO: 148 - protein, 11 aa, Artificial sequence
  TCR-CDR3 peptide sequence
CASGDEDTQYF

SEQ ID NO: 149 - protein, 12 aa, Artificial sequence
  TCR-CDR3 peptide sequence
CASSQGQYEQYF

SEQ ID NO: 150 - protein, 13 aa, Artificial sequence
  TCR-CDR3 peptide sequence
CASSLDSSYEQYF

SEQ ID NO: 151 - protein, 12 aa, Artificial sequence
  TCR-CDR3 peptide sequence
CASSPTGYEQYF

SEQ ID NO: 152 - protein, 10 aa, Artificial sequence
  TCR-CDR3 peptide sequence
CASSQYEQYF

SEQ ID NO: 153 - protein, 13 aa, Artificial sequence
  TCR-CDR3 peptide sequence
CASSRQGQDTQYF

SEQ ID NO: 154 - protein, 12 aa, Artificial sequence
  TCR-CDR3 peptide sequence
CASSLQGAEQFF

SEQ ID NO: 155 - protein, 11 aa, Artificial sequence
  TCR-CDR3 peptide sequence
CASSDNYEQYF

SEQ ID NO: 156 - protein, 12 aa, Artificial sequence
  TCR-CDR3 peptide sequence
CASSDRGDTQYF

SEQ ID NO: 157 - protein, 10 aa, Artificial sequence
  TCR-CDR3 peptide sequence
CASSDAEQFF

SEQ ID NO: 158 - protein, 11 aa, Artificial sequence
  TCR-CDR3 peptide sequence
CASSGQYEQYF

SEQ ID NO: 159 - protein, 13 aa, Artificial sequence
  TCR-CDR3 peptide sequence
CASSSGGQDTQYF

SEQ ID NO: 160 - protein, 12 aa, Artificial sequence
  TCR-CDR3 peptide sequence
CASSLGGAEQFF

SEQ ID NO: 161 - protein, 11 aa, Artificial sequence
  TCR-CDR3 peptide sequence
CASSPDAEQFF

SEQ ID NO: 162 - protein, 13 aa, Artificial sequence
  TCR-CDR3 peptide sequence
CASSLGGSYEQYF

SEQ ID NO: 163 - protein, 12 aa, Artificial sequence
  TCR-CDR3 peptide sequence
CASSLAGDEQYF

SEQ ID NO: 164 - protein, 13 aa, Artificial sequence
  TCR-CDR3 peptide sequence
CASSLSGGYEQYF

SEQ ID NO: 165 - protein, 12 aa, Artificial sequence
  TCR-CDR3 peptide sequence
CASSYNQDTQYF

SEQ ID NO: 166 - protein, 11 aa, Artificial sequence
  TCR-CDR3 peptide sequence
CASSLEGEQYF

SEQ ID NO: 167 - protein, 13 aa, Artificial sequence
  TCR-CDR3 peptide sequence
CASSRDRGYEQYF

SEQ ID NO: 168 - protein, 11 aa, Artificial sequence
  TCR-CDR3 peptide sequence
CASSLGGEQYF

SEQ ID NO: 169 - protein, 10 aa, Artificial sequence
  TCR-CDR3 peptide sequence
CASSLEEQYF

SEQ ID NO: 170 - protein, 10 aa, Artificial sequence
  TCR-CDR3 peptide sequence
CASGDDEQYF

SEQ ID NO: 171 - protein, 13 aa, Artificial sequence
  TCR-CDR3 peptide sequence
CASSRLPSYEQYF

SEQ ID NO: 172 - protein, 14 aa, Artificial sequence
  TCR-CDR3 peptide sequence
CASSLAGGQDTQYF

SEQ ID NO: 173 - protein, 14 aa, Artificial sequence
  TCR-CDR3 peptide sequence
CASSRTGGQDTQYF

SEQ ID NO: 174 - protein, 13 aa, Artificial sequence
  TCR-CDR3 peptide sequence
CASSLEGQDTQYF

SEQ ID NO: 175 - protein, 12 aa, Artificial sequence
  TCR-CDR3 peptide sequence
CASSLVGDEQYF

SEQ ID NO: 176 - protein, 12 aa, Artificial sequence
  TCR-CDR3 peptide sequence
CASSPQGYEQYF

SEQ ID NO: 177 - protein, 12 aa, Artificial sequence
  TCR-CDR3 peptide sequence
CASSSDSYEQYF

SEQ ID NO: 178 - protein, 13 aa, Artificial sequence
  TCR-CDR3 peptide sequence
CASSRTGEDTQYF

SEQ ID NO: 179 - protein, 13 aa, Artificial sequence
  TCR-CDR3 peptide sequence
CASSRTGGYEQYF

SEQ ID NO: 180 - protein, 14 aa, Artificial sequence
  TCR-CDR3 peptide sequence
CASSLQGSSYEQYF

SEQ ID NO: 181 - protein, 13 aa, Artificial sequence
  TCR-CDR3 peptide sequence
CASSLTGNTEVFF

SEQ ID NO: 182 - protein, 12 aa, Artificial sequence
  TCR-CDR3 peptide sequence
CASSLGGDTQYF

SEQ ID NO: 183 - protein, 13 aa, Artificial sequence
  TCR-CDR3 peptide sequence
CASSLDWGYEQYF

SEQ ID NO: 184 - protein, 14 aa, Artificial sequence
  TCR-CDR3 peptide sequence
CASSPGQSSYEQYF

SEQ ID NO: 185 - protein, 11 aa, Artificial sequence
  TCR-CDR3 peptide sequence
CASSYSYEQYF

SEQ ID NO: 186 - protein, 10 aa, Artificial sequence
  TCR-CDR3 peptide sequence
CASSLVEQYF

SEQ ID NO: 187 - protein, 14 aa, Artificial sequence
  TCR-CDR3 peptide sequence
CASSLDRGNTEVFF

SEQ ID NO: 188 - protein, 12 aa, Artificial sequence
  TCR-CDR3 peptide sequence
CASSLLGYEQYF

SEQ ID NO: 189 - protein, 12 aa, Artificial sequence
  TCR-CDR3 peptide sequence
CASSQSSYEQYF

SEQ ID NO: 190 - protein, 11 aa, Artificial sequence
  TCR-CDR3 peptide sequence
CASSEGDTQYF

SEQ ID NO: 191 - protein, 11 aa, Artificial sequence
  TCR-CDR3 peptide sequence
CASSDRDTQYF

SEQ ID NO: 192 - protein, 11 aa, Artificial sequence
  TCR-CDR3 peptide sequence
CASSDGDTQYF

SEQ ID NO: 193 - protein, 10 aa, Artificial sequence
  TCR-CDR3 peptide sequence
CASSFYEQYF

SEQ ID NO: 194 - protein, 9 aa, Artificial sequence
  TCR-CDR3 peptide sequence
CASSYEQYF

SEQ ID NO: 195 - protein, 13 aa, Artificial sequence
  TCR-CDR3 peptide sequence
CASSRQSSYEQYF

SEQ ID NO: 196 - protein, 13 aa, Artificial sequence
  TCR-CDR3 peptide sequence
CASSRDREDTQYF

SEQ ID NO: 197 - protein, 10 aa, Artificial sequence
  TCR-CDR3 peptide sequence
CASSRYEQYF

SEQ ID NO: 198 - protein, 12 aa, Artificial sequence
  TCR-CDR3 peptide sequence
CASSPQGTEVFF

SEQ ID NO: 199 - protein, 14 aa, Artificial sequence
  TCR-CDR3 peptide sequence
CASSPGQGQDTQYF

SEQ ID NO: 200 - protein, 12 aa, Artificial sequence
  TCR-CDR3 peptide sequence
CASSGDSYEQYF

SEQ ID NO: 201 - protein, 11 aa, Artificial sequence
  TCR-CDR3 peptide sequence
CASGDFYEQYF

SEQ ID NO: 202 - protein, 10 aa, Artificial sequence
  TCR-CDR3 peptide sequence
CASSQGTQYF

SEQ ID NO: 203 - protein, 11 aa, Artificial sequence
  TCR-CDR3 peptide sequence
CASSHQDTQYF

SEQ ID NO: 204 - protein, 10 aa, Artificial sequence
  TCR-CDR3 peptide sequence
CASSHYEQYF

SEQ ID NO: 205 - protein, 11 aa, Artificial sequence
  TCR-CDR3 peptide sequence
CASSVQDTQYF

SEQ ID NO: 206 - protein, 12 aa, Artificial sequence
  TCR-CDR3 peptide sequence
CASSLRGYEQYF

SEQ ID NO: 207 - protein, 12 aa, Artificial sequence
  TCR-CDR3 peptide sequence
CASSLEQYEQYF

SEQ ID NO: 208 - protein, 12 aa, Artificial sequence
  TCR-CDR3 peptide sequence
CASSLEGGEQYF

SEQ ID NO: 209 - protein, 12 aa, Artificial sequence
  TCR-CDR3 peptide sequence
CASSLGGDEQYF

SEQ ID NO: 210 - protein, 13 aa, Artificial sequence
  TCR-CDR3 peptide sequence
CASSWDSSYEQYF

SEQ ID NO: 211 - protein, 12 aa, Artificial sequence
  TCR-CDR3 peptide sequence
CASSLQGGEQYF

SEQ ID NO: 212 - protein, 12 aa, Artificial sequence
  TCR-CDR3 peptide sequence
CASSLGSYEQYF

SEQ ID NO: 213 - protein, 14 aa, Artificial sequence
  TCR-CDR3 peptide sequence
CASSLGDSSYEQYF

SEQ ID NO: 214 - protein, 11 aa, Artificial sequence
  TCR-CDR3 peptide sequence
CASSLGVEQYF

SEQ ID NO: 215 - protein, 13 aa, Artificial sequence
  TCR-CDR3 peptide sequence
CASSFGGQDTQYF

SEQ ID NO: 216 - protein, 13 aa, Artificial sequence
  TCR-CDR3 peptide sequence
CASSLGVQDTQYF

SEQ ID NO: 217 - protein, 14 aa, Artificial sequence
  TCR-CDR3 peptide sequence
CASSDWGSSYEQYF

SEQ ID NO: 218 - protein, 12 aa, Artificial sequence
  TCR-CDR3 peptide sequence
CASSLEQDTQYF

SEQ ID NO: 219 - protein, 12 aa, Artificial sequence
  TCR-CDR3 peptide sequence
CASSPDRDEQYF

SEQ ID NO: 220 - protein, 12 aa, Artificial sequence
  TCR-CDR3 peptide sequence
CASSLRGDTQYF

SEQ ID NO: 221 - protein, 13 aa, Artificial sequence
  TCR-CDR3 peptide sequence
CASSLAGGYEQYF

SEQ ID NO: 222 - protein, 13 aa, Artificial sequence
  TCR-CDR3 peptide sequence
CASSLGLGYEQYF

SEQ ID NO: 223 - protein, 11 aa, Artificial sequence
  TCR-CDR3 peptide sequence
CASSFDAEQFF

SEQ ID NO: 224 - protein, 10 aa, Artificial sequence
  TCR-CDR3 peptide sequence
CASSLREQYF

SEQ ID NO: 225 - protein, 12 aa, Artificial sequence
  TCR-CDR3 peptide sequence
CASSQQGYEQYF

SEQ ID NO: 226 - protein, 13 aa, Artificial sequence
  TCR-CDR3 peptide sequence
CASSQGNQDTQYF

SEQ ID NO: 227 - protein, 13 aa, Artificial sequence
  TCR-CDR3 peptide sequence
CASSRDRGDTQYF

SEQ ID NO: 228 - protein, 12 aa, Artificial sequence
  TCR-CDR3 peptide sequence
CASSLAQDTQYF

SEQ ID NO: 229 - protein, 13 aa, Artificial sequence
  TCR-CDR3 peptide sequence
CASSRDRQDTQYF

SEQ ID NO: 230 - protein, 11 aa, Artificial sequence
  TCR-CDR3 peptide sequence
CASSDEDTQYF

SEQ ID NO: 231 - protein, 11 aa, Artificial sequence
  TCR-CDR3 peptide sequence
CASGDNYEQYF

SEQ ID NO: 232 - protein, 14 aa, Artificial sequence
  TCR-CDR3 peptide sequence
CASSPTGGQDTQYF

SEQ ID NO: 233 - protein, 10 aa, Artificial sequence
  TCR-CDR3 peptide sequence
CASSPYEQYF

SEQ ID NO: 234 - protein, 11 aa, Artificial sequence
  TCR-CDR3 peptide sequence
CASSLNAEQFF

SEQ ID NO: 235 - protein, 11 aa, Artificial sequence
  TCR-CDR3 peptide sequence
CASSFGDTQYF

SEQ ID NO: 236 - protein, 13 aa, Artificial sequence
  TCR-CDR3 peptide sequence
CASSLQSSYEQYF

SEQ ID NO: 237 - protein, 12 aa, Artificial sequence
  TCR-CDR3 peptide sequence
CASSPGQDTQYF

SEQ ID NO: 238 - protein, 14 aa, Artificial sequence
  TCR-CDR3 peptide sequence
CASSPGQGNTEVFF

SEQ ID NO: 239 - protein, 13 aa, Artificial sequence
  TCR-CDR3 peptide sequence
CASSLDRGYEQYF

SEQ ID NO: 240 - protein, 11 aa, Artificial sequence
  TCR-CDR3 peptide sequence
CASSLNERLFF

SEQ ID NO: 241 - protein, 14 aa, Artificial sequence
  TCR-CDR3 peptide sequence
CASSLLGGQDTQYF

SEQ ID NO: 242 - protein, 11 aa, Artificial sequence
  TCR-CDR3 peptide sequence
CASSQEDTQYF

SEQ ID NO: 243 - protein, 14 aa, Artificial sequence
  TCR-CDR3 peptide sequence
CASSLGLGQDTQYF

SEQ ID NO: 244 - protein, 13 aa, Artificial sequence
  TCR-CDR3 peptide sequence
CASSLGLQDTQYF

SEQ ID NO: 245 - protein, 12 aa, Artificial sequence
  TCR-CDR3 peptide sequence
CASSLQGDEQYF

SEQ ID NO: 246 - protein, 14 aa, Artificial sequence
  TCR-CDR3 peptide sequence
CASSPGLGEDTQYF

SEQ ID NO: 247 - protein, 13 aa, Artificial sequence
  TCR-CDR3 peptide sequence
CASSLLGQDTQYF

SEQ ID NO: 248 - protein, 12 aa, Artificial sequence
  TCR-CDR3 peptide sequence
CASSLDGAEQFF

SEQ ID NO: 249 - protein, 13 aa, Artificial sequence
  TCR-CDR3 peptide sequence
CASSQDRDSDYTF

SEQ ID NO: 250 - protein, 13 aa, Artificial sequence
  TCR-CDR3 peptide sequence
CASSLTGEDTQYF

SEQ ID NO: 251 - protein, 11 aa, Artificial sequence
  TCR-CDR3 peptide sequence
CASSPGNTLYF

SEQ ID NO: 252 - protein, 11 aa, Artificial sequence
  TCR-CDR3 peptide sequence
CASGDRDEQYF

SEQ ID NO: 253 - protein, 10 aa, Artificial sequence
  TCR-CDR3 peptide sequence
CASGDGEQYF

SEQ ID NO: 254 - protein, 12 aa, Artificial sequence
  TCR-CDR3 peptide sequence
CASSLDKYEQYF

SEQ ID NO: 255 - protein, 13 aa, Artificial sequence
  TCR-CDR3 peptide sequence
CASSRDNSYEQYF

SEQ ID NO: 256 - protein, 14 aa, Artificial sequence
  TCR-CDR3 peptide sequence
CASSDAGGSYEQYF

SEQ ID NO: 257 - protein, 12 aa, Artificial sequence
  TCR-CDR3 peptide sequence
CASSGTGDEQYF

SEQ ID NO: 258 - protein, 13 aa, Artificial sequence
  TCR-CDR3 peptide sequence
CASSLVGAETLYF

SEQ ID NO: 259 - protein, 13 aa, Artificial sequence
  TCR-CDR3 peptide sequence
CASSLGGEDTQYF

SEQ ID NO: 260 - protein, 12 aa, Artificial sequence
  TCR-CDR3 peptide sequence
CASSLGDSDYTF

SEQ ID NO: 261 - protein, 12 aa, Artificial sequence
  TCR-CDR3 peptide sequence
CASSLVQDTQYF

SEQ ID NO: 262 - protein, 11 aa, Artificial sequence
  TCR-CDR3 peptide sequence
CASSTQDTQYF

SEQ ID NO: 263 - protein, 11 aa, Artificial sequence
  TCR-CDR3 peptide sequence
CASSHSYEQYF

SEQ ID NO: 264 - protein, 12 aa, Artificial sequence
  TCR-CDR3 peptide sequence
CASSSDRDEQYF

SEQ ID NO: 265 - protein, 13 aa, Artificial sequence
  TCR-CDR3 peptide sequence
CASSLEDSYEQYF

SEQ ID NO: 266 - protein, 12 aa, Artificial sequence
  TCR-CDR3 peptide sequence
CASSLQGDTQYF

SEQ ID NO: 267 - protein, 13 aa, Artificial sequence
  TCR-CDR3 peptide sequence
CASSLPGQDTQYF

SEQ ID NO: 268 - protein, 13 aa, Artificial sequence
  TCR-CDR3 peptide sequence
CASSLGQGYEQYF

SEQ ID NO: 269 - protein, 13 aa, Artificial sequence
  TCR-CDR3 peptide sequence
CASSPTGNSDYTF

SEQ ID NO: 270 - protein, 13 aa, Artificial sequence
  TCR-CDR3 peptide sequence
CASSRLGEDTQYF

SEQ ID NO: 271 - protein, 14 aa, Artificial sequence
  TCR-CDR3 peptide sequence
CASSRTGGAETLYF

SEQ ID NO: 272 - protein, 13 aa, Artificial sequence
  TCR-CDR3 peptide sequence
CASSLQGQDTQYF

SEQ ID NO: 273 - protein, 13 aa, Artificial sequence
  TCR-CDR3 peptide sequence
CASSPGQGYEQYF

SEQ ID NO: 274 - protein, 11 aa, Artificial sequence
  TCR-CDR3 peptide sequence
CASSPGDTQYF

SEQ ID NO: 275 - protein, 12 aa, Artificial sequence
  TCR-CDR3 peptide sequence
CASSRSSYEQYF

SEQ ID NO: 276 - protein, 12 aa, Artificial sequence
  TCR-CDR3 peptide sequence
CASSLSGDEQYF

SEQ ID NO: 277 - protein, 11 aa, Artificial sequence
  TCR-CDR3 peptide sequence
CASSSGYEQYF

SEQ ID NO: 278 - protein, 14 aa, Artificial sequence
  TCR-CDR3 peptide sequence
CASSQTGGQDTQYF

SEQ ID NO: 279 - protein, 12 aa, Artificial sequence
  TCR-CDR3 peptide sequence
CASSLTGYEQYF

SEQ ID NO: 280 - protein, 12 aa, Artificial sequence
  TCR-CDR3 peptide sequence
CASSLDIYEQYF

SEQ ID NO: 281 - protein, 12 aa, Artificial sequence
  TCR-CDR3 peptide sequence
CASSLSQDTQYF

SEQ ID NO: 282 - protein, 14 aa, Artificial sequence
  TCR-CDR3 peptide sequence
CASSLAGSSYEQYF

SEQ ID NO: 283 - protein, 12 aa, Artificial sequence
  TCR-CDR3 peptide sequence
CASSLGGREQYF

SEQ ID NO: 284 - protein, 12 aa, Artificial sequence
  TCR-CDR3 peptide sequence
CASSGTGDTQYF

SEQ ID NO: 285 - protein, 12 aa, Artificial sequence
  TCR-CDR3 peptide sequence
CASSGTEDTQYF

SEQ ID NO: 286 - protein, 11 aa, Artificial sequence
  TCR-CDR3 peptide sequence
CASSLTYEQYF

SEQ ID NO: 287 - protein, 11 aa, Artificial sequence
  TCR-CDR3 peptide sequence
CASSLDDTQYF

SEQ ID NO: 288 - protein, 13 aa, Artificial sequence
  TCR-CDR3 peptide sequence
CASSLGTEDTQYF

SEQ ID NO: 289 - protein, 9 aa, Artificial sequence
  TCR-CDR3 peptide sequence
CSAEDTQYF

SEQ ID NO: 290 - protein, 12 aa, Artificial sequence
  TCR-CDR3 peptide sequence
CASSRDIYEQYF

SEQ ID NO: 291 - protein, 11 aa, Artificial sequence
  TCR-CDR3 peptide sequence
CASSLRAEQFF

SEQ ID NO: 292 - protein, 13 aa, Artificial sequence
  TCR-CDR3 peptide sequence
CASSLLGEDTQYF

SEQ ID NO: 293 - protein, 11 aa, Artificial sequence
  TCR-CDR3 peptide sequence
CASSFGAEQFF

SEQ ID NO: 294 - protein, 11 aa, Artificial sequence
  TCR-CDR3 peptide sequence
CASSIQDTQYF

SEQ ID NO: 295 - protein, 11 aa, Artificial sequence
  TCR-CDR3 peptide sequence
CASSIQYEQYF

SEQ ID NO: 296 - protein, 12 aa, Artificial sequence
  TCR-CDR3 peptide sequence
CASSEGQYEQYF

SEQ ID NO: 297 - protein, 12 aa, Artificial sequence
  TCR-CDR3 peptide sequence
CASSDRGYEQYF

SEQ ID NO: 298 - protein, 10 aa, Artificial sequence
  TCR-CDR3 peptide sequence
CASGEGEQYF

SEQ ID NO: 299 - protein, 10 aa, Artificial sequence
  TCR-CDR3 peptide sequence
CASSDYEQYF

SEQ ID NO: 300 - protein, 11 aa, Artificial sequence
  TCR-CDR3 peptide sequence
CASSDAYEQYF

SEQ ID NO: 301 - protein, 10 aa, Artificial sequence
  TCR-CDR3 peptide sequence
CASGEYEQYF

SEQ ID NO: 302 - protein, 13 aa, Artificial sequence
  TCR-CDR3 peptide sequence
CASSLSGNTEVFF

SEQ ID NO: 303 - protein, 11 aa, Artificial sequence
  TCR-CDR3 peptide sequence
CASSLVGEQYF

SEQ ID NO: 304 - protein, 12 aa, Artificial sequence
  TCR-CDR3 peptide sequence
CASSIGQYEQYF

SEQ ID NO: 305 - protein, 11 aa, Artificial sequence
  TCR-CDR3 peptide sequence
CASSIGDTQYF

SEQ ID NO: 306 - protein, 12 aa, Artificial sequence
  TCR-CDR3 peptide sequence
CASSLDSAEQFF

SEQ ID NO: 307 - protein, 13 aa, Artificial sequence
  TCR-CDR3 peptide sequence
CASSQDRNTEVFF

SEQ ID NO: 308 - protein, 11 aa, Artificial sequence
  TCR-CDR3 peptide sequence
CASSEQDTQYF

SEQ ID NO: 309 - protein, 11 aa, Artificial sequence
  TCR-CDR3 peptide sequence
CASSGQDTQYF

SEQ ID NO: 310 - protein, 11 aa, Artificial sequence
  TCR-CDR3 peptide sequence
CASSLQGEQYF

SEQ ID NO: 311 - protein, 13 aa, Artificial sequence
  TCR-CDR3 peptide sequence
CASSLDNSYEQYF

SEQ ID NO: 312 - protein, 12 aa, Artificial sequence
  TCR-CDR3 peptide sequence
CASGDSSYEQYF

SEQ ID NO: 313 - protein, 12 aa, Artificial sequence
  TCR-CDR3 peptide sequence
CASGDASYEQYF

SEQ ID NO: 314 - protein, 14 aa, Artificial sequence
  TCR-CDR3 peptide sequence
CASSLTGGQDTQYF

SEQ ID NO: 315 - protein, 14 aa, Artificial sequence
  TCR-CDR3 peptide sequence
CASSLEGGQDTQYF

SEQ ID NO: 316 - protein, 13 aa, Artificial sequence
  TCR-CDR3 peptide sequence
CASSLTGGDTQYF

SEQ ID NO: 317 - protein, 13 aa, Artificial sequence
  TCR-CDR3 peptide sequence
CASSFTGEDTQYF

SEQ ID NO: 318 - protein, 11 aa, Artificial sequence
  TCR-CDR3 peptide sequence
CASSFRDTQYF

SEQ ID NO: 319 - protein, 11 aa, Artificial sequence
  TCR-CDR3 peptide sequence
CASSLEAEQFF

SEQ ID NO: 320 - protein, 12 aa, Artificial sequence
  TCR-CDR3 peptide sequence
CASSLGNYEQYF

SEQ ID NO: 321 - protein, 12 aa, Artificial sequence
  TCR-CDR3 peptide sequence
CASSRQGDTQYF

SEQ ID NO: 322 - protein, 13 aa, Artificial sequence
  TCR-CDR3 peptide sequence
CASSSTGGYEQYF

SEQ ID NO: 323 - protein, 14 aa, Artificial sequence
  TCR-CDR3 peptide sequence
CASSLGQSSYEQYF

SEQ ID NO: 324 - protein, 13 aa, Artificial sequence
  TCR-CDR3 peptide sequence
CASSLGDSYEQYF

SEQ ID NO: 325 - protein, 13 aa, Artificial sequence
  TCR-CDR3 peptide sequence
CASSWDSQDTQYF

SEQ ID NO: 326 - protein, 13 aa, Artificial sequence
  TCR-CDR3 peptide sequence
CASSPRGQDTQYF

SEQ ID NO: 327 - protein, 13 aa, Artificial sequence
  TCR-CDR3 peptide sequence
CASSLRSSYEQYF

SEQ ID NO: 328 - protein, 12 aa, Artificial sequence
  TCR-CDR3 peptide sequence
CASSRLGYEQYF

SEQ ID NO: 329 - protein, 14 aa, Artificial sequence
  TCR-CDR3 peptide sequence
CASSLPGGQDTQYF

SEQ ID NO: 330 - protein, 11 aa, Artificial sequence
  TCR-CDR3 peptide sequence
CASSLEYEQYF

SEQ ID NO: 331 - protein, 13 aa, Artificial sequence
  TCR-CDR3 peptide sequence
CASSSGSSYEQYF

SEQ ID NO: 332 - protein, 12 aa, Artificial sequence
  TCR-CDR3 peptide sequence
CASSRGQYEQYF

SEQ ID NO: 333 - protein, 10 aa, Artificial sequence
  TCR-CDR3 peptide sequence
CASSQGEQYF

SEQ ID NO: 334 - protein, 12 aa, Artificial sequence
  TCR-CDR3 peptide sequence
CASSLDGDTQYF

SEQ ID NO: 335 - protein, 13 aa, Artificial sequence
  TCR-CDR3 peptide sequence
CASRGQANTEVFF

SEQ ID NO: 336 - protein, 14 aa, Artificial sequence
  TCR-CDR3 peptide sequence
CASSPPGQQDTQYF

SEQ ID NO: 337 - protein, 13 aa, Artificial sequence
  TCR-CDR3 peptide sequence
CASSPGSSYEQYF

SEQ ID NO: 338 - protein, 12 aa, Artificial sequence
  TCR-CDR3 peptide sequence
CASSRDQDTQYF

SEQ ID NO: 339 - protein, 12 aa, Artificial sequence
  TCR-CDR3 peptide sequence
CASSRDFYEQYF

SEQ ID NO: 340 - protein, 11 aa, Artificial sequence
  TCR-CDR3 peptide sequence
CASSSEDTQYF

SEQ ID NO: 341 - protein, 12 aa, Artificial sequence
  TCR-CDR3 peptide sequence
CASSRDRYEQYF

SEQ ID NO: 342 - protein, 13 aa, Artificial sequence
  TCR-CDR3 peptide sequence
CASSEGSSYEQYF

SEQ ID NO: 343 - protein, 12 aa, Artificial sequence
  TCR-CDR3 peptide sequence
CASSLGDAEQFF

SEQ ID NO: 344 - protein, 12 aa, Artificial sequence
  TCR-CDR3 peptide sequence
CASSQDQDTQYF

SEQ ID NO: 345 - protein, 14 aa, Artificial sequence
  TCR-CDR3 peptide sequence
CASSPGTGQDTQYF

SEQ ID NO: 346 - protein, 14 aa, Artificial sequence
  TCR-CDR3 peptide sequence
CASSRTGDQDTQYF

SEQ ID NO: 347 - protein, 13 aa, Artificial sequence
  TCR-CDR3 peptide sequence
CASSLQGRDTQYF

SEQ ID NO: 348 - protein, 13 aa, Artificial sequence
  TCR-CDR3 peptide sequence
CASSWTGEDTQYF

SEQ ID NO: 349 - protein, 11 aa, Artificial sequence
  TCR-CDR3 peptide sequence
CASSWGYEQYF

SEQ ID NO: 350 - protein, 13 aa, Artificial sequence
  TCR-CDR3 peptide sequence
CASSLRGQDTQYF

SEQ ID NO: 351 - protein, 12 aa, Artificial sequence
  TCR-CDR3 peptide sequence
CASSLEGVEQYF

SEQ ID NO: 352 - protein, 11 aa, Artificial sequence
  TCR-CDR3 peptide sequence
CASSFKDTQYF

SEQ ID NO: 353 - protein, 12 aa, Artificial sequence
  TCR-CDR3 peptide sequence
CASSDEGYEQYF

SEQ ID NO: 354 - protein, 11 aa, Artificial sequence
  TCR-CDR3 peptide sequence
CASSDADTQYF

SEQ ID NO: 355 - protein, 12 aa, Artificial sequence
  TCR-CDR3 peptide sequence
CASSPDQDTQYF

SEQ ID NO: 356 - protein, 13 aa, Artificial sequence
  TCR-CDR3 peptide sequence
CASSPGGQDTQYF

SEQ ID NO: 357 - protein, 12 aa, Artificial sequence
  TCR-CDR3 peptide sequence
CASSLRQYEQYF

SEQ ID NO: 358 - protein, 12 aa, Artificial sequence
  TCR-CDR3 peptide sequence
CASSLVSYEQYF

SEQ ID NO: 359 - protein, 12 aa, Artificial sequence
  TCR-CDR3 peptide sequence
CASSSTGDEQYF

SEQ ID NO: 360 - protein, 10 aa, Artificial sequence
  TCR-CDR3 peptide sequence
CSADSYEQYF

SEQ ID NO: 361 - protein, 8 aa, Artificial sequence
  TCR-CDR3 peptide sequence
CASGEQYF

SEQ ID NO: 362 - protein, 13 aa, Artificial sequence
  TCR-CDR3 peptide sequence
CASSPDWGYEQYF

SEQ ID NO: 363 - protein, 13 aa, Artificial sequence
  TCR-CDR3 peptide sequence
CASSLQGEDTQYF

SEQ ID NO: 364 - protein, 12 aa, Artificial sequence
  TCR-CDR3 peptide sequence
CASSLAGGEQYF

SEQ ID NO: 365 - protein, 14 aa, Artificial sequence
  TCR-CDR3 peptide sequence
CASSLGTGQDTQYF

SEQ ID NO: 366 - protein, 12 aa, Artificial sequence
  TCR-CDR3 peptide sequence
CASSTGEDTQYF

SEQ ID NO: 367 - protein, 13 aa, Artificial sequence
  TCR-CDR3 peptide sequence
CASSPGTEDTQYF

SEQ ID NO: 368 - protein, 12 aa, Artificial sequence
  TCR-CDR3 peptide sequence
CASSDWGYEQYF

SEQ ID NO: 369 - protein, 12 aa, Artificial sequence
  TCR-CDR3 peptide sequence
CASSRDRDTQYF

SEQ ID NO: 370 - protein, 11 aa, Artificial sequence
  TCR-CDR3 peptide sequence
CASSQGYEQYF

SEQ ID NO: 371 - protein, 11 aa, Artificial sequence
  TCR-CDR3 peptide sequence
CASSDRYEQYF

SEQ ID NO: 372 - protein, 10 aa, Artificial sequence
  TCR-CDR3 peptide sequence
CASSYYEQYF

SEQ ID NO: 373 - protein, 12 aa, Artificial sequence
  TCR-CDR3 peptide sequence
CASSGQGYEQYF

SEQ ID NO: 374 - protein, 12 aa, Artificial sequence
  TCR-CDR3 peptide sequence
CASSQEGDTQYF

SEQ ID NO: 375 - protein, 13 aa, Artificial sequence
  TCR-CDR3 peptide sequence
CASSQDWEDTQYF

SEQ ID NO: 376 - protein, 14 aa, Artificial sequence
  TCR-CDR3 peptide sequence
CASSQDWGSYEQYF

SEQ ID NO: 377 - protein, 11 aa, Artificial sequence
  TCR-CDR3 peptide sequence
CASGDVDTQYF

SEQ ID NO: 378 - protein, 13 aa, Artificial sequence
  TCR-CDR3 peptide sequence
CASSLGQGDTQYF

SEQ ID NO: 379 - protein, 10 aa, Artificial sequence
  TCR-CDR3 peptide sequence
CASSDDEQYF

SEQ ID NO: 380 - protein, 14 aa, Artificial sequence
  TCR-CDR3 peptide sequence
CASSLTGGSYEQYF

SEQ ID NO: 381 - protein, 11 aa, Artificial sequence
  TCR-CDR3 peptide sequence
CASSLGSDYTF

SEQ ID NO: 382 - protein, 11 aa, Artificial sequence
  TCR-CDR3 peptide sequence
CASSQGAEQFF

SEQ ID NO: 383 - protein, 11 aa, Artificial sequence
  TCR-CDR3 peptide sequence
CASSSGDTQYF

SEQ ID NO: 384 - protein, 13 aa, Artificial sequence
  TCR-CDR3 peptide sequence
CASSLDRGAEQFF

SEQ ID NO: 385 - protein, 12 aa, Artificial sequence
  TCR-CDR3 peptide sequence
CASSSGGYEQYF

SEQ ID NO: 386 - protein, 12 aa, Artificial sequence
  TCR-CDR3 peptide sequence
CASSWDNYEQYF

SEQ ID NO: 387 - protein, 11 aa, Artificial sequence
  TCR-CDR3 peptide sequence
CASSFGDEQYF

SEQ ID NO: 388 - protein, 13 aa, Artificial sequence
  TCR-CDR3 peptide sequence
CASSRTGQDTQYF

SEQ ID NO: 389 - protein, 13 aa, Artificial sequence
  TCR-CDR3 peptide sequence
CASSLTGQDTQYF

SEQ ID NO: 390 - protein, 12 aa, Artificial sequence
  TCR-CDR3 peptide sequence
CASSPGGYEQYF

SEQ ID NO: 391 - protein, 13 aa, Artificial sequence
  TCR-CDR3 peptide sequence
CASSPGQGDTQYF

SEQ ID NO: 392 - protein, 13 aa, Artificial sequence
  TCR-CDR3 peptide sequence
CASSLPGGYEQYF

SEQ ID NO: 393 - protein, 11 aa, Artificial sequence
  TCR-CDR3 peptide sequence
CASSAQDTQYF

SEQ ID NO: 394 - protein, 13 aa, Artificial sequence
  TCR-CDR3 peptide sequence
CASSPTGGYEQYF

SEQ ID NO: 395 - protein, 14 aa, Artificial sequence
  TCR-CDR3 peptide sequence
CASSLTGSSYEQYF

SEQ ID NO: 396 - protein, 12 aa, Artificial sequence
  TCR-CDR3 peptide sequence
CASSLDRDTQYF

SEQ ID NO: 397 - protein, 12 aa, Artificial sequence
  TCR-CDR3 peptide sequence
CASSLGGGEQYF

SEQ ID NO: 398 - protein, 12 aa, Artificial sequence
  TCR-CDR3 peptide sequence
CASSSDRYEQYF

SEQ ID NO: 399 - protein, 11 aa, Artificial sequence
  TCR-CDR3 peptide sequence
CASSLDSEQYF

SEQ ID NO: 400 - protein, 12 aa, Artificial sequence
  TCR-CDR3 peptide sequence
CASSLAGDTQYF

SEQ ID NO: 401 - protein, 12 aa, Artificial sequence
  TCR-CDR3 peptide sequence
CASSLVGAEQFF

SEQ ID NO: 402 - protein, 11 aa, Artificial sequence
  TCR-CDR3 peptide sequence
CASSQDAEQFF

SEQ ID NO: 403 - protein, 13 aa, Artificial sequence
  TCR-CDR3 peptide sequence
CASSPTGQDTQYF

SEQ ID NO: 404 - protein, 14 aa, Artificial sequence
  TCR-CDR3 peptide sequence
CASSLSGGSYEQYF

SEQ ID NO: 405 - protein, 11 aa, Artificial sequence
  TCR-CDR3 peptide sequence
CASSDGYEQYF

SEQ ID NO: 406 - protein, 13 aa, Artificial sequence
  TCR-CDR3 peptide sequence
CASSPGLGYEQYF

SEQ ID NO: 407 - protein, 13 aa, Artificial sequence
  TCR-CDR3 peptide sequence
CASSRQGEDTQYF

SEQ ID NO: 408 - protein, 14 aa, Artificial sequence
  TCR-CDR3 peptide sequence
CASSLLGSSYEQYF

SEQ ID NO: 409 - protein, 13 aa, Artificial sequence
  TCR-CDR3 peptide sequence
CASSLGTQDTQYF

SEQ ID NO: 410 - protein, 14 aa, Artificial sequence
  TCR-CDR3 peptide sequence
CASSLSGGYAEQFF

SEQ ID NO: 411 - protein, 13 aa, Artificial sequence
  TCR-CDR3 peptide sequence
CASSLQGSYEQYF

SEQ ID NO: 412 - protein, 11 aa, Artificial sequence
  TCR-CDR3 peptide sequence
CASSLTDTQYF

SEQ ID NO: 413 - protein, 12 aa, Artificial sequence
  TCR-CDR3 peptide sequence
CASSFSSYEQYF

SEQ ID NO: 414 - protein, 12 aa, Artificial sequence
  TCR-CDR3 peptide sequence
CASSPDRGEQYF

SEQ ID NO: 415 - protein, 14 aa, Artificial sequence
  TCR-CDR3 peptide sequence
CASSLGGGQDTQYF

SEQ ID NO: 416 - protein, 11 aa, Artificial sequence
  TCR-CDR3 peptide sequence
CASGDIYEQYF

SEQ ID NO: 417 - protein, 12 aa, Artificial sequence
  TCR-CDR3 peptide sequence
CASSRDTYEQYF

SEQ ID NO: 418 - protein, 12 aa, Artificial sequence
  TCR-CDR3 peptide sequence
CASSSQGDTQYF

SEQ ID NO: 419 - protein, 10 aa, Artificial sequence
  TCR-CDR3 peptide sequence
CSADQDTQYF

SEQ ID NO: 420 - protein, 12 aa, Artificial sequence
  TCR-CDR3 peptide sequence
CASSPLGYEQYF

SEQ ID NO: 421 - protein, 13 aa, Artificial sequence
  TCR-CDR3 peptide sequence
CASSLRDNYEQYF

SEQ ID NO: 422 - protein, 11 aa, Artificial sequence
  TCR-CDR3 peptide sequence
CASSLAYEQYF

SEQ ID NO: 423 - protein, 13 aa, Artificial sequence
  TCR-CDR3 peptide sequence
CASSLGQSYEQYF

SEQ ID NO: 424 - protein, 11 aa, Artificial sequence
  TCR-CDR3 peptide sequence
CASSLVDTQYF

SEQ ID NO: 425 - protein, 11 aa, Artificial sequence
  TCR-CDR3 peptide sequence
CASGEGDTQYF

SEQ ID NO: 426 - protein, 13 aa, Artificial sequence
  TCR-CDR3 peptide sequence
CASSRTGVYEQYF

SEQ ID NO: 427 - protein, 12 aa, Artificial sequence
  TCR-CDR3 peptide sequence
CASSQDRDEQYF

SEQ ID NO: 428 - protein, 11 aa, Artificial sequence
  TCR-CDR3 peptide sequence
CASSEGYEQYF

SEQ ID NO: 429 - protein, 12 aa, Artificial sequence
  TCR-CDR3 peptide sequence
CASGESSYEQYF

SEQ ID NO: 430 - protein, 13 aa, Artificial sequence
  TCR-CDR3 peptide sequence
CASSTGNQDTQYF

SEQ ID NO: 431 - protein, 12 aa, Artificial sequence
  TCR-CDR3 peptide sequence
CASSLNSYEQYF

SEQ ID NO: 432 - protein, 10 aa, Artificial sequence
  TCR-CDR3 peptide sequence
CASSYAEQFF

SEQ ID NO: 433 - protein, 13 aa, Artificial sequence
  TCR-CDR3 peptide sequence
CASSLGTGDTQYF

SEQ ID NO: 434 - protein, 10 aa, Artificial sequence
  TCR-CDR3 peptide sequence
CSAGQYEQYF

SEQ ID NO: 435 - protein, 12 aa, Artificial sequence
  TCR-CDR3 peptide sequence
CASSQTGYEQYF

SEQ ID NO: 436 - protein, 14 aa, Artificial sequence
  TCR-CDR3 peptide sequence
CASSLGLGEDTQYF

SEQ ID NO: 437 - protein, 12 aa, Artificial sequence
  TCR-CDR3 peptide sequence
CASSQDRYEQYF

SEQ ID NO: 438 - protein, 11 aa, Artificial sequence
  TCR-CDR3 peptide sequence
CASSFGETLYF

SEQ ID NO: 439 - protein, 13 aa, Artificial sequence
  TCR-CDR3 peptide sequence
CASSLGTGYEQYF

SEQ ID NO: 440 - protein, 12 aa, Artificial sequence
  TCR-CDR3 peptide sequence
CASGAFNQAPLF

SEQ ID NO: 441 - protein, 10 aa, Artificial sequence
  TCR-CDR3 peptide sequence
CASGDAEQFF

SEQ ID NO: 442 - protein, 14 aa, Artificial sequence
  TCR-CDR3 peptide sequence
CASGDAGGQDTQYF

SEQ ID NO: 443 - protein, 12 aa, Artificial sequence
  TCR-CDR3 peptide sequence
CASGDAGNTLYF

SEQ ID NO: 444 - protein, 13 aa, Artificial sequence
  TCR-CDR3 peptide sequence
CASGDAGQNTLYF

SEQ ID NO: 445 - protein, 14 aa, Artificial sequence
  TCR-CDR3 peptide sequence
CASGDAGSQNTLYF

SEQ ID NO: 446 - protein, 12 aa, Artificial sequence
  TCR-CDR3 peptide sequence
CASGDAGYEQYF

SEQ ID NO: 447 - protein, 13 aa, Artificial sequence
  TCR-CDR3 peptide sequence
CASGDGSQNTLYF

SEQ ID NO: 448 - protein, 12 aa, Artificial sequence
  TCR-CDR3 peptide sequence
CASGDNYAEQFF

SEQ ID NO: 449 - protein, 11 aa, Artificial sequence
  TCR-CDR3 peptide sequence
CASGDQDTQYF

SEQ ID NO: 450 - protein, 11 aa, Artificial sequence
  TCR-CDR3 peptide sequence
CASGDRDTQYF

SEQ ID NO: 451 - protein, 12 aa, Artificial sequence
  TCR-CDR3 peptide sequence
CASGDSAETLYF

SEQ ID NO: 452 - protein, 12 aa, Artificial sequence
  TCR-CDR3 peptide sequence
CASGDSGNTLYF

SEQ ID NO: 453 - protein, 13 aa, Artificial sequence
  TCR-CDR3 peptide sequence
CASGDSSQNTLYF

SEQ ID NO: 454 - protein, 14 aa, Artificial sequence
  TCR-CDR3 peptide sequence
CASGDWGSAETLYF

SEQ ID NO: 455 - protein, 14 aa, Artificial sequence
  TCR-CDR3 peptide sequence
CASGDWGSQNTLYF

SEQ ID NO: 456 - protein, 12 aa, Artificial sequence
  TCR-CDR3 peptide sequence
CASRDSAETLYF

SEQ ID NO: 457 - protein, 15 aa, Artificial sequence
  TCR-CDR3 peptide sequence
CASRPGTANTGQLYF

SEQ ID NO: 458 - protein, 10 aa, Artificial sequence
  TCR-CDR3 peptide sequence
CASSAETLYF

SEQ ID NO: 459 - protein, 13 aa, Artificial sequence
  TCR-CDR3 peptide sequence
CASSDRGQNTLYF

SEQ ID NO: 460 - protein, 12 aa, Artificial sequence
  TCR-CDR3 peptide sequence
CASSDSAETLYF

SEQ ID NO: 461 - protein, 12 aa, Artificial sequence
  TCR-CDR3 peptide sequence
CASSDSQNTLYF

SEQ ID NO: 462 - protein, 13 aa, Artificial sequence
  TCR-CDR3 peptide sequence
CASSDSSAETLYF

SEQ ID NO: 463 - protein, 13 aa, Artificial sequence
  TCR-CDR3 peptide sequence
CASSDSSQNTLYF

SEQ ID NO: 464 - protein, 13 aa, Artificial sequence
  TCR-CDR3 peptide sequence
CASSFDSQNTLYF

SEQ ID NO: 465 - protein, 12 aa, Artificial sequence
  TCR-CDR3 peptide sequence
CASSFGQNTLYF

SEQ ID NO: 466 - protein, 13 aa, Artificial sequence
  TCR-CDR3 peptide sequence
CASSFGSQNTLYF

SEQ ID NO: 467 - protein, 13 aa, Artificial sequence
  TCR-CDR3 peptide sequence
CASSFQANTEVFF

SEQ ID NO: 468 - protein, 12 aa, Artificial sequence
  TCR-CDR3 peptide sequence
CASSFSAETLYF

SEQ ID NO: 469 - protein, 12 aa, Artificial sequence
  TCR-CDR3 peptide sequence
CASSFSQNTLYF

SEQ ID NO: 470 - protein, 13 aa, Artificial sequence
  TCR-CDR3 peptide sequence
CASSGTANSDYTF

SEQ ID NO: 471 - protein, 13 aa, Artificial sequence
  TCR-CDR3 peptide sequence
CASSGTTNSDYTF

SEQ ID NO: 472 - protein, 12 aa, Artificial sequence
  TCR-CDR3 peptide sequence
CASSHSAETLYF

SEQ ID NO: 473 - protein, 12 aa, Artificial sequence
  TCR-CDR3 peptide sequence
CASSHSQNTLYF

SEQ ID NO: 474 - protein, 13 aa, Artificial sequence
  TCR-CDR3 peptide sequence
CASSLAANSDYTF

SEQ ID NO: 475 - protein, 14 aa, Artificial sequence
  TCR-CDR3 peptide sequence
CASSLAGNYAEQFF

SEQ ID NO: 476 - protein, 14 aa, Artificial sequence
  TCR-CDR3 peptide sequence
CASSLAGSQNTLYF

SEQ ID NO: 477 - protein, 12 aa, Artificial sequence
  TCR-CDR3 peptide sequence
CASSLANSDYTF

SEQ ID NO: 478 - protein, 13 aa, Artificial sequence
  TCR-CDR3 peptide sequence
CASSLANTGQLYF

SEQ ID NO: 479 - protein, 13 aa, Artificial sequence
  TCR-CDR3 peptide sequence
CASSLASAETLYF

SEQ ID NO: 480 - protein, 11 aa, Artificial sequence
  TCR-CDR3 peptide sequence
CASSLDERLFF

SEQ ID NO: 481 - protein, 14 aa, Artificial sequence
  TCR-CDR3 peptide sequence
CASSLDGSQNTLYF

SEQ ID NO: 482 - protein, 13 aa, Artificial sequence
  TCR-CDR3 peptide sequence
CASSLDNQDTQYF

SEQ ID NO: 483 - protein, 14 aa, Artificial sequence
  TCR-CDR3 peptide sequence
CASSLDNSGNTLYF

SEQ ID NO: 484 - protein, 13 aa, Artificial sequence
  TCR-CDR3 peptide sequence
CASSLDNYAEQFF

SEQ ID NO: 485 - protein, 13 aa, Artificial sequence
  TCR-CDR3 peptide sequence
CASSLDRYAEQFF

SEQ ID NO: 486 - protein, 13 aa, Artificial sequence
  TCR-CDR3 peptide sequence
CASSLDSAETLYF

SEQ ID NO: 487 - protein, 11 aa, Artificial sequence
  TCR-CDR3 peptide sequence
CASSLDSDYTF

SEQ ID NO: 488 - protein, 13 aa, Artificial sequence
  TCR-CDR3 peptide sequence
CASSLDSQNTLYF

SEQ ID NO: 489 - protein, 14 aa, Artificial sequence
  TCR-CDR3 peptide sequence
CASSLDSSAETLYF

SEQ ID NO: 490 - protein, 14 aa, Artificial sequence
  TCR-CDR3 peptide sequence
CASSLDSSGNTLYF

SEQ ID NO: 491 - protein, 14 aa, Artificial sequence
  TCR-CDR3 peptide sequence
CASSLDSSQNTLYF

SEQ ID NO: 492 - protein, 13 aa, Artificial sequence
  TCR-CDR3 peptide sequence
CASSLDSYAEQFF

SEQ ID NO: 493 - protein, 11 aa, Artificial sequence
  TCR-CDR3 peptide sequence
CASSLDTEVFF

SEQ ID NO: 494 - protein, 12 aa, Artificial sequence
  TCR-CDR3 peptide sequence
CASSLDTGQLYF

SEQ ID NO: 495 - protein, 15 aa, Artificial sequence
  TCR-CDR3 peptide sequence
CASSLDWGNYAEQFF

SEQ ID NO: 496 - protein, 14 aa, Artificial sequence
  TCR-CDR3 peptide sequence
CASSLDWGQDTQYF

SEQ ID NO: 497 - protein, 15 aa, Artificial sequence
  TCR-CDR3 peptide sequence
CASSLDWGSAETLYF

SEQ ID NO: 498 - protein, 13 aa, Artificial sequence
  TCR-CDR3 peptide sequence
CASSLEANSDYTF

SEQ ID NO: 499 - protein, 14 aa, Artificial sequence
  TCR-CDR3 peptide sequence
CASSLEDSGNTLYF

SEQ ID NO: 500 - protein, 13 aa, Artificial sequence
  TCR-CDR3 peptide sequence
CASSLEGAETLYF

SEQ ID NO: 501 - protein, 13 aa, Artificial sequence
  TCR-CDR3 peptide sequence
CASSLEGNSDYTF

SEQ ID NO: 502 - protein, 13 aa, Artificial sequence
  TCR-CDR3 peptide sequence
CASSLEGNTEVFF

SEQ ID NO: 503 - protein, 14 aa, Artificial sequence
  TCR-CDR3 peptide sequence
CASSLEGNTGQLYF

SEQ ID NO: 504 - protein, 12 aa, Artificial sequence
  TCR-CDR3 peptide sequence
CASSLEGNTLYF

SEQ ID NO: 505 - protein, 14 aa, Artificial sequence
  TCR-CDR3 peptide sequence
CASSLEGNYAEQFF

SEQ ID NO: 506 - protein, 14 aa, Artificial sequence
  TCR-CDR3 peptide sequence
CASSLEGSGNTLYF

SEQ ID NO: 507 - protein, 14 aa, Artificial sequence
  TCR-CDR3 peptide sequence
CASSLEGSQNTLYF

SEQ ID NO: 508 - protein, 13 aa, Artificial sequence
  TCR-CDR3 peptide sequence
CASSLEGYAEQFF

SEQ ID NO: 509 - protein, 14 aa, Artificial sequence
  TCR-CDR3 peptide sequence
CASSLESANSDYTF

SEQ ID NO: 510 - protein, 12 aa, Artificial sequence
  TCR-CDR3 peptide sequence
CASSLGAETLYF

SEQ ID NO: 511 - protein, 13 aa, Artificial sequence
  TCR-CDR3 peptide sequence
CASSLGANSDYTF

SEQ ID NO: 512 - protein, 13 aa, Artificial sequence
  TCR-CDR3 peptide sequence
CASSLGDQDTQYF

SEQ ID NO: 513 - protein, 12 aa, Artificial sequence
  TCR-CDR3 peptide sequence
CASSLGENTLYF

SEQ ID NO: 514 - protein, 11 aa, Artificial sequence
  TCR-CDR3 peptide sequence
CASSLGERLFF

SEQ ID NO: 515 - protein, 11 aa, Artificial sequence
  TCR-CDR3 peptide sequence
CASSLGETLYF

SEQ ID NO: 516 - protein, 13 aa, Artificial sequence
  TCR-CDR3 peptide sequence
CASSLGGAETLYF

SEQ ID NO: 517 - protein, 13 aa, Artificial sequence
  TCR-CDR3 peptide sequence
CASSLGGNSDYTF

SEQ ID NO: 518 - protein, 13 aa, Artificial sequence
  TCR-CDR3 peptide sequence
CASSLGGNTEVFF

SEQ ID NO: 519 - protein, 12 aa, Artificial sequence
  TCR-CDR3 peptide sequence
CASSLGGNTLYF

SEQ ID NO: 520 - protein, 14 aa, Artificial sequence
  TCR-CDR3 peptide sequence
CASSLGGSAETLYF

SEQ ID NO: 521 - protein, 14 aa, Artificial sequence
  TCR-CDR3 peptide sequence
CASSLGGSQNTLYF

SEQ ID NO: 522 - protein, 13 aa, Artificial sequence
  TCR-CDR3 peptide sequence
CASSLGNQDTQYF

SEQ ID NO: 523 - protein, 14 aa, Artificial sequence
  TCR-CDR3 peptide sequence
CASSLGNSGNTLYF

SEQ ID NO: 524 - protein, 12 aa, Artificial sequence
  TCR-CDR3 peptide sequence
CASSLGNTEVFF

SEQ ID NO: 525 - protein, 13 aa, Artificial sequence
  TCR-CDR3 peptide sequence
CASSLGNTGQLYF

SEQ ID NO: 526 - protein, 11 aa, Artificial sequence
  TCR-CDR3 peptide sequence
CASSLGNTLYF

SEQ ID NO: 527 - protein, 13 aa, Artificial sequence
  TCR-CDR3 peptide sequence
CASSLGQNSDYTF

SEQ ID NO: 528 - protein, 12 aa, Artificial sequence
  TCR-CDR3 peptide sequence
CASSLGQNTLYF

SEQ ID NO: 529 - protein, 14 aa, Artificial sequence
  TCR-CDR3 peptide sequence
CASSLGQNYAEQFF

SEQ ID NO: 530 - protein, 13 aa, Artificial sequence
  TCR-CDR3 peptide sequence
CASSLGQQNTLYF

SEQ ID NO: 531 - protein, 14 aa, Artificial sequence
  TCR-CDR3 peptide sequence
CASSLGQSQNTLYF

SEQ ID NO: 532 - protein, 12 aa, Artificial sequence
  TCR-CDR3 peptide sequence
CASSLGQTEVFF

SEQ ID NO: 533 - protein, 13 aa, Artificial sequence
  TCR-CDR3 peptide sequence
CASSLGSAETLYF

SEQ ID NO: 534 - protein, 13 aa, Artificial sequence
  TCR-CDR3 peptide sequence
CASSLGSQNTLYF

SEQ ID NO: 535 - protein, 14 aa, Artificial sequence
  TCR-CDR3 peptide sequence
CASSLGSSAETLYF

SEQ ID NO: 536 - protein, 14 aa, Artificial sequence
  TCR-CDR3 peptide sequence
CASSLGTANSDYTF

SEQ ID NO: 537 - protein, 14 aa, Artificial sequence
  TCR-CDR3 peptide sequence
CASSLGTTNSDYTF

SEQ ID NO: 538 - protein, 14 aa, Artificial sequence
  TCR-CDR3 peptide sequence
CASSLLGNYAEQFF

SEQ ID NO: 539 - protein, 12 aa, Artificial sequence
  TCR-CDR3 peptide sequence
CASSLNQDTQYF

SEQ ID NO: 540 - protein, 13 aa, Artificial sequence
  TCR-CDR3 peptide sequence
CASSLNSAETLYF

SEQ ID NO: 541 - protein, 12 aa, Artificial sequence
  TCR-CDR3 peptide sequence
CASSLNTGQLYF

SEQ ID NO: 542 - protein, 13 aa, Artificial sequence
  TCR-CDR3 peptide sequence
CASSLQANSDYTF

SEQ ID NO: 543 - protein, 13 aa, Artificial sequence
  TCR-CDR3 peptide sequence
CASSLQANTEVFF

SEQ ID NO: 544 - protein, 13 aa, Artificial sequence
  TCR-CDR3 peptide sequence
CASSLQGAETLYF

SEQ ID NO: 545 - protein, 14 aa, Artificial sequence
  TCR-CDR3 peptide sequence
CASSLQGAGNTLYF

SEQ ID NO: 546 - protein, 14 aa, Artificial sequence
  TCR-CDR3 peptide sequence
CASSLQGANTEVFF

SEQ ID NO: 547 - protein, 13 aa, Artificial sequence
  TCR-CDR3 peptide sequence
CASSLQGNSDYTF

SEQ ID NO: 548 - protein, 13 aa, Artificial sequence
  TCR-CDR3 peptide sequence
CASSLQGNTEVFF

SEQ ID NO: 549 - protein, 14 aa, Artificial sequence
  TCR-CDR3 peptide sequence
CASSLQGNTGQLYF

SEQ ID NO: 550 - protein, 14 aa, Artificial sequence
  TCR-CDR3 peptide sequence
CASSLQGSAETLYF

SEQ ID NO: 551 - protein, 14 aa, Artificial sequence
  TCR-CDR3 peptide sequence
CASSLQGSGNTLYF

SEQ ID NO: 552 - protein, 14 aa, Artificial sequence
  TCR-CDR3 peptide sequence
CASSLQGSQNTLYF

SEQ ID NO: 553 - protein, 12 aa, Artificial sequence
  TCR-CDR3 peptide sequence
CASSLQGTEVFF

SEQ ID NO: 554 - protein, 11 aa, Artificial sequence
  TCR-CDR3 peptide sequence
CASSLQNTLYF

SEQ ID NO: 555 - protein, 14 aa, Artificial sequence
  TCR-CDR3 peptide sequence
CASSLRGSQNTLYF

SEQ ID NO: 556 - protein, 12 aa, Artificial sequence
  TCR-CDR3 peptide sequence
CASSLSAETLYF

SEQ ID NO: 557 - protein, 12 aa, Artificial sequence
  TCR-CDR3 peptide sequence
CASSLSGNTLYF

SEQ ID NO: 558 - protein, 15 aa, Artificial sequence
  TCR-CDR3 peptide sequence
CASSLSGSNYAEQFF

SEQ ID NO: 559 - protein, 12 aa, Artificial sequence
  TCR-CDR3 peptide sequence
CASSLSQNTLYF

SEQ ID NO: 560 - protein, 13 aa, Artificial sequence
  TCR-CDR3 peptide sequence
CASSLSQQNTLYF

SEQ ID NO: 561 - protein, 13 aa, Artificial sequence
  TCR-CDR3 peptide sequence
CASSLSSQNTLYF

SEQ ID NO: 562 - protein, 13 aa, Artificial sequence
  TCR-CDR3 peptide sequence
CASSLTANSDYTF

SEQ ID NO: 563 - protein, 13 aa, Artificial sequence
  TCR-CDR3 peptide sequence
CASSLTANTEVFF

SEQ ID NO: 564 - protein, 14 aa, Artificial sequence
  TCR-CDR3 peptide sequence
CASSLTDYNSPLYF

SEQ ID NO: 565 - protein, 14 aa, Artificial sequence
  TCR-CDR3 peptide sequence
CASSLTGGYAEQFF

SEQ ID NO: 566 - protein, 14 aa, Artificial sequence
  TCR-CDR3 peptide sequence
CASSLTGNYAEQFF

SEQ ID NO: 567 - protein, 14 aa, Artificial sequence
  TCR-CDR3 peptide sequence
CASSLTGSQNTLYF

SEQ ID NO: 568 - protein, 13 aa, Artificial sequence
  TCR-CDR3 peptide sequence
CASSLTSAETLYF

SEQ ID NO: 569 - protein, 14 aa, Artificial sequence
  TCR-CDR3 peptide sequence
CASSLWGSQNTLYF

SEQ ID NO: 570 - protein, 13 aa, Artificial sequence
  TCR-CDR3 peptide sequence
CASSPDSAETLYF

SEQ ID NO: 571 - protein, 14 aa, Artificial sequence
  TCR-CDR3 peptide sequence
CASSPDSSGNTLYF

SEQ ID NO: 572 - protein, 14 aa, Artificial sequence
  TCR-CDR3 peptide sequence
CASSPDSSQNTLYF

SEQ ID NO: 573 - protein, 14 aa, Artificial sequence
  TCR-CDR3 peptide sequence
CASSPDWGENTLYF

SEQ ID NO: 574 - protein, 15 aa, Artificial sequence
  TCR-CDR3 peptide sequence
CASSPDWGNYAEQFF

SEQ ID NO: 575 - protein, 13 aa, Artificial sequence
  TCR-CDR3 peptide sequence
CASSPGDQDTQYF

SEQ ID NO: 576 - protein, 13 aa, Artificial sequence
  TCR-CDR3 peptide sequence
CASSPGGQNTLYF

SEQ ID NO: 577 - protein, 12 aa, Artificial sequence
  TCR-CDR3 peptide sequence
CASSPGHERLFF

SEQ ID NO: 578 - protein, 14 aa, Artificial sequence
  TCR-CDR3 peptide sequence
CASSPGQGNSDYTF

SEQ ID NO: 579 - protein, 14 aa, Artificial sequence
  TCR-CDR3 peptide sequence
CASSPGQGYAEQFF

SEQ ID NO: 580 - protein, 13 aa, Artificial sequence
  TCR-CDR3 peptide sequence
CASSPGQNTEVFF

SEQ ID NO: 581 - protein, 14 aa, Artificial sequence
  TCR-CDR3 peptide sequence
CASSPGQNYAEQFF

SEQ ID NO: 582 - protein, 14 aa, Artificial sequence
  TCR-CDR3 peptide sequence
CASSPGQSQNTLYF

SEQ ID NO: 583 - protein, 13 aa, Artificial sequence
  TCR-CDR3 peptide sequence
CASSPGSQNTLYF

SEQ ID NO: 584 - protein, 14 aa, Artificial sequence
  TCR-CDR3 peptide sequence
CASSPGTANTEVFF

SEQ ID NO: 585 - protein, 12 aa, Artificial sequence
  TCR-CDR3 peptide sequence
CASSPNTGQLYF

SEQ ID NO: 586 - protein, 12 aa, Artificial sequence
  TCR-CDR3 peptide sequence
CASSPNYAEQFF

SEQ ID NO: 587 - protein, 13 aa, Artificial sequence
  TCR-CDR3 peptide sequence
CASSPQGNTEVFF

SEQ ID NO: 588 - protein, 12 aa, Artificial sequence
  TCR-CDR3 peptide sequence
CASSPSAETLYF

SEQ ID NO: 589 - protein, 12 aa, Artificial sequence
  TCR-CDR3 peptide sequence
CASSPSQNTLYF

SEQ ID NO: 590 - protein, 13 aa, Artificial sequence
  TCR-CDR3 peptide sequence
CASSPSSAETLYF

SEQ ID NO: 591 - protein, 13 aa, Artificial sequence
  TCR-CDR3 peptide sequence
CASSPTANSDYTF

SEQ ID NO: 592 - protein, 14 aa, Artificial sequence
  TCR-CDR3 peptide sequence
CASSPTASQNTLYF

SEQ ID NO: 593 - protein, 13 aa, Artificial sequence
  TCR-CDR3 peptide sequence
CASSPTGAETLYF

SEQ ID NO: 594 - protein, 14 aa, Artificial sequence
  TCR-CDR3 peptide sequence
CASSPTGSQNTLYF

SEQ ID NO: 595 - protein, 11 aa, Artificial sequence
  TCR-CDR3 peptide sequence
CASSPYAEQFF

SEQ ID NO: 596 - protein, 11 aa, Artificial sequence
  TCR-CDR3 peptide sequence
CASSQDTEVFF

SEQ ID NO: 597 - protein, 14 aa, Artificial sequence
  TCR-CDR3 peptide sequence
CASSQDWGQDTQYF

SEQ ID NO: 598 - protein, 14 aa, Artificial sequence
  TCR-CDR3 peptide sequence
CASSQEASNSDYTF

SEQ ID NO: 599 - protein, 13 aa, Artificial sequence
  TCR-CDR3 peptide sequence
CASSQGLGDTLYF

SEQ ID NO: 600 - protein, 13 aa, Artificial sequence
  TCR-CDR3 peptide sequence
CASSQGQNTEVFF

SEQ ID NO: 601 - protein, 13 aa, Artificial sequence
  TCR-CDR3 peptide sequence
CASSQGSQNTLYF

SEQ ID NO: 602 - protein, 12 aa, Artificial sequence
  TCR-CDR3 peptide sequence
CASSQNTGQLYF

SEQ ID NO: 603 - protein, 14 aa, Artificial sequence
  TCR-CDR3 peptide sequence
CASSQQGSQNTLYF

SEQ ID NO: 604 - protein, 15 aa, Artificial sequence
  TCR-CDR3 peptide sequence
CASSQSLDNQDTQYF

SEQ ID NO: 605 - protein, 13 aa, Artificial sequence
  TCR-CDR3 peptide sequence
CASSRDIQDTQYF

SEQ ID NO: 606 - protein, 13 aa, Artificial sequence
  TCR-CDR3 peptide sequence
CASSRDIYAEQFF

SEQ ID NO: 607 - protein, 13 aa, Artificial sequence
  TCR-CDR3 peptide sequence
CASSRDNNERLFF

SEQ ID NO: 608 - protein, 13 aa, Artificial sequence
  TCR-CDR3 peptide sequence
CASSRDNQDTQYF

SEQ ID NO: 609 - protein, 13 aa, Artificial sequence
  TCR-CDR3 peptide sequence
CASSRDNYAEQFF

SEQ ID NO: 610 - protein, 13 aa, Artificial sequence
  TCR-CDR3 peptide sequence
CASSRDRDTEVFF

SEQ ID NO: 611 - protein, 13 aa, Artificial sequence
  TCR-CDR3 peptide sequence
CASSRDSAETLYF

SEQ ID NO: 612 - protein, 13 aa, Artificial sequence
  TCR-CDR3 peptide sequence
CASSRDSQNTLYF

SEQ ID NO: 613 - protein, 14 aa, Artificial sequence
  TCR-CDR3 peptide sequence
CASSRDSSAETLYF

SEQ ID NO: 614 - protein, 13 aa, Artificial sequence
  TCR-CDR3 peptide sequence
CASSRDSYAEQFF

SEQ ID NO: 615 - protein, 14 aa, Artificial sequence
  TCR-CDR3 peptide sequence
CASSRDWEQNTLYF

SEQ ID NO: 616 - protein, 15 aa, Artificial sequence
  TCR-CDR3 peptide sequence
CASSRDWGNYAEQFF

SEQ ID NO: 617 - protein, 14 aa, Artificial sequence
  TCR-CDR3 peptide sequence
CASSRDWGQDTQYF

SEQ ID NO: 618 - protein, 12 aa, Artificial sequence
  TCR-CDR3 peptide sequence
CASSRGQNTLYF

SEQ ID NO: 619 - protein, 13 aa, Artificial sequence
  TCR-CDR3 peptide sequence
CASSRGSAETLYF

SEQ ID NO: 620 - protein, 13 aa, Artificial sequence
  TCR-CDR3 peptide sequence
CASSRQANTEVFF

SEQ ID NO: 621 - protein, 14 aa, Artificial sequence
  TCR-CDR3 peptide sequence
CASSRQGANSDYTF

SEQ ID NO: 622 - protein, 13 aa, Artificial sequence
  TCR-CDR3 peptide sequence
CASSRQGNSDYTF

SEQ ID NO: 623 - protein, 13 aa, Artificial sequence
  TCR-CDR3 peptide sequence
CASSRQGNTEVFF

SEQ ID NO: 624 - protein, 14 aa, Artificial sequence
  TCR-CDR3 peptide sequence
CASSRQGNTGQLYF

SEQ ID NO: 625 - protein, 14 aa, Artificial sequence
  TCR-CDR3 peptide sequence
CASSRQGNYAEQFF

SEQ ID NO: 626 - protein, 14 aa, Artificial sequence
  TCR-CDR3 peptide sequence
CASSRQGSQNTLYF

SEQ ID NO: 627 - protein, 12 aa, Artificial sequence
  TCR-CDR3 peptide sequence
CASSRQNSDYTF

SEQ ID NO: 628 - protein, 12 aa, Artificial sequence
  TCR-CDR3 peptide sequence
CASSRQNTEVFF

SEQ ID NO: 629 - protein, 12 aa, Artificial sequence
  TCR-CDR3 peptide sequence
CASSRSQNTLYF

SEQ ID NO: 630 - protein, 12 aa, Artificial sequence
  TCR-CDR3 peptide sequence
CASSRVGSDYTF

SEQ ID NO: 631 - protein, 11 aa, Artificial sequence
  TCR-CDR3 peptide sequence
CASSSAETLYF

SEQ ID NO: 632 - protein, 13 aa, Artificial sequence
  TCR-CDR3 peptide sequence
CASSSDSAETLYF

SEQ ID NO: 633 - protein, 15 aa, Artificial sequence
  TCR-CDR3 peptide sequence
CASSSDWGNYAEQFF

SEQ ID NO: 634 - protein, 13 aa, Artificial sequence
  TCR-CDR3 peptide sequence
CASSSGDQDTQYF

SEQ ID NO: 635 - protein, 13 aa, Artificial sequence
  TCR-CDR3 peptide sequence
CASSSGQNTEVFF

SEQ ID NO: 636 - protein, 13 aa, Artificial sequence
  TCR-CDR3 peptide sequence
CASSSGSQNTLYF

SEQ ID NO: 637 - protein, 14 aa, Artificial sequence
  TCR-CDR3 peptide sequence
CASSSGTANTEVFF

SEQ ID NO: 638 - protein, 12 aa, Artificial sequence
  TCR-CDR3 peptide sequence
CASSSNTGQLYF

SEQ ID NO: 639 - protein, 13 aa, Artificial sequence
  TCR-CDR3 peptide sequence
CASSSQGAETLYF

SEQ ID NO: 640 - protein, 11 aa, Artificial sequence
  TCR-CDR3 peptide sequence
CASSSQNTLYF

SEQ ID NO: 641 - protein, 12 aa, Artificial sequence
  TCR-CDR3 peptide sequence
CASSSSAETLYF

SEQ ID NO: 642 - protein, 12 aa, Artificial sequence
  TCR-CDR3 peptide sequence
CASSSSQNTLYF

SEQ ID NO: 643 - protein, 12 aa, Artificial sequence
  TCR-CDR3 peptide sequence
CASSTSAETLYF

SEQ ID NO: 644 - protein, 13 aa, Artificial sequence
  TCR-CDR3 peptide sequence
CASSWDNYAEQFF

SEQ ID NO: 645 - protein, 13 aa, Artificial sequence
  TCR-CDR3 peptide sequence
CASSWDSAETLYF

SEQ ID NO: 646 - protein, 12 aa, Artificial sequence
  TCR-CDR3 peptide sequence
CASSWGQNTLYF

SEQ ID NO: 647 - protein, 13 aa, Artificial sequence
  TCR-CDR3 peptide sequence
CASSWGSAETLYF

SEQ ID NO: 648 - protein, 12 aa, Artificial sequence
  TCR-CDR3 peptide sequence
CASSYSAETLYF

SEQ ID NO: 649 - protein, 12 aa, Artificial sequence
  TCR-CDR3 peptide sequence
CASSYSQNTLYF

SEQ ID NO: 650 - protein, 14 aa, Artificial sequence
  TCR-CDR3 peptide sequence
CAWSLQGYNSPLYF

SEQ ID NO: 651 - protein, 13 aa, Artificial sequence
  TCR-CDR3 peptide sequence
CASGDAGAETLYF

SEQ ID NO: 652 - protein, 12 aa, Artificial sequence
  TCR-CDR3 peptide sequence
CASGDAGDTQYF

SEQ ID NO: 653 - protein, 14 aa, Artificial sequence
  TCR-CDR3 peptide sequence
CASGDAGGQNTLYF

SEQ ID NO: 654 - protein, 14 aa, Artificial sequence
  TCR-CDR3 peptide sequence
CASGDAGGYAEQFF

SEQ ID NO: 655 - protein, 14 aa, Artificial sequence
  TCR-CDR3 peptide sequence
CASGDAGNTGQLYF

SEQ ID NO: 656 - protein, 14 aa, Artificial sequence
  TCR-CDR3 peptide sequence
CASGDAGQNTEVFF

SEQ ID NO: 657 - protein, 14 aa, Artificial sequence
  TCR-CDR3 peptide sequence
CASGDAGSAETLYF

SEQ ID NO: 658 - protein, 12 aa, Artificial sequence
  TCR-CDR3 peptide sequence
CASGDANSDYTF

SEQ ID NO: 659 - protein, 12 aa, Artificial sequence
  TCR-CDR3 peptide sequence
CASGDARDTQYF

SEQ ID NO: 660 - protein, 13 aa, Artificial sequence
  TCR-CDR3 peptide sequence
CASGDASAETLYF

SEQ ID NO: 661 - protein, 11 aa, Artificial sequence
  TCR-CDR3 peptide sequence
CASGDNTEVFF

SEQ ID NO: 662 - protein, 13 aa, Artificial sequence
  TCR-CDR3 peptide sequence
CASGDNYNSPLYF

SEQ ID NO: 663 - protein, 13 aa, Artificial sequence
  TCR-CDR3 peptide sequence
CASGDSSAETLYF

SEQ ID NO: 664 - protein, 14 aa, Artificial sequence
  TCR-CDR3 peptide sequence
CASGDWGNQDTQYF

SEQ ID NO: 665 - protein, 14 aa, Artificial sequence
  TCR-CDR3 peptide sequence
CASGDWGNYAEQFF

SEQ ID NO: 666 - protein, 10 aa, Artificial sequence
  TCR-CDR3 peptide sequence
CASGENTLYF

SEQ ID NO: 667 - protein, 13 aa, Artificial sequence
  TCR-CDR3 peptide sequence
CASGGQNYAEQFF

SEQ ID NO: 668 - protein, 12 aa, Artificial sequence
  TCR-CDR3 peptide sequence
CASGGSQNTLYF

SEQ ID NO: 669 - protein, 10 aa, Artificial sequence
  TCR-CDR3 peptide sequence
CASGQNTLYF

SEQ ID NO: 670 - protein, 13 aa, Artificial sequence
  TCR-CDR3 peptide sequence
CASKTANQDTQYF

SEQ ID NO: 671 - protein, 13 aa, Artificial sequence
  TCR-CDR3 peptide sequence
CASRDNSGNTLYF

SEQ ID NO: 672 - protein, 13 aa, Artificial sequence
  TCR-CDR3 peptide sequence
CASRDSSQNTLYF

SEQ ID NO: 673 - protein, 13 aa, Artificial sequence
  TCR-CDR3 peptide sequence
CASRRDSAETLYF

SEQ ID NO: 674 - protein, 13 aa, Artificial sequence
  TCR-CDR3 peptide sequence
CASRTGGYAEQFF

SEQ ID NO: 675 - protein, 12 aa, Artificial sequence
  TCR-CDR3 peptide sequence
CASRTSAETLYF

SEQ ID NO: 676 - protein, 12 aa, Artificial sequence
  TCR-CDR3 peptide sequence
CASSASAETLYF

SEQ ID NO: 677 - protein, 13 aa, Artificial sequence
  TCR-CDR3 peptide sequence
CASSDAGQNTLYF

SEQ ID NO: 678 - protein, 12 aa, Artificial sequence
  TCR-CDR3 peptide sequence
CASSDANSDYTF

SEQ ID NO: 679 - protein, 13 aa, Artificial sequence
  TCR-CDR3 peptide sequence
CASSDGSQNTLYF

SEQ ID NO: 680 - protein, 12 aa, Artificial sequence
  TCR-CDR3 peptide sequence
CASSDNQDTQYF

SEQ ID NO: 681 - protein, 12 aa, Artificial sequence
  TCR-CDR3 peptide sequence
CASSDNYAEQFF

SEQ ID NO: 682 - protein, 13 aa, Artificial sequence
  TCR-CDR3 peptide sequence
CASSDSSGNTLYF

SEQ ID NO: 683 - protein, 14 aa, Artificial sequence
  TCR-CDR3 peptide sequence
CASSDWGSAETLYF

SEQ ID NO: 684 - protein, 12 aa, Artificial sequence
  TCR-CDR3 peptide sequence
CASSESQNTLYF

SEQ ID NO: 685 - protein, 13 aa, Artificial sequence
  TCR-CDR3 peptide sequence
CASSFDSAETLYF

SEQ ID NO: 686 - protein, 11 aa, Artificial sequence
  TCR-CDR3 peptide sequence
CASSFDTEVFF

SEQ ID NO: 687 - protein, 13 aa, Artificial sequence
  TCR-CDR3 peptide sequence
CASSFGGQNTLYF

SEQ ID NO: 688 - protein, 11 aa, Artificial sequence
  TCR-CDR3 peptide sequence
CASSFQNTLYF

SEQ ID NO: 689 - protein, 13 aa, Artificial sequence
  TCR-CDR3 peptide sequence
CASSFSSAETLYF

SEQ ID NO: 690 - protein, 11 aa, Artificial sequence
  TCR-CDR3 peptide sequence
CASSGAETLYF

SEQ ID NO: 691 - protein, 13 aa, Artificial sequence
  TCR-CDR3 peptide sequence
CASSGQANTEVFF

SEQ ID NO: 692 - protein, 13 aa, Artificial sequence
  TCR-CDR3 peptide sequence
CASSGQNYAEQFF

SEQ ID NO: 693 - protein, 14 aa, Artificial sequence
  TCR-CDR3 peptide sequence
CASSGTASAETLYF

SEQ ID NO: 694 - protein, 14 aa, Artificial sequence
  TCR-CDR3 peptide sequence
CASSGTGGYAEQFF

SEQ ID NO: 695 - protein, 14 aa, Artificial sequence
  TCR-CDR3 peptide sequence
CASSGTISNERLFF

SEQ ID NO: 696 - protein, 14 aa, Artificial sequence
  TCR-CDR3 peptide sequence
CASSGTVSNERLFF

SEQ ID NO: 697 - protein, 11 aa, Artificial sequence
  TCR-CDR3 peptide sequence
CASSLAETLYF

SEQ ID NO: 698 - protein, 14 aa, Artificial sequence
  TCR-CDR3 peptide sequence
CASSLAGGYAEQFF

SEQ ID NO: 699 - protein, 14 aa, Artificial sequence
  TCR-CDR3 peptide sequence
CASSLAGSGNTLYF

SEQ ID NO: 700 - protein, 14 aa, Artificial sequence
  TCR-CDR3 peptide sequence
CASSLANSGNTLYF

SEQ ID NO: 701 - protein, 13 aa, Artificial sequence
  TCR-CDR3 peptide sequence
CASSLASQNTLYF

SEQ ID NO: 702 - protein, 12 aa, Artificial sequence
  TCR-CDR3 peptide sequence
CASSLDAETLYF

SEQ ID NO: 703 - protein, 13 aa, Artificial sequence
  TCR-CDR3 peptide sequence
CASSLDANSDYTF

SEQ ID NO: 704 - protein, 12 aa, Artificial sequence
  TCR-CDR3 peptide sequence
CASSLDNSDYTF

SEQ ID NO: 705 - protein, 14 aa, Artificial sequence
  TCR-CDR3 peptide sequence
CASSLDNSQNTLYF

SEQ ID NO: 706 - protein, 12 aa, Artificial sequence
  TCR-CDR3 peptide sequence
CASSLDNTEVFF

SEQ ID NO: 707 - protein, 12 aa, Artificial sequence
  TCR-CDR3 peptide sequence
CASSLDQNTLYF

SEQ ID NO: 708 - protein, 13 aa, Artificial sequence
  TCR-CDR3 peptide sequence
CASSLDRDTEVFF

SEQ ID NO: 709 - protein, 11 aa, Artificial sequence
  TCR-CDR3 peptide sequence
CASSLDREVFF

SEQ ID NO: 710 - protein, 14 aa, Artificial sequence
  TCR-CDR3 peptide sequence
CASSLDRGAETLYF

SEQ ID NO: 711 - protein, 13 aa, Artificial sequence
  TCR-CDR3 peptide sequence
CASSLDRNSDYTF

SEQ ID NO: 712 - protein, 13 aa, Artificial sequence
  TCR-CDR3 peptide sequence
CASSLDRNTEVFF

SEQ ID NO: 713 - protein, 14 aa, Artificial sequence
  TCR-CDR3 peptide sequence
CASSLDRNTGQLYF

SEQ ID NO: 714 - protein, 13 aa, Artificial sequence
  TCR-CDR3 peptide sequence
CASSLDTNSDYTF

SEQ ID NO: 715 - protein, 15 aa, Artificial sequence
  TCR-CDR3 peptide sequence
CASSLDWGNTGQLYF

SEQ ID NO: 716 - protein, 14 aa, Artificial sequence
  TCR-CDR3 peptide sequence
CASSLDWGQNTLYF

SEQ ID NO: 717 - protein, 15 aa, Artificial sequence
  TCR-CDR3 peptide sequence
CASSLDWGSQNTLYF

SEQ ID NO: 718 - protein, 14 aa, Artificial sequence
  TCR-CDR3 peptide sequence
CASSLDWGYAEQFF

SEQ ID NO: 719 - protein, 12 aa, Artificial sequence
  TCR-CDR3 peptide sequence
CASSLDYAEQFF

SEQ ID NO: 720 - protein, 13 aa, Artificial sequence
  TCR-CDR3 peptide sequence
CASSLEANTEVFF

SEQ ID NO: 721 - protein, 14 aa, Artificial sequence
  TCR-CDR3 peptide sequence
CASSLEGAGNTLYF

SEQ ID NO: 722 - protein, 14 aa, Artificial sequence
  TCR-CDR3 peptide sequence
CASSLEGGQNTLYF

SEQ ID NO: 723 - protein, 12 aa, Artificial sequence
  TCR-CDR3 peptide sequence
CASSLEGSDYTF

SEQ ID NO: 724 - protein, 13 aa, Artificial sequence
  TCR-CDR3 peptide sequence
CASSLEGTGQLYF

SEQ ID NO: 725 - protein, 12 aa, Artificial sequence
  TCR-CDR3 peptide sequence
CASSLENTEVFF

SEQ ID NO: 726 - protein, 11 aa, Artificial sequence
  TCR-CDR3 peptide sequence
CASSLENTLYF

SEQ ID NO: 727 - protein, 13 aa, Artificial sequence
  TCR-CDR3 peptide sequence
CASSLEQNSDYTF

SEQ ID NO: 728 - protein, 10 aa, Artificial sequence
  TCR-CDR3 peptide sequence
CASSLETLYF

SEQ ID NO: 729 - protein, 13 aa, Artificial sequence
  TCR-CDR3 peptide sequence
CASSLGANTEVFF

SEQ ID NO: 730 - protein, 14 aa, Artificial sequence
  TCR-CDR3 peptide sequence
CASSLGANTGQLYF

SEQ ID NO: 731 - protein, 12 aa, Artificial sequence
  TCR-CDR3 peptide sequence
CASSLGDTEVFF

SEQ ID NO: 732 - protein, 14 aa, Artificial sequence
  TCR-CDR3 peptide sequence
CASSLGGNTGQLYF

SEQ ID NO: 733 - protein, 14 aa, Artificial sequence
  TCR-CDR3 peptide sequence
CASSLGGNYAEQFF

SEQ ID NO: 734 - protein, 14 aa, Artificial sequence
  TCR-CDR3 peptide sequence
CASSLGGSGNTLYF

SEQ ID NO: 735 - protein, 12 aa, Artificial sequence
  TCR-CDR3 peptide sequence
CASSLGGTEVFF

SEQ ID NO: 736 - protein, 13 aa, Artificial sequence
  TCR-CDR3 peptide sequence
CASSLGGYAEQFF

SEQ ID NO: 737 - protein, 13 aa, Artificial sequence
  TCR-CDR3 peptide sequence
CASSLGNYAEQFF

SEQ ID NO: 738 - protein, 14 aa, Artificial sequence
  TCR-CDR3 peptide sequence
CASSLGQANTEVFF

SEQ ID NO: 739 - protein, 14 aa, Artificial sequence
  TCR-CDR3 peptide sequence
CASSLGQGAETLYF

SEQ ID NO: 740 - protein, 14 aa, Artificial sequence
  TCR-CDR3 peptide sequence
CASSLGQGNTEVFF

SEQ ID NO: 741 - protein, 14 aa, Artificial sequence
  TCR-CDR3 peptide sequence
CASSLGQGYAEQFF

SEQ ID NO: 742 - protein, 13 aa, Artificial sequence
  TCR-CDR3 peptide sequence
CASSLGQYAEQFF

SEQ ID NO: 743 - protein, 14 aa, Artificial sequence
  TCR-CDR3 peptide sequence
CASSLGTANTEVFF

SEQ ID NO: 744 - protein, 11 aa, Artificial sequence
  TCR-CDR3 peptide sequence
CASSLGTEVFF

SEQ ID NO: 745 - protein, 13 aa, Artificial sequence
  TCR-CDR3 peptide sequence
CASSLGTNTEVFF

SEQ ID NO: 746 - protein, 14 aa, Artificial sequence
  TCR-CDR3 peptide sequence
CASSLGVNQDTQYF

SEQ ID NO: 747 - protein, 13 aa, Artificial sequence
  TCR-CDR3 peptide sequence
CASSLGVYAEQFF

SEQ ID NO: 748 - protein, 14 aa, Artificial sequence
  TCR-CDR3 peptide sequence
CASSLLGGYAEQFF

SEQ ID NO: 749 - protein, 11 aa, Artificial sequence
  TCR-CDR3 peptide sequence
CASSLNSDYTF

SEQ ID NO: 750 - protein, 13 aa, Artificial sequence
  TCR-CDR3 peptide sequence
CASSLNSQNTLYF

SEQ ID NO: 751 - protein, 11 aa, Artificial sequence
  TCR-CDR3 peptide sequence
CASSLNTEVFF

SEQ ID NO: 752 - protein, 14 aa, Artificial sequence
  TCR-CDR3 peptide sequence
CASSLQGANSDYTF

SEQ ID NO: 753 - protein, 12 aa, Artificial sequence
  TCR-CDR3 peptide sequence
CASSLQGNTLYF

SEQ ID NO: 754 - protein, 14 aa, Artificial sequence
  TCR-CDR3 peptide sequence
CASSLQGNYAEQFF

SEQ ID NO: 755 - protein, 13 aa, Artificial sequence
  TCR-CDR3 peptide sequence
CASSLQGQNTLYF

SEQ ID NO: 756 - protein, 13 aa, Artificial sequence
  TCR-CDR3 peptide sequence
CASSLQGYAEQFF

SEQ ID NO: 757 - protein, 12 aa, Artificial sequence
  TCR-CDR3 peptide sequence
CASSLQNSDYTF

SEQ ID NO: 758 - protein, 12 aa, Artificial sequence
  TCR-CDR3 peptide sequence
CASSLQNTEVFF

SEQ ID NO: 759 - protein, 13 aa, Artificial sequence
  TCR-CDR3 peptide sequence
CASSLRGQNTLYF

SEQ ID NO: 760 - protein, 13 aa, Artificial sequence
  TCR-CDR3 peptide sequence
CASSLRSQNTLYF

SEQ ID NO: 761 - protein, 14 aa, Artificial sequence
  TCR-CDR3 peptide sequence
CASSLSGGQNTLYF

SEQ ID NO: 762 - protein, 13 aa, Artificial sequence
  TCR-CDR3 peptide sequence
CASSLSSAETLYF

SEQ ID NO: 763 - protein, 14 aa, Artificial sequence
  TCR-CDR3 peptide sequence
CASSLTANTGQLYF

SEQ ID NO: 764 - protein, 13 aa, Artificial sequence
  TCR-CDR3 peptide sequence
CASSLTGAETLYF

SEQ ID NO: 765 - protein, 15 aa, Artificial sequence
  TCR-CDR3 peptide sequence
CASSLTGANTGQLYF

SEQ ID NO: 766 - protein, 14 aa, Artificial sequence
  TCR-CDR3 peptide sequence
CASSLTGGQNTLYF

SEQ ID NO: 767 - protein, 13 aa, Artificial sequence
  TCR-CDR3 peptide sequence
CASSLTTNTEVFF

SEQ ID NO: 768 - protein, 14 aa, Artificial sequence
  TCR-CDR3 peptide sequence
CASSLTTSAETLYF

SEQ ID NO: 769 - protein, 14 aa, Artificial sequence
  TCR-CDR3 peptide sequence
CASSLVGNQDTQYF

SEQ ID NO: 770 - protein, 14 aa, Artificial sequence
  TCR-CDR3 peptide sequence
CASSLVGSQNTLYF

SEQ ID NO: 771 - protein, 12 aa, Artificial sequence
  TCR-CDR3 peptide sequence
CASSNSAETLYF

SEQ ID NO: 772 - protein, 11 aa, Artificial sequence
  TCR-CDR3 peptide sequence
CASSPAETLYF

SEQ ID NO: 773 - protein, 13 aa, Artificial sequence
  TCR-CDR3 peptide sequence
CASSPDSNERLFF

SEQ ID NO: 774 - protein, 15 aa, Artificial sequence
  TCR-CDR3 peptide sequence
CASSPDWGSQNTLYF

SEQ ID NO: 775 - protein, 15 aa, Artificial sequence
  TCR-CDR3 peptide sequence
CASSPGAGSNERLFF

SEQ ID NO: 776 - protein, 15 aa, Artificial sequence
  TCR-CDR3 peptide sequence
CASSPGLGNYAEQFF

SEQ ID NO: 777 - protein, 14 aa, Artificial sequence
  TCR-CDR3 peptide sequence
CASSPGLGQDTQYF

SEQ ID NO: 778 - protein, 12 aa, Artificial sequence
  TCR-CDR3 peptide sequence
CASSPGNTEVFF

SEQ ID NO: 779 - protein, 14 aa, Artificial sequence
  TCR-CDR3 peptide sequence
CASSPGQGAETLYF

SEQ ID NO: 780 - protein, 14 aa, Artificial sequence
  TCR-CDR3 peptide sequence
CASSPGQNTGQLYF

SEQ ID NO: 781 - protein, 13 aa, Artificial sequence
  TCR-CDR3 peptide sequence
CASSPGSAETLYF

SEQ ID NO: 782 - protein, 15 aa, Artificial sequence
  TCR-CDR3 peptide sequence
CASSPGTANTGQLYF

SEQ ID NO: 783 - protein, 14 aa, Artificial sequence
  TCR-CDR3 peptide sequence
CASSPGTTNSDYTF

SEQ ID NO: 784 - protein, 13 aa, Artificial sequence
  TCR-CDR3 peptide sequence
CASSPQGAETLYF

SEQ ID NO: 785 - protein, 14 aa, Artificial sequence
  TCR-CDR3 peptide sequence
CASSPQGSAETLYF

SEQ ID NO: 786 - protein, 13 aa, Artificial sequence
  TCR-CDR3 peptide sequence
CASSPTANTEVFF

SEQ ID NO: 787 - protein, 14 aa, Artificial sequence
  TCR-CDR3 peptide sequence
CASSPTGGQNTLYF

SEQ ID NO: 788 - protein, 13 aa, Artificial sequence
  TCR-CDR3 peptide sequence
CASSQDSAETLYF

SEQ ID NO: 789 - protein, 14 aa, Artificial sequence
  TCR-CDR3 peptide sequence
CASSQDSSGNTLYF

SEQ ID NO: 790 - protein, 14 aa, Artificial sequence
  TCR-CDR3 peptide sequence
CASSQDSSQNTLYF

SEQ ID NO: 791 - protein, 15 aa, Artificial sequence
  TCR-CDR3 peptide sequence
CASSQDWGNYAEQFF

SEQ ID NO: 792 - protein, 13 aa, Artificial sequence
  TCR-CDR3 peptide sequence
CASSQGAGNTLYF

SEQ ID NO: 793 - protein, 10 aa, Artificial sequence
  TCR-CDR3 peptide sequence
CASSQNTLYF

SEQ ID NO: 794 - protein, 13 aa, Artificial sequence
  TCR-CDR3 peptide sequence
CASSQQGNTEVFF

SEQ ID NO: 795 - protein, 13 aa, Artificial sequence
  TCR-CDR3 peptide sequence
CASSQTSAETLYF

SEQ ID NO: 796 - protein, 11 aa, Artificial sequence
  TCR-CDR3 peptide sequence
CASSRAETLYF

SEQ ID NO: 797 - protein, 12 aa, Artificial sequence
  TCR-CDR3 peptide sequence
CASSRDKNTLYF

SEQ ID NO: 798 - protein, 13 aa, Artificial sequence
  TCR-CDR3 peptide sequence
CASSRDNQNTLYF

SEQ ID NO: 799 - protein, 12 aa, Artificial sequence
  TCR-CDR3 peptide sequence
CASSRDNSDYTF

SEQ ID NO: 800 - protein, 14 aa, Artificial sequence
  TCR-CDR3 peptide sequence
CASSRDNSQNTLYF

SEQ ID NO: 801 - protein, 13 aa, Artificial sequence
  TCR-CDR3 peptide sequence
CASSRDRDAEQFF

SEQ ID NO: 802 - protein, 11 aa, Artificial sequence
  TCR-CDR3 peptide sequence
CASSRDREVFF

SEQ ID NO: 803 - protein, 13 aa, Artificial sequence
  TCR-CDR3 peptide sequence
CASSRDRGNTLYF

SEQ ID NO: 804 - protein, 13 aa, Artificial sequence
  TCR-CDR3 peptide sequence
CASSRDRNTEVFF

SEQ ID NO: 805 - protein, 13 aa, Artificial sequence
  TCR-CDR3 peptide sequence
CASSRDSNERLFF

SEQ ID NO: 806 - protein, 14 aa, Artificial sequence
  TCR-CDR3 peptide sequence
CASSRDSNYAEQFF

SEQ ID NO: 807 - protein, 14 aa, Artificial sequence
  TCR-CDR3 peptide sequence
CASSRDSSQNTLYF

SEQ ID NO: 808 - protein, 13 aa, Artificial sequence
  TCR-CDR3 peptide sequence
CASSRDWGNTLYF

SEQ ID NO: 809 - protein, 15 aa, Artificial sequence
  TCR-CDR3 peptide sequence
CASSRDWGSQNTLYF

SEQ ID NO: 810 - protein, 14 aa, Artificial sequence
  TCR-CDR3 peptide sequence
CASSRDWGYAEQFF

SEQ ID NO: 811 - protein, 13 aa, Artificial sequence
  TCR-CDR3 peptide sequence
CASSRGSQNTLYF

SEQ ID NO: 812 - protein, 14 aa, Artificial sequence
  TCR-CDR3 peptide sequence
CASSRLGNYAEQFF

SEQ ID NO: 813 - protein, 13 aa, Artificial sequence
  TCR-CDR3 peptide sequence
CASSRQGAETLYF

SEQ ID NO: 814 - protein, 12 aa, Artificial sequence
  TCR-CDR3 peptide sequence
CASSRQGNTLYF

SEQ ID NO: 815 - protein, 13 aa, Artificial sequence
  TCR-CDR3 peptide sequence
CASSRQGQNTLYF

SEQ ID NO: 816 - protein, 13 aa, Artificial sequence
  TCR-CDR3 peptide sequence
CASSRQNYAEQFF

SEQ ID NO: 817 - protein, 13 aa, Artificial sequence
  TCR-CDR3 peptide sequence
CASSRTGENTLYF

SEQ ID NO: 818 - protein, 13 aa, Artificial sequence
  TCR-CDR3 peptide sequence
CASSRTGQNTLYF

SEQ ID NO: 819 - protein, 13 aa, Artificial sequence
  TCR-CDR3 peptide sequence
CASSSGGQNTLYF

SEQ ID NO: 820 - protein, 11 aa, Artificial sequence
  TCR-CDR3 peptide sequence
CASSSGNTLYF

SEQ ID NO: 821 - protein, 13 aa, Artificial sequence
  TCR-CDR3 peptide sequence
CASSSGNYAEQFF

SEQ ID NO: 822 - protein, 13 aa, Artificial sequence
  TCR-CDR3 peptide sequence
CASSSGQQNTLYF

SEQ ID NO: 823 - protein, 14 aa, Artificial sequence
  TCR-CDR3 peptide sequence
CASSSGTTNTEVFF

SEQ ID NO: 824 - protein, 14 aa, Artificial sequence
  TCR-CDR3 peptide sequence
CASSSQGANTEVFF

SEQ ID NO: 825 - protein, 13 aa, Artificial sequence
  TCR-CDR3 peptide sequence
CASSSTANSDYTF

SEQ ID NO: 826 - protein, 13 aa, Artificial sequence
  TCR-CDR3 peptide sequence
CASSWGSQNTLYF

SEQ ID NO: 827 - protein, 12 aa, Artificial sequence
  TCR-CDR3 peptide sequence
CASSYGQNTLYF

SEQ ID NO: 828 - protein, 12 aa, Artificial sequence
  TCR-CDR3 peptide sequence
CGARDSAETLYF

SEQ ID NO: 829 - protein, 11 aa, Artificial sequence
  TCR-CDR3 peptide sequence
CSAGGQNTLYF

SEQ ID NO: 830 - protein, 10 aa, Artificial sequence
  TCR-CDR3 peptide sequence
CASGAETLYF

SEQ ID NO: 831 - protein, 14 aa, Artificial sequence
  TCR-CDR3 peptide sequence
CASGDADSGNTLYF

SEQ ID NO: 832 - protein, 11 aa, Artificial sequence
  TCR-CDR3 peptide sequence
CASGDAETLYF

SEQ ID NO: 833 - protein, 14 aa, Artificial sequence
  TCR-CDR3 peptide sequence
CASGDAGDQDTQYF

SEQ ID NO: 834 - protein, 11 aa, Artificial sequence
  TCR-CDR3 peptide sequence
CASGDAGEQYF

SEQ ID NO: 835 - protein, 13 aa, Artificial sequence
  TCR-CDR3 peptide sequence
CASGDAGGNTLYF

SEQ ID NO: 836 - protein, 13 aa, Artificial sequence
  TCR-CDR3 peptide sequence
CASGDAGNSDYTF

SEQ ID NO: 837 - protein, 14 aa, Artificial sequence
  TCR-CDR3 peptide sequence
CASGDANSGNTLYF

SEQ ID NO: 838 - protein, 13 aa, Artificial sequence
  TCR-CDR3 peptide sequence
CASGDASQNTLYF

SEQ ID NO: 839 - protein, 12 aa, Artificial sequence
  TCR-CDR3 peptide sequence
CASGDAYAEQFF

SEQ ID NO: 840 - protein, 13 aa, Artificial sequence
  TCR-CDR3 peptide sequence
CASGDNSGNTLYF

SEQ ID NO: 841 - protein, 14 aa, Artificial sequence
  TCR-CDR3 peptide sequence
CASGDPGNYAEQFF

SEQ ID NO: 842 - protein, 13 aa, Artificial sequence
  TCR-CDR3 peptide sequence
CASGDRGNTEVFF

SEQ ID NO: 843 - protein, 11 aa, Artificial sequence
  TCR-CDR3 peptide sequence
CASGDRYEQYF

SEQ ID NO: 844 - protein, 12 aa, Artificial sequence
  TCR-CDR3 peptide sequence
CASGDSQNTLYF

SEQ ID NO: 845 - protein, 13 aa, Artificial sequence
  TCR-CDR3 peptide sequence
CASGDSSGNTLYF

SEQ ID NO: 846 - protein, 12 aa, Artificial sequence
  TCR-CDR3 peptide sequence
CASGDSYAEQFF

SEQ ID NO: 847 - protein, 11 aa, Artificial sequence
  TCR-CDR3 peptide sequence
CASGDYAEQFF

SEQ ID NO: 848 - protein, 12 aa, Artificial sequence
  TCR-CDR3 peptide sequence
CASGEGQNTLYF

SEQ ID NO: 849 - protein, 12 aa, Artificial sequence
  TCR-CDR3 peptide sequence
CASGESAETLYF

SEQ ID NO: 850 - protein, 13 aa, Artificial sequence
  TCR-CDR3 peptide sequence
CASGGQANTEVFF

SEQ ID NO: 851 - protein, 14 aa, Artificial sequence
  TCR-CDR3 peptide sequence
CASRDWGNYAEQFF

SEQ ID NO: 852 - protein, 11 aa, Artificial sequence
  TCR-CDR3 peptide sequence
CASRGAETLYF

SEQ ID NO: 853 - protein, 12 aa, Artificial sequence
  TCR-CDR3 peptide sequence
CASSASQNTLYF

SEQ ID NO: 854 - protein, 12 aa, Artificial sequence
  TCR-CDR3 peptide sequence
CASSDAGNTLYF

SEQ ID NO: 855 - protein, 14 aa, Artificial sequence
  TCR-CDR3 peptide sequence
CASSDAGSQNTLYF

SEQ ID NO: 856 - protein, 13 aa, Artificial sequence
  TCR-CDR3 peptide sequence
CASSDASAETLYF

SEQ ID NO: 857 - protein, 12 aa, Artificial sequence
  TCR-CDR3 peptide sequence
CASSDRAETLYF

SEQ ID NO: 858 - protein, 12 aa, Artificial sequence
  TCR-CDR3 peptide sequence
CASSDRDTEVFF

SEQ ID NO: 859 - protein, 12 aa, Artificial sequence
  TCR-CDR3 peptide sequence
CASSDRNTEVFF

SEQ ID NO: 860 - protein, 13 aa, Artificial sequence
  TCR-CDR3 peptide sequence
CASSDWDQDTQYF

SEQ ID NO: 861 - protein, 14 aa, Artificial sequence
  TCR-CDR3 peptide sequence
CASSDWGNYAEQFF

SEQ ID NO: 862 - protein, 13 aa, Artificial sequence
  TCR-CDR3 peptide sequence
CASSDWGQNTLYF

SEQ ID NO: 863 - protein, 12 aa, Artificial sequence
  TCR-CDR3 peptide sequence
CASSFGAETLYF

SEQ ID NO: 864 - protein, 13 aa, Artificial sequence
  TCR-CDR3 peptide sequence
CASSFGGAETLYF

SEQ ID NO: 865 - protein, 13 aa, Artificial sequence
  TCR-CDR3 peptide sequence
CASSFGSAETLYF

SEQ ID NO: 866 - protein, 13 aa, Artificial sequence
  TCR-CDR3 peptide sequence
CASSFPSGNTLYF

SEQ ID NO: 867 - protein, 13 aa, Artificial sequence
  TCR-CDR3 peptide sequence
CASSFQGNTEVFF

SEQ ID NO: 868 - protein, 14 aa, Artificial sequence
  TCR-CDR3 peptide sequence
CASSFSGAQDTQYF

SEQ ID NO: 869 - protein, 13 aa, Artificial sequence
  TCR-CDR3 peptide sequence
CASSGDSQNTLYF

SEQ ID NO: 870 - protein, 13 aa, Artificial sequence
  TCR-CDR3 peptide sequence
CASSGDSYAEQFF

SEQ ID NO: 871 - protein, 14 aa, Artificial sequence
  TCR-CDR3 peptide sequence
CASSGLGNYAEQFF

SEQ ID NO: 872 - protein, 13 aa, Artificial sequence
  TCR-CDR3 peptide sequence
CASSGQGAETLYF

SEQ ID NO: 873 - protein, 14 aa, Artificial sequence
  TCR-CDR3 peptide sequence
CASSGTANTGQLYF

SEQ ID NO: 874 - protein, 13 aa, Artificial sequence
  TCR-CDR3 peptide sequence
CASSGTSNSDYTF

SEQ ID NO: 875 - protein, 14 aa, Artificial sequence
  TCR-CDR3 peptide sequence
CASSLAANTGQLYF

SEQ ID NO: 876 - protein, 14 aa, Artificial sequence
  TCR-CDR3 peptide sequence
CASSLAGDTGQLYF

SEQ ID NO: 877 - protein, 14 aa, Artificial sequence
  TCR-CDR3 peptide sequence
CASSLAGNQDTQYF

SEQ ID NO: 878 - protein, 13 aa, Artificial sequence
  TCR-CDR3 peptide sequence
CASSLAGNTEVFF

SEQ ID NO: 879 - protein, 14 aa, Artificial sequence
  TCR-CDR3 peptide sequence
CASSLAGNTGQLYF

SEQ ID NO: 880 - protein, 14 aa, Artificial sequence
  TCR-CDR3 peptide sequence
CASSLAGSAETLYF

SEQ ID NO: 881 - protein, 13 aa, Artificial sequence
  TCR-CDR3 peptide sequence
CASSLDANTEVFF

SEQ ID NO: 882 - protein, 12 aa, Artificial sequence
  TCR-CDR3 peptide sequence
CASSLDGNTLYF

SEQ ID NO: 883 - protein, 13 aa, Artificial sequence
  TCR-CDR3 peptide sequence
CASSLDIYAEQFF

SEQ ID NO: 884 - protein, 12 aa, Artificial sequence
  TCR-CDR3 peptide sequence
CASSLDNERLFF

SEQ ID NO: 885 - protein, 14 aa, Artificial sequence
  TCR-CDR3 peptide sequence
CASSLDNNQDTQYF

SEQ ID NO: 886 - protein, 11 aa, Artificial sequence
  TCR-CDR3 peptide sequence
CASSLDQAPLF

SEQ ID NO: 887 - protein, 12 aa, Artificial sequence
  TCR-CDR3 peptide sequence
CASSLDRNTLYF

SEQ ID NO: 888 - protein, 13 aa, Artificial sequence
  TCR-CDR3 peptide sequence
CASSLDSGNTLYF

SEQ ID NO: 889 - protein, 14 aa, Artificial sequence
  TCR-CDR3 peptide sequence
CASSLDSNQDTQYF

SEQ ID NO: 890 - protein, 13 aa, Artificial sequence
  TCR-CDR3 peptide sequence
CASSLDSQDTQYF

SEQ ID NO: 891 - protein, 14 aa, Artificial sequence
  TCR-CDR3 peptide sequence
CASSLDTSQNTLYF

SEQ ID NO: 892 - protein, 13 aa, Artificial sequence
  TCR-CDR3 peptide sequence
CASSLDWGNTLYF

SEQ ID NO: 893 - protein, 14 aa, Artificial sequence
  TCR-CDR3 peptide sequence
CASSLEANTGQLYF

SEQ ID NO: 894 - protein, 13 aa, Artificial sequence
  TCR-CDR3 peptide sequence
CASSLEDTGQLYF

SEQ ID NO: 895 - protein, 14 aa, Artificial sequence
  TCR-CDR3 peptide sequence
CASSLEGANSDYTF

SEQ ID NO: 896 - protein, 13 aa, Artificial sequence
  TCR-CDR3 peptide sequence
CASSLEGQNTLYF

SEQ ID NO: 897 - protein, 14 aa, Artificial sequence
  TCR-CDR3 peptide sequence
CASSLEGSAETLYF

SEQ ID NO: 898 - protein, 13 aa, Artificial sequence
  TCR-CDR3 peptide sequence
CASSLENTGQLYF

SEQ ID NO: 899 - protein, 13 aa, Artificial sequence
  TCR-CDR3 peptide sequence
CASSLESAETLYF

SEQ ID NO: 900 - protein, 14 aa, Artificial sequence
  TCR-CDR3 peptide sequence
CASSLGASQNTLYF

SEQ ID NO: 901 - protein, 14 aa, Artificial sequence
  TCR-CDR3 peptide sequence
CASSLGDNYAEQFF

SEQ ID NO: 902 - protein, 14 aa, Artificial sequence
  TCR-CDR3 peptide sequence
CASSLGDSAETLYF

SEQ ID NO: 903 - protein, 14 aa, Artificial sequence
  TCR-CDR3 peptide sequence
CASSLGDSGNTLYF

SEQ ID NO: 904 - protein, 13 aa, Artificial sequence
  TCR-CDR3 peptide sequence
CASSLGDTGQLYF

SEQ ID NO: 905 - protein, 14 aa, Artificial sequence
  TCR-CDR3 peptide sequence
CASSLGGSNERLFF

SEQ ID NO: 906 - protein, 14 aa, Artificial sequence
  TCR-CDR3 peptide sequence
CASSLGQNTGQLYF

SEQ ID NO: 907 - protein, 14 aa, Artificial sequence
  TCR-CDR3 peptide sequence
CASSLGQSAETLYF

SEQ ID NO: 908 - protein, 13 aa, Artificial sequence
  TCR-CDR3 peptide sequence
CASSLGSGNTLYF

SEQ ID NO: 909 - protein, 12 aa, Artificial sequence
  TCR-CDR3 peptide sequence
CASSLGTGQLYF

SEQ ID NO: 910 - protein, 14 aa, Artificial sequence
  TCR-CDR3 peptide sequence
CASSLGTSAETLYF

SEQ ID NO: 911 - protein, 14 aa, Artificial sequence
  TCR-CDR3 peptide sequence
CASSLGTTNTEVFF

SEQ ID NO: 912 - protein, 14 aa, Artificial sequence
  TCR-CDR3 peptide sequence
CASSLGVSNERLFF

SEQ ID NO: 913 - protein, 14 aa, Artificial sequence
  TCR-CDR3 peptide sequence
CASSLLGDQDTQYF

SEQ ID NO: 914 - protein, 13 aa, Artificial sequence
  TCR-CDR3 peptide sequence
CASSLQGGNTLYF

SEQ ID NO: 915 - protein, 14 aa, Artificial sequence
  TCR-CDR3 peptide sequence
CASSLQGNQDTQYF

SEQ ID NO: 916 - protein, 14 aa, Artificial sequence
  TCR-CDR3 peptide sequence
CASSLQISNERLFF

SEQ ID NO: 917 - protein, 13 aa, Artificial sequence
  TCR-CDR3 peptide sequence
CASSLQSQNTLYF

SEQ ID NO: 918 - protein, 12 aa, Artificial sequence
  TCR-CDR3 peptide sequence
CASSLRAETLYF

SEQ ID NO: 919 - protein, 14 aa, Artificial sequence
  TCR-CDR3 peptide sequence
CASSLRDNQDTQYF

SEQ ID NO: 920 - protein, 13 aa, Artificial sequence
  TCR-CDR3 peptide sequence
CASSLRDTGQLYF

SEQ ID NO: 921 - protein, 13 aa, Artificial sequence
  TCR-CDR3 peptide sequence
CASSLRGNTEVFF

SEQ ID NO: 922 - protein, 14 aa, Artificial sequence
  TCR-CDR3 peptide sequence
CASSLRGSGNTLYF

SEQ ID NO: 923 - protein, 14 aa, Artificial sequence
  TCR-CDR3 peptide sequence
CASSLSGSAETLYF

SEQ ID NO: 924 - protein, 12 aa, Artificial sequence
  TCR-CDR3 peptide sequence
CASSLSNERLFF

SEQ ID NO: 925 - protein, 12 aa, Artificial sequence
  TCR-CDR3 peptide sequence
CASSLSTGQLYF

SEQ ID NO: 926 - protein, 14 aa, Artificial sequence
  TCR-CDR3 peptide sequence
CASSLTASQNTLYF

SEQ ID NO: 927 - protein, 14 aa, Artificial sequence
  TCR-CDR3 peptide sequence
CASSLTDSGNTLYF

SEQ ID NO: 928 - protein, 12 aa, Artificial sequence
  TCR-CDR3 peptide sequence
CASSLTENTLYF

SEQ ID NO: 929 - protein, 14 aa, Artificial sequence
  TCR-CDR3 peptide sequence
CASSLTGANTEVFF

SEQ ID NO: 930 - protein, 14 aa, Artificial sequence
  TCR-CDR3 peptide sequence
CASSLTGDQDTQYF

SEQ ID NO: 931 - protein, 13 aa, Artificial sequence
  TCR-CDR3 peptide sequence
CASSLTGGNTLYF

SEQ ID NO: 932 - protein, 15 aa, Artificial sequence
  TCR-CDR3 peptide sequence
CASSLTGGSQNTLYF

SEQ ID NO: 933 - protein, 12 aa, Artificial sequence
  TCR-CDR3 peptide sequence
CASSLTGNTLYF

SEQ ID NO: 934 - protein, 13 aa, Artificial sequence
  TCR-CDR3 peptide sequence
CASSLTGQNTLYF

SEQ ID NO: 935 - protein, 14 aa, Artificial sequence
  TCR-CDR3 peptide sequence
CASSLTGSAETLYF

SEQ ID NO: 936 - protein, 12 aa, Artificial sequence
  TCR-CDR3 peptide sequence
CASSLTNTEVFF

SEQ ID NO: 937 - protein, 14 aa, Artificial sequence
  TCR-CDR3 peptide sequence
CASSLTSSAETLYF

SEQ ID NO: 938 - protein, 14 aa, Artificial sequence
  TCR-CDR3 peptide sequence
CASSLVGNYAEQFF

SEQ ID NO: 939 - protein, 13 aa, Artificial sequence
  TCR-CDR3 peptide sequence
CASSLVNQDTQYF

SEQ ID NO: 940 - protein, 13 aa, Artificial sequence
  TCR-CDR3 peptide sequence
CASSLVSQNTLYF

SEQ ID NO: 941 - protein, 14 aa, Artificial sequence
  TCR-CDR3 peptide sequence
CASSLWGNYAEQFF

SEQ ID NO: 942 - protein, 14 aa, Artificial sequence
  TCR-CDR3 peptide sequence
CASSPDNSGNTLYF

SEQ ID NO: 943 - protein, 13 aa, Artificial sequence
  TCR-CDR3 peptide sequence
CASSPDSQNTLYF

SEQ ID NO: 944 - protein, 14 aa, Artificial sequence
  TCR-CDR3 peptide sequence
CASSPDWGQNTLYF

SEQ ID NO: 945 - protein, 12 aa, Artificial sequence
  TCR-CDR3 peptide sequence
CASSPGAETLYF

SEQ ID NO: 946 - protein, 14 aa, Artificial sequence
  TCR-CDR3 peptide sequence
CASSPGGNYAEQFF

SEQ ID NO: 947 - protein, 13 aa, Artificial sequence
  TCR-CDR3 peptide sequence
CASSPGQYAEQFF

SEQ ID NO: 948 - protein, 14 aa, Artificial sequence
  TCR-CDR3 peptide sequence
CASSPGQYNSPLYF

SEQ ID NO: 949 - protein, 15 aa, Artificial sequence
  TCR-CDR3 peptide sequence
CASSPGTSSAETLYF

SEQ ID NO: 950 - protein, 14 aa, Artificial sequence
  TCR-CDR3 peptide sequence
CASSPGTTNTEVFF

SEQ ID NO: 951 - protein, 15 aa, Artificial sequence
  TCR-CDR3 peptide sequence
CASSPGTTSAETLYF

SEQ ID NO: 952 - protein, 14 aa, Artificial sequence
  TCR-CDR3 peptide sequence
CASSPQANTGQLYF

SEQ ID NO: 953 - protein, 14 aa, Artificial sequence
  TCR-CDR3 peptide sequence
CASSPQGAGNTLYF

SEQ ID NO: 954 - protein, 14 aa, Artificial sequence
  TCR-CDR3 peptide sequence
CASSPQGNTGQLYF

SEQ ID NO: 955 - protein, 14 aa, Artificial sequence
  TCR-CDR3 peptide sequence
CASSPQGSQNTLYF

SEQ ID NO: 956 - protein, 14 aa, Artificial sequence
  TCR-CDR3 peptide sequence
CASSPTANTGQLYF

SEQ ID NO: 957 - protein, 15 aa, Artificial sequence
  TCR-CDR3 peptide sequence
CASSPTGGNYAEQFF

SEQ ID NO: 958 - protein, 14 aa, Artificial sequence
  TCR-CDR3 peptide sequence
CASSPTGNTGQLYF

SEQ ID NO: 959 - protein, 14 aa, Artificial sequence
  TCR-CDR3 peptide sequence
CASSPTGNYAEQFF

SEQ ID NO: 960 - protein, 13 aa, Artificial sequence
  TCR-CDR3 peptide sequence
CASSPTSAETLYF

SEQ ID NO: 961 - protein, 14 aa, Artificial sequence
  TCR-CDR3 peptide sequence
CASSPTSSQNTLYF

SEQ ID NO: 962 - protein, 13 aa, Artificial sequence
  TCR-CDR3 peptide sequence
CASSQDSQNTLYF

SEQ ID NO: 963 - protein, 15 aa, Artificial sequence
  TCR-CDR3 peptide sequence
CASSQDWGSQNTLYF

SEQ ID NO: 964 - protein, 13 aa, Artificial sequence
  TCR-CDR3 peptide sequence
CASSQGNYAEQFF

SEQ ID NO: 965 - protein, 13 aa, Artificial sequence
  TCR-CDR3 peptide sequence
CASSQGSAETLYF

SEQ ID NO: 966 - protein, 13 aa, Artificial sequence
  TCR-CDR3 peptide sequence
CASSQGSGNTLYF

SEQ ID NO: 967 - protein, 15 aa, Artificial sequence
  TCR-CDR3 peptide sequence
CASSQGTANTGQLYF

SEQ ID NO: 968 - protein, 11 aa, Artificial sequence
  TCR-CDR3 peptide sequence
CASSQNTEVFF

SEQ ID NO: 969 - protein, 14 aa, Artificial sequence
  TCR-CDR3 peptide sequence
CASSQQGANTEVFF

SEQ ID NO: 970 - protein, 14 aa, Artificial sequence
  TCR-CDR3 peptide sequence
CASSRDNSAETLYF

SEQ ID NO: 971 - protein, 14 aa, Artificial sequence
  TCR-CDR3 peptide sequence
CASSRDNSGNTLYF

SEQ ID NO: 972 - protein, 13 aa, Artificial sequence
  TCR-CDR3 peptide sequence
CASSRDRGAEQFF

SEQ ID NO: 973 - protein, 14 aa, Artificial sequence
  TCR-CDR3 peptide sequence
CASSRDRNTGQLYF

SEQ ID NO: 974 - protein, 14 aa, Artificial sequence
  TCR-CDR3 peptide sequence
CASSRDSSGNTLYF

SEQ ID NO: 975 - protein, 12 aa, Artificial sequence
  TCR-CDR3 peptide sequence
CASSRDTGQLYF

SEQ ID NO: 976 - protein, 15 aa, Artificial sequence
  TCR-CDR3 peptide sequence
CASSRDWGNTGQLYF

SEQ ID NO: 977 - protein, 13 aa, Artificial sequence
  TCR-CDR3 peptide sequence
CASSRLGQDTQYF

SEQ ID NO: 978 - protein, 12 aa, Artificial sequence
  TCR-CDR3 peptide sequence
CASSRNTGQLYF

SEQ ID NO: 979 - protein, 13 aa, Artificial sequence
  TCR-CDR3 peptide sequence
CASSRQANSDYTF

SEQ ID NO: 980 - protein, 14 aa, Artificial sequence
  TCR-CDR3 peptide sequence
CASSRQGANTEVFF

SEQ ID NO: 981 - protein, 15 aa, Artificial sequence
  TCR-CDR3 peptide sequence
CASSRQGANTGQLYF

SEQ ID NO: 982 - protein, 13 aa, Artificial sequence
  TCR-CDR3 peptide sequence
CASSRQNTGQLYF

SEQ ID NO: 983 - protein, 14 aa, Artificial sequence
  TCR-CDR3 peptide sequence
CASSRTDSGNTLYF

SEQ ID NO: 984 - protein, 13 aa, Artificial sequence
  TCR-CDR3 peptide sequence
CASSRTSQNTLYF

SEQ ID NO: 985 - protein, 13 aa, Artificial sequence
  TCR-CDR3 peptide sequence
CASSSDSQNTLYF

SEQ ID NO: 986 - protein, 14 aa, Artificial sequence
  TCR-CDR3 peptide sequence
CASSSDWGQDTQYF

SEQ ID NO: 987 - protein, 12 aa, Artificial sequence
  TCR-CDR3 peptide sequence
CASSSGNTEVFF

SEQ ID NO: 988 - protein, 14 aa, Artificial sequence
  TCR-CDR3 peptide sequence
CASSSGQANTEVFF

SEQ ID NO: 989 - protein, 12 aa, Artificial sequence
  TCR-CDR3 peptide sequence
CASSSGQNTLYF

SEQ ID NO: 990 - protein, 13 aa, Artificial sequence
  TCR-CDR3 peptide sequence
CASSSGQQDTQYF

SEQ ID NO: 991 - protein, 13 aa, Artificial sequence
  TCR-CDR3 peptide sequence
CASSSGTNTEVFF

SEQ ID NO: 992 - protein, 13 aa, Artificial sequence
  TCR-CDR3 peptide sequence
CASSSQGNSDYTF

SEQ ID NO: 993 - protein, 13 aa, Artificial sequence
  TCR-CDR3 peptide sequence
CASSSQGNTEVFF

SEQ ID NO: 994 - protein, 14 aa, Artificial sequence
  TCR-CDR3 peptide sequence
CASSSQGNTGQLYF

SEQ ID NO: 995 - protein, 12 aa, Artificial sequence
  TCR-CDR3 peptide sequence
CASSSSGNTLYF

SEQ ID NO: 996 - protein, 13 aa, Artificial sequence
  TCR-CDR3 peptide sequence
CASSSTANTEVFF

SEQ ID NO: 997 - protein, 14 aa, Artificial sequence
  TCR-CDR3 peptide sequence
CASSSTASQNTLYF

SEQ ID NO: 998 - protein, 13 aa, Artificial sequence
  TCR-CDR3 peptide sequence
CASSSTGNTEVFF

SEQ ID NO: 999 - protein, 13 aa, Artificial sequence
  TCR-CDR3 peptide sequence
CASSSTSAETLYF

SEQ ID NO: 1000 - protein, 11 aa, Artificial sequence
  TCR-CDR3 peptide sequence
CASSSYAEQFF

SEQ ID NO: 1001 - protein, 13 aa, Artificial sequence
  TCR-CDR3 peptide sequence
CASSTGNTGQLYF

SEQ ID NO: 1002 - protein, 13 aa, Artificial sequence
  TCR-CDR3 peptide sequence
CASSWDSQNTLYF

SEQ ID NO: 1003 - protein, 13 aa, Artificial sequence
  TCR-CDR3 peptide sequence
CASSWDSYAEQFF

SEQ ID NO: 1004 - protein, 13 aa, Artificial sequence
  TCR-CDR3 peptide sequence
CASSWGNYAEQFF

SEQ ID NO: 1005 - protein, 11 aa, Artificial sequence
  TCR-CDR3 peptide sequence
CSKDSAETLYF

SEQ ID NO: 1006 - protein, 13 aa, Artificial sequence
  TCR-CDR3 peptide sequence
CSSSQGTNERLFF

SEQ ID NO: 1007 - protein, 11 aa, Artificial sequence
  TCR-CDR3 peptide sequence
CASGDADEQYF

SEQ ID NO: 1008 - protein, 13 aa, Artificial sequence
  TCR-CDR3 peptide sequence
CASGDADTGQLYF

SEQ ID NO: 1009 - protein, 12 aa, Artificial sequence
  TCR-CDR3 peptide sequence
CASGDAGAEQFF

SEQ ID NO: 1010 - protein, 14 aa, Artificial sequence
  TCR-CDR3 peptide sequence
CASGDAGANSDYTF

SEQ ID NO: 1011 - protein, 14 aa, Artificial sequence
  TCR-CDR3 peptide sequence
CASGDAGDTGQLYF

SEQ ID NO: 1012 - protein, 14 aa, Artificial sequence
  TCR-CDR3 peptide sequence
CASGDAGGAETLYF

SEQ ID NO: 1013 - protein, 14 aa, Artificial sequence
  TCR-CDR3 peptide sequence
CASGDAGNQDTQYF

SEQ ID NO: 1014 - protein, 13 aa, Artificial sequence
  TCR-CDR3 peptide sequence
CASGDAGNTEVFF

SEQ ID NO: 1015 - protein, 14 aa, Artificial sequence
  TCR-CDR3 peptide sequence
CASGDAGNYAEQFF

SEQ ID NO: 1016 - protein, 13 aa, Artificial sequence
  TCR-CDR3 peptide sequence
CASGDAGQDTQYF

SEQ ID NO: 1017 - protein, 14 aa, Artificial sequence
  TCR-CDR3 peptide sequence
CASGDAQSGNTLYF

SEQ ID NO: 1018 - protein, 12 aa, Artificial sequence
  TCR-CDR3 peptide sequence
CASGDDQDTQYF

SEQ ID NO: 1019 - protein, 13 aa, Artificial sequence
  TCR-CDR3 peptide sequence
CASGDGGQNTLYF

SEQ ID NO: 1020 - protein, 13 aa, Artificial sequence
  TCR-CDR3 peptide sequence
CASGDNSQNTLYF

SEQ ID NO: 1021 - protein, 13 aa, Artificial sequence
  TCR-CDR3 peptide sequence
CASGDPSAETLYF

SEQ ID NO: 1022 - protein, 13 aa, Artificial sequence
  TCR-CDR3 peptide sequence
CASGDPSQNTLYF

SEQ ID NO: 1023 - protein, 14 aa, Artificial sequence
  TCR-CDR3 peptide sequence
CASGDRGSGNTLYF

SEQ ID NO: 1024 - protein, 14 aa, Artificial sequence
  TCR-CDR3 peptide sequence
CASGDRGSQNTLYF

SEQ ID NO: 1025 - protein, 12 aa, Artificial sequence
  TCR-CDR3 peptide sequence
CASGDRNTEVFF

SEQ ID NO: 1026 - protein, 12 aa, Artificial sequence
  TCR-CDR3 peptide sequence
CASGDSNERLFF

SEQ ID NO: 1027 - protein, 13 aa, Artificial sequence
  TCR-CDR3 peptide sequence
CASGEGGQNTLYF

SEQ ID NO: 1028 - protein, 13 aa, Artificial sequence
  TCR-CDR3 peptide sequence
CASGGQGNTEVFF

SEQ ID NO: 1029 - protein, 13 aa, Artificial sequence
  TCR-CDR3 peptide sequence
CASGGTANTEVFF

SEQ ID NO: 1030 - protein, 11 aa, Artificial sequence
  TCR-CDR3 peptide sequence
CASGSAETLYF

SEQ ID NO: 1031 - protein, 12 aa, Artificial sequence
  TCR-CDR3 peptide sequence
CASRDNYAEQFF

SEQ ID NO: 1032 - protein, 13 aa, Artificial sequence
  TCR-CDR3 peptide sequence
CASRDSNYAEQFF

SEQ ID NO: 1033 - protein, 13 aa, Artificial sequence
  TCR-CDR3 peptide sequence
CASRDSSGNTLYF

SEQ ID NO: 1034 - protein, 14 aa, Artificial sequence
  TCR-CDR3 peptide sequence
CASRDWGSAETLYF

SEQ ID NO: 1035 - protein, 13 aa, Artificial sequence
  TCR-CDR3 peptide sequence
CASRGQNYAEQFF

SEQ ID NO: 1036 - protein, 14 aa, Artificial sequence
  TCR-CDR3 peptide sequence
CASSDAGGQNTLYF

SEQ ID NO: 1037 - protein, 12 aa, Artificial sequence
  TCR-CDR3 peptide sequence
CASSDANTEVFF

SEQ ID NO: 1038 - protein, 12 aa, Artificial sequence
  TCR-CDR3 peptide sequence
CASSDGAETLYF

SEQ ID NO: 1039 - protein, 14 aa, Artificial sequence
  TCR-CDR3 peptide sequence
CASSDGGNYAEQFF

SEQ ID NO: 1040 - protein, 13 aa, Artificial sequence
  TCR-CDR3 peptide sequence
CASSDNSGNTLYF

SEQ ID NO: 1041 - protein, 13 aa, Artificial sequence
  TCR-CDR3 peptide sequence
CASSDNSQNTLYF

SEQ ID NO: 1042 - protein, 11 aa, Artificial sequence
  TCR-CDR3 peptide sequence
CASSDNTEVFF

SEQ ID NO: 1043 - protein, 13 aa, Artificial sequence
  TCR-CDR3 peptide sequence
CASSDRGAETLYF

SEQ ID NO: 1044 - protein, 12 aa, Artificial sequence
  TCR-CDR3 peptide sequence
CASSDRNSDYTF

SEQ ID NO: 1045 - protein, 12 aa, Artificial sequence
  TCR-CDR3 peptide sequence
CASSEGQNTLYF

SEQ ID NO: 1046 - protein, 13 aa, Artificial sequence
  TCR-CDR3 peptide sequence
CASSEGSQNTLYF

SEQ ID NO: 1047 - protein, 13 aa, Artificial sequence
  TCR-CDR3 peptide sequence
CASSENSGNTLYF

SEQ ID NO: 1048 - protein, 12 aa, Artificial sequence
  TCR-CDR3 peptide sequence
CASSENTGQLYF

SEQ ID NO: 1049 - protein, 13 aa, Artificial sequence
  TCR-CDR3 peptide sequence
CASSETANTEVFF

SEQ ID NO: 1050 - protein, 14 aa, Artificial sequence
  TCR-CDR3 peptide sequence
CASSFGGNYAEQFF

SEQ ID NO: 1051 - protein, 14 aa, Artificial sequence
  TCR-CDR3 peptide sequence
CASSFGGSAETLYF

SEQ ID NO: 1052 - protein, 14 aa, Artificial sequence
  TCR-CDR3 peptide sequence
CASSFGTANTEVFF

SEQ ID NO: 1053 - protein, 12 aa, Artificial sequence
  TCR-CDR3 peptide sequence
CASSFNYAEQFF

SEQ ID NO: 1054 - protein, 13 aa, Artificial sequence
  TCR-CDR3 peptide sequence
CASSGQGNTEVFF

SEQ ID NO: 1055 - protein, 13 aa, Artificial sequence
  TCR-CDR3 peptide sequence
CASSGQGQNTLYF

SEQ ID NO: 1056 - protein, 12 aa, Artificial sequence
  TCR-CDR3 peptide sequence
CASSGSAETLYF

SEQ ID NO: 1057 - protein, 12 aa, Artificial sequence
  TCR-CDR3 peptide sequence
CASSGSQNTLYF

SEQ ID NO: 1058 - protein, 13 aa, Artificial sequence
  TCR-CDR3 peptide sequence
CASSGTGNSDYTF

SEQ ID NO: 1059 - protein, 13 aa, Artificial sequence
  TCR-CDR3 peptide sequence
CASSGTGQNTLYF

SEQ ID NO: 1060 - protein, 14 aa, Artificial sequence
  TCR-CDR3 peptide sequence
CASSGTTNTGQLYF

SEQ ID NO: 1061 - protein, 12 aa, Artificial sequence
  TCR-CDR3 peptide sequence
CASSINQDTQYF

SEQ ID NO: 1062 - protein, 13 aa, Artificial sequence
  TCR-CDR3 peptide sequence
CASSLAANTEVFF

SEQ ID NO: 1063 - protein, 13 aa, Artificial sequence
  TCR-CDR3 peptide sequence
CASSLAGAETLYF

SEQ ID NO: 1064 - protein, 14 aa, Artificial sequence
  TCR-CDR3 peptide sequence
CASSLAGDQDTQYF

SEQ ID NO: 1065 - protein, 13 aa, Artificial sequence
  TCR-CDR3 peptide sequence
CASSLAGENTLYF

SEQ ID NO: 1066 - protein, 13 aa, Artificial sequence
  TCR-CDR3 peptide sequence
CASSLAGQNTLYF

SEQ ID NO: 1067 - protein, 14 aa, Artificial sequence
  TCR-CDR3 peptide sequence
CASSLDGGQNTLYF

SEQ ID NO: 1068 - protein, 13 aa, Artificial sequence
  TCR-CDR3 peptide sequence
CASSLDGNTEVFF

SEQ ID NO: 1069 - protein, 13 aa, Artificial sequence
  TCR-CDR3 peptide sequence
CASSLDGYAEQFF

SEQ ID NO: 1070 - protein, 13 aa, Artificial sequence
  TCR-CDR3 peptide sequence
CASSLDNTGQLYF

SEQ ID NO: 1071 - protein, 14 aa, Artificial sequence
  TCR-CDR3 peptide sequence
CASSLDRAGNTLYF

SEQ ID NO: 1072 - protein, 14 aa, Artificial sequence
  TCR-CDR3 peptide sequence
CASSLDRANSDYTF

SEQ ID NO: 1073 - protein, 13 aa, Artificial sequence
  TCR-CDR3 peptide sequence
CASSLDRDAEQFF

SEQ ID NO: 1074 - protein, 12 aa, Artificial sequence
  TCR-CDR3 peptide sequence
CASSLDRGEVFF

SEQ ID NO: 1075 - protein, 12 aa, Artificial sequence
  TCR-CDR3 peptide sequence
CASSLDRTEVFF

SEQ ID NO: 1076 - protein, 14 aa, Artificial sequence
  TCR-CDR3 peptide sequence
CASSLEGANTEVFF

SEQ ID NO: 1077 - protein, 14 aa, Artificial sequence
  TCR-CDR3 peptide sequence
CASSLEGDQDTQYF

SEQ ID NO: 1078 - protein, 13 aa, Artificial sequence
  TCR-CDR3 peptide sequence
CASSLEGDSDYTF

SEQ ID NO: 1079 - protein, 14 aa, Artificial sequence
  TCR-CDR3 peptide sequence
CASSLEGDTGQLYF

SEQ ID NO: 1080 - protein, 15 aa, Artificial sequence
  TCR-CDR3 peptide sequence
CASSLEGSSAETLYF

SEQ ID NO: 1081 - protein, 15 aa, Artificial sequence
  TCR-CDR3 peptide sequence
CASSLEGTSAETLYF

SEQ ID NO: 1082 - protein, 12 aa, Artificial sequence
  TCR-CDR3 peptide sequence
CASSLENSDYTF

SEQ ID NO: 1083 - protein, 13 aa, Artificial sequence
  TCR-CDR3 peptide sequence
CASSLESQNTLYF

SEQ ID NO: 1084 - protein, 14 aa, Artificial sequence
  TCR-CDR3 peptide sequence
CASSLGGGQNTLYF

SEQ ID NO: 1085 - protein, 14 aa, Artificial sequence
  TCR-CDR3 peptide sequence
CASSLGGGYAEQFF

SEQ ID NO: 1086 - protein, 12 aa, Artificial sequence
  TCR-CDR3 peptide sequence
CASSLGGSDYTF

SEQ ID NO: 1087 - protein, 13 aa, Artificial sequence
  TCR-CDR3 peptide sequence
CASSLGHQDTQYF

SEQ ID NO: 1088 - protein, 14 aa, Artificial sequence
  TCR-CDR3 peptide sequence
CASSLGISNERLFF

SEQ ID NO: 1089 - protein, 14 aa, Artificial sequence
  TCR-CDR3 peptide sequence
CASSLGLGAETLYF

SEQ ID NO: 1090 - protein, 14 aa, Artificial sequence
  TCR-CDR3 peptide sequence
CASSLGLGYAEQFF

SEQ ID NO: 1091 - protein, 14 aa, Artificial sequence
  TCR-CDR3 peptide sequence
CASSLGNSAETLYF

SEQ ID NO: 1092 - protein, 13 aa, Artificial sequence
  TCR-CDR3 peptide sequence
CASSLGQGTEVFF

SEQ ID NO: 1093 - protein, 14 aa, Artificial sequence
  TCR-CDR3 peptide sequence
CASSLGSSQNTLYF

SEQ ID NO: 1094 - protein, 15 aa, Artificial sequence
  TCR-CDR3 peptide sequence
CASSLGTANTGQLYF

SEQ ID NO: 1095 - protein, 15 aa, Artificial sequence
  TCR-CDR3 peptide sequence
CASSLGTASAETLYF

SEQ ID NO: 1096 - protein, 15 aa, Artificial sequence
  TCR-CDR3 peptide sequence
CASSLGTSSAETLYF

SEQ ID NO: 1097 - protein, 14 aa, Artificial sequence
  TCR-CDR3 peptide sequence
CASSLGVNYAEQFF

SEQ ID NO: 1098 - protein, 14 aa, Artificial sequence
  TCR-CDR3 peptide sequence
CASSLGVSQNTLYF

SEQ ID NO: 1099 - protein, 15 aa, Artificial sequence
  TCR-CDR3 peptide sequence
CASSLLGANTGQLYF

SEQ ID NO: 1100 - protein, 13 aa, Artificial sequence
  TCR-CDR3 peptide sequence
CASSLPSAETLYF

SEQ ID NO: 1101 - protein, 15 aa, Artificial sequence
  TCR-CDR3 peptide sequence
CASSLQGANTGQLYF

SEQ ID NO: 1102 - protein, 14 aa, Artificial sequence
  TCR-CDR3 peptide sequence
CASSLQGDQDTQYF

SEQ ID NO: 1103 - protein, 12 aa, Artificial sequence
  TCR-CDR3 peptide sequence
CASSLQGSDYTF

SEQ ID NO: 1104 - protein, 12 aa, Artificial sequence
  TCR-CDR3 peptide sequence
CASSLQQDTQYF

SEQ ID NO: 1105 - protein, 13 aa, Artificial sequence
  TCR-CDR3 peptide sequence
CASSLRQNTEVFF

SEQ ID NO: 1106 - protein, 13 aa, Artificial sequence
  TCR-CDR3 peptide sequence
CASSLRSAETLYF

SEQ ID NO: 1107 - protein, 13 aa, Artificial sequence
  TCR-CDR3 peptide sequence
CASSLSGAETLYF

SEQ ID NO: 1108 - protein, 14 aa, Artificial sequence
  TCR-CDR3 peptide sequence
CASSLSGNYAEQFF

SEQ ID NO: 1109 - protein, 13 aa, Artificial sequence
  TCR-CDR3 peptide sequence
CASSLSGQNTLYF

SEQ ID NO: 1110 - protein, 14 aa, Artificial sequence
  TCR-CDR3 peptide sequence
CASSLSGSGNTLYF

SEQ ID NO: 1111 - protein, 14 aa, Artificial sequence
  TCR-CDR3 peptide sequence
CASSLSGSQNTLYF

SEQ ID NO: 1112 - protein, 12 aa, Artificial sequence
  TCR-CDR3 peptide sequence
CASSLSNTEVFF

SEQ ID NO: 1113 - protein, 14 aa, Artificial sequence
  TCR-CDR3 peptide sequence
CASSLTASAETLYF

SEQ ID NO: 1114 - protein, 14 aa, Artificial sequence
  TCR-CDR3 peptide sequence
CASSLTGDYAEQFF

SEQ ID NO: 1115 - protein, 13 aa, Artificial sequence
  TCR-CDR3 peptide sequence
CASSLTGENTLYF

SEQ ID NO: 1116 - protein, 14 aa, Artificial sequence
  TCR-CDR3 peptide sequence
CASSLTGGAETLYF

SEQ ID NO: 1117 - protein, 14 aa, Artificial sequence
  TCR-CDR3 peptide sequence
CASSLTGNTGQLYF

SEQ ID NO: 1118 - protein, 13 aa, Artificial sequence
  TCR-CDR3 peptide sequence
CASSLTSQNTLYF

SEQ ID NO: 1119 - protein, 13 aa, Artificial sequence
  TCR-CDR3 peptide sequence
CASSLVSAETLYF

SEQ ID NO: 1120 - protein, 11 aa, Artificial sequence
  TCR-CDR3 peptide sequence
CASSNTGQLYF

SEQ ID NO: 1121 - protein, 11 aa, Artificial sequence
  TCR-CDR3 peptide sequence
CASSNYAEQFF

SEQ ID NO: 1122 - protein, 13 aa, Artificial sequence
  TCR-CDR3 peptide sequence
CASSPDNYAEQFF

SEQ ID NO: 1123 - protein, 13 aa, Artificial sequence
  TCR-CDR3 peptide sequence
CASSPDRNTEVFF

SEQ ID NO: 1124 - protein, 14 aa, Artificial sequence
  TCR-CDR3 peptide sequence
CASSPDRSQNTLYF

SEQ ID NO: 1125 - protein, 14 aa, Artificial sequence
  TCR-CDR3 peptide sequence
CASSPDWGQDTQYF

SEQ ID NO: 1126 - protein, 13 aa, Artificial sequence
  TCR-CDR3 peptide sequence
CASSPGGAETLYF

SEQ ID NO: 1127 - protein, 14 aa, Artificial sequence
  TCR-CDR3 peptide sequence
CASSPGGSQNTLYF

SEQ ID NO: 1128 - protein, 12 aa, Artificial sequence
  TCR-CDR3 peptide sequence
CASSPGNSDYTF

SEQ ID NO: 1129 - protein, 14 aa, Artificial sequence
  TCR-CDR3 peptide sequence
CASSPGQANTEVFF

SEQ ID NO: 1130 - protein, 14 aa, Artificial sequence
  TCR-CDR3 peptide sequence
CASSPGQGQNTLYF

SEQ ID NO: 1131 - protein, 13 aa, Artificial sequence
  TCR-CDR3 peptide sequence
CASSPGQGTEVFF

SEQ ID NO: 1132 - protein, 14 aa, Artificial sequence
  TCR-CDR3 peptide sequence
CASSPGQNQDTQYF

SEQ ID NO: 1133 - protein, 13 aa, Artificial sequence
  TCR-CDR3 peptide sequence
CASSPGQNSDYTF

SEQ ID NO: 1134 - protein, 12 aa, Artificial sequence
  TCR-CDR3 peptide sequence
CASSPGQNTLYF

SEQ ID NO: 1135 - protein, 13 aa, Artificial sequence
  TCR-CDR3 peptide sequence
CASSPGQQNTLYF

SEQ ID NO: 1136 - protein, 12 aa, Artificial sequence
  TCR-CDR3 peptide sequence
CASSPGQTEVFF

SEQ ID NO: 1137 - protein, 13 aa, Artificial sequence
  TCR-CDR3 peptide sequence
CASSPGTAETLYF

SEQ ID NO: 1138 - protein, 14 aa, Artificial sequence
  TCR-CDR3 peptide sequence
CASSPGTANSDYTF

SEQ ID NO: 1139 - protein, 15 aa, Artificial sequence
  TCR-CDR3 peptide sequence
CASSPGTASAETLYF

SEQ ID NO: 1140 - protein, 14 aa, Artificial sequence
  TCR-CDR3 peptide sequence
CASSPGTGYAEQFF

SEQ ID NO: 1141 - protein, 13 aa, Artificial sequence
  TCR-CDR3 peptide sequence
CASSPGTNTEVFF

SEQ ID NO: 1142 - protein, 14 aa, Artificial sequence
  TCR-CDR3 peptide sequence
CASSPGTTNERLFF

SEQ ID NO: 1143 - protein, 14 aa, Artificial sequence
  TCR-CDR3 peptide sequence
CASSPLGSQNTLYF

SEQ ID NO: 1144 - protein, 14 aa, Artificial sequence
  TCR-CDR3 peptide sequence
CASSPTGGYAEQFF

SEQ ID NO: 1145 - protein, 14 aa, Artificial sequence
  TCR-CDR3 peptide sequence
CASSPTGNQDTQYF

SEQ ID NO: 1146 - protein, 14 aa, Artificial sequence
  TCR-CDR3 peptide sequence
CASSPTTSQNTLYF

SEQ ID NO: 1147 - protein, 14 aa, Artificial sequence
  TCR-CDR3 peptide sequence
CASSPTVSNERLFF

SEQ ID NO: 1148 - protein, 14 aa, Artificial sequence
  TCR-CDR3 peptide sequence
CASSPTVSQNTLYF

SEQ ID NO: 1149 - protein, 13 aa, Artificial sequence
  TCR-CDR3 peptide sequence
CASSQANTGQLYF

SEQ ID NO: 1150 - protein, 13 aa, Artificial sequence
  TCR-CDR3 peptide sequence
CASSQDSYAEQFF

SEQ ID NO: 1151 - protein, 14 aa, Artificial sequence
  TCR-CDR3 peptide sequence
CASSQEGSQNTLYF

SEQ ID NO: 1152 - protein, 12 aa, Artificial sequence
  TCR-CDR3 peptide sequence
CASSQGAETLYF

SEQ ID NO: 1153 - protein, 13 aa, Artificial sequence
  TCR-CDR3 peptide sequence
CASSQGGQNTLYF

SEQ ID NO: 1154 - protein, 13 aa, Artificial sequence
  TCR-CDR3 peptide sequence
CASSQGNTGQLYF

SEQ ID NO: 1155 - protein, 12 aa, Artificial sequence
  TCR-CDR3 peptide sequence
CASSQGQNTLYF

SEQ ID NO: 1156 - protein, 14 aa, Artificial sequence
  TCR-CDR3 peptide sequence
CASSQGQNYAEQFF

SEQ ID NO: 1157 - protein, 13 aa, Artificial sequence
  TCR-CDR3 peptide sequence
CASSQNSGNTLYF

SEQ ID NO: 1158 - protein, 13 aa, Artificial sequence
  TCR-CDR3 peptide sequence
CASSQQGAETLYF

SEQ ID NO: 1159 - protein, 14 aa, Artificial sequence
  TCR-CDR3 peptide sequence
CASSQQGNTGQLYF

SEQ ID NO: 1160 - protein, 14 aa, Artificial sequence
  TCR-CDR3 peptide sequence
CASSQQGSGNTLYF

SEQ ID NO: 1161 - protein, 14 aa, Artificial sequence
  TCR-CDR3 peptide sequence
CASSRDISQNTLYF

SEQ ID NO: 1162 - protein, 14 aa, Artificial sequence
  TCR-CDR3 peptide sequence
CASSRDNNYAEQFF

SEQ ID NO: 1163 - protein, 12 aa, Artificial sequence
  TCR-CDR3 peptide sequence
CASSRDNQAPLF

SEQ ID NO: 1164 - protein, 12 aa, Artificial sequence
  TCR-CDR3 peptide sequence
CASSRDNTEVFF

SEQ ID NO: 1165 - protein, 14 aa, Artificial sequence
  TCR-CDR3 peptide sequence
CASSRDRANSDYTF

SEQ ID NO: 1166 - protein, 12 aa, Artificial sequence
  TCR-CDR3 peptide sequence
CASSRDRNTLYF

SEQ ID NO: 1167 - protein, 14 aa, Artificial sequence
  TCR-CDR3 peptide sequence
CASSRDRNYAEQFF

SEQ ID NO: 1168 - protein, 14 aa, Artificial sequence
  TCR-CDR3 peptide sequence
CASSRDSANSDYTF

SEQ ID NO: 1169 - protein, 11 aa, Artificial sequence
  TCR-CDR3 peptide sequence
CASSRDTEVFF

SEQ ID NO: 1170 - protein, 12 aa, Artificial sequence
  TCR-CDR3 peptide sequence
CASSRGAETLYF

SEQ ID NO: 1171 - protein, 13 aa, Artificial sequence
  TCR-CDR3 peptide sequence
CASSRGNQDTQYF

SEQ ID NO: 1172 - protein, 13 aa, Artificial sequence
  TCR-CDR3 peptide sequence
CASSRGNYAEQFF

SEQ ID NO: 1173 - protein, 14 aa, Artificial sequence
  TCR-CDR3 peptide sequence
CASSRLGSQNTLYF

SEQ ID NO: 1174 - protein, 12 aa, Artificial sequence
  TCR-CDR3 peptide sequence
CASSRNQDTQYF

SEQ ID NO: 1175 - protein, 11 aa, Artificial sequence
  TCR-CDR3 peptide sequence
CASSRNTEVFF

SEQ ID NO: 1176 - protein, 14 aa, Artificial sequence
  TCR-CDR3 peptide sequence
CASSRQGAGNTLYF

SEQ ID NO: 1177 - protein, 13 aa, Artificial sequence
  TCR-CDR3 peptide sequence
CASSRQGDTEVFF

SEQ ID NO: 1178 - protein, 13 aa, Artificial sequence
  TCR-CDR3 peptide sequence
CASSRQSQNTLYF

SEQ ID NO: 1179 - protein, 13 aa, Artificial sequence
  TCR-CDR3 peptide sequence
CASSRTANSDYTF

SEQ ID NO: 1180 - protein, 14 aa, Artificial sequence
  TCR-CDR3 peptide sequence
CASSRTASAETLYF

SEQ ID NO: 1181 - protein, 14 aa, Artificial sequence
  TCR-CDR3 peptide sequence
CASSRTGGQNTLYF

SEQ ID NO: 1182 - protein, 14 aa, Artificial sequence
  TCR-CDR3 peptide sequence
CASSRTGGYAEQFF

SEQ ID NO: 1183 - protein, 13 aa, Artificial sequence
  TCR-CDR3 peptide sequence
CASSRTGNSDYTF

SEQ ID NO: 1184 - protein, 14 aa, Artificial sequence
  TCR-CDR3 peptide sequence
CASSRTGNTGQLYF

SEQ ID NO: 1185 - protein, 14 aa, Artificial sequence
  TCR-CDR3 peptide sequence
CASSRTGSQNTLYF

SEQ ID NO: 1186 - protein, 12 aa, Artificial sequence
  TCR-CDR3 peptide sequence
CASSRTNTEVFF

SEQ ID NO: 1187 - protein, 13 aa, Artificial sequence
  TCR-CDR3 peptide sequence
CASSRTTNTEVFF

SEQ ID NO: 1188 - protein, 11 aa, Artificial sequence
  TCR-CDR3 peptide sequence
CASSRYAEQFF

SEQ ID NO: 1189 - protein, 13 aa, Artificial sequence
  TCR-CDR3 peptide sequence
CASSSAANTEVFF

SEQ ID NO: 1190 - protein, 13 aa, Artificial sequence
  TCR-CDR3 peptide sequence
CASSSANTGQLYF

SEQ ID NO: 1191 - protein, 10 aa, Artificial sequence
  TCR-CDR3 peptide sequence
CASSSETLYF

SEQ ID NO: 1192 - protein, 12 aa, Artificial sequence
  TCR-CDR3 peptide sequence
CASSSGAETLYF

SEQ ID NO: 1193 - protein, 13 aa, Artificial sequence
  TCR-CDR3 peptide sequence
CASSSGGAETLYF

SEQ ID NO: 1194 - protein, 13 aa, Artificial sequence
  TCR-CDR3 peptide sequence
CASSSGGYAEQFF

SEQ ID NO: 1195 - protein, 13 aa, Artificial sequence
  TCR-CDR3 peptide sequence
CASSSGNQDTQYF

SEQ ID NO: 1196 - protein, 14 aa, Artificial sequence
  TCR-CDR3 peptide sequence
CASSSGQGAETLYF

SEQ ID NO: 1197 - protein, 14 aa, Artificial sequence
  TCR-CDR3 peptide sequence
CASSSGQGNTEVFF

SEQ ID NO: 1198 - protein, 13 aa, Artificial sequence
  TCR-CDR3 peptide sequence
CASSSGSAETLYF

SEQ ID NO: 1199 - protein, 14 aa, Artificial sequence
  TCR-CDR3 peptide sequence
CASSSGTGNTEVFF

SEQ ID NO: 1200 - protein, 11 aa, Artificial sequence
  TCR-CDR3 peptide sequence
CASSSNERLFF

SEQ ID NO: 1201 - protein, 12 aa, Artificial sequence
  TCR-CDR3 peptide sequence
CASSSNYAEQFF

SEQ ID NO: 1202 - protein, 13 aa, Artificial sequence
  TCR-CDR3 peptide sequence
CASSSQANTEVFF

SEQ ID NO: 1203 - protein, 14 aa, Artificial sequence
  TCR-CDR3 peptide sequence
CASSSQGSAETLYF

SEQ ID NO: 1204 - protein, 14 aa, Artificial sequence
  TCR-CDR3 peptide sequence
CASSSTGGYAEQFF

SEQ ID NO: 1205 - protein, 12 aa, Artificial sequence
  TCR-CDR3 peptide sequence
CASSTSQNTLYF

SEQ ID NO: 1206 - protein, 12 aa, Artificial sequence
  TCR-CDR3 peptide sequence
CASSWDNTEVFF

SEQ ID NO: 1207 - protein, 13 aa, Artificial sequence
  TCR-CDR3 peptide sequence
CASSWGNQDTQYF

SEQ ID NO: 1208 - protein, 13 aa, Artificial sequence
  TCR-CDR3 peptide sequence
CAWSLGSQNTLYF

SEQ ID NO: 1209 - protein, 13 aa, Artificial sequence
  TCR-CDR3 peptide sequence
CGARDSNYAEQFF

SEQ ID NO: 1210 - protein, 13 aa, Artificial sequence
  TCR-CDR3 peptide sequence
CGARDWGYAEQFF

SEQ ID NO: 1211 - protein, 12 aa, Artificial sequence
  TCR-CDR3 peptide sequence
CGARQGYAEQFF

SEQ ID NO: 1212 - protein, 11 aa, Artificial sequence
  TCR-CDR3 peptide sequence
CSAGSQNTLYF

SEQ ID NO: 1213 - protein, 10 aa, Artificial sequence
  TCR-CDR3 peptide sequence
CSASAETLYF

SEQ ID NO: 1214 - protein, 10 aa, Artificial sequence
  TCR-CDR3 peptide sequence
CSASQNTLYF

SEQ ID NO: 1215 - protein, 13 aa, Artificial sequence
  TCR-CDR3 peptide sequence
CASGDADQDTQYF

SEQ ID NO: 1216 - protein, 10 aa, Artificial sequence
  TCR-CDR3 peptide sequence
CASGDAEQYF

SEQ ID NO: 1217 - protein, 15 aa, Artificial sequence
  TCR-CDR3 peptide sequence
CASGDAGGNYAEQFF

SEQ ID NO: 1218 - protein, 14 aa, Artificial sequence
  TCR-CDR3 peptide sequence
CASGDAGSSYEQYF

SEQ ID NO: 1219 - protein, 12 aa, Artificial sequence
  TCR-CDR3 peptide sequence
CASGDANTEVFF

SEQ ID NO: 1220 - protein, 14 aa, Artificial sequence
  TCR-CDR3 peptide sequence
CASGDAPSQNTLYF

SEQ ID NO: 1221 - protein, 12 aa, Artificial sequence
  TCR-CDR3 peptide sequence
CASGDDAETLYF

SEQ ID NO: 1222 - protein, 14 aa, Artificial sequence
  TCR-CDR3 peptide sequence
CASGDGGNYAEQFF

SEQ ID NO: 1223 - protein, 13 aa, Artificial sequence
  TCR-CDR3 peptide sequence
CASGDRANTEVFF

SEQ ID NO: 1224 - protein, 13 aa, Artificial sequence
  TCR-CDR3 peptide sequence
CASGDRDQDTQYF

SEQ ID NO: 1225 - protein, 13 aa, Artificial sequence
  TCR-CDR3 peptide sequence
CASGDRGAETLYF

SEQ ID NO: 1226 - protein, 13 aa, Artificial sequence
  TCR-CDR3 peptide sequence
CASGDRNYAEQFF

SEQ ID NO: 1227 - protein, 12 aa, Artificial sequence
  TCR-CDR3 peptide sequence
CASGDTNSDYTF

SEQ ID NO: 1228 - protein, 10 aa, Artificial sequence
  TCR-CDR3 peptide sequence
CASGDVEQYF

SEQ ID NO: 1229 - protein, 14 aa, Artificial sequence
  TCR-CDR3 peptide sequence
CASGDWDSAETLYF

SEQ ID NO: 1230 - protein, 14 aa, Artificial sequence
  TCR-CDR3 peptide sequence
CASGDWGNTGQLYF

SEQ ID NO: 1231 - protein, 13 aa, Artificial sequence
  TCR-CDR3 peptide sequence
CASGDWGQNTLYF

SEQ ID NO: 1232 - protein, 13 aa, Artificial sequence
  TCR-CDR3 peptide sequence
CASGEGSQNTLYF

SEQ ID NO: 1233 - protein, 14 aa, Artificial sequence
  TCR-CDR3 peptide sequence
CASGETDSGNTLYF

SEQ ID NO: 1234 - protein, 13 aa, Artificial sequence
  TCR-CDR3 peptide sequence
CASGGTANSDYTF

SEQ ID NO: 1235 - protein, 12 aa, Artificial sequence
  TCR-CDR3 peptide sequence
CASGTTNTEVFF

SEQ ID NO: 1236 - protein, 13 aa, Artificial sequence
  TCR-CDR3 peptide sequence
CASRDRNYAEQFF

SEQ ID NO: 1237 - protein, 11 aa, Artificial sequence
  TCR-CDR3 peptide sequence
CASRNTGQLYF

SEQ ID NO: 1238 - protein, 13 aa, Artificial sequence
  TCR-CDR3 peptide sequence
CASRQGSQNTLYF

SEQ ID NO: 1239 - protein, 12 aa, Artificial sequence
  TCR-CDR3 peptide sequence
CASRWDNYEQYF

SEQ ID NO: 1240 - protein, 14 aa, Artificial sequence
  TCR-CDR3 peptide sequence
CASSDANSGNTLYF

SEQ ID NO: 1241 - protein, 13 aa, Artificial sequence
  TCR-CDR3 peptide sequence
CASSDASQNTLYF

SEQ ID NO: 1242 - protein, 14 aa, Artificial sequence
  TCR-CDR3 peptide sequence
CASSDDNSGNTLYF

SEQ ID NO: 1243 - protein, 13 aa, Artificial sequence
  TCR-CDR3 peptide sequence
CASSDGDTGQLYF

SEQ ID NO: 1244 - protein, 13 aa, Artificial sequence
  TCR-CDR3 peptide sequence
CASSDGGQNTLYF

SEQ ID NO: 1245 - protein, 12 aa, Artificial sequence
  TCR-CDR3 peptide sequence
CASSDNNERLFF

SEQ ID NO: 1246 - protein, 12 aa, Artificial sequence
  TCR-CDR3 peptide sequence
CASSDNNQAPLF

SEQ ID NO: 1247 - protein, 11 aa, Artificial sequence
  TCR-CDR3 peptide sequence
CASSDQNTLYF

SEQ ID NO: 1248 - protein, 13 aa, Artificial sequence
  TCR-CDR3 peptide sequence
CASSDRGNTEVFF

SEQ ID NO: 1249 - protein, 14 aa, Artificial sequence
  TCR-CDR3 peptide sequence
CASSDRGSGNTLYF

SEQ ID NO: 1250 - protein, 13 aa, Artificial sequence
  TCR-CDR3 peptide sequence
CASSDRNYAEQFF

SEQ ID NO: 1251 - protein, 12 aa, Artificial sequence
  TCR-CDR3 peptide sequence
CASSDSGNTLYF

SEQ ID NO: 1252 - protein, 12 aa, Artificial sequence
  TCR-CDR3 peptide sequence
CASSDSNERLFF

SEQ ID NO: 1253 - protein, 14 aa, Artificial sequence
  TCR-CDR3 peptide sequence
CASSDWGNQDTQYF

SEQ ID NO: 1254 - protein, 11 aa, Artificial sequence
  TCR-CDR3 peptide sequence
CASSDYAEQFF

SEQ ID NO: 1255 - protein, 12 aa, Artificial sequence
  TCR-CDR3 peptide sequence
CASSEGAETLYF

SEQ ID NO: 1256 - protein, 11 aa, Artificial sequence
  TCR-CDR3 peptide sequence
CASSFAETLYF

SEQ ID NO: 1257 - protein, 15 aa, Artificial sequence
  TCR-CDR3 peptide sequence
CASSFDWGNYAEQFF

SEQ ID NO: 1258 - protein, 14 aa, Artificial sequence
  TCR-CDR3 peptide sequence
CASSFGGSQNTLYF

SEQ ID NO: 1259 - protein, 12 aa, Artificial sequence
  TCR-CDR3 peptide sequence
CASSFNTGQLYF

SEQ ID NO: 1260 - protein, 12 aa, Artificial sequence
  TCR-CDR3 peptide sequence
CASSFRAETLYF

SEQ ID NO: 1261 - protein, 14 aa, Artificial sequence
  TCR-CDR3 peptide sequence
CASSFRGSQNTLYF

SEQ ID NO: 1262 - protein, 12 aa, Artificial sequence
  TCR-CDR3 peptide sequence
CASSFRNTEVFF

SEQ ID NO: 1263 - protein, 12 aa, Artificial sequence
  TCR-CDR3 peptide sequence
CASSFSGNTLYF

SEQ ID NO: 1264 - protein, 13 aa, Artificial sequence
  TCR-CDR3 peptide sequence
CASSGDNYAEQFF

SEQ ID NO: 1265 - protein, 13 aa, Artificial sequence
  TCR-CDR3 peptide sequence
CASSGDSAETLYF

SEQ ID NO: 1266 - protein, 14 aa, Artificial sequence
  TCR-CDR3 peptide sequence
CASSGDSSGNTLYF

SEQ ID NO: 1267 - protein, 13 aa, Artificial sequence
  TCR-CDR3 peptide sequence
CASSGQGNSDYTF

SEQ ID NO: 1268 - protein, 13 aa, Artificial sequence
  TCR-CDR3 peptide sequence
CASSGQSQNTLYF

SEQ ID NO: 1269 - protein, 12 aa, Artificial sequence
  TCR-CDR3 peptide sequence
CASSGTAETLYF

SEQ ID NO: 1270 - protein, 14 aa, Artificial sequence
  TCR-CDR3 peptide sequence
CASSGTGGAETLYF

SEQ ID NO: 1271 - protein, 15 aa, Artificial sequence
  TCR-CDR3 peptide sequence
CASSGTGGNYAEQFF

SEQ ID NO: 1272 - protein, 14 aa, Artificial sequence
  TCR-CDR3 peptide sequence
CASSGTGNYAEQFF

SEQ ID NO: 1273 - protein, 13 aa, Artificial sequence
  TCR-CDR3 peptide sequence
CASSGTTNTEVFF

SEQ ID NO: 1274 - protein, 13 aa, Artificial sequence
  TCR-CDR3 peptide sequence
CASSIGQNTEVFF

SEQ ID NO: 1275 - protein, 14 aa, Artificial sequence
  TCR-CDR3 peptide sequence
CASSIRDNYAEQFF

SEQ ID NO: 1276 - protein, 12 aa, Artificial sequence
  TCR-CDR3 peptide sequence
CASSISAETLYF

SEQ ID NO: 1277 - protein, 14 aa, Artificial sequence
  TCR-CDR3 peptide sequence
CASSLAGANTEVFF

SEQ ID NO: 1278 - protein, 12 aa, Artificial sequence
  TCR-CDR3 peptide sequence
CASSLAGNTLYF

SEQ ID NO: 1279 - protein, 12 aa, Artificial sequence
  TCR-CDR3 peptide sequence
CASSLANTEVFF

SEQ ID NO: 1280 - protein, 13 aa, Artificial sequence
  TCR-CDR3 peptide sequence
CASSLDGAETLYF

SEQ ID NO: 1281 - protein, 13 aa, Artificial sequence
  TCR-CDR3 peptide sequence
CASSLDGQNTLYF

SEQ ID NO: 1282 - protein, 15 aa, Artificial sequence
  TCR-CDR3 peptide sequence
CASSLDGSSAETLYF

SEQ ID NO: 1283 - protein, 12 aa, Artificial sequence
  TCR-CDR3 peptide sequence
CASSLDKNTLYF

SEQ ID NO: 1284 - protein, 13 aa, Artificial sequence
  TCR-CDR3 peptide sequence
CASSLDRDSDYTF

SEQ ID NO: 1285 - protein, 14 aa, Artificial sequence
  TCR-CDR3 peptide sequence
CASSLDRNYAEQFF

SEQ ID NO: 1286 - protein, 13 aa, Artificial sequence
  TCR-CDR3 peptide sequence
CASSLDSNERLFF

SEQ ID NO: 1287 - protein, 14 aa, Artificial sequence
  TCR-CDR3 peptide sequence
CASSLDSNYAEQFF

SEQ ID NO: 1288 - protein, 14 aa, Artificial sequence
  TCR-CDR3 peptide sequence
CASSLDSYNSPLYF

SEQ ID NO: 1289 - protein, 13 aa, Artificial sequence
  TCR-CDR3 peptide sequence
CASSLDTNTEVFF

SEQ ID NO: 1290 - protein, 13 aa, Artificial sequence
  TCR-CDR3 peptide sequence
CASSLEGDAEQFF

SEQ ID NO: 1291 - protein, 14 aa, Artificial sequence
  TCR-CDR3 peptide sequence
CASSLEGGYAEQFF

SEQ ID NO: 1292 - protein, 14 aa, Artificial sequence
  TCR-CDR3 peptide sequence
CASSLEGQQDTQYF

SEQ ID NO: 1293 - protein, 12 aa, Artificial sequence
  TCR-CDR3 peptide sequence
CASSLEGTEVFF

SEQ ID NO: 1294 - protein, 14 aa, Artificial sequence
  TCR-CDR3 peptide sequence
CASSLENSGNTLYF

SEQ ID NO: 1295 - protein, 13 aa, Artificial sequence
  TCR-CDR3 peptide sequence
CASSLENYAEQFF

SEQ ID NO: 1296 - protein, 12 aa, Artificial sequence
  TCR-CDR3 peptide sequence
CASSLEQTEVFF

SEQ ID NO: 1297 - protein, 13 aa, Artificial sequence
  TCR-CDR3 peptide sequence
CASSLGDYAEQFF

SEQ ID NO: 1298 - protein, 13 aa, Artificial sequence
  TCR-CDR3 peptide sequence
CASSLGENTEVFF

SEQ ID NO: 1299 - protein, 10 aa, Artificial sequence
  TCR-CDR3 peptide sequence
CASSLGEVFF

SEQ ID NO: 1300 - protein, 14 aa, Artificial sequence
  TCR-CDR3 peptide sequence
CASSLGGANTEVFF

SEQ ID NO: 1301 - protein, 13 aa, Artificial sequence
  TCR-CDR3 peptide sequence
CASSLGGTGQLYF

SEQ ID NO: 1302 - protein, 13 aa, Artificial sequence
  TCR-CDR3 peptide sequence
CASSLGLYAEQFF

SEQ ID NO: 1303 - protein, 12 aa, Artificial sequence
  TCR-CDR3 peptide sequence
CASSLGNQAPLF

SEQ ID NO: 1304 - protein, 14 aa, Artificial sequence
  TCR-CDR3 peptide sequence
CASSLGQGNSDYTF

SEQ ID NO: 1305 - protein, 13 aa, Artificial sequence
  TCR-CDR3 peptide sequence
CASSLGTENTLYF

SEQ ID NO: 1306 - protein, 14 aa, Artificial sequence
  TCR-CDR3 peptide sequence
CASSLGTGAETLYF

SEQ ID NO: 1307 - protein, 15 aa, Artificial sequence
  TCR-CDR3 peptide sequence
CASSLGTGNTGQLYF

SEQ ID NO: 1308 - protein, 13 aa, Artificial sequence
  TCR-CDR3 peptide sequence
CASSLGTNSDYTF

SEQ ID NO: 1309 - protein, 14 aa, Artificial sequence
  TCR-CDR3 peptide sequence
CASSLGTSQNTLYF

SEQ ID NO: 1310 - protein, 14 aa, Artificial sequence
  TCR-CDR3 peptide sequence
CASSLLGDYAEQFF

SEQ ID NO: 1311 - protein, 12 aa, Artificial sequence
  TCR-CDR3 peptide sequence
CASSLLGNTLYF

SEQ ID NO: 1312 - protein, 14 aa, Artificial sequence
  TCR-CDR3 peptide sequence
CASSLLGSQNTLYF

SEQ ID NO: 1313 - protein, 13 aa, Artificial sequence
  TCR-CDR3 peptide sequence
CASSLNNQDTQYF

SEQ ID NO: 1314 - protein, 13 aa, Artificial sequence
  TCR-CDR3 peptide sequence
CASSLNSGNTLYF

SEQ ID NO: 1315 - protein, 14 aa, Artificial sequence
  TCR-CDR3 peptide sequence
CASSLQANTGQLYF

SEQ ID NO: 1316 - protein, 13 aa, Artificial sequence
  TCR-CDR3 peptide sequence
CASSLQGDSDYTF

SEQ ID NO: 1317 - protein, 13 aa, Artificial sequence
  TCR-CDR3 peptide sequence
CASSLQGDTEVFF

SEQ ID NO: 1318 - protein, 12 aa, Artificial sequence
  TCR-CDR3 peptide sequence
CASSLQGETLYF

SEQ ID NO: 1319 - protein, 13 aa, Artificial sequence
  TCR-CDR3 peptide sequence
CASSLQGGAEQFF

SEQ ID NO: 1320 - protein, 14 aa, Artificial sequence
  TCR-CDR3 peptide sequence
CASSLQGSNERLFF

SEQ ID NO: 1321 - protein, 13 aa, Artificial sequence
  TCR-CDR3 peptide sequence
CASSLQGTGQLYF

SEQ ID NO: 1322 - protein, 14 aa, Artificial sequence
  TCR-CDR3 peptide sequence
CASSLQGYNSPLYF

SEQ ID NO: 1323 - protein, 13 aa, Artificial sequence
  TCR-CDR3 peptide sequence
CASSLQNYAEQFF

SEQ ID NO: 1324 - protein, 13 aa, Artificial sequence
  TCR-CDR3 peptide sequence
CASSLRGAETLYF

SEQ ID NO: 1325 - protein, 13 aa, Artificial sequence
  TCR-CDR3 peptide sequence
CASSLRGDTEVFF

SEQ ID NO: 1326 - protein, 14 aa, Artificial sequence
  TCR-CDR3 peptide sequence
CASSLRGNQDTQYF

SEQ ID NO: 1327 - protein, 14 aa, Artificial sequence
  TCR-CDR3 peptide sequence
CASSLRGNTGQLYF

SEQ ID NO: 1328 - protein, 12 aa, Artificial sequence
  TCR-CDR3 peptide sequence
CASSLRGNTLYF

SEQ ID NO: 1329 - protein, 14 aa, Artificial sequence
  TCR-CDR3 peptide sequence
CASSLRGNYAEQFF

SEQ ID NO: 1330 - protein, 14 aa, Artificial sequence
  TCR-CDR3 peptide sequence
CASSLRGSAETLYF

SEQ ID NO: 1331 - protein, 11 aa, Artificial sequence
  TCR-CDR3 peptide sequence
CASSLSETLYF

SEQ ID NO: 1332 - protein, 14 aa, Artificial sequence
  TCR-CDR3 peptide sequence
CASSLSGDQDTQYF

SEQ ID NO: 1333 - protein, 14 aa, Artificial sequence
  TCR-CDR3 peptide sequence
CASSLSGNQDTQYF

SEQ ID NO: 1334 - protein, 13 aa, Artificial sequence
  TCR-CDR3 peptide sequence
CASSLTGNSDYTF

SEQ ID NO: 1335 - protein, 14 aa, Artificial sequence
  TCR-CDR3 peptide sequence
CASSLTISNERLFF

SEQ ID NO: 1336 - protein, 12 aa, Artificial sequence
  TCR-CDR3 peptide sequence
CASSLTNSDYTF

SEQ ID NO: 1337 - protein, 14 aa, Artificial sequence
  TCR-CDR3 peptide sequence
CASSLTSSQNTLYF

SEQ ID NO: 1338 - protein, 14 aa, Artificial sequence
  TCR-CDR3 peptide sequence
CASSLTVSNERLFF

SEQ ID NO: 1339 - protein, 12 aa, Artificial sequence
  TCR-CDR3 peptide sequence
CASSLVAETLYF

SEQ ID NO: 1340 - protein, 14 aa, Artificial sequence
  TCR-CDR3 peptide sequence
CASSLVGDQDTQYF

SEQ ID NO: 1341 - protein, 14 aa, Artificial sequence
  TCR-CDR3 peptide sequence
CASSLVGGAETLYF

SEQ ID NO: 1342 - protein, 14 aa, Artificial sequence
  TCR-CDR3 peptide sequence
CASSLVGGQNTLYF

SEQ ID NO: 1343 - protein, 14 aa, Artificial sequence
  TCR-CDR3 peptide sequence
CASSLWGSAETLYF

SEQ ID NO: 1344 - protein, 12 aa, Artificial sequence
  TCR-CDR3 peptide sequence
CASSNSGNTLYF

SEQ ID NO: 1345 - protein, 12 aa, Artificial sequence
  TCR-CDR3 peptide sequence
CASSNSQNTLYF

SEQ ID NO: 1346 - protein, 14 aa, Artificial sequence
  TCR-CDR3 peptide sequence
CASSPDRGQNTLYF

SEQ ID NO: 1347 - protein, 14 aa, Artificial sequence
  TCR-CDR3 peptide sequence
CASSPDSNQDTQYF

SEQ ID NO: 1348 - protein, 13 aa, Artificial sequence
  TCR-CDR3 peptide sequence
CASSPDSYAEQFF

SEQ ID NO: 1349 - protein, 14 aa, Artificial sequence
  TCR-CDR3 peptide sequence
CASSPDWGAETLYF

SEQ ID NO: 1350 - protein, 15 aa, Artificial sequence
  TCR-CDR3 peptide sequence
CASSPDWGSAETLYF

SEQ ID NO: 1351 - protein, 14 aa, Artificial sequence
  TCR-CDR3 peptide sequence
CASSPDWGYAEQFF

SEQ ID NO: 1352 - protein, 12 aa, Artificial sequence
  TCR-CDR3 peptide sequence
CASSPDYAEQFF

SEQ ID NO: 1353 - protein, 13 aa, Artificial sequence
  TCR-CDR3 peptide sequence
CASSPGANTEVFF

SEQ ID NO: 1354 - protein, 14 aa, Artificial sequence
  TCR-CDR3 peptide sequence
CASSPGLGAETLYF

SEQ ID NO: 1355 - protein, 14 aa, Artificial sequence
  TCR-CDR3 peptide sequence
CASSPGLGENTLYF

SEQ ID NO: 1356 - protein, 14 aa, Artificial sequence
  TCR-CDR3 peptide sequence
CASSPGLGYAEQFF

SEQ ID NO: 1357 - protein, 14 aa, Artificial sequence
  TCR-CDR3 peptide sequence
CASSPGLNTGQLYF

SEQ ID NO: 1358 - protein, 14 aa, Artificial sequence
  TCR-CDR3 peptide sequence
CASSPGLNYAEQFF

SEQ ID NO: 1359 - protein, 13 aa, Artificial sequence
  TCR-CDR3 peptide sequence
CASSPGLQDTQYF

SEQ ID NO: 1360 - protein, 13 aa, Artificial sequence
  TCR-CDR3 peptide sequence
CASSPGNYAEQFF

SEQ ID NO: 1361 - protein, 13 aa, Artificial sequence
  TCR-CDR3 peptide sequence
CASSPGQNERLFF

SEQ ID NO: 1362 - protein, 14 aa, Artificial sequence
  TCR-CDR3 peptide sequence
CASSPGQSAETLYF

SEQ ID NO: 1363 - protein, 13 aa, Artificial sequence
  TCR-CDR3 peptide sequence
CASSPGTENTLYF

SEQ ID NO: 1364 - protein, 14 aa, Artificial sequence
  TCR-CDR3 peptide sequence
CASSPGTGAETLYF

SEQ ID NO: 1365 - protein, 14 aa, Artificial sequence
  TCR-CDR3 peptide sequence
CASSPGTSQNTLYF

SEQ ID NO: 1366 - protein, 14 aa, Artificial sequence
  TCR-CDR3 peptide sequence
CASSPLGNYAEQFF

SEQ ID NO: 1367 - protein, 11 aa, Artificial sequence
  TCR-CDR3 peptide sequence
CASSPNTEVFF

SEQ ID NO: 1368 - protein, 14 aa, Artificial sequence
  TCR-CDR3 peptide sequence
CASSPQGSGNTLYF

SEQ ID NO: 1369 - protein, 11 aa, Artificial sequence
  TCR-CDR3 peptide sequence
CASSPQNTLYF

SEQ ID NO: 1370 - protein, 12 aa, Artificial sequence
  TCR-CDR3 peptide sequence
CASSPRAETLYF

SEQ ID NO: 1371 - protein, 14 aa, Artificial sequence
  TCR-CDR3 peptide sequence
CASSPRDNYAEQFF

SEQ ID NO: 1372 - protein, 14 aa, Artificial sequence
  TCR-CDR3 peptide sequence
CASSPTASAETLYF

SEQ ID NO: 1373 - protein, 14 aa, Artificial sequence
  TCR-CDR3 peptide sequence
CASSPTGDQDTQYF

SEQ ID NO: 1374 - protein, 13 aa, Artificial sequence
  TCR-CDR3 peptide sequence
CASSPTSQNTLYF

SEQ ID NO: 1375 - protein, 12 aa, Artificial sequence
  TCR-CDR3 peptide sequence
CASSQANSDYTF

SEQ ID NO: 1376 - protein, 13 aa, Artificial sequence
  TCR-CDR3 peptide sequence
CASSQDNYAEQFF

SEQ ID NO: 1377 - protein, 13 aa, Artificial sequence
  TCR-CDR3 peptide sequence
CASSQDSGNTLYF

SEQ ID NO: 1378 - protein, 13 aa, Artificial sequence
  TCR-CDR3 peptide sequence
CASSQEGAETLYF

SEQ ID NO: 1379 - protein, 15 aa, Artificial sequence
  TCR-CDR3 peptide sequence
CASSQRDWCYAEQFF

SEQ ID NO: 1380 - protein, 12 aa, Artificial sequence
  TCR-CDR3 peptide sequence
CASSQSQNTLYF

SEQ ID NO: 1381 - protein, 13 aa, Artificial sequence
  TCR-CDR3 peptide sequence
CASSRANTGQLYF

SEQ ID NO: 1382 - protein, 14 aa, Artificial sequence
  TCR-CDR3 peptide sequence
CASSRDISAETLYF

SEQ ID NO: 1383 - protein, 14 aa, Artificial sequence
  TCR-CDR3 peptide sequence
CASSRDNNNQAPLF

SEQ ID NO: 1384 - protein, 13 aa, Artificial sequence
  TCR-CDR3 peptide sequence
CASSRDRAETLYF

SEQ ID NO: 1385 - protein, 14 aa, Artificial sequence
  TCR-CDR3 peptide sequence
CASSRDRGAETLYF

SEQ ID NO: 1386 - protein, 14 aa, Artificial sequence
  TCR-CDR3 peptide sequence
CASSRDRGNSDYTF

SEQ ID NO: 1387 - protein, 14 aa, Artificial sequence
  TCR-CDR3 peptide sequence
CASSRDRGQNTLYF

SEQ ID NO: 1388 - protein, 14 aa, Artificial sequence
  TCR-CDR3 peptide sequence
CASSRDRGYAEQFF

SEQ ID NO: 1389 - protein, 13 aa, Artificial sequence
  TCR-CDR3 peptide sequence
CASSRDSGNTLYF

SEQ ID NO: 1390 - protein, 13 aa, Artificial sequence
  TCR-CDR3 peptide sequence
CASSRDTYAEQFF

SEQ ID NO: 1391 - protein, 15 aa, Artificial sequence
  TCR-CDR3 peptide sequence
CASSRDWGNQDTQYF

SEQ ID NO: 1392 - protein, 14 aa, Artificial sequence
  TCR-CDR3 peptide sequence
CASSRGQNYAEQFF

SEQ ID NO: 1393 - protein, 14 aa, Artificial sequence
  TCR-CDR3 peptide sequence
CASSRLGDQDTQYF

SEQ ID NO: 1394 - protein, 14 aa, Artificial sequence
  TCR-CDR3 peptide sequence
CASSRLGDYAEQFF

SEQ ID NO: 1395 - protein, 13 aa, Artificial sequence
  TCR-CDR3 peptide sequence
CASSRQDQDTQYF

SEQ ID NO: 1396 - protein, 14 aa, Artificial sequence
  TCR-CDR3 peptide sequence
CASSRQGDTGQLYF

SEQ ID NO: 1397 - protein, 14 aa, Artificial sequence
  TCR-CDR3 peptide sequence
CASSRQGSAETLYF

SEQ ID NO: 1398 - protein, 14 aa, Artificial sequence
  TCR-CDR3 peptide sequence
CASSRQGSGNTLYF

SEQ ID NO: 1399 - protein, 12 aa, Artificial sequence
  TCR-CDR3 peptide sequence
CASSRQGTEVFF

SEQ ID NO: 1400 - protein, 14 aa, Artificial sequence
  TCR-CDR3 peptide sequence
CASSRQISNERLFF

SEQ ID NO: 1401 - protein, 11 aa, Artificial sequence
  TCR-CDR3 peptide sequence
CASSRQNTLYF

SEQ ID NO: 1402 - protein, 13 aa, Artificial sequence
  TCR-CDR3 peptide sequence
CASSRQSNTEVFF

SEQ ID NO: 1403 - protein, 13 aa, Artificial sequence
  TCR-CDR3 peptide sequence
CASSRQTNTEVFF

SEQ ID NO: 1404 - protein, 12 aa, Artificial sequence
  TCR-CDR3 peptide sequence
CASSRSAETLYF

SEQ ID NO: 1405 - protein, 13 aa, Artificial sequence
  TCR-CDR3 peptide sequence
CASSRTANTEVFF

SEQ ID NO: 1406 - protein, 12 aa, Artificial sequence
  TCR-CDR3 peptide sequence
CASSRTENTLYF

SEQ ID NO: 1407 - protein, 13 aa, Artificial sequence
  TCR-CDR3 peptide sequence
CASSRTGAETLYF

SEQ ID NO: 1408 - protein, 15 aa, Artificial sequence
  TCR-CDR3 peptide sequence
CASSRTGGNYAEQFF

SEQ ID NO: 1409 - protein, 15 aa, Artificial sequence
  TCR-CDR3 peptide sequence
CASSRTGGSQNTLYF

SEQ ID NO: 1410 - protein, 14 aa, Artificial sequence
  TCR-CDR3 peptide sequence
CASSRTGNYAEQFF

SEQ ID NO: 1411 - protein, 13 aa, Artificial sequence
  TCR-CDR3 peptide sequence
CASSRTSAETLYF

SEQ ID NO: 1412 - protein, 11 aa, Artificial sequence
  TCR-CDR3 peptide sequence
CASSSDAEQFF

SEQ ID NO: 1413 - protein, 14 aa, Artificial sequence
  TCR-CDR3 peptide sequence
CASSSGQNTGQLYF

SEQ ID NO: 1414 - protein, 14 aa, Artificial sequence
  TCR-CDR3 peptide sequence
CASSSGTGNERLFF

SEQ ID NO: 1415 - protein, 13 aa, Artificial sequence
  TCR-CDR3 peptide sequence
CASSSNSGNTLYF

SEQ ID NO: 1416 - protein, 11 aa, Artificial sequence
  TCR-CDR3 peptide sequence
CASSSNTEVFF

SEQ ID NO: 1417 - protein, 14 aa, Artificial sequence
  TCR-CDR3 peptide sequence
CASSSQGSGNTLYF

SEQ ID NO: 1418 - protein, 14 aa, Artificial sequence
  TCR-CDR3 peptide sequence
CASSSQGSQNTLYF

SEQ ID NO: 1419 - protein, 12 aa, Artificial sequence
  TCR-CDR3 peptide sequence
CASSSQGTEVFF

SEQ ID NO: 1420 - protein, 13 aa, Artificial sequence
  TCR-CDR3 peptide sequence
CASSSSSAETLYF

SEQ ID NO: 1421 - protein, 13 aa, Artificial sequence
  TCR-CDR3 peptide sequence
CASSSSSQNTLYF

SEQ ID NO: 1422 - protein, 14 aa, Artificial sequence
  TCR-CDR3 peptide sequence
CASSSTANTGQLYF

SEQ ID NO: 1423 - protein, 14 aa, Artificial sequence
  TCR-CDR3 peptide sequence
CASSSTGNTGQLYF

SEQ ID NO: 1424 - protein, 13 aa, Artificial sequence
  TCR-CDR3 peptide sequence
CASSSTSQNTLYF

SEQ ID NO: 1425 - protein, 13 aa, Artificial sequence
  TCR-CDR3 peptide sequence
CASSTGDQDTQYF

SEQ ID NO: 1426 - protein, 13 aa, Artificial sequence
  TCR-CDR3 peptide sequence
CASSTGGAETLYF

SEQ ID NO: 1427 - protein, 13 aa, Artificial sequence
  TCR-CDR3 peptide sequence
CASSTGGQNTLYF

SEQ ID NO: 1428 - protein, 13 aa, Artificial sequence
  TCR-CDR3 peptide sequence
CASSTGGYAEQFF

SEQ ID NO: 1429 - protein, 12 aa, Artificial sequence
  TCR-CDR3 peptide sequence
CASSTGNTEVFF

SEQ ID NO: 1430 - protein, 13 aa, Artificial sequence
  TCR-CDR3 peptide sequence
CASSTGSQNTLYF

SEQ ID NO: 1431 - protein, 12 aa, Artificial sequence
  TCR-CDR3 peptide sequence
CASSTNTGQLYF

SEQ ID NO: 1432 - protein, 13 aa, Artificial sequence
  TCR-CDR3 peptide sequence
CASSWDRNTEVFF

SEQ ID NO: 1433 - protein, 14 aa, Artificial sequence
  TCR-CDR3 peptide sequence
CGARDHTSNTEVFF

SEQ ID NO: 1434 - protein, 14 aa, Artificial sequence
  TCR-CDR3 peptide sequence
CGARDWGNTGQLYF

SEQ ID NO: 1435 - protein, 14 aa, Artificial sequence
  TCR-CDR3 peptide sequence
CGARDWGSQNTLYF

SEQ ID NO: 1436 - protein, 11 aa, Artificial sequence
  TCR-CDR3 peptide sequence
CSADSQNTLYF

SEQ ID NO: 1437 - protein, 14 aa, Artificial sequence
  TCR-CDR3 peptide sequence
CASGDAGANTEVFF

SEQ ID NO: 1438 - protein, 14 aa, Artificial sequence
  TCR-CDR3 peptide sequence
CASGDAGGNTEVFF

SEQ ID NO: 1439 - protein, 15 aa, Artificial sequence
  TCR-CDR3 peptide sequence
CASGDAGGNTGQLYF

SEQ ID NO: 1440 - protein, 15 aa, Artificial sequence
  TCR-CDR3 peptide sequence
CASGDAGGSQNTLYF

SEQ ID NO: 1441 - protein, 14 aa, Artificial sequence
  TCR-CDR3 peptide sequence
CASGDAGSGNTLYF

SEQ ID NO: 1442 - protein, 15 aa, Artificial sequence
  TCR-CDR3 peptide sequence
CASGDAGTANTEVFF

SEQ ID NO: 1443 - protein, 14 aa, Artificial sequence
  TCR-CDR3 peptide sequence
CASGDAGVQDTQYF

SEQ ID NO: 1444 - protein, 12 aa, Artificial sequence
  TCR-CDR3 peptide sequence
CASGDAQDTQYF

SEQ ID NO: 1445 - protein, 14 aa, Artificial sequence
  TCR-CDR3 peptide sequence
CASGDAQSQNTLYF

SEQ ID NO: 1446 - protein, 14 aa, Artificial sequence
  TCR-CDR3 peptide sequence
CASGDASSGNTLYF

SEQ ID NO: 1447 - protein, 15 aa, Artificial sequence
  TCR-CDR3 peptide sequence
CASGDATTSAETLYF

SEQ ID NO: 1448 - protein, 13 aa, Artificial sequence
  TCR-CDR3 peptide sequence
CASGDAYNSPLYF

SEQ ID NO: 1449 - protein, 14 aa, Artificial sequence
  TCR-CDR3 peptide sequence
CASGDGGNQDTQYF

SEQ ID NO: 1450 - protein, 12 aa, Artificial sequence
  TCR-CDR3 peptide sequence
CASGDGNTEVFF

SEQ ID NO: 1451 - protein, 14 aa, Artificial sequence
  TCR-CDR3 peptide sequence
CASGDGTTNTEVFF

SEQ ID NO: 1452 - protein, 11 aa, Artificial sequence
  TCR-CDR3 peptide sequence
CASGDNQAPLF

SEQ ID NO: 1453 - protein, 12 aa, Artificial sequence
  TCR-CDR3 peptide sequence
CASGDNQDTQYF

SEQ ID NO: 1454 - protein, 13 aa, Artificial sequence
  TCR-CDR3 peptide sequence
CASGDNSAETLYF

SEQ ID NO: 1455 - protein, 11 aa, Artificial sequence
  TCR-CDR3 peptide sequence
CASGDQNTLYF

SEQ ID NO: 1456 - protein, 13 aa, Artificial sequence
  TCR-CDR3 peptide sequence
CASGDRANSDYTF

SEQ ID NO: 1457 - protein, 12 aa, Artificial sequence
  TCR-CDR3 peptide sequence
CASGDRDTEVFF

SEQ ID NO: 1458 - protein, 14 aa, Artificial sequence
  TCR-CDR3 peptide sequence
CASGDRGNYAEQFF

SEQ ID NO: 1459 - protein, 12 aa, Artificial sequence
  TCR-CDR3 peptide sequence
CASGDRNSDYTF

SEQ ID NO: 1460 - protein, 13 aa, Artificial sequence
  TCR-CDR3 peptide sequence
CASGDSYNSPLYF

SEQ ID NO: 1461 - protein, 14 aa, Artificial sequence
  TCR-CDR3 peptide sequence
CASGDVGSQNTLYF

SEQ ID NO: 1462 - protein, 13 aa, Artificial sequence
  TCR-CDR3 peptide sequence
CASGDWGAETLYF

SEQ ID NO: 1463 - protein, 14 aa, Artificial sequence
  TCR-CDR3 peptide sequence
CASGDWGGYAEQFF

SEQ ID NO: 1464 - protein, 13 aa, Artificial sequence
  TCR-CDR3 peptide sequence
CASGDWGQDTQYF

SEQ ID NO: 1465 - protein, 11 aa, Artificial sequence
  TCR-CDR3 peptide sequence
CASGEQDTQYF

SEQ ID NO: 1466 - protein, 12 aa, Artificial sequence
  TCR-CDR3 peptide sequence
CASGESQNTLYF

SEQ ID NO: 1467 - protein, 13 aa, Artificial sequence
  TCR-CDR3 peptide sequence
CASGETANTEVFF

SEQ ID NO: 1468 - protein, 11 aa, Artificial sequence
  TCR-CDR3 peptide sequence
CASGNQDTQYF

SEQ ID NO: 1469 - protein, 13 aa, Artificial sequence
  TCR-CDR3 peptide sequence
CASGTGNYAEQFF

SEQ ID NO: 1470 - protein, 12 aa, Artificial sequence
  TCR-CDR3 peptide sequence
CASRDSGNTLYF

SEQ ID NO: 1471 - protein, 13 aa, Artificial sequence
  TCR-CDR3 peptide sequence
CASRDSSAETLYF

SEQ ID NO: 1472 - protein, 11 aa, Artificial sequence
  TCR-CDR3 peptide sequence
CASRSAETLYF

SEQ ID NO: 1473 - protein, 13 aa, Artificial sequence
  TCR-CDR3 peptide sequence
CASSAGSQNTLYF

SEQ ID NO: 1474 - protein, 12 aa, Artificial sequence
  TCR-CDR3 peptide sequence
CASSANTGQLYF

SEQ ID NO: 1475 - protein, 11 aa, Artificial sequence
  TCR-CDR3 peptide sequence
CASSDAETLYF

SEQ ID NO: 1476 - protein, 13 aa, Artificial sequence
  TCR-CDR3 peptide sequence
CASSDAGAETLYF

SEQ ID NO: 1477 - protein, 13 aa, Artificial sequence
  TCR-CDR3 peptide sequence
CASSDAGNTEVFF

SEQ ID NO: 1478 - protein, 14 aa, Artificial sequence
  TCR-CDR3 peptide sequence
CASSDASSGNTLYF

SEQ ID NO: 1479 - protein, 13 aa, Artificial sequence
  TCR-CDR3 peptide sequence
CASSDGSAETLYF

SEQ ID NO: 1480 - protein, 11 aa, Artificial sequence
  TCR-CDR3 peptide sequence
CASSDNERLFF

SEQ ID NO: 1481 - protein, 11 aa, Artificial sequence
  TCR-CDR3 peptide sequence
CASSDNSDYTF

SEQ ID NO: 1482 - protein, 12 aa, Artificial sequence
  TCR-CDR3 peptide sequence
CASSDNTGQLYF

SEQ ID NO: 1483 - protein, 13 aa, Artificial sequence
  TCR-CDR3 peptide sequence
CASSDRANTEVFF

SEQ ID NO: 1484 - protein, 14 aa, Artificial sequence
  TCR-CDR3 peptide sequence
CASSDRDSGNTLYF

SEQ ID NO: 1485 - protein, 12 aa, Artificial sequence
  TCR-CDR3 peptide sequence
CASSDRNERLFF

SEQ ID NO: 1486 - protein, 13 aa, Artificial sequence
  TCR-CDR3 peptide sequence
CASSDRSQNTLYF

SEQ ID NO: 1487 - protein, 14 aa, Artificial sequence
  TCR-CDR3 peptide sequence
CASSDSSYNSPLYF

SEQ ID NO: 1488 - protein, 11 aa, Artificial sequence
  TCR-CDR3 peptide sequence
CASSDTGQLYF

SEQ ID NO: 1489 - protein, 13 aa, Artificial sequence
  TCR-CDR3 peptide sequence
CASSDWGQDTQYF

SEQ ID NO: 1490 - protein, 14 aa, Artificial sequence
  TCR-CDR3 peptide sequence
CASSDWGSQNTLYF

SEQ ID NO: 1491 - protein, 13 aa, Artificial sequence
  TCR-CDR3 peptide sequence
CASSEQGNTEVFF

SEQ ID NO: 1492 - protein, 12 aa, Artificial sequence
  TCR-CDR3 peptide sequence
CASSESAETLYF

SEQ ID NO: 1493 - protein, 12 aa, Artificial sequence
  TCR-CDR3 peptide sequence
CASSFDTGQLYF

SEQ ID NO: 1494 - protein, 13 aa, Artificial sequence
  TCR-CDR3 peptide sequence
CASSFGQNTEVFF

SEQ ID NO: 1495 - protein, 13 aa, Artificial sequence
  TCR-CDR3 peptide sequence
CASSFNSAETLYF

SEQ ID NO: 1496 - protein, 12 aa, Artificial sequence
  TCR-CDR3 peptide sequence
CASSFQNTEVFF

SEQ ID NO: 1497 - protein, 12 aa, Artificial sequence
  TCR-CDR3 peptide sequence
CASSFSNERLFF

SEQ ID NO: 1498 - protein, 14 aa, Artificial sequence
  TCR-CDR3 peptide sequence
CASSFTGGQNTLYF

SEQ ID NO: 1499 - protein, 14 aa, Artificial sequence
  TCR-CDR3 peptide sequence
CASSFWGNYAEQFF

SEQ ID NO: 1500 - protein, 15 aa, Artificial sequence
  TCR-CDR3 peptide sequence
CASSGDWGNYAEQFF

SEQ ID NO: 1501 - protein, 12 aa, Artificial sequence
  TCR-CDR3 peptide sequence
CASSGQNTEVFF

SEQ ID NO: 1502 - protein, 14 aa, Artificial sequence
  TCR-CDR3 peptide sequence
CASSGTGGQDTQYF

SEQ ID NO: 1503 - protein, 12 aa, Artificial sequence
  TCR-CDR3 peptide sequence
CASSGTNTEVFF

SEQ ID NO: 1504 - protein, 12 aa, Artificial sequence
  TCR-CDR3 peptide sequence
CASSHNQDTQYF

SEQ ID NO: 1505 - protein, 13 aa, Artificial sequence
  TCR-CDR3 peptide sequence
CASSINSAETLYF

SEQ ID NO: 1506 - protein, 13 aa, Artificial sequence
  TCR-CDR3 peptide sequence
CASSIRGNTEVFF

SEQ ID NO: 1507 - protein, 14 aa, Artificial sequence
  TCR-CDR3 peptide sequence
CASSLAGGQNTLYF

SEQ ID NO: 1508 - protein, 13 aa, Artificial sequence
  TCR-CDR3 peptide sequence
CASSLAGNSDYTF

SEQ ID NO: 1509 - protein, 14 aa, Artificial sequence
  TCR-CDR3 peptide sequence
CASSLDGNQDTQYF

SEQ ID NO: 1510 - protein, 14 aa, Artificial sequence
  TCR-CDR3 peptide sequence
CASSLDGNYAEQFF

SEQ ID NO: 1511 - protein, 12 aa, Artificial sequence
  TCR-CDR3 peptide sequence
CASSLDNQAPLF

SEQ ID NO: 1512 - protein, 14 aa, Artificial sequence
  TCR-CDR3 peptide sequence
CASSLDSANTEVFF

SEQ ID NO: 1513 - protein, 12 aa, Artificial sequence
  TCR-CDR3 peptide sequence
CASSLDSTEVFF

SEQ ID NO: 1514 - protein, 14 aa, Artificial sequence
  TCR-CDR3 peptide sequence
CASSLDWGDAEQFF

SEQ ID NO: 1515 - protein, 12 aa, Artificial sequence
  TCR-CDR3 peptide sequence
CASSLEAETLYF

SEQ ID NO: 1516 - protein, 15 aa, Artificial sequence
  TCR-CDR3 peptide sequence
CASSLEGGNYAEQFF

SEQ ID NO: 1517 - protein, 13 aa, Artificial sequence
  TCR-CDR3 peptide sequence
CASSLEQNTEVFF

SEQ ID NO: 1518 - protein, 13 aa, Artificial sequence
  TCR-CDR3 peptide sequence
CASSLGAQNTLYF

SEQ ID NO: 1519 - protein, 14 aa, Artificial sequence
  TCR-CDR3 peptide sequence
CASSLGASAETLYF

SEQ ID NO: 1520 - protein, 14 aa, Artificial sequence
  TCR-CDR3 peptide sequence
CASSLGDNQDTQYF

SEQ ID NO: 1521 - protein, 11 aa, Artificial sequence
  TCR-CDR3 peptide sequence
CASSLGDTLYF

SEQ ID NO: 1522 - protein, 14 aa, Artificial sequence
  TCR-CDR3 peptide sequence
CASSLGGAGNTLYF

SEQ ID NO: 1523 - protein, 14 aa, Artificial sequence
  TCR-CDR3 peptide sequence
CASSLGGGAETLYF

SEQ ID NO: 1524 - protein, 13 aa, Artificial sequence
  TCR-CDR3 peptide sequence
CASSLGHYAEQFF

SEQ ID NO: 1525 - protein, 14 aa, Artificial sequence
  TCR-CDR3 peptide sequence
CASSLGLGENTLYF

SEQ ID NO: 1526 - protein, 14 aa, Artificial sequence
  TCR-CDR3 peptide sequence
CASSLGLSAETLYF

SEQ ID NO: 1527 - protein, 12 aa, Artificial sequence
  TCR-CDR3 peptide sequence
CASSLGNERLFF

SEQ ID NO: 1528 - protein, 15 aa, Artificial sequence
  TCR-CDR3 peptide sequence
CASSLGQANTGQLYF

SEQ ID NO: 1529 - protein, 15 aa, Artificial sequence
  TCR-CDR3 peptide sequence
CASSLGQGAGNTLYF

SEQ ID NO: 1530 - protein, 15 aa, Artificial sequence
  TCR-CDR3 peptide sequence
CASSLGQGNTGQLYF

SEQ ID NO: 1531 - protein, 15 aa, Artificial sequence
  TCR-CDR3 peptide sequence
CASSLGQGNYAEQFF

SEQ ID NO: 1532 - protein, 14 aa, Artificial sequence
  TCR-CDR3 peptide sequence
CASSLGQGQNTLYF

SEQ ID NO: 1533 - protein, 13 aa, Artificial sequence
  TCR-CDR3 peptide sequence
CASSLGQNQAPLF

SEQ ID NO: 1534 - protein, 14 aa, Artificial sequence
  TCR-CDR3 peptide sequence
CASSLGQYNSPLYF

SEQ ID NO: 1535 - protein, 13 aa, Artificial sequence
  TCR-CDR3 peptide sequence
CASSLGRNTEVFF

SEQ ID NO: 1536 - protein, 14 aa, Artificial sequence
  TCR-CDR3 peptide sequence
CASSLGSSGNTLYF

SEQ ID NO: 1537 - protein, 15 aa, Artificial sequence
  TCR-CDR3 peptide sequence
CASSLGTGGAETLYF

SEQ ID NO: 1538 - protein, 14 aa, Artificial sequence
  TCR-CDR3 peptide sequence
CASSLGTGNTEVFF

SEQ ID NO: 1539 - protein, 14 aa, Artificial sequence
  TCR-CDR3 peptide sequence
CASSLGTTNERLFF

SEQ ID NO: 1540 - protein, 14 aa, Artificial sequence
  TCR-CDR3 peptide sequence
CASSLLGNQDTQYF

SEQ ID NO: 1541 - protein, 14 aa, Artificial sequence
  TCR-CDR3 peptide sequence
CASSLLGNTGQLYF

SEQ ID NO: 1542 - protein, 14 aa, Artificial sequence
  TCR-CDR3 peptide sequence
CASSLLGSAETLYF

SEQ ID NO: 1543 - protein, 15 aa, Artificial sequence
  TCR-CDR3 peptide sequence
CASSLLGTSAETLYF

SEQ ID NO: 1544 - protein, 13 aa, Artificial sequence
  TCR-CDR3 peptide sequence
CASSLNNYAEQFF

SEQ ID NO: 1545 - protein, 12 aa, Artificial sequence
  TCR-CDR3 peptide sequence
CASSLNYAEQFF

SEQ ID NO: 1546 - protein, 12 aa, Artificial sequence
  TCR-CDR3 peptide sequence
CASSLQENTLYF

SEQ ID NO: 1547 - protein, 14 aa, Artificial sequence
  TCR-CDR3 peptide sequence
CASSLQGANERLFF

SEQ ID NO: 1548 - protein, 14 aa, Artificial sequence
  TCR-CDR3 peptide sequence
CASSLQGDTGQLYF

SEQ ID NO: 1549 - protein, 14 aa, Artificial sequence
  TCR-CDR3 peptide sequence
CASSLQGGAETLYF

SEQ ID NO: 1550 - protein, 14 aa, Artificial sequence
  TCR-CDR3 peptide sequence
CASSLQGGQNTLYF

SEQ ID NO: 1551 - protein, 13 aa, Artificial sequence
  TCR-CDR3 peptide sequence
CASSLQGGTEVFF

SEQ ID NO: 1552 - protein, 14 aa, Artificial sequence
  TCR-CDR3 peptide sequence
CASSLQGGYAEQFF

SEQ ID NO: 1553 - protein, 13 aa, Artificial sequence
  TCR-CDR3 peptide sequence
CASSLQGNQAPLF

SEQ ID NO: 1554 - protein, 14 aa, Artificial sequence
  TCR-CDR3 peptide sequence
CASSLQGTNERLFF

SEQ ID NO: 1555 - protein, 13 aa, Artificial sequence
  TCR-CDR3 peptide sequence
CASSLRANTEVFF

SEQ ID NO: 1556 - protein, 14 aa, Artificial sequence
  TCR-CDR3 peptide sequence
CASSLRDNYAEQFF

SEQ ID NO: 1557 - protein, 14 aa, Artificial sequence
  TCR-CDR3 peptide sequence
CASSLRDSGNTLYF

SEQ ID NO: 1558 - protein, 14 aa, Artificial sequence
  TCR-CDR3 peptide sequence
CASSLRGGQNTLYF

SEQ ID NO: 1559 - protein, 13 aa, Artificial sequence
  TCR-CDR3 peptide sequence
CASSLRGNSDYTF

SEQ ID NO: 1560 - protein, 13 aa, Artificial sequence
  TCR-CDR3 peptide sequence
CASSLSANSDYTF

SEQ ID NO: 1561 - protein, 13 aa, Artificial sequence
  TCR-CDR3 peptide sequence
CASSLSANTEVFF

SEQ ID NO: 1562 - protein, 12 aa, Artificial sequence
  TCR-CDR3 peptide sequence
CASSLSGTEVFF

SEQ ID NO: 1563 - protein, 14 aa, Artificial sequence
  TCR-CDR3 peptide sequence
CASSLSNSGNTLYF

SEQ ID NO: 1564 - protein, 14 aa, Artificial sequence
  TCR-CDR3 peptide sequence
CASSLTGANSDYTF

SEQ ID NO: 1565 - protein, 13 aa, Artificial sequence
  TCR-CDR3 peptide sequence
CASSLTGDSDYTF

SEQ ID NO: 1566 - protein, 13 aa, Artificial sequence
  TCR-CDR3 peptide sequence
CASSLTGDTEVFF

SEQ ID NO: 1567 - protein, 14 aa, Artificial sequence
  TCR-CDR3 peptide sequence
CASSLTGDTGQLYF

SEQ ID NO: 1568 - protein, 15 aa, Artificial sequence
  TCR-CDR3 peptide sequence
CASSLTGGNYAEQFF

SEQ ID NO: 1569 - protein, 14 aa, Artificial sequence
  TCR-CDR3 peptide sequence
CASSLTGNQDTQYF

SEQ ID NO: 1570 - protein, 13 aa, Artificial sequence
  TCR-CDR3 peptide sequence
CASSLTGYAEQFF

SEQ ID NO: 1571 - protein, 14 aa, Artificial sequence
  TCR-CDR3 peptide sequence
CASSLVGNTGQLYF

SEQ ID NO: 1572 - protein, 13 aa, Artificial sequence
  TCR-CDR3 peptide sequence
CASSLVGQNTLYF

SEQ ID NO: 1573 - protein, 12 aa, Artificial sequence
  TCR-CDR3 peptide sequence
CASSLVTGQLYF

SEQ ID NO: 1574 - protein, 11 aa, Artificial sequence
  TCR-CDR3 peptide sequence
CASSPDTEVFF

SEQ ID NO: 1575 - protein, 14 aa, Artificial sequence
  TCR-CDR3 peptide sequence
CASSPGANTGQLYF

SEQ ID NO: 1576 - protein, 14 aa, Artificial sequence
  TCR-CDR3 peptide sequence
CASSPGGSAETLYF

SEQ ID NO: 1577 - protein, 13 aa, Artificial sequence
  TCR-CDR3 peptide sequence
CASSPGGYAEQFF

SEQ ID NO: 1578 - protein, 14 aa, Artificial sequence
  TCR-CDR3 peptide sequence
CASSPGLGQNTLYF

SEQ ID NO: 1579 - protein, 15 aa, Artificial sequence
  TCR-CDR3 peptide sequence
CASSPGLGSQNTLYF

SEQ ID NO: 1580 - protein, 13 aa, Artificial sequence
  TCR-CDR3 peptide sequence
CASSPGNQDTQYF

SEQ ID NO: 1581 - protein, 13 aa, Artificial sequence
  TCR-CDR3 peptide sequence
CASSPGNTGQLYF

SEQ ID NO: 1582 - protein, 14 aa, Artificial sequence
  TCR-CDR3 peptide sequence
CASSPGQGNERLFF

SEQ ID NO: 1583 - protein, 13 aa, Artificial sequence
  TCR-CDR3 peptide sequence
CASSPGQKNTLYF

SEQ ID NO: 1584 - protein, 13 aa, Artificial sequence
  TCR-CDR3 peptide sequence
CASSPGQNQAPLF

SEQ ID NO: 1585 - protein, 14 aa, Artificial sequence
  TCR-CDR3 peptide sequence
CASSPGTANERLFF

SEQ ID NO: 1586 - protein, 13 aa, Artificial sequence
  TCR-CDR3 peptide sequence
CASSPGTDTEVFF

SEQ ID NO: 1587 - protein, 13 aa, Artificial sequence
  TCR-CDR3 peptide sequence
CASSPNSAETLYF

SEQ ID NO: 1588 - protein, 13 aa, Artificial sequence
  TCR-CDR3 peptide sequence
CASSPNSQNTLYF

SEQ ID NO: 1589 - protein, 14 aa, Artificial sequence
  TCR-CDR3 peptide sequence
CASSPQGANTEVFF

SEQ ID NO: 1590 - protein, 13 aa, Artificial sequence
  TCR-CDR3 peptide sequence
CASSPQGNSDYTF

SEQ ID NO: 1591 - protein, 13 aa, Artificial sequence
  TCR-CDR3 peptide sequence
CASSPRGAETLYF

SEQ ID NO: 1592 - protein, 13 aa, Artificial sequence
  TCR-CDR3 peptide sequence
CASSPSSQNTLYF

SEQ ID NO: 1593 - protein, 14 aa, Artificial sequence
  TCR-CDR3 peptide sequence
CASSPTGGAETLYF

SEQ ID NO: 1594 - protein, 14 aa, Artificial sequence
  TCR-CDR3 peptide sequence
CASSPTGSAETLYF

SEQ ID NO: 1595 - protein, 14 aa, Artificial sequence
  TCR-CDR3 peptide sequence
CASSPTINQDTQYF

SEQ ID NO: 1596 - protein, 13 aa, Artificial sequence
  TCR-CDR3 peptide sequence
CASSPTTNTEVFF

SEQ ID NO: 1597 - protein, 14 aa, Artificial sequence
  TCR-CDR3 peptide sequence
CASSPTVNQDTQYF

SEQ ID NO: 1598 - protein, 12 aa, Artificial sequence
  TCR-CDR3 peptide sequence
CASSQANTEVFF

SEQ ID NO: 1599 - protein, 14 aa, Artificial sequence
  TCR-CDR3 peptide sequence
CASSQDGSQNTLYF

SEQ ID NO: 1600 - protein, 14 aa, Artificial sequence
  TCR-CDR3 peptide sequence
CASSQDNSGNTLYF

SEQ ID NO: 1601 - protein, 15 aa, Artificial sequence
  TCR-CDR3 peptide sequence
CASSQDRGSAETLYF

SEQ ID NO: 1602 - protein, 14 aa, Artificial sequence
  TCR-CDR3 peptide sequence
CASSQDSYNSPLYF

SEQ ID NO: 1603 - protein, 14 aa, Artificial sequence
  TCR-CDR3 peptide sequence
CASSQDWGQNTLYF

SEQ ID NO: 1604 - protein, 13 aa, Artificial sequence
  TCR-CDR3 peptide sequence
CASSQGANSDYTF

SEQ ID NO: 1605 - protein, 13 aa, Artificial sequence
  TCR-CDR3 peptide sequence
CASSQGANTEVFF

SEQ ID NO: 1606 - protein, 12 aa, Artificial sequence
  TCR-CDR3 peptide sequence
CASSQGNTEVFF

SEQ ID NO: 1607 - protein, 11 aa, Artificial sequence
  TCR-CDR3 peptide sequence
CASSQGNTLYF

SEQ ID NO: 1608 - protein, 14 aa, Artificial sequence
  TCR-CDR3 peptide sequence
CASSQGTANSDYTF

SEQ ID NO: 1609 - protein, 14 aa, Artificial sequence
  TCR-CDR3 peptide sequence
CASSQQGSAETLYF

SEQ ID NO: 1610 - protein, 12 aa, Artificial sequence
  TCR-CDR3 peptide sequence
CASSQQGTEVFF

SEQ ID NO: 1611 - protein, 13 aa, Artificial sequence
  TCR-CDR3 peptide sequence
CASSQTANSDYTF

SEQ ID NO: 1612 - protein, 14 aa, Artificial sequence
  TCR-CDR3 peptide sequence
CASSQTGGQNTLYF

SEQ ID NO: 1613 - protein, 14 aa, Artificial sequence
  TCR-CDR3 peptide sequence
CASSRDKNTGQLYF

SEQ ID NO: 1614 - protein, 13 aa, Artificial sequence
  TCR-CDR3 peptide sequence
CASSRDNNQAPLF

SEQ ID NO: 1615 - protein, 14 aa, Artificial sequence
  TCR-CDR3 peptide sequence
CASSRDNNQDTQYF

SEQ ID NO: 1616 - protein, 13 aa, Artificial sequence
  TCR-CDR3 peptide sequence
CASSRDRYAEQFF

SEQ ID NO: 1617 - protein, 14 aa, Artificial sequence
  TCR-CDR3 peptide sequence
CASSRDSSNERLFF

SEQ ID NO: 1618 - protein, 13 aa, Artificial sequence
  TCR-CDR3 peptide sequence
CASSRDTNTEVFF

SEQ ID NO: 1619 - protein, 13 aa, Artificial sequence
  TCR-CDR3 peptide sequence
CASSRDTQDTQYF

SEQ ID NO: 1620 - protein, 13 aa, Artificial sequence
  TCR-CDR3 peptide sequence
CASSRDWGDTQYF

SEQ ID NO: 1621 - protein, 14 aa, Artificial sequence
  TCR-CDR3 peptide sequence
CASSRDWGSYEQYF

SEQ ID NO: 1622 - protein, 14 aa, Artificial sequence
  TCR-CDR3 peptide sequence
CASSRDWNYAEQFF

SEQ ID NO: 1623 - protein, 14 aa, Artificial sequence
  TCR-CDR3 peptide sequence
CASSRDWSAETLYF

SEQ ID NO: 1624 - protein, 12 aa, Artificial sequence
  TCR-CDR3 peptide sequence
CASSRDYAEQFF

SEQ ID NO: 1625 - protein, 12 aa, Artificial sequence
  TCR-CDR3 peptide sequence
CASSRGNTEVFF

SEQ ID NO: 1626 - protein, 13 aa, Artificial sequence
  TCR-CDR3 peptide sequence
CASSRGQNTEVFF

SEQ ID NO: 1627 - protein, 15 aa, Artificial sequence
  TCR-CDR3 peptide sequence
CASSRLGANTGQLYF

SEQ ID NO: 1628 - protein, 13 aa, Artificial sequence
  TCR-CDR3 peptide sequence
CASSRLGENTLYF

SEQ ID NO: 1629 - protein, 15 aa, Artificial sequence
  TCR-CDR3 peptide sequence
CASSRLGSSAETLYF

SEQ ID NO: 1630 - protein, 14 aa, Artificial sequence
  TCR-CDR3 peptide sequence
CASSRQANTGQLYF

SEQ ID NO: 1631 - protein, 12 aa, Artificial sequence
  TCR-CDR3 peptide sequence
CASSRQGETLYF

SEQ ID NO: 1632 - protein, 11 aa, Artificial sequence
  TCR-CDR3 peptide sequence
CASSRQGEVFF

SEQ ID NO: 1633 - protein, 13 aa, Artificial sequence
  TCR-CDR3 peptide sequence
CASSRQGYAEQFF

SEQ ID NO: 1634 - protein, 12 aa, Artificial sequence
  TCR-CDR3 peptide sequence
CASSRQQNTLYF

SEQ ID NO: 1635 - protein, 13 aa, Artificial sequence
  TCR-CDR3 peptide sequence
CASSRQSAETLYF

SEQ ID NO: 1636 - protein, 12 aa, Artificial sequence
  TCR-CDR3 peptide sequence
CASSRQYAEQFF

SEQ ID NO: 1637 - protein, 14 aa, Artificial sequence
  TCR-CDR3 peptide sequence
CASSRTASQNTLYF

SEQ ID NO: 1638 - protein, 13 aa, Artificial sequence
  TCR-CDR3 peptide sequence
CASSRTGGNTLYF

SEQ ID NO: 1639 - protein, 13 aa, Artificial sequence
  TCR-CDR3 peptide sequence
CASSRTGNTEVFF

SEQ ID NO: 1640 - protein, 12 aa, Artificial sequence
  TCR-CDR3 peptide sequence
CASSRTGNTLYF

SEQ ID NO: 1641 - protein, 14 aa, Artificial sequence
  TCR-CDR3 peptide sequence
CASSRTISNERLFF

SEQ ID NO: 1642 - protein, 14 aa, Artificial sequence
  TCR-CDR3 peptide sequence
CASSRTTSAETLYF

SEQ ID NO: 1643 - protein, 12 aa, Artificial sequence
  TCR-CDR3 peptide sequence
CASSSANTEVFF

SEQ ID NO: 1644 - protein, 14 aa, Artificial sequence
  TCR-CDR3 peptide sequence
CASSSDSSQNTLYF

SEQ ID NO: 1645 - protein, 14 aa, Artificial sequence
  TCR-CDR3 peptide sequence
CASSSDWGQNTLYF

SEQ ID NO: 1646 - protein, 13 aa, Artificial sequence
  TCR-CDR3 peptide sequence
CASSSGANTEVFF

SEQ ID NO: 1647 - protein, 12 aa, Artificial sequence
  TCR-CDR3 peptide sequence
CASSSGGTEVFF

SEQ ID NO: 1648 - protein, 14 aa, Artificial sequence
  TCR-CDR3 peptide sequence
CASSSGQNYAEQFF

SEQ ID NO: 1649 - protein, 14 aa, Artificial sequence
  TCR-CDR3 peptide sequence
CASSSGTANSDYTF

SEQ ID NO: 1650 - protein, 14 aa, Artificial sequence
  TCR-CDR3 peptide sequence
CASSSGTTNSDYTF

SEQ ID NO: 1651 - protein, 14 aa, Artificial sequence
  TCR-CDR3 peptide sequence
CASSSQGAGNTLYF

SEQ ID NO: 1652 - protein, 13 aa, Artificial sequence
  TCR-CDR3 peptide sequence
CASSSQGYAEQFF

SEQ ID NO: 1653 - protein, 14 aa, Artificial sequence
  TCR-CDR3 peptide sequence
CASSSTGDTGQLYF

SEQ ID NO: 1654 - protein, 13 aa, Artificial sequence
  TCR-CDR3 peptide sequence
CASSSTGNSDYTF

SEQ ID NO: 1655 - protein, 14 aa, Artificial sequence
  TCR-CDR3 peptide sequence
CASSTGGNYAEQFF

SEQ ID NO: 1656 - protein, 14 aa, Artificial sequence
  TCR-CDR3 peptide sequence
CASSTGTANTEVFF

SEQ ID NO: 1657 - protein, 12 aa, Artificial sequence
  TCR-CDR3 peptide sequence
CASSVNQDTQYF

SEQ ID NO: 1658 - protein, 12 aa, Artificial sequence
  TCR-CDR3 peptide sequence
CASSVSAETLYF

SEQ ID NO: 1659 - protein, 13 aa, Artificial sequence
  TCR-CDR3 peptide sequence
CASSWGDQDTQYF

SEQ ID NO: 1660 - protein, 14 aa, Artificial sequence
  TCR-CDR3 peptide sequence
CASSWTDSGNTLYF

SEQ ID NO: 1661 - protein, 13 aa, Artificial sequence
  TCR-CDR3 peptide sequence
CAWSLGDQDTQYF

SEQ ID NO: 1662 - protein, 14 aa, Artificial sequence
  TCR-CDR3 peptide sequence
CAWSLGGNYAEQFF

SEQ ID NO: 1663 - protein, 13 aa, Artificial sequence
  TCR-CDR3 peptide sequence
CAWSLGGQDTQYF

SEQ ID NO: 1664 - protein, 13 aa, Artificial sequence
  TCR-CDR3 peptide sequence
CAWSLGSAETLYF

SEQ ID NO: 1665 - protein, 12 aa, Artificial sequence
  TCR-CDR3 peptide sequence
CAWSLSAETLYF

SEQ ID NO: 1666 - protein, 12 aa, Artificial sequence
  TCR-CDR3 peptide sequence
CGARDNYAEQFF

SEQ ID NO: 1667 - protein, 13 aa, Artificial sequence
  TCR-CDR3 peptide sequence
CGARDRNTGQLYF

SEQ ID NO: 1668 - protein, 12 aa, Artificial sequence
  TCR-CDR3 peptide sequence
CGARDSQNTLYF

SEQ ID NO: 1669 - protein, 14 aa, Artificial sequence
  TCR-CDR3 peptide sequence
CGARDWGSAETLYF

SEQ ID NO: 1670 - protein, 9 aa, Artificial sequence
  TCR-CDR3 peptide sequence
CSADTEVFF

SEQ ID NO: 1671 - protein, 13 aa, Artificial sequence
  TCR-CDR3 peptide sequence
CASGDAANSDYTF

SEQ ID NO: 1672 - protein, 13 aa, Artificial sequence
  TCR-CDR3 peptide sequence
CASGDAGEDTQYF

SEQ ID NO: 1673 - protein, 12 aa, Artificial sequence
  TCR-CDR3 peptide sequence
CASGDAGGEQYF

SEQ ID NO: 1674 - protein, 15 aa, Artificial sequence
  TCR-CDR3 peptide sequence
CASGDAGGNQDTQYF

SEQ ID NO: 1675 - protein, 15 aa, Artificial sequence
  TCR-CDR3 peptide sequence
CASGDAGISNERLFF

SEQ ID NO: 1676 - protein, 14 aa, Artificial sequence
  TCR-CDR3 peptide sequence
CASGDAGQNSDYTF

SEQ ID NO: 1677 - protein, 14 aa, Artificial sequence
  TCR-CDR3 peptide sequence
CASGDAGVSYEQYF

SEQ ID NO: 1678 - protein, 12 aa, Artificial sequence
  TCR-CDR3 peptide sequence
CASGDARYEQYF

SEQ ID NO: 1679 - protein, 14 aa, Artificial sequence
  TCR-CDR3 peptide sequence
CASGDAWGQDTQYF

SEQ ID NO: 1680 - protein, 14 aa, Artificial sequence
  TCR-CDR3 peptide sequence
CASGDDRGQNTLYF

SEQ ID NO: 1681 - protein, 13 aa, Artificial sequence
  TCR-CDR3 peptide sequence
CASGDGGNTEVFF

SEQ ID NO: 1682 - protein, 14 aa, Artificial sequence
  TCR-CDR3 peptide sequence
CASGDGGSQNTLYF

SEQ ID NO: 1683 - protein, 13 aa, Artificial sequence
  TCR-CDR3 peptide sequence
CASGDGSAETLYF

SEQ ID NO: 1684 - protein, 14 aa, Artificial sequence
  TCR-CDR3 peptide sequence
CASGDGTANSDYTF

SEQ ID NO: 1685 - protein, 14 aa, Artificial sequence
  TCR-CDR3 peptide sequence
CASGDLGNYAEQFF

SEQ ID NO: 1686 - protein, 12 aa, Artificial sequence
  TCR-CDR3 peptide sequence
CASGDNNERLFF

SEQ ID NO: 1687 - protein, 11 aa, Artificial sequence
  TCR-CDR3 peptide sequence
CASGDNSDYTF

SEQ ID NO: 1688 - protein, 13 aa, Artificial sequence
  TCR-CDR3 peptide sequence
CASGDPANSDYTF

SEQ ID NO: 1689 - protein, 14 aa, Artificial sequence
  TCR-CDR3 peptide sequence
CASGDRDNYAEQFF

SEQ ID NO: 1690 - protein, 14 aa, Artificial sequence
  TCR-CDR3 peptide sequence
CASGDRGNTGQLYF

SEQ ID NO: 1691 - protein, 13 aa, Artificial sequence
  TCR-CDR3 peptide sequence
CASGDRGQDTQYF

SEQ ID NO: 1692 - protein, 13 aa, Artificial sequence
  TCR-CDR3 peptide sequence
CASGDSANTEVFF

SEQ ID NO: 1693 - protein, 13 aa, Artificial sequence
  TCR-CDR3 peptide sequence
CASGDTSAETLYF

SEQ ID NO: 1694 - protein, 11 aa, Artificial sequence
  TCR-CDR3 peptide sequence
CASGEEDTQYF

SEQ ID NO: 1695 - protein, 13 aa, Artificial sequence
  TCR-CDR3 peptide sequence
CASGETGNTEVFF

SEQ ID NO: 1696 - protein, 14 aa, Artificial sequence
  TCR-CDR3 peptide sequence
CASGETGNYAEQFF

SEQ ID NO: 1697 - protein, 9 aa, Artificial sequence
  TCR-CDR3 peptide sequence
CASGETLYF

SEQ ID NO: 1698 - protein, 14 aa, Artificial sequence
  TCR-CDR3 peptide sequence
CASGPGQGYAEQFF

SEQ ID NO: 1699 - protein, 11 aa, Artificial sequence
  TCR-CDR3 peptide sequence
CASGSQNTLYF

SEQ ID NO: 1700 - protein, 12 aa, Artificial sequence
  TCR-CDR3 peptide sequence
CASGTANTEVFF

SEQ ID NO: 1701 - protein, 12 aa, Artificial sequence
  TCR-CDR3 peptide sequence
CASGTGNSDYTF

SEQ ID NO: 1702 - protein, 13 aa, Artificial sequence
  TCR-CDR3 peptide sequence
CASRDNANTEVFF

SEQ ID NO: 1703 - protein, 12 aa, Artificial sequence
  TCR-CDR3 peptide sequence
CASRDNQDTQYF

SEQ ID NO: 1704 - protein, 13 aa, Artificial sequence
  TCR-CDR3 peptide sequence
CASRGQGNTEVFF

SEQ ID NO: 1705 - protein, 13 aa, Artificial sequence
  TCR-CDR3 peptide sequence
CASRQGANTEVFF

SEQ ID NO: 1706 - protein, 11 aa, Artificial sequence
  TCR-CDR3 peptide sequence
CASRQNTEVFF

SEQ ID NO: 1707 - protein, 11 aa, Artificial sequence
  TCR-CDR3 peptide sequence
CASRSQNTLYF

SEQ ID NO: 1708 - protein, 13 aa, Artificial sequence
  TCR-CDR3 peptide sequence
CASSADSAETLYF

SEQ ID NO: 1709 - protein, 14 aa, Artificial sequence
  TCR-CDR3 peptide sequence
CASSAGTANTEVFF

SEQ ID NO: 1710 - protein, 14 aa, Artificial sequence
  TCR-CDR3 peptide sequence
CASSDAGSAETLYF

SEQ ID NO: 1711 - protein, 13 aa, Artificial sequence
  TCR-CDR3 peptide sequence
CASSDGGNTEVFF

SEQ ID NO: 1712 - protein, 14 aa, Artificial sequence
  TCR-CDR3 peptide sequence
CASSDGTANSDYTF

SEQ ID NO: 1713 - protein, 13 aa, Artificial sequence
  TCR-CDR3 peptide sequence
CASSDNSAETLYF

SEQ ID NO: 1714 - protein, 13 aa, Artificial sequence
  TCR-CDR3 peptide sequence
CASSDPGQDTQYF

SEQ ID NO: 1715 - protein, 13 aa, Artificial sequence
  TCR-CDR3 peptide sequence
CASSDRDQDTQYF

SEQ ID NO: 1716 - protein, 11 aa, Artificial sequence
  TCR-CDR3 peptide sequence
CASSDRETLYF

SEQ ID NO: 1717 - protein, 13 aa, Artificial sequence
  TCR-CDR3 peptide sequence
CASSDRGQDTQYF

SEQ ID NO: 1718 - protein, 14 aa, Artificial sequence
  TCR-CDR3 peptide sequence
CASSDRGSAETLYF

SEQ ID NO: 1719 - protein, 13 aa, Artificial sequence
  TCR-CDR3 peptide sequence
CASSDTANTEVFF

SEQ ID NO: 1720 - protein, 11 aa, Artificial sequence
  TCR-CDR3 peptide sequence
CASSEDTEVFF

SEQ ID NO: 1721 - protein, 13 aa, Artificial sequence
  TCR-CDR3 peptide sequence
CASSEGAGNTLYF

SEQ ID NO: 1722 - protein, 14 aa, Artificial sequence
  TCR-CDR3 peptide sequence
CASSFDNSGNTLYF

SEQ ID NO: 1723 - protein, 13 aa, Artificial sequence
  TCR-CDR3 peptide sequence
CASSFDRNTEVFF

SEQ ID NO: 1724 - protein, 11 aa, Artificial sequence
  TCR-CDR3 peptide sequence
CASSFGERLFF

SEQ ID NO: 1725 - protein, 14 aa, Artificial sequence
  TCR-CDR3 peptide sequence
CASSFLGNYAEQFF

SEQ ID NO: 1726 - protein, 11 aa, Artificial sequence
  TCR-CDR3 peptide sequence
CASSFNERLFF

SEQ ID NO: 1727 - protein, 13 aa, Artificial sequence
  TCR-CDR3 peptide sequence
CASSFQANSDYTF

SEQ ID NO: 1728 - protein, 13 aa, Artificial sequence
  TCR-CDR3 peptide sequence
CASSFQNYAEQFF

SEQ ID NO: 1729 - protein, 14 aa, Artificial sequence
  TCR-CDR3 peptide sequence
CASSFRTENTEVFF

SEQ ID NO: 1730 - protein, 13 aa, Artificial sequence
  TCR-CDR3 peptide sequence
CASSFSSGNTLYF

SEQ ID NO: 1731 - protein, 13 aa, Artificial sequence
  TCR-CDR3 peptide sequence
CASSFTANTEVFF

SEQ ID NO: 1732 - protein, 12 aa, Artificial sequence
  TCR-CDR3 peptide sequence
CASSGGAETLYF

SEQ ID NO: 1733 - protein, 10 aa, Artificial sequence
  TCR-CDR3 peptide sequence
CASSGNTLYF

SEQ ID NO: 1734 - protein, 13 aa, Artificial sequence
  TCR-CDR3 peptide sequence
CASSGQNTGQLYF

SEQ ID NO: 1735 - protein, 13 aa, Artificial sequence
  TCR-CDR3 peptide sequence
CASSGTDYAEQFF

SEQ ID NO: 1736 - protein, 13 aa, Artificial sequence
  TCR-CDR3 peptide sequence
CASSGTGGNTLYF

SEQ ID NO: 1737 - protein, 12 aa, Artificial sequence
  TCR-CDR3 peptide sequence
CASSGTGNTLYF

SEQ ID NO: 1738 - protein, 13 aa, Artificial sequence
  TCR-CDR3 peptide sequence
CASSGTSAETLYF

SEQ ID NO: 1739 - protein, 14 aa, Artificial sequence
  TCR-CDR3 peptide sequence
CASSGTSSAETLYF

SEQ ID NO: 1740 - protein, 14 aa, Artificial sequence
  TCR-CDR3 peptide sequence
CASSGTTSAETLYF

SEQ ID NO: 1741 - protein, 12 aa, Artificial sequence
  TCR-CDR3 peptide sequence
CASSHNYAEQFF

SEQ ID NO: 1742 - protein, 12 aa, Artificial sequence
  TCR-CDR3 peptide sequence
CASSIGAETLYF

SEQ ID NO: 1743 - protein, 14 aa, Artificial sequence
  TCR-CDR3 peptide sequence
CASSIGGNYAEQFF

SEQ ID NO: 1744 - protein, 14 aa, Artificial sequence
  TCR-CDR3 peptide sequence
CASSLAGGAETLYF

SEQ ID NO: 1745 - protein, 13 aa, Artificial sequence
  TCR-CDR3 peptide sequence
CASSLAQNTEVFF

SEQ ID NO: 1746 - protein, 12 aa, Artificial sequence
  TCR-CDR3 peptide sequence
CASSLAQNTLYF

SEQ ID NO: 1747 - protein, 14 aa, Artificial sequence
  TCR-CDR3 peptide sequence
CASSLASSGNTLYF

SEQ ID NO: 1748 - protein, 15 aa, Artificial sequence
  TCR-CDR3 peptide sequence
CASSLDGGSAETLYF

SEQ ID NO: 1749 - protein, 13 aa, Artificial sequence
  TCR-CDR3 peptide sequence
CASSLDGNSDYTF

SEQ ID NO: 1750 - protein, 14 aa, Artificial sequence
  TCR-CDR3 peptide sequence
CASSLDGNTGQLYF

SEQ ID NO: 1751 - protein, 14 aa, Artificial sequence
  TCR-CDR3 peptide sequence
CASSLDGSAETLYF

SEQ ID NO: 1752 - protein, 11 aa, Artificial sequence
  TCR-CDR3 peptide sequence
CASSLDKEVFF

SEQ ID NO: 1753 - protein, 13 aa, Artificial sequence
  TCR-CDR3 peptide sequence
CASSLDNNERLFF

SEQ ID NO: 1754 - protein, 13 aa, Artificial sequence
  TCR-CDR3 peptide sequence
CASSLDNNQAPLF

SEQ ID NO: 1755 - protein, 14 aa, Artificial sequence
  TCR-CDR3 peptide sequence
CASSLDRANTEVFF

SEQ ID NO: 1756 - protein, 14 aa, Artificial sequence
  TCR-CDR3 peptide sequence
CASSLDRGQNTLYF

SEQ ID NO: 1757 - protein, 14 aa, Artificial sequence
  TCR-CDR3 peptide sequence
CASSLDRGYAEQFF

SEQ ID NO: 1758 - protein, 14 aa, Artificial sequence
  TCR-CDR3 peptide sequence
CASSLDSNTGQLYF

SEQ ID NO: 1759 - protein, 14 aa, Artificial sequence
  TCR-CDR3 peptide sequence
CASSLDSSNERLFF

SEQ ID NO: 1760 - protein, 14 aa, Artificial sequence
  TCR-CDR3 peptide sequence
CASSLDWGAETLYF

SEQ ID NO: 1761 - protein, 11 aa, Artificial sequence
  TCR-CDR3 peptide sequence
CASSLEETLYF

SEQ ID NO: 1762 - protein, 14 aa, Artificial sequence
  TCR-CDR3 peptide sequence
CASSLEGAQDTQYF

SEQ ID NO: 1763 - protein, 15 aa, Artificial sequence
  TCR-CDR3 peptide sequence
CASSLEGASQNTLYF

SEQ ID NO: 1764 - protein, 14 aa, Artificial sequence
  TCR-CDR3 peptide sequence
CASSLEGGAETLYF

SEQ ID NO: 1765 - protein, 13 aa, Artificial sequence
  TCR-CDR3 peptide sequence
CASSLEGGNTLYF

SEQ ID NO: 1766 - protein, 15 aa, Artificial sequence
  TCR-CDR3 peptide sequence
CASSLEGNSGNTLYF

SEQ ID NO: 1767 - protein, 14 aa, Artificial sequence
  TCR-CDR3 peptide sequence
CASSLEGRQNTLYF

SEQ ID NO: 1768 - protein, 14 aa, Artificial sequence
  TCR-CDR3 peptide sequence
CASSLEGTGNTLYF

SEQ ID NO: 1769 - protein, 14 aa, Artificial sequence
  TCR-CDR3 peptide sequence
CASSLEQGAETLYF

SEQ ID NO: 1770 - protein, 14 aa, Artificial sequence
  TCR-CDR3 peptide sequence
CASSLEQNTGQLYF

SEQ ID NO: 1771 - protein, 13 aa, Artificial sequence
  TCR-CDR3 peptide sequence
CASSLEQYAEQFF

SEQ ID NO: 1772 - protein, 13 aa, Artificial sequence
  TCR-CDR3 peptide sequence
CASSLGGENTLYF

SEQ ID NO: 1773 - protein, 12 aa, Artificial sequence
  TCR-CDR3 peptide sequence
CASSLGGETLYF

SEQ ID NO: 1774 - protein, 14 aa, Artificial sequence
  TCR-CDR3 peptide sequence
CASSLGGRAETLYF

SEQ ID NO: 1775 - protein, 12 aa, Artificial sequence
  TCR-CDR3 peptide sequence
CASSLGHTEVFF

SEQ ID NO: 1776 - protein, 15 aa, Artificial sequence
  TCR-CDR3 peptide sequence
CASSLGLGNYAEQFF

SEQ ID NO: 1777 - protein, 14 aa, Artificial sequence
  TCR-CDR3 peptide sequence
CASSLGLGQNTLYF

SEQ ID NO: 1778 - protein, 14 aa, Artificial sequence
  TCR-CDR3 peptide sequence
CASSLGLNQDTQYF

SEQ ID NO: 1779 - protein, 14 aa, Artificial sequence
  TCR-CDR3 peptide sequence
CASSLGLNYAEQFF

SEQ ID NO: 1780 - protein, 14 aa, Artificial sequence
  TCR-CDR3 peptide sequence
CASSLGLSQNTLYF

SEQ ID NO: 1781 - protein, 13 aa, Artificial sequence
  TCR-CDR3 peptide sequence
CASSLGPNTEVFF

SEQ ID NO: 1782 - protein, 14 aa, Artificial sequence
  TCR-CDR3 peptide sequence
CASSLGQGNERLFF

SEQ ID NO: 1783 - protein, 13 aa, Artificial sequence
  TCR-CDR3 peptide sequence
CASSLGQNERLFF

SEQ ID NO: 1784 - protein, 13 aa, Artificial sequence
  TCR-CDR3 peptide sequence
CASSLGSQDTQYF

SEQ ID NO: 1785 - protein, 13 aa, Artificial sequence
  TCR-CDR3 peptide sequence
CASSLGTAETLYF

SEQ ID NO: 1786 - protein, 14 aa, Artificial sequence
  TCR-CDR3 peptide sequence
CASSLGTDYAEQFF

SEQ ID NO: 1787 - protein, 14 aa, Artificial sequence
  TCR-CDR3 peptide sequence
CASSLGTGYAEQFF

SEQ ID NO: 1788 - protein, 14 aa, Artificial sequence
  TCR-CDR3 peptide sequence
CASSLGTNQDTQYF

SEQ ID NO: 1789 - protein, 15 aa, Artificial sequence
  TCR-CDR3 peptide sequence
CASSLGTTSAETLYF

SEQ ID NO: 1790 - protein, 13 aa, Artificial sequence
  TCR-CDR3 peptide sequence
CASSLGTYAEQFF

SEQ ID NO: 1791 - protein, 13 aa, Artificial sequence
  TCR-CDR3 peptide sequence
CASSLLGAETLYF

SEQ ID NO: 1792 - protein, 13 aa, Artificial sequence
  TCR-CDR3 peptide sequence
CASSLLGENTLYF

SEQ ID NO: 1793 - protein, 14 aa, Artificial sequence
  TCR-CDR3 peptide sequence
CASSLLGGQNTLYF

SEQ ID NO: 1794 - protein, 12 aa, Artificial sequence
  TCR-CDR3 peptide sequence
CASSLNAETLYF

SEQ ID NO: 1795 - protein, 12 aa, Artificial sequence
  TCR-CDR3 peptide sequence
CASSLNNERLFF

SEQ ID NO: 1796 - protein, 10 aa, Artificial sequence
  TCR-CDR3 peptide sequence
CASSLNTLYF

SEQ ID NO: 1797 - protein, 14 aa, Artificial sequence
  TCR-CDR3 peptide sequence
CASSLPGSQNTLYF

SEQ ID NO: 1798 - protein, 12 aa, Artificial sequence
  TCR-CDR3 peptide sequence
CASSLQGERLFF

SEQ ID NO: 1799 - protein, 14 aa, Artificial sequence
  TCR-CDR3 peptide sequence
CASSLQNSGNTLYF

SEQ ID NO: 1800 - protein, 12 aa, Artificial sequence
  TCR-CDR3 peptide sequence
CASSLQYAEQFF

SEQ ID NO: 1801 - protein, 14 aa, Artificial sequence
  TCR-CDR3 peptide sequence
CASSLRANTGQLYF

SEQ ID NO: 1802 - protein, 11 aa, Artificial sequence
  TCR-CDR3 peptide sequence
CASSLRDTLYF

SEQ ID NO: 1803 - protein, 13 aa, Artificial sequence
  TCR-CDR3 peptide sequence
CASSLRGYAEQFF

SEQ ID NO: 1804 - protein, 14 aa, Artificial sequence
  TCR-CDR3 peptide sequence
CASSLRNSGNTLYF

SEQ ID NO: 1805 - protein, 12 aa, Artificial sequence
  TCR-CDR3 peptide sequence
CASSLRNTEVFF

SEQ ID NO: 1806 - protein, 12 aa, Artificial sequence
  TCR-CDR3 peptide sequence
CASSLRQNTLYF

SEQ ID NO: 1807 - protein, 13 aa, Artificial sequence
  TCR-CDR3 peptide sequence
CASSLSGNSDYTF

SEQ ID NO: 1808 - protein, 12 aa, Artificial sequence
  TCR-CDR3 peptide sequence
CASSLSNSDYTF

SEQ ID NO: 1809 - protein, 13 aa, Artificial sequence
  TCR-CDR3 peptide sequence
CASSLSTNSDYTF

SEQ ID NO: 1810 - protein, 14 aa, Artificial sequence
  TCR-CDR3 peptide sequence
CASSLSVNQDTQYF

SEQ ID NO: 1811 - protein, 14 aa, Artificial sequence
  TCR-CDR3 peptide sequence
CASSLTGSGNTLYF

SEQ ID NO: 1812 - protein, 12 aa, Artificial sequence
  TCR-CDR3 peptide sequence
CASSLTGTEVFF

SEQ ID NO: 1813 - protein, 13 aa, Artificial sequence
  TCR-CDR3 peptide sequence
CASSLTPNTEVFF

SEQ ID NO: 1814 - protein, 14 aa, Artificial sequence
  TCR-CDR3 peptide sequence
CASSLTTNTGQLYF

SEQ ID NO: 1815 - protein, 13 aa, Artificial sequence
  TCR-CDR3 peptide sequence
CASSLVANSDYTF

SEQ ID NO: 1816 - protein, 14 aa, Artificial sequence
  TCR-CDR3 peptide sequence
CASSLVGSAETLYF

SEQ ID NO: 1817 - protein, 14 aa, Artificial sequence
  TCR-CDR3 peptide sequence
CASSLVNSGNTLYF

SEQ ID NO: 1818 - protein, 12 aa, Artificial sequence
  TCR-CDR3 peptide sequence
CASSLVQNTLYF

SEQ ID NO: 1819 - protein, 13 aa, Artificial sequence
  TCR-CDR3 peptide sequence
CASSPANTGQLYF

SEQ ID NO: 1820 - protein, 14 aa, Artificial sequence
  TCR-CDR3 peptide sequence
CASSPDNSQNTLYF

SEQ ID NO: 1821 - protein, 14 aa, Artificial sequence
  TCR-CDR3 peptide sequence
CASSPDTSQNTLYF

SEQ ID NO: 1822 - protein, 12 aa, Artificial sequence
  TCR-CDR3 peptide sequence
CASSPGDTEVFF

SEQ ID NO: 1823 - protein, 13 aa, Artificial sequence
  TCR-CDR3 peptide sequence
CASSPGHQDTQYF

SEQ ID NO: 1824 - protein, 15 aa, Artificial sequence
  TCR-CDR3 peptide sequence
CASSPGLGSAETLYF

SEQ ID NO: 1825 - protein, 14 aa, Artificial sequence
  TCR-CDR3 peptide sequence
CASSPGLSQNTLYF

SEQ ID NO: 1826 - protein, 13 aa, Artificial sequence
  TCR-CDR3 peptide sequence
CASSPGQGNTLYF

SEQ ID NO: 1827 - protein, 15 aa, Artificial sequence
  TCR-CDR3 peptide sequence
CASSPGQISNERLFF

SEQ ID NO: 1828 - protein, 14 aa, Artificial sequence
  TCR-CDR3 peptide sequence
CASSPGQNNQAPLF

SEQ ID NO: 1829 - protein, 14 aa, Artificial sequence
  TCR-CDR3 peptide sequence
CASSPGTGNSDYTF

SEQ ID NO: 1830 - protein, 14 aa, Artificial sequence
  TCR-CDR3 peptide sequence
CASSPGTGNTEVFF

SEQ ID NO: 1831 - protein, 14 aa, Artificial sequence
  TCR-CDR3 peptide sequence
CASSPQGDTGQLYF

SEQ ID NO: 1832 - protein, 14 aa, Artificial sequence
  TCR-CDR3 peptide sequence
CASSPQGNYAEQFF

SEQ ID NO: 1833 - protein, 12 aa, Artificial sequence
  TCR-CDR3 peptide sequence
CASSPQNTEVFF

SEQ ID NO: 1834 - protein, 14 aa, Artificial sequence
  TCR-CDR3 peptide sequence
CASSPRDSAETLYF

SEQ ID NO: 1835 - protein, 14 aa, Artificial sequence
  TCR-CDR3 peptide sequence
CASSPRLGQDTQYF

SEQ ID NO: 1836 - protein, 12 aa, Artificial sequence
  TCR-CDR3 peptide sequence
CASSPSGNTLYF

SEQ ID NO: 1837 - protein, 13 aa, Artificial sequence
  TCR-CDR3 peptide sequence
CASSPTGNTEVFF

SEQ ID NO: 1838 - protein, 13 aa, Artificial sequence
  TCR-CDR3 peptide sequence
CASSPTGYAEQFF

SEQ ID NO: 1839 - protein, 14 aa, Artificial sequence
  TCR-CDR3 peptide sequence
CASSPTISNERLFF

SEQ ID NO: 1840 - protein, 14 aa, Artificial sequence
  TCR-CDR3 peptide sequence
CASSPTNSGNTLYF

SEQ ID NO: 1841 - protein, 12 aa, Artificial sequence
  TCR-CDR3 peptide sequence
CASSPTNTEVFF

SEQ ID NO: 1842 - protein, 14 aa, Artificial sequence
  TCR-CDR3 peptide sequence
CASSPWGNYAEQFF

SEQ ID NO: 1843 - protein, 14 aa, Artificial sequence
  TCR-CDR3 peptide sequence
CASSQDRANTEVFF

SEQ ID NO: 1844 - protein, 14 aa, Artificial sequence
  TCR-CDR3 peptide sequence
CASSQDSNQDTQYF

SEQ ID NO: 1845 - protein, 14 aa, Artificial sequence
  TCR-CDR3 peptide sequence
CASSQDWDQDTQYF

SEQ ID NO: 1846 - protein, 15 aa, Artificial sequence
  TCR-CDR3 peptide sequence
CASSQDWGSAETLYF

SEQ ID NO: 1847 - protein, 14 aa, Artificial sequence
  TCR-CDR3 peptide sequence
CASSQEGNQDTQYF

SEQ ID NO: 1848 - protein, 13 aa, Artificial sequence
  TCR-CDR3 peptide sequence
CASSQEGNTEVFF

SEQ ID NO: 1849 - protein, 14 aa, Artificial sequence
  TCR-CDR3 peptide sequence
CASSQEGQQDTQYF

SEQ ID NO: 1850 - protein, 14 aa, Artificial sequence
  TCR-CDR3 peptide sequence
CASSQGNSGNTLYF

SEQ ID NO: 1851 - protein, 13 aa, Artificial sequence
  TCR-CDR3 peptide sequence
CASSQGQQNTLYF

SEQ ID NO: 1852 - protein, 15 aa, Artificial sequence
  TCR-CDR3 peptide sequence
CASSQGTASAETLYF

SEQ ID NO: 1853 - protein, 15 aa, Artificial sequence
  TCR-CDR3 peptide sequence
CASSQGTISNERLFF

SEQ ID NO: 1854 - protein, 12 aa, Artificial sequence
  TCR-CDR3 peptide sequence
CASSQNYAEQFF

SEQ ID NO: 1855 - protein, 14 aa, Artificial sequence
  TCR-CDR3 peptide sequence
CASSQQGAGNTLYF

SEQ ID NO: 1856 - protein, 14 aa, Artificial sequence
  TCR-CDR3 peptide sequence
CASSQTGNTGQLYF

SEQ ID NO: 1857 - protein, 14 aa, Artificial sequence
  TCR-CDR3 peptide sequence
CASSRDINQDTQYF

SEQ ID NO: 1858 - protein, 14 aa, Artificial sequence
  TCR-CDR3 peptide sequence
CASSRDISGNTLYF

SEQ ID NO: 1859 - protein, 13 aa, Artificial sequence
  TCR-CDR3 peptide sequence
CASSRDKNTEVFF

SEQ ID NO: 1860 - protein, 14 aa, Artificial sequence
  TCR-CDR3 peptide sequence
CASSRDNANSDYTF

SEQ ID NO: 1861 - protein, 14 aa, Artificial sequence
  TCR-CDR3 peptide sequence
CASSRDRGNTEVFF

SEQ ID NO: 1862 - protein, 13 aa, Artificial sequence
  TCR-CDR3 peptide sequence
CASSRDRGTEVFF

SEQ ID NO: 1863 - protein, 14 aa, Artificial sequence
  TCR-CDR3 peptide sequence
CASSRDRSQNTLYF

SEQ ID NO: 1864 - protein, 13 aa, Artificial sequence
  TCR-CDR3 peptide sequence
CASSRDSNTEVFF

SEQ ID NO: 1865 - protein, 14 aa, Artificial sequence
  TCR-CDR3 peptide sequence
CASSRDTNYAEQFF

SEQ ID NO: 1866 - protein, 13 aa, Artificial sequence
  TCR-CDR3 peptide sequence
CASSRDWEDTQYF

SEQ ID NO: 1867 - protein, 14 aa, Artificial sequence
  TCR-CDR3 peptide sequence
CASSRDWGAETLYF

SEQ ID NO: 1868 - protein, 14 aa, Artificial sequence
  TCR-CDR3 peptide sequence
CASSRDWGQNTLYF

SEQ ID NO: 1869 - protein, 13 aa, Artificial sequence
  TCR-CDR3 peptide sequence
CASSRGDQDTQYF

SEQ ID NO: 1870 - protein, 13 aa, Artificial sequence
  TCR-CDR3 peptide sequence
CASSRGGQNTLYF

SEQ ID NO: 1871 - protein, 14 aa, Artificial sequence
  TCR-CDR3 peptide sequence
CASSRGQGYAEQFF

SEQ ID NO: 1872 - protein, 12 aa, Artificial sequence
  TCR-CDR3 peptide sequence
CASSRNYAEQFF

SEQ ID NO: 1873 - protein, 13 aa, Artificial sequence
  TCR-CDR3 peptide sequence
CASSRQGDSDYTF

SEQ ID NO: 1874 - protein, 12 aa, Artificial sequence
  TCR-CDR3 peptide sequence
CASSRQNQAPLF

SEQ ID NO: 1875 - protein, 12 aa, Artificial sequence
  TCR-CDR3 peptide sequence
CASSRTAETLYF

SEQ ID NO: 1876 - protein, 14 aa, Artificial sequence
  TCR-CDR3 peptide sequence
CASSRTGGNTEVFF

SEQ ID NO: 1877 - protein, 14 aa, Artificial sequence
  TCR-CDR3 peptide sequence
CASSSDSSGNTLYF

SEQ ID NO: 1878 - protein, 12 aa, Artificial sequence
  TCR-CDR3 peptide sequence
CASSSGENTLYF

SEQ ID NO: 1879 - protein, 14 aa, Artificial sequence
  TCR-CDR3 peptide sequence
CASSSGGNYAEQFF

SEQ ID NO: 1880 - protein, 14 aa, Artificial sequence
  TCR-CDR3 peptide sequence
CASSSGGSAETLYF

SEQ ID NO: 1881 - protein, 14 aa, Artificial sequence
  TCR-CDR3 peptide sequence
CASSSGGSQNTLYF

SEQ ID NO: 1882 - protein, 14 aa, Artificial sequence
  TCR-CDR3 peptide sequence
CASSSGLGQDTQYF

SEQ ID NO: 1883 - protein, 13 aa, Artificial sequence
  TCR-CDR3 peptide sequence
CASSSGNTGQLYF

SEQ ID NO: 1884 - protein, 13 aa, Artificial sequence
  TCR-CDR3 peptide sequence
CASSSGQKNTLYF

SEQ ID NO: 1885 - protein, 14 aa, Artificial sequence
  TCR-CDR3 peptide sequence
CASSSGQSQNTLYF

SEQ ID NO: 1886 - protein, 13 aa, Artificial sequence
  TCR-CDR3 peptide sequence
CASSSGQYAEQFF

SEQ ID NO: 1887 - protein, 14 aa, Artificial sequence
  TCR-CDR3 peptide sequence
CASSSGSSAETLYF

SEQ ID NO: 1888 - protein, 14 aa, Artificial sequence
  TCR-CDR3 peptide sequence
CASSSGTGAETLYF

SEQ ID NO: 1889 - protein, 14 aa, Artificial sequence
  TCR-CDR3 peptide sequence
CASSSLGDQDTQYF

SEQ ID NO: 1890 - protein, 13 aa, Artificial sequence
  TCR-CDR3 peptide sequence
CASSSNSAETLYF

SEQ ID NO: 1891 - protein, 11 aa, Artificial sequence
  TCR-CDR3 peptide sequence
CASSSNSDYTF

SEQ ID NO: 1892 - protein, 13 aa, Artificial sequence
  TCR-CDR3 peptide sequence
CASSSNSQNTLYF

SEQ ID NO: 1893 - protein, 14 aa, Artificial sequence
  TCR-CDR3 peptide sequence
CASSSQGNQDTQYF

SEQ ID NO: 1894 - protein, 12 aa, Artificial sequence
  TCR-CDR3 peptide sequence
CASSSQNTEVFF

SEQ ID NO: 1895 - protein, 13 aa, Artificial sequence
  TCR-CDR3 peptide sequence
CASSSQNYAEQFF

SEQ ID NO: 1896 - protein, 14 aa, Artificial sequence
  TCR-CDR3 peptide sequence
CASSSTGSQNTLYF

SEQ ID NO: 1897 - protein, 12 aa, Artificial sequence
  TCR-CDR3 peptide sequence
CASSSTNSDYTF

SEQ ID NO: 1898 - protein, 14 aa, Artificial sequence
  TCR-CDR3 peptide sequence
CASSSWGSQNTLYF

SEQ ID NO: 1899 - protein, 13 aa, Artificial sequence
  TCR-CDR3 peptide sequence
CASSTGNYAEQFF

SEQ ID NO: 1900 - protein, 13 aa, Artificial sequence
  TCR-CDR3 peptide sequence
CASSTGSAETLYF

SEQ ID NO: 1901 - protein, 13 aa, Artificial sequence
  TCR-CDR3 peptide sequence
CASSWGGQNTLYF

SEQ ID NO: 1902 - protein, 14 aa, Artificial sequence
  TCR-CDR3 peptide sequence
CASSWTANTGQLYF

SEQ ID NO: 1903 - protein, 12 aa, Artificial sequence
  TCR-CDR3 peptide sequence
CASSYGAETLYF

SEQ ID NO: 1904 - protein, 13 aa, Artificial sequence
  TCR-CDR3 peptide sequence
CASSYGGQNTLYF

SEQ ID NO: 1905 - protein, 12 aa, Artificial sequence
  TCR-CDR3 peptide sequence
CASSYNNQAPLF

SEQ ID NO: 1906 - protein, 13 aa, Artificial sequence
  TCR-CDR3 peptide sequence
CASSYQANTEVFF

SEQ ID NO: 1907 - protein, 12 aa, Artificial sequence
  TCR-CDR3 peptide sequence
CASSYRNTEVFF

SEQ ID NO: 1908 - protein, 14 aa, Artificial sequence
  TCR-CDR3 peptide sequence
CASSYWGNYAEQFF

SEQ ID NO: 1909 - protein, 13 aa, Artificial sequence
  TCR-CDR3 peptide sequence
CAWSLGGQNTLYF

SEQ ID NO: 1910 - protein, 13 aa, Artificial sequence
  TCR-CDR3 peptide sequence
CAWSLGNYAEQFF

SEQ ID NO: 1911 - protein, 14 aa, Artificial sequence
  TCR-CDR3 peptide sequence
CAWSLGSSAETLYF

SEQ ID NO: 1912 - protein, 11 aa, Artificial sequence
  TCR-CDR3 peptide sequence
CAWSLGYEQYF

SEQ ID NO: 1913 - protein, 13 aa, Artificial sequence
  TCR-CDR3 peptide sequence
CGARDNSGNTLYF

SEQ ID NO: 1914 - protein, 13 aa, Artificial sequence
  TCR-CDR3 peptide sequence
CGARDSSQNTLYF

SEQ ID NO: 1915 - protein, 14 aa, Artificial sequence
  TCR-CDR3 peptide sequence
CGARDSSYNSPLYF

SEQ ID NO: 1916 - protein, 14 aa, Artificial sequence
  TCR-CDR3 peptide sequence
CGARDWGSSYEQYF

SEQ ID NO: 1917 - protein, 12 aa, Artificial sequence
  TCR-CDR3 peptide sequence
CGARQGNTEVFF

SEQ ID NO: 1918 - protein, 13 aa, Artificial sequence
  TCR-CDR3 peptide sequence
CGARTVSNERLFF

SEQ ID NO: 1919 - protein, 9 aa, Artificial sequence
  TCR-CDR3 peptide sequence
CSADAEQFF

SEQ ID NO: 1920 - protein, 10 aa, Artificial sequence
  TCR-CDR3 peptide sequence
CSAGNSDYTF

SEQ ID NO: 1921 - protein, 11 aa, Artificial sequence
  TCR-CDR3 peptide sequence
CSAGQNTEVFF

SEQ ID NO: 1922 - protein, 11 aa, Artificial sequence
  TCR-CDR3 peptide sequence
CSAGSAETLYF

SEQ ID NO: 1923 - protein, 12 aa, Artificial sequence
  TCR-CDR3 peptide sequence
CASSQSRYEQYF

SEQ ID NO: 1924 - protein, 15 aa, Artificial sequence
  TCR-CDR3 peptide sequence
CASSLGTTNTGQLYF

SEQ ID NO: 1925 - protein, 14 aa, Artificial sequence
  TCR-CDR3 peptide sequence
CASSLGGNQDTQYF

SEQ ID NO: 1926 - protein, 15 aa, Artificial sequence
  TCR-CDR3 peptide sequence
CASGDAGAANSDYTF

SEQ ID NO: 1927 - protein, 14 aa, Artificial sequence
  TCR-CDR3 peptide sequence
CASGDAGAAAEQFF

SEQ ID NO: 1928 - protein, 14 aa, Artificial sequence
  TCR-CDR3 peptide sequence
CASGDAGAAETLYF

SEQ ID NO: 1929 - protein, 13 aa, Artificial sequence
  TCR-CDR3 peptide sequence
CASGDRDYAEQFF

SEQ ID NO: 1930 - protein, 13 aa, Artificial sequence
  TCR-CDR3 peptide sequence
CASSDGQYAEQFF

SEQ ID NO: 1931 - protein, 13 aa, Artificial sequence
  TCR-CDR3 peptide sequence
CASSGDRDAEQFF

SEQ ID NO: 1932 - protein, 15 aa, Artificial sequence
  TCR-CDR3 peptide sequence
CASSDDRGGGTEVFF

SEQ ID NO: 1933 - protein, 15 aa, Artificial sequence
  TCR-CDR3 peptide sequence
CASSQDPGGAETLYF

SEQ ID NO: 1934 - protein, 14 aa, Artificial sequence
  TCR-CDR3 peptide sequence
CASSQGDWGYEQYF

SEQ ID NO: 1935 - protein, 15 aa, Artificial sequence
  TCR-CDR3 peptide sequence
CASSSLGGNQDTQYF

SEQ ID NO: 1936 - protein, 13 aa, Artificial sequence
  TCR-CDR3 peptide sequence
CASRLGNQDTQYF

SEQ ID NO: 1937 - protein, 12 aa, Artificial sequence
  TCR-CDR3 peptide sequence
CGAGQGGNTLYF

SEQ ID NO: 1938 - protein, 14 aa, Artificial sequence
  TCR-CDR3 peptide sequence
CASGDAGGGYEQYF

SEQ ID NO: 1939 - protein, 15 aa, Artificial sequence
  TCR-CDR3 peptide sequence
CASSLDWDNQDTQYF

SEQ ID NO: 1940 - protein, 14 aa, Artificial sequence
  TCR-CDR3 peptide sequence
CASSQVGNQDTQYF

SEQ ID NO: 1941 - protein, 14 aa, Artificial sequence
  TCR-CDR3 peptide sequence
CASRLTSSAETLYF

SEQ ID NO: 1942 - protein, 14 aa, Artificial sequence
  TCR-CDR3 peptide sequence
CASRLTSYNSPLYF

SEQ ID NO: 1943 - protein, 12 aa, Artificial sequence
  TCR-CDR3 peptide sequence
CASSFGDSDYTF

SEQ ID NO: 1944 - protein, 12 aa, Artificial sequence
  TCR-CDR3 peptide sequence
CASSHRASDYTF

SEQ ID NO: 1945 - protein, 12 aa, Artificial sequence
  TCR-CDR3 peptide sequence
CASSFGGSDYTF

SEQ ID NO: 1946 - protein, 12 aa, Artificial sequence
  TCR-CDR3 peptide sequence
CASSFEGSDYTF

SEQ ID NO: 1947 - protein, 12 aa, Artificial sequence
  TCR-CDR3 peptide sequence
CASGTGGSDYTF

SEQ ID NO: 1948 - protein, 14 aa, Artificial sequence
  TCR-CDR3 peptide sequence
CASSGTVNNQAPLF

SEQ ID NO: 1949 - protein, 14 aa, Artificial sequence
  TCR-CDR3 peptide sequence
CASSFTNNNQAPLF

SEQ ID NO: 1950 - protein, 14 aa, Artificial sequence
  TCR-CDR3 peptide sequence
CASSFPGNNQAPLF

SEQ ID NO: 1951 - protein, 14 aa, Artificial sequence
  TCR-CDR3 peptide sequence
CASRDGSYNSPLYF

SEQ ID NO: 1952 - protein, 14 aa, Artificial sequence
  TCR-CDR3 peptide sequence
CASSSTGYYAEQFF

SEQ ID NO: 1953 - protein, 15 aa, Artificial sequence
  TCR-CDR3 peptide sequence
CASSLSTGYYAEQFF

SEQ ID NO: 1954 - protein, 14 aa, Artificial sequence
  TCR-CDR3 peptide sequence
CASSSGGGYAEQFF

SEQ ID NO: 1955 - protein, 14 aa, Artificial sequence
  TCR-CDR3 peptide sequence
CASSPGQTYAEQFF

SEQ ID NO: 1956 - protein, 14 aa, Artificial sequence
  TCR-CDR3 peptide sequence
CASSLSGGGTEVFF

SEQ ID NO: 1957 - protein, 13 aa, Artificial sequence
  TCR-CDR3 peptide sequence
CASSIRDKNTLYF

SEQ ID NO: 1958 - protein, 16 aa, Artificial sequence
  TCR-CDR3 peptide sequence
CASSLGLGVGAETLYF

SEQ ID NO: 1959 - protein, 14 aa, Artificial sequence
  TCR-CDR3 peptide sequence
CASSWDRNYAEQFF

SEQ ID NO: 1960 - protein, 13 aa, Artificial sequence
  TCR-CDR3 peptide sequence
CASSGTGDDTQYF

SEQ ID NO: 1961 - protein, 13 aa, Artificial sequence
  TCR-CDR3 peptide sequence
CASSGTGRDTQYF

SEQ ID NO: 1962 - protein, 14 aa, Artificial sequence
  TCR-CDR3 peptide sequence
FCASSGGGQDTQYF

SEQ ID NO: 1963 - protein, 13 aa, Artificial sequence
  TCR-CDR3 peptide sequence
CASSGQGQDTQYF

SEQ ID NO: 1964 - protein, 13 aa, Artificial sequence
  TCR-CDR3 peptide sequence
CASSGLGQDTQYF

SEQ ID NO: 1965 - protein, 13 aa, Artificial sequence
  TCR-CDR3 peptide sequence
CASSGQGEDTQYF

SEQ ID NO: 1966 - protein, 14 aa, Artificial sequence
  TCR-CDR3 peptide sequence
CASSILGDTGQLYF

SEQ ID NO: 1967 - protein, 13 aa, Artificial sequence
  TCR-CDR3 peptide sequence
CASSTTGTYEQYF

SEQ ID NO: 1968 - protein, 14 aa, Artificial sequence
  TCR-CDR3 peptide sequence
CASSDEGGQNTLYF

SEQ ID NO: 1969 - protein, 14 aa, Artificial sequence
  TCR-CDR3 peptide sequence
CASSIWGNYAEQFF

SEQ ID NO: 1970 - protein, 12 aa, Artificial sequence
  TCR-CDR3 peptide sequence
CASSDAREPQYF

SEQ ID NO: 1971 - protein, 13 aa, Artificial sequence
  TCR-CDR3 peptide sequence
CASSGRNTGQLYF

SEQ ID NO: 1972 - protein, 13 aa, Artificial sequence
  TCR-CDR3 peptide sequence
CGASDSSGNTLYF

SEQ ID NO: 1973 - protein, 12 aa, Artificial sequence
  TCR-CDR3 peptide sequence
CASKTDGNTLYF

SEQ ID NO: 1974 - protein, 15 aa, Artificial sequence
  TCR-CDR3 peptide sequence
CASSIGTGGYAEQFF

SEQ ID NO: 1975 - protein, 14 aa, Artificial sequence
  TCR-CDR3 peptide sequence
CASSDGWGQNTLYF

SEQ ID NO: 1976 - protein, 13 aa, Artificial sequence
  TCR-CDR3 peptide sequence
CASSDWGGYEQYF

SEQ ID NO: 1977 - protein, 14 aa, Artificial sequence
  TCR-CDR3 peptide sequence
CASSQEGGNTEVFF

SEQ ID NO: 1978 - protein, 13 aa, Artificial sequence
  TCR-CDR3 peptide sequence
CASSDAGNYEQYF

SEQ ID NO: 1979 - protein, 13 aa, Artificial sequence
  TCR-CDR3 peptide sequence
CASSAGGADTQYF

SEQ ID NO: 1980 - protein, 15 aa, Artificial sequence
  TCR-CDR3 peptide sequence
CASSPGQGDTGQLYF

SEQ ID NO: 1981 - protein, 15 aa, Artificial sequence
  TCR-CDR3 peptide sequence
CASSSTWGNYAEQFF

SEQ ID NO: 1982 - protein, 12 aa, Artificial sequence
  TCR-CDR3 peptide sequence
CGARESSYEQYF

SEQ ID NO: 1983 - protein, 12 aa, Artificial sequence
  TCR-CDR3 peptide sequence
CGARSSQNTLYF

SEQ ID NO: 1984 - protein, 15 aa, Artificial sequence
  TCR-CDR3 peptide sequence
CASSQDWGGSYEQYF

SEQ ID NO: 1985 - protein, 14 aa, Artificial sequence
  TCR-CDR3 peptide sequence
CASSFWGGDAEQFF

SEQ ID NO: 1986 - protein, 16 aa, Artificial sequence
  TCR-CDR3 peptide sequence
CASSQGTGGNTGQLYF

SEQ ID NO: 1987 - protein, 14 aa, Artificial sequence
  TCR-CDR3 peptide sequence
CASSQDANTGQLYF

SEQ ID NO: 1988 - protein, 14 aa, Artificial sequence
  TCR-CDR3 peptide sequence
CASSITDTNTEVFF

SEQ ID NO: 1989 - protein, 15 aa, Artificial sequence
  TCR-CDR3 peptide sequence
CASSQDYRANTEVFF

SEQ ID NO: 1990 - protein, 15 aa, Artificial sequence
  TCR-CDR3 peptide sequence
CASSQPGQSNERLFF

SEQ ID NO: 1991 - protein, 15 aa, Artificial sequence
  TCR-CDR3 peptide sequence
CASSQDGTANERLFF

SEQ ID NO: 1992 - protein, 14 aa, Artificial sequence
  TCR-CDR3 peptide sequence
CASSQVGYNSPLYF

SEQ ID NO: 1993 - protein, 15 aa, Artificial sequence
  TCR-CDR3 peptide sequence
CASSQLGQNYAEQFF

SEQ ID NO: 1994 - protein, 14 aa, Artificial sequence
  TCR-CDR3 peptide sequence
CASSQEMGENTLYF

SEQ ID NO: 1995 - protein, 14 aa, Artificial sequence
  TCR-CDR3 peptide sequence
CASSQQGYNSPLYF

SEQ ID NO: 1996 - protein, 14 aa, Artificial sequence
  TCR-CDR3 peptide sequence
CASSVTGSNTEVFF

SEQ ID NO: 1997 - protein, 14 aa, Artificial sequence
  TCR-CDR3 peptide sequence
CASSPTGRNTEVFF

SEQ ID NO: 1998 - protein, 12 aa, Artificial sequence
  TCR-CDR3 peptide sequence
CASGDGNQAPLF

SEQ ID NO: 1999 - protein, 16 aa, Artificial sequence
  TCR-CDR3 peptide sequence
CASSFRDRGNYAEQFF

SEQ ID NO: 2000 - protein, 15 aa, Artificial sequence
  TCR-CDR3 peptide sequence
CASSSGGSNTGQLYF

SEQ ID NO: 2001 - protein, 16 aa, Artificial sequence
  TCR-CDR3 peptide sequence
CASSQSGGSNTGQLYF

SEQ ID NO: 2002 - protein, 13 aa, Artificial sequence
  TCR-CDR3 peptide sequence
CASGGANTGQLYF

SEQ ID NO: 2003 - protein, 13 aa, Artificial sequence
  TCR-CDR3 peptide sequence
CASGGGNTGQLYF

SEQ ID NO: 2004 - protein, 15 aa, Artificial sequence
  TCR-CDR3 peptide sequence
CASSRGGANTGQLYF

SEQ ID NO: 2005 - protein, 15 aa, Artificial sequence
  TCR-CDR3 peptide sequence
CASSRGGGNTGQLYF

SEQ ID NO: 2006 - protein, 16 aa, Artificial sequence
  TCR-CDR3 peptide sequence
CAWSLKGGANTGQLYF

SEQ ID NO: 2007 - protein, 14 aa, Artificial sequence
  TCR-CDR3 peptide sequence
CASSARTANTEVFF

SEQ ID NO: 2008 - protein, 15 aa, Artificial sequence
  TCR-CDR3 peptide sequence
CASSFHISYNSPLYF

SEQ ID NO: 2009 - protein, 13 aa, Artificial sequence
  TCR-CDR3 peptide sequence
CASSFGVNSDYTF

SEQ ID NO: 2010 - protein, 13 aa, Artificial sequence
  TCR-CDR3 peptide sequence
CASSLGTGGKQFF

SEQ ID NO: 2011 - protein, 13 aa, Artificial sequence
  TCR-CDR3 peptide sequence
CASSPGTGQAPLF

SEQ ID NO: 2012 - protein, 13 aa, Artificial sequence
  TCR-CDR3 peptide sequence
CASSDQANTEVFF

SEQ ID NO: 2013 - protein, 14 aa, Artificial sequence
  TCR-CDR3 peptide sequence
CASSDAGANTEVFF

SEQ ID NO: 2014 - protein, 13 aa, Artificial sequence
  TCR-CDR3 peptide sequence
CASSDNANSDYTF

SEQ ID NO: 2015 - protein, 13 aa, Artificial sequence
  TCR-CDR3 peptide sequence
CASSDAGHSPLYF

SEQ ID NO: 2016 - protein, 15 aa, Artificial sequence
  TCR-CDR3 peptide sequence
CASSQDWGFQDTQYF

SEQ ID NO: 2017 - protein, 14 aa, Artificial sequence
  TCR-CDR3 peptide sequence
CAWSRTGGNSDYTF

SEQ ID NO: 2018 - protein, 12 aa, Artificial sequence
  TCR-CDR3 peptide sequence
CASSLGISDYTF

SEQ ID NO: 2019 - protein, 13 aa, Artificial sequence
  TCR-CDR3 peptide sequence
CASSSGGSDGYTF

SEQ ID NO: 2020 - protein, 11 aa, Artificial sequence
  TCR-CDR3 peptide sequence
CASSKANSYTF

SEQ ID NO: 2021 - protein, 15 aa, Artificial sequence
  TCR-CDR3 peptide sequence
CASSLGTTNTGELFF

SEQ ID NO: 2022 - protein, 14 aa, Artificial sequence
  TCR-CDR3 peptide sequence
CASTDAGGSYEQYF

SEQ ID NO: 2023 - protein, 13 aa, Artificial sequence
  TCR-CDR3 peptide sequence
CASSGTDQETQYF

SEQ ID NO: 2024 - protein, 14 aa, Artificial sequence
  TCR-CDR3 peptide sequence
CASSRTANTGELFF

SEQ ID NO: 2025 - protein, 13 aa, Artificial sequence
  TCR-CDR3 peptide sequence
CASSKGGQNTIYF

SEQ ID NO: 2026 - protein, 14 aa, Artificial sequence
  TCR-CDR3 peptide sequence
CASSGQGNYNEQFF

SEQ ID NO: 2027 - protein, 14 aa, Artificial sequence
  TCR-CDR3 peptide sequence
CASSPGQSNEKLFF

SEQ ID NO: 2028 - protein, 13 aa, Artificial sequence
  TCR-CDR3 peptide sequence
CASSLEGDTEAFF

SEQ ID NO: 2029 - protein, 12 aa, Artificial sequence
  TCR-CDR3 peptide sequence
CASSHSGNTIYF

SEQ ID NO: 2030 - protein, 13 aa, Artificial sequence
  TCR-CDR3 peptide sequence
CASSGTANTEAFF

SEQ ID NO: 2031 - protein, 13 aa, Artificial sequence
  TCR-CDR3 peptide sequence
CASSGTGLDTQYF

SEQ ID NO: 2032 - protein, 12 aa, Artificial sequence
  TCR-CDR3 peptide sequence
CASSGLGDTQYF

SEQ ID NO: 2033 - protein, 9 aa, Artificial Sequence
  TCR-CDR3 peptide sequence
ASSLGGNQD

SEQ ID NO: 2034 - protein, 8 aa, Artificial Sequence
  TCR-CDR3 peptide sequence
ASRLGNQD

SEQ ID NO: 2035 - protein, 11 aa, Artificial Sequence
  TCR-CDR3 peptide sequence
ASSLGLGANQD

SEQ ID NO: 2036 - protein, 9 aa, Artificial Sequence
  TCR-CDR3 peptide sequence
ASSLGANQD

(19) United States
(12) Patent Application Publication
(10) Pub. No.: US 2017/0174764 A1
(43) Pub. Date: Jun. 22, 2017

(54) T-CELL RECEPTOR CDR3 PEPTIDES AND ANTIBODIES

(71) Applicant: Yeda Research and Development Co. Ltd., Rehovot (IL)

(72) Inventors: Irun R. COHEN, Rehovot (IL); Nir FRIEDMAN, Rehovot (IL); Asaf MADI, Rehovot (IL); Eric SHIFRUT, Rehovot (IL); Shlomit REICH-ZELIGER, Rehovot (IL); Asaf PORAN, Rehovot (IL)

(21) Appl. No.: 15/127,023

(22) PCT Filed: Mar. 26, 2015
(86) PCT No.: PCT/IL15/50329

§ 371 Date: Sep. 18, 2016

Related U.S. Application Data
(60) Provisional application No. 61/970,933, filed on Mar. 27, 2014.

(51) Int. Cl. C07K 16/28

(52) U.S. Cl. CPC C07K 16/2809; A61K 2039/505

(57) ABSTRACT

The present invention provides isolated peptides derived from TCR CDR3 segments, antibodies which recognize them, pharmaceutical compositions comprising them and methods of their use for modulating self-immunity.

### FIELD OF THE INVENTION

The invention relates to peptides derived from T-cell receptor (TCR) CDR3 segment related to self-immunity, and to antibodies to these peptide sequences. The invention also relates to methods of use of specific peptides for prevention, suppression and treatment of autoimmune diseases and allograft rejection. Also provided are antibodies specific to several CDR3 derived peptides for tumor immunotherapy and against pathogens.

### BACKGROUND OF THE INVENTION

The potential diversity of TCR molecules synthesized during the maturation of T cells in the thymus is estimated to be >10′⁵ for the mouse TCRαβ repertoire and >10¹⁰ for the TCRβ segment of the TCR. In contrast, the number of unique TCR types appearing in the peripheral lymphoid organs of an individual mouse (˜10⁶) is many orders of magnitude less than this potential diversity. This excess of potential thymic TCR diversity leads to the expectation that different individuals would hardly ever share the same TCR recombination. Nevertheless, several reports have demonstrated identical TCR sequences occurring in the T-cell responses to defined antigens in different MHC-matched humans (V. P. Argaet et al., J Exp Med 180, 2335, 1994; P. A. Moss et al., Proc Natl Acad Sci USA 88, 8987, 1991), macaques (V. Venturi, et al., J Immunol 181, 2597, 2008) and mice (V. Venturi, et al., Nat Rev Immunol 8, 231, 2008). There have also been studies reporting substantial overlap in the naive TCR repertoire between two mice, of about 18-27%. Shared TCR molecules can be referred to as public; private T-cell responses involve little TCR sharing. It has been suggested that an adequate sampling of individual TCR repertoires would demonstrate the true prevalence of public TCR sequences (V. Venturi et al., Proc Natl Acad Sci USA 103, 18691, 2006).

T cell activation plays an important role in specific responses against pathogens, in tumor immunity and in autoimmune and inflammatory disorders. Therefore, methods of modulating the immune response and the T cell response in particular, are widely used in a plethora of medical conditions.

Tumor cells, for example, express many antigens that differ from those of healthy cells and against which the healthy immune system is posed to respond. Despite existing immunity to tumor-associated antigens, tumors can evade immune rejection by activating immune suppressor T cells of various types including CD4+ regulatory T cells (Tregs); growing tumors attract these immune suppressor cells, which down-regulate effector T cells and other immune cells that could otherwise reject the tumor. The tumor, in other words, hijacks immune regulation mechanisms that normally serve to prevent or down-regulate potential autoimmune effector reactions that might otherwise cause an autoimmune disease. The successful tumor masquerades as a normal cell population, not attacked by the immune system, despite the fact that it expresses tumor-associated antigens—body molecules that are abnormal in their structure, tissue site, or developmental timing. This new understanding of the tumor-immune relationship has led to the development of new therapies aimed at depriving the tumor of its protective immune suppression. A proof-of-concept has been demonstrated by the use of anti-PD1 and anti CTLA-4 antibodies in tumor immunotherapy (Curran M A, et al., PNAS, 107(9):4275-80, 2010); these antibodies target and disarm immune regulatory mechanisms, and thereby unleash quiescent or suppressed tumor-associated autoimmunity to attack the tumor with a destructive, autoimmune-like reaction. Initial clinical trials have been quite encouraging and major pharmaceutical companies are racing ahead to complete the development of anti-PD1 immunotherapy (Wolchok J D et al., N Engl J Med. 369(2):122-33, 2013). The disadvantage of anti-PD1 and anti-CTLA-4 treatment is that it lacks specificity; for example, the PD1 molecule is expressed on all T cells, B cells and macrophages. Specific treatment should target suppressor T cells that are specifically associated with the tumor, to reduce side effects and increase efficacy.

TCR diversity has been an obstacle for treatments such as T-cell vaccination based on specific TCR sequences. This might be alleviated if public TCRs can be used as effective T-cell vaccines. There is an unmet need to provide effective compositions for prevention, suppression and treatment of autoimmune diseases and allograft rejection and new, effective and specific therapies for cancer and against pathogens.

### SUMMARY OF THE INVENTION

The present invention is based in part on high throughput study of the TCR repertoire and provides new therapeutic peptides for prevention and treatment of autoimmunity and allo-immunity, and neutralizing antibodies to promote immunity against pathogens and for cancer immunotherapy.

The proposed peptides and antibodies of the present invention emerged from the discovery of a set of T cells expressing public TCR molecules featuring CDR3 segments that are highly shared among individual mice, monkeys and humans. These public T cells represent some 5-10% of the T cell repertoire. Functionally, the public set of T cells is enriched for T cells associated with autoimmunity, with allograft immunity, and, with tumor-infiltrating T cells and T cells responsive to tumor-associated antigens such as MDM2 and HSP60. Indeed, CDR3 segments associated with tumor-related T cells are shared by humans and mice. It is thus plausible that experimental results obtained in mice are relevant to humans. Experiments in mice shown herein indicate that an antibody raised against a CDR3 peptide expressed by a relatively public T cell clonotype can activate a latent autoimmune T cell effector response in a Diabetes Type I mouse model and conversely inhibit tumor progression in a lung carcinoma mouse tumor model.

According to an aspect of some embodiments of the present invention there is provided an isolated agent capable of at least one of:

(i) binding a TCR presented on a T cell;

(ii) competing with binding of a TCR presented on a T cell to a target of the T cell;

(iii) eliciting a specific immune-response of a T cell; and

(iv) eliciting a specific immune-response against a T cell;

wherein the T cell is expressing a TCR-CDR3 sequence comprising an amino acid sequence selected from the group consisting of SEQ ID NOs of Table 8, wherein when the agent is a peptide it is selected from the group consisting of SEQ ID NOs of Table 7.

According to an aspect of some embodiments of the present invention there is provided an isolated peptide of no more than 20 amino acids comprising an amino acid sequence having a CDR3 sequence of a TCR on a T cell, the CDR3 sequence being selected from the group consisting of SEQ ID NOs of Table 7.

According to an aspect of some embodiments of the present invention there is provided a use of:

(i) the isolated agent;

(ii) the isolated peptide; or

(iii) an isolated peptide of no more than 20 amino acids comprising an amino acid sequence having a CDR3 sequence of a TCR on a T cell, the CDR3 sequence being selected from the group consisting of SEQ ID NOs of Table 8,

in the manufacture of a medicament identified for treating a disease associated with the T cell.

According to an aspect of some embodiments of the present invention there is provided a method of treating a disease associated with the T cell, the method comprising administering to a subject in need thereof an effective amount of:

(i) the isolated agent;

(ii) the isolated peptide; or

(iii) an isolated peptide of no more than 20 amino acids comprising an amino acid sequence having a CDR3 sequence of a TCR on a T cell, the CDR3 sequence being selected from the group consisting of SEQ ID NOs of Table 8,

thereby treating the disease associated with the T cell.

According to some embodiments of the invention, the agent is selected from the group consisting of antibody, T cell, peptide and polynucleotide. According to an aspect of some embodiments of the present invention there is provided an isolated antibody comprising an antigen recognition domain capable of specifically binding SEQ ID NO: 1 of a TCR presented on a T cell.

According to some embodiments of the invention, there is provided a use of the isolated antibody in the manufacture of a medicament identified for treating a disease associated with the T cell.

According to some embodiments of the invention, there is provided a method of treating a disease associated with a T cell expressing a TCR-CDR3 segment comprising an amino acid sequence of SEQ ID NO: 1 in a subject in need thereof, the method comprising administering to the subject a therapeutically effective amount of the isolated antibody, thereby treating the disease associated with a T cell expressing the TCR-CDR3 segment comprising an amino acid sequence of SEQ ID NO: 1 in the subject.

According to some embodiments of the invention, the T cell is a regulatory T cell.

According to some embodiments of the invention, the T cell is an effector T cell.

The present invention provides, according to a further aspect an isolated peptide of 8-20 amino acids, or an analog thereof, comprising a sequence of at least 6 contiguous amino acids derived from a TCR-CDR3 segment, wherein the peptide does not comprise a sequence selected from the group consisting of: ASSLGGNQD (SEQ ID NO: 2033); ASRLGNQD (SEQ ID NO: 2034); ASSLGLGANQD (SEQ ID NO: 2035); and ASSLGANQD (SEQ ID NO: 2036).

According to some embodiments, the CDR3 segment is from beta TCR.

According to some embodiments, the isolated peptide comprises an amino acid sequence which was found to be associated with immunity selected from the group consisting of: autoimmunity, pathogenic immunity, tumor immunity and, graft rejection, and was further identified in at least 75% of tested mammalian individuals.

According to other embodiments, the isolated peptide comprises an amino acid sequence which was found to be associated with immunity selected from the group consisting of: autoimmunity, pathogenic immunity, tumor immunity and, graft rejection, and was further identified in human individuals.

According to other embodiments, the isolated peptide comprises an amino acid sequence which was found to be associated with immunity selected from the group consisting of: autoimmunity, pathogenic immunity, tumor immunity and, graft rejection, was further identified in at least 75% of tested mammalian individuals, and was identified also in human individuals.

According to other embodiments, the isolated peptide comprises an amino acid sequence that was identified in at least 75% of tested mammalian individuals, and was identified also in human individuals.

According to some embodiments, the peptide or peptide analog consists of 10-16 amino acids.

According to some embodiments, the isolated peptide or analog thereof comprises 8-20 (e.g., 8-14) contiguous amino acids derived from a TCR-CDR3 segment.

According to some embodiments, the TCR-CDR3 segment is from mouse.

According to some embodiments, the TCR-CDR3 segment is from human.

According to some embodiments, the TCR-CDR3 segment is shared by human and mouse.

According to a specific embodiment, the CDR3 sequence is selected from any of the tables provided hereinbelow.

According to some embodiments of the invention, the CDR3 sequence is selected from the group consisting of the sequences in Table 2.

According to some embodiments of the invention, the peptide amino acid sequence is selected from the group consisting of SEQ ID NOs of Table 2.

TABLE 2

| SEQ | | sharing |
|---|---|---|
| ID | | level in |
| NO: | sequence | mice |
| 2 | CASSGTGQDTQYF | 27 |
| 3 | CASSGTGEDTQYF | 25 |
| 4 | CASSGLGEDTQYF | 24 |
| 5 | CASGGYEQYF | 23 |

Each possibility represents a separate embodiment of the present invention.

According to some embodiments of the invention, the CDR3 sequence is selected from the group consisting of the sequences in Table 3.

According to some embodiments of the invention, the peptide amino acid sequence is selected from the group consisting of SEQ ID NOs of Table 3.

TABLE 3

| SEQ | | sharing |
|---|---|---|
| ID | | level in |
| NO: | sequence | mice |
| 6 | CASSPGGSYEQYF | 20 |
| 7 | CASSSRYEQYF | 17 |
| 8 | CASSGTGKDTQYF | 17 |
| 9 | CASSFGVSYEQYF | 12 |
| 10 | CASSRGSYEQYF | 11 |
| 11 | CASSPGTGVEQYF | 9 |
| 12 | CASSFGTGYEQYF | 9 |
| 13 | CASSGGAYEQYF | 6 |
| 14 | CASSLGVGDTQYF | 5 |
| 15 | CASTGTGQDTQYF | 5 |
| 16 | CASSGRGQDTQYF | 4 |
| 17 | CASGGAYEQYF | 2 |
| 18 | CASSFVGSYEQYF | 1 |
| 19 | CASSRRPYEQYF | 1 |

Each possibility represents a separate embodiment of the present invention.

According to some embodiments of the invention, the CDR3 sequence is selected from the group consisting of the sequences in Table 4.

According to some embodiments of the invention, the peptide amino acid sequence is selected from the group consisting of SEQ ID NOs of Table 4.

TABLE 4

| SEQ | | sharing |
|---|---|---|
| ID | | level in |
| NO: | Sequence | mice |
| 20 | CASSLGGQNTLYF | 28 |
| 21 | CASSLGNSDYTF | 28 |
| 22 | CASSSANSDYTG | 27 |
| 23 | CASSSGNSDYTG | 27 |
| 24 | CASSGTANTEVFF | 26 |
| 25 | CASSGQGNYAEQFF | 26 |
| 26 | CASGDWGYEQYF | 25 |
| 27 | CASGDAGGSYEQYF | 25 |
| 28 | CASSDGANTEVFF | 24 |
| 29 | CASSHSGNTLYF | 23 |
| 30 | CASSGTDQDTQYF | 23 |
| 31 | CASSPGQSNERLFF | 23 |
| 32 | CASSRTANTGQLYF | 23 |
| 33 | CASSDSANTEVFF | 22 |
| 34 | CASSLEGDTEVFF | 22 |

Each possibility represents a separate embodiment of the present invention.

According to some embodiments of the present invention, the CDR3 sequence is selected from the group consisting of the sequences in Table 5.

According to some embodiments of the present invention, the peptide amino acid sequence is selected from the group consisting of SEQ ID NOs of Table 5.

TABLE 5

| | | sharing |
|---|---|---|
| SEQ | | level |
| ID | | in |
| NO: | Sequence | mice |
| 35 | CASSLEGEDTQYF | 28 |
| 36 | CASSPGQQDTQYF | 28 |
| 37 | CASSFQDTQYF | 28 |
| 38 | CASSRQQDTQAT | 28 |
| 39 | CASSRDSQDTQYF | 28 |
| 40 | CASSLQGYEQYF | 28 |
| 41 | CASSDSSYEQYF | 28 |
| 42 | CASSLGSSYEQYF | 28 |
| 43 | CASGDGDTQYF | 28 |
| 44 | CASGDYEQYF | 28 |
| 45 | CASSLEDTQYF | 28 |
| 46 | CASSLEGDEQYF | 28 |
| 47 | CASSLGYEQYF | 28 |
| 48 | CASSLGQYEQYF | 28 |
| 49 | CASSVDGSYEQYF | 28 |
| 50 | CASSPQDTQYF | 28 |
| 51 | CASSLDNYEQYF | 28 |
| 52 | CASSLEGYEQYF | 28 |
| 53 | CASSLDEQYF | 28 |
| 54 | CASSLEGNQDTQYF | 28 |
| 55 | CASSLEGDTQYF | 28 |
| 56 | CASSLDYEQYF | 28 |
| 57 | CASSLGDTQYF | 28 |
| 58 | CASSLGEQYF | 28 |
| 59 | CASSQDTQYF | 28 |
| 60 | CASSLDRYEQYF | 28 |
| 61 | CASSGTGGYEQYF | 28 |
| 62 | CASSSYEQYF | 28 |
| 63 | CASSSGQYEQYF | 28 |
| 64 | CASSLLGGAEQFF | 28 |
| 65 | CASSLDRDEQYF | 28 |
| 66 | CASSLDQDTQYF | 28 |
| 67 | CASSLEGSSYEQYF | 28 |
| 68 | CASSLGGYEQYF | 28 |
| 69 | CASSLGAEQFF | 28 |
| 70 | CASSLSGYEQYF | 28 |
| 71 | CASSSSYEQYF | 28 |
| 72 | CASSLGQDTQYF | 28 |
| 73 | CASSLGGQDTQYF | 28 |
| 74 | CASSFNQDTQYF | 28 |
| 75 | CASGQDTQYF | 28 |
| 76 | CASSRDWGYEQYF | 28 |
| 77 | CASSLDSYEQYF | 28 |
| 78 | CASSRQYEQYF | 28 |
| 79 | CASSLKDTQYF | 28 |
| 80 | CASSLGQNTEVFF | 28 |
| 81 | CASSFGTGDEQYF | 28 |
| 82 | CASSLGEDTQYF | 28 |
| 83 | CASSRQNQDTQYF | 28 |
| 84 | CASSPSSYEQYF | 28 |
| 85 | CASSPDSYEQYF | 28 |
| 86 | CASSRDNYEQYF | 28 |
| 87 | CASSRDSYEQYF | 28 |
| 88 | CASSLDRVEQYF | 28 |
| 89 | CASSLYAEQFF | 28 |
| 90 | CASSLDAEQFF | 28 |
| 91 | CASSDSYEQYF | 28 |
| 92 | CASSFGTEVFF | 28 |
| 93 | CASSPDNYEQYF | 28 |
| 94 | CASSPGQYEQYF | 28 |
| 95 | CASSLQDTQYF | 28 |
| 96 | CASSLRDTQYF | 28 |
| 97 | CASSLGDEQYF | 28 |
| 98 | CASSLVAEQFF | 28 |
| 99 | CASSFSYEQYF | 28 |
| 100 | CASSLSYEQYF | 28 |
| 101 | CASSDQDTQYF | 28 |
| 102 | CASSDAGDTQYF | 28 |
| 103 | CASGDSYEQYF | 28 |
| 104 | CASSLDTQYF | 28 |
| 105 | CASSFYAEQFF | 28 |
| 106 | CASGDEQYF | 28 |
| 107 | CASGDAYEQYF | 28 |
| 108 | CASSLYEQYF | 28 |
| 109 | CASSRDSSYEQYF | 28 |
| 110 | CASSIRDTQYF | 28 |
| 111 | CASSRTGYEQYF | 27 |
| 112 | CASSDAGYEQYF | 27 |
| 113 | CASSQQDTQYF | 27 |
| 114 | CASSGQQDTQYF | 27 |
| 115 | CASGEDTQYF | 27 |
| 116 | CASSRQDTQYF | 27 |
| 117 | CASSLGQQDTQYF | 27 |
| 118 | CASSGGEQYF | 27 |
| 119 | CASSYQDTQYF | 27 |
| 120 | CASSLQYEQYF | 27 |
| 121 | CASSSQDTQYF | 27 |
| 122 | CASSQDRDTEVFF | 27 |
| 123 | CASSNQDTQYF | 27 |
| 124 | CASSSSSYEQYF | 27 |
| 125 | CASSLEGAEQFF | 27 |
| 126 | CASSFGQYEQYF | 27 |
| 127 | CASSLTGDEQYF | 27 |
| 128 | CASSRQGYEQYF | 27 |
| 129 | CASSSTGYEQYF | 27 |
| 130 | CASSLDGYEQYF | 27 |
| 131 | CASSSNQDTQYF | 27 |
| 132 | CASSPTGDEQYF | 27 |
| 133 | CASSESAEQFF | 27 |
| 134 | CASSLDRGEQYF | 27 |
| 135 | CASSLGYAEQFF | 27 |
| 136 | CASSSQGYEQYF | 27 |
| 137 | CASSPSYEQYF | 27 |
| 138 | CASSPDRYEQYF | 27 |
| 139 | CASSPNQDTQYF | 27 |
| 140 | CASSLSSYEQYF | 27 |
| 141 | CASSLAGYEQYF | 27 |
| 142 | CASSLTGGYEQYF | 27 |
| 143 | CASSLDTYEQYF | 27 |
| 144 | CASSLGGSSYEQYF | 27 |
| 145 | CASGYEQYF | 27 |
| 146 | CASGDADTQYF | 27 |
| 147 | CASSLVGYEQYF | 27 |
| 148 | CASGDEDTQYF | 27 |
| 149 | CASSQGQYEQYF | 27 |
| 150 | CASSLDSSYEQYF | 27 |
| 151 | CASSPTGYEQYF | 27 |
| 152 | CASSQYEQYF | 27 |
| 153 | CASSRQGQDTQYF | 26 |
| 154 | CASSLQGAEQFF | 26 |
| 155 | CASSDNYEQYF | 26 |
| 156 | CASSDRGDTQYF | 26 |
| 157 | CASSDAEQFF | 26 |
| 158 | CASSGQYEQYF | 26 |
| 159 | CASSSGGQDTQYF | 26 |
| 160 | CASSLGGAEQFF | 26 |
| 161 | CASSPDAEQFF | 26 |
| 162 | CASSLGGSYEQYF | 26 |
| 163 | CASSLAGDEQYF | 26 |
| 164 | CASSLSGGYEQYF | 26 |
| 165 | CASSYNQDTQYF | 26 |
| 166 | CASSLEGEQYF | 26 |
| 167 | CASSRDRGYEQYF | 26 |
| 168 | CASSLGGEQYF | 26 |
| 169 | CASSLEEQYF | 26 |
| 170 | CASGDDEQYF | 26 |
| 171 | CASSRLPSYEQYF | 26 |
| 172 | CASSLAGGQDTQYF | 26 |
| 171 | CASSRTGGQDTQYF | 26 |
| 174 | CASSLEGQDTQYF | 26 |
| 175 | CASSLVGDEQYF | 26 |
| 176 | CASSPQGYEQYF | 26 |
| 177 | CASSSDSYEQYF | 26 |
| 178 | CASSRTGEDTQYF | 26 |
| 179 | CASSRTGGYEQYE | 26 |
| 180 | CASSLQGSSYEQYF | 26 |
| 181 | CASSLTGNTEVFF | 26 |
| 182 | CASSLGGDTQYF | 26 |
| 183 | CASSLDWGYEQYF | 26 |
| 184 | CASSPGQSSYEQYF | 26 |
| 185 | CASSYSYEQYF | 26 |
| 186 | CASSLVEQYF | 26 |
| 187 | CASSLDRGNTEVFF | 26 |
| 188 | CASSLLGYEQYF | 26 |
| 189 | CASSQSSYEQYF | 26 |
| 190 | CASSEGDTQYF | 26 |
| 191 | CASSDRDTQYF | 26 |
| 192 | CASSDGDTQYF | 26 |
| 193 | CASSFYEQYF | 26 |
| 194 | CASSYEQYF | 26 |
| 195 | CASSRQSSYEQYF | 26 |
| 196 | CASSRDREDTQYF | 26 |
| 197 | CASSRYEQYF | 26 |
| 198 | CASSPQGTEVFF | 26 |
| 199 | CASSPGQGQDTQYF | 25 |
| 200 | CASSGDSYEQYF | 25 |
| 201 | CASGDFYEQYF | 25 |
| 202 | CASSQGTQYF | 25 |
| 203 | CASSHQDTQYF | 25 |
| 204 | CASSHYEQYF | 25 |
| 205 | CASSVQDTQYF | 25 |
| 206 | CASSLRGYEQYF | 25 |
| 207 | CASSLEQYEQYF | 25 |
| 208 | CASSLEGGEQYF | 25 |
| 209 | CASSLGGDEQYF | 25 |
| 210 | CASSWDSSYEQYF | 25 |
| 211 | CASSLQGGEQYF | 25 |
| 212 | CASSLGSVEQYF | 25 |
| 213 | CASSLGDSSYEQYF | 25 |
| 214 | CASSLGVEQYF | 25 |
| 215 | CASSFGGQDTQYF | 25 |
| 216 | CASSLGVQDTQYF | 25 |
| 217 | CASSDWGSSYEQYF | 25 |
| 218 | CASSLEQDTQYF | 25 |
| 219 | CASSPDRDEQYF | 25 |
| 220 | CASSLRGDTQYF | 25 |
| 221 | CASSLAGGYEQYF | 25 |
| 222 | CASSLGLGYEQYF | 25 |
| 223 | CASSFDAEQFF | 25 |
| 224 | CASSLREQYF | 25 |
| 225 | CASSQQGYEQYF | 25 |
| 226 | CASSQGNQDTQYF | 25 |
| 227 | CASSRDRGIDTQYF | 25 |
| 228 | CASSLAQDTQYF | 25 |
| 229 | CASSRDRQDTQYF | 25 |
| 230 | CASSDEDTQYF | 25 |
| 231 | CASGDNYEQYF | 25 |
| 232 | CASSPIGGQDTQYF | 25 |
| 233 | CASSPYEQYF | 25 |
| 234 | CASSLNAEQFF | 25 |
| 235 | CASSFGDTQYF | 25 |
| 236 | CASSLQSSYEQYF | 25 |
| 237 | CASSPGQDTQYF | 25 |
| 238 | CASSPGQGNTEVFF | 25 |
| 239 | CASSLDRGYEQYF | 25 |
| 240 | CASSLNERLFF | 25 |
| 241 | CASSLLGGQDTQYF | 25 |
| 242 | CASSQEDTQYF | 25 |
| 243 | CASSLGLGQDTQYF | 25 |
| 244 | CASSLGLQDTQYF | 25 |
| 245 | CASSLQGDEQYF | 25 |
| 246 | CASSPGLGEDTQYF | 25 |
| 247 | CASSLLGQDTQYF | 25 |
| 248 | CASSLDGAEQFF | 25 |
| 249 | CASSQDRDSDYTF | 25 |
| 250 | CASSLTGEDTQYF | 25 |
| 251 | CASSPGNTLYF | 25 |
| 252 | CASGDRDEQYF | 25 |
| 253 | CASGDGEQYF | 25 |
| 254 | CASSLDKYEQYF | 24 |
| 255 | CASSRDNSYEQYF | 24 |
| 256 | CASSDAGGSYEQYF | 24 |
| 257 | CASSGTGDEQYF | 24 |
| 258 | CASSLVGAETLYF | 24 |
| 259 | CASSLGGEDTQYF | 24 |
| 260 | CASSLGDSDYTF | 24 |
| 261 | CASSLVQDTQYF | 24 |
| 262 | CASSTQDTQYF | 24 |
| 263 | CASSHSYEQYF | 74 |
| 264 | CASSSDRDEQYF | 24 |
| 265 | CASSLEDSYEQYF | 24 |
| 266 | CASSLQGDTQYF | 24 |
| 267 | CASSLPGQDTQYF | 24 |
| 268 | CASSLGQGYEQYF | 24 |
| 269 | CASSPTGNSDYTF | 24 |
| 270 | CASSRLGEDTQYF | 24 |
| 271 | CASSRTGGAETLYF | 24 |
| 272 | CASSLQGQDTQYF | 24 |
| 273 | CASSPGQGYEQYF | 24 |
| 274 | CASSPGDTQYF | 24 |
| 275 | CASSRSSYEQYF | 24 |
| 276 | CASSLSGDEQYF | 24 |
| 277 | CASSSGYEQYF | 24 |
| 278 | CASSQTGGQDTQYF | 24 |
| 279 | CASSLTGYEQYF | 24 |
| 280 | CASSLDIYEQYF | 74 |
| 281 | CASSLSQDTQYF | 24 |
| 282 | CASSLAGSSYEQYF | 24 |
| 283 | CASSEGGREQYF | 24 |
| 284 | CASSGTGDTQYF | 24 |
| 285 | CASSGTEDTQYF | 24 |
| 286 | CASSLTYEQYF | 24 |
| 287 | CASSLDDTQYF | 24 |
| 288 | CASSLGTEDTQYF | 24 |
| 289 | CSAEDTQYF | 24 |
| 290 | CASSRDIYEQYF | 74 |
| 291 | CASSLRAEQFF | 24 |
| 292 | CASSLLGEDTQYF | 24 |
| 293 | CASSFGAEQFF | 24 |
| 294 | CASSIQDTQYF | 24 |
| 295 | CASSIQYEQYF | 74 |
| 296 | CASSEGQYEQYF | 24 |
| 297 | CASSDRGYEQYF | 24 |
| 298 | CASGEGEQYF | 24 |
| 299 | CASSDYEQYF | 24 |
| 300 | CASSDAYEQYF | 74 |
| 301 | CASGEYEQYF | 24 |
| 302 | CASSLSGNTEVFF | 24 |
| 303 | CASSLVGEQYF | 24 |
| 304 | CASSIGQYEQYF | 24 |
| 305 | CASSIGDTQYF | 24 |
| 306 | CASSLDSAEQFF | 24 |
| 307 | CASSQDRNTEVFF | 24 |
| 308 | CASSEQDTQYF | 23 |
| 309 | CASSGQDTQYF | 23 |
| 310 | CASSLQGEQYF | 23 |
| 311 | CASSLDNSYEQYF | 23 |
| 312 | CASGDSSYEQYF | 23 |
| 313 | CASGDASYEQYF | 23 |
| 314 | CASSLTGGQDTQYF | 23 |
| 315 | CASSLEGGQDTQYF | 23 |
| 316 | CASSLTGGDTQYF | 23 |
| 317 | CASSFTGEDTQYF | 23 |
| 318 | CASSFRDTQYF | 23 |
| 319 | CASSLEAQFF | 23 |
| 320 | CASSLGNYEQYF | 23 |
| 321 | CASSRQGDTQYF | 23 |
| 322 | CASSSTGGYEQYF | 23 |
| 323 | CASSLGQSSYEQYF | 23 |
| 324 | CASSLGDSYEQYF | 23 |
| 325 | CASSWDSQDTQYF | 23 |
| 326 | CASSPRGQDTQYF | 23 |
| 327 | CASSLRSSYEQYF | 23 |
| 328 | CASSRLGYEQYF | 23 |
| 329 | CASSLPGGQDTQYF | 23 |
| 330 | CASSLEYEQYF | 23 |
| 331 | CASSSGSSYEQYF | 23 |
| 332 | CASSRGQYEQYF | 23 |
| 333 | CASSQGEQYF | 23 |
| 334 | CASSLDGDTQYF | 23 |
| 335 | CASRGQANTEVFF | 23 |
| 336 | CASSPPGQQDTQYF | 23 |
| 337 | CASSPGSSYEQYF | 23 |
| 338 | CASSRDQDTQYF | 23 |
| 339 | CASSRDFYEQYF | 23 |
| 340 | CASSSEDTQYF | 23 |
| 341 | CASSRDRYEQYF | 23 |
| 342 | CASSEGSSYEQYF | 23 |
| 343 | CASSLGDAEQFF | 23 |
| 344 | CASSQDQDTQYF | 23 |
| 345 | CASSPGTGQDTQYF | 23 |
| 346 | CASSRTGDQDTQYF | 23 |
| 347 | CASSLQGRDTQYF | 23 |
| 348 | CASSWTGEDTQYF | 23 |
| 349 | CASSWGYEQYF | 23 |
| 350 | CASSLRGQDTQYF | 23 |
| 351 | CASSLEGVEQYF | 23 |
| 352 | CASSFKDTQYF | 23 |
| 353 | CASSDEGYEQYF | 23 |
| 354 | CASSDADTQYF | 23 |
| 355 | CASSPDQDTQYF | 23 |
| 356 | CASSPGGQDTQYF | 23 |
| 357 | CASSLRQYEQYF | 23 |
| 358 | CASSLVSYEQYF | 23 |
| 359 | CASSSTGDEQYF | 23 |
| 360 | CSADSYEQYF | 23 |
| 361 | CASGEQYF | 23 |
| 362 | CASSPDWGYEQYF | 23 |
| 363 | CASSLQGEDTQYF | 23 |
| 364 | CASSLAGGEQYF | 23 |
| 365 | CASSLGTGQDTQYF | 23 |
| 366 | CASSTGEDTQYF | 23 |
| 367 | CASSPGTEDTQYF | 23 |
| 368 | CASSDWGYEQYF | 23 |
| 369 | CASSRDRDTQYF | 23 |
| 370 | CASSQGYEQYF | 23 |
| 371 | CASSDRYEQYF | 23 |
| 372 | CASSYYEQYF | 23 |
| 373 | CASSGQGYEQYF | 23 |
| 374 | CASSQEGDTQYF | 23 |
| 375 | CASSQDWEDTQYF | 23 |
| 376 | CASSQDWGSYEQYF | 23 |
| 377 | CASGDVDTQYF | 23 |
| 378 | CASSLGQGDTQYF | 22 |
| 379 | CASSDDEQYF | 22 |
| 380 | CASSLTGGSYEQYF | 22 |
| 381 | CASSLGSDYTF | 22 |
| 382 | CASSQGAEQFF | 22 |
| 383 | CASSSGDTQYF | 22 |
| 384 | CASSLDRGAEQFF | 22 |
| 385 | CASSSGGYEQYF | 22 |
| 386 | CASSWDNYEQYF | 22 |
| 387 | CASSFGDEQYF | 22 |
| 388 | CASSRTGQDTQYF | 22 |
| 389 | CASSLTGQDTQYF | 22 |
| 390 | CASSPGGYEQYF | 22 |
| 391 | CASSPGQGDTQYF | 22 |
| 392 | CASSLPGGYEQYF | 22 |
| 393 | CASSAQDTQYF | 22 |
| 394 | CASSPTGGYEQYF | 22 |
| 395 | CASSLTGSSYEQYF | 22 |
| 396 | CASSLDRDTQYF | 22 |
| 397 | CASSLGGGEQYF | 22 |
| 398 | CASSSDRYEQYF | 22 |
| 399 | CASSLDSEQYF | 22 |
| 400 | CASSLAGDTQYF | 22 |
| 401 | CASSLVGAEQFP | 22 |
| 402 | CASSQDAEQFF | 22 |
| 403 | CASSPTGQDTQYF | 22 |
| 404 | CASSLSGGSYEQYF | 22 |
| 405 | CASSDGYEQYF | 22 |
| 406 | CASSPGLGYEQYF | 22 |
| 407 | CASSRQGEDTQYF | 22 |
| 408 | CASSLLGSSYEQYF | 22 |
| 409 | CASSLGTQDTQYF | 22 |
| 410 | CASSLSGGYAEQFF | 22 |
| 411 | CASSLQGSYEQYF | 22 |
| 412 | CASSLTDTQYF | 22 |
| 413 | CASSFSSYEQYF | 22 |
| 414 | CASSPDRGEQYF | 22 |
| 415 | CASSLGGGQDTQYF | 22 |
| 416 | CASGDIYEQYF | 22 |
| 417 | CASSRDTYEQYF | 22 |
| 418 | CASSSQGDTQYF | 22 |
| 419 | CSADQDTQYF | 22 |
| 420 | CASSPLGYEQYF | 22 |
| 421 | CASSLRDNYEQYF | 22 |
| 422 | CASSLAYEQYF | 22 |
| 423 | CASSLGQSYEQYF | 22 |
| 424 | CASSLVDTQYF | 22 |
| 425 | CASGEGDTQYF | 22 |
| 426 | CASSRTGVYEQYF | 22 |
| 427 | CASSQDRDEQYF | 22 |
| 428 | CASSEGYEQYF | 22 |
| 429 | CASGESSYEQYF | 22 |
| 430 | CASSTGNQDTQYF | 22 |
| 431 | CASSLNSYEQYF | 22 |
| 432 | CASSYAEQFF | 22 |
| 433 | CASSLGTGDTQYF | 22 |
| 434 | CSAGQYEQYF | 22 |
| 435 | CASSQTGYEQYF | 22 |
| 436 | CASSLGLGEDTQYF | 22 |
| 437 | CASSQDRYEQYF | 22 |
| 438 | CASSFGETLYF | 22 |
| 439 | CASSLGTGYEQYF | 22 |

Each possibility represents a separate embodiment of the present invention.

According to some embodiments of the present invention, the CDR3 sequence is selected from the group consisting of the sequences in Table 6.

According to some embodiments of the present invention, the peptide amino acid sequence is selected from the group consisting of SEQ ID NOs of Table 6.

TABLE 6

| | | sharing |
|---|---|---|
| SEQ | | level |
| ID | | in |
| NO: | Sequence | mice |
| 440 | CASGAFNQAPLF | 28 |
| 441 | CASGDAEQFF | 28 |
| 442 | CASGDAGGQDTQYF | 28 |
| 443 | CASGDAGNTLYF | 28 |
| 444 | CASGDAGQNTLYF | 28 |
| 445 | CASGDAGSQNTLYF | 28 |
| 446 | CASGDAGYEQYF | 28 |
| 447 | CASGDGSQNTLYF | 28 |
| 448 | CASGDNYAEQFF | 28 |
| 449 | CASGDQDTQYF | 28 |
| 450 | CASGDRDTQYF | 28 |
| 451 | CASGDSAETLYF | 28 |
| 452 | CASGDSGNTLYF | 28 |
| 453 | CASGDSSQNTLYF | 28 |
| 454 | CASGDWGSAETLYF | 28 |
| 455 | CASGDWGSQNTLYF | 28 |
| 456 | CASRDSAETLYF | 28 |
| 457 | CASRPGTANTGQLYF | 28 |
| 458 | CASSAETLYF | 28 |
| 459 | CASSDRGQNTLYF | 28 |
| 460 | CASSDSAETLYF | 28 |
| 461 | CASSDSQNTLYF | 28 |
| 462 | CASSDSSAETLYF | 28 |
| 463 | CASSDSSQNTLYF | 28 |
| 464 | CASSFDSQNTLYF | 28 |
| 465 | CASSFGQNTLYF | 28 |
| 466 | CASSFGSQNTLYF | 28 |
| 467 | CASSFQANTEVFF | 28 |
| 468 | CASSFSAETLYF | 28 |
| 469 | CASSFSQNTLYF | 28 |
| 470 | CASSGTANSDYTF | 28 |
| 471 | CASSGTTNSDYTF | 28 |
| 472 | CASSHSAETLYF | 28 |
| 473 | CASSHSQNTLYF | 28 |
| 474 | CASSLAANSDYTF | 28 |
| 475 | CASSLAGNYAEQFF | 28 |
| 476 | CASSLAGSQNTLYF | 28 |
| 477 | CASSLANSDYTF | 28 |
| 478 | CASSLANTGQLYF | 28 |
| 479 | CASSLASAETLYF | 28 |
| 480 | CASSLDERLFF | 28 |
| 481 | CASSLDGSQNTLYF | 28 |
| 482 | CASSLDNQDTQYF | 28 |
| 483 | CASSLDNSGNTLYF | 28 |
| 484 | CASSLDNYAEQFF | 28 |
| 485 | CASSLDRYAEQFF | 28 |
| 486 | CASSLDSAETLYF | 28 |
| 487 | CASSLDSDYTF | 28 |
| 488 | CASSLDSQNTLYF | 28 |
| 489 | CASSLDSSAETLYF | 28 |
| 490 | CASSLDSSGNTLYF | 28 |
| 491 | CASSLDSSQNTLYF | 28 |
| 492 | CASSLDSYAEQFF | 28 |
| 493 | CASSLDTEVFF | 28 |
| 494 | CASSLDTGQLYF | 28 |
| 495 | CASSLDWGNYAEQFF | 28 |
| 496 | CASSLDWGQDTQYF | 28 |
| 497 | CASSLDWGSAETLYF | 28 |
| 498 | CASSLEANSDYTF | 28 |
| 499 | CASSLEDSGNTLYF | 28 |
| 500 | CASSLEGAETLYF | 28 |
| 501 | CASSLEGNSDYTF | 28 |
| 502 | CASSLEGNTEVFF | 28 |
| 503 | CASSLEGNTGQLYF | 28 |
| 504 | CASSLEGNTLYF | 28 |
| 505 | CASSLEGNYAEQFF | 28 |
| 506 | CASSLEGSGNTLYF | 28 |
| 507 | CASSLEGSQNTLYF | 28 |
| 508 | CASSLEGYAEQFF | 28 |
| 509 | CASSLESANSDYTF | 28 |
| 510 | CASSLGAETLYF | 28 |
| 511 | CASSLGANSDYTF | 28 |
| 512 | CASSLGDQDTQYF | 28 |
| 513 | CASSLGENTLYF | 28 |
| 514 | CASSLGERLFF | 28 |
| 515 | CASSLGETLYF | 28 |
| 516 | CASSLGGAETLYF | 28 |
| 517 | CASSLGGNSDYTF | 28 |
| 518 | CASSLGGNTEVFF | 28 |
| 519 | CASSLGGNTLYF | 28 |
| 520 | CASSLGGSAETLYF | 28 |
| 521 | CASSLGGSQNTLYF | 28 |
| 522 | CASSLGNQDTQYF | 28 |
| 523 | CASSLGNSGNTLYF | 28 |
| 524 | CASSLENTEVFF | 28 |
| 525 | CASSLGNTGQLYF | 28 |
| 526 | CASSLGNTLYF | 28 |
| 527 | CASSLGQNSDYTF | 28 |
| 528 | CASSLGQNTLYF | 28 |
| 529 | CASSLGQNYAEQFF | 28 |
| 530 | CASSLGQQNTLYF | 28 |
| 531 | CASSLGQSQNTLYF | 28 |
| 532 | CASSLGQTEVFF | 28 |
| 533 | CASSLGSAETLYF | 28 |
| 534 | CASSLGSQNTLYF | 28 |
| 535 | CASSLGSSAETLYF | 28 |
| 536 | CASSLGTANSDYTF | 28 |
| 537 | CASSLGTTNSDYTF | 28 |
| 538 | CASSLLGNYAEQFF | 28 |
| 539 | CASSLNQDTQYF | 28 |
| 540 | CASSLNSAETLYF | 28 |
| 541 | CASSLNIGQLYF | 28 |
| 542 | CASSLQANSDYTF | 28 |
| 543 | CASSLQANTEVFF | 28 |
| 544 | CASSLQGAETLYF | 28 |
| 545 | CASSLQGAGNTLYF | 28 |
| 546 | CASSLQGANTEVFF | 28 |
| 547 | CASSLQGNSDYTF | 28 |
| 548 | CASSLQGNTEVFF | 28 |
| 549 | CASSLQGNTGQLYF | 28 |
| 550 | CASSLQGSAETLYF | 28 |
| 551 | CASSLQGSGNTLYF | 28 |
| 552 | CASSLQGSQNTLYF | 28 |
| 553 | CASSLQGTEVFF | 28 |
| 554 | CASSLQNTLYF | 28 |
| 555 | CASSLRGSQNTLYF | 28 |
| 556 | CASSLSAETLYF | 28 |
| 557 | CASSLSGNTLYF | 28 |
| 558 | CASSLSGSNYAEQFF | 28 |
| 559 | CASSLSQNTLYF | 28 |
| 560 | CASSLSQQNTLYF | 28 |
| 561 | CASSLSSQNTLYF | 28 |
| 562 | CASSLTANSDYTF | 28 |
| 563 | CASSLTANTEVFF | 28 |
| 564 | CASSLTDYNSPLYF | 28 |
| 565 | CASSLTGGYAEQFF | 28 |
| 566 | CASSLTGNYAEQFF | 28 |
| 567 | CASSLTGSQNTLYF | 28 |
| 568 | CASSLTSAETLYF | 28 |
| 569 | CASSLWGSQNTLYF | 28 |
| 570 | CASSPDSAETLYF | 28 |
| 571 | CASSPDSSGNTLYF | 28 |
| 572 | CASSPDSSQNTLYF | 28 |
| 573 | CASSPDWGENTLYF | 28 |
| 574 | CASSPDWGNYAEQFF | 28 |
| 575 | CASSPGDQDTQYF | 28 |
| 576 | CASSPGGQNTLYF | 28 |
| 577 | CASSPGHERLFF | 28 |
| 578 | CASSPGQGNSDYTF | 28 |
| 579 | CASSPGQGYAEQFF | 28 |
| 580 | CASSPGQNTEVFF | 28 |
| 581 | CASSPGQNYAEQFF | 28 |
| 582 | CASSPGQSQNTLYF | 28 |
| 583 | CASSPGSQNTLYF | 28 |
| 584 | CASSPGTANTEVFF | 28 |
| 585 | CASSPNTGQLYF | 28 |
| 586 | CASSPNYAEQFF | 28 |
| 587 | CASSPQGNTEVFF | 28 |
| 588 | CASSPSAETLYF | 28 |
| 589 | CASSPSQNTLYF | 28 |
| 590 | CASSPSSAETLYF | 28 |
| 591 | CASSPTANSDYTF | 28 |
| 592 | CASSPTASQNTLYF | 28 |
| 593 | CASSPTGAETLYF | 28 |
| 594 | CASSPTGSQNTLYF | 28 |
| 595 | CASSPYAEQFF | 28 |
| 596 | CASSQDTEVFF | 28 |
| 597 | CASSQDWGQDTQYF | 28 |
| 598 | CASSQEASNSDYTF | 28 |
| 599 | CASSQGLGDTLYF | 28 |
| 600 | CASSQGQNTEVFF | 28 |
| 601 | CASSQGSQNTLYF | 28 |
| 602 | CASSQNTGQLYF | 28 |
| 603 | CASSQQGSQNTLYF | 28 |
| 604 | CASSQSLDNQDTQYF | 28 |
| 605 | CASSRDIQDTQYF | 28 |
| 606 | CASSRDIYAEQFF | 28 |
| 607 | CASSRDNNERLFF | 28 |
| 608 | CASSRDNQDTQYF | 28 |
| 609 | CASSRDNYAEQFF | 28 |
| 610 | CASSRDRDTEVFF | 28 |
| 611 | CASSRDSAETLYF | 28 |
| 612 | CASSRDSQNTLYF | 28 |
| 613 | CASSRDSSAETLYF | 28 |
| 614 | CASSRDSYAEQFF | 28 |
| 615 | CASSRDWEQNTLYF | 28 |
| 616 | CASSRDWGNYAEQFF | 28 |
| 617 | CASSRDWGQDTQYF | 28 |
| 618 | CASSRGQNTLYF | 28 |
| 619 | CASSRGSAETLYF | 28 |
| 620 | CASSRQANTEVFF | 28 |
| 621 | CASSRQGANSDYTF | 28 |
| 622 | CASSRQGNSDYTF | 28 |
| 623 | CASSRQGNTEVFF | 28 |
| 624 | CASSRQGNTGQLYF | 28 |
| 625 | CASSRQGNYAEQFF | 28 |
| 626 | CASSRQGSQNTLYF | 28 |
| 627 | CASSRQNSDYTF | 28 |
| 628 | CASSRQNTEVFF | 28 |
| 629 | CASSRSQNTLYF | 28 |
| 630 | CASSRVGSDYTF | 28 |
| 631 | CASSSAETLYF | 28 |
| 632 | CASSSDSAETLYF | 28 |
| 633 | CASSSDWGNYAEQFF | 28 |
| 634 | CASSSGDQDTQYF | 28 |
| 635 | CASSSGQNTEVFF | 28 |
| 636 | CASSSGSQNTLYF | 28 |
| 637 | CASSSGTANTEVFF | 28 |
| 638 | CASSSNTGQLYF | 28 |
| 639 | CASSSQGAETLYF | 28 |
| 640 | CASSSQNTLYF | 28 |
| 641 | CASSSSAETLYF | 28 |
| 642 | CASSSSQNTLYF | 28 |
| 643 | CASSTSAETLYF | 28 |
| 644 | CASSWDNYAEQFF | 28 |
| 645 | CASSWDSAETLYF | 28 |
| 646 | CASSWGQNTLYF | 28 |
| 647 | CASSWGSAETLYF | 28 |
| 648 | CASSYSAETLYF | 28 |
| 649 | CASSYSQNTLYF | 28 |
| 650 | CAWSLQGYNSPLYF | 28 |
| 651 | CASGDAGAETLYF | 27 |
| 652 | CASGDAGDTQYF | 27 |
| 653 | CASGDAGGQNTLYF | 27 |
| 654 | CASGDAGGYAEQFF | 27 |
| 655 | CASGDAGNTGQLYF | 27 |
| 656 | CASGDAGQNTEVFF | 27 |
| 657 | CASGDAGSAETLYF | 27 |
| 658 | CASGDANSDYTF | 27 |
| 659 | CASGDARDTQYF | 27 |
| 660 | CASGDASAETLYF | 27 |
| 661 | CASGDNTEVFF | 27 |
| 662 | CASGDNYNSPLYF | 27 |
| 663 | CASGDSSAETLYF | 27 |
| 664 | CASGDWGNQDTQYF | 27 |
| 665 | CASGDWGNYAEQFF | 27 |
| 666 | CASGENTLYF | 27 |
| 667 | CASGGQNYAEQFF | 27 |
| 668 | CASGGSQNTLYF | 27 |
| 669 | CASGQNTLYF | 27 |
| 670 | CASKTANQDTQYF | 27 |
| 671 | CASRDNSGNTLYF | 27 |
| 672 | CASRDSSQNTLYF | 27 |
| 673 | CASRRDSAETLYF | 27 |
| 674 | CASRTGGYAEQFF | 27 |
| 675 | CASRTSAETLYF | 27 |
| 676 | CASSASAETLYF | 27 |
| 677 | CASSDAGQNTLYF | 27 |
| 678 | CASSDANSDYTF | 27 |
| 679 | CASSDGSQNTLYF | 27 |
| 680 | CASSDNQDTQYF | 27 |
| 681 | CASSDNYAEQFF | 27 |
| 682 | CASSDSSGNTLYF | 27 |
| 683 | CASSDWGSAETLYF | 27 |
| 684 | CASSESQNTLYF | 27 |
| 685 | CASSFDSAETLYF | 27 |
| 686 | CASSEDTEVFF | 27 |
| 687 | CASSTGGQNTLYF | 27 |
| 688 | CASSFQNTLYF | 27 |
| 689 | CASSFSSAETLYF | 27 |
| 690 | CASSGAETLYF | 27 |
| 691 | CASSGQANTEVFF | 27 |
| 692 | CASSGQNYAEQFF | 27 |
| 693 | CASSGTASAETLYF | 27 |
| 694 | CASSGTGGYAEQFF | 27 |
| 695 | CASSGTISNERLFF | 27 |
| 696 | CASSGTVSNERLFF | 27 |
| 697 | CASSLAETLYF | 27 |
| 698 | CASSLAGGYAEQFF | 27 |
| 699 | CASSLAGSGNTLYF | 27 |
| 700 | CASSLANSGNTLYF | 27 |
| 701 | CASSLASQNTLYF | 27 |
| 702 | CASSLDAFTLYF | 27 |
| 703 | CASSLDANSDYTF | 27 |
| 704 | CASSLDNSDYTF | 27 |
| 705 | CASSLDNSQNTLYF | 27 |
| 706 | CASSLDNTEVFF | 27 |
| 707 | CASSIDQNTLYF | 27 |
| 708 | CASSLDRDTEVFF | 27 |
| 709 | CASSLDREVFF | 27 |
| 710 | CASSLDRGAETLYF | 27 |
| 711 | CASSLDRNSDYTF | 27 |
| 712 | CASSLDRNTEVFF | 27 |
| 713 | CASSLDRNTGQLYF | 27 |
| 714 | CASSLDTNSDYTF | 27 |
| 715 | CASSLDWGNTGQLYF | 27 |
| 716 | CASSLDWGQNTLYF | 27 |
| 717 | CASSLDWGSQNTLYF | 27 |
| 718 | CASSLDWGYAEQFF | 27 |
| 719 | CASSLDYAEQFF | 27 |
| 720 | CASSLEANTEVFF | 27 |
| 721 | CASSLEGAGNTLYF | 27 |
| 722 | CASSLEGGQNTLYF | 27 |
| 723 | CASSLEGSIDYTF | 27 |
| 724 | CASSLEGTGQLYF | 27 |
| 725 | CASSLENTEVFF | 27 |
| 726 | CASSLENTLYF | 27 |
| 727 | CASSLEQNSDYTF | 27 |
| 728 | CASSLETLYF | 27 |
| 729 | CASSLGANTEVFF | 27 |
| 730 | CASSLGANIGQLYF | 27 |
| 731 | CASSLGDTEVFF | 27 |
| 732 | CASSLGGNTGQLYF | 27 |
| 733 | CASSLGGNYAEQFF | 27 |
| 734 | CASSLGGSGNTLYF | 27 |
| 735 | CASSLGGTEVFF | 27 |
| 736 | CASSLGGYAEQFF | 27 |
| 737 | CASSLGNYAEQFF | 27 |
| 738 | CASSLGQANTEVFF | 27 |
| 739 | CASSLGQGAETLYF | 27 |
| 740 | CASSLGQGNTEVFF | 27 |
| 741 | CASSLGQGYAEQFF | 27 |
| 742 | CASSLGQYAEQFF | 27 |
| 743 | CASSLGTANTEVFF | 27 |
| 744 | CASSLGTEVFF | 27 |
| 745 | CASSLGTNTEVFF | 27 |
| 746 | CASSLGVNQDTQYF | 27 |
| 747 | CASSLGVYAEQFF | 27 |
| 748 | CASSLLGGYAEQFF | 27 |
| 749 | CASSLNSDYTF | 27 |
| 750 | CASSLNSQNTLYF | 27 |
| 751 | CASSLNTEVFF | 27 |
| 752 | CASSLQGANSDYTF | 27 |
| 753 | CASSLQGNTLYF | 27 |
| 754 | CASSLQGNYAEQFF | 27 |
| 755 | CASSLQGQNTLYF | 27 |
| 756 | CASSLQGYAEQFF | 27 |
| 757 | CASSLQNSDYTF | 27 |
| 758 | CASSLQNTEVFF | 27 |
| 759 | CASSLRGQNTLYF | 27 |
| 760 | CASSLRSQNTLYF | 27 |
| 761 | CASSLSGGQNTLYF | 27 |
| 762 | CASSLSSAETLYF | 27 |
| 763 | CASSLTANTGQLYF | 27 |
| 764 | CASSLTGAETLYF | 27 |
| 765 | CASSLTGANTGQLYF | 27 |
| 766 | CASSLTGGQNTLYF | 27 |
| 767 | CASSLTTNTEVFF | 27 |
| 768 | CASSLTTSAETLYF | 27 |
| 769 | CASSLVGNQDTQYF | 27 |
| 770 | CASSLVGSQNTLYF | 27 |
| 771 | CASSNSAETLYF | 27 |
| 772 | CASSPAETLYF | 27 |
| 773 | CASSPDSNERLFF | 27 |
| 774 | CASSPDWGSQNTLYF | 27 |
| 775 | CASSPGAGSNERLFF | 27 |
| 776 | CASSPGLGNYAEQFF | 27 |
| 777 | CASSPGLGQDTQYF | 27 |
| 778 | CASSPGNTEVFF | 27 |
| 779 | CASSPGQGAETLYF | 27 |
| 780 | CASSPGQNTGQLYF | 27 |
| 781 | CASSPGSAETLYF | 27 |
| 782 | CASSPGTANTGQLYF | 27 |
| 783 | CASSPGTTNSDYTF | 27 |
| 784 | CASSPQGAETLYF | 27 |
| 785 | CASSPQGSAETLYF | 27 |
| 786 | CASSPTANTEVFF | 27 |
| 787 | CASSPTGGQNTLYF | 27 |
| 788 | CASSQDSAETLYF | 27 |
| 789 | CASSQDSSGNTLYF | 27 |
| 790 | CASSQDSSQNTLYF | 27 |
| 791 | CASSQDWGNYAEQFF | 27 |
| 792 | CASSQGAGNTLYF | 27 |
| 793 | CASSQNTLYF | 27 |
| 794 | CASSQQGNTEVFF | 27 |
| 795 | CASSQTSAETLYF | 27 |
| 796 | CASSRAETLYF | 27 |
| 797 | CASSRDKNTLYF | 27 |
| 798 | CASSRDNQNTLYF | 27 |
| 799 | CASSRDNSDYTF | 27 |
| 800 | CASSRDNSQNTLYF | 27 |
| 801 | CASSRDRDAEQFF | 27 |
| 802 | CASSRDREVFF | 27 |
| 803 | CASSRDRGNTLYF | 27 |
| 804 | CASSRDRNITEVFF | 27 |
| 805 | CASSRDSNERLFF | 27 |
| 806 | CASSRDSNYAEQFF | 27 |
| 807 | CASSRDSSQNTLYF | 27 |
| 808 | CASSRDWGNTLYF | 27 |
| 809 | CASSRDWGSQNTLYF | 27 |
| 810 | CASSRDWGYAEQFF | 27 |
| 811 | CASSRGSQNTLYF | 27 |
| 812 | CASSRLGNYAEQFF | 27 |
| 813 | CASSRQGAETLYF | 27 |
| 814 | CASSRQGNTLYF | 27 |
| 815 | CASSRQGQNTLYF | 27 |
| 816 | CASSRQNYAEQFF | 27 |
| 817 | CASSRTGENTLYF | 27 |
| 818 | CASSRTGQNTLYF | 27 |
| 819 | CASSSGGQNTLYF | 27 |
| 820 | CASSSGNTLYF | 27 |
| 821 | CASSSGNYAEQFF | 27 |
| 822 | CASSSGQQNTLYF | 27 |
| 823 | CASSSGTTNTEVFF | 27 |
| 824 | CASSSQGANTEVFF | 27 |
| 825 | CASSSTANSDYTF | 27 |
| 826 | CASSWGSQNTLYF | 27 |
| 827 | CASSYGQNTLYF | 27 |
| 828 | CGARDSAETLYF | 27 |
| 829 | CSAGGQNTLYF | 27 |
| 830 | CASGAETLYF | 26 |
| 831 | CASGDADSGNTLYF | 26 |
| 832 | CASGDAETLYF | 26 |
| 833 | CASGDAGDQDTQYF | 26 |
| 834 | CASGDAGEQYF | 26 |
| 835 | CASGDAGGNTLYF | 26 |
| 836 | CASGDAGNSDYTF | 26 |
| 837 | CASGDANSGNTLYF | 26 |
| 838 | CASGDASQNTLYF | 26 |
| 839 | CASGDAYAEQFF | 26 |
| 840 | CASGDNSGNTLYF | 26 |
| 841 | CASGDPGNYAEQFF | 26 |
| 842 | CASGDRGNTEVFF | 26 |
| 843 | CASGDRYEQYF | 26 |
| 844 | CASGDSQNTLYF | 26 |
| 845 | CASGDSSGNTLYF | 26 |
| 846 | CASGDSYAEQFF | 26 |
| 847 | CASGDYAEQFF | 26 |
| 848 | CASGEGQNTLYF | 26 |
| 849 | CASGESAETLYF | 26 |
| 850 | CASGGQANTEVFF | 26 |
| 851 | CASRDWGNYAEQFF | 26 |
| 852 | CASRGAETLYF | 26 |
| 853 | CASSASQNTLYF | 26 |
| 854 | CASSDAGNTLYF | 26 |
| 855 | CASSDAGSQNTLYF | 26 |
| 856 | CASSDASAETLYF | 26 |
| 857 | CASSDRAETLYF | 26 |
| 858 | CASSDRDTEVFF | 26 |
| 859 | CASSMINTEVFF | 26 |
| 860 | CASSDWDQDTQYF | 26 |
| 861 | CASSDWGNYAEQFF | 26 |
| 862 | CASSDWGQNTLYF | 26 |
| 863 | CASSFGAETLYF | 26 |
| 864 | CASSFGGAETLYF | 26 |
| 865 | CASSFGSAETLYF | 26 |
| 866 | CASSFPSGNTLYF | 26 |
| 867 | CASSFQGNTEVFF | 26 |
| 868 | CASSFSGAQDTQYF | 26 |
| 869 | CASSGDSQNTLYF | 26 |
| 870 | CASSGDSYAEQFF | 26 |
| 871 | CASSGLGNYAEQFF | 26 |
| 872 | CASSGQGAETLYF | 26 |
| 873 | CASSGTANTGQLYF | 26 |
| 874 | CASSGTSNSDYTF | 26 |
| 875 | CASSLAANTGQLYF | 26 |
| 876 | CASSLAGDTGQLYF | 26 |
| 877 | CASSLAGNQDTQYF | 26 |
| 878 | CASSLAGNTEVFF | 26 |
| 879 | CASSLAGNTGQLYF | 26 |
| 880 | CASSLAGSAETLYF | 26 |
| 881 | CASSEDANTEVFF | 26 |
| 882 | CASSLDGNTLYF | 26 |
| 883 | CASSLDIYAEQFF | 26 |
| 884 | CASSLDNERLFF | 26 |
| 885 | CASSLDNNQDTQYF | 26 |
| 886 | CASSLDQAPLF | 26 |
| 887 | CASSLDRNTLYF | 26 |
| 888 | CASSLDSGNTLYF | 26 |
| 889 | CASSLDSNQDTQYF | 26 |
| 890 | CASSLDSQDTQYF | 26 |
| 891 | CASSLDTSQNTLYF | 26 |
| 892 | CASSLDWGNTLYF | 26 |
| 893 | CASSUANTGQLYF | 26 |
| 894 | CASSLEDTGQLYF | 26 |
| 895 | CASSLEGANSDYTF | 26 |
| 896 | CASSLEGQNTLYF | 26 |
| 897 | CASSLEGSAETLYF | 26 |
| 898 | CASSLENTGQLYF | 26 |
| 899 | CASSLESAETLYF | 26 |
| 900 | CASSLGASQNTLYF | 26 |
| 901 | CASSLGDNYAEQFF | 26 |
| 902 | CASSLGDSAETLYF | 26 |
| 903 | CASSLGDSGNTLYF | 26 |
| 904 | CASSLGDTGQLYF | 26 |
| 905 | CASSLGGSNERLFF | 26 |
| 906 | CASSLGQNTGQLYF | 26 |
| 907 | CASSLGQSAETLYF | 26 |
| 908 | CASSLGSGNTLYF | 26 |
| 909 | CASSLGTGQLYF | 26 |
| 910 | CASSLGTSAETLYF | 26 |
| 911 | CASSLGTTNTEVFF | 26 |
| 912 | CASSIGVSNERLFF | 26 |
| 913 | CASSLLGDQDTQYF | 26 |
| 914 | CASSLQGGNTLYF | 26 |
| 915 | CASSLQGNQDTQYF | 26 |
| 916 | CASSLQISNERLFF | 26 |
| 917 | CASSLQSQNTLYF | 26 |
| 918 | CASSLRAETLYF | 26 |
| 919 | CASSLRDNQDTQYF | 26 |
| 920 | CASSLRDTGQLYF | 26 |
| 921 | CASSLRGNTEVFF | 26 |
| 922 | CASSLRGSGNTLYF | 26 |
| 923 | CASSLSGSAETLYF | 26 |
| 924 | CASSLSNERLFF | 26 |
| 925 | CASSLSTGQLYF | 26 |
| 926 | CASSLTASQNTLYF | 26 |
| 927 | CASSLTDSGNTLYF | 26 |
| 928 | CASSLTENTLYF | 26 |
| 929 | CASSLTGANTEVFF | 26 |
| 930 | CASSLTGDQDTQYF | 26 |
| 931 | CASSLTGGNTLYF | 26 |
| 932 | CASSLTGGSQNTLYF | 26 |
| 933 | CASSLTGNTLYF | 26 |
| 934 | CASSLTGQNTLYF | 26 |
| 935 | CASSLIGSAETLYF | 26 |
| 936 | CASSLTNTEVFF | 26 |
| 937 | CASSLTSSAETLYF | 26 |
| 938 | CASSLVGNYAEQFF | 26 |
| 939 | CASSLVNQDTQYF | 26 |
| 940 | CASSINSQNTLYF | 26 |
| 941 | CASSLWGNYAEQFF | 26 |
| 942 | CASSPDNSGNTLYF | 26 |
| 943 | CASSPDSQNTLYF | 26 |
| 944 | CASSPDWGQNTLYF | 26 |
| 945 | CASSPGAETLYF | 26 |
| 946 | CASSPGGNYAEQFF | 26 |
| 947 | CASSTGQYAEQFF | 26 |
| 948 | CASSPGQYNSPLYF | 26 |
| 949 | CASSPGTSSAETLYF | 26 |
| 950 | CASSPGTTNTEVFF | 26 |
| 951 | CASSPGTTSAETLYF | 26 |
| 952 | CASSPQANTGQLYF | 26 |
| 953 | CASSPQGAGNTLYF | 26 |
| 954 | CASSPQGNTGQLYF | 26 |
| 955 | CASSPQGSQNTLYF | 26 |
| 956 | CASSPTANTGQLYF | 26 |
| 957 | CASSPTGGNYAEQFF | 26 |
| 958 | CASSPTGNTGQLYF | 26 |
| 959 | CASSPTGNYAEQFF | 26 |
| 960 | CASSPTSAETLYF | 26 |
| 961 | CASSPTSSQNTLYF | 26 |
| 962 | CASSQDSQNTLYF | 26 |
| 963 | CASSQDWGSQNTLYF | 26 |
| 964 | CASSQGNYAEQFF | 26 |
| 965 | CASSQGSAETLYF | 26 |
| 966 | CASSQGSGNTLYF | 26 |
| 967 | CASSQGTANTGQLYF | 26 |
| 968 | CASSQNTEVFF | 26 |
| 969 | CASSQQGANTEVFF | 26 |
| 970 | CASSRDNSAETLYF | 26 |
| 971 | CASSRDNSGNTLYF | 26 |
| 972 | CASSRDRGAEQFF | 26 |
| 973 | CASSRDRNTGQLYF | 26 |
| 974 | CASSRDSSGNTLYF | 26 |
| 975 | CASSRDTGQLYF | 26 |
| 976 | CASSRDWGNTGQLYF | 26 |
| 977 | CASSRLGQDTQYF | 26 |
| 978 | CASSRNTGQLYF | 26 |
| 979 | CASSRQANSDYTF | 26 |
| 980 | CASSRQGANTEVFF | 26 |
| 981 | CASSRQGANTGQLYF | 26 |
| 982 | CASSRQNTGQLYF | 26 |
| 983 | CASSRTDSGNTLYF | 26 |
| 984 | CASSRTSQNTLYF | 26 |
| 985 | CASSSDSQNTLYF | 26 |
| 986 | CASSSDWGQDTQYF | 26 |
| 987 | CASSSGNTEVFF | 26 |
| 988 | CASSSGQANTEVFF | 26 |
| 989 | CASSSGQNTLYF | 26 |
| 990 | CASSSGQQDTQYF | 26 |
| 991 | CASSSGTNTEVFF | 26 |
| 992 | CASSSQGNSDYTF | 26 |
| 993 | CASSSQGNIEVFF | 26 |
| 994 | CASSSQGNTGQLYF | 26 |
| 995 | CASSSSGNTLYF | 26 |
| 996 | CASSSTANTEVFF | 26 |
| 997 | CASSSTASQNTLYF | 26 |
| 998 | CASSSTGNTEVFF | 26 |
| 999 | CASSSTSAETLYF | 26 |
| 1000 | CASSSYAEQFF | 26 |
| 1001 | CASSTGNTGQLYF | 26 |
| 1002 | CASSWIDSQNTLYF | 26 |
| 1003 | CASSWDSYAEQFF | 26 |
| 1004 | CASSWGNYAEQFF | 26 |
| 1005 | CSKDSAETLYF | 26 |
| 1006 | CSSSQGTNERLFF | 26 |
| 1007 | CASGDADEQYF | 25 |
| 1008 | CASGDADTGQLYF | 25 |
| 1009 | CASGDAGAEQFF | 25 |
| 1010 | CASGDAGANSDYTF | 25 |
| 1011 | CASGDAGDTGQLYF | 25 |
| 1012 | CASGDAGGAETLYF | 25 |
| 1013 | CASGDAGNQDTQYF | 25 |
| 1014 | CASGDAGNTEVFF | 25 |
| 1015 | CASGDAGNYAEQFF | 25 |
| 1016 | CASGDAGQDTQYF | 25 |
| 1017 | CASGDAQSGNTLYF | 25 |
| 1018 | CASGDDQDTQYF | 25 |
| 1019 | CASGDGGQNTLYF | 25 |
| 1020 | CASGDNSQNTLYF | 25 |
| 1021 | CASGDPSAETLYF | 25 |
| 1022 | CASGDPSQNTLYF | 25 |
| 1023 | CASGDRGSGNTLYF | 25 |
| 1024 | CASGDRGSQNTLYF | 25 |
| 1025 | CASGDRNTEVFF | 25 |
| 1026 | CASGDSNERLFF | 25 |
| 1027 | CASGEGGQNTLYF | 25 |
| 1028 | CASGGQGNTEVFF | 25 |
| 1029 | CASGGTANTEVFF | 25 |
| 1030 | CASGSAETLYF | 25 |
| 1031 | CASRDNYAEQFF | 25 |
| 1032 | CASRDSNYAEQFF | 25 |
| 1033 | CASRDSSGNTLYF | 25 |
| 1034 | CASRDWGSAETLYF | 25 |
| 1035 | CASRGQNYAEQFF | 25 |
| 1036 | CASSDAGGQNTLYF | 25 |
| 1037 | CASSDANTEVFF | 25 |
| 1038 | CASSDGAETLYF | 25 |
| 1039 | CASSDGGNYAEQFF | 25 |
| 1040 | CASSDNSGNTLYF | 25 |
| 1041 | CASSDNSQNTLYF | 25 |
| 1042 | CASSDNTEVFF | 25 |
| 1043 | CASSDRGAETLYF | 25 |
| 1044 | CASSDRNSDYTF | 25 |
| 1045 | CASSEGQNTLYF | 25 |
| 1046 | CASSEGSQNTLYF | 25 |
| 1047 | CASSENSGNTLYF | 25 |
| 1048 | CASSENTGQLYF | 25 |
| 1049 | CASSETANTEVFF | 25 |
| 1050 | CASSFGGNYAEQFF | 25 |
| 1051 | CASSFGGSAFTLYF | 25 |
| 1052 | CASSFGTANTEVFF | 25 |
| 1053 | CASSFNYAEQFF | 25 |
| 1054 | CASSGQGNTEVFF | 25 |
| 1055 | CASSGQGQNTLYF | 25 |
| 1056 | CASSGSAETLYF | 25 |
| 1057 | CASSGSQNTLYF | 25 |
| 1058 | CASSGTGNSDYTF | 25 |
| 1059 | CASSGTGQNTLYF | 25 |
| 1060 | CASSGTTNTGQLYF | 25 |
| 1061 | CASSNQDTQYF | 25 |
| 1062 | GASSLAANTEVFF | 25 |
| 1063 | CASSLAGAETLYF | 25 |
| 1064 | CASSLAGDQDTQYF | 25 |
| 1065 | CASSLAGENTLYF | 25 |
| 1066 | CASSLAGQNTLYF | 25 |
| 1067 | CASSLDGGQNTLYF | 25 |
| 1068 | CASSLDGNTEVFF | 25 |
| 1069 | CASSLDGYAEQFF | 25 |
| 1070 | CASSLDNTGQLYF | 25 |
| 1071 | CASSLDRAGNTLYF | 25 |
| 1072 | CASSLDRANSDYTF | 25 |
| 1073 | CASSLDRDAEQFF | 25 |
| 1074 | CASSLDRGEVFF | 25 |
| 1075 | CASSLDRTEVFF | 25 |
| 1076 | CASSLEGANTEVFF | 25 |
| 1077 | GASSLEGDQDTQYF | 25 |
| 1078 | CASSLEGDSDYTF | 25 |
| 1079 | CASSLEGDTGQLYF | 25 |
| 1080 | CASSLEGSSAETLYF | 25 |
| 1081 | CASSLEGTSAETLYF | 25 |
| 1082 | CASSLENSDYTF | 25 |
| 1083 | CASSLESQNTLYF | 25 |
| 1084 | CASSLGGGQNTLYF | 25 |
| 1085 | CASSLGGGYAEQFF | 25 |
| 1086 | CASSLGGSDYTF | 25 |
| 1087 | CASSLGHQDTQYF | 25 |
| 1088 | CASSLGISNERLFF | 25 |
| 1089 | CASSLGLGAETLYF | 25 |
| 1090 | CASSLGLGYAEQFF | 25 |
| 1091 | CASSLGNSAETLYF | 25 |
| 1092 | CASLGQGTEVFF | 25 |
| 1093 | CASSLGSSQNTLYF | 25 |
| 1094 | CASSLGTANTGQLYF | 25 |
| 1095 | CASSLGTASAETLYF | 25 |
| 1096 | CASSLGTSSAETLYF | 25 |
| 1097 | CASSLGVNYAEQFF | 25 |
| 1098 | CASSLGVSQNTLYF | 25 |
| 1099 | CASSLLGANTGQLYF | 25 |
| 1100 | CASSLPSAETLYT | 25 |
| 1101 | CASSLQGANTGQLYF | 25 |
| 1102 | CASSLQGDQDTQYF | 25 |
| 1103 | CASSLQGSDYTF | 25 |
| 1104 | CASSLQQDTQYF | 25 |
| 1105 | CASSLRQNTEVFF | 25 |
| 1106 | CASSLRSAETLYF | 25 |
| 1107 | CASSLSGAETLYF | 25 |
| 1108 | CASSLSGNYAEQFF | 25 |
| 1109 | CASSLSGQNTLYF | 25 |
| 1110 | CASSLSGSGNTLYF | 25 |
| 1111 | CASSLSGSQNTLYF | 25 |
| 1112 | CASSLSNTEVFF | 25 |
| 1113 | CASSLTASAETLYF | 25 |
| 1114 | CASSLTGDYAEQFF | 25 |
| 1115 | CASSLTGENTLYF | 25 |
| 1116 | CASSLTGGAETLYF | 25 |
| 1117 | CASSLTGNTGQLYF | 25 |
| 1118 | CASSLTSQNTLYF | 25 |
| 1119 | CASSLVSAETLYF | 25 |
| 1120 | GASSNTGQLYF | 25 |
| 1121 | CASSNYAEQFF | 25 |
| 1122 | CASSPDNYAEQFF | 25 |
| 1123 | CASSPDRNTEVFF | 25 |
| 1124 | CASSPDRSQNTLYF | 25 |
| 1125 | GASSPDWGQDTQYF | 25 |
| 1126 | CASSPGGAUTLYF | 25 |
| 1127 | CASSPGGSQNTLYF | 25 |
| 1128 | CASSPGNSDYTF | 25 |
| 1129 | CASSPGQANTEVFF | 25 |
| 1130 | CASSPGQGQNTLYF | 25 |
| 1131 | CASSPGQGTEVFF | 25 |
| 1132 | CASSPGQNQDTQYF | 25 |
| 1133 | CASSPGQNSDYTF | 25 |
| 1134 | CASSPGQNTLYF | 25 |
| 1135 | CASSPGQQNTLYF | 25 |
| 1136 | CASSPGQIEVFF | 25 |
| 1137 | CASSPGTAETLYF | 25 |
| 1138 | CASSPGTANSDYTF | 25 |
| 1139 | CASSPGTASAETLYF | 25 |
| 1140 | CASSPGTGYAEQFF | 25 |
| 1141 | CASSPGTNTEVFF | 25 |
| 1142 | CASSPGTTNERLFF | 25 |
| 1143 | CASSPLGSQNTLYF | 25 |
| 1144 | CASSPTGGYAEQFF | 25 |
| 1145 | CASSPTGNQDTQYF | 25 |
| 1146 | CASSPTTSQNTLYF | 25 |
| 1147 | CASSPTVSNERLFF | 25 |
| 1148 | CASSPTVSQNTLYF | 25 |
| 1149 | CASSQANTGQLYF | 25 |
| 1150 | CASSQDSYAEQFT | 25 |
| 1151 | CASSQEGSQNTLYF | 25 |
| 1152 | CASSQGAETLYF | 25 |
| 1153 | CASSQGGQNTLYF | 25 |
| 1154 | CASSQGNTGQLYF | 25 |
| 1155 | CASSQGQNTLYF | 25 |
| 1156 | CASSQGQNYAEQFF | 25 |
| 1157 | CASSQNSGNTLYF | 25 |
| 1158 | CASSQQGAETLYF | 25 |
| 1159 | CASSQQGNTGQLYF | 25 |
| 1160 | CASSQQGSGNTLYF | 25 |
| 1161 | CASSRDISQNTLYF | 25 |
| 1162 | CASSRDNNYAEQFF | 25 |
| 1163 | CASSRDNQAPLF | 25 |
| 1164 | CASSRDNTEVFF | 25 |
| 1165 | CASSRDRANSDYTF | 25 |
| 1166 | CASSRDRNTLYF | 25 |
| 1167 | CASSRDRNYAEQFF | 25 |
| 1168 | CASSRDSANSDYTF | 25 |
| 1169 | CASSRDTEVFF | 25 |
| 1170 | CASSRGAETLYF | 25 |
| 1171 | CASSRGNQDTQYF | 25 |
| 1172 | CASSRGNYAEQFF | 25 |
| 1173 | CASSRLGSQNTLYF | 25 |
| 1174 | CASSRNQDTQYF | 25 |
| 1175 | CASSRNTEVFF | 25 |
| 1176 | CASSRQGAGNTLYF | 25 |
| 1177 | CASSRQGDTEVFF | 25 |
| 1178 | CASSRQSQNTLYF | 25 |
| 1179 | CASSRTANSDYTF | 25 |
| 1180 | CASSRTASAETLYF | 25 |
| 1181 | CASSRTGGQNTLYF | 25 |
| 1182 | CASSRTGGYAEQFF | 25 |
| 1183 | CASSRTGNSDYTF | 25 |
| 1184 | CASSRTGNTGQLYF | 25 |
| 1185 | CASSRTGSQNTLYF | 25 |
| 1186 | CASSRTNTEVFF | 25 |
| 1187 | CASSRTTNTEVFF | 25 |
| 1188 | CASSRYAEQFF | 25 |
| 1189 | CASSSAANTEVFF | 25 |
| 1190 | CASSSANIGQLYF | 25 |
| 1191 | GASSSETLYF | 25 |
| 1192 | CASSSGAETLYF | 25 |
| 1193 | CASSSGGAETLYF | 25 |
| 1194 | CASSSGGYAEQFF | 25 |
| 1195 | CASSSGNQDIQYF | 25 |
| 1196 | CASSSGQGAETLYF | 25 |
| 1197 | CASSSGQGNTEVFF | 25 |
| 1198 | CASSSGSAETLYF | 25 |
| 1199 | CASSSGTGNTEVFF | 25 |
| 1200 | CASSSNERLFF | 25 |
| 1201 | CASSSNYAEQFF | 25 |
| 1202 | CASSSQANTEVFF | 25 |
| 1203 | CASSSQGSAETLYF | 25 |
| 1204 | CASSSTGGYAEQFF | 25 |
| 1205 | CASSTSQNTLYF | 25 |
| 1206 | CASSWDNTEVFF | 25 |
| 1207 | CASSWGNQDTQYF | 25 |
| 1208 | CAWSLGSQNTLYF | 25 |
| 1209 | CGARDSNYAEQFF | 25 |
| 1210 | CGARDWGYAEQFF | 25 |
| 1211 | CGARQGYAEQFF | 25 |
| 1212 | CSAGSQNTLYF | 25 |
| 1213 | CSASAETLYF | 25 |
| 1214 | CSASQNTLYF | 25 |
| 1215 | CASGDADQDTQYF | 24 |
| 1216 | CASGDAEQYF | 24 |
| 1217 | CASGDAGGNYAEQFF | 24 |
| 1218 | CASGDAGSSYEQYF | 24 |
| 1219 | CASGDANTEVFF | 24 |
| 1220 | CASGDAPSQNTLYF | 24 |
| 1221 | CASGDDAETLYF | 24 |
| 1222 | CASGDGGNYAEQFF | 24 |
| 1223 | CASGDRANTEVFF | 24 |
| 1224 | CASGDRDQDTQYF | 24 |
| 1225 | CASGDRGAETLYF | 24 |
| 1226 | CASGDRNYAEQFF | 24 |
| 1227 | CASGDTNSDYTF | 24 |
| 1228 | CASGDVEQYF | 24 |
| 1229 | CASGDWDSAETLYF | 24 |
| 1230 | CASGDWGNTGQLYF | 24 |
| 1231 | CASGDWGQNTLYF | 24 |
| 1232 | CASGEGSQNTLYF | 24 |
| 1233 | CASGETDSGNTLYF | 24 |
| 1234 | CASGGTANSDYTF | 24 |
| 1235 | CASGTTNTEVFF | 24 |
| 1236 | CASRDRNYAEQFF | 24 |
| 1237 | CASRNTGQLYF | 24 |
| 1238 | CASRQGSQNTLYF | 24 |
| 1239 | CASRWDNYEQYF | 24 |
| 1240 | CASSDANSGNTLYF | 24 |
| 1241 | CASSDASQNTLYF | 24 |
| 1242 | CASSDDNSGNTLYF | 24 |
| 1243 | CASSDGDTGQLYF | 24 |
| 1244 | CASSDGGQNTLYF | 24 |
| 1245 | CASSDNNERLFF | 24 |
| 1246 | CASSDNNQAPLF | 24 |
| 1247 | CASSDQNTLYF | 24 |
| 1248 | CASSDRGNTEVFF | 24 |
| 1249 | CASSDRGSGNTLYF | 24 |
| 1250 | CASSDRNYAEQFF | 24 |
| 1251 | CASSDSGNTLYF | 24 |
| 1252 | CASSDSNERLFF | 24 |
| 1253 | CASSDWGNQDTQYF | 24 |
| 1254 | CASSDYAEQFF | 24 |
| 1255 | CASSEGAETLYF | 24 |
| 1256 | CASSFAETLYF | 24 |
| 1257 | CASSFDWGNYAEQFF | 24 |
| 1258 | CASSFGGSQNTLYF | 24 |
| 1259 | CASSFNTGQLYF | 24 |
| 1260 | CASSFRAETLYF | 24 |
| 1261 | CASSFRGSQNTLYF | 24 |
| 1262 | CASSFRNTEVFF | 24 |
| 1263 | CASSFSGNTLYF | 24 |
| 1264 | CASSGDNYAEQFF | 24 |
| 1265 | CASSGDSAETLYF | 24 |
| 1266 | CASSGDSSGNTLYF | 24 |
| 1267 | CASSGQGNSDYTF | 24 |
| 1268 | CASSGQSQNTLYF | 24 |
| 1269 | CASSGTAETLYF | 24 |
| 1270 | CASSGTGGAETLYF | 24 |
| 1271 | CASSGTGGNYAEQFF | 24 |
| 1272 | CASSGTGNYAEQFF | 24 |
| 1273 | CASSGTTNTEVFF | 24 |
| 1274 | CASSIGQNTEVFF | 24 |
| 1275 | CASSIRDNYAEQFF | 24 |
| 1276 | CASSISAETLYF | 24 |
| 1277 | CASSLAGANTEVFF | 24 |
| 1278 | CASSLAGNTLYF | 24 |
| 1279 | CASSLANTEVFF | 24 |
| 1280 | CASSLDGAETLYF | 24 |
| 1281 | CASSLDGQNTLYF | 24 |
| 1282 | CASSLDGSSNETLYF | 24 |
| 1283 | CASSLDKNTLYF | 24 |
| 1284 | CASSLDRDSDYTF | 24 |
| 1285 | CASSLDRNYAEQFF | 24 |
| 1286 | CASSLDSNERLFF | 24 |
| 1287 | CASSEDSNYAEQFF | 24 |
| 1288 | CASSLDSYNSPLYF | 24 |
| 1289 | CASSLDTNTEVFF | 24 |
| 1290 | CASSLEGDAEQFF | 24 |
| 1291 | CASSLEGGYAEQFF | 24 |
| 1292 | CASSLEGQQDTQYF | 24 |
| 1293 | CASSLEGTEVFF | 24 |
| 1294 | CASSLENSGNTLYF | 24 |
| 1295 | CASSLENYAEQFF | 24 |
| 1296 | CASSLEQTEVFF | 24 |
| 1297 | CASSEGDYAEQFF | 24 |
| 1298 | CASSLGENTEVFF | 24 |
| 1299 | CASSLGEVFF | 24 |
| 1300 | CASSLGGANTEVFF | 24 |
| 1301 | CASSLGGTGQLYF | 24 |
| 1302 | CASSLGLYAEQFF | 24 |
| 1303 | CASSLGNQAPLF | 24 |
| 1304 | CASSLGQGNSDYTF | 24 |
| 1305 | CASSLGTENTLYF | 24 |
| 1306 | CASSLGTGAETLYF | 24 |
| 1307 | CASSLGTGNTGQLYF | 24 |
| 1308 | CASSLGTNSDYTF | 24 |
| 1309 | CASSLGTSQNTLYF | 24 |
| 1310 | CASSLLGDYAEQFF | 24 |
| 1311 | CASSLLGNTLYF | 24 |
| 1312 | CASSLLGSQNTLYF | 24 |
| 1313 | CASSLNNQDTQYF | 24 |
| 1314 | CASSLNSGNTLYF | 24 |
| 1315 | CASSLQANTGQLYF | 24 |
| 1316 | CASSLQGDSDYTF | 24 |
| 1317 | CASSLQGDTEVFF | 24 |
| 1318 | CASSLQGETLYF | 24 |
| 1319 | CASSLQGGAEQFF | 24 |
| 1320 | CASSLQGSNERLFF | 24 |
| 1321 | CASSLQGTGQLYF | 24 |
| 1322 | CASSLQGYNSPLYF | 24 |
| 1323 | CASSLQNYAEQFF | 24 |
| 1324 | CASSLRGAETLYF | 24 |
| 1325 | CASSLRGDTEVFF | 24 |
| 1326 | CASSLRGNQDTQYF | 24 |
| 1327 | CASSLRGNTGQLYF | 24 |
| 1328 | CASSLRGNTLYF | 24 |
| 1329 | CASSLRGNYAEQFF | 24 |
| 1330 | CASSLRGSAETLYF | 24 |
| 1331 | CASSLSETLYF | 24 |
| 1332 | CASSLSGDQDTQYF | 24 |
| 1333 | CASSLSGNQDTQYF | 24 |
| 1334 | CASSLTGNSDYTF | 24 |
| 1335 | CASSLTISNERLFF | 24 |
| 1336 | CASSLTNSDYTF | 24 |
| 1337 | CASSLTSSQNTLYF | 24 |
| 1338 | CASSLTVSNERLFF | 24 |
| 1339 | CASSLVAETLYF | 24 |
| 1340 | CASSLVGDQDTQYF | 24 |
| 1341 | CASSINGGAETLYF | 24 |
| 1342 | CASSINGGQNTLYF | 24 |
| 1343 | CASSLWGSAETLYF | 24 |
| 1344 | CASSNSGNTLYF | 24 |
| 1345 | CASSNSQNTLYF | 24 |
| 1346 | CASSPDRGQNTLYF | 24 |
| 1347 | CASSPDSNQDTQYF | 24 |
| 1348 | CASSPDSYAEQFF | 24 |
| 1349 | CASSPDWGAETLYF | 24 |
| 1350 | CASSPDWGSAETLYF | 24 |
| 1351 | CASSPDWGYAEQFF | 24 |
| 1352 | CASSPDYAEQFF | 24 |
| 1353 | CASSPGANTEVFF | 24 |
| 1354 | CASSPGLGAETLYF | 24 |
| 1355 | CASSPGLGENTLYF | 24 |
| 1356 | CASSPGLGYAEQFF | 24 |
| 1357 | CASSPGLNTGQLYF | 24 |
| 1358 | CASSPGLNYAEQFF | 24 |
| 1359 | CASSPGLQDTQYF | 24 |
| 1360 | CASSPGNYAEQFF | 24 |
| 1361 | CASSPGQNERLFF | 24 |
| 1362 | CASSPGQSAETLYF | 24 |
| 1363 | CASSPGTENTLYF | 24 |
| 1364 | CASSPGTGAETLYF | 24 |
| 1365 | CASSPGTSQNTLYF | 24 |
| 1366 | CASSPLGNYAEQFF | 24 |
| 1367 | CASSPNTEVFF | 24 |
| 1368 | CASSPQGSGNTLYF | 24 |
| 1369 | CASSPQNTLYF | 24 |
| 1370 | CASSPRAETLYF | 24 |
| 1371 | CASSPRDNYAEQFF | 24 |
| 1372 | CASSPTASAETLYF | 24 |
| 1373 | CASSPTGDQDTQYF | 24 |
| 1374 | CASSPTSQNTLYF | 24 |
| 1375 | CASSQANSDYTF | 24 |
| 1376 | CASSQDNYAEQFF | 24 |
| 1377 | CASSQDSGNTLYF | 24 |
| 1378 | CASSQEGAETLYF | 24 |
| 1379 | CASSQRDWCYAEQFF | 24 |
| 1380 | CASSQSQNTLYF | 24 |
| 1381 | CASSRANTGQLYF | 24 |
| 1382 | CASSRDISAETLYF | 24 |
| 1383 | CASSRDNNNQAPLF | 24 |
| 1384 | CASSRDRAETLYF | 24 |
| 1385 | CASSRDRGAETLYF | 24 |
| 1386 | CASSRDRGNSDYTF | 24 |
| 1387 | CASSRDRGQNTLYF | 24 |
| 1388 | CASSRDRGYAEQFF | 24 |
| 1389 | CASSRDSGNTLYF | 24 |
| 1390 | CASSRDTYAEQFF | 24 |
| 1391 | CASSRDWGNQDTQYF | 24 |
| 1392 | CASSRGQNYAEQFF | 24 |
| 1393 | CASSRLGDQDTQYF | 24 |
| 1394 | CASSRLGDYAEQFF | 24 |
| 1395 | CASSRQDQDTQYF | 24 |
| 1396 | CASSRQGDTGQLYF | 24 |
| 1397 | CASSRQGSAETLYF | 24 |
| 1398 | CASSRQGSGNTLYF | 24 |
| 1399 | CASSRQGTEVFF | 24 |
| 1400 | CASSRQLSNERLFF | 24 |
| 1401 | CASSRQNTLYF | 24 |
| 1402 | CASSRQSNTEVFF | 24 |
| 1403 | CASSRQTNTEVFF | 24 |
| 1404 | CASSRSAETLYF | 24 |
| 1405 | CASSRTANTEVFF | 24 |
| 1406 | CASSRTENTLYF | 24 |
| 1407 | CASSRTGAETLYF | 24 |
| 1408 | CASSRTGGNYAEQFF | 24 |
| 1409 | CASSRTGGSQNTLYF | 24 |
| 1410 | CASSRTGNYAEQFF | 24 |
| 1411 | CASSRTSAETLYF | 24 |
| 1412 | CASSSDAEQFF | 24 |
| 1413 | CASSSGQNTGQLYF | 24 |
| 1414 | CASSSGTGNERLFF | 24 |
| 1415 | CASSSNSGNTLYF | 24 |
| 1416 | CASSSNTEVFF | 24 |
| 1417 | CASSSQGSGNTLYF | 24 |
| 1418 | CASSSQGSQNTLYF | 24 |
| 1419 | CASSSQGTEVFF | 24 |
| 1420 | CASSSSSAETLYF | 24 |
| 1421 | CASSSSSQNTLYF | 24 |
| 1422 | CASSSTANTGQLYF | 24 |
| 1423 | CASSSTGNTGQLYF | 24 |
| 1424 | CASSSTSQNTLYF | 24 |
| 1425 | CASSTGDQDTQYF | 24 |
| 1426 | CASSTGGAETLYF | 24 |
| 1427 | CASSTGGQNTLYF | 24 |
| 1428 | CASSTGGYAEQFF | 24 |
| 1429 | CASSTGNTEVFF | 24 |
| 1430 | CASSIGSQNTLYF | 24 |
| 1431 | CASSTNTGQLYF | 24 |
| 1432 | CASSWDRNTEVFF | 24 |
| 1433 | CGARDHTSNTEVFF | 24 |
| 1434 | CGARDWGNTGQLYF | 24 |
| 1435 | CGARDWGSQNTLYF | 24 |
| 1436 | CSADSQNTLYF | 24 |
| 1437 | CASGDAGANTEVFF | 23 |
| 1438 | CASGDAGGNTEVFF | 23 |
| 1439 | CASGDAGGNTGQLYF | 23 |
| 1440 | CASGDAGGSQNTLYF | 23 |
| 1441 | CASGDAGSGNTLYF | 23 |
| 1442 | CASGDAGTANTEVFF | 23 |
| 1443 | CASGDAGVQDTQYF | 23 |
| 1444 | CASGDAQDTQYF | 23 |
| 1445 | CASGDAQSQNTLYF | 23 |
| 1446 | CASGDASSGNTLYF | 23 |
| 1447 | CASGDATTSAETLYF | 23 |
| 1448 | CASGDAYNSPLYF | 23 |
| 1449 | CASGDGGNQDTQYF | 23 |
| 1450 | CASGDGNTEVFF | 23 |
| 1451 | CASGDGTTNTEVFF | 23 |
| 1452 | CASGDNQAPLF | 23 |
| 1453 | CASGDNQDTQYF | 23 |
| 1454 | CASGDNSAETLYF | 23 |
| 1455 | CASGDQNTLYF | 23 |
| 1456 | CASGDRANSDYTF | 23 |
| 1457 | CASGDRDTEVFF | 23 |
| 1458 | CASGDRGNYAEQFF | 23 |
| 1459 | CASGDRNSDYTF | 23 |
| 1460 | CASGDSYNSPLYF | 23 |
| 1461 | CASGDVGSQNTLYF | 23 |
| 1462 | CASGDWGAETLYF | 23 |
| 1463 | CASGDWGGYAEQFF | 23 |
| 1464 | CASGDWGQDTQYF | 23 |
| 1465 | CASGEQDTQYF | 23 |
| 1466 | CASGESQNTLYF | 23 |
| 1467 | CASGETANTEVFF | 23 |
| 1468 | CASGNQDTQYF | 23 |
| 1469 | CASGTGNYAEQFF | 23 |
| 1470 | CASRDSGNTLYF | 23 |
| 1471 | CASRDSSAETLYF | 23 |
| 1472 | CASRSAETLYF | 23 |
| 1473 | CASSAGSQNTLYF | 23 |
| 1474 | CASSANTGQLYF | 23 |
| 1475 | CASSDAETLYF | 23 |
| 1476 | CASSDAGAETLYF | 23 |
| 1477 | CASSDAGNTEVFF | 23 |
| 1478 | CASSDASSGNTLYF | 23 |
| 1479 | CASSDGSAETLYF | 23 |
| 1480 | CASSDNERLFF | 23 |
| 1481 | CASSDNSDYTF | 23 |
| 1482 | CASSDNTGQLYF | 23 |
| 1483 | CASSDRANTEVFF | 23 |
| 1484 | CASSDRDSGNTLYF | 23 |
| 1485 | CASSDRNERLFF | 23 |
| 1486 | CASSDRSQNTLYF | 23 |
| 1487 | CASSDSSYNSPLYF | 23 |
| 1488 | CASSDTGQLYF | 23 |
| 1489 | CASSDWGQDTQYF | 23 |
| 1490 | CASSDWGSQNTLYF | 23 |
| 1491 | CASSEQGNTEVFF | 23 |
| 1492 | CASSESAETLYF | 23 |
| 1493 | CASSFDTGQLYF | 23 |
| 1494 | CASSFGQNTEVFF | 23 |
| 1495 | CASSFNSAETLYF | 23 |
| 1496 | CASSFQNTEVFF | 23 |
| 1497 | CASSFSNERLFF | 23 |
| 1498 | CASSFTGGQNTLYF | 23 |
| 1499 | CASSFWGNYAEQFF | 23 |
| 1500 | CASSGDWGNYAEQFF | 23 |
| 1501 | CASSGQNTEVFF | 23 |
| 1502 | CASSGTGGQDTQYF | 23 |
| 1503 | CASSGTNTEVFF | 23 |
| 1504 | CASSHNQDTQYF | 23 |
| 1505 | CASSINSAETLYF | 23 |
| 1506 | CASSIRGNTEVFF | 23 |
| 1507 | CASSLAGGQNTLYF | 23 |
| 1508 | CASSLAGNSDYTF | 23 |
| 1509 | CASSLDGNQDTQYF | 23 |
| 1510 | CASSLDGNYAEQFF | 23 |
| 1511 | CASSLDNQAPLF | 23 |
| 1512 | CASSLDSANTEVFF | 23 |
| 1513 | CASSLDSTEVFF | 23 |
| 1514 | CASSLDWGDAEQFF | 23 |
| 1515 | CASSLEAETLYF | 23 |
| 1516 | CASSLEGGNYAEQFF | 23 |
| 1517 | CASSLEQNTEVFF | 23 |
| 1518 | CASSLGAQNTLYF | 23 |
| 1519 | CASSLGASAETLYF | 23 |
| 1520 | CASSLGDNQDTQYF | 23 |
| 1521 | CASSLGDTLYF | 23 |
| 1522 | CASSLGGAGNTLYF | 23 |
| 1523 | CASSLGGGAETLYF | 23 |
| 1524 | CASSLGHYAEQFF | 23 |
| 1525 | CASSLGLGENTLYF | 23 |
| 1526 | CASSLGLSAETLYF | 23 |
| 1527 | CASSLGNERLFF | 23 |
| 1528 | CASSLGQANTGQLYF | 23 |
| 1529 | CASSLGQGAGNTLYF | 23 |
| 1530 | CASSLGQGNTGQLYF | 23 |
| 1531 | CASSLGQGNYAEQFF | 23 |
| 1532 | CASSLGQGQNTLYF | 23 |
| 1533 | CASSLGQNQAPLF | 23 |
| 1534 | CASSLGQYNSPLYF | 23 |
| 1535 | CASSLGRNTEVFF | 23 |
| 1536 | CASSLGSSGNTLYF | 23 |
| 1537 | CASSLGTGGAETLYF | 23 |
| 1538 | CASSLGTGNTEVFF | 23 |
| 1539 | CASSLGTTNERLFF | 23 |
| 1540 | CASSLLGNQDTQYF | 23 |
| 1541 | CASSLLGNTGQLYF | 23 |
| 1542 | CASSLLGSAETLYF | 23 |
| 1543 | CASSLLGTSAETLYF | 23 |
| 1544 | CASSLNNYAEQFF | 23 |
| 1545 | CASSLNYAEQFF | 23 |
| 1546 | CASSLQENTLYF | 23 |
| 1547 | CASSLQGANERLFF | 23 |
| 1548 | CASSLQGDTGQLYF | 23 |
| 1549 | CASSLQGGAETLYF | 23 |
| 1550 | CASSLQGGQNTLYF | 23 |
| 1551 | CASSLQGGTEVFF | 23 |
| 1552 | CASSLQGGYAEQFF | 23 |
| 1553 | CASSLQGNQAPLF | 23 |
| 1554 | CASSLQGTNERLFF | 23 |
| 1555 | CASSLRANTEVFF | 23 |
| 1556 | CASSLRDNYAEQFF | 23 |
| 1557 | CASSLRDSGNTLYF | 23 |
| 1558 | CASSLRGGQNTLYF | 23 |
| 1559 | CASSLRGNSDYTF | 23 |
| 1560 | CASSLSANSDYTF | 23 |
| 1561 | CASSLSANTEVFF | 23 |
| 1562 | CASSLSGTEVFF | 23 |
| 1563 | CASSLSNSGNTLYF | 23 |
| 1564 | CASSLTGANSDYTF | 23 |
| 1565 | CASSLTGDSDYTF | 23 |
| 1566 | CASSLTGDTEVFF | 23 |
| 1567 | CASSLTGDTGQLYF | 23 |
| 1568 | CASSLTGGNYAEQFF | 23 |
| 1569 | CASSLTGNQDTQYF | 23 |
| 1570 | CASSLTGYAEQFF | 23 |
| 1571 | CASSLVGNTGQLYF | 23 |
| 1572 | CASSLVGQNTLYF | 23 |
| 1573 | CASSLVTGQLYF | 23 |
| 1574 | CASSPDTEVFF | 23 |
| 1575 | CASSPGANTGQLYF | 23 |
| 1576 | CASSPGGSAETLYF | 23 |
| 1577 | CASSPGGYAEQFF | 23 |
| 1578 | CASSPGLGQNTLYF | 23 |
| 1579 | CASSPGLGSQNTLYF | 23 |
| 1580 | CASSPGNQDTQYF | 23 |
| 1581 | CASSPGNTGQLYF | 23 |
| 1582 | CASSPGQGNERLFF | 23 |
| 1583 | CASSPGQKNTLYF | 23 |
| 1584 | CASSPGQNQAPLF | 23 |
| 1585 | CASSPGTANERLFF | 23 |
| 1586 | CASSPGTDTEVFF | 23 |
| 1587 | CASSPNSAETLYF | 23 |
| 1588 | CASSPNSQNTLYF | 23 |
| 1589 | CASSPQGANTEVFF | 23 |
| 1590 | CASSPQGNSDYTF | 23 |
| 1591 | CASSPRGAETLYF | 23 |
| 1592 | CASSPSSQNTLYF | 23 |
| 1593 | CASSPTGGAETLYF | 23 |
| 1594 | CASSPTGSAETLYF | 23 |
| 1595 | CASSPTINQDTQYF | 23 |
| 1596 | CASSPTTNTEVFF | 23 |
| 1597 | CASSPTVNQDTQYF | 23 |
| 1598 | CASSQANTEVFF | 23 |
| 1599 | CASSQDGSQNTLYF | 23 |
| 1600 | CASSQDNSGNTLYF | 23 |
| 1601 | CASSQDRGSAETLYF | 23 |
| 1602 | CASSQDSYNSPLYF | 23 |
| 1603 | CASSQDWGQNTLYF | 23 |
| 1604 | CASSQGANSDYTF | 23 |
| 1605 | CASSQGANTEVFF | 23 |
| 1606 | CASSQGNTEVFF | 23 |
| 1607 | CASSQGNTLYF | 23 |
| 1608 | CASSQGTANSDYTF | 23 |
| 1609 | CASSQQGSAETLYF | 23 |
| 1610 | CASSQQGTEVFF | 23 |
| 1611 | CASSQTANSDYTF | 23 |
| 1612 | CASSQTGGQNTLYF | 23 |
| 1613 | CASSRDKNTGQLYF | 23 |
| 1614 | CASSRDNNQAPLF | 23 |
| 1615 | CASSRDNNQDTQYF | 23 |
| 1616 | CASSRDRYAEQFF | 23 |
| 1617 | CASSRDSSNERLFF | 23 |
| 1618 | CASSRDTNTEVFF | 23 |
| 1619 | CASSRDTQDTQYF | 23 |
| 1620 | CASSRDWGDTQYF | 23 |
| 1621 | CASSRDWGSYEQYF | 23 |
| 1622 | CASSRDWNYAEQFF | 23 |
| 1623 | CASSRDWSAETLYF | 23 |
| 1624 | CASSRDYAEQFF | 23 |
| 1625 | CASSRGNTEVFF | 23 |
| 1626 | CASSRGQNTEVFF | 23 |
| 1627 | CASSRLGANTGQLYF | 23 |
| 1628 | CASSRLGENTLYF | 23 |
| 1629 | CASSRLGSSAETLYF | 23 |
| 1630 | CASSRQANTGQLYF | 23 |
| 1631 | CASSRQGETLYF | 23 |
| 1632 | CASSRQGEVFF | 23 |
| 1633 | CASSRQGYAEQFF | 23 |
| 1634 | CASSRQQNTLYF | 23 |
| 1635 | CASSRQSAETLYF | 23 |
| 1636 | CASSRQYAEQFF | 23 |
| 1637 | CASSRTASQNTLYF | 23 |
| 1638 | CASSRTGGNTLYF | 23 |
| 1639 | CASSRTGNTEVFF | 23 |
| 1640 | CASSRTGNTLYF | 23 |
| 1641 | CASSRTISNERLFF | 23 |
| 1642 | CASSRTTSAETLYF | 23 |
| 1643 | CASSSANTEVFF | 23 |
| 1644 | CASSSDSSQNTLYF | 23 |
| 1645 | CASSSDWGQNTLYF | 23 |
| 1646 | CASSSGANTEVFF | 23 |
| 1647 | CASSSGGTEVFF | 23 |
| 1648 | CASSSGQNYAEQFF | 23 |
| 1649 | CASSSGTANSDYTF | 23 |
| 1650 | CASSSGTTNSDYTF | 23 |
| 1651 | CASSSQGAGNTLYF | 23 |
| 1652 | CASSSQGYAEQFF | 23 |
| 1653 | CASSSTGDTGQLYF | 23 |
| 1654 | CASSSTGNSDYTF | 23 |
| 1655 | CASSTGGNYAEQFF | 23 |
| 1656 | CASSTGTANTEVFF | 23 |
| 1657 | CASSVNQDTQYF | 23 |
| 1658 | CASSVSAETLYF | 23 |
| 1659 | CASSWGDQDTQYF | 23 |
| 1660 | CASSWTDSGNTLYF | 23 |
| 1661 | CAWSLGDQDTQYF | 23 |
| 1662 | CAWSLGGNYAEQFF | 23 |
| 1663 | CAWSLGGQDTQYF | 23 |
| 1664 | CAWSLGSAETLYF | 23 |
| 1665 | CAWSLSAETLYF | 23 |
| 1666 | CGARDNYAEQFF | 23 |
| 1667 | CGARDRNTGQLYF | 23 |
| 1668 | CGARDSQNTLYF | 23 |
| 1669 | CGARDWGSAETLYF | 23 |
| 1670 | CSADTEVFF | 23 |
| 1671 | CASGDAANSDYTF | 22 |
| 1672 | CASGDAGEDTQYF | 22 |
| 1673 | CASGDAGGEQYF | 22 |
| 1674 | CASGDAGGNQDTQYF | 22 |
| 1675 | CASGDAGISNERLFF | 22 |
| 1676 | CASGDAGQNSDYTF | 22 |
| 1677 | CASGDAGVSYEQYF | 22 |
| 1678 | CASGDARYEQYF | 22 |
| 1679 | CASGDAWGQDIQYF | 22 |
| 1680 | CASGDDRGQNTLYF | 22 |
| 1681 | CASGDGGNTEVFF | 22 |
| 1682 | CASGDGGSQNTLYF | 22 |
| 1683 | CASGDGSAETLYF | 22 |
| 1684 | CASGDGTANSDYTF | 22 |
| 1685 | CASGDLGNYAEQFF | 22 |
| 1686 | CASGDNNERLFF | 22 |
| 1687 | CASGDNSDYTF | 22 |
| 1688 | CASGDPANSDYTF | 22 |
| 1689 | CASGDRDNYAEQFF | 22 |
| 1690 | CASGDRGNIGQLYF | 22 |
| 1691 | CASGDRGQDTQYF | 22 |
| 1692 | CASGDSANTEVFF | 22 |
| 1693 | CASGDTSAETLYF | 22 |
| 1694 | CASGEEDTQYF | 22 |
| 1695 | CASGETGNTEVFF | 22 |
| 1696 | CASGETGNYAEQFF | 22 |
| 1697 | CASGETLYF | 22 |
| 1698 | CASGPGQGYAEQFF | 22 |
| 1699 | CASGSQNTLYF | 22 |
| 1700 | CASGTANTEVFF | 22 |
| 1701 | CASGTGNSDYTF | 22 |
| 1702 | CASRDNANTEVFF | 22 |
| 1703 | CASRDNQDTQYF | 22 |
| 1704 | CASRGQGNTEVFF | 22 |
| 1705 | CASRQGANTEVFF | 22 |
| 1706 | CASRQNTEVFF | 22 |
| 1707 | CASRSQNTLYF | 22 |
| 1708 | CASSADSAETLYF | 22 |
| 1709 | CASSAGTANTEVFF | 22 |
| 1710 | CASSDAGSAETLYF | 22 |
| 1711 | CASSDGGNIEVFF | 22 |
| 1712 | CASSDGTANSDYTF | 22 |
| 1713 | CASSDNSAETLYF | 22 |
| 1714 | CASSDPGQDTQYF | 22 |
| 1715 | CASSDRDQDTQYF | 22 |
| 1716 | CASSDRETLYF | 22 |
| 1717 | CASSDRGQDTQYF | 22 |
| 1718 | CASSDRGSAETLYF | 22 |
| 1719 | CASSDTANTEVFF | 22 |
| 1720 | CASSEDTEVFF | 22 |
| 1721 | CASSEGAGNTLYF | 22 |
| 1722 | CASSFDNSGNTLYF | 22 |
| 1723 | CASSFDRNTEVFF | 22 |
| 1724 | CASSFGERLFF | 22 |
| 1725 | CASSFLGNYAEQFF | 22 |
| 1726 | CASSFNERLFF | 22 |
| 1727 | CASSFQANSDYTF | 22 |
| 1728 | CASSFQNYAEQFF | 22 |
| 1729 | CASSFRIENTEVFF | 22 |
| 1730 | CASSFSSGNTLYF | 22 |
| 1731 | CASSFTANTEVFF | 22 |
| 1732 | CASSGGAETLYF | 22 |
| 1733 | CASSGNTLYF | 22 |
| 1734 | CASSGQNTGQLYF | 22 |
| 1735 | CASSGTDYAEQFF | 22 |
| 1736 | CASSGTGGNTLYF | 22 |
| 1737 | CASSGTGNTLYF | 22 |
| 1738 | CASSGTSAETLYF | 22 |
| 1739 | CASSGTSSAETLYF | 22 |
| 1740 | CASSGTTSAETLYF | 22 |
| 1741 | CASSHNYAEQFF | 22 |
| 1742 | CASSIGAETLYF | 22 |
| 1743 | CASSIGGNYAEQFF | 22 |
| 1744 | CASSLAGGAETLYF | 22 |
| 1745 | CASSLAQNTEVFF | 22 |
| 1746 | CASSLAQNTLYF | 22 |
| 1747 | CASSLASSGNTLYF | 22 |
| 1748 | CASSLDGGSAETLYF | 22 |
| 1749 | CASSLDGNSDYTF | 22 |
| 1750 | CASSLDGNTGQLYF | 22 |
| 1751 | CASSLDGSAETLYF | 22 |
| 1752 | CASSLDKEVFF | 22 |
| 1753 | CASSLDNNERLFF | 22 |
| 1754 | CASSLDNNQAPLF | 22 |
| 1755 | CASSLDRANTEVFF | 22 |
| 1756 | CASSLDRGQNTLYF | 22 |
| 1757 | CASSLDRGYAEQFF | 22 |
| 1758 | CASSLDSNTGQLYF | 22 |
| 1759 | CASSLDSSNERLFF | 22 |
| 1760 | CASSLDWGAETLYF | 22 |
| 1761 | CASSLEETLYF | 22 |
| 1762 | CASSLEGAQDTQYF | 22 |
| 1763 | CASSLEGASQNTLYF | 22 |
| 1764 | CASSLEGGAETLYF | 22 |
| 1765 | CASSLEGGNTLYF | 22 |
| 1766 | CASSLEGNSGNTLYF | 22 |
| 1767 | CASSLEGRQNTLYF | 22 |
| 1768 | CASSLEGTGNTLYF | 22 |
| 1769 | CASSLEQGAETLYF | 22 |
| 1770 | CASSLEQNTGQLYF | 22 |
| 1771 | CASSLEQYAEQFF | 22 |
| 1772 | CASSLGGENTLYF | 22 |
| 1773 | CASSLGGETLYF | 22 |
| 1774 | CASSLGGRAETLYF | 22 |
| 1775 | CASSLGHTEVFF | 22 |
| 1776 | CASSLGLGNYAEQFF | 22 |
| 1777 | CASSLGLGQNTLYF | 22 |
| 1778 | CASSLGLNQDTQYF | 22 |
| 1779 | CASSLGLNYAEQFF | 22 |
| 1780 | CASSLGLSQNTLYF | 22 |
| 1781 | CASSLGPNTEVFF | 22 |
| 1782 | CASSLGQGNERLFF | 22 |
| 1783 | CASSLGQNERLFF | 22 |
| 1784 | CASSLGSQDTQYF | 22 |
| 1785 | CASSLGTAETLYF | 22 |
| 1786 | CASSLGTDYAEQFF | 22 |
| 1787 | CASSLGTGYAEQFF | 22 |
| 1788 | CASSLGTNQDTQYF | 22 |
| 1789 | CASSLGTTSAETLYF | 22 |
| 1790 | CASSLGTYAEQFF | 22 |
| 1791 | CASSLLGAETLYF | 22 |
| 1792 | CASSLEGENTLYF | 22 |
| 1793 | CASSLLGGQNTLYF | 22 |
| 1794 | CASSLNAETLYF | 22 |
| 1795 | CASSLNNERLFF | 22 |
| 1796 | CASSLNTLYF | 22 |
| 1797 | CASSLPGSQNTLYF | 22 |
| 1798 | CASSLQGERLFF | 22 |
| 1799 | CASSLQNSGNTLYF | 22 |
| 1800 | CASSLQYAEQFF | 22 |
| 1801 | CASSLRANTGQLYF | 22 |
| 1802 | CASSERDTLYF | 22 |
| 1803 | CASSLRGYAEQFF | 22 |
| 1804 | CASSLRNSGNTLYF | 22 |
| 1805 | CASSLRNTEVFF | 22 |
| 1806 | CASSLRQNTLYF | 22 |
| 1807 | CASSLSGNSDYTF | 22 |
| 1808 | CASSLSNSDYTF | 22 |
| 1809 | CASSLSTNSDYTF | 22 |
| 1810 | CASSLSVNQDTQYF | 22 |
| 1811 | CASSLTGSGNTLYF | 22 |
| 1812 | CASSLTGTEVFF | 22 |
| 1813 | CASSLTPNTEVFF | 22 |
| 1814 | CASSLTTNTGQLYF | 22 |
| 1815 | CASSLVANSDYTF | 22 |
| 1816 | CASSLVGSAETLYF | 22 |
| 1817 | CASSLVNSGNTLYF | 22 |
| 1818 | CASSLVQNTLYF | 22 |
| 1819 | CASSPANTGQLYF | 22 |
| 1820 | CASSPDNSQNTLYF | 22 |
| 1821 | CASSPDTSQNTLYF | 22 |
| 1822 | CASSPGDTEVFF | 22 |
| 1823 | CASSPGHQDTQYF | 22 |
| 1824 | CASSPGLGSAETLYF | 22 |
| 1825 | CASSPGLSQNTLYF | 22 |
| 1826 | CASSPGQGNTLYF | 22 |
| 1827 | CASSPGQISNERLFF | 22 |
| 1828 | CASSPGQNNQAPLF | 22 |
| 1829 | CASSPGTGNSDYTF | 22 |
| 1830 | CASSPGTGNTEVFF | 22 |
| 1831 | CASSPQGDTGQLYF | 22 |
| 1832 | CASSPQGNYAEQFF | 22 |
| 1833 | CASSPQNTEVFF | 22 |
| 1834 | CASSPRDSAETLYF | 22 |
| 1835 | CASSPRLGQDTQYF | 22 |
| 1836 | CASSPSGNTLYF | 22 |
| 1837 | CASSPTGNTEVFF | 22 |
| 1838 | CASSPTGYAEQFF | 22 |
| 1839 | CASSPTISNERLFF | 22 |
| 1840 | CASSPTNSGNTLYF | 22 |
| 1841 | CASSPTNTEVFF | 22 |
| 1842 | CASSPWGNYAEQFF | 22 |
| 1843 | CASSQDRANTEVFF | 22 |
| 1844 | CASSQDSNQDTQYF | 22 |
| 1845 | CASSQDWDQDTQYF | 22 |
| 1846 | CASSQDWGSAETLYF | 22 |
| 1847 | CASSQEGNQDTQYF | 22 |
| 1848 | CASSQEGNTEVFF | 22 |
| 1849 | CASSQEGQQDTQYF | 22 |
| 1850 | CASSQGNSGNTLYF | 22 |
| 1851 | CASSQGQQNTLYF | 22 |
| 1852 | CASSQGTASAETLYF | 22 |
| 1853 | CASSQGTISNERLFF | 22 |
| 1854 | CASSQNYAEQFF | 22 |
| 1855 | CASSQQGAGNTLYF | 22 |
| 1856 | CASSQTGNTGQLYF | 22 |
| 1857 | CASSRDINQDTQYF | 22 |
| 1858 | CASSRDISGNTLYF | 22 |
| 1859 | CASSRDKNTEVFF | 22 |
| 1860 | CASSRDNANSDYTF | 22 |
| 1861 | CASSRDRGNTEVFF | 22 |
| 1862 | CASSRDRGTEVFF | 22 |
| 1863 | CASSRDRSQNTLYF | 22 |
| 1864 | CASSRDSNTEVFF | 22 |
| 1865 | CASSRDTNYAEQFF | 22 |
| 1866 | CASSRDWEDTQYF | 22 |
| 1867 | CASSRDWGAETLYT | 22 |
| 1868 | CASSRDWGQNTLYF | 22 |
| 1869 | CASSRGDQDTQYF | 22 |
| 1870 | CASSRGGQNTLYF | 22 |
| 1871 | CASSRGQGYAEQFF | 22 |
| 1872 | CASSRNYAEQFF | 22 |
| 1873 | CASSRQGDSDYTF | 22 |
| 1874 | CASSRQNQAPLF | 22 |
| 1875 | CASSRTAETLYF | 22 |
| 1876 | CASSRTGGNTEVFF | 22 |
| 1877 | CASSSDSSGNTLYF | 22 |
| 1878 | CASSSGENTLYF | 22 |
| 1879 | CASSSGGNYAEQFF | 22 |
| 1880 | CASSSGGSAETLYF | 22 |
| 1881 | CASSSGGSQNTLYF | 22 |
| 1882 | CASSSGLGQDTQYF | 22 |
| 1883 | CASSSGNTGQLYF | 22 |
| 1884 | CASSSGQKNTLYF | 22 |
| 1885 | CASSSGQSQNTLYF | 22 |
| 1886 | CASSSGQYAEQFF | 22 |
| 1887 | CASSSGSSAETLYF | 22 |
| 1888 | CASSSGTGAETLYF | 22 |
| 1889 | CASSSLGDQDTQYF | 22 |
| 1890 | CASSSNSAETLYF | 22 |
| 1891 | CASSSNSDYTF | 22 |
| 1892 | CASSSNSQNTLYF | 22 |
| 1893 | CASSSQGNQDTQYF | 22 |
| 1894 | CASSSQNTEVFF | 22 |
| 1895 | CASSSQNYAEQFF | 22 |
| 1896 | CASSSTGSQNTLYF | 22 |
| 1897 | CASSSTNSDYTF | 22 |
| 1898 | CASSSWGSQNTLYF | 22 |
| 1899 | CASSTGNYAEQFF | 22 |
| 1900 | CASSTGSAETLYF | 22 |
| 1901 | CASSWGGQNTLYF | 22 |
| 1902 | CASSWTANTGQLYF | 22 |
| 1903 | CASSYGAETLYF | 22 |
| 1904 | CASSYGGQNTLYF | 22 |
| 1905 | CASSYNNQAPLF | 22 |
| 1906 | CASSYQANTEVFF | 22 |
| 1907 | CASSYRNTEVFF | 22 |
| 1908 | CASSYWGNYAEQFF | 22 |
| 1909 | CAWSLGGQNTLYF | 22 |
| 1910 | CAWSLGNYAEQFF | 22 |
| 1911 | CAWSLGSSAETLYF | 22 |
| 1912 | CAWSLGYEQYF | 22 |
| 1913 | CGARDNSGNTLYF | 22 |
| 1914 | CGARDSSQNTLYF | 22 |
| 1915 | CGARDSSYNSPLYF | 22 |
| 1916 | CGARDWGSSYEQYF | 22 |
| 1917 | CGARQGNTEVFF | 22 |
| 1918 | CGARTVSNERLFF | 22 |
| 1919 | CSADAEQFF | 22 |
| 1920 | CSAGNSDYTF | 22 |
| 1921 | CSAGQNTEVFF | 22 |
| 1922 | CSAGSAETLYF | 22 |

Each possibility represents a separate embodiment of the present invention.

According to some embodiments of the present invention, the CDR3 sequence is selected from the group consisting of the sequences in Table 7.

According to some embodiments of the present invention, the peptide amino acid sequence is selected from the group consisting of SEQ ID NOs of Table 7.

TABLE 7

| SEQ | | sharing | SEQ | | sharing | SEQ | | sharing |
|---|---|---|---|---|---|---|---|---|
| ID | | level | ID | | level | ID | | level |
| NO: | sequence | in mice | NO: | sequence | in mice | NO: | sequence | in mice |
| 35 | CASSLEGEDTQYF | 28 | 665 | CASGDWGNYAEQFF | 27 | 1295 | CASSLENYAEQFF | 24 |
| 36 | CASSPGQQDTQYF | 28 | 666 | CASGENTLYF | 27 | 1296 | CASSLEQTEVFF | 24 |
| 37 | CASSFQDTQYF | 28 | 667 | CASGGQNYAEQFF | 27 | 1297 | CASSLGDYAEQFF | 24 |
| 38 | CASSRQQDTQYF | 28 | 668 | CASGGSQNTLYF | 27 | 1298 | CASSLGENTEVFF | 24 |
| 39 | CASSRDSQDTQYF | 28 | 669 | CASGQNTLYF | 27 | 1299 | CASSLGEVFF | 24 |
| 40 | CASSLQGYEQYF | 28 | 670 | CASKTANQDTQYF | 27 | 1300 | CASSLGGANTEVFF | 24 |
| 41 | CASSDSSYEQYF | 28 | 671 | CASRDNSGNTLYF | 27 | 1301 | CASSLGGTGQLYF | 24 |
| 42 | CASSLGSSYEQYF | 28 | 672 | CASRDSSQNTLYF | 27 | 1302 | CASSLGLYAEQFF | 24 |
| 43 | CASGDGDTQYF | 28 | 673 | CASRRDSAETLYF | 27 | 1303 | CASSLGNQAPLF | 24 |
| 44 | CASGDYEQYF | 28 | 674 | CASRTGGYAEQFF | 27 | 1304 | CASSLGQGNSDYTF | 24 |
| 45 | CASSLEDTQYF | 28 | 675 | CASRTSAETLYF | 27 | 1305 | CASSLGTENTLYF | 24 |
| 46 | CASSLEGDEQYF | 28 | 676 | CASSASAETLYF | 27 | 1306 | CASSLGTGAETLYF | 24 |
| 47 | CASSLGYEQYF | 28 | 677 | CASSDAGQNTLYF | 27 | 1307 | CASSLGTGNTGQLYF | 24 |
| 48 | CASSLGQYEQYF | 28 | 678 | CASSDANSDYTF | 27 | 1308 | CASSLGTNSDYTF | 24 |
| 49 | CASSVDGSYEQYF | 28 | 679 | CASSDGSQNTLYF | 27 | 1309 | CASSLGTSQNTLYF | 24 |
| 50 | CASSPQDTQYF | 28 | 680 | CASSDNQDTQYF | 27 | 1310 | CASSLLGDYAEQFF | 24 |
| 51 | CASSLDNYEQYF | 28 | 681 | CASSDNYAEQFF | 27 | 1311 | CASSLLGNTLYF | 24 |
| 52 | CASSLEGYEQYF | 28 | 682 | CASSDSSGNTLYF | 27 | 1312 | CASSLLGSQNTLYF | 24 |
| 53 | CASSLDEQYF | 28 | 683 | CASSDWGSAETLYF | 27 | 1313 | CASSLNNQDTQYF | 24 |
| 54 | CASSLEGNQDTQYF | 28 | 684 | CASSESQNTLYF | 27 | 1314 | CASSLNSGNTLYF | 24 |
| 55 | CASSLEGDTQYF | 28 | 685 | CASSFDSAETLYF | 27 | 1315 | CASSLQANTGQLYF | 24 |
| 56 | CASSLDYEQYF | 28 | 686 | CASSFDTEVFF | 27 | 1316 | CASSLQGDSDYTF | 24 |
| 57 | CASSLGDTQYF | 28 | 687 | CASSFGGQNTLYF | 27 | 1317 | CASSLQGDTEVFF | 24 |
| 58 | CASSLGEQYF | 28 | 688 | CASSFQNTLYF | 27 | 1318 | CASSLQGETLYF | 24 |
| 59 | CASSQDTQYF | 28 | 689 | CASSFSSAETLYF | 27 | 1319 | CASSLQGGAEQFF | 24 |
| 60 | CASSLDRYEQYF | 28 | 690 | CASSGAETLYF | 27 | 1320 | CASSLQGSNERLFF | 24 |
| 61 | CASSGTGGYEQYF | 28 | 691 | CASSGQANTEVFF | 27 | 1321 | CASSLQGTGQLYF | 24 |
| 62 | CASSSYEQYF | 28 | 692 | CASSGQNYAEQFF | 27 | 1322 | CASSLQGYNSPLYF | 24 |
| 63 | CASSSGQYEQYF | 28 | 693 | CASSGTASAETLYF | 27 | 1323 | CASSLQNYAEQFF | 24 |
| 64 | CASSLLGGAEQFF | 28 | 694 | CASSGTGGYAEQFF | 27 | 1324 | CASSLRGAETLYF | 24 |
| 65 | CASSLDRDEQYF | 28 | 695 | CASSGTISNERLFF | 27 | 1325 | CASSLRGDTEVFF | 24 |
| 66 | CASSLDQDTQYF | 28 | 696 | CASSGTVSNERLFF | 27 | 1326 | CASSLRGNQDTQYF | 24 |
| 67 | CASSLEGSSYEQYF | 28 | 697 | CASSLAETLYF | 27 | 1327 | CASSLRGNTGQLYF | 24 |
| 68 | CASSLGGYEQYF | 28 | 698 | CASSLAGGYAEQFF | 27 | 1328 | CASSLRGNTLYF | 24 |
| 69 | CASSLGAEQFF | 28 | 699 | CASSLAGSGNTLYF | 27 | 1329 | CASSLRGNYAEQFF | 24 |
| 70 | CASSLSGYEQYF | 28 | 700 | CASSLANSGNTLYF | 27 | 1330 | CASSLRGSAETLYF | 24 |
| 71 | CASSSSYEQYF | 28 | 701 | CASSLASQNTLYF | 27 | 1331 | CASSLSETLYF | 24 |
| 72 | CASSLGQDTQYF | 28 | 702 | CASSLDAETLYF | 27 | 1332 | CASSLSGDQDTQYF | 24 |
| 73 | CASSLGGQDTQYF | 28 | 703 | CASSLDANSDYTF | 27 | 1333 | CASSLSGNQDTQYF | 24 |
| 74 | CASSFNQDTQYF | 28 | 704 | CASSLDNSDYTF | 27 | 1334 | CASSLTGNSDYTF | 24 |
| 75 | CASGQDTQYF | 28 | 705 | CASSLDNSQNTLYF | 27 | 1335 | CASSLTISNERLFF | 24 |
| 76 | CASSRDWGYEQYF | 28 | 706 | CASSLDNTEVFF | 27 | 1336 | CASSLTNSDYTF | 24 |
| 77 | CASSLDSYEQYF | 28 | 707 | CASSLDQNTLYF | 27 | 1337 | CASSLTSSQNTLYF | 24 |
| 78 | CASSRQYEQYF | 28 | 708 | CASSLDRDTEVFF | 27 | 1338 | CASSLTVSNERLFF | 24 |
| 79 | CASSLKDTQYF | 28 | 709 | CASSLDREVFF | 27 | 1339 | CASSLVAETLYF | 24 |
| 50 | CASSLGQNTEVFF | 28 | 710 | CASSLDRGAETLYF | 27 | 1340 | CASSLVGDQDTQYF | 24 |
| 81 | CASSFGTGDEQYF | 28 | 711 | CASSLDRNSDYTF | 27 | 1341 | CASSLVGGNETLYF | 24 |
| 82 | CASSLGEDTQYF | 28 | 712 | CASSLDRNTEVFF | 27 | 1342 | CASSLVGGQNTLYF | 24 |
| 83 | CASSRQNQDTQYF | 28 | 713 | CASSLDRNTGQLYF | 27 | 1343 | CASSLWGSAETLYF | 24 |
| 84 | CASSPSSYEQYF | 28 | 714 | CASSLDTNSDYTF | 27 | 1344 | CASSNSGNTLYF | 24 |
| 85 | CASSPDSYEQYF | 28 | 715 | CASSLDWGNTGQLYF | 27 | 1345 | CASSNSQNTLYF | 24 |
| 86 | CASSRDNYEQYF | 28 | 716 | CASSLDWGQNTLYF | 27 | 1346 | CASSPDRGQNTLYF | 24 |
| 87 | CASSRDSYEQYF | 28 | 717 | CASSLDWGSQNTLYF | 27 | 1347 | CASSPDSNQDTQYF | 24 |
| 88 | CASSLDRVEQYF | 28 | 718 | CASSLDWGYAEQFF | 27 | 1348 | CASSPDSYAEQFF | 24 |
| 89 | CASSLYAEQFF | 28 | 719 | CASSLDYAEQFF | 27 | 1349 | CASSPDWGAETLYF | 24 |
| 90 | CASSLDAEQFF | 28 | 720 | CASSLEANTEVFF | 27 | 1350 | CASSPDWGSAETLYF | 24 |
| 91 | CASSDSYEQYF | 28 | 721 | CASSLEGAGNTLYF | 27 | 1351 | CASSPDWGYAEQFF | 24 |
| 92 | CASSFGTEVFF | 28 | 722 | CASSLEGGQNTLYF | 27 | 1352 | CASSPDYAEQFF | 24 |
| 93 | CASSPDNYEQYF | 28 | 723 | CASSLEGSDYTF | 27 | 1353 | CASSPGANTEVFF | 24 |
| 94 | CASSPGQYEQYF | 28 | 724 | CASSLEGTGQLYF | 27 | 1354 | CASSPGLGAETLYF | 24 |
| 95 | CASSLQDTQYF | 28 | 725 | CASSLENTEVFF | 27 | 1355 | CASSPGLGENTLYF | 24 |
| 96 | CASSLRDTQYF | 28 | 726 | CASSLENTLYF | 27 | 1356 | CASSPGLGYAEQFF | 24 |
| 97 | CASSLGDEQYF | 28 | 727 | CASSLEQNSDYTF | 27 | 1357 | CASSPGLNTGQLYF | 24 |
| 98 | CASSLVAEQFF | 28 | 728 | CASSLETLYF | 27 | 1358 | CASSPGLNYAEQFF | 24 |
| 99 | CASSFSYEQYF | 28 | 729 | CASSLGANTEVFF | 27 | 1359 | CASSPGLQDTQYF | 24 |
| 100 | CASSLSYEQYF | 28 | 730 | CASSLGANTGQLYF | 27 | 1360 | CASSPGNYAEQFF | 24 |
| 101 | CASSDQDTQYF | 28 | 731 | CASSLGDTEVFF | 27 | 1361 | CASSPGQNERLFF | 24 |
| 102 | CASSDAGDTQYF | 28 | 732 | CASSLGGNTGQLYF | 27 | 1362 | CASSPGQSAETLYF | 24 |
| 103 | CASGDSYEQYF | 28 | 733 | CASSLGGNYAEQFF | 27 | 1363 | CASSPGTENTLYF | 24 |
| 104 | CASSLDTQYF | 28 | 734 | CASSLGGSGNTLYF | 27 | 1364 | CASSPGTGAETLYF | 24 |
| 105 | CASSFYAEQFF | 28 | 735 | CASSLGGTEVFF | 27 | 1365 | CASSPGTSQNTLYF | 24 |
| 106 | CASGDEQYF | 28 | 736 | CASSLGGYAEQFF | 27 | 1366 | CASSPLGNYAEQFF | 24 |
| 107 | CASGDAYEQYF | 28 | 737 | CASSLGNYAEQFF | 27 | 1367 | CASSPNTEVFF | 24 |
| 108 | CASSLYEQYF | 28 | 738 | CASSLGQANTEVFF | 27 | 1368 | CASSPQGSGNTLYF | 24 |
| 109 | CASSRDSSYEQYF | 28 | 739 | CASSLGQGAETLYF | 27 | 1369 | CASSPQNTLYF | 24 |
| 110 | CASSIRDTQYF | 28 | 740 | CASSLGQGNTEVFF | 27 | 1370 | CASSPRAETLYF | 24 |
| 111 | CASSRTGYEQYF | 27 | 741 | CASSLGQGYAEQFF | 27 | 1371 | CASSPRDNYAEQFF | 24 |
| 112 | CASSDAGYEQYF | 27 | 742 | CASSLGQYAEQFF | 27 | 1372 | CASSPTASAETLYF | 24 |
| 113 | CASSQQDTQYF | 27 | 743 | CASSLGTANTEVFF | 27 | 1373 | CASSPTGDQDTQYF | 24 |
| 114 | CASSGQQDTQYF | 27 | 744 | CASSLGTEVFF | 27 | 1374 | CASSPTSQNTLYF | 24 |
| 115 | CASGEDTQYF | 27 | 745 | CASSLGTNTEVFF | 27 | 1375 | CASSQANSDYTF | 24 |
| 116 | CASSRQDTQYF | 27 | 746 | CASSLGVNQDTQYF | 27 | 1376 | CASSQDNYAEQFF | 24 |
| 117 | CASSLGQQDTQYF | 27 | 747 | CASSLGVYAEQFF | 27 | 1377 | CASSQDSGNTLYF | 24 |
| 118 | CASSGGEQYF | 27 | 748 | CASSLLGGYAEQFF | 27 | 1378 | CASSQEGAETLYF | 24 |
| 119 | CASSYQDTQYF | 27 | 749 | CASSLNSDYTF | 27 | 1379 | CASSQRDWCYAEQFF | 24 |
| 120 | CASSLQYEQYF | 27 | 750 | CASSLNSQNTLYF | 27 | 1380 | CASSQSQNTLYF | 24 |
| 121 | CASSSQDTQYF | 27 | 751 | CASSLNTEVFF | 27 | 1381 | CASSRANTGQLYF | 24 |
| 122 | CASSQDRDTEVFF | 27 | 752 | CASSLQGANSDYTF | 27 | 1382 | CASSRDISAETLYF | 24 |
| 123 | CASSNQDTQYF | 27 | 753 | CASSLQGNTLYF | 27 | 1383 | CASSRDNNNQAPLF | 24 |
| 124 | CASSSSSYEQYF | 27 | 754 | CASSLQGNYAEQFF | 27 | 1384 | CASSRDRAETLYF | 24 |
| 125 | CASSLEGAEQFF | 27 | 755 | CASSLQGQNTLYF | 27 | 1385 | CASSRDRGAETLYF | 24 |
| 126 | CASSFGQYEQYF | 27 | 756 | CASSLQGYAEQFF | 27 | 1386 | CASSRDRGNSDYTF | 24 |
| 127 | CASSLTGDEQYF | 27 | 757 | CASSLQNSDYTF | 27 | 1387 | CASSRDRGQNTLYF | 24 |
| 128 | CASSRQGYEQYF | 27 | 758 | CASSLQNTEVFF | 27 | 1388 | CASSRDRGYAEQFF | 24 |
| 129 | CASSSTGYEQYF | 27 | 759 | CASSLRGQNTLYF | 27 | 1389 | CASSRDSGNTLYF | 24 |
| 130 | CASSLDGYEQYF | 27 | 760 | CASSLRSQNTLYF | 27 | 1390 | CASSRDTYAEQFF | 24 |
| 131 | CASSSNQDTQYF | 27 | 761 | CASSLSGGQNTLYF | 27 | 1391 | CASSRDWGNQDTQYF | 24 |
| 132 | CASSPTGDEQYF | 27 | 762 | CASSLSSAETLYF | 27 | 1392 | CASSRGQNYAEQFF | 24 |
| 133 | CASSLSAEQFF | 27 | 763 | CASSLTANTGQLYF | 27 | 1393 | CASSRLGDQDTQYF | 24 |
| 134 | CASSLDRGEQYF | 27 | 764 | CASSLTGAETLYF | 27 | 1394 | CASSRLGDYAEQFF | 24 |
| 135 | CASSLGYAEQFF | 27 | 765 | CASSLTGANTGQLYF | 27 | 1395 | CASSRQDQDTQYF | 24 |
| 136 | CASSSQGYEQYF | 27 | 766 | CASSLTGGQNTLYF | 27 | 1396 | CASSRQGDTGQLYF | 24 |
| 137 | CASSPSYEQYF | 27 | 767 | CASSLTTNTEVFF | 27 | 1397 | CASSRQGSAETLYF | 24 |
| 138 | CASSPDRYEQYF | 27 | 768 | CASSLTTSAETLYF | 27 | 1398 | CASSRQGSGNTLYF | 24 |
| 139 | CASSPNQDTQYF | 27 | 769 | CASSLVGNQDTQYF | 27 | 1399 | CASSRQGTEVFF | 24 |
| 140 | CASSLSSYEQYF | 27 | 770 | CASSLNGSQNTLYF | 27 | 1400 | CASSRQLSNERLFF | 24 |
| 141 | CASSLAGYEQYF | 27 | 771 | CASSNSAETLYF | 27 | 1401 | CASSRQNTLYF | 24 |
| 142 | CASSLTGGYEQYF | 27 | 772 | CASSPAETLYF | 27 | 1402 | CASSRQSNTEVFF | 24 |
| 143 | CASSLDIYEQYF | 27 | 773 | CASSPDSNERLFF | 27 | 1403 | CASSRQTNTEVFF | 24 |
| 144 | CASSLGGSSYEQYF | 27 | 774 | CASSPDWGSQNTLYF | 27 | 1404 | CASSRSAETLYF | 24 |
| 145 | CASGYEQYF | 27 | 775 | CASSPGAGSNERLFF | 27 | 1405 | CASSRTANTEVFF | 24 |
| 146 | CASGDADTQYF | 27 | 776 | CASSPGLGNYAEQFF | 27 | 1406 | CASSRTENTLYF | 24 |
| 147 | CASSLVGYEQYF | 27 | 777 | CASSPGLGQDTQYF | 27 | 1407 | CASSRTGAETLYF | 24 |
| 148 | CASGDEDTQYF | 27 | 778 | CASSPGNTEVFF | 27 | 1408 | CASSRTGGNYAEQFF | 24 |
| 149 | CASSQGQYEQYF | 27 | 779 | CASSPGQGAETLYF | 27 | 1409 | CASSRTGGSQNTLYF | 24 |
| 150 | CASSLDSSYEQYF | 27 | 780 | CASSPGQNTGQLYF | 27 | 1410 | CASSRTGNYAEQFF | 24 |
| 151 | CASSPTGYEQYF | 27 | 781 | CASSPGSAETLYF | 27 | 1411 | CASSRTSAETLYF | 24 |
| 152 | CASSQYEQYF | 27 | 782 | CASSPGTANTGQLYF | 27 | 1412 | CASSSDAEQFF | 24 |
| 153 | CASSRQGQDTQYF | 26 | 783 | CASSPGTTNSDYTF | 27 | 1413 | CASSSGQNTGQLYF | 24 |
| 154 | CASSLQGAEQFF | 26 | 784 | CASSPQGAETLYF | 27 | 1414 | CASSSGTGNERLFF | 24 |
| 155 | CASSDNYEQYF | 26 | 785 | CASSPQGSAETLYF | 27 | 1415 | CASSSNSGNTLYF | 24 |
| 156 | CASSDRGDTQYF | 26 | 786 | CASSPTANTEVFF | 27 | 1416 | CASSSNTEVFF | 24 |
| 157 | CASSDAEQFF | 26 | 787 | CASSPTGGQNTLYF | 27 | 1417 | CASSSQGSGNTLYF | 24 |
| 158 | CASSGQYEQYF | 26 | 788 | CASSQDSAETLYF | 27 | 1418 | CASSSQGSQNTLYF | 24 |
| 159 | CASSSGGQDTQYF | 26 | 789 | CASSQDSSGNTLYF | 27 | 1419 | CASSSQGTEVFF | 24 |
| 160 | CASSLGGAEQFF | 26 | 790 | CASSQDSSQNTLYF | 27 | 1420 | CASSSSSAETLYF | 24 |
| 161 | CASSPDAEQFF | 26 | 791 | CASSQDWGNYAEQFF | 27 | 1421 | CASSSSSQNTLYF | 24 |
| 162 | CASSLGGSYEQYF | 26 | 792 | CASSQGAGNTLYFF | 27 | 1422 | CASSSTANTGQLYF | 24 |
| 163 | CASSLAGDEQYF | 26 | 793 | CASSQNTLYF | 27 | 1423 | CASSSTGNTGQLYF | 24 |
| 164 | CASSLSGGYEQYF | 26 | 794 | CASSQQGNTEVFF | 27 | 1424 | CASSSTSQNTLYF | 24 |
| 165 | CASSYNQDTQYF | 26 | 795 | CASSQTSAETLYF | 27 | 1425 | CASSTGDQDTQYF | 24 |
| 166 | CASSLEGEQYF | 26 | 796 | CASSRAETLYF | 27 | 1426 | CASSTGGAETLYF | 24 |
| 167 | CASSRDRGYEQYF | 26 | 797 | CASSRDKNTLYF | 27 | 1427 | CASSTGGQNTLYF | 24 |
| 168 | CASSLGGEQYF | 26 | 798 | CASSRDNQNTLYF | 27 | 1428 | CASSTGGYAEQFF | 24 |
| 169 | CASSLEEQYF | 26 | 799 | CASSRDNSDYTF | 27 | 1429 | CASSTGNTEVFF | 24 |
| 170 | CASGDDEQYF | 26 | 800 | CASSRDNSQNTLYF | 27 | 1430 | CASSTGSQNTLYF | 24 |
| 171 | CASSRLPSYEQYF | 26 | 801 | CASSRDRDAEQFF | 27 | 1431 | CASSTNTGQLYF | 24 |
| 172 | CASSLAGGQDTQYF | 26 | 802 | CASSRDREVFF | 27 | 1432 | CASSWDRNTEVFF | 24 |
| 173 | CASSRTGGQDTQYF | 26 | 803 | CASSRDRGNTLYF | 27 | 1433 | CGARDHTSNTEVFF | 24 |
| 174 | CASSLEGQDTQYF | 26 | 804 | CASSRDRNTEVFF | 27 | 1434 | CGARDWGNTGQLYF | 24 |
| 175 | CASSLVGDEQYF | 26 | 805 | CASSRDSNERLFF | 27 | 1435 | CGARDWGSQNTLYF | 24 |
| 176 | CASSPQGYEQYF | 26 | 806 | CASSRDSNYAEQFF | 27 | 1436 | CSADSQNTLYF | 24 |
| 177 | CASSSDSYEQYF | 26 | 807 | CASSRDSSQNTLYF | 27 | 1437 | CASGDAGANTEVFF | 23 |
| 178 | CASSRTGEMQYF | 26 | 808 | CASSRDWGNTLYF | 27 | 1438 | CASGDAGGNTEVFF | 23 |
| 179 | CASSRTGGYEQYF | 26 | 809 | CASSRDWGSQNTLYF | 27 | 1439 | CASGDAGGNTGQLYF | 23 |
| 180 | CASSLQGSSYEQYF | 26 | 810 | CASSRDWGYAEQFF | 27 | 1440 | CASGDAGGSQNTLYF | 23 |
| 181 | CASSLTGNTEVFF | 26 | 811 | CASSRGSQNTLYF | 27 | 1441 | CASGDAGSGNTLYF | 23 |
| 182 | CASSLGGDTQYF | 26 | 812 | CASSRLGNYAEQFF | 27 | 1442 | CASGDAGTANTEVFF | 23 |
| 183 | CASSLDWGYEQYF | 26 | 813 | CASSRQGAETLYF | 27 | 1443 | CASGDAGVQDTQYF | 23 |
| 184 | CASSPGQSSYEQYF | 26 | 814 | CASSRQGNTLYF | 27 | 1444 | CASGDAQDTQYF | 23 |
| 185 | CASSYSYEQYF | 26 | 815 | CASSRQGQNTLYF | 27 | 1445 | CASGDAQSQNTLYF | 23 |
| 186 | CASSLVEQYF | 26 | 816 | CASSRQNYAEQFF | 27 | 1446 | CASGDASSGNTLYF | 23 |
| 187 | CASSLDRGNTEVFF | 26 | 817 | CASSRTGENTLYF | 27 | 1447 | CASGDATTSAETLYF | 23 |
| 188 | CASSLLGYEQYF | 26 | 818 | CASSRTGQNTLYF | 27 | 1448 | CASGDAYNSPLYF | 23 |
| 189 | CASSQSSYEQYF | 26 | 819 | CASSSGGQNTLYF | 27 | 1449 | CASGDGGNQDTQYF | 23 |
| 190 | CASSEGDTQYF | 26 | 820 | CASSSGNTLYF | 27 | 1450 | CASGDGNTEVFF | 23 |
| 191 | CASSDRDTQYF | 26 | 821 | CASSSGNYAEQFF | 27 | 1451 | CASGDGTTNTEVFF | 23 |
| 192 | CASSDGDTQYF | 26 | 822 | CASSSGQQNTLYF | 27 | 1452 | CASGDNQAPLF | 23 |
| 193 | CASSFYEQYF | 26 | 823 | CASSSGTTNTEVFF | 27 | 1453 | CASGDNQDTQYF | 23 |
| 194 | CASSYEQYF | 26 | 824 | CASSSQGANTEVFF | 27 | 1454 | CASGDNSAETLYF | 23 |
| 195 | CASSRQSSYEQYF | 26 | 825 | CASSSTANSDYTF | 27 | 1455 | CASGDQNTLYF | 23 |
| 196 | CASSRDREDTQYF | 26 | 826 | CASSWGSQNTLYF | 27 | 1456 | CASGDRANSDYTF | 21 |
| 197 | CASSRYEQYF | 26 | 827 | CASSYGQNTLYF | 27 | 1457 | CASGDRDTEVFF | 23 |
| 198 | CASSPQGTEVFF | 26 | 828 | CGARDSAETLYF | 27 | 1458 | CASGDRGNYAEQFF | 23 |
| 199 | CASSPGQGQDTQYF | 25 | 829 | CSAGGQNTLYF | 27 | 1459 | CASGDRNSDYTF | 23 |
| 200 | CASSGDSYEQYF | 25 | 830 | CASGAETLYF | 26 | 1460 | CASGDSYNSPLYF | 23 |
| 201 | CASGDFYEQYF | 25 | 831 | CASGDADSGNTLYF | 26 | 1461 | CASGDVGSQNTLYF | 23 |
| 202 | CASSQGTQYF | 25 | 832 | CASGDAETLYF | 26 | 1462 | CASGDWGAETLYF | 23 |
| 203 | CASSHQDTQYF | 25 | 833 | CASGDAGDQDTQYF | 26 | 1463 | CASGDWGGYAEQFF | 23 |
| 204 | CASSHYEQYF | 25 | 834 | CASGDAGEQYF | 26 | 1464 | CASGDWGQDTQYF | 23 |
| 205 | CASSVQDTQYF | 25 | 835 | CASGDAGGNTLYF | 26 | 1465 | CASGEQDTQYF | 23 |
| 206 | CASSLRGYEQYF | 25 | 836 | CASGDAGNSDYTF | 26 | 1466 | CASGESQNTLYF | 23 |
| 207 | CASSLEQYEQYF | 25 | 837 | CASGDANSGNTLYF | 26 | 1467 | CASGETANTEVFF | 23 |
| 208 | CASSLEGGEQYF | 25 | 838 | CASGDASQNTLYF | 26 | 1468 | CASGNQDTQYF | 23 |
| 209 | CASSLGGDEQYF | 25 | 839 | CASGDAYAEQFF | 26 | 1469 | CASGTGNYAEQFF | 23 |
| 210 | CASSANDSSYEQYF | 25 | 840 | CASGDNSGNTLYF | 26 | 1470 | CASRDSGNTLYF | 23 |
| 211 | CASSLQGGEQYF | 25 | 841 | CASGDPGNYAEQFF | 26 | 1471 | CASRDSSAETLYF | 23 |
| 212 | CASSLGSYEQYF | 25 | 842 | CASGDRGNTEVFF | 26 | 1472 | CASRSAETLYF | 23 |
| 213 | CASSLGDSSYEQYF | 25 | 843 | CASGDRYEQYF | 26 | 1473 | CASSAGSQNTLYF | 23 |
| 214 | CASSLCIVEQYF | 25 | 844 | CASGDSQNTLYF | 26 | 1474 | CASSANTGQLYF | 23 |
| 215 | CASSFGGQDTQYF | 25 | 845 | CASGDSSGNTLYF | 26 | 1475 | CASSDAETLYF | 23 |
| 216 | CASSLGVQDTQYF | 25 | 846 | CASGDSYAEQFF | 26 | 1476 | CASSDAGAETLYF | 23 |
| 217 | CASSDWGSSYEQYF | 25 | 847 | CASGDYAEQFF | 26 | 1477 | CASSDAGNTEVFF | 23 |
| 218 | CASSLEQDTQYF | 25 | 848 | CASGEGQNTLYF | 26 | 1478 | CASSDASSGNTLYF | 23 |
| 219 | CASSPDRDEQYF | 25 | 849 | CASGESAETLYF | 26 | 1479 | CASSDGSAETLYF | 23 |
| 220 | CASSLRGDTQYF | 25 | 850 | CASGGQANTEVFF | 26 | 1480 | CASSDNERLFF | 23 |
| 221 | CASSLAGGYEQYF | 25 | 851 | CASRDWGNYAEQFF | 26 | 1481 | CASSDNSDYTF | 23 |
| 222 | CASSLGLGYEQYF | 25 | 852 | CASRGAETLYF | 26 | 1482 | CASSDNTGQLYF | 23 |
| 223 | CASSFDAEQFF | 25 | 853 | CASSASQNTLYF | 26 | 1483 | CASSDRANTEVFF | 23 |
| 224 | CASSLREQYF | 25 | 854 | CASSDAGNTLYF | 26 | 1484 | CASSDRDSGNTLYF | 23 |
| 225 | CASSQQGYEQYF | 25 | 855 | CASSDAGSQNTLYF | 26 | 1485 | CASSDRNERLFF | 23 |
| 226 | CASSQGNQDTQYF | 25 | 856 | CASSDASAETYLF | 26 | 1486 | CASSDRSQNTLYF | 23 |
| 227 | CASSRDRGDTQYF | 25 | 857 | CASSDRAETLYF | 26 | 1487 | CASSDSSYNSPLYT | 23 |
| 228 | CASSLAQDTQYF | 25 | 858 | CASSDRDTEVFF | 26 | 1488 | CASSDTGQLYF | 23 |
| 229 | CASSRDRQDTQYF | 25 | 859 | CASSDRNTEVFF | 26 | 1489 | CASSDWGQDTQYF | 23 |
| 230 | CASSDEDTQYF | 25 | 860 | CASSDWDQDTQYF | 26 | 1490 | CASSDWGSQNTLYF | 23 |
| 231 | CASGDNYEQYF | 25 | 861 | CASSDWGNYAEQFF | 26 | 1491 | CASSEQGNTEVFF | 23 |
| 232 | CASSPTGGQDTQYF | 25 | 862 | CASSDWGQNTLYF | 26 | 1492 | CASSESAETLYF | 23 |
| 233 | CASSPYEQYF | 25 | 863 | CASSFGAETLYF | 26 | 1493 | CASSFDTGQLYF | 23 |
| 234 | CASSLNAEQFF | 25 | 864 | CASSFGGAETLYF | 26 | 1494 | CASSFGQNTEVFF | 21 |
| 235 | CASSFGDTQYF | 25 | 865 | CASSFGSAETLYF | 26 | 1495 | CASSFNSAETLYF | 23 |
| 236 | CASSLQSSYEQYF | 25 | 866 | CASSFPSGNTLYF | 26 | 1496 | CASSFQNTEVFF | 23 |
| 237 | CASSPGQDTQYF | 25 | 867 | CASSFQGNTEVFF | 26 | 1497 | CASSFSNERLFF | 23 |
| 238 | CASSPGQGNTEVFF | 25 | 868 | CASSFSGAQDTQYF | 26 | 1498 | CASSFTGGQNTLYF | 23 |
| 239 | CASSLDRGYEQYF | 25 | 869 | CASSGDSQNTLYF | 26 | 1499 | CASSFWGNYAEQFF | 23 |
| 240 | CASSLNERLFF | 25 | 870 | CASSGDSYAEQFF | 26 | 1500 | CASSGDWGNYAEQFP | 23 |
| 241 | CASSLLGGQDTQYF | 25 | 871 | CASSGLGNYAEQFF | 26 | 1501 | CASSGQNTEVFF | 23 |
| 242 | CASSQEDIQYF | 25 | 872 | CASSGQGAETLYF | 26 | 1502 | CASSGTGGQDTQYF | 23 |
| 243 | CASSLGLGQDTQYF | 25 | 873 | CASSGTANTGQLYF | 26 | 1503 | CASSGTNTEVFF | 23 |
| 244 | CASSLGLQDTQYF | 25 | 874 | CASSGTSNSDYTF | 26 | 1504 | CASSHNQDTQYF | 21 |
| 245 | CASSLQGDEQYF | 25 | 875 | CASSLAANTGQLYF | 26 | 1505 | CASSINSAETLYF | 23 |
| 246 | CASSPGLGEDTQYF | 25 | 876 | CASSLAGDTGQLYF | 26 | 1506 | CASSIRGNTEVFF | 23 |
| 247 | CASSLLGQDTQYF | 25 | 877 | CASSLAGNQDTQYF | 26 | 1507 | CASSLAGGQNTLYF | 23 |
| 248 | CASSLDGAEQFF | 25 | 878 | CASSLAGNTEVFF | 26 | 1508 | CASSLAGNSDYTF | 23 |
| 249 | CASSQDRDSDYTF | 25 | 879 | CASSLAGNTGQLYF | 26 | 1509 | CASSLDGNQDTQYF | 23 |
| 250 | CASSLTGEDTQYF | 25 | 880 | CASSLAGSAETLYF | 26 | 1510 | CASSLDGNYAEQFF | 23 |
| 251 | CASSPGNTLYF | 25 | 881 | CASSLDANTEVFF | 26 | 1511 | CASSLDNQAPLF | 23 |
| 252 | CASGDRDEQYT | 25 | 882 | CASSLDGNTLYF | 26 | 1512 | CASSLDSANTEVFF | 23 |
| 253 | CASGDGEQYF | 25 | 883 | CASSLDIYAEQFF | 26 | 1513 | CASSLDSTEVFF | 23 |
| 254 | CASSLDKYEQYF | 24 | 884 | CASSLDNERLFF | 26 | 1514 | CASSLDWGDAEQFF | 23 |
| 255 | CASSRDNSYEQYF | 24 | 885 | CASSLDNNQDTQYF | 26 | 1515 | CASSLEAETLYF | 23 |
| 256 | CASSDAGGSYEQYF | 24 | 886 | CASSLDQAPLF | 26 | 1516 | CASSLEGGNYAEQFF | 23 |
| 257 | CASSGTGDEQYF | 24 | 887 | CASSLDRNTLYF | 26 | 1517 | CASSLEQNTEVFF | 23 |
| 258 | CASSLVGAETLYF | 24 | 888 | CASSLDSGNTLYF | 26 | 1518 | CASSLGAQNTLYF | 23 |
| 259 | CASSLGGEDIQYF | 24 | 889 | CASSLDSNQDTQYF | 26 | 1519 | CASSLGASAETLYF | 23 |
| 260 | CASSLGDSDYTF | 24 | 890 | CASSLDSQDTQYF | 26 | 1520 | CASSLGDNQDTQYF | 23 |
| 261 | CASSLVQDTQYF | 24 | 891 | CASSLDTSQNTLYF | 26 | 1521 | CASSLGDTLYF | 23 |
| 262 | CASSTQDTQYF | 24 | 892 | CASSLDWGNTLYF | 26 | 1522 | CASSLGGAGNTLYF | 23 |
| 263 | CASSESYEQYF | 24 | 893 | CASSLEANTGQLYF | 26 | 1523 | CASSLGGGAETLYF | 23 |
| 264 | CASSSDRDEQYF | 24 | 894 | CASSLEDTGQLYF | 26 | 1524 | CASSLGHYAEQPF | 23 |
| 265 | CASSLEDSYEQYF | 24 | 895 | CASSLEGANSDYTF | 26 | 1525 | CASSLGLGENTLYF | 23 |
| 266 | CASSLQGDTQYF | 24 | 896 | CASSLEGQNTLYF | 26 | 1526 | CASSLGLSAETLYF | 23 |
| 267 | CASSLPGQDTQYF | 24 | 897 | CASSLEGSAETLYF | 26 | 1527 | CASSLGNERLFF | 21 |
| 268 | CASSLGQGYEQYF | 24 | 898 | CASSLENTGQLYF | 26 | 1528 | CASSLGQANTGQLYF | 23 |
| 269 | CASSPTGNSDYTF | 24 | 899 | CASSLESAETLYF | 26 | 1529 | CASSLGQGAGNTLYF | 23 |
| 270 | CASSRLGEDTQYF | 24 | 900 | CASSLGASQNTLYF | 26 | 1530 | CASSLGQGNTGQLYF | 23 |
| 271 | CASSRTGGAETLYF | 24 | 901 | CASSLGDNYAEQFF | 26 | 1531 | CASSLGQGNYAEQFF | 23 |
| 272 | CASSLQGQDTQYF | 24 | 902 | CASSLGDSAETLYF | 26 | 1532 | CASSLGQGQNTLYF | 23 |
| 273 | CASSPGQGYEQYF | 24 | 903 | CASSLGDSGNTLYF | 26 | 1533 | CASSLGQNQAPLF | 23 |
| 274 | CASSPGDTQYF | 24 | 904 | CASSLGTGQLYF | 26 | 1534 | CASSLGQYNSPLYF | 23 |
| 275 | CASSRSSYEQYF | 24 | 905 | CASSLGGSNERLFF | 26 | 1535 | CASSLGRNTEVFF | 23 |
| 276 | CASSLSGDEQYF | 24 | 906 | CASSLGQNTGQLYF | 26 | 1536 | CASSLGSSGNTLYF | 23 |
| 277 | CASSSGYEQYF | 24 | 907 | CASSLGQSAETLYF | 26 | 1537 | CASSLGTGGAETLYF | 23 |
| 278 | CASSQTGGQDTQYF | 24 | 908 | CASSLGSGNTLYF | 26 | 1538 | CASSLGTGNTEVFF | 23 |
| 279 | CASSLTGYEQYF | 24 | 909 | CASSLGTGQLYF | 26 | 1539 | CASSLGTTNEREFF | 23 |
| 280 | CASSLDIYEQYF | 24 | 910 | CASSLGTSAETLYF | 26 | 1540 | CASSLLGNQDTQYF | 23 |
| 281 | CASSLSQDTQYF | 24 | 911 | CASSLGTTNTEVFF | 26 | 1541 | CASSLLGNTGQLYF | 23 |
| 282 | CASSLAGSSYEQYF | 24 | 912 | CASSLGVSNERLFF | 26 | 1542 | CASSLLGSAETLYF | 23 |
| 283 | CASSLGGREQYF | 24 | 913 | CASSLLGDQDTQYF | 26 | 1543 | CASSLLGTSAETLYF | 23 |
| 284 | CASSGTGDTQYF | 24 | 914 | CASSLQGGNTLYF | 26 | 1544 | CASSLNNYAEQFF | 23 |
| 285 | CASSGTEDIQYF | 24 | 915 | CASSLQGNQDTQYF | 26 | 1545 | CASSLNYAEQPF | 23 |
| 286 | CASSLTYEQYF | 24 | 916 | CASSLQISNERLFF | 26 | 1546 | CASSLQENTLYF | 23 |
| 287 | CASSLDDTQYF | 24 | 917 | CASSLQSQNTLYF | 26 | 1547 | CASSLQGANERLFF | 23 |
| 288 | CASSLGTEDTQYF | 24 | 918 | CASSLRAETLYF | 26 | 1548 | CASSLQGDTGQLYF | 23 |
| 289 | CSAEDTQYF | 24 | 919 | CASSLRDNQDTQYF | 26 | 1549 | CASSLQGGAETLYF | 23 |
| 290 | CASSRDIYEQYF | 24 | 920 | CASSLRDTGQLYF | 26 | 1550 | CASSLQGGQNTLYF | 23 |
| 291 | CASSLRAEQFF | 24 | 921 | CASSLRGNTEVFF | 26 | 1551 | CASSLQGGTEVFF | 23 |
| 292 | CASSLLGEDTQYF | 24 | 922 | CASSLRGSGNTLYF | 26 | 1552 | CASSLQGGYAEQFF | 23 |
| 293 | CASSFGAEQFF | 24 | 923 | CASSLSGSAETLYF | 26 | 1553 | CASSLQGNQAPLF | 23 |
| 294 | CASSIQDTQYF | 24 | 924 | CASSLSNERLFF | 26 | 1554 | CASSLQGTNERLFF | 23 |
| 295 | CASSIQYEQYF | 24 | 925 | CASSLSTGQLYF | 26 | 1555 | CASSLRANTEVFF | 23 |
| 296 | CASSEGQYEQYF | 24 | 926 | CASSLTASQNTLYF | 26 | 1556 | CASSLRDNYAEQFF | 23 |
| 297 | CASSDRGYEQYF | 24 | 927 | CASSLTDSGNTLYF | 26 | 1557 | CASSLRDSGNTLYF | 23 |
| 298 | CASGEGEQYF | 24 | 928 | CASSLTENTLYF | 26 | 1558 | CASSLRGGQNTLYF | 23 |
| 299 | CASSDYEQYF | 24 | 929 | CASSLTGANTEVFF | 26 | 1559 | CASSLRGNSDYTF | 23 |
| 300 | CASSDAYEQYF | 24 | 930 | CASSLTGDQDTQYF | 26 | 1560 | CASSLSANSDYTF | 23 |
| 301 | CASGEYEQYF | 24 | 931 | CASSLTGGNTLYF | 26 | 1561 | CASSLSANTEVFF | 23 |
| 302 | CASSLSGNTEVFF | 24 | 932 | CASSLTGGSQNTLYF | 26 | 1562 | CASSLSGTEVFF | 23 |
| 303 | CASSLVGEQYF | 24 | 933 | CASSLTGNTLYF | 26 | 1563 | CASSLSNSGNTLYF | 23 |
| 304 | CASSIGQYEQYF | 24 | 934 | CASSLTGQNTLYF | 26 | 1564 | CASSLTGANSDYTF | 23 |
| 305 | CASSIGDTQYF | 24 | 935 | CASSLTGSAETLYF | 26 | 1565 | CASSLTGDSDYTF | 23 |
| 306 | CASSLDSAEQFF | 24 | 936 | CASSLTNTEVFF | 26 | 1566 | CASSLTGDTEVFF | 23 |
| 307 | CASSQDRNTEVFF | 24 | 937 | CASSLTSSAETLYF | 26 | 1567 | CASSLTGDTGQLYF | 23 |
| 308 | CASSEQDTQYF | 23 | 938 | CASSLVGNYAEQFF | 26 | 1568 | CASSLTGGNYAEQFF | 23 |
| 309 | CASSGQDTQYF | 21 | 939 | CASSLVNQDTQYF | 26 | 1569 | CASSLTGNQDTQYF | 23 |
| 310 | CASSLQGEQYF | 23 | 940 | CASSLVSQNTLYF | 26 | 1570 | CASSLTGYAEQFF | 21 |
| 311 | CASSLDNSYEQYF | 23 | 941 | CASSLWGNYAEQFF | 26 | 1571 | CASSLVGNTGQLYF | 23 |
| 312 | CASGDSSYEQYF | 23 | 942 | CASSPDNSGNTLYF | 26 | 1572 | CASSLVGQNTLYF | 23 |
| 313 | CASGIDASYEQYF | 23 | 943 | CASSPDSQNTLYF | 26 | 1573 | CASSLVTGQLYF | 23 |
| 314 | CASSLTGGQDTQYF | 23 | 944 | CASSPDWGQNTLYF | 26 | 1574 | CASSPDTEVFF | 23 |
| 315 | CASSLEGGQDTQYF | 23 | 945 | CASSPGAETLYF | 26 | 1575 | CASSPGANTGQLYF | 23 |
| 316 | CASSLTGGDTQYF | 23 | 946 | CASSPGGNYAEQFF | 26 | 1576 | CASSPGGSAETLYF | 23 |
| 317 | CASSFTGEDTQYF | 23 | 947 | CASSPGQYAEQFF | 26 | 1577 | CASSPGGYAEQFF | 23 |
| 318 | CASSFRDTQYF | 23 | 948 | CASSPGQYNSPLYF | 26 | 1578 | CASSPGLGQNTLYF | 23 |
| 319 | CASSLEAEQFF | 23 | 949 | CASSPGTSSAETLYF | 26 | 1579 | CASSPGLGSQNTLYF | 23 |
| 320 | CASSLGNYEQYF | 23 | 950 | CASSPGTTNTEVFF | 26 | 1580 | CASSPGNQDTQYF | 21 |
| 321 | CASSRQGDTQYF | 23 | 951 | CASSPGTTSAETLYF | 26 | 1581 | CASSPGNTGQLYF | 23 |
| 322 | CASSSTGGYEQYF | 23 | 952 | CASSPQANTGQLYF | 26 | 1582 | CASSPGQGNERLFF | 23 |
| 323 | CASSLGQSSYEQYF | 23 | 953 | CASSPQGAGNTLYF | 26 | 1583 | CASSPGQKNTLYF | 23 |
| 324 | CASSLGDSYEQYF | 23 | 954 | CASSPQGNTGQLYF | 26 | 1584 | CASSPGQNQAPLF | 23 |
| 325 | CASSWDSQDTQYF | 23 | 955 | CASSPQGSQNTLYF | 26 | 1585 | CASSPGTANERLFF | 23 |
| 326 | CASSPRGQDTQYF | 23 | 956 | CASSPTANTGQLYF | 26 | 1586 | CASSPGTDTEVFE | 23 |
| 327 | CASSTASSYEQYF | 23 | 957 | CASSPTGGNYAEQFF | 26 | 1587 | CASSPNSAETLYF | 23 |
| 328 | CASSRLGYEQYF | 23 | 958 | CASSPTGNTGQLYF | 26 | 1588 | CASSPNSQNTLYF | 23 |
| 329 | CASSLPGGQDTQYF | 23 | 959 | CASSPTGNYAEQFF | 26 | 1589 | CASSPQGANTEVFF | 23 |
| 330 | CASSLEYEQYF | 23 | 960 | CASSPTSAETLYF | 26 | 1590 | CASSPQGNSDYTF | 21 |
| 331 | CASSSGSSYEQYF | 23 | 961 | CASSPTSSQNTLYF | 26 | 1591 | CASSPRGAETLYF | 23 |
| 332 | CASSRGQYEQYF | 23 | 962 | CASSQDSQNTLYF | 26 | 1592 | CASSPSSQNTLYF | 23 |
| 333 | CASSQGEQYF | 23 | 963 | CASSQDWGSQNTLYF | 26 | 1593 | CASSPTGGAETLYF | 23 |
| 334 | CASSLDGDTQYF | 23 | 964 | CASSQGNYAEQFF | 26 | 1594 | CASSPTGSAETLYF | 23 |
| 335 | CASRGQANTEVFF | 23 | 965 | CASSQGSAETLYF | 26 | 1595 | CASSPTINQDTQYF | 23 |
| 336 | CASSPPGQQDTQYF | 23 | 966 | CASSQGSGNTLYF | 26 | 1596 | CASSPTTNTEVFF | 23 |
| 337 | CASSPGSSYEQYF | 23 | 967 | CASSQGTANTGQLYT | 26 | 1597 | CASSPTVNQDTQYF | 23 |
| 338 | CASSRDQDTQYF | 23 | 968 | CASSQNTEVFF | 26 | 1598 | CASSQANTEVFF | 23 |
| 339 | CASSRDFYEQYF | 23 | 969 | CASSQQGANTEVFF | 26 | 1599 | CASSQDGSQNTLYF | 23 |
| 340 | CASSSEDTQYF | 23 | 970 | CASSRDNSAETLYF | 26 | 1600 | CASSQDNSGNTLYF | 23 |
| 341 | CASSRDRYEQYF | 23 | 971 | CASSRDNSGNTLYF | 26 | 1601 | CASSQDRGSAETLYF | 23 |
| 342 | CASSEGSSYEQYF | 21 | 972 | CASSRDRGAEQFF | 26 | 1602 | CASSQDSYNSPLYF | 23 |
| 343 | CASSLGDAEQFF | 23 | 973 | CASSRDRNTGQLYF | 26 | 1603 | CASSQDWGQNTLYF | 23 |
| 344 | CASSQDQDTQYF | 23 | 974 | CASSRDSSGNTLYF | 26 | 1604 | CASSQGANSDYFP | 23 |
| 345 | CASSPGTGQDTQYF | 23 | 975 | CASSRDTEGQLYF | 26 | 1605 | CASSQGANTEVFF | 23 |
| 346 | CASSRTGDQDTQYF | 23 | 976 | CASSRDWGNTGQLYF | 26 | 1606 | CASSQGNTEVFF | 23 |
| 347 | CASSLQGRDTQYF | 21 | 977 | CASSRLGQDTQYF | 26 | 1607 | CASSQGNTLYF | 23 |
| 348 | CASSWTGEDTQYF | 23 | 978 | CASSRNTGQLYF | 26 | 1608 | CASSQGTANSDYTF | 23 |
| 349 | CASSWGYEQYT | 23 | 979 | CASSRQANSDYTF | 26 | 1609 | CASSQQGSAFTLYF | 23 |
| 350 | CASSLRGQDTQYF | 23 | 980 | CASSRQGANTEVFF | 26 | 1610 | CASSQQGTEVFF | 23 |
| 351 | CASSLEGVEQYF | 23 | 981 | CASSRQGANTGQLYF | 26 | 1611 | CASSQTANSDYTF | 23 |
| 352 | CASSFKDTQYF | 21 | 982 | CASSRQNTGQLYF | 26 | 1612 | CASSQTGGQNTLYF | 23 |
| 353 | CASSDEGYEQYF | 23 | 983 | CASSRTDSGNTLYF | 26 | 1613 | CASSRDKNTGQLYF | 23 |
| 354 | CASSDADTQYF | 23 | 984 | CASSRTSQNTLYF | 26 | 1614 | CASSRDNNQAPLF | 23 |
| 355 | CASSPDQDTQYF | 23 | 985 | CASSSDSQNTLYF | 26 | 1615 | CASSRDNNQDTQYF | 23 |
| 356 | CASSPGGQDTQYF | 23 | 986 | CASSSDWGQDTQYF | 26 | 1616 | CASSRDRYAEQFF | 23 |
| 357 | CASSLRQYEQYF | 21 | 987 | CASSSGNTEVFF | 26 | 1617 | CASSRDSSNERLFF | 23 |
| 358 | CASSLVSYEQYF | 23 | 988 | CASSSMANTEVFF | 26 | 1618 | CASSRDTNTEVFF | 21 |
| 359 | CASSSTGDEQYF | 23 | 989 | CASSSGQNTLYF | 26 | 1619 | CASSRDTQDTQYF | 23 |
| 360 | CSADSYEQYF | 23 | 990 | CASSSGQQDTQYF | 26 | 1620 | CASSRDWGDTQYF | 23 |
| 361 | CASGEQYF | 23 | 991 | CASSSGTNTEVFF | 26 | 1621 | CASSRDWGSYEQYF | 23 |
| 362 | CASSPDWGYEQYF | 23 | 992 | CASSSQGNSDYTF | 26 | 1622 | CASSRDWNYAEQFF | 23 |
| 363 | CASSLQGEDTQYF | 23 | 993 | CASSSQGNTEVFF | 26 | 1623 | CASSRDWSAETLYF | 23 |
| 364 | CASSLAGGEQYF | 23 | 994 | CASSSQGNTGQLYF | 26 | 1624 | CASSRDYAEQFF | 23 |
| 365 | CASSLGTGQDTQYF | 23 | 995 | CASSSSGNTLYF | 26 | 1625 | CASSRGNTEVFF | 23 |
| 366 | CASSTGEDIQYF | 23 | 996 | CASSSTANTEVFF | 26 | 1626 | CASSRGQNTEVFF | 23 |
| 367 | CASSPGTEDTQYF | 23 | 997 | CASSSTASQNTLYF | 26 | 1627 | CASSRLGANTGQLYF | 23 |
| 368 | CASSDWGYEQYF | 23 | 998 | CASSSTGNTEVFF | 26 | 1628 | CASSRLGENTLYF | 21 |
| 369 | CASSRDRDTQYF | 23 | 999 | CASSSTSAETLYF | 26 | 1629 | CASSRLGSSAETLYF | 23 |
| 370 | CASSQGYEQYF | 23 | 1000 | CASSSYAEQFF | 26 | 1630 | CASSRQANTGQLYF | 23 |
| 371 | CASSDRYEQYF | 23 | 1001 | CASSTGNTGQYF | 26 | 1631 | CASSRQGETLYF | 23 |
| 372 | CASSYYEQYF | 23 | 1002 | CASSWDSQNTLYF | 26 | 1632 | CASSRQGEVFF | 23 |
| 373 | CASSGQGYEQYF | 23 | 1003 | CASSWDSYAEQFF | 26 | 1633 | CASSRQGYAEQFF | 21 |
| 374 | CASSQEGDTQYF | 23 | 1004 | CASSWGNYAEQFF | 26 | 1634 | CASSRQQNTLYF | 23 |
| 375 | CASSQDWEDTQYF | 23 | 1005 | CSKDSAETLYF | 26 | 1635 | CASSRQSAETLYF | 23 |
| 376 | CASSQDWGSYEQYF | 23 | 1006 | CSSSQGTNERLFF | 26 | 1636 | CASSRQYAEQFF | 23 |
| 377 | CASGDVDTQYF | 23 | 1007 | CASGDADEQYF | 25 | 1637 | CASSRTASQNTLYF | 23 |
| 378 | CASSLGQGDTQYF | 22 | 1008 | CASGDADTGQLYF | 25 | 1638 | CASSRTGGNTLYF | 23 |
| 379 | CASSDDEQYF | 22 | 1009 | CASGDAGAEQFF | 25 | 1639 | CASSRTGNTENTF | 23 |
| 380 | CASSLIGGSYEQYF | 22 | 1010 | CASGDAGANSDYTF | 25 | 1640 | CASSRTGNTLYF | 23 |
| 381 | CASSLGSDYTF | 22 | 1011 | CASGDAGDTGQLYF | 25 | 1641 | CASSRTISNERLFF | 23 |
| 382 | CASSQGAEQRF | 22 | 1012 | CASGDAGGAETLYF | 25 | 1642 | CASSRTISAETLYF | 23 |
| 383 | CASSSGIDTQYF | 22 | 1013 | CASGDAGNQDTQYF | 25 | 1643 | CASSSANTEVFF | 23 |
| 384 | CASSLDRGAEQFF | 22 | 1014 | CASGDAGNTEVFF | 25 | 1644 | CASSSDSSQNTLYF | 23 |
| 385 | CASSSGGYEQYF | 22 | 1015 | CASGDAGNYAEQFF | 25 | 1645 | CASSSDWGQNTLYF | 23 |
| 386 | CASSWDNYEQYF | 22 | 1016 | CASGDAGQDTQYF | 25 | 1646 | CASSSGANTEVFF | 23 |
| 387 | CASSFGDEQYF | 22 | 1017 | CASGDAQSGNTLYF | 25 | 1647 | CASSSGGTEVFF | 23 |
| 388 | CASSRTGQDTQYF | 22 | 1018 | CASGDDQDTQYF | 25 | 1648 | CASSSGQNYAEQFF | 23 |
| 389 | CASSLTGQDTQYF | 22 | 1019 | CASGDGGQNTLYF | 25 | 1649 | CASSSGTANSDYTF | 23 |
| 390 | CASSPGGYEQYF | 22 | 1020 | CASGDNSQNTLYF | 25 | 1650 | CASSSGTTNSDYTF | 23 |
| 391 | CASSPGQGDTQYF | 22 | 1021 | CASGDPSAETLYF | 25 | 1651 | CASSSQGAGNTLYF | 23 |
| 392 | CASSLPGGYEQYF | 22 | 1022 | CASGDPSQNTLYF | 25 | 1652 | CASSSQGYAEQFF | 23 |
| 393 | CASSAQDTQYF | 22 | 1023 | CASGDRGSGNTLYF | 25 | 1653 | CASSSTGDTGQLYF | 23 |
| 394 | CASSPTGGYEQYF | 22 | 1024 | CASGDRGSQNTLYF | 25 | 1654 | CASSSTGNSDYTF | 23 |
| 395 | CASSLTGSSYEQYF | 22 | 1025 | CASGDRNTEVFF | 25 | 1655 | CASSTGGNYAEQFF | 23 |
| 396 | CASSLDRDTQYF | 22 | 1026 | CASGDSNERLFF | 25 | 1656 | CASSTGTANTEVFF | 23 |
| 397 | CASSLGGGEQYF | 22 | 1027 | CASGEGGQNTLYF | 25 | 1657 | CASSVNQDTQYF | 23 |
| 398 | CASSSDRYEQYF | 22 | 1028 | CASGGQGNTEVFF | 25 | 1658 | CASSVSAETLYF | 23 |
| 399 | CASSLDSEQYF | 22 | 1029 | CASGGTANTEVFF | 25 | 1659 | CASSWGDQDTQYF | 23 |
| 400 | CASSLAGDTQYF | 22 | 1030 | CASGSAETLYF | 25 | 1660 | CASSWTDSGNTLYF | 23 |
| 401 | CASSLVGAEQFF | 22 | 1031 | CASRDNYAEQFF | 25 | 1661 | CAWSLGDQDTQYF | 23 |
| 402 | CASSQDAEQFF | 22 | 1032 | CASRDSNYAEQFF | 25 | 1662 | CAWSLGGNYAEQFF | 23 |
| 403 | CASSPTGQDTQYF | 22 | 1033 | CASRDSSGNTLYF | 25 | 1663 | CAWSLGGQDTQYF | 23 |
| 404 | CASSLSGGSYEQYF | 22 | 1034 | CASRDWGSAETLYF | 25 | 1664 | CAWSLGSAETLYF | 23 |
| 405 | CASSDGYEQYF | 22 | 1035 | CASRGQNYAEQFF | 25 | 1665 | CAWSLSAETLYF | 23 |
| 406 | CASSPGLGYEQYF | 22 | 1036 | CASSDAGGQNTLYF | 25 | 1666 | CGARDNYAEQFF | 21 |
| 407 | CASSRQGEDTQYF | 22 | 1037 | CASSDANTEVFF | 25 | 1667 | CGARDRNTGQLYF | 23 |
| 408 | CASSLLGSSYEQYF | 22 | 1038 | CASSDGAETLYF | 25 | 1668 | CGARDSQNTLYF | 23 |
| 409 | CASSLGTQDTQYF | 22 | 1039 | CASSDGGNYAEQFF | 25 | 1669 | CGARDWGSAETLYF | 23 |
| 410 | CASSLSGGYAEQFF | 22 | 1040 | CASSDNSGNTLYF | 25 | 1670 | CSADTEVFF | 23 |
| 411 | CASSLQGSYEQYF | 22 | 1041 | CASSDNSQNTLYF | 25 | 1671 | CASGDAANSDYTF | 22 |
| 412 | CASSLTDTQYF | 22 | 1042 | CASSDNTEVFF | 25 | 1672 | CASGDAGEDTQYF | 22 |
| 413 | CASSFSSYEQYF | 22 | 1043 | CASSDRGAETLYF | 25 | 1673 | CASGDAGGEQYF | 22 |
| 414 | CASSPDRGEQYF | 22 | 1044 | CASSDRNSDYTF | 25 | 1674 | CASGDAGGNQDTQYF | 22 |
| 415 | CASSLGGGQDTQYF | 22 | 1045 | CASSEGQNTLYF | 25 | 1675 | CASGDAGISNERLFF | 22 |
| 416 | CASGDIYEQYF | 22 | 1046 | CASSEGSQNTLYF | 25 | 1676 | CASGDAGQNSDYTF | 22 |
| 417 | CASSRDTYEQYF | 22 | 1047 | CASSENSGNTLYF | 25 | 1677 | CASGDAGVSYEQYF | 22 |
| 418 | CASSSQGDTQYF | 22 | 1048 | CASSENTGQLYF | 25 | 1678 | CASGDARYEQYF | 22 |
| 419 | CSADQDTQYF | 22 | 1049 | CASSETANTEVFF | 25 | 1679 | CASGDAWGQDTQYF | 22 |
| 420 | CASSPLGYEQYF | 22 | 1050 | CASSFGGNYAEQFF | 25 | 1680 | CASGDDRGQNTLYF | 22 |
| 421 | CASSLRDNYEQYF | 22 | 1051 | CASSFGGSAETLYF | 25 | 1681 | CASGDGGNTEVFF | 22 |
| 422 | CASSLAYEQYF | 22 | 1052 | CASSFGTANTEVFF | 25 | 1682 | CASGDGGSQNTLYF | 22 |
| 423 | CASSLGQSYEQYF | 22 | 1053 | CASSFNYAEQFF | 25 | 1683 | CASGDGSAETLYF | 22 |
| 424 | CASSLVDTQYF | 22 | 1054 | CASSGQGNTEVFF | 25 | 1684 | CASGDGTANSDYTF | 22 |
| 425 | CASGEGDTQYF | 22 | 1055 | CASSGQGQNTLYF | 25 | 1685 | CASGDIGNYAEQFF | 22 |
| 426 | CASSRTGVYEQYF | 22 | 1056 | CASSGSAETLYF | 25 | 1686 | CASGDNNERLFF | 22 |
| 427 | CASSQDRDEQYF | 22 | 1057 | CASSGSQNTLYF | 25 | 1687 | CASGDNSDYTT | 22 |
| 428 | CASSEGYEQYF | 22 | 1058 | CASSGTGNSDYTF | 25 | 1688 | CASGDPANSDYFF | 22 |
| 429 | CASGESSYEQYF | 22 | 1059 | CASSGTGQNTLYF | 25 | 1689 | CASGDRDNYAEQFF | 22 |
| 430 | CASSIGNQDTQYF | 22 | 1060 | CASSGTTNTGQLYF | 25 | 1690 | CASGDRGNTGQLYF | 22 |
| 431 | CASSLNSYEQYF | 22 | 1061 | CASSINQDTQYF | 25 | 1691 | CASGDRGQDTQYF | 22 |
| 432 | CASSYAEQFF | 22 | 1062 | CASSLAANTEVFF | 25 | 1692 | CASGDSANTEVFF | 22 |
| 433 | CASSLGTGDQYF | 22 | 1063 | CASSLAGAETLYF | 25 | 1693 | CASGDTSAETLYF | 22 |
| 434 | CSAGQYEQYF | 22 | 1064 | CASSLAGDQDTQYF | 25 | 1694 | CASGEEDTQYF | 22 |
| 435 | CASSQTGYEQYF | 22 | 1065 | CASSLAGENTLYF | 25 | 1695 | CASGETGNTEVFF | 22 |
| 436 | CASSLGLGEDTQYF | 22 | 1066 | CASSLAGQNTLYF | 25 | 1696 | CASGETGNYAEQFF | 22 |
| 437 | CASSQDRYEQYF | 22 | 1067 | CASSLDGGQNTLYF | 25 | 1697 | CASGETLYF | 22 |
| 438 | CASSFGETLYF | 22 | 1068 | CASSLDGNTEVFF | 25 | 1698 | CASGPGQGYAEQFF | 22 |
| 439 | CASSLGTGYEQYF | 22 | 1069 | CASSLDGYAEQFF | 25 | 1699 | CASGSQNTLYF | 22 |
| 440 | CASGAFNQAPLF | 28 | 1070 | CASSLDNTGQLYF | 25 | 1700 | CASGTANTEVFF | 22 |
| 441 | CASGDAEQFF | 28 | 1071 | CASSLDRAGNTLYF | 25 | 1701 | CASGTGNSIDYTF | 22 |
| 442 | CASGDAGGQDTQYF | 28 | 1072 | CASSLDRANSDYTF | 25 | 1702 | CASRDNANTEVFF | 22 |
| 443 | CASGDAGNTLYF | 28 | 1073 | CASSLDRDAEQFF | 25 | 1703 | CASRDNQDTQYF | 22 |
| 444 | CASGDAGQNTLYF | 28 | 1074 | CASSLDRGEVFF | 25 | 1704 | CASRGQGNTEVFF | 22 |
| 445 | CASGDAGSQNTLYF | 28 | 1075 | CASSLDRTEVFF | 25 | 1705 | CASRQGANTEVFF | 22 |
| 446 | CASGDAGYEQYF | 28 | 1076 | CASSLEGANTEVFF | 25 | 1706 | CASRQNTEVFF | 22 |
| 447 | CASGIDGSQNTLYT | 28 | 1077 | CASSLEGDQDTQYF | 25 | 1707 | CASRSQNTLYF | 22 |
| 448 | CASGDNYAEQFF | 28 | 1078 | CASSLEGDSDYTF | 25 | 1708 | CASSADSAETLYF | 22 |
| 449 | CASGDQDTQYF | 28 | 1079 | CASSLEGDTGQLYF | 25 | 1709 | CASSAGTANTEVFF | 22 |
| 450 | CASGDRDTQYF | 28 | 1080 | CASSLEGSSAETLYF | 25 | 1710 | CASSDAGSAETLYF | 22 |
| 451 | CASGDSAETLYF | 28 | 1081 | CASSLEGTSAETLYF | 25 | 1711 | CASSDGGNTEVFF | 22 |
| 452 | CASGDSGNTLYF | 28 | 1082 | CASSLENSDYTF | 25 | 1712 | CASSDGTANSDYTF | 22 |
| 453 | CASGDSSQNTLYF | 28 | 1083 | CASSLESQNTLYF | 25 | 1713 | CASSDNSAETLYF | 22 |
| 454 | CASGDWGSAETLYF | 28 | 1084 | CASSLGGGQNTLYF | 25 | 1714 | CASSDPGQDTQYF | 22 |
| 455 | CASGDWGSQNTLYF | 28 | 1085 | CASSLGGGYAEQFF | 25 | 1715 | CASSDRDQDTQYF | 22 |
| 456 | CASRDSAETLYF | 28 | 1086 | CASSLGGSDYTF | 25 | 1716 | CASSDRETLYF | 22 |
| 457 | CASRPGTANTGQLYF | 28 | 1087 | CASSLGHQDTQYF | 25 | 1717 | CASSDRGQDTQYF | 22 |
| 458 | CASSAETLYF | 28 | 1088 | CASSLGISNERLFF | 25 | 1718 | CASSDRGSAETLYF | 22 |
| 459 | CASSDRGQNTLYF | 28 | 1089 | CASSLGLGAETLYF | 25 | 1719 | CASSDIANTEVFF | 22 |
| 460 | CASSDSAETLYF | 28 | 1090 | CASSLGLGYAEQFF | 25 | 1720 | CASSEDTEVFF | 22 |
| 461 | CASSDSQNTLYF | 28 | 1091 | CASSLGNSAETLYF | 25 | 1721 | CASSEGAGNTLYF | 22 |
| 462 | CASSDSSAETLYF | 28 | 1092 | CASSLGQGTEVFF | 25 | 1722 | CASSFDNSGNTLYF | 22 |
| 463 | CASSDSSQNTLYF | 28 | 1093 | CASSLGSSQNTLYF | 25 | 1723 | CASSEDRNTEVFF | 22 |
| 464 | CASSFDSQNTLYF | 28 | 1094 | CASSLGTANTGQLYF | 25 | 1724 | CASSTGERLFF | 22 |
| 165 | CASSFGQNTLYF | 28 | 1095 | CASSLGTASAETLYF | 25 | 1725 | CASSFLGNYAEQFF | 22 |
| 466 | CASSFGSQNTLYF | 28 | 1096 | CASSLGTSSAETLYF | 25 | 1726 | CASSFNERLFF | 22 |
| 467 | CASSMANTEVFF | 28 | 1097 | CASSLGVNYAEQFF | 25 | 1727 | CASSMANSDYTF | 22 |
| 468 | CASSTSAETLYF | 28 | 1098 | CASSLGVSQNTLYF | 25 | 1728 | CASSFQNYAEQFF | 22 |
| 469 | CASSTSQNTLYF | 28 | 1099 | CASSLLGANTGQLYF | 25 | 1729 | CASSFRTENTEVFF | 22 |
| 470 | CASSGTANSDYTF | 28 | 1100 | CASSLPSAETLYF | 25 | 1730 | CASSTSSGNTLYF | 22 |
| 471 | CASSGTTNSDYTF | 28 | 1101 | CASSLQGANTGQLYF | 25 | 1731 | CASSFTANTEVFF | 22 |
| 472 | CASSHSAETLYF | 28 | 1102 | CASSLQGDQDTQYF | 25 | 1732 | CASSGGAETLYF | 22 |
| 473 | CASSHSQNTLYF | 28 | 1103 | CASSLQGSDYTF | 25 | 1733 | CASSGNTLYF | 22 |
| 474 | CASSLAANSDYTF | 28 | 1104 | CASSLQQDTQYF | 25 | 1734 | CASSGQNTGQLYF | 22 |
| 475 | CASSLAGNYAEQFF | 28 | 1105 | CASSLRQNTEVFF | 25 | 1735 | CASSGTDYAEQFF | 22 |
| 476 | CASSLAGSQNTLYF | 28 | 1106 | CASSLRSAETLYF | 25 | 1736 | CASSGTGGNTLYF | 22 |
| 477 | CASSLANSDYTF | 28 | 1107 | CASSLSGAETLYF | 25 | 1737 | CASSGTGNTLYF | 22 |
| 478 | CASSLANTGQLYF | 28 | 1108 | CASSLSGNYAEQFF | 25 | 1738 | CASSGTSAETLYF | 22 |
| 479 | CASSLASAETLYF | 28 | 1109 | CASSLSGQNTLYF | 25 | 1739 | CASSGTSSAETLYF | 22 |
| 480 | CASSLDERLFF | 28 | 1110 | CASSLSGSGNTLYF | 25 | 1740 | CASSGTTSAETLYF | 22 |
| 481 | CASSLDGSQNTLYF | 28 | 1111 | CASSLSGSQNTLYF | 25 | 1741 | CASSHNYAEQFF | 22 |
| 482 | CASSLDNQDTQYF | 28 | 1112 | CASSLSNTEVFF | 25 | 1742 | CASSIGAETLYF | 22 |
| 483 | CASSLDNSGNTLYF | 28 | 1113 | CASSLTASAETLYF | 25 | 1743 | CASSIGGNYAEQFF | 22 |
| 484 | CASSLDNYAEQFF | 28 | 1114 | CASSLTGDYAEQFF | 25 | 1744 | CASSLAGGAETLYF | 22 |
| 485 | CASSLDRYAEQFF | 28 | 1115 | CASSLTGENTLYF | 25 | 1745 | CASSLAQNTEVFF | 22 |
| 486 | CASSLDSAETLYF | 28 | 1116 | CASSLTGGAETLYF | 25 | 1746 | CASSLAQNTLYF | 22 |
| 487 | CASSLDSDYTF | 28 | 1117 | CASSLTGNTGQLYF | 25 | 1747 | CASSLASSGNTLYF | 22 |
| 488 | CASSLDSQNTLYF | 28 | 1118 | CASSLTSQNTLYF | 25 | 1748 | CASSLDGGSAETLYF | 22 |
| 489 | CASSLDSSAETLYF | 28 | 1119 | CASSLVSAETLYF | 25 | 1749 | CASSLDGNSDYTF | 22 |
| 490 | CASSLDSSGNILYF | 28 | 1120 | CASSNTGQLYF | 25 | 1750 | CASSLDGNTGQLYF | 22 |
| 491 | CASSLDSSQNTLYF | 28 | 1121 | CASSNYAEQFF | 25 | 1751 | CASSLDGSAETLYF | 22 |
| 492 | CASSLDSYAEQFF | 28 | 1122 | CASSPDNYAEQFF | 25 | 1752 | CASSLDKEVFF | 22 |
| 493 | CASSLDTEVFF | 28 | 1123 | CASSPDRNTEVFT | 25 | 1753 | CASSLDNNERLFF | 22 |
| 494 | CASSLDTGQLYF | 28 | 1124 | CASSPDRSQNTLYF | 25 | 1754 | CASSLDNNQAPLF | 22 |
| 495 | CASSLDWGNYAEQFF | 28 | 1125 | CASSPDWGQDTQYF | 25 | 1755 | CASSLDRANTEVFF | 22 |
| 496 | CASSLDWGQDTQYF | 28 | 1126 | CASSPGGAETLYF | 25 | 1756 | CASSLDRGQNTLYF | 22 |
| 497 | CASSLDWGSAETLYF | 28 | 1127 | CASSPGGSQNTLYF | 25 | 1757 | CASSLDRGYAEQFF | 22 |
| 498 | CASSLEANSDYTF | 28 | 1128 | CASSPGNSDYTF | 25 | 1758 | CASSLDSNTGQLYF | 22 |
| 499 | CASSLEDSGNTLYF | 28 | 1129 | CASSPGQANTEVFF | 25 | 1759 | CASSLDSSNERLFF | 22 |
| 500 | CASSIEGAETLYF | 28 | 1130 | CASSPGQGQNTLYF | 25 | 1760 | CASSLDWGAETLYF | 22 |
| 501 | CASSLEGNSDYTF | 28 | 1131 | CASSPGQGTEVFF | 25 | 1761 | CASSLEETLYF | 22 |
| 502 | CASSLEGNTEVFF | 28 | 1132 | CASSPGQNQDTQYF | 25 | 1762 | CASSLEGAQDTQYF | 22 |
| 503 | CASSLEGNTGQLYF | 28 | 1133 | CASSPGQNSDYTF | 25 | 1763 | CASSLEGASQNTLYF | 22 |
| 504 | CASSLEGNTLYF | 28 | 1134 | CASSPGQNTLYF | 25 | 1764 | CASSLEGGAETLYF | 22 |
| 505 | CASSLEGNYAEQFF | 28 | 1135 | CASSPGQQNTLYF | 25 | 1765 | CASSLEGGNTLYF | 22 |
| 506 | CASSLEGSGNTLYF | 28 | 1136 | CASSPGQTEVFF | 25 | 1766 | CASSLEGNSGNTLYF | 22 |
| 507 | CASSLEGSQNTLYF | 28 | 1137 | CASSPGTAFTLYF | 25 | 1767 | CASSLEGRQNTLYF | 22 |
| 508 | CASSLFGYAEQFF | 28 | 1138 | CASSPGTANSDYTF | 25 | 1768 | CASSLEGTGNTLYF | 22 |
| 509 | CASSLESANSDYTF | 28 | 1139 | CASSPGTASAETLYF | 25 | 1769 | CASSLEQGAETLYF | 22 |
| 510 | CASSLGAETLYF | 28 | 1140 | CASSPGTGYAEQFF | 25 | 1770 | CASSLEQNTGQLYF | 22 |
| 511 | CASSLGANSDYTF | 28 | 1141 | CASSPGTNTEVFF | 25 | 1771 | CASSLEQYAEQFF | 22 |
| 512 | CASSLGDQDTQYF | 28 | 1142 | CASSPGTTNERLFF | 25 | 1772 | CASSLGGENTLYF | 22 |
| 513 | CASSLGENTLYF | 28 | 1143 | CASSPLGSQNTLYF | 25 | 1773 | CASSLGGETLYF | 22 |
| 514 | CASSLGERLFF | 28 | 1144 | CASSPLGGYAEQFF | 25 | 1774 | CASSLGGRAETLYF | 22 |
| 515 | CASSLGETLYF | 28 | 1145 | CASSPTGNQDTQYF | 25 | 1775 | CASSLGHTEVFF | 22 |
| 516 | CASSLGGAETLYF | 28 | 1146 | CASSPTTSQNTLYF | 25 | 1776 | CASSLGLGNYAEQFF | 22 |
| 517 | CASSLGGNSDYTF | 28 | 1147 | CASSPTVSNERLFF | 25 | 1777 | CASSLGLGQNTLYF | 22 |
| 518 | CASSLGGNTEVFF | 28 | 1148 | CASSPTVSQNTLYF | 25 | 1778 | CASSLGLNQDTQYF | 22 |
| 519 | CASSLGGNTLYF | 28 | 1149 | CASSQANTGQLYF | 25 | 1779 | CASSLGLNYAEQFF | 22 |
| 520 | CASSLGGSAETLYF | 28 | 1150 | CASSQDSYAEQFF | 25 | 1780 | CASSLGLSQNTLYF | 22 |
| 521 | CASSIGGSQNTLYF | 28 | 1151 | CASSQEGSQNTLYF | 25 | 1781 | CASSLGPNTEVFF | 22 |
| 522 | CASSLGNQDTQYF | 28 | 1152 | CASSQGAETLYF | 25 | 1782 | CASSLGQGNERLFF | 22 |
| 523 | CASSLGNSGNTLYF | 28 | 1153 | CASSQGGQNTLYF | 25 | 1783 | CASSLGQNERLFF | 22 |
| 524 | CASSLGNTEVFF | 28 | 1154 | CASSQGNTGQLYF | 25 | 1784 | CASSLGSQDTQYF | 22 |
| 525 | CASSLGNTGQLYF | 28 | 1155 | CASSQGQNTLYF | 25 | 1785 | CASSLGTAETLYF | 22 |
| 526 | CASSLGNTLYF | 28 | 1156 | CASSQGQNYAEQFF | 25 | 1786 | CASSLGTDYAEQFF | 22 |
| 527 | CASSLGQNSDYTF | 28 | 1157 | CASSQNSGNTLYF | 25 | 1787 | CASSLGTGYAEQFF | 22 |
| 528 | CASSLGQNTLYF | 28 | 1158 | CASSQQGAETLYF | 25 | 1788 | CASSLGTNQDTQYF | 22 |
| 529 | CASSLGQNYAEQFF | 28 | 1159 | CASSQQGNTGQLYF | 25 | 1789 | CASSLGTTSAETLYF | 22 |
| 530 | CASSLGQQNTLYF | 28 | 1160 | CASSQQGSGNTLYF | 25 | 1790 | CASSLGTYAEQFF | 22 |
| 531 | CASSLGQSQNTLYF | 28 | 1161 | CASSRDISQNTLYF | 25 | 1791 | CASSLLGAETLYF | 22 |
| 532 | CASSLGQTEVFF | 28 | 1162 | CASSRDNNYAEQFF | 25 | 1792 | CASSLLGENTLYF | 22 |
| 533 | CASSLGSAETLYF | 28 | 1163 | CASSRDNQAPLF | 25 | 1793 | CASSLLGGQNTLYF | 22 |
| 534 | CASSLGSQNTLYF | 28 | 1164 | CASSRDNTEVFF | 25 | 1794 | CASSLNAETLYF | 22 |
| 535 | CASSLGSSAETLYF | 28 | 1165 | CASSRDRANSDYTF | 25 | 1795 | CASSLNNERLFF | 22 |
| 536 | CASSLGTANSDYTF | 28 | 1166 | CASSRDRNTLYF | 25 | 1796 | CASSLNTLYF | 22 |
| 537 | CASSLGITNSDYTF | 28 | 1167 | CASSRDRNYAEQFF | 25 | 1797 | CASSLPGSQNTLYF | 22 |
| 538 | CASSUGNYAEQFF | 28 | 1168 | CASSRDSANSDYTF | 25 | 1798 | CASSLQGERLFF | 22 |
| 539 | CASSLNQDTQYF | 28 | 1169 | CASSRDTEVFF | 25 | 1799 | CASSLQNSGNTLYF | 22 |
| 540 | CASSLNSAETLYF | 28 | 1170 | CASSRGAETLYF | 25 | 1800 | CASSLQYAEQFF | 22 |
| 541 | CASSLNTGQLYF | 28 | 1171 | CASSRGNQDTQYF | 25 | 1801 | CASSLRANTGQLYF | 22 |
| 542 | CASSLQANSDYTF | 28 | 1172 | CASSRGNYAEQFF | 25 | 1802 | CASSLRDTLYF | 22 |
| 543 | CASSLQANTEVFF | 28 | 1173 | CASSRLGSQNTLYF | 25 | 1803 | CASSLRGYAEQFF | 22 |
| 544 | CASSLQGAETLYF | 28 | 1174 | CASSRNQDTQYF | 25 | 1804 | CASSLRNSGNTLYF | 22 |
| 545 | CASSLQGAGNTLYF | 28 | 1175 | CASSRNTEVFF | 25 | 1805 | CASSLRNTEVFF | 22 |
| 546 | CASSLQGANTEVFF | 28 | 1176 | CASSRQGAGNTLYF | 25 | 1806 | CASSLRQNTLYF | 22 |
| 547 | CASSLQGNSDYTF | 28 | 1177 | CASSRQGDTEVFF | 25 | 1807 | CASSLSGNSDYTF | 22 |
| 548 | CASSLQGNTEVFF | 28 | 1178 | CASSRQSQNTLYF | 25 | 1808 | CASSLSNSDYTF | 22 |
| 549 | CASSLQGNIGQLYF | 28 | 1179 | CASSRTANSDYTF | 25 | 1809 | CASSLSTNSDYTF | 22 |
| 550 | CASSLQGSAETLYF | 28 | 1180 | CASSRTASAETLYF | 25 | 1810 | CASSLSVNQDTQYF | 22 |
| 551 | CASSLQGSGNTLVF | 28 | 1181 | CASSRTGGQNTLVF | 25 | 1811 | CASSLTGSGNTLYF | 22 |
| 552 | CASSLQGSQNTLYF | 28 | 1182 | CASSRTGGYAEQFF | 25 | 1812 | CASSLTGTEVFF | 22 |
| 553 | CASSLQGTEVFF | 28 | 1183 | CASSRTGNSDYTF | 25 | 1813 | CASSLTPNTEVFF | 22 |
| 554 | CASSLQNTLYF | 28 | 1184 | CASSRTGNTGQLYF | 25 | 1814 | CASSLTTNTGQLYF | 22 |
| 555 | CASSLRGSQNTLYF | 28 | 1185 | CASSRTGSQNTLYF | 25 | 1815 | CASSLVANSDYTF | 22 |
| 556 | CASSLSAETLYF | 28 | 1186 | CASSRTNTEVEF | 25 | 1816 | CASSLVGSAETLYF | 22 |
| 557 | CASSLSGNTLYF | 28 | 1187 | CASSRTTNTEVFF | 25 | 1817 | CASSLVNSGNTLYF | 22 |
| 558 | CASSLSGSNYAEQFF | 28 | 1188 | CASSRYAEQFF | 25 | 1818 | CASSLVQNTLYF | 22 |
| 559 | CASSLSQNTLYF | 28 | 1189 | CASSSAANTEVFF | 25 | 1819 | CASSPANTGQLYF | 22 |
| 560 | CASSLSQQNTLYF | 28 | 1190 | CASSSANTGQLYF | 25 | 1820 | CASSPDNSQNTLYF | 22 |
| 561 | CASSLSSQNTLYF | 28 | 1191 | CASSSETLYF | 25 | 1821 | CASSPDTSQNTLYF | 22 |
| 562 | CASSLTANSDYTF | 28 | 1192 | CASSSGAETLYF | 25 | 1822 | CASSPGDTEVFF | 22 |
| 563 | CASSLTANTEVFF | 28 | 1193 | CASSSGGAETLYF | 25 | 1823 | CASSPGHQDTQYF | 22 |
| 564 | CASSLTDYNSPLYF | 28 | 1194 | CASSSGGYAEQFF | 25 | 1824 | CASSPGLGSAETLYF | 22 |
| 565 | CASSLTGGYAEQFF | 28 | 1195 | CASSSGNQDTQYF | 25 | 1825 | CASSPGLSQNTLYF | 22 |
| 566 | CASSLTGNYAEQFF | 28 | 1196 | CASSSGQGAETLYF | 25 | 1826 | CASSPGQGNTLYF | 22 |
| 567 | CASSLTGSQNTLYF | 28 | 1197 | CASSSGQGNTEVFF | 25 | 1827 | CASSPGQISNERLFF | 22 |
| 568 | CASSLTSAETLYF | 28 | 1198 | CASSSGSAETLYF | 25 | 1828 | CASSPGQNNQAPLF | 22 |
| 569 | CASSLWGSQNTLYF | 28 | 1199 | CASSSGTGNTEVFF | 25 | 1829 | CASSPGTGNSDYTF | 22 |
| 570 | CASSPDSAETLYF | 28 | 1200 | CASSSNERLFF | 25 | 1830 | CASSPGTGNTEVFF | 22 |
| 571 | CASSPDSSGNTLYF | 28 | 1201 | CASSSNYAEQFF | 25 | 1831 | CASSPQGDTGQLYF | 22 |
| 572 | CASSPDSSQNTLYF | 28 | 1202 | CASSSQANTEVFF | 25 | 1832 | CASSPQGNYAEQFF | 22 |
| 573 | CASSPDWGENTLYF | 28 | 1203 | CASSSQGSAETLYF | 25 | 1833 | CASSPQNTEVFF | 22 |
| 574 | CASSPDWGNYAEQFF | 28 | 1204 | CASSSTGGYAEQFF | 25 | 1834 | CASSPRDSAETLYF | 22 |
| 575 | CASSPGDQDTQYF | 28 | 1205 | CASSTSQNTLYF | 25 | 1835 | CASSPRLGQDTQYF | 22 |
| 576 | CASSPGGQNTLYF | 28 | 1206 | CASSWDNTEVFF | 25 | 1836 | CASSPSGNTLYF | 22 |
| 577 | CASSPGHERLFF | 28 | 1207 | CASSWGNQDTQYF | 25 | 1837 | CASSPTGNTEVFF | 22 |
| 578 | CASSPGQGNSDYTF | 28 | 1208 | CAWSLGSQNTLYF | 25 | 1838 | CASSPTGYAEQFF | 22 |
| 579 | CASSPGQGYAEQFF | 28 | 1209 | CGARDSNYAEQFF | 25 | 1839 | CASSPTISNERLFF | 22 |
| 580 | CASSPGQNTEVFF | 28 | 1210 | CCARDWGYAEQFF | 25 | 1840 | CASSPTNSGNTLYF | 22 |
| 581 | CASSPGQNYAEQFF | 28 | 1211 | CGARQGYAEQFF | 25 | 1841 | CASSTTNTEVFF | 22 |
| 582 | CASSPGQSQNTLYF | 28 | 1212 | CSAGSQNTLYF | 25 | 1842 | CASSPWGNYAEQFF | 22 |
| 583 | CASSPGSQNTLYF | 28 | 1213 | CSASAETLYF | 25 | 1843 | CASSQDRANTEVFF | 22 |
| 584 | CASSPGTANTEVFF | 28 | 1214 | CSASQNTLYF | 25 | 1844 | CASSQDSNQDTQYF | 22 |
| 585 | CASSPNTGQLYF | 28 | 1215 | CASGDADQDTQYF | 24 | 1845 | CASSQDWDQDTQYF | 22 |
| 586 | CASSPNYAEQFF | 28 | 1216 | CASGDAEQYF | 24 | 1846 | CASSQDWGSAETLYF | 22 |
| 587 | CASSPQGNTEVFF | 28 | 1217 | CASGDAGGNYAEQFF | 24 | 1847 | CASSQEGNQDTQYF | 22 |
| 588 | CASSPSAETLYF | 28 | 1218 | CASGDAGSSYEQYF | 24 | 1848 | CASSQEGNTEVFF | 22 |
| 589 | CASSPSQNTLYF | 28 | 1219 | CASGDANTEVFF | 24 | 1849 | CASSQEGQQDTQYF | 22 |
| 590 | CASSPSSAETLYF | 28 | 1220 | CASGDAPSQNTLYF | 24 | 1850 | CASSQGNSGNTLYF | 22 |
| 591 | CASSPTANSDYTF | 28 | 1221 | CASGDDAETLYF | 24 | 1851 | CASSQGQQNTLYF | 22 |
| 592 | CASSPTASQNTLYF | 28 | 1222 | CASGDGGNYAEQFF | 24 | 1852 | CASSQGTASAETLYF | 22 |
| 593 | CASSPTGAETLYF | 28 | 1223 | CASGDRANTEVFF | 24 | 1853 | CASSQGTISNERLFF | 22 |
| 594 | CASSPTGSQNTLYF | 28 | 1224 | CASGDRDQDTQYF | 24 | 1854 | CASSQNYAEQFF | 22 |
| 595 | CASSPYAEQFF | 28 | 1225 | CASGDRGAETLYF | 74 | 1855 | CASSQQGAGNTLYF | 22 |
| 596 | CASSQDTEVFF | 28 | 1226 | CASGDRNYAEQFF | 24 | 1856 | CASSQTGNTGQLYF | 22 |
| 597 | CASSQDWGQDTQYF | 28 | 1227 | CASGDTNSDYTF | 24 | 1857 | CASSRDINQDTQYF | 22 |
| 598 | CASSQEASNSDYTF | 28 | 1228 | CASGDVEQYF | 24 | 1858 | CASSRDISGNTLYF | 22 |
| 599 | CASSQGLGDILYF | 28 | 1229 | CASGDWDSAETLYF | 24 | 1859 | CASSRDKNTEVFF | 22 |
| 600 | CASSQGQNTEVFF | 28 | 1230 | CASGDWGNTGQLYF | 24 | 1860 | CASSRDNANSDYTF | 22 |
| 601 | CASSQGSQNTLYF | 28 | 1231 | CASGDWGQNTLYF | 24 | 1861 | CASSRDRGNTEVFF | 22 |
| 602 | CASSQNTGQLYF | 28 | 1232 | CASGEGSQNTLYF | 24 | 1862 | CASSRDRGTEVFF | 22 |
| 603 | CASSQQGSQNTLYF | 28 | 1233 | CASGETDSGNTLYF | 24 | 1863 | CASSRDRSQNTLYF | 22 |
| 604 | CASSQSLDNQDTQYF | 28 | 1234 | CASGGTANSDYTF | 24 | 1864 | CASSRDSNTEVFF | 22 |
| 605 | CASSRDIQDTQYF | 28 | 1235 | CASGTTNTEVFF | 24 | 1865 | CASSRDTNYAEQFF | 22 |
| 606 | CASSRDIYAEQFF | 28 | 1236 | CASRDRNYAEQFF | 24 | 1866 | CASSRDWEDTQYF | 22 |
| 607 | CASSRDNNERLFF | 28 | 1237 | CASRNTGQLYF | 24 | 1867 | CASSRDWGAETLYF | 22 |
| 608 | CASSRDNQDTQYF | 28 | 1238 | CASRQGSQNTLYF | 24 | 1868 | CASSRDWGQNTLYF | 22 |
| 609 | CASSRDNYAEQFF | 28 | 1239 | CASRWDNYEQYF | 24 | 1869 | CASSRGDQDTQYF | 22 |
| 610 | CASSRDRDTEVFF | 28 | 1240 | CASSDANSGNTLYF | 24 | 1870 | CASSRGGQNTLYF | 22 |
| 611 | CASSRDSAETLYF | 28 | 1241 | CASSDASQNTLYF | 24 | 1871 | CASSRGQGYAEQFF | 22 |
| 612 | CASSRDSQNTLYF | 28 | 1242 | CASSDDNSGNTLYF | 24 | 1872 | CASSRNYAEQFF | 22 |
| 613 | CASSRDSSAETLYF | 28 | 1243 | CASSDGDIGQLYF | 24 | 1873 | CASSRQGDSDYTF | 22 |
| 614 | CASSRDSTAEQFF | 28 | 1244 | CASSDGGQNTLYF | 24 | 1874 | CASSRQNQAPLF | 22 |
| 615 | CASSRDWEQNTLYF | 28 | 1245 | CASSDNNERLFT | 24 | 1875 | CASSRTAETLYF | 22 |
| 616 | CASSRDWGNYAEQFF | 28 | 1246 | CASSDNNQAPLF | 24 | 1876 | CASSRTGGNTEVFF | 22 |
| 617 | CASSRDWGQDTQYF | 28 | 1247 | CASSDQNTLYF | 74 | 1877 | CASSSDSSGNTLYF | 22 |
| 618 | CASSRGQNTLYF | 28 | 1248 | CASSDRGNTEVFF | 24 | 1878 | CASSSGENTLYF | 22 |
| 619 | CASSRGSAETLYF | 28 | 1249 | CASSDRGSGNTLYF | 24 | 1879 | CASSSGGNYAEQFF | 22 |
| 620 | CASSRQANTEVFF | 28 | 1250 | CASSDRNYAEQFF | 24 | 1880 | CASSSGGSAETLYF | 22 |
| 621 | CASSRQGANSDYTF | 28 | 1251 | CASSDSGNTLYF | 24 | 1881 | CASSSGGSQNTLYF | 22 |
| 622 | CASSRQGNSDYTF | 28 | 1252 | CASSDSNERLFF | 74 | 1882 | CASSSGLGQDTQYF | 22 |
| 623 | CASSRQGNTEVFF | 28 | 1253 | CASSDWGNQDTQYF | 24 | 1883 | CASSSGNTGQLYF | 22 |
| 624 | CASSRQGNTGQLYF | 28 | 1254 | CASSDYAEQFT | 24 | 1884 | CASSSGQKNTLYF | 22 |
| 625 | CASSRQGNYAEQFF | 28 | 1255 | CASSEGAETLYF | 24 | 1885 | CASSSGQSQNTLYF | 22 |
| 626 | CASSRQGSQNTLYF | 28 | 1256 | CASSFAETLYF | 24 | 1886 | CASSSGQYAEQFF | 22 |
| 627 | CASSRQNSDYTF | 28 | 1257 | CASSFDWGNYAEQFF | 24 | 1887 | CASSSGSSAETLYF | 22 |
| 628 | CASSRQNTEVFF | 28 | 1258 | CASSFGGSQNTLYF | 24 | 1888 | CASSSGTGAETLYF | 22 |
| 629 | CASSRSQNTLYF | 28 | 1259 | CASSFNTGQLYF | 24 | 1889 | CASSSLGDQDTQYF | 22 |
| 630 | CASSRVGSDYTF | 28 | 1260 | CASSFRAETLYF | 24 | 1890 | CASSSNSAETLYF | 22 |
| 631 | CASSSAETLYF | 28 | 1261 | CASSFRGSQNTLYF | 24 | 1891 | CASSSNSDYTF | 22 |
| 632 | CASSSDSAETLYF | 28 | 1262 | CASSFRNTEVFF | 74 | 1892 | CASSSNSQNTLYF | 22 |
| 633 | CASSSDWGNYAEQFF | 28 | 1263 | CASSFSGNTLYF | 24 | 1893 | CASSSQGNQDTQYF | 22 |
| 634 | CASSSGDQDTQYF | 28 | 1264 | CASSGDNYAEQFF | 24 | 1894 | CASSSQNTEVFF | 22 |
| 635 | CASSSGQNTEVFF | 28 | 1265 | CASSGDSAETLYF | 24 | 1895 | CASSSQNYAEQFF | 22 |
| 636 | CASSSGSQNTLYF | 28 | 1266 | CASSGDSSGNTLYF | 24 | 1896 | CASSSTGSQNTLYF | 22 |
| 637 | CASSSGTANTEVFF | 28 | 1267 | CASSGQGNSDYTF | 24 | 1897 | CASSSTNSDYTF | |
| 638 | CASSSNTGQLYF | 28 | 1268 | CASSGQSQNTLYF | 74 | 1898 | CASSSWGSQNTLYF | 22 |
| 639 | CASSSQGAETLYF | 28 | 1269 | CASSGTAETLYF | 24 | 1899 | CASSTGNYAEQFF | 22 |
| 640 | CASSSQNTLYF | 28 | 1270 | CASSGTGGAETLYF | 24 | 1900 | CASSTGSAETLYF | 22 |
| 641 | CASSSSAETLYF | 28 | 1271 | CASSGTGGNYAEQFF | 24 | 1901 | CASSWGGQNTLYF | 22 |
| 642 | CASSSSQNTLYF | 28 | 1272 | CASSGTGNYAEQFF | 24 | 1902 | CASSWTANTGQLYF | 22 |
| 643 | CASSTSAETLYF | 28 | 1273 | CASSGTTNTEVFF | 24 | 1903 | CASSYGAETLYF | 22 |
| 644 | CASSWDNYAEQFF | 28 | 1274 | CASSIGQNTEVFF | 24 | 1904 | CASSYGGQNTLYF | 22 |
| 645 | CASSWDSAETLYF | 28 | 1275 | CASSIRDNYAEQFF | 24 | 1905 | CASSYNNQAP | 22 |
| 646 | CASSWGQNTLYF | 28 | 1276 | CASSISAETLYF | 24 | 1906 | CASSYQANTEVFF | 22 |
| 647 | CASSWGSAETLYF | 28 | 1277 | CASSLAGANTEVFF | 24 | 1907 | CASSYRNTENFF | 22 |
| 648 | CASSYSAETLYF | 28 | 1278 | CASSLAGNTLYF | 24 | 1908 | CASSYWGNYAEQFF | 22 |
| 649 | CASSYSQNTLYF | 28 | 1279 | CASSLANTEVFT | 24 | 1909 | CAWSLGGQNTLYF | 22 |
| 650 | CAWSLQGYNSPLYF | 28 | 1280 | CASSLDGAETLYF | 24 | 1910 | CAWSLGNYAEQFF | 22 |
| 651 | CASGDAGAETLYF | 27 | 1281 | CASSLDGQNTLYF | 24 | 1911 | CAWSLGSSAETLYF | 22 |
| 652 | CASGDAGDTQYF | 27 | 1282 | CASSLDGSSAETLYF | 24 | 1912 | CAWSLGYEQYF | 22 |
| 653 | CASGDAGGQNTLYF | 27 | 1283 | CASSLDKNTLYF | 24 | 1913 | CGARDNSGNTLYF | 22 |
| 654 | CASGDAGGYAEQFF | 27 | 1284 | CASSLDRDSDYTF | 24 | 1914 | CGARDSSQNTLYF | 22 |
| 655 | CASGDAGNTGQLYF | 27 | 1285 | CASSLDRNYAEQFF | 24 | 1915 | CGARDSSYNSPLYF | 22 |
| 656 | CASGDAGQNTEVFF | 27 | 1286 | CASSLDSNERLFF | 24 | 1916 | CGARDWGSSYEQYF | 22 |
| 657 | CASGDAGSAETLYF | 27 | 1287 | CASSLDSNYAEQFF | 24 | 1917 | CGARQGNTEVFF | 22 |
| 658 | CASGDANSDYTF | 27 | 1288 | CASSLDSYNSPLYF | 24 | 1918 | CGARTVSNERLFF | 22 |
| 659 | CASGDARDTQYF | 27 | 1289 | CASSLDTNTEVFF | 24 | 1919 | CSADAEQFF | 22 |
| 660 | CASGDASAETLYF | 27 | 1290 | CASSLEGDAEQFF | 74 | 1920 | CSAGNSDYTF | 22 |
| 661 | CASGDNTEVFF | 27 | 1291 | CASSLEGGYAEQFF | 24 | 1921 | CSAGQNTEVFF | 22 |
| 662 | CASGDNYNSPLYF | 27 | 1292 | CASSLEGQQDTQYF | 24 | 1922 | CSAGSAETLYF | 22 |
| 663 | CASGDSSAETLYF | 27 | 1293 | CASSLEGTEVFF | 24 | 1923 | CASSQSRYEQYF | |
| 664 | CASGDWGNQDTQYF | 27 | 1294 | CASSLENSGNTLYF | 24 | | | |

Each possibility represents a separate embodiment of the present invention.

According to some embodiments of the present invention, the CDR3 sequence is selected from the group consisting of the sequences in Table 8.

According to some embodiments of the present invention, the peptide amino acid sequence is selected from the group consisting of SEQ ID NOs of Table 8.

TABLE 8

| | | sharing |
|---|---|---|
| SEQ | | level |
| ID | | in |
| NO: | sequence | mice |
| 2 | CASSGTGQDTQYF | 27 |
| 3 | CASSGTGEDIQYF | 25 |
| 4 | CASSGLGEDTQYF | 24 |
| 5 | CASGGYEQYF | 23 |
| 6 | CASSPGGSYEQYF | 20 |
| 7 | CASSSRYEQYF | 17 |
| 8 | CASSGTGKDTQYF | 17 |
| 9 | CASSFGVSYEQYF | 12 |
| 10 | CASSRGSYEQYF | 11 |
| 11 | CASSPGTGVEQYF | 9 |
| 12 | CASSFGTGYEQYF | 9 |
| 13 | CASSGGAYEQYF | 6 |
| 14 | CASSLGVGDTQYF | 5 |
| 15 | CASTGTGQDTQYF | 5 |
| 16 | CASSGRGQDTQYF | 4 |
| 17 | CASGGAYEQYF | 2 |
| 18 | CASSFVGSYEQYF | 1 |
| 19 | CASSRRPYEQYF | 1 |
| 20 | CASSLGGQNTLYF | 28 |
| 21 | CASSLGNSDYTF | 28 |
| 22 | CASSSANSDYTF | 27 |
| 23 | CASSSGNSDYTF | 27 |
| 24 | CASSGTANTEVFF | 26 |
| 25 | CASSGQGNYAEQFF | 26 |
| 26 | CASGDWGYEQYF | 25 |
| 27 | CASGDAGGSYEQYF | 25 |
| 28 | CASSDGANTEVFF | 24 |
| 29 | CASSHSGNTLYF | 23 |
| 30 | CASSGTDQDTQYF | 23 |
| 31 | CASSPGQSNERLFF | 23 |
| 32 | CASSRTANTGQLYF | 23 |
| 33 | CASSDSANTEVFF | 22 |
| 34 | CASSLEGDTEVFF | 22 |
| 35 | CASSLEGEDTQYF | 28 |
| 36 | CASSPGQQDTQYF | 28 |
| 37 | CASSFQDTQYF | 28 |
| 38 | CASSRQQDTQYF | 28 |
| 39 | CASSRDSQDTQYF | 28 |
| 40 | CASSLQGYEQYF | 28 |
| 41 | CASSDSSYEQYF | 28 |
| 42 | CASSLGSSYEQYF | 28 |
| 43 | CASGDGDTQYF | 28 |
| 44 | CASGDYEQYF | 28 |
| 45 | CASSLEDTQYF | 28 |
| 46 | CASSLEGDEQYF | 28 |
| 47 | CASSLGYEQYF | 28 |
| 48 | CASSLGQYEQYF | 28 |
| 49 | CASSVDGSYEQYF | 28 |
| 50 | CASSPQDTQYF | 28 |
| 51 | CASSLDNYEQYF | 28 |
| 52 | CASSLEGYEQYF | 28 |
| 53 | CASSLDEQYF | 28 |
| 54 | CASSLEGNQDTQYF | 28 |
| 55 | CASSLEGDTQYF | 28 |
| 56 | CASSLDYEQYF | 28 |
| 57 | CASSLGDTQYF | 28 |
| 58 | CASSLGEQYF | 28 |
| 59 | CASSQDTQYF | 28 |
| 60 | CASSLDRYEQYF | 28 |
| 61 | CASSGTGGYEQYF | 28 |
| 62 | CASSSYEQYF | 28 |
| 63 | CASSSGQYEQYF | 28 |
| 64 | CASSLLGGAEQFF | 28 |
| 65 | CASSLDRDEQYF | 28 |
| 66 | CASSLDQDTQYF | 28 |
| 67 | CASSLEGSSYEQYF | 28 |
| 68 | CASSLGGYEQYF | 28 |
| 69 | CASSLGAEQFF | 28 |
| 70 | CASSLSGYEQYF | 28 |
| 71 | CASSSSYEQYF | 28 |
| 72 | CASSLGQDTQYF | 28 |
| 73 | CASSLGGQDTQYF | 28 |
| 74 | CASSFNQDTQYF | 28 |
| 75 | CASGQDTQYF | 28 |
| 76 | CASSRDWGYEQYF | 28 |
| 77 | CASSLDSYEQYF | 28 |
| 78 | CASSRQYEQYF | 28 |
| 79 | CASSLKDTQYF | 28 |
| 80 | CASSLGQNTEVFF | 28 |
| 81 | CASSFGTGDEQYF | 28 |
| 82 | CASSLGEDTQYF | 28 |
| 83 | CASSRQNQDTQYF | 28 |
| 84 | CASSPSSYEQYF | 28 |
| 85 | CASSPDSYEQYF | 28 |
| 86 | CASSRDNYEQYF | 28 |
| 87 | CASSRDSYEQYF | 28 |
| 88 | CASSLDRVEQYF | 28 |
| 89 | CASSLYAEQFF | 28 |
| 90 | CASSLDAEQFF | 28 |
| 91 | CASSDSYEQYF | 28 |
| 92 | CASSFGTEVFF | 28 |
| 93 | CASSPDNYEQYF | 28 |
| 94 | CASSPGQYEQYF | 28 |
| 95 | CASSLQDTQYF | 28 |
| 96 | CASSLRDTQYF | 28 |
| 97 | CASSLGDEQYF | 28 |
| 98 | CASSLVAEQFF | 28 |
| 99 | CASSFSYEQYF | 28 |
| 100 | CASSLSYEQYF | 28 |
| 101 | CASSDQDTQYF | 28 |
| 102 | CASSDAGDTQYF | 28 |
| 103 | CASGDSYEQYF | 28 |
| 104 | CASSLDTQYF | 28 |
| 105 | CASSFYAEQFF | 28 |
| 106 | CASGDEQYF | 28 |
| 107 | CASGDAYEQYF | 28 |
| 108 | CASSLYEQYF | 28 |
| 109 | CASSRDSSYEQYF | 28 |
| 110 | CASSIRDTQYF | 28 |
| 111 | CASSRTGYEQYF | 27 |
| 112 | CASSDAGYEQYF | 27 |
| 113 | CASSQQDTQYF | 27 |
| 114 | CASSGQQDTQYF | 27 |
| 115 | CASGEDTQYF | 27 |
| 116 | CASSRQDTQYF | 27 |
| 117 | CASSLGQQDTQYF | 27 |
| 118 | CASSGGEQYF | 27 |
| 119 | CASSYQDTQYF | 27 |
| 120 | CASSLQYEQYF | 27 |
| 121 | CASSSQDTQYF | 27 |
| 122 | CASSQDRDTEVFF | 27 |
| 123 | CASSNQDTQYF | 27 |
| 124 | CASSSSSYEQYF | 27 |
| 125 | CASSLEGAEQFF | 27 |
| 126 | CASSFGQYEQYF | 27 |
| 127 | CASSLTGDEQYF | 27 |
| 128 | CASSRQGYEQYF | 27 |
| 129 | CASSSTGYEQYF | 27 |
| 130 | CASSLDGYEQYF | 27 |
| 131 | CASSSNQDTQYF | 27 |
| 132 | CASSPTGDEQYF | 27 |
| 133 | CASSLSAEQFF | 27 |
| 134 | CASSLDRGEQYF | 27 |
| 135 | CASSLGYAEQFF | 27 |
| 136 | CASSSQGYEQYF | 27 |
| 137 | CASSPSYEQYF | 27 |
| 138 | CASSPDRYEQYF | 27 |
| 139 | CASSPNQDTQYF | 27 |
| 140 | CASSLSSYEQYF | 27 |
| 141 | CASSLAGYEQYF | 27 |
| 142 | CASSLTGGYEQYF | 27 |
| 143 | CASSLDTYEQYF | 27 |
| 144 | CASSLGGSSYEQYF | 27 |
| 145 | CASGYEQYF | 27 |
| 146 | CASGDADTQYF | 27 |
| 147 | CASSLVGYEQYF | 27 |
| 148 | CASGDEDTQYF | 27 |
| 149 | CASSQGQYEQYF | 27 |
| 150 | CASSLDSSYEQYF | 27 |
| 151 | CASSPTGYEQYF | 27 |
| 152 | CASSQYEQYF | 27 |
| 153 | CASSRQGQDTQYF | 26 |
| 154 | CASSLQGAEQFF | 26 |
| 155 | CASSDNYEQYF | 26 |
| 156 | CASSDRGDTQYF | 26 |
| 157 | CASSDAEQFF | 26 |
| 158 | CASSGQYEQYF | 26 |
| 159 | CASSSGGQDTQYF | 26 |
| 160 | CASSLGGAEQFF | 26 |
| 161 | CASSPDAEQFF | 26 |
| 162 | CASSLGGSYEQYF | 26 |
| 163 | CASSIGDEQYF | 26 |
| 164 | CASSESGGYEQYF | 26 |
| 165 | CASSYNQDTQYF | 26 |
| 166 | CASSLEGEQYF | 26 |
| 167 | CASSRDRGYEQYF | 26 |
| 168 | CASSLGGEQYF | 26 |
| 169 | CASSLEEQYF | 26 |
| 170 | CASGDDEQYF | 26 |
| 171 | CASSRLPSYEQYF | 26 |
| 172 | CASSLAGGQDTQYF | 26 |
| 173 | CASSRTGGQDTQYF | 26 |
| 174 | CASSLEGQDTQYF | 26 |
| 175 | CASSLVGDEQYF | 26 |
| 176 | CASSPQGYEQYF | 26 |
| 177 | CASSSDSYEQYF | 26 |
| 178 | CASSRTGEDTQYF | 26 |
| 179 | CASSRTGGYEQYF | 26 |
| 180 | CASSLQGSSYEQYF | 26 |
| 181 | CASSLTGNTEVFF | 26 |
| 182 | CASSLGGDTQYF | 26 |
| 183 | CASSLDWGYEQYF | 26 |
| 184 | CASSPGQSSYEQYF | 26 |
| 185 | CASSYSYEQYF | 26 |
| 186 | CASSLVEQYF | 26 |
| 187 | CASSLDRGNTEVFF | 26 |
| 188 | CASSLLGYEQYF | 26 |
| 189 | CASSQSSYEQYF | 26 |
| 190 | CASSEGDTQYF | 26 |
| 191 | CASSDRDTQYF | 26 |
| 192 | CASSDGDTQYF | 26 |
| 193 | CASSFYEQYF | 26 |
| 194 | CASSYEQYF | 26 |
| 195 | CASSRQSSYEQYF | 26 |
| 196 | CASSRDREDTQYF | 26 |
| 197 | CASSRYEQYF | 26 |
| 198 | CASSPQGTEVFF | 26 |
| 199 | CASSPGQGQDTQYF | 25 |
| 200 | CASSGDSYEQYF | 25 |
| 201 | CASGDFYEQYF | 25 |
| 202 | CASSQGTQYF | 25 |
| 203 | CASSHQDTQYF | 25 |
| 204 | CASSHYEQYF | 25 |
| 205 | CASSVQDTQYF | 25 |
| 206 | CASSLRGYEQYF | 25 |
| 207 | CASSLEQYEQYF | 25 |
| 208 | CASSLEGGEQYF | 25 |
| 209 | CASSLGGDEQYF | 25 |
| 210 | CASSWDSSYEQYF | 25 |
| 211 | CASSLQGGEQYF | 25 |
| 212 | CASSLGSYEQYF | 25 |
| 213 | CASSLGDSSYEQYF | 25 |
| 214 | CASSLGVEQYF | 25 |
| 215 | CASSFGGQDTQYF | 25 |
| 216 | CASSLGVQDTQYF | 25 |
| 217 | CASSDWGSSYEQYF | 25 |
| 218 | CASSLEQDTQYF | 25 |
| 219 | CASSPDRDEQYF | 25 |
| 220 | CASSLRGDTQYF | 25 |
| 221 | CASSLAGGYEQYF | 25 |
| 222 | CASSLGLGYEQYF | 25 |
| 223 | CASSFDAEQFF | 25 |
| 224 | CASSLREQYf | 25 |
| 225 | CASSQQGYEQYF | 25 |
| 226 | CASSQGNQDTQYF | 25 |
| 227 | CASSRDRGDTQYF | 25 |
| 228 | CASSLAQDTQYF | 25 |
| 229 | CASSRDRQDTQYF | 25 |
| 230 | CASSDEDTQYF | 25 |
| 231 | CASGDNYEQYF | 25 |
| 232 | CASSPTGGQDTQYF | 25 |
| 233 | CASSPYEQYF | 25 |
| 234 | CASSLNAEQFF | 25 |
| 235 | CASSFGDTQYF | 25 |
| 236 | CASSLQSSYEQYF | 25 |
| 237 | CASSPGQDTQYF | 25 |
| 238 | CASSPGQGNTEVFF | 25 |
| 239 | CASSLDRGYEQYF | 25 |
| 240 | CASSILNERLFF | 25 |
| 241 | CASSLLGGQDTQYF | 25 |
| 242 | CASSQEDTQYF | 25 |
| 243 | CASSLGLGQDTQYF | 25 |
| 244 | CASSLGLQDTQYF | 25 |
| 245 | CASSLQGDEQYF | 25 |
| 246 | CASSPGLGEDTQYF | 25 |
| 247 | CASSLLGQDTQYF | 25 |
| 248 | CASSLDGAEQFF | 25 |
| 249 | CASSQDRDSDYTF | 25 |
| 250 | CASSLTGEDTQYF | 25 |
| 251 | CASSPGNTLYF | 25 |
| 252 | CASGDRDEQYF | 25 |
| 253 | CASGDGEQYF | 25 |
| 254 | CASSLDKYEQYF | 24 |
| 255 | CASSRDNSYEQYF | 24 |
| 256 | CASSDAGGSYEQYF | 24 |
| 257 | CASSGTGDEQYF | 24 |
| 258 | CASSLVGAETLYF | 24 |
| 259 | CASSLGGEDTQYF | 24 |
| 260 | CASSLGDSDYTF | 24 |
| 261 | CASSLVQDTQYF | 24 |
| 262 | CASSTQDTQYF | 24 |
| 263 | CASSHSYEQYF | 24 |
| 264 | CASSSDRDEQYF | 24 |
| 265 | CASSLEDSYEQYF | 24 |
| 266 | CASSLQGDTQYF | 24 |
| 267 | CASSLPGQDTQYF | 24 |
| 268 | CASSLGQGYEQYF | 24 |
| 269 | CASSPTGNSDYTF | 24 |
| 270 | CASSRLGEDTQYF | 24 |
| 271 | CASSRTGGAETLYF | 24 |
| 272 | CASSLQGQDTQYF | 24 |
| 273 | CASSPGQGYEQYF | 24 |
| 274 | CASSPGDTQYF | 24 |
| 275 | CASSRSSYEQYF | 24 |
| 276 | CASSLSGDEQYF | 24 |
| 277 | CASSSGYEQYF | 24 |
| 278 | CASSQTGGQDTQYF | 24 |
| 279 | CASSLTGYEQYF | 24 |
| 280 | CASSLDIYEQYF | 24 |
| 281 | CASSLSQDTQYF | 24 |
| 282 | CASSLAGSSYEQYF | 24 |
| 283 | CASSLGGREQYF | 24 |
| 284 | CASSGTGDTQYF | 24 |
| 285 | CASSGTEDTQYF | 24 |
| 286 | CASSLTYEQYF | 24 |
| 287 | CASSLDDTQYF | 24 |
| 288 | CASSLGTEDTQYF | 24 |
| 289 | CSAFDTQYF | 24 |
| 290 | CASSRDIYEQYF | 24 |
| 291 | CASSLRAEQFF | 24 |
| 292 | CASSLLGEDTQYF | 24 |
| 293 | CASSFGAEQFF | 24 |
| 294 | CASSIQDTQYF | 24 |
| 295 | CASSIQYEQYF | 24 |
| 296 | CASSEGQYEQYF | 24 |
| 297 | CASSDRGYEQYF | 24 |
| 298 | CASGEGEQYF | 24 |
| 299 | CASSDYEQYF | 24 |
| 300 | CASSDAYEQYF | 24 |
| 301 | CASGEYEQYF | 24 |
| 302 | CASSLSGNTEVFF | 24 |
| 303 | CASSLVGEQYF | 24 |
| 304 | CASSIGQYEQYF | 24 |
| 305 | CASSIGDTQYF | 24 |
| 306 | CASSLDSAEQFF | 24 |
| 307 | CASSQDRNTEVFF | 24 |
| 308 | CASSEQDTQYF | 23 |
| 309 | CASSGQDTQYF | 23 |
| 310 | CASSLQGEQYF | 23 |
| 311 | CASSLDNSYEQYF | 23 |
| 312 | CASGDSSYEQYF | 23 |
| 313 | CASGDASYEQYF | 23 |
| 314 | CASSLTGGQDTQYF | 23 |
| 315 | CASSLEGGQDTQYF | 23 |
| 316 | CASSLIGGDTQYF | 23 |
| 317 | CASSFTGEDTQYF | 23 |
| 318 | CASSFRDTQYF | 23 |
| 319 | CASSLEAEQFF | 23 |
| 320 | CASSLGNYEQYF | 23 |
| 321 | CASSRQGDTQYF | 23 |
| 322 | CASSSTGGYEQYF | 23 |
| 323 | CASSLGQSSYEQYF | 23 |
| 324 | CASSLGDSYEQYF | 21 |
| 325 | CASSWDSQDTQYF | 23 |
| 326 | CASSPRGQDTQYF | 23 |
| 327 | CASSLRSSYEQYF | 23 |
| 328 | CASSRLGYEQYF | 23 |
| 329 | CASSLPGGQDTQYF | 23 |
| 330 | CASSLEYEQYF | 23 |
| 331 | CASSSGSSYEQYF | 23 |
| 332 | CASSRGQYEQYF | 23 |
| 333 | CASSQGEQYF | 23 |
| 334 | CASSLDGDTQYF | 21 |
| 335 | CASRGQANTEVFF | 23 |
| 336 | CASSPPGQQDTQYF | 23 |
| 337 | CASSPGSSYEQYF | 23 |
| 338 | CASSRDQDTQYF | 23 |
| 339 | CASSRDFYEQYF | 21 |
| 340 | CASSSEDTQYF | 23 |
| 341 | CASSRDRYEQYF | 23 |
| 342 | CASSEGSSYEQYF | 23 |
| 343 | CASSLGDAEQFF | 23 |
| 344 | CASSQDQDTQYF | 21 |
| 345 | CASSPGTGQDTQYF | 23 |
| 346 | CASSRTGDQDTQYF | 23 |
| 347 | CASSLQGRDTQYF | 23 |
| 348 | CASSWTGEDTQYF | 23 |
| 349 | CASSWGYEQYF | 23 |
| 350 | CASSLRGQDTQYF | 23 |
| 351 | CASSLEGVEQYF | 23 |
| 352 | CASSFKDTQYF | 23 |
| 353 | CASSDEGYEQYF | 23 |
| 354 | CASSDADTQYF | 23 |
| 355 | CASSPDQDTQYF | 23 |
| 356 | CASSPGGQDTQYF | 23 |
| 357 | CASSLRQYEQYF | 23 |
| 358 | CASSLVSYEQYF | 23 |
| 359 | CASSSTGDEQYF | 23 |
| 360 | CSADSYEQYF | 23 |
| 361 | CASGEQYF | 23 |
| 362 | CASSPDWGYEQYF | 23 |
| 363 | CASSLQGEDTQYF | 23 |
| 364 | CASSLAGGEQYF | 23 |
| 365 | CASSLGTGQDTQYF | 23 |
| 366 | CASSTGEDTQYF | 23 |
| 367 | CASSPGTEDTQYF | 21 |
| 368 | CASSDWGYEQYF | 23 |
| 369 | CASSRDRDTQYF | 23 |
| 370 | CASSQGYEQYF | 23 |
| 371 | CASSDRYEQYF | 23 |
| 372 | CASSYYEQYF | 21 |
| 373 | CASSGQGYEQYF | 23 |
| 374 | CASSQEGDTQYF | 23 |
| 375 | CASSQDWEDTQYF | 23 |
| 376 | CASSQDWGSYEQYF | 23 |
| 377 | CASGDVDTQYF | 21 |
| 378 | CASSLGQGDTQYF | 22 |
| 379 | CASSDDEQYF | 22 |
| 380 | CASSLTGGSYEQYF | 22 |
| 381 | CASSLGSDYTF | 22 |
| 382 | CASSQGAEQFF | 22 |
| 383 | CASSSGDTQYF | 22 |
| 384 | CASSLDRGAEQFF | 22 |
| 385 | CASSSGGYEQYF | 22 |
| 386 | CASSWDNYEQYT | 22 |
| 387 | CASSFGDEQYF | 22 |
| 388 | CASSRTGQDTQYF | 22 |
| 389 | CASSLTGQDTQYF | 22 |
| 390 | CASSPGGYEQYF | 22 |
| 391 | CASSPGQGDTQYF | 22 |
| 392 | CASSLPGGYEQYF | 22 |
| 393 | CASSAQDTQYF | 22 |
| 394 | CASSPTGGYEQYF | 22 |
| 395 | CASSLTGSSYEQYF | 22 |
| 396 | CASSLDRDTQYF | 22 |
| 397 | CASSLGGGEQYF | 22 |
| 398 | CASSSDRYEQYF | 22 |
| 399 | CASSLDSEQYF | 22 |
| 400 | CASSLAGDTQYF | 22 |
| 401 | CASSLVGAEQFF | 22 |
| 402 | CASSQDAEQFF | 22 |
| 403 | CASSPTGQDTQYF | 22 |
| 404 | CASSLSGGSYEQYF | 22 |
| 405 | CASSDGYEQYF | 22 |
| 406 | CASSPGLGYEQYF | 22 |
| 407 | CASSRQGEDTQYF | 22 |
| 408 | CASSLLGSSYEQYF | 22 |
| 409 | CASSLGTQDTQYF | 22 |
| 410 | CASSLSGGYAEQFF | 22 |
| 411 | CASSLQGSYEQYF | 22 |
| 412 | CASSLTDTQYF | 22 |
| 413 | CASSTSSYEQYF | 22 |
| 414 | CASSPDRGEQYF | 22 |
| 415 | CASSLGGGQDTQYF | 22 |
| 416 | CASGDIYEQYF | 22 |
| 417 | CASSRDTYEQYF | 22 |
| 418 | CASSSQGDTQYF | 22 |
| 419 | CSADQDTQYF | 22 |
| 420 | CASSPLGYEQYF | 22 |
| 421 | CASSLRDNYEQYF | 22 |
| 422 | CASSLAYEQYF | 22 |
| 423 | CASSLGQSYEQYF | 22 |
| 424 | CASSLVDTQYF | 22 |
| 425 | CASGEGDTQYF | 22 |
| 426 | CASSRTGVYEQYF | 22 |
| 427 | CASSQDRDEQYF | 22 |
| 428 | CASSEGYEQYF | 22 |
| 429 | CASGESSYEQYF | 22 |
| 430 | CASSTGNQDTQYF | 22 |
| 431 | CASSLNSYEQYF | 22 |
| 432 | CASSYAEQFF | 22 |
| 433 | CASSLGTGDTQYF | 22 |
| 434 | CSAGQYEQYF | 22 |
| 435 | CASSQTGYEQYF | 22 |
| 436 | CASSLGLGEDTQYF | 22 |
| 437 | CASSQDRYEQYF | 22 |
| 438 | CASSFGETLYF | 22 |
| 439 | CASSLGTGYEQYF | 22 |
| 440 | CASGAFNQAPLF | 28 |
| 441 | CASGDAEQFF | 28 |
| 442 | CASGDAGGQDTQYF | 28 |
| 443 | CASGDAGNTLYF | 28 |
| 444 | CASGDAGQNTLYF | 28 |
| 445 | CASGDAGSQNTLYF | 28 |
| 446 | CASGDAGYEQYF | 28 |
| 447 | CASGDGSQNTLYF | 28 |
| 448 | CASGDNYAEQFF | 28 |
| 449 | CASGDQDTQYF | 28 |
| 450 | CASGDRDIQYF | 28 |
| 451 | CASGDSAETLYF | 28 |
| 452 | CASGDSGNTLYF | 28 |
| 453 | CASGDSSQNTLYF | 28 |
| 454 | CASGDWGSAETLYF | 28 |
| 455 | CASGDWGSQNTLYF | 28 |
| 456 | CASRDSAETLYF | 28 |
| 457 | CASRPGTANTGQLYF | 28 |
| 458 | CASSAETLYF | 28 |
| 459 | CASSDRGQNTLYF | 28 |
| 460 | CASSDSAETLYF | 28 |
| 461 | CASSDSQNTLYF | 28 |
| 462 | CASSDSSAETLYF | 28 |
| 463 | CASSDSSQNTLYF | 28 |
| 464 | CASSFDSQNTLYF | 28 |
| 465 | CASSFGQNTLYF | 28 |
| 466 | CASSRISQNTLYF | 28 |
| 467 | CASSFQANTEVFF | 28 |
| 468 | CASSFSAETLYF | 28 |
| 469 | CASSFSQNTLYF | 28 |
| 470 | CASSGTANSDYTF | 28 |
| 471 | CASSGTTNSDYTF | 28 |
| 472 | CASSHSAETLYF | 28 |
| 473 | CASSHSQNTLYF | 28 |
| 474 | CASSLAANSDYTF | 28 |
| 475 | CASSLAGNYAEQFF | 28 |
| 476 | CASSLAGSQNTLYF | 28 |
| 477 | CASSLANSDYTF | 28 |
| 478 | CASSLANTGQLYF | 28 |
| 479 | CASSIASAETLYF | 28 |
| 480 | CASSLDERLFF | 28 |
| 481 | CASSLDGSQNTLYF | 28 |
| 482 | CASSLDNQDTQYF | 28 |
| 483 | CASSLDNSGNTLYF | 28 |
| 484 | CASSLDNYAEQFF | 28 |
| 485 | CASSLDRYAEQFF | 28 |
| 486 | CASSLDSAETLYF | 28 |
| 487 | CASSLDSDYTF | 28 |
| 488 | CASSLDSQNTLYF | 28 |
| 489 | CASSLDSSAETLYF | 28 |
| 490 | CASSLDSSGNTLYF | 28 |
| 491 | CASSLDSSQNTLYF | 28 |
| 492 | CASSLDSYAEQFF | 28 |
| 493 | CASSLDTEVFF | 28 |
| 494 | CASSLDTGQLYF | 28 |
| 495 | CASSLDWGNYAEQFF | 28 |
| 496 | CASSLDWGQDTQYF | 28 |
| 497 | CASSLDWGSAETLYF | 28 |
| 498 | CASSLEANSDYTF | 28 |
| 499 | CASSLEDSGNTLYF | 28 |
| 500 | CASSLEGAETLYF | 28 |
| 501 | CASSLEGNSDYTF | 28 |
| 502 | CASSLEGNTEVFF | 28 |
| 503 | CASSLEGNTGQLYF | 28 |
| 504 | CASSLEGNTLYF | 28 |
| 505 | CASSLEGNYAEQFF | 28 |
| 506 | CASSLEGSGNTLYF | 28 |
| 507 | CASSLEGSQNTLYF | 28 |
| 508 | CASSLEGYAEQFF | 28 |
| 509 | CASSLESANSIDYTF | 28 |
| 510 | CASSLGAETLYF | 28 |
| 511 | CASSLGANSDYTF | 28 |
| 512 | CASSLGDQDTQYF | 28 |
| 513 | CASSLGENTLYF | 28 |
| 514 | CASSLGERLFF | 28 |
| 515 | CASSLGETLYF | 28 |
| 516 | CASSLGGAETLYF | 28 |
| 517 | CASSLGGNSDYTF | 28 |
| 518 | CASSLGGNTEVFF | 28 |
| 519 | CASSLGGNTLYF | 28 |
| 520 | CASSLGGSAETLYF | 28 |
| 521 | CASSLGGSQNTLYF | 28 |
| 522 | CASSLGNQDTQYF | 28 |
| 523 | CASSLGNSGNTLYF | 28 |
| 524 | CASSLGNTEVFF | 28 |
| 525 | CASSLGNTGQLYF | 28 |
| 526 | CASSLGNTLYF | 28 |
| 527 | CASSLGQNSDYTF | 28 |
| 528 | CASSLGQNTLYF | 28 |
| 529 | CASSLGQNYAEQFF | 28 |
| 530 | CASSLGQQNTLYF | 28 |
| 531 | CASSLGQSQNTLYF | 28 |
| 532 | CASSLGQTEVFF | 28 |
| 533 | CASSLGSAETLYF | 28 |
| 534 | CASSLGSQNTLYF | 28 |
| 535 | CASSLGSSAETLYF | 28 |
| 536 | CASSLGTANSDYTF | 28 |
| 537 | CASSLGTTNSDYTF | 28 |
| 538 | CASSLLGNYAEQFF | 28 |
| 539 | CASSLNQDTQYF | 28 |
| 540 | CASSLNSAETLYF | 28 |
| 541 | CASSLNTGQLYF | 28 |
| 542 | CASSLQANSDYTF | 28 |
| 543 | CASSLQANTEVFF | 28 |
| 544 | CASSLQGAETLYF | 28 |
| 545 | CASSLQGAGNTLYF | 28 |
| 546 | CASSLQGANTEVFF | 28 |
| 547 | CASSLQGNSDYTF | 28 |
| 548 | CASSLQGNTEVFF | 28 |
| 549 | CASSLQGNTGQLYF | 28 |
| 550 | CASSLQGSAETLYF | 28 |
| 551 | CASSLQGSGNTLYF | 28 |
| 552 | CASSLQGSQNTLYF | 28 |
| 553 | CASSLQGTEVFF | 28 |
| 554 | CASSLQNTLYF | 28 |
| 555 | CASSLRGSQNTLYF | 28 |
| 556 | CASSLSAETLYF | 28 |
| 557 | CASSLSGNTLYF | 28 |
| 558 | CASSLSGSNYAEQFF | 28 |
| 559 | CASSLSQNTLYF | 28 |
| 560 | CASSLSQQNTLYF | 28 |
| 561 | CASSLSSQNTLYF | 28 |
| 562 | CASSLTANSDYTF | 28 |
| 563 | CASSLTANTEVFF | 28 |
| 564 | CASSLTDYNSPLYF | 28 |
| 565 | CASSLTGGYAEQFF | 28 |
| 566 | CASSLTGNYAEQFF | 28 |
| 567 | CASSLTGSQNTLYF | 28 |
| 568 | CASSLTSAETLYF | 28 |
| 569 | CASSLWGSQNTLYF | 28 |
| 570 | CASSPDSAETLYF | 28 |
| 571 | CASSPDSSGNTLYF | 28 |
| 572 | CASSPDSSQNTLYF | 28 |
| 573 | CASSPDWGENTLYF | 28 |
| 574 | CASSPDWGNYAEQFF | 28 |
| 575 | CASSPGDQDTQYF | 28 |
| 576 | CASSPGGQNTLYF | 28 |
| 577 | CASSPGHERLFF | 28 |
| 578 | CASSPGQGNSDYTF | 28 |
| 579 | CASSPGQGYAEQFF | 28 |
| 580 | CASSPGQNTEVFF | 28 |
| 581 | CASSPGQNYAEQFF | 28 |
| 582 | CASSPGQSQNTLYF | 28 |
| 583 | CASSPGSQNTLYF | 28 |
| 584 | CASSPGTANTEVFF | 28 |
| 585 | CASSPNTGQLYF | 28 |
| 586 | CASSPNYAEQFF | 28 |
| 587 | CASSPQGNTEVFF | 28 |
| 588 | CASSPSAETLYF | 28 |
| 589 | CASSPSQNTLYF | 28 |
| 590 | CASSPSSAETLYF | 28 |
| 591 | CASSPTANSDYTF | 28 |
| 592 | CASSPTASQNTLYF | 28 |
| 593 | CASSPTGAETLYF | 28 |
| 594 | CASSPTGSQNTLYF | 28 |
| 595 | CASSPYAEQFF | 28 |
| 596 | CASSQDTEVFF | 28 |
| 597 | CASSQDWGQDTQYF | 28 |
| 598 | CASSQEASNSDYTF | 28 |
| 599 | CASSQGLGDTLYF | 28 |
| 600 | CASSQGQNTEVFF | 28 |
| 601 | CASSQGSQNTLYF | 28 |
| 602 | CASSQNTGQLYF | 28 |
| 603 | CASSQQGSQNTLYF | 28 |
| 604 | CASSQSLDNQDTQYF | 28 |
| 605 | CASSRDIQDTQYF | 28 |
| 606 | CASSRDIYAEQFF | 28 |
| 607 | CASSRDNNERLFF | 28 |
| 608 | CASSRDNQDTQYF | 28 |
| 609 | CASSRDNYAEQFF | 28 |
| 610 | CASSRDRDTEVFF | 28 |
| 611 | CASSRDSAFTLYF | 28 |
| 612 | CASSRDSQNTLYF | 28 |
| 613 | CASSRDSSAETLYF | 28 |
| 614 | CASSRDSYAEQFF | 28 |
| 615 | CASSRDWEQNTLYF | 28 |
| 616 | CASSRDWGNYAEQFF | 28 |
| 617 | CASSRDWGQDTQYF | 28 |
| 618 | CASSRGQNTLYF | 28 |
| 619 | CASSRGSAETLYF | 28 |
| 620 | CASSRQANTEVFF | 28 |
| 621 | CASSRQGANSDYTF | 28 |
| 622 | CASSRQGNSDYTF | 28 |
| 623 | CASSRQGNTEVFF | 28 |
| 624 | CASSRQGNTGQLYF | 28 |
| 625 | CASSRQGNYAEQFF | 28 |
| 626 | CASSRQGSQNTLYF | 28 |
| 627 | CASSRQNSDYTF | 28 |
| 628 | CASSRQNTEVFF | 28 |
| 629 | CASSRSQNTLYF | 28 |
| 630 | CASSRVGSDYTF | 28 |
| 631 | CASSSAETLYF | 28 |
| 632 | CASSSDSAETLYF | 28 |
| 633 | CASSSDWGNYAEQFF | 28 |
| 634 | CASSSGDQDTQYF | 28 |
| 635 | CASSSGQNTEVFF | 28 |
| 636 | CASSSGSQNTLYF | 28 |
| 637 | CASSSGTANTEVFF | 28 |
| 638 | CASSSNTGQLYF | 28 |
| 639 | CASSSQGAETLYF | 28 |
| 640 | CASSSQNTLYF | 28 |
| 641 | CASSSSAETLYF | 28 |
| 642 | CASSSSQNTLYF | 28 |
| 643 | CASSTSAETLYF | 28 |
| 644 | CASSWDNYAEQFF | 28 |
| 645 | CASSWDSAETLYF | 28 |
| 646 | CASSWGQNTLYF | 28 |
| 647 | CASSWGSAETLYF | 28 |
| 648 | CASSYSAETLYF | 28 |
| 649 | CASSYSQNTLYF | 28 |
| 650 | CAWSLQGYNSPLYF | 28 |
| 651 | CASGDAGAETLYF | 27 |
| 652 | CASGDAGDTQYF | 27 |
| 653 | CASGDAGGQNTLYF | 27 |
| 654 | CASGDAGGYAEQFF | 27 |
| 655 | CASGDAGNTGQLYF | 27 |
| 656 | CASGDAGQNTEVFF | 27 |
| 657 | CASGDAGSAETLYF | 27 |
| 658 | CASGDANSDYTF | 27 |
| 659 | CASGDARDTQYF | 27 |
| 660 | CASGDASAETLYF | 27 |
| 661 | CASGDNTEVFF | 27 |
| 662 | CASGDNYNSPLYF | 27 |
| 663 | CASGDSSAETLYF | 27 |
| 664 | CASGDWGNQDTQYF | 27 |
| 665 | CASGDWGNYAEQFF | 27 |
| 666 | CASGENTLYF | 27 |
| 667 | CASGGQNYAEQFF | 27 |
| 668 | CASGGSQNTLYF | 27 |
| 669 | CASGQNTLYF | 27 |
| 670 | CASKTANQDTQYF | 27 |
| 671 | CASRDNSGNTLYF | 27 |
| 672 | CASRDSSQNTLYF | 27 |
| 673 | CASRRDSAETLYF | 27 |
| 674 | CASRTGGYAEQFF | 27 |
| 675 | CASRTSAETLYF | 27 |
| 676 | CASSASAETLYF | 27 |
| 677 | CASSDAGQNTLYF | 27 |
| 678 | CASSDANSDYTF | 27 |
| 679 | CASSDGSQNTLYF | 27 |
| 680 | CASSDNQDTQYF | 27 |
| 681 | CASSDNYAEQFF | 27 |
| 682 | CASSDSSGNTLYF | 27 |
| 683 | CASSDWGSAETLYF | 27 |
| 684 | CASSESQNTLYF | 27 |
| 685 | CASSFDSAETLYF | 27 |
| 686 | CASSFDTEVFF | 27 |
| 687 | CASSFGGQNTLYF | 27 |
| 688 | CASSFQNTLYF | 27 |
| 689 | CASSFSSAETLYF | 27 |
| 690 | CASSGAETLYF | 27 |
| 691 | CASSGQANTEVFF | 27 |
| 692 | CASSGQNYAEQFF | 27 |
| 693 | CASSGTASAETLYF | 27 |
| 694 | CASSGTGGYAEQFF | 27 |
| 695 | CASSGTISNERLFF | 27 |
| 696 | CASSGTVSNERLFF | 27 |
| 697 | CASSLAETLYF | 27 |
| 698 | CASSLAGGYAEQFF | 27 |
| 699 | CASSLAGSGNTLYF | 27 |
| 700 | CASSLANSGNTLYF | 27 |
| 701 | CASSLASQNTLYF | 27 |
| 702 | CASSLDAETLYF | 27 |
| 703 | CASSLDANSDYTF | 27 |
| 704 | CASSLDNSDYTF | 27 |
| 705 | CASSLDNSQNTLYF | 27 |
| 706 | CASSLDNTEVFF | 27 |
| 707 | CASSLDQNTLYF | 27 |
| 708 | CASSLDRDTEVFF | 27 |
| 709 | CASSLDREVFF | 27 |
| 710 | CASSLDRGAETLYF | 27 |
| 711 | CASSLDRNSDYTF | 27 |
| 712 | CASSLDRNTEVFF | 27 |
| 713 | CASSLDRNTGQLYF | 27 |
| 714 | CASSLDINSDYTF | 27 |
| 715 | CASSLDWGNTGQLYF | 27 |
| 716 | CASSLDWGQNTLYF | 27 |
| 717 | CASSLDWGSQNTLYF | 27 |
| 718 | CASSLDWGYAEQFF | 27 |
| 719 | CASSLDYAEQFF | 27 |
| 720 | CASSLEANTEVFF | 27 |
| 721 | CASSLEGAGNTLYF | 27 |
| 722 | CASSLEGGQNTLYF | 27 |
| 723 | CASSLEGSDYTF | 27 |
| 724 | CASSLEGTGQLYF | 27 |
| 725 | CASSLENTEVFF | 27 |
| 726 | CASSLENTLYF | 27 |
| 727 | CASSLEQNSDYTF | 27 |
| 728 | CASSLETLYF | 27 |
| 729 | CASSLGANTEVFF | 27 |
| 730 | CASSLGANTGQLYF | 27 |
| 731 | CASSLGDTEVFF | 27 |
| 732 | CASSLGGNTGQLYF | 27 |
| 733 | CASSLGGNYAEQFF | 27 |
| 734 | CASSLGGSGNTLYF | 27 |
| 735 | CASSLGGTEVFF | 27 |
| 736 | CASSLGGYAEQFF | 27 |
| 737 | CASSLGNYAEQFF | 27 |
| 738 | CASSLGQANTEVFF | 27 |
| 739 | CASSLGQGAETLYF | 27 |
| 740 | CASSLGQGNTEVFF | 27 |
| 741 | CASSLGQGYAEQFF | 27 |
| 742 | CASSLGQYAEQEF | 27 |
| 743 | CASSLGTANTEVFF | 27 |
| 744 | CASSLGTEVFF | 27 |
| 745 | CASSLGTNTEVFF | 27 |
| 746 | CASSLGVNQDTQYF | 27 |
| 747 | CASSLGVYAEQFF | 27 |
| 748 | CASSLLGGYAEQFF | 27 |
| 749 | CASSLNSDYTF | 27 |
| 750 | CASSLNSQNTLYF | 27 |
| 751 | CASSLNTEVFF | 27 |
| 752 | CASSLQGANSDYTF | 27 |
| 753 | CASSLQGNTLYF | 27 |
| 754 | CASSLQGNYAEQFF | 27 |
| 755 | CASSLQGQNTLYF | 27 |
| 756 | CASSLQGYAEQFF | 27 |
| 757 | CASSLQNSDYTF | 27 |
| 758 | CASSLQNTEVFF | 27 |
| 759 | CASSLRGQNTLYF | 27 |
| 760 | CASSLRSQNTLYF | 27 |
| 761 | CASSLSGGQNTLYF | 27 |
| 762 | CASSLSSAETLYF | 27 |
| 763 | CASSLTANTGQLYF | 27 |
| 764 | CASSLTGAETLYF | 27 |
| 765 | CASSLIGANTGQLYF | 27 |
| 766 | CASSLTGGQNTLYF | 27 |
| 767 | CASSLTTNTEVFF | 27 |
| 768 | CASSLTTSAETLYF | 27 |
| 769 | CASSLVGNQDTQYF | 27 |
| 770 | CASSLVGSQNTLYF | 27 |
| 771 | CASSNSAETLYF | 27 |
| 772 | CASSPAETLYF | 27 |
| 773 | CASSPIDSNERLFF | 27 |
| 774 | CASSPDWGSQNTLYF | 27 |
| 775 | CASSPGAGSNERLFF | 27 |
| 776 | CASSPGLGNYAEQFF | 27 |
| 777 | CASSPGLGQDTQYF | 27 |
| 778 | CASSPGNTEVFF | 27 |
| 779 | CASSPGQGAETLYF | 27 |
| 780 | CASSPGQNTGQLYF | 27 |
| 781 | CASSPGSAETLYF | 27 |
| 782 | CASSPGTANTGQLYF | 27 |
| 783 | CASSPGTTNSDYTF | 27 |
| 784 | CASSPQGAETLYF | 27 |
| 785 | CASSPQGSAETLYF | 27 |
| 786 | CASSPTANTEVFF | 27 |
| 787 | CASSPTGGQNTLYF | 27 |
| 788 | CASSQDSAETLYF | 27 |
| 789 | CASSQDSSGNTLYF | 27 |
| 790 | CASSQDSSQNTLYF | 27 |
| 791 | CASSQDWGNYAEQFF | 27 |
| 792 | CASSQGAGNTLYF | 27 |
| 793 | CASSQNTLYF | 27 |
| 794 | CASSQQGNTEVFF | 27 |
| 795 | CASSQTSAETLYF | 27 |
| 796 | CASSRAETLYF | 27 |
| 797 | CASSRDKNTLYF | 27 |
| 798 | CASSRDNQNTLYF | 27 |
| 799 | CASSRDNSDYTF | 27 |
| 800 | CASSRDNSQNTLYF | 27 |
| 801 | CASSRDRDAEQFF | 27 |
| 802 | CASSRDREVFF | 27 |
| 803 | CASSRDRGNTLYF | 27 |
| 804 | CASSRDRNTEVFF | 27 |
| 805 | CASSRDSNERLFF | 27 |
| 806 | CASSRDSNYAEQFF | 27 |
| 807 | CASSRDSSQNTLYF | 27 |
| 808 | CASSRDWGNTLYF | 27 |
| 809 | CASSRDWGSQNTLYF | 27 |
| 810 | CASSRDWGYAEQFF | 27 |
| 811 | CASSRGSQNTLYF | 27 |
| 812 | CASSRLGNYAEQFF | 27 |
| 813 | CASSRQGAETLYF | 27 |
| 814 | CASSRQGNTLYF | 27 |
| 815 | CASSRQGQNTLYF | 27 |
| 816 | CASSRQNYAEQFF | 27 |
| 817 | CASSRTGENTLYF | 27 |
| 818 | CASSRTGQNTLYF | 27 |
| 819 | CASSSGGQNTLYF | 27 |
| 820 | CASSSGNTLYF | 27 |
| 821 | CASSSGNYAEQFF | 27 |
| 822 | CASSSGQQNTLYF | 27 |
| 823 | CASSSGTTNTEVFF | 27 |
| 824 | CASSSQGANTEVFF | 27 |
| 825 | CASSSTANSDYTF | 27 |
| 826 | CASSWGSQNTLYF | 27 |
| 827 | CASSYGQNTLYF | 27 |
| 828 | CGARDSAETLYF | 27 |
| 829 | CSAGGQNTLYF | 27 |
| 830 | CASGAETLYF | 26 |
| 831 | CASGDADSGNTLYF | 26 |
| 832 | CASGDAETLYF | 26 |
| 833 | CASGDAGDQDTQYF | 26 |
| 834 | CASGDAGEQYF | 26 |
| 835 | CASGDAGGNTLYF | 26 |
| 836 | CASGDAGNSDYTF | 26 |
| 837 | CASGDANSGNTLYF | 26 |
| 838 | CASGDASQNTLYF | 26 |
| 839 | CASGDAYAEQFF | 26 |
| 840 | CASGDNSGNTLYF | 26 |
| 841 | CASGDPGNYAEQFF | 26 |
| 842 | CASGDRGNTEVFF | 26 |
| 843 | CASGDRYEQYF | 26 |
| 844 | CASGDSQNTLYF | 26 |
| 845 | CASGDSSGNTLYF | 26 |
| 846 | CASGDSYAEQFF | 26 |
| 847 | CASGDYAEQFF | 26 |
| 848 | CASGEGQNTLYF | 26 |
| 849 | CASGESAETLYF | 26 |
| 850 | CASGGQANTEVFF | 26 |
| 851 | CASRDWGNYAEQFF | 26 |
| 852 | CASRGAETLYF | 26 |
| 853 | CASSASQNTLYF | 26 |
| 854 | CASSDAGNTLYF | 26 |
| 855 | CASSDAGSQNTLYF | 26 |
| 856 | CASSDASAETLYF | 26 |
| 857 | CASSDRAETLYF | 26 |
| 858 | CASSDRDTEVFF | 26 |
| 859 | CASSDRNTEVFF | 26 |
| 860 | CASSDWDQDTQYF | 26 |
| 861 | CASSDWGNYAEQFF | 26 |
| 862 | CASSDWGQNTLYF | 26 |
| 863 | CASSFGAETLYF | 26 |
| 864 | CASSFGGAETLYF | 26 |
| 865 | CASSFGSAETLYF | 26 |
| 866 | CASSFPSGNTLYF | 26 |
| 867 | CASSFQGNTEVFF | 26 |
| 868 | CASSFSGAQDTQYF | 26 |
| 869 | CASSGDSQNTLYF | 26 |
| 870 | CASSGDSYAEQFF | 26 |
| 871 | CASSGLGNYAEQFF | 26 |
| 872 | CASSGQGAETLYF | 26 |
| 873 | CASSGTANTGQLYF | 26 |
| 874 | CASSGTSNSDYTF | 26 |
| 875 | CASSLAANTGQLYF | 26 |
| 876 | CASSLAGDTGQLYF | 26 |
| 877 | CASSLAGNQDTQYF | 26 |
| 878 | CASSLAGNTEVFF | 26 |
| 879 | CASSLAGNTGQLYF | 26 |
| 880 | CASSLAGSAETLYF | 26 |
| 881 | CASSLDANTEVFF | 26 |
| 882 | CASSLDGNTLYF | 26 |
| 883 | CASSLDIYAEQFF | 26 |
| 884 | CASSLDNERLFF | 26 |
| 885 | CASSLDNNQDTQYF | 26 |
| 886 | CASSLDQAPLF | 26 |
| 887 | CASSLDRNTLYF | 26 |
| 888 | CASSLDSGNTLYF | 26 |
| 889 | CASSLDSNQDTQYF | 26 |
| 890 | CASSLDSQDTQYF | 26 |
| 891 | CASSLDTSQNTLYF | 26 |
| 892 | CASSLDWGNTLYF | 26 |
| 893 | CASSLEANTGQLYF | 26 |
| 894 | CASSLEDTGQLYF | 26 |
| 895 | CASSLEGANSDYTF | 26 |
| 896 | CASSLEGQNTLYF | 26 |
| 897 | CASSLEGSAETLYF | 26 |
| 898 | CASSLENTGQLYF | 26 |
| 899 | CASSLESAETLYF | 26 |
| 900 | CASSLGASQNTLYF | 26 |
| 901 | CASSLGDNYAEQFF | 26 |
| 902 | CASSLGDSAETLYF | 26 |
| 903 | CASSLGDSGNTLYF | 26 |
| 904 | CASSLGDTGQLYF | 26 |
| 905 | CASSLGGSNERLFF | 26 |
| 906 | CASSLGQNTGQLYF | 26 |
| 907 | CASSLGQSAETLYF | 26 |
| 908 | CASSLGSGNTLYF | 26 |
| 909 | CASSLGTGQLYF | 26 |
| 910 | CASSLGTSAETLYF | 26 |
| 911 | CASSLGTTNTEVFF | 26 |
| 912 | CASSLGVSNERLFF | 26 |
| 913 | CASSLLGDQDTQYF | 26 |
| 914 | CASSLQGGNTLYF | 26 |
| 915 | CASSLQGNQDTQYF | 26 |
| 916 | CASSLQISNERLFF | 26 |
| 917 | CASSLQSQNTLYF | 26 |
| 918 | CASSLRAETLYF | 26 |
| 919 | CASSLRDNQDTQYF | 26 |
| 920 | CASSLRDTGQLYF | 26 |
| 921 | CASSLRGNTEVFF | 26 |
| 922 | CASSLRGSGNTLYF | 26 |
| 923 | CASSLSGSAETLYF | 26 |
| 924 | CASSLSNERLFF | 26 |
| 925 | CASSLSTGQLYF | 26 |
| 926 | CASSLTASQNTLYF | 26 |
| 927 | CASSLTDSGNTLYF | 26 |
| 928 | CASSLTENTLYF | 26 |
| 929 | CASSLTGANTEVFF | 26 |
| 930 | CASSLTGDQDTQYF | 26 |
| 931 | CASSLTGGNTLYF | 26 |
| 932 | CASSLTGGSQNTLYF | 26 |
| 933 | CASSLTGNTLYF | 26 |
| 934 | CASSLTGQNTLYF | 26 |
| 935 | CASSLTGSAETLYF | 26 |
| 936 | CASSLTNTEVFF | 26 |
| 937 | CASSLTSSAETLYF | 26 |
| 938 | CASSLVGNYAEQFF | 26 |
| 939 | CASSLVNQDTQYF | 26 |
| 940 | CASSLVSQNTLYF | 26 |
| 941 | CASSLWGNYAEQFF | 26 |
| 942 | CASSPDNSGNTLYF | 26 |
| 943 | CASSPDSQNTLYF | 26 |
| 944 | CASSPDWGQNTLYF | 26 |
| 945 | CASSPGAETLYF | 26 |
| 946 | CASSPGGNYAEQFF | 26 |
| 947 | CASSPGQYAEQFF | 26 |
| 948 | CASSPGQYNSPLYF | 26 |
| 949 | CASSPGTSSAETLYF | 26 |
| 950 | CASSPGTTNTEVFF | 26 |
| 951 | CASSPGTTSAETLYF | 26 |
| 952 | CASSPQANTGQLYF | 26 |
| 953 | CASSPQGAGNTLYF | 26 |
| 954 | CASSPQGNTGQLYF | 26 |
| 955 | CASSPQGSQNTLYF | 26 |
| 956 | CASSPTANTGQLYF | 26 |
| 957 | CASSPTGGNYAEQFF | 26 |
| 958 | CASSPTGNTGQLYF | 26 |
| 959 | CASSPTGNYAEQFF | 26 |
| 960 | CASSPTSAETLYF | 26 |
| 961 | CASSPTSSQNTLYF | 26 |
| 962 | CASSQDSQNTLYF | 26 |
| 963 | CASSQDWGSQNTLYF | 26 |
| 964 | CASSQGNYAEQFF | 26 |
| 965 | CASSQGSAETLYF | 26 |
| 966 | CASSQGSGNTLYF | 26 |
| 967 | CASSQGTANTGQLYF | 26 |
| 968 | CASSQNTEVFF | 26 |
| 969 | CASSQQGANTEVFF | 26 |
| 970 | CASSRDNSAETLYF | 26 |
| 971 | CASSRDNSGNTLYF | 26 |
| 972 | CASSRDRGAEQFF | 26 |
| 973 | CASSRDRNTGQLYF | 26 |
| 974 | CASSRDSSGNTLYF | 26 |
| 975 | CASSRDTGQLYF | 26 |
| 976 | CASSRDWGNTGQLYF | 26 |
| 977 | CASSRLGQDTQYF | 26 |
| 978 | CASSRNTGQLYF | 26 |
| 979 | CASSRQANSDYTF | 26 |
| 980 | CASSRQGANTEVFF | 26 |
| 981 | CASSRQGANTGQLYF | 26 |
| 982 | CASSRQNTGQLYF | 26 |
| 983 | CASSRTDSGNTLYF | 26 |
| 984 | CASSRTSQNTLYF | 26 |
| 985 | CASSSDSQNTLYF | 26 |
| 986 | CASSSDWGQDTQYF | 26 |
| 987 | CASSSGNTEVFF | 26 |
| 988 | CASSSGQANTEVFF | 26 |
| 989 | CASSSGQNTLYF | 26 |
| 990 | CASSSGQQDTQYF | 26 |
| 991 | CASSSGTNTEVFF | 26 |
| 992 | CASSSQGNSDYTF | 26 |
| 993 | CASSSQGNTEVFT | 26 |
| 994 | CASSSQGNTGQLYF | 26 |
| 995 | CASSSSGNTLYF | 26 |
| 996 | CASSSTANTEVFF | 26 |
| 997 | CASSSTASQNTLYF | 26 |
| 998 | CASSSTGNTEVFF | 26 |
| 999 | CASSSTSAETLYF | 26 |
| 1000 | CASSSYAEQFF | 26 |
| 1001 | CASSTGNTGQLYF | 26 |
| 1002 | CASSWDSQNTLYF | 26 |
| 1003 | CASSWDSYAEQFF | 26 |
| 1004 | CASSWGNYAEQFF | 26 |
| 1005 | CSKDSAETLYF | 26 |
| 1006 | CSSSQGTNERLFF | 26 |
| 1007 | CASGDADEQYF | 25 |
| 1008 | CASGDADTGQLYF | 25 |
| 1009 | CASGDAGAEQFF | 25 |
| 1010 | CASGDAGANSDYTF | 25 |
| 1011 | CASGDAGDTGQLYF | 25 |
| 1012 | CASGDAGGAETLYF | 25 |
| 1013 | CASGDAGNQDTQYF | 25 |
| 1014 | CASGDAGNTEVFF | 25 |
| 1015 | CASGDAGNYAEQFF | 25 |
| 1016 | CASGDAGQDTQYF | 25 |
| 1017 | CASGDAQSGNTLYF | 25 |
| 1018 | CASGDDQDTQYF | 25 |
| 1019 | CASGDGGQNTLYF | 25 |
| 1020 | CASGDNSQNTLYF | 25 |
| 1021 | CASGDPSAETLYF | 25 |
| 1022 | CASGDPSQNTLYF | 25 |
| 1023 | CASGDRGSGNTLYF | 25 |
| 1024 | CASGDRGSQNTLYF | 25 |
| 1025 | CASGDRNTEVFF | 25 |
| 1026 | CASGDSNERLFF | 25 |
| 1027 | CASGEGGQNTLYF | 25 |
| 1028 | CASGGQGNTEVFF | 25 |
| 1029 | CASGGTANTEVFF | 25 |
| 1030 | CASGSAETLYF | 25 |
| 1031 | CASRDNYAEQFF | 25 |
| 1032 | CASRDSNYAEQFF | 25 |
| 1033 | CASRDSSGNTLYF | 25 |
| 1034 | CASRDWGSAETLYF | 25 |
| 1035 | CASRGQNYAEQFF | 25 |
| 1036 | CASSDAGGQNTLYF | 25 |
| 1037 | CASSDANTEVFF | 25 |
| 1038 | CASSDGAETLYF | 25 |
| 1039 | CASSDGGNYAEQFF | 25 |
| 1040 | CASSDNSGNTLYF | 25 |
| 1041 | CASSDNSQNTLYF | 25 |
| 1042 | CASSDNTEVFF | 25 |
| 1043 | CASSDRGAETLYF | 25 |
| 1044 | CASSDRNSDYTF | 25 |
| 1045 | CASSEGQNTLYF | 25 |
| 1046 | CASSEGSQNTLYF | 25 |
| 1047 | CASSENSGNTLYF | 25 |
| 1048 | CASSENTGQLYF | 25 |
| 1049 | CASSETANTEVFF | 25 |
| 1050 | CASSFGGNYAEQFF | 25 |
| 1051 | CASSFGGSAETLYF | 25 |
| 1052 | CASSFGTANTEVFF | 25 |
| 1053 | CASSFNYAEQFF | 25 |
| 1054 | CASSGQGNTEVFF | 25 |
| 1055 | CASSGQGQNTLYF | 25 |
| 1056 | CASSGSAETLYF | 25 |
| 1057 | CASSGSQNTLYF | 25 |
| 1058 | CASSGTGNSDYTF | 25 |
| 1059 | CASSGTGQNTLYF | 25 |
| 1060 | CASSGTTNTGQLYF | 25 |
| 1061 | CASSINQDTQYF | 25 |
| 1062 | CASSLAANTEVFF | 25 |
| 1063 | CASSLAGAETLYF | 25 |
| 1064 | CASSLAGDQDTQYF | 25 |
| 1065 | CASSLAGENTLYF | 25 |
| 1066 | CASSLAGQNTLYF | 25 |
| 1067 | CASSLDGGQNTLYF | 25 |
| 1068 | CASSLDGNTEVFF | 25 |
| 1069 | CASSLDGYAEQFF | 25 |
| 1070 | CASSLDNTGQLYF | 25 |
| 1071 | CASSLDRAGNTLYF | 25 |
| 1072 | CASSLDRANSDYTF | 25 |
| 1073 | CASSLDRDAEQFF | 25 |
| 1074 | CASSLDRGEVFF | 25 |
| 1075 | CASSLDRTEVFF | 25 |
| 1076 | CASSLEGANTEVFF | 25 |
| 1077 | CASSLEGDQDTQYF | 25 |
| 1078 | CASSLEGDSDYTF | 25 |
| 1079 | CASSLEGDTGQLYF | 25 |
| 1080 | CASSLEGSSAETLYF | 25 |
| 1081 | CASSLEGTSAETLYF | 25 |
| 1082 | CASSLENSDYTF | 25 |
| 1083 | CASSLESQNTLYF | 25 |
| 1084 | CASSLGGGQNTLYF | 25 |
| 1085 | CASSLGGGYAEQFF | 25 |
| 1086 | CASSLGGSDYTF | 25 |
| 1087 | CASSLGHQDTQYF | 25 |
| 1088 | CASSLGISNERLFF | 25 |
| 1089 | CASSLGLGAETLYF | 25 |
| 1090 | CASSLGLGYAEQFF | 25 |
| 1091 | CASSLGNSAETLYF | 25 |
| 1092 | CASSLGQGTEVFF | 25 |
| 1093 | CASSLGSSQNTLYF | 25 |
| 1094 | CASSLGTANTGQLYF | 25 |
| 1095 | CASSLGTASAETLYF | 25 |
| 1096 | CASSLGISSAETLYF | 25 |
| 1097 | CASSLGVNYAEQFF | 25 |
| 1098 | CASSLGVSQNTLYF | 25 |
| 1099 | CASSLLGANTGQLYF | 25 |
| 1100 | CASSLPSAETLYF | 25 |
| 1101 | CASSLQGANTGQLYF | 25 |
| 1102 | CASSLQGDQDTQYF | 25 |
| 1103 | CASSLQGSDYTF | 25 |
| 1104 | CASSLQQDTQYF | 25 |
| 1105 | CASSLRQNTEVFF | 25 |
| 1106 | CASSLRSAETLYF | 25 |
| 1107 | CASSLSGAETLYF | 25 |
| 1108 | CASSLSGNYAEQEF | 25 |
| 1109 | CASSLSGQNTLYF | 25 |
| 1110 | CASSLSGSGNTLYF | 25 |
| 1111 | CASSLSGSQNTLYF | 25 |
| 1112 | CASSLSNTEVFF | 25 |
| 1113 | CASSLTASAETLYF | 25 |
| 1114 | CASSLTGDYAEQFF | 25 |
| 1115 | CASSLTGENTLYF | 25 |
| 1116 | CASSLTGGAETLYF | 25 |
| 1117 | CASSLTGNTGQLYF | 25 |
| 1118 | CASSLTSQNTLYF | 25 |
| 1119 | CASSLVSAETLYF | 25 |
| 1120 | CASSNTGQLYF | 25 |
| 1121 | CASSNYAEQFF | 25 |
| 1122 | CASSPDNYAEQFF | 25 |
| 1123 | CASSPDRNTEVFF | 25 |
| 1124 | CASSPDRSQNTLYF | 25 |
| 1125 | CASSPDWGQDTQYF | 25 |
| 1126 | CASSPGGAETLYF | 25 |
| 1127 | CASSPGGSQNTLYF | 25 |
| 1128 | CASSPGNSDYTF | 25 |
| 1129 | CASSPGQANTEVFF | 25 |
| 1130 | CASSPGQGQNTLYF | 25 |
| 1131 | CASSPGQGTEVFF | 25 |
| 1132 | CASSPGQNQDTQYF | 25 |
| 1133 | CASSPGQNSDYTF | 25 |
| 1134 | CASSPGQNTLYF | 25 |
| 1135 | CASSPGQQNTLYF | 25 |
| 1136 | CASSPGQTEVFF | 25 |
| 1137 | CASSPGTAETLYF | 25 |
| 1138 | CASSPGTANSDYTF | 25 |
| 1139 | CASSPGTASAETLYF | 25 |
| 1140 | CASSPGTGYAEQFF | 25 |
| 1141 | CASSPGTNTEVFF | 25 |
| 1142 | CASSPGTTNERLFF | 25 |
| 1143 | CASSPLGSQNTLYF | 25 |
| 1144 | CASSPTGGYAEQFF | 25 |
| 1145 | CASSPTGNQDTQYF | 25 |
| 1146 | CASSPTTSQNTLYF | 25 |
| 1147 | CASSPTVSNERLFF | 25 |
| 1148 | CASSPTVSQNTLYF | 25 |
| 1149 | CASSQANTGQLYF | 25 |
| 1150 | CASSQDSYAEQFF | 25 |
| 1151 | CASSQEGSQNTLYF | 25 |
| 1152 | CASSQGAETLYF | 25 |
| 1153 | CASSQGGQNTLYF | 25 |
| 1154 | CASSQGNTGQLYF | 25 |
| 1155 | CASSQGQNTLYF | 25 |
| 1156 | CASSQGQNYAEQFF | 25 |
| 1157 | CASSQNSGNTLYF | 25 |
| 1158 | CASSQQGAETLYF | 25 |
| 1159 | CASSQQGNTGQLYF | 25 |
| 1160 | CASSQQGSGNTLYF | 25 |
| 1161 | CASSRDISQNTLYF | 25 |
| 1162 | CASSRDNNYAEQFF | 25 |
| 1163 | CASSRDNQAPLF | 25 |
| 1164 | CASSRDNTEVFF | 25 |
| 1165 | CASSRDRANSDYTF | 25 |
| 1166 | CASSRDRNTLYF | 25 |
| 1167 | CASSRDRNYAEQFF | 25 |
| 1168 | CASSRDSANSDYTF | 25 |
| 1169 | CASSRDTEVFF | 25 |
| 1170 | CASSRGAETLYF | 25 |
| 1171 | CASSRGNQDTQYF | 25 |
| 1172 | CASSRGNYAEQFF | 25 |
| 1173 | CASSRLGSQNTLYF | 25 |
| 1174 | CASSRNQDTQYF | 25 |
| 1175 | CASSRNTEVFF | 25 |
| 1176 | CASSRQGAGNTLYF | 25 |
| 1177 | CASSRQGDTEVFF | 25 |
| 1178 | CASSRQSQNTLYF | 25 |
| 1179 | CASSRTANSDYTF | 25 |
| 1180 | CASSRTASAETLYF | 25 |
| 1181 | CASSRTGGQNTLYF | 25 |
| 1182 | CASSRTGGYAEQFF | 25 |
| 1183 | CASSRTGNSDYTF | 25 |
| 1184 | CASSRTGNTGQLYF | 25 |
| 1185 | CASSRTGSQNTLYF | 25 |
| 1186 | CASSRTNTEVFF | 25 |
| 1187 | CASSRTTNTEVFF | 25 |
| 1188 | CASSRYAEQFF | 25 |
| 1189 | CASSSAANTEVFF | 25 |
| 1190 | CASSSANTGQLYF | 25 |
| 1191 | CASSSETLYF | 25 |
| 1192 | CASSSGAFTLYF | 25 |
| 1193 | CASSSGGAETLYF | 25 |
| 1194 | CASSSGGYAEQFF | 25 |
| 1195 | CASSSGNQDTQYF | 25 |
| 1196 | CASSSGQGAETLYF | 25 |
| 1197 | CASSSGQGNTEVFF | 25 |
| 1198 | CASSSGSAETLYF | 25 |
| 1199 | CASSSGTGNTEVFF | 25 |
| 1200 | CASSSNERLFF | 25 |
| 1201 | CASSSNYAEQFF | 25 |
| 1202 | CASSSQANTEVFF | 25 |
| 1203 | CASSSQGSAETLYF | 25 |
| 1204 | CASSSTGGYAEQFF | 25 |
| 1205 | CASSTSQNTLYF | 25 |
| 1206 | CASSWDNTEVFF | 25 |
| 1207 | CASSWGNQDTQYF | 25 |
| 1208 | CAWSLGSQNTLYF | 25 |
| 1209 | CGARDSNYAEQFF | 25 |
| 1210 | CGARDWGYAEQFF | 25 |
| 1211 | CGARQGYAEQFF | 25 |
| 1212 | CSAGSQNTLYF | 25 |
| 1213 | CSASAETLYF | 25 |
| 1214 | CSASQNTLYF | 25 |
| 1215 | CASGDADQTYFQYF | 24 |
| 1216 | CASGDAEQYF | 24 |
| 1217 | CASGDAGGNYAEQFF | 24 |
| 1218 | CASGDAGSSYEQYF | 24 |
| 1219 | CASGDANTEVFF | 24 |
| 1220 | CASGDAPSQNTLYF | 24 |
| 1221 | CASGDDAETLYF | 24 |
| 1222 | CASGDGGNYAEQFF | 24 |
| 1223 | CASGDRANTEVFF | 24 |
| 1224 | CASGDRDQDTQYF | 24 |
| 1225 | CASGDRGAETLYF | 24 |
| 1226 | CASGDRNYAEQFF | 24 |
| 1227 | CASGDTNSDYTF | 24 |
| 1228 | CASGDVEQYF | 24 |
| 1229 | CASGDWDSAETLYF | 24 |
| 1230 | CASGDWGNTGQLYF | 24 |
| 1231 | CASGDWGQNTLYF | 24 |
| 1232 | CASGEGSQNTLYF | 24 |
| 1233 | CASGETDSGNTLYF | 24 |
| 1234 | CASGGTANSDYTF | 24 |
| 1235 | CASGTTNTEVFF | 24 |
| 1236 | CASRDRNYAEQFF | 24 |
| 1237 | CASRNTGQLYF | 24 |
| 1238 | CASRQGSQNTLYF | 24 |
| 1239 | CASRWDNYEQYF | 24 |
| 1240 | CASSDANSGNTLYF | 24 |
| 1241 | CASSDASQNTLYF | 24 |
| 1242 | CASSDDNSGNTLYF | 24 |
| 1243 | CASSDGDTGQLYF | 24 |
| 1244 | CASSDGGQNTLYF | 24 |
| 1245 | CASSDNNERLFF | 24 |
| 1246 | CASSDNNQAPLF | 24 |
| 1247 | CASSDQNTLYF | 24 |
| 1248 | CASSDRGNTEVFF | 24 |
| 1249 | CASSDRGSGNTLYF | 24 |
| 1250 | CASSDRNYAEQFF | 24 |
| 1251 | CASSDSGNTLYF | 24 |
| 1252 | CASSDSNERLFF | 24 |
| 1253 | CASSDWGNQDTQYF | 24 |
| 1254 | CASSDYAEQFF | 24 |
| 1255 | CASSEGAETLYF | 24 |
| 1256 | CASSFAETLYF | 24 |
| 1257 | CASSFIDWGNYAEQFF | 24 |
| 1258 | CASSFGGSQNTLYF | 24 |
| 1259 | CASSFNTGQLYF | 24 |
| 1260 | CASSFRAETLYF | 24 |
| 1261 | CASSFRGSQNTLYF | 24 |
| 1262 | CASSFRNTEVFF | 24 |
| 1263 | CASSFSGNILYF | 24 |
| 1264 | CASSGDNYAEQFF | 24 |
| 1265 | CASSGDSAETLYF | 24 |
| 1266 | CASSGDSSGNTLYF | 24 |
| 1267 | CASSGQGNSDYTF | 24 |
| 1268 | CASSGQSQNTLYF | 24 |
| 1269 | CASSGTAETLYF | 24 |
| 1270 | CASSGIGGAETLYF | 24 |
| 1271 | CASSGTGGNYAEQFF | 24 |
| 1272 | CASSGTGNYAEQFF | 24 |
| 1273 | CASSGTTNTEVFF | 24 |
| 1274 | CASSIGQNTEVFF | 24 |
| 1275 | CASSIRDNYAEQFF | 24 |
| 1276 | CASSISAETLYF | 24 |
| 1277 | CASSLAGANTEVFF | 24 |
| 1278 | CASSLAGNTLYF | 24 |
| 1279 | CASSLANTEVFF | 24 |
| 1280 | CASSLDGAETLYF | 24 |
| 1281 | CASSLDGQNTLYF | 24 |
| 1282 | CASSLDGSSAETLYF | 24 |
| 1283 | CASSLDKNTLYF | 24 |
| 1284 | CASSLDRDSDYTF | 24 |
| 1285 | CASSLDRNYAEQFF | 24 |
| 1286 | CASSLDSNERLFF | 24 |
| 1287 | CASSLDSNYAEQFF | 24 |
| 1288 | CASSLDSYNSPLYF | 24 |
| 1289 | CASSLDTNTEVFF | 24 |
| 1290 | CASSLEGDAEQFF | 24 |
| 1291 | CASSLEGGYAEQFF | 24 |
| 1292 | CASSLEGQQDTQYF | 24 |
| 1293 | CASSLEGTEVFF | 24 |
| 1294 | CASSLENSGNTLYF | 24 |
| 1295 | CASSLENYAEQFF | 24 |
| 1296 | CASSLEQTEVFF | 24 |
| 1297 | CASSLGDYAEQFF | 24 |
| 1298 | CASSLGENTEVFF | 24 |
| 1299 | CASSLGEVFF | 24 |
| 1300 | CASSLGGANTEVFF | 24 |
| 1301 | CASSLGGTGQLYF | 24 |
| 1302 | CASSLGLYAEQFF | 24 |
| 1303 | CASSLGNQAPLF | 24 |
| 1304 | CASSLGQGNSDYTF | 24 |
| 1305 | CASSLGTENTLYF | 24 |
| 1306 | CASSLGTGAETLYF | 24 |
| 1307 | CASSLGTGNTGQLYF | 24 |
| 1308 | CASSLGTNSDYTF | 24 |
| 1309 | CASSLGTSQNTLYF | 24 |
| 1310 | CASSLLGDYAEQFF | 24 |
| 1311 | CASSLLGNTLYF | 24 |
| 1312 | CASSLLGSQNTLYF | 24 |
| 1313 | CASSLNNQDTQYF | 24 |
| 1314 | CASSLNSGNTLYF | 24 |
| 1315 | CASSLQANTGQLYF | 24 |
| 1316 | CASSLQGDSDYTF | 24 |
| 1317 | CASSLQGDTEVFF | 24 |
| 1318 | CASSLQGETLYF | 24 |
| 1319 | CASSLQGGAEQFF | 24 |
| 1320 | CASSLQGSNERLFF | 24 |
| 1321 | CASSLQGTGQLYF | 24 |
| 1322 | CASSLQGYNSPLYF | 24 |
| 1323 | CASSLQNYAEQFF | 24 |
| 1324 | CASSLRGAETLYF | 24 |
| 1325 | CASSLRGDTEVFF | 24 |
| 1326 | CASSLRGNQDTQYF | 24 |
| 1327 | CASSLRGNTGQLYF | 24 |
| 1328 | CASSLRGNTLYF | 24 |
| 1329 | CASSLRGNYAEQFF | 24 |
| 1330 | CASSLRGSAETLYF | 24 |
| 1331 | CASSLSETLYF | 24 |
| 1332 | CASSLSGDQDTQYF | 24 |
| 1333 | CASSLSGNQDTQYF | 24 |
| 1334 | CASSLIGNSDYTF | 24 |
| 1335 | CASSLTISNERLFF | 24 |
| 1336 | CASSLINSDYTF | 24 |
| 1337 | CASSLTSSQNTLYF | 24 |
| 1338 | CASSLTVSNERLFF | 24 |
| 1339 | CASSLVAETLYF | 24 |
| 1340 | CASSLVGDQDTQYF | 24 |
| 1341 | CASSLVGGAETLYF | 24 |
| 1342 | CASSLVGGQNTLYF | 24 |
| 1343 | CASSLWGSAETLYF | 24 |
| 1344 | CASSNSGNTLYF | 24 |
| 1345 | CASSNSQNTLYF | 24 |
| 1346 | CASSPDRGQNTLYF | 24 |
| 1347 | CASSPDSNQDTQYF | 24 |
| 1348 | CASSPDSYAEQFF | 24 |
| 1349 | CASSPDWGAETLYF | 24 |
| 1350 | CASSPDWGSAETLYT | 24 |
| 1351 | CASSRDWGYAEQFF | 24 |
| 1352 | CASSPDYAEQFF | 24 |
| 1353 | CASSPGANTEVFF | 24 |
| 1354 | CASSPGLGAETLYF | 24 |
| 1355 | CASSPGLGENTLYF | 24 |
| 1356 | CASSPGLGYAEQFF | 24 |
| 1357 | CASSPGLNTGQLYF | 24 |
| 1358 | CASSPGLNYAEQFF | 24 |
| 1359 | CASSPGLQDTQYF | 24 |
| 1360 | CASSPGNYAEQFF | 24 |
| 1361 | CASSPGQNERLFF | 24 |
| 1362 | CASSPGQSAETLYF | 24 |
| 1363 | CASSPGTENTLYF | 24 |
| 1364 | CASSPGTGAETLYF | 24 |
| 1365 | CASSPGTSQNTLYF | 24 |
| 1366 | CASSPLGNYAEQFF | 24 |
| 1367 | CASSPNTEVFF | 24 |
| 1368 | CASSPQGSGNTLYF | 24 |
| 1369 | CASSPQNTLYF | 24 |
| 1370 | CASSPRAETLYF | 24 |
| 1371 | CASSPRDNYAEQFF | 24 |
| 1372 | CASSPTASAETLYF | 24 |
| 1373 | CASSPTGDQDTQYF | 24 |
| 1374 | CASSPTSQNTLYF | 24 |
| 1375 | CASSQANSDYTF | 24 |
| 1376 | CASSQDNYAEQFT | 24 |
| 1377 | CASSQDSGNTLYF | 24 |
| 1378 | CASSQEGAETLYF | 24 |
| 1379 | CASSQRDWCYAEQFF | 24 |
| 1380 | CASSQSQNTLYF | 24 |
| 1381 | CASSRANTGQLYF | 24 |
| 1382 | CASSRDLSAETLYF | 24 |
| 1383 | CASSRDNNNQAPLF | 24 |
| 1384 | CASSRDRAETLYF | 24 |
| 1385 | CASSRDRGAETLYF | 24 |
| 1386 | CASSRDRGNSDYTF | 24 |
| 1387 | CASSRDRGQNTLYF | 24 |
| 1388 | CASSRDRGYAEQFF | 24 |
| 1389 | CASSRDSGNTLYF | 24 |
| 1390 | CASSRDTYAEQFF | 24 |
| 1391 | CASSRDWGNQDTQYF | 24 |
| 1392 | CASSRGQNYAEQFF | 24 |
| 1393 | CASSRLGDQDTQYF | 24 |
| 1394 | CASSRLGDYAEQFF | 24 |
| 1395 | CASSRQDQDTQYF | 24 |
| 1396 | CASSRQGDTGQLYF | 24 |
| 1397 | CASSRQGSAETLYF | 24 |
| 1398 | CASSRQGSGNTLYF | 24 |
| 1399 | CASSRQGTEVFF | 24 |
| 1400 | CASSRQUSNERLFF | 24 |
| 1401 | CASSRQNTLYF | 24 |
| 1402 | CASSRQSNTEVFF | 24 |
| 1403 | CASSRQTNTEVFF | 24 |
| 1404 | CASSRSAETLYF | 24 |
| 1405 | CASSRTANTEVFF | 24 |
| 1406 | CASSRTENTLYF | 24 |
| 1407 | CASSRTGAETLYF | 24 |
| 1408 | CASSRTGGNYAEQFF | 24 |
| 1409 | CASSRTGGSQNTLYF | 24 |
| 1410 | CASSRTGNYAEQFF | 24 |
| 1411 | CASSRTSAETLYF | 24 |
| 1412 | CASSSDAEQFF | 24 |
| 1413 | CASSSGQNTGQLYF | 24 |
| 1414 | CASSSGIGNERLFF | 24 |
| 1415 | CASSSNSGNTLYF | 24 |
| 1416 | CASSSNTEVFF | 24 |
| 1417 | CASSSQGSGNTLYF | 24 |
| 1418 | CASSSQGSQNTLYF | 24 |
| 1419 | CASSSQGTEVFF | 24 |
| 1420 | CASSSSSAETLYF | 24 |
| 1421 | CASSSSSQNTLYF | 24 |
| 1422 | CASSSTANTGQLYF | 24 |
| 1423 | CASSSTGNTGQLYF | 24 |
| 1424 | CASSSTSQNTLYF | 24 |
| 1425 | CASSTGDQDTQYF | 24 |
| 1426 | CASSTGGAETLYT | 24 |
| 1427 | CASSTGGQNTLYF | 24 |
| 1428 | CASSTGGYAEQFF | 24 |
| 1429 | CASSTGNTEVFF | 24 |
| 1430 | CASSTGSQNTLYF | 24 |
| 1431 | CASSTNTGQLAT | 24 |
| 1432 | CASSWDRNTEVFF | 24 |
| 1433 | CGARDHTSNTEVFF | 24 |
| 1434 | CGARDWGNTGQLYF | 24 |
| 1435 | CGARDWGSQNTLYF | 24 |
| 1436 | CSADSQNTLYF | 24 |
| 1437 | CASGDAGANTEVFF | 23 |
| 1438 | CASGDAGGNTEVFF | 23 |
| 1439 | CASGDAGGNTGQLYF | 23 |
| 1440 | CASGDAGGSQNTLYF | 23 |
| 1441 | CASGDAGSGNTLYF | 23 |
| 1442 | CASGDAGTANTEVFF | 23 |
| 1443 | CASGDAGVQDTQYF | 23 |
| 1444 | CASGDAQDTQYF | 23 |
| 1445 | CASGDAQSQNTLYF | 23 |
| 1446 | CASGDASSGNTLYF | 23 |
| 1447 | CASGDATISAETLYF | 23 |
| 1448 | CASGDAYNSPLYF | 23 |
| 1449 | CASGDGGNQDTQYF | 23 |
| 1450 | CASGDGNTEVFF | 23 |
| 1451 | CASGDGTTNTEVFF | 23 |
| 1452 | CASGDNQAPLF | 23 |
| 1453 | CASGDNQDTQYF | 23 |
| 1454 | CASGDNSAETLYF | 23 |
| 1455 | CASGDQNTLYF | 23 |
| 1456 | CASGDRANSDYTF | 23 |
| 1457 | CASGDRDTEVFF | 23 |
| 1458 | CASGDRGNYAEQFF | 23 |
| 1459 | CASGDRNSDYTF | 23 |
| 1460 | CASGDSYNSPLYF | 23 |
| 1461 | CASGDVGSQNTLYF | 23 |
| 1462 | CASGDWGAETLYF | 23 |
| 1463 | CASGDWGGYAEQFF | 23 |
| 1464 | CASGDWGQDTQYF | 23 |
| 1465 | CASGEQDTQYF | 23 |
| 1466 | CASGESQNTLYF | 23 |
| 1467 | CASGETANTEVFF | 23 |
| 1468 | CASGNQDTQYF | 23 |
| 1469 | CASGTGNYAEQFF | 23 |
| 1470 | CASRDSGNTLYF | 23 |
| 1471 | CASRDSSAETLYF | 23 |
| 1472 | CASRSAETLYF | 23 |
| 1473 | CASSAGSQNTLYF | 23 |
| 1474 | CASSANTGQLYF | 23 |
| 1475 | CASSDAETLYF | 23 |
| 1476 | CASSDAGAETLYF | 23 |
| 1477 | CASSDAGNTEVFF | 23 |
| 1478 | CASSDASSGNTLYF | 23 |
| 1479 | CASSDGSAETLYF | 23 |
| 1480 | CASSDNERLFF | 23 |
| 1481 | CASSDNSDYTF | 23 |
| 1482 | CASSDNIGQLYF | 23 |
| 1483 | CASSDRANTEVFF | 23 |
| 1484 | CASSDRDSGNTLYF | 23 |
| 1485 | CASSDRNERLFF | 23 |
| 1486 | CASSDRSQNTLYF | 23 |
| 1487 | CASSDSSYNSPLYF | 23 |
| 1488 | CASSDTGQLYF | 23 |
| 1489 | CASSDWGQDTQYF | 23 |
| 1490 | CASSDWGSQNTLYF | 23 |
| 1491 | CASSEQGNTEVFF | 23 |
| 1492 | CASSESAETLYF | 23 |
| 1493 | CASSFDTGQLYF | 23 |
| 1494 | CASSFGQNTEVFF | 23 |
| 1495 | CASSFNSAETLYF | 23 |
| 1496 | CASSTQNTEVFF | 23 |
| 1497 | CASSFSNERLFF | 23 |
| 1498 | CASSFTGGQNTLYF | 23 |
| 1499 | CASSFWGNYAEQFF | 23 |
| 1500 | CASSGDWGNYAEQFF | 23 |
| 1501 | CASSGQNTEVFF | 23 |
| 1502 | CASSGTGGQDTQYF | 23 |
| 1503 | CASSGTNTEVFF | 23 |
| 1504 | CASSHNQDTQYF | 23 |
| 1505 | CASSINSAETLYF | 23 |
| 1506 | CASSIRGNTEVFF | 23 |
| 1507 | CASSLAGGQNTLYF | 23 |
| 1508 | CASSLAGNSDYTF | 23 |
| 1509 | CASSLDGNQDTQYF | 23 |
| 1510 | CASSLDGNYAEQFF | 23 |
| 1511 | CASSLDNQAPLF | 23 |
| 1512 | CASSLDSANTEVFF | 23 |
| 1513 | CASSLDSTEVFF | 23 |
| 1514 | CASSLDWGDAEQFF | 23 |
| 1515 | CASSLEAETLYF | 23 |
| 1516 | CASSLEGGNYAEQFF | 23 |
| 1517 | CASSLEQNTEVFF | 23 |
| 1518 | CASSLGAQNTLYF | 23 |
| 1519 | CASSLGASAETLYF | 23 |
| 1520 | CASSLGDNQDTQYF | 23 |
| 1521 | CASSLGDTLYF | 23 |
| 1522 | CASSLGGAGNTLYF | 23 |
| 1523 | CASSLGGGAETLYF | 23 |
| 1524 | CASSLGHYAEQFF | 23 |
| 1525 | CASSLGLGENTLYF | 23 |
| 1526 | CASSLGLSAETLYF | 23 |
| 1527 | CASSLGNERLFF | 23 |
| 1528 | CASSLGQANTGQLYF | 23 |
| 1529 | CASSLGQGAGNTLYF | 23 |
| 1530 | CASSLGQGNIGQLYF | 23 |
| 1531 | CASSLGQGNYAEQFF | 23 |
| 1532 | CASSLGQGQNTLYF | 23 |
| 1533 | CASSLGQNQAPLF | 23 |
| 1534 | CASSLGUNSPLYF | 23 |
| 1535 | CASSLGRNTEVFF | 23 |
| 1536 | CASSLGSSGNTLYF | 23 |
| 1537 | CASSLGTGGAETLYF | 23 |
| 1538 | CASSLGTGNTEVFF | 23 |
| 1539 | CASSLGTTNERLFF | 23 |
| 1540 | CASSLLGNQDTQYF | 23 |
| 1541 | CASSLIGNTGQLYF | 23 |
| 1542 | CASSUGSAETLYF | 23 |
| 1543 | CASSLLGTSAETLYF | 23 |
| 1544 | CASSLNNYAEQFF | 23 |
| 1545 | CASSLNYAEQFF | 23 |
| 1546 | CASSLQENTLYF | 23 |
| 1547 | CASSLQGANERLFF | 23 |
| 1548 | CASSLQGDTGQLYF | 23 |
| 1549 | CASSLQGGAETLYF | 23 |
| 1550 | CASSLQGGQNTLYF | 23 |
| 1551 | CASSLQGGTEVFF | 23 |
| 1552 | CASSLQGGYAEQFF | 23 |
| 1553 | CASSLQGNQAPLF | 23 |
| 1554 | CASSLQGTNERLFF | 23 |
| 1555 | CASSLRANTEVFF | 23 |
| 1556 | CASSLRDNYAEQFF | 23 |
| 1557 | CASSLRDSGNTLYF | 23 |
| 1558 | CASSIAGGQNTLYF | 23 |
| 1559 | CASSLRGNSDYTF | 23 |
| 1560 | CASSLSANSDYTF | 23 |
| 1561 | CASSLSANTEVFF | 23 |
| 1562 | CASSLSGTEVFF | 23 |
| 1563 | CASSLSNSGNTLYF | 23 |
| 1564 | CASSLTGANSDYTF | 23 |
| 1565 | CASSLTGDSDYTF | 23 |
| 1566 | CASSLTGDTEVFF | 23 |
| 1567 | CASSLTGDTGQLYF | 23 |
| 1568 | CASSLTGGNYAEQFF | 23 |
| 1569 | CASSLTGNQDTQYF | 23 |
| 1570 | CASSLTGYAEQFF | 23 |
| 1571 | CASSLVGNTGQLYF | 23 |
| 1572 | CASSLVGQNTLYF | 23 |
| 1573 | CASSLVTGQLYF | 23 |
| 1574 | CASSPDTEVFF | 23 |
| 1575 | CASSPGANTGQLYF | 23 |
| 1576 | CASSPGGSAETLYF | 23 |
| 1577 | CASSPGGYAEQFF | 23 |
| 1578 | CASSPGLGQNTLYF | 23 |
| 1579 | CASSPGLGSQNTLYF | 23 |
| 1580 | CASSPGNQDTQYF | 23 |
| 1581 | CASSPGNTGQLYF | 23 |
| 1582 | CASSPGQGNERLFF | 23 |
| 1583 | CASSPGQKNTLYF | 23 |
| 1584 | CASSPGQNQAPLF | 23 |
| 1585 | CASSPGTANERLFF | 23 |
| 1586 | CASSPGTDTEVFF | 23 |
| 1587 | CASSPNSAETLYF | 23 |
| 1588 | CASSPNSQNTLYF | 23 |
| 1589 | CASSPQGANTEVFF | 23 |
| 1590 | CASSPQGNSDYTF | 23 |
| 1591 | CASSPRGAETLYF | 23 |
| 1592 | CASSPSSQNTLYF | 23 |
| 1593 | CASSPTGGAETLYF | 23 |
| 1594 | CASSPTGSAETLYF | 23 |
| 1595 | CASSPTINQDTQYF | 23 |
| 1596 | CASSPTTNTEVFF | 23 |
| 1597 | CASSPTVNQDTQYF | 23 |
| 1598 | CASSQANTEVFF | 23 |
| 1599 | CASSQDGSQNTLYF | 23 |
| 1600 | CASSQDNSGNTLYF | 23 |
| 1601 | CASSQDRGSAETLYF | 23 |
| 1602 | CASSQDSYNSPLYF | 23 |
| 1603 | CASSQDWGQNTLYF | 23 |
| 1604 | CASSQGANSDYTF | 23 |
| 1605 | CASSQGANTEVFF | 23 |
| 1606 | CASSQGNTEVFF | 23 |
| 1607 | CASSQGNTLYF | 23 |
| 1608 | CASSQGTANSDYTF | 23 |
| 1609 | CASSQQGSAETLYF | 23 |
| 1610 | CASSQQGTEVFF | 23 |
| 1611 | CASSQTANSDYTF | 23 |
| 1612 | CASSQTGGQNTLYF | 23 |
| 1613 | CASSRDKNTGQLYF | 23 |
| 1614 | CASSRDNNQAPLF | 23 |
| 1615 | CASSRDNNQDTQNYF | 23 |
| 1616 | CASSRDRYAEQFF | 23 |
| 1617 | CASSRDSSNERLFF | 23 |
| 1618 | CASSRDTNTEVFF | 23 |
| 1619 | CASSRDTQDTQYF | 23 |
| 1620 | CASSRDWGDTQYF | 23 |
| 1621 | CASSRDWGSYEQYF | 23 |
| 1622 | CASSRDWNYAEQFF | 23 |
| 1623 | CASSRDWSAETLYF | 23 |
| 1624 | CASSRDYAEQFF | 23 |
| 1625 | CASSRGNTEVFF | 23 |
| 1626 | CASSRGQNTEVFF | 23 |
| 1627 | CASSRLGANTGQLYF | 23 |
| 1628 | CASSRLGENTLYF | 23 |
| 1629 | CASSRLGSSAETLYF | 23 |
| 1630 | CASSRQANTGQLYF | 23 |
| 1631 | CASSRQGETLYF | 23 |
| 1632 | CASSRQGEVFF | 23 |
| 1633 | CASSRQGYAEQFF | 23 |
| 1634 | CASSRQQNTLYF | 23 |
| 1635 | CASSRQSAETLYF | 23 |
| 1636 | CASSRQYAEQFF | 23 |
| 1637 | CASSRTASQNTLYF | 23 |
| 1638 | CASSRTGGNTLYF | 23 |
| 1639 | CASSRTGNTEVFF | 23 |
| 1640 | CASSRTGNTLYF | 23 |
| 1641 | CASSRTISNERLFF | 23 |
| 1642 | CASSRTTSAAETLYF | 23 |
| 1643 | CASSSANTEVFF | 23 |
| 1644 | CASSSDSSQNTLYF | 23 |
| 1645 | CASSSDWGQNTLYF | 23 |
| 1646 | CASSSGANTEVFF | 23 |
| 1647 | CASSSGGTEVFF | 23 |
| 1648 | CASSSGQNYAEQFF | 23 |
| 1649 | CASSSGTANSDYTF | 23 |
| 1650 | CASSSGTTNSDYTF | 23 |
| 1651 | CASSSQGAGNTLYF | 23 |
| 1652 | CASSSQGYAEQFF | 23 |
| 1653 | CASSSTGDTGQLYF | 23 |
| 1654 | CASSSTGNSDYTF | 23 |
| 1655 | CASSTGGNYAEQFF | 23 |
| 1656 | CASSTGTANTEVFF | 23 |
| 1657 | CASSVNQDTQYF | 23 |
| 1658 | CASSVSAETLYF | 23 |
| 1659 | CASSWGDQDTQYF | 23 |
| 1660 | CASSWTDSGNTLYF | 23 |
| 1661 | CAWSLGDQDTQYF | 23 |
| 1662 | CAWSLGGNYAEQFF | 23 |
| 1663 | CAWSLGGQDTQYF | 23 |
| 1664 | CAWSLGSAETLYF | 23 |
| 1665 | CAWSLSAETLYF | 23 |
| 1666 | CGARDNYAEQFF | 23 |
| 1667 | CGARDRNTGQLYF | 23 |
| 1668 | CGARDSQNTLYF | 23 |
| 1669 | CGARDWGSAETLYF | 23 |
| 1670 | CSADTEVFF | 23 |
| 1671 | CASGDAANSDYTF | 22 |
| 1672 | CASGDAGEDTQYF | 22 |
| 1673 | CASGDAGGEQYF | 22 |
| 1674 | CASGDAGGNQDTQYF | 22 |
| 1675 | CASGDAGISNERLFF | 22 |
| 1676 | CASGDAGQNSDYTF | 22 |
| 1677 | CASGDAGVSYEQYF | 22 |
| 1678 | CASGDARYEQYF | 22 |
| 1679 | CASGDAWGQDTQYF | 22 |
| 1680 | CASGDDRGQNTLYF | 22 |
| 1681 | CASGDGGNTEVFF | 22 |
| 1682 | CASGDGGSQNTLYF | 22 |
| 1683 | CASGDGSAETLYF | 22 |
| 1684 | CASGDGTANSDYTF | 22 |
| 1685 | CASGDLGNYAEQFF | 22 |
| 1686 | CASGDNNERLFF | 22 |
| 1687 | CASGDNSDYTF | 22 |
| 1688 | CASGDPANSDYTF | 22 |
| 1689 | CASGDRDNYAEQFF | 22 |
| 1690 | CASGDRGNTGQLYF | 22 |
| 1691 | CASGDRGQDTQYF | 22 |
| 1692 | CASGDSANTEVFF | 22 |
| 1693 | CASGDTSAETLYF | 22 |
| 1694 | CASGEEDTQYF | 22 |
| 1695 | CASGETGNTEVFF | 22 |
| 1696 | CASGETGNYAEQFF | 22 |
| 1697 | CASGETLYF | 22 |
| 1698 | CASGPGQGYAEQFF | 22 |
| 1699 | CASGSQNTLYF | 22 |
| 1700 | CASGTANTEVFF | 22 |
| 1701 | CASGTGNSDYTF | 22 |
| 1702 | CASRDNANTEVFF | 22 |
| 1703 | CASRDNQDTQYF | 22 |
| 1704 | CASRGQGNTEVFF | 22 |
| 1705 | CASRQGANTEVFF | 22 |
| 1706 | CASRQNTEVFF | 22 |
| 1707 | CASRSQNTLYF | 22 |
| 1708 | CASSADSAETLYF | 22 |
| 1709 | CASSAGTANTEVFF | 22 |
| 1710 | CASSDAGSNETLYF | 22 |
| 1711 | CASSDGGNTEVFF | 22 |
| 1712 | CASSDGTANSDYTF | 22 |
| 1713 | CASSDNSAETLYF | 22 |
| 1714 | CASSDPGQDTQYF | 22 |
| 1715 | CASSDRDQDTQYF | 22 |
| 1716 | CASSDRETLYF | 22 |
| 1717 | CASSDRGQDTQYF | 22 |
| 1718 | CASSDRGSAETLYF | 22 |
| 1719 | CASSDTANTEVFF | 22 |
| 1720 | CASSEDTEVFF | 22 |
| 1721 | CASSEGAGNTLYF | 22 |
| 1722 | CASSFDNSGNTLYF | 22 |
| 1723 | CASSFDRNTEVFF | 22 |
| 1724 | CASSFGERLFF | 22 |
| 1725 | CASSFLGNYAEQFF | 22 |
| 1726 | CASSENERLFF | 22 |
| 1727 | CASSFQANSDYTF | 22 |
| 1728 | CASSFQNYAEQFF | 22 |
| 1729 | CASSFRTENTEVFF | 22 |
| 1730 | CASSFSSGNTLYF | 22 |
| 1731 | CASSFEANTEVFF | 22 |
| 1732 | CASSGGAETLYF | 22 |
| 1733 | CASSGNTLYF | 22 |
| 1734 | CASSGQNIGQLYF | 22 |
| 1735 | CASSGTDYAEQFF | 22 |
| 1736 | CASSGTGGNTLYF | 22 |
| 1737 | CASSGTGNTLYF | 22 |
| 1738 | CASSGTSAETLYF | 22 |
| 1739 | CASSGTSSAETLYF | 22 |
| 1740 | CASSGTTSAETLYF | 22 |
| 1741 | CASSHNYAEQFF | 22 |
| 1742 | CASSIGAETLYF | 22 |
| 1743 | CASSIGGNYAEQFF | 22 |
| 1744 | CASSLAGGAETLYF | 22 |
| 1745 | CASSLAQNTEVEF | 22 |
| 1746 | CASSLAQNTLYF | 22 |
| 1747 | CASSLASSGNTLYF | 22 |
| 1748 | CASSLDGGSAETLYF | 22 |
| 1749 | CASSLDGNSDYTF | 22 |
| 1750 | CASSLDGNTGQLYF | 22 |
| 1751 | CASSLDGSAETLYF | 22 |
| 1752 | CASSLDKEVFF | 22 |
| 1753 | CASSLDNNERLFF | 22 |
| 1754 | CASSLDNNQAPLF | 22 |
| 1755 | CASSLDRANTEVFF | 22 |
| 1756 | CASSLDRGQNTLYF | 22 |
| 1757 | CASSLDRGYAEQFF | 22 |
| 1758 | CASSLDSNTGQLYF | 22 |
| 1759 | CASSLDSSNERLFF | 22 |
| 1760 | CASSLDWGAETLYF | 22 |
| 1761 | CASSLEETLYF | 22 |
| 1762 | CASSLEGAQDTQYF | 22 |
| 1763 | CASSLEGASQNTLYF | 22 |
| 1764 | CASSLEGGAETLYF | 22 |
| 1765 | CASSLEGGNTLYF | 22 |
| 1766 | CASSLEGNSGNTLYF | 22 |
| 1767 | CASSLEGRQNTLYF | 22 |
| 1768 | CASSLEGTGNTLYF | 22 |
| 1769 | CASSLEQGAETLYF | 22 |
| 1770 | CASSLEQNTGQLYF | 22 |
| 1771 | CASSLEQYAEQFF | 22 |
| 1772 | CASSLGGENTLYF | 22 |
| 1773 | CASSLGGETLYF | 22 |
| 1774 | CASSLGGRAETLYF | 22 |
| 1775 | CASSLGHTEVFF | 22 |
| 1776 | CASSLGLGNYAEQFF | 22 |
| 1777 | CASSLGLGQNTLYF | 22 |
| 1778 | CASSLGLNQDTQYF | 22 |
| 1779 | CASSLGLNYAEQFF | 22 |
| 1780 | CASSLGLSQNTLYF | 22 |
| 1781 | CASSLGPNTEVFF | 22 |
| 1782 | CASSLGQGNERLFF | 22 |
| 1783 | CASSLGQNERLFF | 22 |
| 1784 | CASSLGSQDTQYF | 22 |
| 1785 | CASSLGTAETLYF | 22 |
| 1786 | CASSLGTDYAEQFF | 22 |
| 1787 | CASSLGTGYAEQFF | 22 |
| 1788 | CASSLGTNQDTQYF | 22 |
| 1789 | CASSLGTTSAETLYF | 22 |
| 1790 | CASSLGTYAEQFF | 22 |
| 1791 | CASSLLGAETLYF | 22 |
| 1792 | CASSLLGENTLYF | 22 |
| 1793 | CASSLLGGQNTLYF | 22 |
| 1794 | CASSLNAETLYF | 22 |
| 1795 | CASSLNNERLFF | 22 |
| 1796 | CASSLNTLYF | 22 |
| 1797 | CASSLPGSQNTLYF | 22 |
| 1798 | CASSLQGERLFF | 22 |
| 1799 | CASSLQNSGNTLYF | 22 |
| 1800 | CASSLQYAEQFF | 22 |
| 1801 | CASSLRANTGQLYF | 22 |
| 1802 | CASSLRDTLYF | 22 |
| 1803 | CASSLRGYAEQFF | 22 |
| 1804 | CASSLRNSGNTLYF | 22 |
| 1805 | CASSLRNTEVFF | 22 |
| 1806 | CASSLRQNTLYF | 22 |
| 1807 | CASSLSGNSDYTF | 22 |
| 1808 | CASSLSNSDYTF | 22 |
| 1809 | CASSLSTNSDYTF | 22 |
| 1810 | CASSLSVNQDTQYF | 22 |
| 1811 | CASSLTGSGNTLYF | 22 |
| 1812 | CASSLTGTEVFF | 22 |
| 1813 | CASSLTPNTEVFF | 22 |
| 1814 | CASSLTTNTGQLYF | 22 |
| 1815 | CASSLVANSDYTF | 22 |
| 1816 | CASSLVGSAETLYF | 22 |
| 1817 | CASSLVNSGNTLYF | 22 |
| 1818 | CASSLVQNTLYF | 22 |
| 1819 | CASSPANTGQLYF | 22 |
| 1820 | CASSPDNSQNTLYF | 22 |
| 1821 | CASSPDTSQNTLYF | 22 |
| 1822 | CASSPGDTEVFF | 22 |
| 1823 | CASSPGHQDTQYF | 22 |
| 1824 | CASSPGLGSAETLYF | 22 |
| 1825 | CASSPGLSQNTLYF | 22 |
| 1826 | CASSPGQGNTLYF | 22 |
| 1827 | CASSPGQISNERLFF | 22 |
| 1828 | CASSPGQNNQAPLF | 22 |
| 1829 | CASSPGTGNSDYTF | 22 |
| 1830 | CASSPGTGNTEVFF | 22 |
| 1831 | CASSPQGDTGQLYF | 22 |
| 1832 | CASSPQGNYAEQFF | 22 |
| 1833 | CASSPQNTEVFF | 22 |
| 1834 | CASSPRDSAETLYF | 22 |
| 1835 | CASSPRLGQDTQYF | 22 |
| 1836 | CASSPSGNTLYF | 22 |
| 1837 | CASSPTGNTEVFF | 22 |
| 1838 | CASSPTGYAEQFF | 22 |
| 1839 | CASSPTISNERLFF | 22 |
| 1840 | CASSPTNSGNTLYF | 22 |
| 1841 | CASSPTNTEVFF | 22 |
| 1842 | CASSPWGNYAEQFF | 22 |
| 1843 | CASSQDRANTEVFF | 22 |
| 1844 | CASSQDSNQDTQYF | 22 |
| 1845 | CASSQDWDQDTQYF | 22 |
| 1846 | CASSQDWGSAFTLYF | 22 |
| 1847 | CASSQEGNQDTQYF | 22 |
| 1848 | CASSQEGNTEVFF | 22 |
| 1849 | CASSQEGQQDTQYF | 22 |
| 1850 | CASSQGNSGNTLYF | 22 |
| 1851 | CASSQGQQNTLYF | 22 |
| 1852 | CASSQGTASAETLYF | 22 |
| 1853 | CASSQGTISNERLFF | 22 |
| 1854 | CASSQNYAEQFF | 22 |
| 1855 | CASSQQGAGNTLYF | 22 |
| 1856 | CASSQTGNTGQLYF | 22 |
| 1857 | CASSRDINQDTQYF | 22 |
| 1858 | CASSRDISGNTLYF | 22 |
| 1859 | CASSRDKNTEVFF | 22 |
| 1860 | CASSRDNANSDYTF | 22 |
| 1861 | CASSRDRGNTEVFF | 22 |
| 1862 | CASSRDRGTEVFF | 22 |
| 1863 | CASSRDRSQNTLYF | 22 |
| 1864 | CASSRDSNTEVFF | 22 |
| 1865 | CASSRDTNYAEQFF | 22 |
| 1866 | CASSRDWEDTQYF | 22 |
| 1867 | CASSRDWGAETLYF | 22 |
| 1868 | CASSRDWGQNTLYF | 22 |
| 1869 | CASSRGDQDTQYF | 22 |
| 1870 | CASSRGGQNTLYF | 22 |
| 1871 | CASSRGQGYAEQFF | 22 |
| 1872 | CASSRNYAEQFF | 22 |
| 1873 | CASSRQGDSDYTF | 22 |
| 1874 | CASSRQNQAPLF | 22 |
| 1875 | CASSRTAETLYF | 22 |
| 1876 | CASSRTGGNTEVFF | 22 |
| 1877 | CASSSIDSSGNTLYF | 22 |
| 1878 | CASSSGENTLYF | 22 |
| 1879 | CASSSGGNYAEQFF | 22 |
| 1880 | CASSSGGSAETLYF | 22 |
| 1881 | CASSSGGSQNTLYF | 22 |
| 1882 | CASSSGLGQDTQYF | 22 |
| 1883 | CASSSGNTGQLYF | 22 |
| 1884 | CASSSGQKNTLYF | 22 |
| 1885 | CASSSGQSQNTLYF | 22 |
| 1886 | CASSSGQYAEQFF | 22 |
| 1887 | CASSSGSSAETLYF | 22 |
| 1888 | CASSSGTGAETLYF | 22 |
| 1889 | CASSSLGDQDTQYF | 22 |
| 1890 | CASSSNSAETLYF | 22 |
| 1891 | CASSSNSDYTF | 22 |
| 1892 | CASSSNSQNTLYF | 22 |
| 1893 | CASSSQGNQDTQYF | 22 |
| 1894 | CASSSQNTEVFF | 22 |
| 1895 | CASSSQNYAEQFF | 22 |
| 1896 | CASSSTGSQNTLYF | 22 |
| 1897 | CASSSTNSDYTF | 22 |
| 1898 | CASSSWGSQNTLYF | 22 |
| 1899 | CASSTGNYAEQFF | 22 |
| 1900 | CASSIGSAETLYF | 22 |
| 1901 | CASSWGGQNTLYF | 22 |
| 1902 | CASSWTANTGQLYF | 22 |
| 1903 | CASSYGAETLYF | 22 |
| 1904 | CASSYGGQNTLYF | 22 |
| 1905 | CASSYNNQAPLF | 22 |
| 1906 | CASSYQANTEVFF | 22 |
| 1907 | CASSYRNTEVFF | 22 |
| 1908 | CASSYWGNYAEQFF | 22 |
| 1909 | CAWSLGGQNTLYF | 22 |
| 1910 | CAWSLGNYAEQFF | 22 |
| 1911 | CAWSLGSSAETLYF | 22 |
| 1912 | CAWSLGYEQYF | 22 |
| 1913 | CGARDNSGNTLYF | 22 |
| 1914 | CGARDSSQNTLYF | 22 |
| 1915 | CGARDSSYNSPLYF | 22 |
| 1916 | CGARDWGSSYEQYF | 22 |
| 1917 | CGARQGNTEVFF | 22 |
| 1918 | CGARTVSNERLFF | 22 |
| 1919 | CSADAEQFF | 22 |
| 1920 | CSAGNSDYTF | 22 |
| 1921 | CSAGQNTEVFF | 22 |
| 1922 | CSAGSAETLYF | 22 |
| 1923 | CASSQSRYEQYF | |
| 1924 | CASSLGYINTGQLYF | |

Each possibility represents a separate embodiment of the present invention.

According to some embodiments of the present invention, the CDR3 sequence is selected from the group consisting of the sequences in Table 9.

According to some embodiments of the present invention, the peptide amino acid sequence is selected from the group consisting of SEQ ID NOs of Table 9.

TABLE 9

| SEQ | | SEQ | | SEQ | |
|---|---|---|---|---|---|
| ID | | ID | | ID | |
| NO: | Sequence | NO: | Sequence | NO: | Sequence |
| 21 | CASSLGNSDYTF | 40 | CASSLQGYEQYF | 77 | CASSLDSYEQYF |
| 23 | CASSSGNSDYTF | 41 | CASSDSSYEQYF | 78 | CASSRQYEQYF |
| 22 | CASSSANSDYTE | 42 | CASSLGSSYEQYF | 79 | CASSLKDTQYF |
| 5 | CASGGYEQYF | 44 | CASGDYEQYF | 81 | CASSFGTGDEQYF |
| 1924 | CASSLGTTNTGQLYF | 45 | CASSLEDTQYF | 82 | CASSLGEDTQYF |
| 27 | CASGDAGGSYEQYF | 46 | CASSLEGDEQYF | 84 | CASSPSSYEQYF |
| 30 | CASSGTDQDTQYF | 47 | CASSLGYEQYF | 85 | CASSPDSYEQYF |
| 32 | CASSRTANTGQLYF | 48 | CASSLGQYEQYF | 86 | CASSRDNYEQYF |
| 20 | CASSLGGQNTLYF | 49 | CASSVDGSYEQYF | 87 | CASSRDSYEQYF |
| 25 | CASSGQGNYAEQFF | 50 | CASSPQDTQYF | 88 | CASSLDRVEQYF |
| 12 | CASSFGTGYEQYF | 51 | CASSLDNYEQYF | 90 | CASSLDAEQFF |
| 31 | CASSPGQSNERLFF | 52 | CASSLEGYEQYF | 91 | CASSDSYEQYF |
| 34 | CASSLEGDTEVEF | 53 | CASSLDEQYF | 93 | CASSPDNYEQYF |
| 6 | CASSPGGSYEQYF | 55 | CASSLEGDTQYF | 94 | CASSPGQYEQYF |
| 10 | CASSRGSYEQYF | 56 | CASSLDYEQYF | 95 | CASSLQDTQYF |
| 1923 | CASSQSRYEQYF | 57 | CASSLGDTQYF | 96 | CASSLRDTQYF |
| 29 | CASSHSGNTLYF | 58 | CASSLGEQYF | 97 | CASSLGDEQYF |
| 24 | CASSGTANTEVFF | 60 | CASSLDRYEQYF | 98 | CASSLVAEQFF |
| 19 | CASSRRPYEQYF | 61 | CASSGTGGYEQYF | 99 | CASSFSYEQYF |
| 8 | CASSGTGKDTQYF | 62 | CASSSYEQYF | 100 | CASSLSYEQYF |
| 1 | CASSGTGEDTQYF | 65 | CASSLDRDEQYF | 102 | CASSDAGDTQYF |
| 2 | CASSGTGQDTQYF | 67 | CASSLEGSSYEQYF | 103 | CASGDSYEQYF |
| 4 | CASSGLGEDTQYF | 68 | CASSLGGYEQYF | 104 | CASSLDTQYF |
| 16 | CASSGRGQDTQYF | 69 | CASSLGAEQFF | 106 | CASGDEQYF |
| 15 | CASTGTGQDTQYF | 70 | CASSLSGYEQYF | 108 | CASSLYEQYF |
| 17 | CASGGAYEQYF | 71 | CASSSSYEQYF | 109 | CASSRDSSYEQYF |
| 37 | CASSTQDTQYF | 72 | CASSLGQDTQYF | 110 | CASSIRDTQYF |
| 38 | CASSRQQDTQYF | 73 | CASSLGGQDTQYF | | |

Each possibility represents a separate embodiment of the present invention.

Accordingly, the peptide may have a sequence which encompasses the entire CDR3 sequences as presented in any one of the tables above.

It will be appreciated that peptide fragments are also encompassed according to the present teachings, as long as the peptide maintains its function e.g., capable of competing with binding of a TCR having the respective CDR3 sequence presented on a T cell to a target of the T cell. According to specific embodiments, the peptide may be 6-20, 8-20, 10-20, 15-20, 6-16, 8-16, 10-16, 12-16, 6-14, 8-14, 10-14 amino acids long.

According to some embodiments, the isolated peptide comprises a sequence selected from the group consisting of SEQ ID NOs: 2-6, 8, 10, 12, 15-17, 19-25, 27, 29-32, 34, 37, 38, 40-42, 44-53, 55-58, 60-62, 65, 67-73, 77-79, 81, 82, 84-88, 90, 91, 93-100, 102-104, 106, 108-110, 1923 and 1924.

Each possibility represents a separate embodiment of the present invention.

It should be noted that any known peptide, such as the peptide ASSLGGNQDTQY (denoted C-9, SEQ ID NO: 1), is excluded from the scope of isolated peptides per se of the present invention.

According to some specific embodiments, the peptide is selected from the group consisting of SEQ ID NOs: 2-6, 8, 10, 12, 15-17, 19-25, 27, 29-32, 34, 1923 and 1924.

Each possibility represents a separate embodiment of the present invention. According to yet other embodiments, the peptide is selected from the group consisting of SEQ ID NOs: 3, 6, 8, 10, 12, 15-17, 19, 27 and 1923.

Each possibility represents a separate embodiment of the present invention.

According to yet other embodiments, the peptide is selected from the group consisting of SEQ ID NOs: 5, 6, 10, 12, 20-23, 25, 27, 30-32, 34 and 1924.

Each possibility represents a separate embodiment of the present invention.

Analogs and derivatives of the peptides are also within the scope of the present invention; as long as they maintain the peptide function e.g. compete with binding of a TCR presented on a T cell to a target of the T cell. These include but are not limited to conservative and non-conservative substitutions of amino acids, modification of the peptide's terminal (e.g. acylation of N-terminus, amidation of C-terminus etc.), insertion and deletion of amino acids within the sequence, cyclization, modification of a peptide bond, and combination of two or more such modification. Such modification and the resultant peptide analog or derivative are within the scope of the present invention as long as they confer, or even improve the immunogenicity or activity of the peptide.

Specifically, according to some embodiments, an isolated peptide analog comprising one conservative amino acid substitution, deletion or addition to the specific peptides listed above is provided.

According to some embodiments, the peptide analog, having one amino acid substitution is selected from the group consisting of SEQ ID NOs: 3, 5, 6, 8, 10, 12, 15-17, 19, 415, 1923, 2018-2032.

Each possibility represents a separate embodiment of the present invention.

The present invention further provides peptide multimers, peptide conjugates, and fusion proteins comprising peptides, analogs and derivatives according to the invention.

According to some embodiments, a fusion protein according to the invention comprises an immunogenic protein carrier, such as an immunoglobulin molecule or a T cell.

According to some embodiments, a peptide multimer comprising a plurality of identical or different peptides defined above is provided.

According to some embodiments of the invention, there is provided a multimer of the isolated peptide.

According to some embodiments of the invention, the at least two isolated peptides are identical.

According to some embodiments of the invention, the at least two isolated peptides are different.

According to some embodiments, the at least two peptides or peptide analogs are covalently linked, directly or through a spacer or a linker.

According to some embodiments, the peptide multimer comprises a linker. According to particular embodiments, the linker comprises plurality of Lysine residues. Each possibility represents a separate embodiment of the present invention.

A peptide conjugate according to the present invention comprises any peptide, peptide analog or peptide multimer defined above, conjugated or fused (e.g., covalent bond e.g., translational fusion or non-translational fusion) to a carrier protein or moiety which improves the peptide's solubility, stability or permeability (e.g., collectively termed bioavailability) or antigenicity.

According to some embodiments of the invention, the peptide is attached to a non-proteinaceous moiety.

According to some embodiments of the invention, the non-proteinaceous moiety comprises polyethylene glycol (PEG).

The peptide of present invention may be produced by any method known in the art, including recombinant and synthetic methods. According to some embodiments a synthetic peptide, peptide multimer or peptide conjugate is provided. According to other embodiments a recombinantly produced peptide, peptide multimer, peptide fusion protein or peptide conjugate with a carrier protein is provided.

Isolated polynucleotide sequences comprising at least one sequence encoding a peptide, peptide analog, conjugate or fusion protein are also included in the scope of the present invention.

According to some embodiments of the invention, there is provided an isolated polynucleotide comprising a nucleic acid sequence encoding the peptide (and as mentioned the modification thereof e.g., multimers, fusions as long as it is a translational product).

According to some embodiments of the invention, there is provided an isolated polynucleotide comprising a nucleic acid sequence encoding the agent.

According to some embodiments of the invention, there is provided an isolated polynucleotide comprising a nucleic acid sequence encoding the antibody.

According to some embodiments, a polynucleotide sequence encoding a peptide or peptide analog is translationally linked to another polynucleotide sequence such as an RNA or DNA molecule and is recombinantly expressed within target cells.

According to specific embodiments, said polynucleotide sequence is part of a nucleic acid construct also referred to herein as a vector such as a recombinant viral or bacterial vector. Vectors comprising the above polynucleotide sequences, as well as host cells, including hybridoma cells, comprising said vectors, are also within the scope of the present invention.

In another aspect the present invention is related to a pharmaceutical composition useful for preventing, attenuating or treating a disease or disorder associated T cell expressing a TCR with a specific CDR3 sequence, such as cancer, autoimmunity or allo-immunity.

According to some embodiments of the invention, there is provided a pharmaceutical composition comprising as an active ingredient the isolated agent, peptide or antibody and a pharmaceutically acceptable carrier or diluent.

According to some embodiments, the pharmaceutical composition comprises a therapeutically effective amount of a peptide, peptide analog, peptide multimer, fusion protein or conjugate or encoding nucleic acid sequence or viral or bacterial vector comprising them; and a pharmaceutically acceptable carrier or diluent.

According to some embodiments the pharmaceutical composition is formulated as a vaccine.

According to some embodiments of the invention, the pharmaceutical composition further comprises an adjuvant or a delivery system.

According to other embodiments, the formulation does not comprise an adjuvant or delivery system.

Pharmaceutically acceptable adjuvants include, but are not limited to water in oil emulsions, lipid emulsions, and liposomes.

In some embodiments the pharmaceutical composition is formulated for intramuscular, intranasal, oral, intraperitoneal, subcutaneous, topical, intradermal and transdermal delivery. In some embodiments the pharmaceutical composition is formulated for intramuscular administration. In yet other embodiments the pharmaceutical composition is formulated for intranasal administration.

The present invention further provides methods and uses of the peptides, peptide multimers and peptide conjugates for production of specific antibodies. According to some embodiments the antibodies are polyclonal antibodies. According to other embodiments, the antibodies are monoclonal antibodies. Any method known in the art for production of monoclonal or polyclonal antibodies may be used.

According to an aspect of some embodiments of the present invention there is provided a method of obtaining an antibody of interest, the method comprising using an isolated peptide of no more than 20 amino acids comprising an amino acid sequence having a CDR3 sequence of a TCR on a T cell selected from the group consisting of SEQ ID NO: 1 and SEQ ID NOs of Table 8 for producing or selecting an antibody specifically recognizing said peptide, thereby producing the antibody of interest.

According to some embodiments of the invention, the contacting is effected via immunization.

According to some embodiments of the invention, the selecting is from an antibody display platform.

According to some embodiments of the invention, the antibody display platform is selected from the group consisting of phage display, ribosome and mRNA display and microbial cell display.

The isolated agents and peptides of the present invention may be used for treating a disease associated with a T cell expressing the respective TCR-CDR3.

According to yet another aspect, the present invention provides a method of treating or alleviating an autoimmune or allograft disease or disorder comprising administering to a patient in need thereof, effective amount of a TCR CDR3 derived peptide, peptide analog, peptide multimer or peptide conjugate as defined above.

According to some embodiments of the invention the disease is an autoimmune disease.

According to some embodiments, the autoimmune disease is selected from the group consisting of: rheumatoid arthritis, multiple sclerosis, type-1 diabetes, chronic obstructive pulmonary disease (COPD), Crohn's disease, ulcerative colitis, and psoriasis.

According to some embodiments of the invention, the disease is a graft rejection disease.

According to some embodiments of the invention, the graft rejection disease is host vs. graft disease.

According to some embodiments of the invention, the disease is cancer.

According to some embodiments of the invention, the disease is pathogenic disease.

According to some embodiments of the invention, the pathogenic disease is human immunodeficiency virus or tuberculosis infection.

According to some particular embodiments, the peptide is selected from the group consisting of SEQ ID NOs: 5, 6, 10, 12, 20-23, 25, 27, 30-32, 34 and 1924.

Each possibility represents a separate embodiment of the present invention.

According to another aspect, the present invention provides antibody against a peptide derived from TCR-CDR3 segment, or an antibody fragment thereof comprising at least an antigen-binding portion.

Each possibility represents a separate embodiment of the present invention.

Each possibility represents a separate embodiment of the present invention.

According to yet other embodiments, the antibody comprises an antigen binding domain which specifically binds a sequence selected from the group consisting of SEQ ID NOs: 3, 6, 8, 10, 12, 15-17, 19, 27 and 1923.

Each possibility represents a separate embodiment of the present invention.

According to yet other embodiments, the antibody comprises an antigen recognition domain which specifically binds a sequence selected from the group consisting of SEQ ID NOs: 2-4, 8, 15-17, 19, 24, 29 and 1923.

Each possibility represents a separate embodiment of the present invention.

According to one embodiment of the present invention, the antibody is a monoclonal antibody (mAb). According to a specific embodiment, the mAb is selected from the group consisting of: mammalian antibody, humanized antibody, human antibody, chimeric antibody, and an antibody fragment comprising at least the antigen-binding portion of an antibody. According to a specific embodiment, the antibody fragment is selected from the group consisting of: Fab, Fab′, F(ab′)₂, Fd, Fd′, Fv, dAb, isolated CDR region, single chain antibody, “diabodies”, and “linear antibodies”.

Within the scope of the present invention are also nucleic acid molecules encoding an antibody or antibody fragment or monoclonal or bispecific antibody, according to the invention, having affinity and specificity for a TCR CDR3 sequence.

An antibody or antibody fragment according to the invention may be translationally linked to another protein as part of a polynucleotide molecule such as RNA or DNA.

In another aspect the present invention provides a pharmaceutical composition comprising a therapeutically effective amount of an antibody or antibody fragment comprising at least an antigen-binding portion, which specifically binds to a peptide according to the invention; and a pharmaceutically acceptable carrier or diluent.

According to some embodiments, a pharmaceutical composition comprising an antibody defined above, useful for preventing, attenuating or treating a malignancy is provided wherein the antibody recognizes a TCR CDR3 sequence and is specific for the T cells that down-regulate tumor-associated autoimmunity.

According to some embodiment, the pharmaceutical composition comprises a therapeutically effective amount of an antibody which comprises an antigen recognition domain which specifically binds a sequence selected from the group consisting of SEQ ID NOs: 2-4, 8, 15-17, 19, 24, 29 and 1923.

Each possibility represents a separate embodiment of the present invention.

In yet another aspect the present invention is related to a method of attenuating or treating a malignancy comprising administering to a subject in need thereof a pharmaceutical composition comprising a therapeutically effective amount of an antibody which recognizes a TCR CDR3 sequence specific for the T cells that down-regulate tumor-associated autoimmunity. According to this aspect, the compositions and methods are used to up-regulate effector immunity or to deprive tumor of its ability to down-regulate T cell immune intervention.

According to some embodiments, the method comprises a combined treatment regimen of an antibody according to the invention and a peptide, analog, peptide conjugate, or fusion protein according to the invention. Such administration may be performed in a combined composition or in separate compositions administered together or at separate times.

According to some embodiments, the malignancy is a metastatic cancer.

According to other embodiments, the malignancy is a solid cancer.

According to yet another aspect, the present invention provides a method of preventing or treatment tumor metastasis comprising administering to a subject in need thereof a pharmaceutical composition comprising at least one peptide, peptide analog, peptide multimer, peptide conjugate, fusion protein, antibody, or antibody fragment disclosed above.

According to some embodiments the metastasis is decreased. According to other embodiments, the metastasis is prevented. According to yet other embodiments, the spread of tumors to the lungs of said subject is inhibited.

The pharmaceutical composition according to the present invention may be administered together with an anti-neoplastic composition. According to a specific embodiment, the anti-neoplastic composition comprises at least one chemotherapeutic agent. The chemotherapeutic agent, which could be administered separately or together with the antibody according to the present invention, may comprise any such agent known in the art exhibiting anti-cancer activity, including but not limited to: mitoxantrone, topoisomerase inhibitors, spindle poison vincas: vinblastine, vincristine, vinorelbine (taxol), paclitaxel, docetaxel; alkylating agents: mechlorethamine, chlorambucil, cyclophosphamide, melphalan, ifosfamide; methotrexate; 6-mercaptopurine; 5-fluorouracil, cytarabine, gemcitabin; podophyllotoxins: etoposide, irinotecan, topotecan, dacarbazin; antibiotics: doxorubicin (adriamycin), bleomycin, mitomycin; nitrosoureas: carmustine (BCNU), lomustine, epirubicin, idarubicin, daunorubicin; inorganic ions: cisplatin, carboplatin; interferon, asparaginase; hormones: tamoxifen, leuprolide, flutamide, and megestrol acetate. According to a specific embodiment, the chemotherapeutic agent is selected from the group consisting of alkylating agents, antimetabolites, folic acid analogs, pyrimidine analogs, purine analogs and related inhibitors, vinca alkaloids, epipodopyllotoxins, antibiotics, L-asparaginase, topoisomerase inhibitor, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. According to another embodiment, the chemotherapeutic agent is selected from the group consisting of 5-fluorouracil (5-FU), leucovorin (LV), irenotecan, oxaliplatin, capecitabine, paclitaxel and doxetaxel. Two or more chemotherapeutic agents can be used in a cocktail to be administered in combination with administration of the antibody or fragment thereof.

According to a specific embodiment, the invention provides a method of treating cancer in a subject, comprising administering to the subject effective amounts of an antibody or antibody fragment according to the invention.

In another aspect, the present invention provides a method for increasing the duration of survival of a subject having cancer, comprising administering to the subject a composition comprising effective amounts of an antibody or antibody fragment defined above, and optionally an anti-neoplastic composition whereby the administration of the antibody effectively increases the duration of survival.

In yet another aspect, the present invention provides a method for increasing the progression free survival of a subject having cancer, comprising administering to the subject a composition comprising effective amounts of an antibody, or antibody fragment defined above, and optionally an anti-neoplastic composition, whereby administration of the antibody or antibody fragment effectively increases the duration of progression free survival.

Furthermore, the present invention provides a method for treating a subject having cancer, comprising administering to the subject effective amounts of an antibody or antibody fragment defined above, and optionally anti-neoplastic composition whereby administration of the antibody or antibody fragment effectively increases the response incidence in the group of subjects.

In yet another aspect, the present invention provides a method for increasing the duration of response of a subject having cancer, comprising administering to the subject a composition comprising effective amounts of an antibody or antibody fragment defined above, and optionally an anti-neoplastic composition, wherein said anti-neoplastic composition comprises at least one chemotherapeutic agent, whereby administration of the antibody or antibody fragment effectively increases the duration of response.

In another aspect, the invention provides a method of preventing or inhibiting development of metastasis in a patient having cancer, comprising administering to the subject a composition comprising effective amounts of an antibody or antibody fragment defined above and optionally an anti-neoplastic composition, whereby administration of the antibody or antibody fragment effectively increases the duration of response.

Another aspect of the present invention relates to the use of an antibody defined above or an antibody fragment thereof, for the manufacture of a therapeutic composition for the treatment of a cancer.

According to another aspect, the present invention provides a method of preventing tumor recurrence comprising administering to a subject in need thereof an antibody or antibody fragment defined above, in conjugation with surgery, radio- or chemotherapy.

A pharmaceutical composition according to the invention, comprising an antibody or fragment thereof may be administered to a subject in need thereof, by any administration route, including but not limited to: intramuscular, intravenous, oral, intraperitoneal, subcutaneous, topical, intradermal or transdermal delivery.

According to some embodiments, the composition is administered by a route selected from the group consisting of: subcutaneous injection (SC), intra-peritoneal (IP) injection, intra-muscular (IM) injection and intra-venous (IV) injection.

According to some embodiments, the compositions and treatments comprising antibody or antibody fragment are specific for the T cells that down-regulate tumor-associated autoimmunity and can unleash the otherwise suppressed effector immunity without affecting unrelated T cell responses.

According to yet another aspect, the invention provides method for up-regulating effector immunity against pathogens, comprising administering to a patient in need thereof a pharmaceutical composition comprising an antibody, or a fragment thereof, which recognizes a TCR CDR3 peptide.

According to some embodiments, the pathogen is selected from the group consisting of: human immunodeficiency virus or tuberculosis.

The present invention provides, according to another aspect a method of selecting a TCR CDR3 peptide relevant to human immunity, comprising the steps of:
i. searching for TCR sequences that were annotated to be associated with a defined immune function;
ii. comparing the sequences obtained in (i) with a TCR CDR3 dataset obtained from a large number of mammalian individuals;
iii. selecting the sequences that are found in more than 75% of the mammalian individuals of (ii); and optionally
iv. selecting from the sequences of (iii) the sequences which are also present in human TCR CDR3 dataset.

According to some embodiments, the immune function is selected from the group consisting of autoimmunity, pathogenic immunity, tumor immunity and graft rejection. Further embodiments and the full scope of applicability of the present invention will become apparent from the detailed description given hereinafter.

### BRIEF DESCRIPTION OF THE SEVERAL VIEWS OF THE DRAWINGS

FIG. 1 depicts analysis of the TCRβ repertoire of 28 C57BL/6 mice. The number of CDR3 amino acid sequences found in each sharing category. 2.5×10⁵ sequences (˜69%) are private; 289 sequences (˜0.08%) are public.

FIG. 2 shows normalized cumulative frequencies of the sequences from FIG. 1, in each sharing category. Private CDR3 sequences account for 19±5% of all sequences in each sample; the 289 public sequences account for 10±5% of all sequences in each sample.

FIG. 3 represents convergent recombination of all CDR3 sequences, as a function of sharing level. Each black dot represents the total number of nucleotide sequences coding for the same CDR3 amino acid sequence (summed across all mice in which this sequence is found). The white dots show the mean value for each sharing category.

FIG. 4 shows the average nucleotide length of private and relatively private (shared by 1 to 3 mice) and relatively public and public (shared by 26 to 28 mice) CDR3 sequences. Error bars indicate standard errors (p<8.5e-08, comparing the mean length of CDR3 nucleotide sequences shared by n=3 and by n=26 mice).

FIG. 5 represents the number of nucleotide insertions (B, p<2.2e-16), summed over the V-D and D-J junctions, in private and public sequences. Error bars indicate standard errors.

FIG. 6 shows the sharing distributions of the annotated sequences, according to their functional category. From the left—pathogen, allograft, cancer, autoimmunity.

FIG. 7 shows the comparison of annotated clones of the different functional categories, in terms of mean frequency.

FIG. 8 shows the comparison of annotated clones of the different functional categories, in terms of total number of nt insertions in the VD and DJ junctions.

FIG. 9 shows the average correlations in V segment usage calculated between all 289 public clones in the C57BL/6 H2^{b} strain dataset and the three other MHC-restricted T cell groups. Error bars indicate SEM.

FIG. 10 shows type I diabetes incidence following treatment with antibodies against relatively public CDR3 affects autoimmune type 1 diabetes. The graphs show incidence of diabetic mice in a group of male (left) and female (right) mice. Gray: a single injection of anti-CDR3 antibodies; Black: a single injection of control antibodies. The anti-CDR3 antibody increased disease incidence in both males and females.

FIG. 11 shows the effect of anti-C9-CDR3 polyclonal antibodies on tumor growth in C57BL/6 mice injected with syngeneic mouse Lewis lung carcinoma cell line (D-122), one month following tumor injection. The graph shows the number of nodules in lungs of mice treated with anti-C9-CDR3 antibody (Tumor+2*antiC9) or with control serum (tumor+2*pi). Each mouse is represented by a circle and the mean value is presented by a vertical line. * P≦0.05 by Wilcoxon rank-sum test.

### DETAILED DESCRIPTION OF THE INVENTION

The CDR30 segment of the T-cell receptor (TCR), which recognizes antigen epitopes, is formed by random recombination of V-D-J gene segments, junctional nucleotide deletions and non-templated nucleotide insertions, which renders unlikely any sharing of CDR3 sequences among individuals. Nevertheless, reports of shared TCR sequences are accumulating. To gain a repertoire-wide view of TCR sharing, CDR3 β-chain sequences were studied using high-throughput TCR-sequencing in CD4+ splenic T cells of 28 healthy C57BL/6 mice. A few hundreds of relatively public and public sequences shared by most mice were uncovered. These highly shared sequences differed from more private ones: they are two orders of magnitude more abundant on average, feature a restricted V/J segment usage, and exhibit much higher convergent recombination—tens of different nucleotide (nt) recombinations encode the same CDR3 amino-acid (aa) sequence. Public sequences were found to be enriched for previously defined, MHC-diverse CDR3 sequences that were functionally associated with autoimmune, allograft and tumor-related reactions, but less with anti-pathogen-related CDR3 sequences. Thus, public/private CDR3 discrimination marks functionally different T-cell response categories. These results suggest an ongoing positive selection of a restricted subset of self-associated, public T-cell clones and invite reexamination of the basic mechanisms of T-cell repertoire formation.

To investigate TCR publicness in a repertoire-wide manner, high-resolution maps of TCRβ repertoires of splenic CD4+ T cells in 28 individual C57BL/6 mice, were generated based on massive parallel sequencing (TCR-seq) of T-cell mRNA (W. Ndifon et al., Proc Natl Acad Sci USA 109, 15865, 2012). The mice included 12 untreated, 7 immunized with complete Freund's adjuvant (CFA) and 9 immunized with CFA+ovalbumin (OVA). About 2.4×10⁶ TCRβ CDR3 nt sequence reads were obtained, which corresponded to about 3.5×10⁵ unique (non-redundant) TCR aa sequences. A summary of the samples is presented in Table 1 (in the “Examples” section below). The analysis is focused mainly on the as sequences of the TCRβ complementarity determining region 3 (CDR3), which is the most diverse region of the TCR molecule and is associated with antigen epitope recognition. Due to the degeneracy of the genetic code, the same functional CDR3 aa sequence could result from different nt recombinations—a phenomenon termed convergent recombination (V. Venturi, et al., 2006, 2008 ibid).

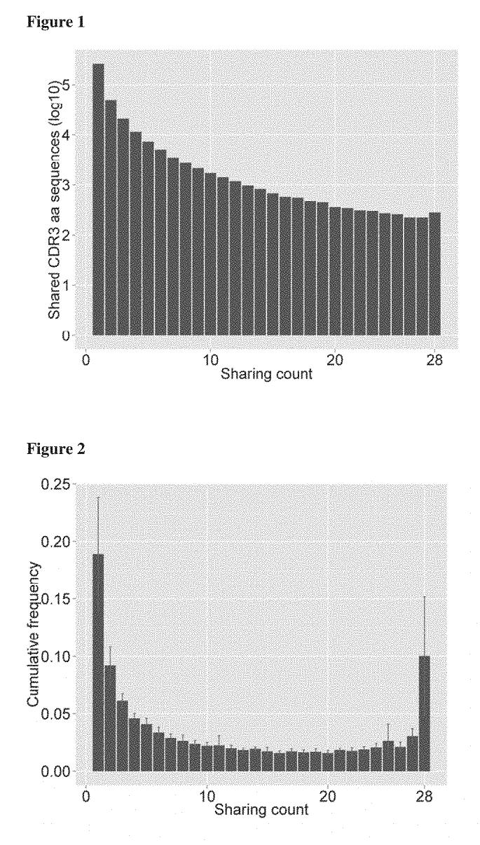

It was unexpectedly found that on average, any two mice in the dataset share 10.5±1.8% of their expressed CDR3 as sequences. There was no significant difference in pairwise sharing between the naïve and immunized groups of mice; hence, all 28 mice were combined for further analysis. Unique CDR3 as sequences were next binned according to the number of mice in which they occurred (FIG. 1). Most of the CDR3 as sequences were found in only one mouse (˜69% of all sequences). However, hundreds of sequences were highly shared among individual mice; 1,908 sequences were shared by more than 75% (n>21) of the mice (see Table 10 hereinbelow). Notably, 289 CDR3 aa sequences were found that were shared by all 28 mice (˜0.08% of all sequences) (see Table 10 hereinbelow). Thus, TCRβ CDR3 as sequences have a wide range of sharing levels, from private to highly public. A sequence was defined as private if it appeared in only one mouse in the dataset used in the present invention, as relatively private if it was shared by 2-7 mice, as relatively public if shared by 22-27 mice, and as public if shared by all 28 mice.

The frequency of each CDR3 as sequence was next analyzed as a function of its degree of sharing. CDR3 sequence frequency reflects two factors: the number of T cells bearing that aa sequence (herein termed the CDR3-type) and the amount of relevant mRNA produced by a cell. Thus, the frequency of a sequence reflects the numbers and the activity state of the T cells that express the specific receptor sequence. A gradual increase in median frequency as a function of sharing was observed; CDR3 sequences with higher levels of sharing tended to be more abundant. Interestingly, very prominent CDR3 as sequences (relative frequency>5×10⁻⁴) appear both among private or relatively private sequences as well as among more public sequences; the frequency curve seems to dip for intermediate levels of sharing, suggesting the distinctness of the most highly public subset of sequences. Since increased frequencies of TCR sequences probably result from antigen-specific T-cell clonal expansion, it is likely that the most public CDR3-types, as well as a fraction of the private CDR3-types, reflect T-cell expansion following antigen-activation.

FIG. 2 shows the cumulative frequencies of the CDR3 as sequences in each sharing category, averaged over all mice. The public subgroup of 289 CDR3 sequences constitutes 10±5% of the total sequences. Assuming similar levels of mRNA encoding TCRβ among cells of the different sharing categories, these results suggest that public CDR3 sequences represent highly abundant T cell CDR3-types; on average, the number of T cells that bear each of the public CDR3 as sequences is 50-100 times higher than that of an average private T cell CDR3-type. To confirm the abundance of public sequences their occurrence was sought in a set of 79 TCRs that was randomly cloned and sequenced using Sanger sequencing. Twenty-two of these 79 sequences (28%) were identical to one of the 289 public CDR3 as sequences derived by TCR-seq, validating by an independent method the relative abundance of public CDR3 sequences.

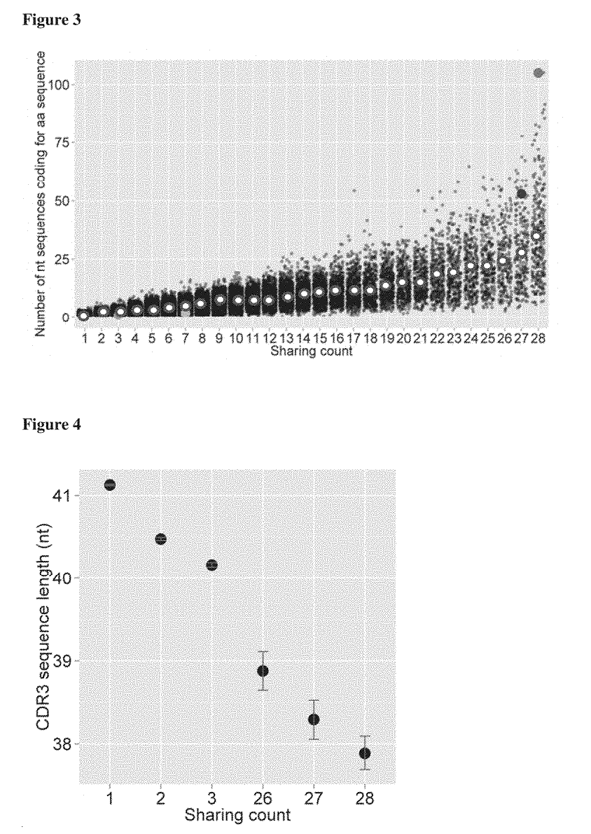

Previous studies reported that public TCRs manifest a higher level of convergent recombination (H. Li et al., J Immunol 189, 2404, 2012; M. F. Quigley et al., Proc Natl Acad Sci USA 107, 19414, 2010; V. Venturi et al., J Immunol 186, 4285, 2011). The analysis demonstrated in the present invention, of a large number of individuals revealed a continuous trend; increased sharing was associated with a gradual increase in the degree of convergent recombination (FIG. 3); private CDR3 aa sequences are encoded on average by one nt sequence, the public sequences are encoded by 34.5 nt sequences on average.

The pattern of convergent recombination of nt sequences for 4 CDR3 as sequences were studied. The two more public sequences, found in 28 and 27 of the mice, show high convergent recombination (encoded by 105 and 53 nt sequences, respectively). There is no dominating nt sequence in any mouse, nor a dominant nt sequence across mice. In contrast, two relatively private CDR3 as sequences, present in 7 and 3 mice, manifest a limited number of nt sequences. Thus, private and public CDR3 segments differ markedly in their detectable degree of convergent recombination.

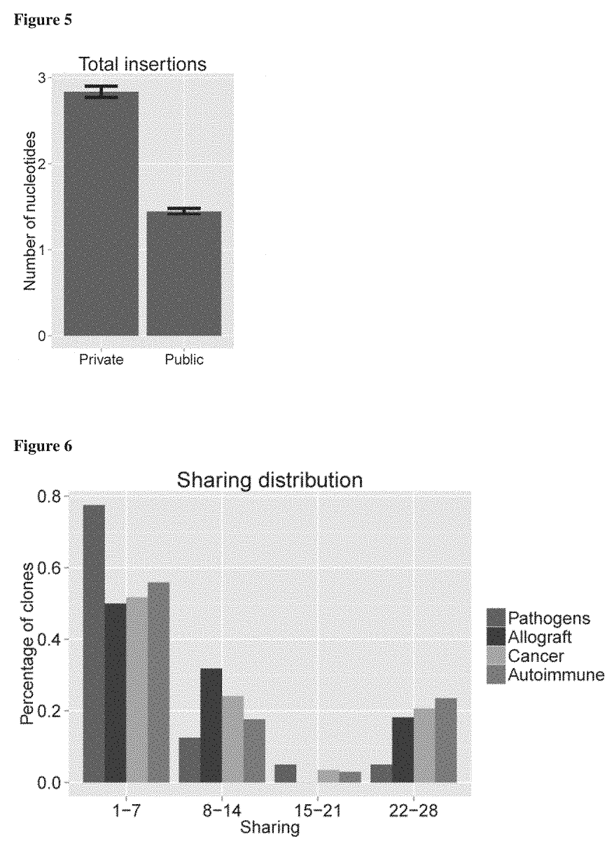

Further analysis of the public CDR3 sequences revealed other distinct characteristics. FIG. 4 shows the mean CDR3 lengths for private and relatively private sequences (shared by 1 to 3 mice) and public and relatively public ones (shared by 26 to 28 mice). The more public CDR3 as sequences tend to be shorter on average by about one aa residue and, in addition, show a significantly lower number of nt insertions in the VD and DJ junctions (FIG. 5); this suggests that public sequences are closer to germline configurations. However, the number of junctional nt deletions in both public and private sequences is similar. Public CDR3 sequences also manifest a skewed and restricted V and J segment usage compared with private sequences; three V genes (V2, V15 and V18) are not used, and other V and J genes are represented at significantly different frequencies. The skewed and restricted V and J frequencies are also compatible with antigen-specific selection of these public T cell CDR3-types.

The marked differences between public and private CDR3 sequences suggest that each class might be driven by different classes of antigens. Interestingly, a sequence (C9: CASSLGGNQDTQYF, SEQ ID NO: 1), which was previously found to be public in NOD mice that spontaneously develop autoimmune type 1 diabetes (Y. Tikochinski et al., Int Immunol 11, 951, 1999), is relatively public in the dataset of healthy C57BL/6 mice (shared by 27 mice). The C9 CDR3 sequence was found to recognize a peptide epitope (p277) in the mouse/human HSP60 molecule; administration of peptide p277 to NOD mice activates anti-C9 and other regulatory T cells (D. Elias, et al., Int Immunol 11, 957, 1999), and arrests the destruction of pancreatic beta cells both in NOD mice (D. Elias, I. R. Cohen, Lancet 343, 704, 1994) and in humans with recent-onset type 1 diabetes (I. Raz et al., Lancet 358, 1749, 2001). Despite the fact that the NOD and the C57BL/6 mouse strains differ in their MHC haplotypes (H2^{g7} and H2^{b}, respectively), it was now found that the same CDR30 as sequence is public in both.

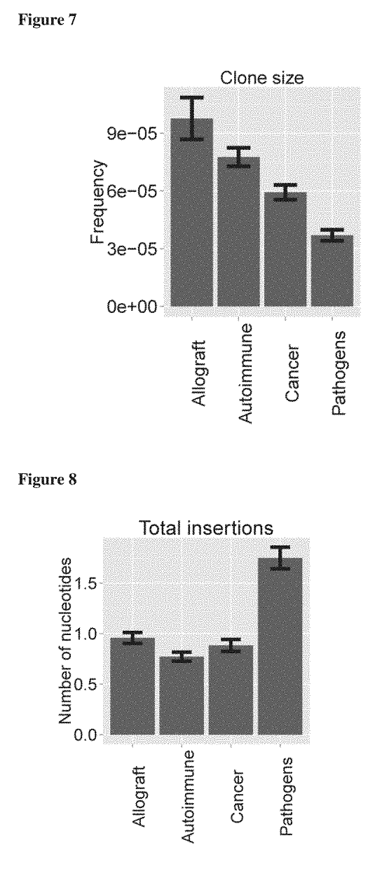

The literature was then searched for additional annotated TCRβ sequences in various models in different strains of mice bearing varying MHC haplotypes. 252 TCRβ sequences that were previously annotated to be associated with defined immune functions were collected from the literature, and compared with the CDR3 dataset. The annotated sequences were associated with four categories of immune reactions: a) Immunity to foreign pathogens; b) Allograft reactions; c) Tumor-related T cells; and d) Autoimmune conditions. Of the 252 annotated CDR3 sequences, 124 sequences were identified that were also present in one or more of our 28 healthy C57BL/6 mice (see Table 11 hereinbelow). The 124 annotated sequences associated functionally with autoimmunity, allograft rejection and cancer (self or modified self) were relatively enriched with shared, relatively public and public sequences compared with the sequences associated with anti-virus or anti-malarial immunity. This is evident from FIG. 6, which shows the sharing distribution of sequences of the 4 functional categories (From the left—pathogen, allograft, cancer, autoimmunity). It was found that ˜20% of the annotated sequences associated with categories of self or modified self-antigens are relatively public or public (found in >21 mice). In contrast, only ˜5% of the sequences associated with reactivity to non-self-associated antigens showed similar publicness. Moreover, the CDR3 sequences associated with autoimmune, cancer and allograft annotated immunity showed characteristic similarities that were identified in the more public sequences: a higher frequency (FIG. 7, p=1.16e-12, p=3.9e-06 and p=1.6e-07 respectively); a higher degree of convergent recombination (p<2.2e-16, p=4.1e-08 and p=4.5e-09 respectively); and fewer nt insertions (FIG. 8, p=4.3e-10, p=1e-08 and p=6.3e-08 respectively), compared with the anti-pathogen-related sequences.

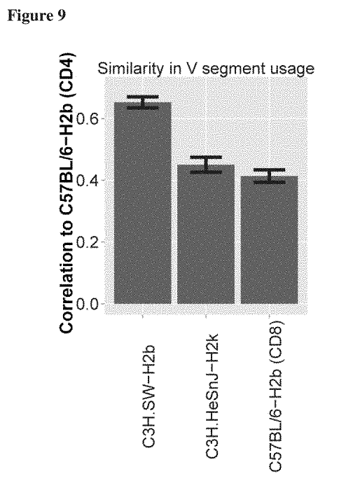

As noted above, the annotated sequences were derived from various mouse strains that differed in their MHC haplotypes. To further explore the MHC restrictions of the public sequences, TCR-sequencing was used to map the repertoires of T cells interacting with different MHC molecules: C57BL/6 CD8+ T cells (which are restricted by MHC class-I H2^{b}); C3H.SW CD4+ T cells (which have the H2^{b} MHC allele, but different genetic background than C57BL/6); and C3H.HeSnJ CD4+ T cells (which are congenic with the C3H.SW strain but bear the H2^{k} allele). These repertories were compared with those of the MHC-II H2^{b} restricted CD4+ T cells of the C57BL/6 mice. It was found that >82% of the 289 public CDR3 as sequences were also present in the other T cell repertories. Interestingly, most of these public CDR3 sequences were associated with several different TCRβ V region gene segments. Moreover, the V gene segments associated with each shared CDR3 as sequence tended to differ between the different MHC-restricted T cell groups. A global analysis of the degree of similarity in V-segment usage of the public CDR3 sequences between C57BL/6 (H2^{b}) CD4+ T cells and the other T cell groups shows that differences in MHC restriction are associated with more diverse V gene usage (FIG. 9). For example, the C3H.SW strain bearing the H2^{b} haplotype shows a pattern of V segment usage that is more similar to the C57BL/6 (also H2^{b} haplotype) than is the C3H.HeSnJ strain that carries the H2^{k} haplotype. This data imply that V segment usage by public T cells is dominated by MHC haplotype to a greater extent than by the non-MHC genetic background. This observation is compatible with structural studies, which show that the CDR1 and CDR2 segments of the TCR, which are expressed on the V-gene segments of the TCR outside the CDR3 region, interact directly with the MHC molecule (M. G. Rudolph, R. L. Stanfield, L A. Wilson, Annu Rev Immunol 24, 419, 2006; E. S. Huseby et al., Cell 122, 247, 2005). Shared CDR3 segments with variable V regions is not limited to the TCR; CDR3 regions of the immunoglobulin heavy chain associated with different V segments have also been reported in the B-cell receptor (BCR) antibody responses of humans to dengue virus (P. Parameswaran et al., Cell Host Microbe 13, 691, 2013).

Without wishing to be bound to any theory or mechanism of action, it is suggested that the high level of convergent recombination of public sequences together with their greater abundance relative to the more private sequences could result mainly from two mechanisms: a) biases in the recombination process that favor the generation of certain sequences, which renders them more abundant and more public, and b) different degrees of positive selection by particular antigen epitopes, such that the more public CDR3 aa types would enjoy a selective advantage, particularly in the process of tonic stimulation needed to preserve TCR repertoires in the periphery (K. Hochweller et al., Proc Natl Acad Sci USA 107, 5931, 2010), which is where they were sampled. The two mechanisms can function together: recombination biases ensure the initial presence of certain public clones in different individuals, and selection that leads to clonal expansion differentially prevents their subsequent loss. The restricted pattern of V/J segment usage by public clones, the high level of convergent recombination and the finding of the same CDR3 as sequences among T cells interacting with different types of MHC molecules, are all in favor of positive selection as a dominant mechanism in the generation of public CDR3-types. According to this hypothesis, public CDR3-types are those stimulated to proliferate by frequent contact with high amounts of their cognate antigens. In contrast, private CDR3-types would be those that meet their cognate antigens only rarely or sporadically, and so would proliferate less often without accumulation of convergent recombined sequences over time. In other words, private and public CDR3-types might express the degree and dynamics of their contact with cognate antigens subsequent to genetic recombination in the thymus.

The finding of annotated CDR3 sequences (see Table 11 hereinbelow) in the dataset of healthy mice, presented herein for the first time, highlights a functional difference between the more private TCR sequences, which was found to be associated with all classes of antigens, and the more public sequences, which appear to be associated mainly with autoimmune conditions, allograft reactions and tumor infiltration (FIGS. 6-9). The standard clonal selection paradigm of adaptive immunity would predict that T cells expressing TCRs capable of binding to self-antigens must be deleted during development, most likely in the thymus. Yet, as shown here, a set of autoimmune CDR3 as sequences are commonly shared and even appear to be amplified with high frequency and convergent recombination. This finding is compatible with the hypothesis that the high convergent recombination and frequency of public CDR3-types are likely to reflect frequent encounter with cognate antigens; indeed, self-like antigens are continuously present while foreign viral antigens are encountered only as a result of sporadic infection or immunization.

The high frequency of public CDR3 TCR as sequences associated with autoimmunity is used, according to the present invention, as a source of therapeutic peptides against autoimmune disorders and graft rejection and neutralizing antibodies for cancer therapy; Modulating public CDR3-types identified herein might provide a new therapeutic approach to modulating autoimmune disease. TCR diversity has been an obstacle for treatments such as T-cell vaccination based on specific TCR sequences (I. R. Cohen, Vaccine 20, 706, 2001), which might be alleviated if public TCRs can be used as effective T-cell vaccines.

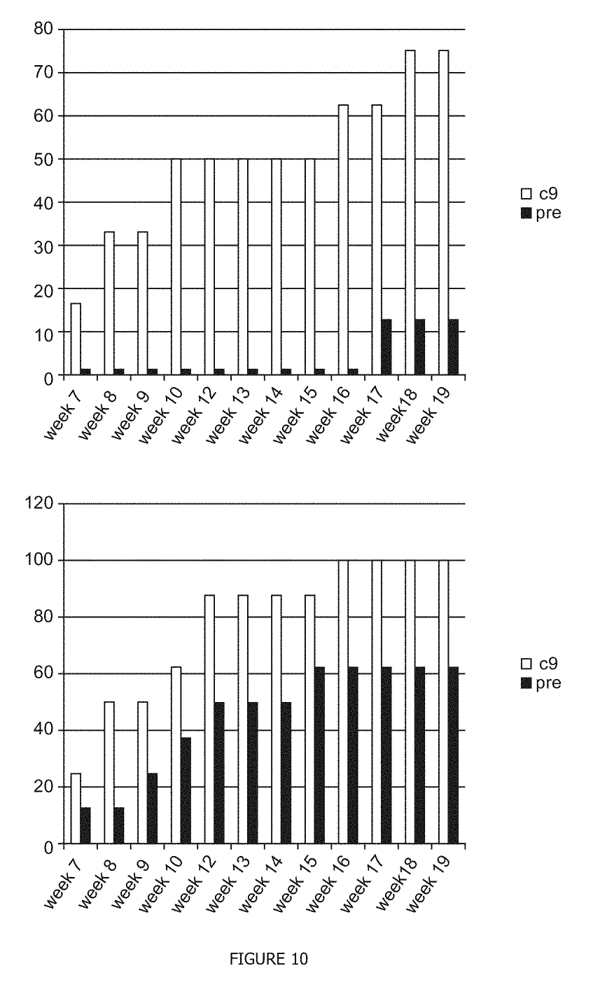

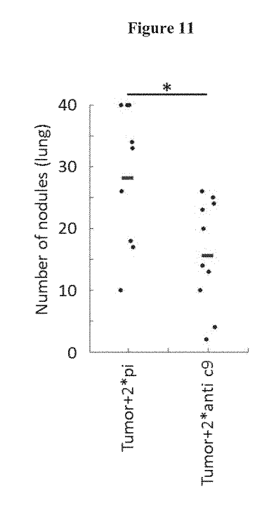

Based on the present teachings, the inventors were able to show that an antibody raised against the C9 relatively public peptide was able to unleash an autoimmune response in NOD mice, a model for type 1 Diabetes (Example 2 and FIG. 10), and on the other hand to inhibit tumor growth in a syngeneic lung carcinoma mouse tumor model (Example 3 and FIG. 11), substantiating the robustness of targeting the CDR3 in disease treatment.

Currently, there are no specific drugs or clinically-used antibodies that target CDR3 peptides for cancer immunotherapy. This is mainly due to 2 reasons: a) The enormous size of the TCR repertoire, which precluded effective identification of potential targets; b) The fact that most CDR3 sequences are private or exist only in a small number of individuals, thus making them highly individualized targets that cannot be hit by simple reagents.

The discoveries represented herein for the first time open a possibility to overcome both limitations. First, using high-throughput methodologies, millions of CDR3 sequences were scanned. Using these new methodologies, CDR3 sequences that are highly shared among mice (also across MHC barriers) were identified; some of these are shared also in humans, and are related to sequences annotated in various cancer models. Thus, a set of specific candidate sequences that can serve as potential targets was identified. Second, the high level of sharing of these CDR3 sequences among individuals can provide highly specific targets that are still found in a large fraction of patients, indicating the universality of these novel therapeutic agents.

Thus, the present inventors have identified CDR3 sequences which are shared between mice strains and even by human and mice which may be used per se or as targets for immunotherapy using dedicated agents.

Thus, according to a specific embodiment, there is provided an isolated agent capable of at least one of:
(i) binding a TCR presented on a T cell;
(ii) competing with binding of a TCR presented on a T cell to a target of said T cell;
(iii) eliciting a specific immune-response of a T cell; and
(iv) eliciting a specific immune-response against a T cell;
wherein said T cell is expressing a TCR-CDR3 sequence comprising an amino acid sequence selected from the group consisting of SEQ ID NOs of Table 8, wherein when said agent is a peptide it is selected from the group consisting of SEQ ID NOs of Table 7.

According to a specific embodiment, the CDR3 sequence is selected from the group consisting of the sequences in Table 2.

According to a specific embodiment, the CDR3 sequence is selected from the group consisting of the sequences in Table 9.

According to a specific embodiment, the CDR3 sequence is selected from the group consisting of the sequences in Table 3.

According to another specific embodiment, the CDR3 sequence is selected from the group consisting of the sequences in Table 4.

According to another specific embodiment, the CDR3 sequence is selected from the group consisting of the sequences in Table 5.

According to yet another specific embodiment, the CDR3 sequence is selected from the group consisting of the sequences in Table 6.

According to another specific embodiment, the CDR3 sequence is selected from the group consisting of the sequences in Table 7.

Additional CDR3 sequences which can be used according to the present teachings can be identified according to a method comprising the steps of:
i. searching for TCR sequences that were annotated to be associated with a defined immune function;
ii. comparing the sequences obtained in (i) with a TCR CDR3 dataset obtained from a large number of mammalian individuals;
iii. selecting the sequences that are found in more than 75% of the mammalian individuals of (ii); and optionally
iv. selecting from the sequences of (iii) the sequences which are also present in human TCR CDR3 dataset.

According to specific embodiments, the immune function is selected from the group consisting of: autoimmunity, pathogenic immunity, tumor immunity and graft rejection.

According to a specific embodiment, the CDR3 of the invention are selected from the group of private, public, relatively private and relatively public.

As used herein the term “private” refers to a CDR3 sequence present in 1 of the mammalian individuals tested in a dataset.

As used herein, the term “relatively private” refers to a CDR3 sequence present in at least 2 and not more than 25% of the mammalian individuals tested in the dataset.

As used herein, the term “relatively public” refers to a CDR3 sequence present in 75%-98% of the mammalian individuals tested in the dataset.

As used herein, the term “public” refers to a CDR3 sequence present in 98%-100% of the mammalian individuals tested in the dataset.

As used herein the term “T cell” refers to a differentiated lymphocyte with a CD3⁺, TCR⁺ having either CD4⁺ or CD8⁺ phenotype. The T cell may be either an effector or a regulatory T cells.

As used herein, the term “effector T cells” refers to a T cell that activates and direct other immune cells e.g. by producing cytokines or has a cytotoxic activity e.g., CD4+, Th1/Th2, CD8+ cytotoxic T lymphocyte.

As used herein, the term “regulatory T cell” or “Treg” refers to a T cell that negatively regulates the activation of other T cells, including effector T cells, as well as innate immune system cells. Treg cells are characterized by sustained suppression of effector T cell responses. According to a specific embodiment, the Treg is a CD4+CD25+Foxp3+ T cell.

As used herein the term “T cell receptor” or “TCR” refers to an antigen-recognition molecule present on the surface of T cells and may comprise the TCRα chain, the TCRβ chain, the TCRγ chain or the TCRδ chain.

According to a specific embodiment, TCR refers to the TCRβ chain.

As used herein, the terms “complementarity-determining region” or “CDR” are used interchangeably to refer to the hypervariable regions found within the variable region of an antibody or a TCR chain. Generally, each of a heavy chain of an antibody, a light chain of an antibody, a TCRα chain and TCRβ chain comprise three CDRs, CDR1, CDR2 and CDR3. Typically, CDR3 in TCR is the main CDR responsible for recognizing processed antigen.

The identity of the amino acid residues in a particular TCR that make up a variable region or a CDR can be determined using methods well known in the art and include methods such as sequence variability as defined by Kabat et al. (See, e.g., Kabat et al., 1992, Sequences of Proteins of Immunological Interest, 5th ed., Public Health Service, NIH, Washington D.C.), location of the structural loop regions as defined by Chothia et al. (see, e.g., Chothia et al., Nature 342:877-883, 1989.), a compromise between Kabat and Chothia using Oxford Molecular's AbM antibody modeling software (now Accelrys®, see, Martin et al., 1989, Proc. Natl Acad Sci USA. 86:9268; and world wide web site www.bioinf-org.uk/abs), available complex crystal structures as defined by the contact definition (see MacCallum et al., J. Mol. Biol. 262:732-745, 1996), the “conformational definition” (see, e.g., Makabe et al., Journal of Biological Chemistry, 283:1156-1166, 2008) and IMGT [Lefranc M P, et al. (2003) IMGT unique numbering for immunoglobulin and T cell receptor variable domains and Ig superfamily V-like domains. Dev Comp Immunol 27: 55-77]. According to a specific embodiment, the CDR3 region is defined as starting from the last conserved cysteine of Vβ and ending at the first position of the conserved amino acid motif [F|H][A|G]XG of Jβ, where X denotes any amino acid.

As used herein the term “agent” refers to a substance capable of at least one of:
(i) binding a TCR presented on a T cell; For example, an antibody which comprises an antigen binding domain which specifically binds the TCR-CDR3 (e.g., activating or neutralizing antibodies as described hereinbelow).
(ii) competing with binding of a TCR presented on a T cell to a target of the T cell; For example, a peptide having a CDR3 amino acid sequence sufficient to compete with the binding of the TCR clone to its target cell.
(iii) eliciting a specific immune-response of a T cell; For example, a peptide to which the CDR3 may bind or an activating antibody as further described hereinbelow; and
(iv) eliciting a specific immune-response against a T cell; The immunizing agent can be a peptide, an antibody or a polynucleotide for example such as a DNA vaccine. More specifically, a peptide as described herein may elicit an antibody response when administered to the subject. This antibody response will be directed to the T cell. Alternatively or additionally, antibody administration may elicit complement activity to the T cells bound thereby

The agent may be capable of at least one or two of the above-provided properties i.e.: (i); (ii); (iii); (iv), (i)+(iii); (ii)+(iii); (i)+(iv) and (ii)+(iv).

Thus, on-limiting examples of an agent include antibody, T cell, peptide and polynucleotide.

According to specific embodiments the agent is a peptide.

Thus, according to an aspect of the present invention there is provided an isolated peptide of no more than 20 amino acids comprising an amino acid sequence having a CDR3 sequence of a TCR on a T cell, said CDR3 sequence being selected from the group consisting of SEQ ID NOs of Table 7.

According to a specific embodiment, the peptide amino acid sequence is selected from the group consisting of SEQ ID NOs of Table 2.

According to a specific embodiment, the peptide amino acid sequence is selected from the group consisting of SEQ ID NOs of Table 9.

According to a specific embodiment, the peptide amino acid sequence is selected from the group consisting of SEQ ID NOs of Table 3.

According to a specific embodiment the peptide amino acid sequence is selected from the group consisting of SEQ ID NOs of Table 4.

According to a specific embodiment, the peptide amino acid sequence is selected from the group consisting of SEQ ID NOs of Table 5.

According to a specific embodiment, the peptide amino acid sequence is selected from the group consisting of SEQ ID NOs of Table 6.

According to a specific embodiment, the peptide amino acid sequence is selected from the group consisting of SEQ ID NOs of Table 7.

According to yet other embodiments, the peptide is selected from the group consisting of SEQ ID NOs: 5, 6, 10, 12, 20-23, 25, 27, 30-32, 34 and 1924.

According to an embodiment, the peptides are selected non-immunogenic in a subject.

The term “peptide” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder. Although peptide mimetics, analogs and derivatives are contemplated, it is still very important to maintain the function of the peptides either in vivo (ex-vivo) or in-vitro such as for generating antibodies to TCR-CDR3.

Peptide bonds (—CO—NH—) within the peptide may be substituted, for example, by N-methylated amide bonds (—N(CH3)-CO—), ester bonds (—C(═O)—O—), ketomethylene bonds (—CO—CH2-), sulfinmylmethylene bonds (—S(═O)—CH2-), α-aza bonds (—NH—N(R)—CO—), wherein R is any alkyl (e.g., methyl), amine bonds (—CH2-NH—), sulfide bonds (—CH2-S—), ethylene bonds (—CH2-CH2-), hydroxyethylene bonds (—CH(OH)—CH2-), thioamide bonds (—CS—NH—), olefinic double bonds (—CH═CH—), fluorinated olefinic double bonds (—CF═CH—), retro amide bonds (—NH—CO—), peptide derivatives (—N(R)—CH2-CO—), wherein R is the “normal” side chain, naturally present on the carbon atom.

These modifications can occur at any of the bonds along the peptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The peptides of some embodiments of the invention may also include one or more modified amino acids or one or more non-amino acid monomers (e.g. fatty acids, complex carbohydrates etc).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 12 and 13 below list naturally occurring amino acids (Table 12), and non-conventional or modified amino acids (e.g., synthetic, Table 13) which can be used with some embodiments of the invention.

TABLE 12

| Amino Acid | Three-Letter Abbreviation | One-letter Symbol |
|---|---|---|
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Set | S |
| Threonine | Thr | T |
| Tryptophan | Trp | W |
| Tyrosine | Tyr | Y |
| Valine | Val | V |
| Any amino acid as above | Xaa | X |

TABLE 13

| Non-conventional amino acid | Code | Non-conventional amino acid | Code |
|---|---|---|---|
| ornithine | Orn | hydroxyproline | Hyp |
| α-aminobutyric acid | Abu | aminonorbornyl- | Norb |
| | | carboxylate | |
| D-alanine | Dala | aminocyclopropane- | Cpro |
| | | carboxylate | |
| D-arginine | Darg | N-(3-guanidinopropyl)glycine | Narg |
| D-asparagine | Dasn | N-(carbamylmethyl)glycine | Nasn |
| D-aspartic acid | Dasp | N-(carboxymethyl)glycine | Nasp |
| D-cysteine | Dcys | N-(thiomethyl)glycine | Ncys |
| D-glutamine | Dgln | N-(2-carbamylethyl)glycine | Ngln |
| D-glutamic acid | Dglu | N-(2-carboxyethyl)glycine | Nglu |
| D-histidine | Dhis | N-(imidazolylethyl)gycine | Nhis |
| D-isoleucine | Dile | N-(1-methylpropyl)glycine | Nile |
| D-leucine | Dleu | N-(2-methylpropyl)glycine | Nleu |
| D-lysine | Dlys | N-(4-aminobutyl)glycine | Nlys |
| D-methionine | Dmet | N-(2-methylthioethyl)glycine | Nmet |
| D-ornithine | Dorn | N-(3-aminopropyl)glycine | Norn |
| D-phenylalanine | Dphe | N-benzylglycine | Nphe |
| D-proline | Dpro | N-(hydroxymethyl)glycine | Nser |
| D-serine | Dser | N-(1-hydroxyethyl)glycine | Nthr |
| D-threonine | Dthr | N-(3-indolylethyl) glycine | Nhtrp |
| D-tryptophan | Dtrp | N-(p-hydroxyphenyl)glycine | Ntyr |
| D-tyrosine | Dtyr | N-(1-methylethyl)glycine | Nval |
| D-valine | Dval | N-methylglycine | Nmgly |
| D-N-methylalanine | Dnmala | L-N-methylalanine | Nmala |
| D-N-methylarginine | Dnmarg | L-N-methylarginine | Nmarg |
| D-N-methylasparagine | Dnmasn | L-N-methylasparagine | Nmasn |
| D-N-methylasparatate | Dnmasp | L-N-methylaspartic acid | Nmasp |
| D-N-methylcysteine | Dnmcys | L-N-methylcysteine | Nmcys |
| D-N-methylglutamine | Dnmgln | L-N-methylglutamine | Nmgln |
| D-N-methylglutamate | Dnmglu | L-N-methylglutamic acid | Nmglu |
| D-N-methylhistidine | Dnmhis | L-N-methylhistidine | Nmhis |
| D-N-methylisoleucine | Dnmile | L-N-methylisolleucine | Nmile |
| D-N-methylleucine | Dnmleu | L-N-methylleucine | Nmleu |
| D-N-methyllysine | Dnmlys | L-N-methyllysine | Nmlys |
| D-N-methylmethionine | Dnmmet | L-N-methylmethionine | Nmmet |
| D-N-methylornithine | Dnmorn | L-N-methylornithine | Nmorn |
| D-N-methylphenylalanine | Dnmphe | L-N-methylphenylalanine | Nmphe |
| D-N-methylproline | Dnmpro | L-N-methylproline | Nmpro |
| D-N-methylserine | Dnmser | L-N-methylserine | Nmser |
| D-N-methylthreonine | Dnmthr | L-N-methylthreonine | Nmthr |
| D-N-methyltryptophan | Dnmtrp | L-N-methyltryptophan | Nmtrp |
| D-N-methyltyrosine | Dnmtyr | L-N-methyltyrosine | Nmtyr |
| D-N-methylvaline | Dnmval | L-N-methylvaline | Nmval |
| L-norleucine | Nle | L-N-methylnorleucine | Nmnle |
| L-norvaline | Nva | L-N-methylnorvaline | Nmnva |
| L-ethylglycine | Etg | L-N-methyl-ethylglycine | Nmetg |
| L-t-butylglycine | Tbug | L-N-methyl-t-butylglycine | Nmtbug |
| L-homophenylalanine | Hphe | L-N-methyl-homophenylalanine | Nmhphe |
| α-naphthylalanine | Anap | N-methyl-α-naphthylalanine | Nmanap |
| penicillamine | Pen | N-methylpenicillamine | Nmpen |
| γ-aminobutyric acid | Gabu | N-methyl-γ-aminobutyrate | Nmgabu |
| cyclohexylalanine | Chexa | N-methyl-cyclohexylalanine | Nmchexa |
| cyclopentylalanine | Cpen | N-methyl-cyclopentylalanine | Nmcpen |
| α-amino-α-methylbutyrate | Aabu | N-methyl-α-amino-α- | Nmaabu |
| | | methylbutyrate | |
| α-aminoisobutyric acid | Aib | N-methyl-α-aminoisobutyrate | Nmaib |
| D-α-methylarginine | Dmarg | L-α-methylarginine | Marg |
| D-α-methylasparagine | Dmasn | L-α-methylasparagine | Masn |
| D-α-methylaspartate | Dmasp | L-α-methylaspartate | Masp |
| D-α-methylcysteine | Dmcys | L-α-methylcysteine | Mcys |
| D-α-methylglutamine | Dmgln | L-α-methylglutamine | Mgln |
| D-α-methyl glutamic acid | Dmglu | L-α-methylglutamate | Mglu |
| D-α-methylhistidine | Dmhis | L-α-methylhistidine | Mhis |
| D-α-methylisoleucine | Dmile | L-α-methylisoleucine | Mile |
| D-α-methylleucine | Dmleu | L-α-methylleucine | Mleu |
| D-α-methyllysine | Dmlys | L-α-methyllysine | Mlys |
| D-α-methylmethionine | Dmmet | L-α-methylmethionine | Mmet |
| D-α-methylornithine | Dmorn | L-α-methylornithine | Morn |
| D-α-methylphenylalanine | Dmphe | L-α-methylphenylalanine | Mphe |
| D-α-methylproline | Dmpro | L-α-methylproline | Mpro |
| D-α-methylserine | Dmser | L-α-methylserine | Mser |
| D-α-methylthreonine | Dmthr | L-α-methylthreonine | Mthr |
| D-α-methyltryptophan | Dmtrp | L-α-methyltryptophan | Mtrp |
| D-α-methyltyrosine | Dmtyr | L-α-methyltyrosine | Mtyr |
| D-α-methylvaline | Dmval | L-α-methylvaline | Mval |
| N-cyclobutylglycine | Ncbut | L-α-methylnorvaline | Mnva |
| N-cycloheptylglycine | Nchep | L-α-methylethylglycine | Metg |
| N-cyclohexylglycine | Nchex | L-α-methyl-t-butylglycine | Mtbug |
| N-cyclodecylglycine | Ncdec | L-α-methyl-homophenylalanine | Mhphe |
| N-cyclododecylglycine | Ncdod | α-methyl-α-naphthylalanine | Manap |
| N-cyclooctylglycine | Ncoct | α-methylpenicillamine | Mpen |
| N-cyclopropylglycine | Ncpro | α-methyl-γ-aminobutyrate | Mgabu |
| N-cycloundecylglycine | Ncund | α-methyl-cyclohexylalanine | Mchexa |
| N-(2-aminoethyl)glycine | Naeg | α-methyl-cyclopentylalanine | Mcpen |
| N-(2,2-diphenylethyl)glycine | Nbhm | N-(N-(2,2-diphenylethyl) | Nnbhm |
| | | carbamylmethyl-glycine | |
| N-(3,3-diphenylpropyl)glycine | Nbhe | N-(N-(3,3-diphenylpropyl) | Nnbhe |
| | | carbamylmethyl-glycine | |
| 1-carboxy-1-(2,2-diphenyl | Nmbc | 1,2,3,4-tetrahydroisoquinoline-3- | Tic |
| ethylamino)cyclopropane | | carboxylic acid | |
| phosphoserine | pSer | phosphothreonine | PThr |
| phosphotyrosine | pTyr | O-methyl-tyrosine | |
| 2-aminoadipic acid | | hydroxylysine | |

The amino acids of the peptides of the present invention may be substituted either conservatively or non-conservatively.

The term “conservative substitution” as used herein, refers to the replacement of an amino acid present in the native sequence in the peptide with a naturally or non-naturally occurring amino or a peptidomimetics having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid).

As naturally occurring amino acids are typically grouped according to their properties, conservative substitutions by naturally occurring amino acids can be easily determined bearing in mind the fact that in accordance with the invention replacement of charged amino acids by sterically similar non-charged amino acids are considered as conservative substitutions.

For producing conservative substitutions by non-naturally occurring amino acids it is also possible to use amino acid analogs (synthetic amino acids) well known in the art. A peptidomimetic of the naturally occurring amino acid is well documented in the literature known to the skilled practitioner.

When affecting conservative substitutions the substituting amino acid should have the same or a similar functional group in the side chain as the original amino acid.

The following six groups each contain amino acids that are conservative substitutions for one another:

### 1) Alanine (A), Serine (S), Threonine (T);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V); and

### 6) Phenylalanine (F), Tyrosine (Y), Tryptophan (W).

“Derivatives” of the peptides of the invention as used herein covers derivatives which may be prepared from the functional groups which occur as side chains on the residues or the N- or C-terminal groups, by means known in the art, and are included in the invention as long as they remain pharmaceutically acceptable, i.e., they do not destroy the activity of the peptide, do not confer toxic properties on compositions containing it and do not adversely affect the antigenic properties thereof.

These derivatives may, for example, include aliphatic esters of the carboxyl groups, amides of the carboxyl groups produced by reaction with ammonia or with primary or secondary amines, N-acyl derivatives of free amino groups of the amino acid residues formed by reaction with acyl moieties (e.g., alkanoyl or carbocyclic aroyl groups) or O-acyl derivatives of free hydroxyl group (for example that of seryl or threonyl residues) formed by reaction with acyl moieties.

The term “analog” further indicates a molecule which has the amino acid sequence according to the invention except for one or more amino acid changes. Analogs according to the present invention may comprise also peptidomimetics. “Peptidomimetic” means that a peptide according to the invention is modified in such a way that it includes at least one non-coded residue or non-peptidic bond. Such modifications include, e.g., alkylation and more specific methylation of one or more residues, insertion of or replacement of natural amino acid by non-natural amino acids, replacement of an amide bond with other covalent bond. A peptidomimetic according to the present invention may optionally comprises at least one bond which is an amide-replacement bond such as urea bond, carbamate bond, sulfonamide bond, hydrazine bond, or any other covalent bond. The design of appropriate “analogs” may be computer assisted.

According to a specific embodiment, the peptide analogs are as said forth in SEQ ID NOs: 3, 5, 6, 8, 10, 12, 15-17, 19, 415, 1923 and 2018-2032.

“Salts” of the peptides of the invention contemplated by the invention are physiologically acceptable organic and inorganic salts.

The peptides of some embodiments of the invention are preferably utilized in a linear form, although it will be appreciated that in cases where cyclicization does not severely interfere with peptide characteristics, cyclic forms of the peptide can also be utilized.

According to specific embodiments the isolated peptide comprises a multimer. The multimer comprises at least two isolated peptide (e.g., 3 or 4), which may be identical or different peptides.

According to a specific embodiment the at least two isolated peptides are identical.

The term identical in this case refers to the chemical composition of the peptide per se.

According to another specific embodiment, the at least two isolated peptides are different. The term different as used in this case, refers to peptides having a different chemical composition. Thus the peptides may have different biological properties e.g., bind different targets or the same target in a different manner (e.g., difference in affinities).

According to specific embodiments, there is provided a fusion protein comprising at least one of the isolated peptide.

According to specific embodiment the agent is an isolated antibody.

According to specific embodiments the antibody comprises an antigen recognition domain capable of specifically binding an epitope on CDR3 of a TCR presented on a T cell. As used herein, the term “epitope” refers to any antigenic determinant on an antigen to which the paratope of an antibody or a TCR binds. Epitopic determinants usually consist of chemically active surface groupings of molecules such as amino acids or carbohydrate side chains and usually have specific three dimensional structural characteristics, as well as specific charge characteristics. According to a specific embodiment, the isolated antibody comprises an antigen recognition domain capable of specifically binding SEQ ID NO: 1 of a TCR presented on a T cell.

Antibodies, or immunoglobulins, comprise two heavy (H) chains linked together by disulfide bonds and two light (L) chains, each L chain being linked to a respective H chain by disulfide bonds in a “Y” shaped configuration. Proteolytic digestion of an antibody yields Fv (Fragment variable) and Fc (fragment crystalline) domains. The antigen binding domains, Fab's, include regions where the polypeptide sequence varies. The term F(ab′)₂ represents two Fab′ arms linked together by disulfide bonds. The central axis of the antibody is termed the Fc fragment. Each H chain has at its N-terminal end a variable (V) domain (V_{H}) followed by a number of constant (C) domains (C_{H}). Each L chain has a V domain (V_{L}) at one end and a C domain (C_{L}) at its other end, the V_{L} domain being aligned with the V_{H} domain and the C_{L} domain being aligned with the first C_{H} domain (C_{H}1). The V domains of each pair of L and H chains form the antigen-binding site. The domains on the L and H chains have the same general structure, and each domain comprises four framework regions (FRs), whose sequences are relatively conserved, joined by three hypervariable domains known as complementarity determining regions (CDR1-3). These domains contribute specificity and affinity of the antigen-binding site. The isotype of the H chain (gamma-γ, alpha-α, delta-δ, epsilon-ε or mu-μ) determines immunoglobulin class (IgG, IgA, IgD, IgE or IgM, respectively). There are several subtypes of IgG (IgG1, IgG2, IgG3, and IgG4). The L chain is either of two isotypes (kappa, κ or lambda, λ) found in all antibody classes.

The term “antibody” is used in the broadest sense and includes mAbs (including full-length or intact mAbs), polyclonal antibodies, multivalent antibodies, multi-specific antibodies (e.g., bispecific antibodies), and antibody fragments so long as they exhibit the desired biological activity.

The antibody according to the present invention is a molecule comprising at least the antigen-binding portion of an antibody. Antibody or antibodies according to the invention include intact antibodies, such as polyclonal antibodies or mAbs, as well as proteolytic fragments thereof such as Fab or F(ab′)₂ fragments. Further included within the scope of the invention are chimeric antibodies; human and humanized antibodies; recombinant and engineered antibodies, bi-specific antibodies, and fragments thereof. Furthermore, the DNA encoding the V region of the antibody can be inserted into the DNA encoding the C regions of other antibodies to produce chimeric antibodies. Single chain antibodies also fall within the scope of the present invention.

“Antibody fragments” comprise only a portion of an intact antibody, generally including an antigen binding site of the intact antibody and thus retaining the ability to bind antigen. Examples of antibody fragments encompassed by the present definition include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the C_{H}1 domain; (v) the Fv fragment having the V_{L} and V_{H} domains of a single arm of an antibody; (vi) the dAb fragment (Ward et al., Nature 1989, 341, 544-546) which consists of a V_{H} domain; (vii) isolated CDR regions; (viii) F(ab′)₂ fragments, a bivalent fragment including two Fab′ fragments linked by a disulfide bridge at the hinge region; (ix) single chain antibody molecules (e.g. single chain Fv; scFv) (Bird et al., Science 1988, 242, 423-426; and Huston et al., PNAS (USA) 1988, 85, 5879-5883); (x) “diabodies” with two antigen binding sites, comprising a V_{H} domain connected to a V_{L} domain in the same polypeptide chain (see, e.g., EP 404,097; WO 93/11161; and Hollinger et al., Proc. Natl. Acad. Sci. USA, 1993, 90, 6444-6448); (xi) “linear antibodies” comprising a pair of tandem Fd segments (V_{H}-C_{H}1-V_{H}-C_{H}1) which, together with complementary L chain polypeptides, form a pair of antigen binding regions (Zapata et al. Protein Eng., 1995, 8, 1057-1062; and U.S. Pat. No. 5,641,870).

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv).

Single-chain antibodies can be single-chain composite polypeptides having antigen binding capabilities and comprising amino acid sequences homologous or analogous to V_{H} and V_{L}, i.e., linked V_{H}-V_{L} or single-chain Fv (scFv).

The term “monoclonal antibody” as used herein refers to an antibody obtained from a population of substantially homogeneous antibodies, i.e., the individual antibodies comprising the population are identical except for possible naturally occurring mutations that may be present in minor amounts. MAbs are highly specific, being directed against a single antigen. Furthermore, in contrast to polyclonal antibody preparations that typically include different antibodies directed against different determinants (epitopes), each mAb is directed against a single determinant on the antigen. The modifier “monoclonal” is not to be construed as requiring production of the antibody by any particular method. mAbs may be obtained by methods known to those skilled in the art. For example, the mAbs to be used in accordance with the present invention may be made by the hybridoma method first described by Kohler et al., Nature 1975, 256, 495, or may be made by recombinant DNA methods (see, e.g., U.S. Pat. No. 4,816,567). The “monoclonal antibodies” may also be isolated from phage antibody libraries using the techniques described in Clackson et al., Nature 1991, 352, 624-628 or Marks et al., J. Mol. Biol., 1991, 222:581-597, for example. The mAbs may be isolated from a library from human lymphocytes and selected according to their specificity.

The mAbs of the present invention may be of any immunoglobulin class including IgG, IgM, IgE, IgA, and any subclass thereof. A hybridoma producing a mAb may be cultivated in vitro or in vivo. High titers of mAbs can be obtained by production in recombinant mammalian cells that contain the nucleic acids encoding the H and L chains of the mAb under the control of a cell-specific promoter. Such recombinant expresser cells are cultivated in large volumes in bioreactors. mAbs of any isotype are purified from culture supernatants, using filtration and column chromatography methods well known to those of skill in the art.

The mAbs herein specifically include “chimeric” antibodies in which a portion of the H and/or L chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)). In addition, CDR grafting may be performed to alter certain properties of the antibody molecule including affinity or specificity. A non-limiting example of CDR grafting is disclosed in U.S. Pat. No. 5,225,539.

Chimeric antibodies are molecules, the different portions of which are derived from different animal species, such as those having a variable region derived from a murine mAb and a human C region. Antibodies which have V region FR residues substantially from human antibody (termed an acceptor antibody) and CDRs substantially from a mouse antibody (termed a donor antibody) are also referred to as humanized antibodies. Chimeric antibodies are primarily used to reduce immunogenicity in application and to increase yields in production, for example, where murine mAbs have higher immunogenicity in humans (HAMA, which is human anti-mouse antibody response), such that human/murine chimeric mAbs are used. Chimeric antibodies and methods for their production are known in the art (for example PCT patent applications WO 86/01533, WO 97/02671, WO 90/07861, WO 92/22653 and U.S. Pat. Nos. 5,693,762, 5,693,761, 5,585,089, 5,530,101 and 5,225,539).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from CDRs of the recipient are replaced by residues from CDRs of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in either the recipient antibody or in the donor antibody. These modifications are made to further refine antibody performance, specificity, affinity and reduced immunogenicity. In general, the humanized antibody will comprise substantially all of at least one, and typically two, V domains, in which all or substantially all of the CDR loops correspond to those of a non-human immunoglobulin and all or substantially all of the FRs are those of a human immunoglobulin sequence. The humanized antibody optionally will also comprise at least a portion of an immunoglobulin C region (Fc), typically that of a human immunoglobulin in order to provide for a full mAb and appropriate effector functions as desired. For further details, see Jones et al., Nature 1986, 321, 522-525; Riechmann et al., Nature 1988, 332, 323-329; and Presta, Curr. Op. Struct. Biol., 1992 2, 593-596.

A “human antibody” is one which possesses an amino acid sequence which corresponds to that of an antibody produced by a human and/or encoded by the human genome and/or has been made using any of the techniques for making human antibodies as disclosed herein. This definition of a human antibody specifically excludes a humanized antibody comprising non-human CDR residues. Human antibodies can be produced using various techniques known in the art. In one embodiment, the human antibody is selected from a phage library, where that phage library expresses human antibodies (Vaughan et al. Nature Biotechnology 1996 14,309-314; Sheets et al. PNAS (USA), 1998, 95, 6157-6162); Hoogenboom and Winter, J. Mol. Biol., 1991, 227, 381; Marks et al., J. Mol. Biol., 1991, 222, 581). Human antibodies can also be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al, Bio/Technology 10: 779-783 (1992); Lonberg et al., Nature 368: 856-859 (1994); Morrison, Nature 368:812-13 (1994); Fishwild et al., Nature Biotechnology 14: 845-51 (1996); Neuberger, Nature Biotechnology 14: 826 (1996); Lonberg and Huszar, Intern. Rev. Immunol. 13:65-93 (1995). Alternatively, the human antibody may be prepared via immortalization of human B lymphocytes producing an antibody directed against a target antigen (such B lymphocytes may be recovered from an individual or may have been immunized in vitro) followed by screening with the antigen of interest for a specific antibody.

By the term “single-chain variable fragment (scFv)” is meant a fusion of the V_{H} and V_{L} regions, linked together with a short (usually serine, glycine) linker. Single-chain antibodies can be single chain composite polypeptides having antigen binding capabilities and comprising amino acid sequences homologous or analogous to V_{H} and V_{L} VL (linked V_{H}-V_{L} or single chain Fv (scFv)). Both V_{H} and V_{L} may copy natural mAb sequences or one or both of the chains may comprise a CDR-FR construct of the type described in U.S. Pat. No. 5,091,513, the entire contents of which are incorporated herein by reference. The separate polypeptides analogous to the V_{H} and V_{L} regions are held together by a polypeptide linker. Methods of production of such single chain antibodies, particularly where the DNA encoding the polypeptide structures of the V_{H} and V_{L} chains are known, may be accomplished in accordance with the methods described, for example, in U.S. Pat. Nos. 4,946,778, 5,091,513 and 5,096,815, the entire contents of each of which are incorporated herein by reference.

A “molecule having the antigen-binding portion of an antibody” as used herein is intended to include not only intact immunoglobulin molecules of any isotype and generated by any animal cell line or microorganism, but also the antigen-binding reactive fraction thereof, including, but not limited to, the Fab fragment, the Fab′ fragment, the F(ab′)₂ fragment, the variable portion of the heavy and/or light chains thereof, Fab mini-antibodies (see WO 93/15210, U.S. patent application Ser. No. 08/256,790, WO 96/13583, U.S. patent application Ser. No. 08/817,788, WO 96/37621, U.S. patent application Ser. No. 08/999,554, the entire contents of which are incorporated herein by reference), dimeric bispecific mini-antibodies (see Muller et al., 1998) and chimeric or single-chain antibodies incorporating such reactive fraction, as well as any other type of molecule or cell in which such antibody reactive fraction has been physically inserted, such as a chimeric T-cell receptor or a T-cell having such a receptor, or molecules developed to deliver therapeutic moieties by means of a portion of the molecule containing such a reactive fraction. Such molecules may be provided by any known technique, including, but not limited to, enzymatic cleavage, peptide synthesis or recombinant techniques.

Antibodies according to the invention can be obtained by administering a peptide, peptide analog, or cells expressing these, to an animal, preferably a nonhuman, using routine protocols. According to specific embodiment, the antibody of interest is obtained by a method comprising using the CDR3 peptides as described herein for producing or selecting an antibody specifically recognizing said peptide, thereby producing the antibody of interest. For preparation of Abs, any technique known in the art that provides antibodies produced by continuous cell line cultures can be used. Examples include various techniques, such as those in Kohler, G. and Milstein, C., Nature 256: 495-497 (1975); Kozbor et al., Immunology Today 4: 72 (1983); Cole et al., pg. 77-96 in Monoclonal Antibodies And Cancer Therapy, Alan R. Liss, Inc. (1985). According to specific embodiments, the antibodies are obtained by immunization of an animal.

Besides the conventional method of raising antibodies in vivo, antibodies can be generated in vitro using antibody display platforms such as, but not limited to, phage display, ribosome and mRNA display and microbial cell display technologies. Such a production of recombinant antibodies is much faster compared to conventional antibody production and they can be generated against an enormous number of antigens. Furthermore, when using the conventional method, many antigens prove to be non-immunogenic or extremely toxic, and therefore cannot be used to generate antibodies in animals. Moreover, affinity maturation (i.e., increasing the affinity and specificity) of recombinant antibodies is very simple and relatively fast. Finally, large numbers of different antibodies against a specific antigen can be generated in one selection procedure. To generate recombinant mAbs one can use various methods all based on display libraries to generate a large pool of antibodies with different antigen recognition sites. Such a library can be made in several ways: One can generate a synthetic repertoire by cloning synthetic CDR regions in a pool of H chain germline genes and thus generating a large antibody repertoire, from which recombinant antibody fragments with various specificities can be selected. One can use the lymphocyte pool of humans as starting material for the construction of an antibody library. It is possible to construct naive repertoires of human IgM antibodies and thus create a human library of large diversity. This method has been widely used successfully to select a large number of antibodies against different antigens. Protocols for bacteriophage library construction and selection of recombinant antibodies are provided in the well-known reference text Current Protocols in Immunology, Colligan et al (Eds.), John Wiley & Sons, Inc. (1992-2000), Chapter 17, Section 17.1.

Non-human antibodies may be humanized by any methods known in the art. In one method, the non-human CDRs are inserted into a human antibody or consensus antibody FR sequence. Further changes can then be introduced into the antibody framework to modulate affinity or immunogenicity.

For example, U.S. Pat. No. 5,585,089 of Queen et al. discloses a humanized immunoglobulin and methods of preparing same, wherein the humanized immunoglobulin comprises CDRs from a donor immunoglobulin and V_{H} and V_{L} region FRs from human acceptor immunoglobulin H and L chains, wherein said humanized immunoglobulin comprises amino acids from the donor immunoglobulin FR outside the Kabat and Chothia CDRs, wherein the donor amino acids replace corresponding amino acids in the acceptor immunoglobulin H or L chain frameworks.

U.S. Pat. No. 5,225,539, of Winter, also discloses an altered antibody or antigen-binding fragment thereof and methods of preparing same, wherein a V domain of the antibody or antigen-binding fragment has the FRs of a first immunoglobulin H or L chain V domain and the CDRs of a second immunoglobulin V_{H} or V_{L} domain, wherein said second immunoglobulin V_{H} or V_{L} domain is different from said first immunoglobulin V_{H} or V_{L} domain in antigen binding specificity, antigen binding affinity, stability, species, class or subclass.

The above-described antibodies can be employed to isolate or to identify clones expressing the polypeptides to purify the polypeptides by, for example, affinity chromatography.

Both neutralizing and activating antibodies are encompassed by the present invention.

According to a specific embodiment, the antibody is a neutralizing antibody.

A “neutralizing antibody” as used herein refers to an antibody capable of preventing, reducing, inhibiting or interfering the activity or signaling through a TCR, as determined by in vivo or in vitro assays, as per the specification, thereby suppressing activity of the T cell it binds to.

According to another specific embodiment, the antibody is an activating antibody.

An “activating antibody” as used herein refers to an antibody capable of eliciting activity or signaling through a TCR, as determined by in vivo or in vitro assays, as per the specification, thereby activating the T cell it binds to.

According to specific embodiments, the proteinaceous agents e.g., peptide of the present invention may be attached to a proteinaceous moiety which is heterologous to the CDR3 sequence (the heterologous sequence is not contiguously found in nature along with the CD3 sequence). Such a moiety may be an immunoglobulin fragment such as an Fc which is known to increase the bioavailability of protein based agents (e.g., peptides).

According to specific embodiments, the agent of the present invention may be attached to a non-proteinaceous moiety. It will be appreciated that the attachment of heterologous moieties, proteinaceous or non-proteinaceous, is contemplated herein for any agent used according to the present teachings. The elaboration of such a modification with respect to peptides should not be interpreted as limiting.

According to a specific embodiment the non-proteinaceous or proteinaceous moiety is a non-toxic moiety.

The phrase “non-proteinaceous moiety” as used herein refers to a molecule not including peptide bonded amino acids that is attached to the above-described peptide. Exemplary non-proteinaceous moieties which may be used according to the present teachings include, but are not limited to, polyethylene glycol (PEG), Polyvinyl pyrrolidone (PVP), poly(styrene comaleic anhydride) (SMA), and divinyl ether and maleic anhydride copolymer (DIVEMA). According to a specific embodiment, the non-proteinaceous moiety comprises polyethylene glycol (PEG).

Such a molecule is highly stable (resistant to in-vivo proteolytic activity probably due to steric hindrance conferred by the non-proteinaceous moiety) and may be produced using common solid phase synthesis methods which are inexpensive and highly efficient, as further described hereinbelow. However, it will be appreciated that recombinant techniques may still be used, whereby the recombinant peptide product is subjected to in-vitro modification (e.g., PEGylation as further described hereinbelow).

Bioconjugation of the agent e.g., peptide amino acid sequence, with PEG (i.e., PEGylation) can be effected using PEG derivatives such as N-hydroxysuccinimide (NHS) esters of PEG carboxylic acids, monomethoxyPEG₂-NHS, succinimidyl ester of carboxymethylated PEG (SCM-PEG), benzotriazole carbonate derivatives of PEG, glycidyl ethers of PEG, PEG p-nitrophenyl carbonates (PEG-NPC, such as methoxy PEG-NPC), PEG aldehydes, PEG-orthopyridyl-disulfide, carbonyldimidazol-activated PEGs, PEG-thiol, PEG-maleimide. Such PEG derivatives are commercially available at various molecular weights [See, e.g., Catalog, Polyethylene Glycol and Derivatives, 2000 (Shearwater Polymers, Inc., Huntsvlle, Ala.)]. If desired, many of the above derivatives are available in a monofunctional monomethoxyPEG (mPEG) form. In general, the PEG added to the peptide of the present invention should range from a molecular weight (MW) of several hundred Daltons to about 100 kDa (e.g., between 3-30 kDa). Larger MW PEG may be used, but may result in some loss of yield of PEGylated peptides. The purity of larger PEG molecules should be also watched, as it may be difficult to obtain larger MW PEG of purity as high as that obtainable for lower MW PEG. It is preferable to use PEG of at least 85% purity, and more preferably of at least 90% purity, 95% purity, or higher. PEGylation of molecules is further discussed in, e.g., Hermanson, Bioconjugate Techniques, Academic Press San Diego, Calif. (1996), at Chapter 15 and in Zalipsky et al., “Succinimidyl Carbonates of Polyethylene Glycol,” in Dunn and Ottenbrite, eds., Polymeric Drugs and Drug Delivery Systems, American Chemical Society, Washington, D.C. (1991).

Conveniently, PEG can be attached to a chosen position in the peptide by site-specific mutagenesis as long as the activity of the conjugate is retained. A target for PEGylation could be any Cysteine residue at the N-terminus or the C-terminus of the peptide sequence. Additionally or alternatively, other Cysteine residues can be added to the peptide amino acid sequence (e.g., at the N-terminus or the C-terminus) to thereby serve as a target for PEGylation. Computational analysis may be effected to select a preferred position for mutagenesis without compromising the activity.

Various conjugation chemistries of activated PEG such as PEG-maleimide, PEG-vinylsulfone (VS), PEG-acrylate (AC), PEG-orthopyridyl disulfide can be employed. Methods of preparing activated PEG molecules are known in the arts. For example, PEG-VS can be prepared under argon by reacting a dichloromethane (DCM) solution of the PEG-OH with NaH and then with di-vinylsulfone (molar ratios: OH 1:NaH 5:divinyl sulfone 50, at 0.2 gram PEG/mL DCM). PEG-AC is made under argon by reacting a DCM solution of the PEG-OH with acryloyl chloride and triethylamine (molar ratios: OH 1: acryloyl chloride 1.5: triethylamine 2, at 0.2 gram PEG/mL DCM). Such chemical groups can be attached to linearized, 2-arm, 4-arm, or 8-arm PEG molecules.

Resultant conjugated molecules (e.g., PEGylated or PVP-conjugated peptide) are separated, purified and qualified using e.g., high-performance liquid chromatography (HPLC) as well as biological assays.

The agents e.g., peptides of some embodiments of the invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

A preferred method of preparing the peptide compounds of some embodiments of the invention involves solid phase peptide synthesis.

Large scale peptide synthesis is described by Andersson Biopolymers 2000; 55(3):227-50.

Any of the proteinaceous agents described herein can be encoded from a polynucleotide. These polynucleotides can be used as therapeutics per se or in the recombinant production of the agent.

Thus, according to specific embodiments there is provided an isolated polynucleotide comprising a nucleic acid sequence encoding the agent, the peptide, or the antibody of the present invention.

The phrase “an isolated polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

The isolated polynucleotide may be ligated into an expression construct which can be used as a shuttle vector (for the mere replication of the polynucleotide) or an expression vector whereby the isolated polynucleotide is typically ligated in a cis-acting manner to a cis acting element such as a promoter.

Such an expression vectors can be typically classified as viral vectors and bacterial vectors. The term “viral vector” or “bacterial vector” refers to a virus or bacteria, respectively, which can be administered to a human host without causing any disease or pathology and which encodes a protein or peptide or epitope not present in the native virus of bacteria. Such viral and bacterial vectors can be readily produced by recombinant methods well known in the art. Non-limiting examples include poxviruses, adenoviruses, alphaviruses, lentiviruses, Listeria monocytogenes, Salmonella typhi, Fibrio cholerae, Shigella sonnei, Mycobacterium bovis, and Bacillus anthracis.

The term “nucleic acid” in the context of vaccine refers to the injection of DNA to the host, whereby DNA is taken up by cells, transcribed and translated to protein or peptide that is presented to the immune system and thus elicit antibody- and cell-based immune responses specific to the peptide of interest. Non-limiting examples of such nucleic acid vaccines are purified nucleic acid administered alone, DNA-liposome complexes, DNA-coated polymers, and metal-coated DNA.

The agents, the peptides, the antibodies and polynucleotides of the present invention can be used to treat a disease associated with a T cell expressing a specific CDR3-TCR.

Thus, according to specific embodiments, (i) the isolated agent; (ii) the isolated peptide; or (iii) an isolated peptide of no more than 20 amino acids comprising an amino acid sequence having a CDR3 sequence of a TCR on a T cell, said CDR3 sequence being selected from the group consisting of SEQ ID NOs of Table 8, is used in the manufacture of a medicament identified for treating a disease associated with the T cell.

According to specific embodiments, there is provided a method of treating a disease associated with the T cell, the method comprising administering to a subject in need thereof an effective amount of: (i) the isolated agent; (ii) the isolated peptide; or (iii) an isolated peptide of no more than 20 amino acids comprising an amino acid sequence having a CDR3 sequence of a TCR on a T cell, said CDR3 sequence being selected from the group consisting of SEQ ID NOs of Table 8, thereby treating the disease associated with the T cell.

According to other specific embodiments, the isolated antibody is used in the manufacture of a medicament identified for treating a disease associated with said T cell.

According to specific embodiments, there is provided a method of treating a disease associated with a T cell expressing a TCR-CDR3 segment comprising an amino acid sequence of SEQ ID NO: 1 in a subject in need thereof, the method comprising administering to the subject a therapeutically effective amount of the isolated antibody of claim 7, thereby treating the disease associated with a T cell expressing said TCR-CDR3 segment comprising an amino acid sequence of SEQ ID NO: 1 in the subject.

As used herein, the term “disease associated with a T cell” refers to a pathological condition which onset or progression is associated with under activity or over activity of T cells expressing a specific CDR3. The T cell may be an effector T cell or a regulatory T cell.

According to specific embodiments, wherein the disease is associated with activity of the T cell then the immune response induced by the agent is dowregulation of the activity.

According to other specific embodiments, wherein the disease is associated with suppression of activity of the T cell then the immune response induced by the agent is upregulation of the activity.

This includes chronic and acute disorders or diseases including those pathological conditions which predispose the mammal to the disorder in question. Non-limiting examples of disorders to be treated herein include cancer e.g. benign and malignant tumors; leukemias and lymphoid malignancies; autoimmune diseases, graft rejection disease (e.g. graft vs. host disease), neuronal, glial, astrocytal, hypothalamic and other glandular, macrophagal, epithelial, stromal and blastocoelic disorders; and inflammatory, angiogenic, immunologic disorders or hyperpermeability states.

Typically, activating the public clonotype is good for autoimmune disease therapy, and inactivating the public clonotype is likely to be good for tumor immunotherapy. The experimental results demonstrated herein for the first time demonstrate that it should be possible to unleash a tumor-specific “autoimmune” response by administering antibodies to the public CDR3 TCR peptides of tumor-infiltrating T cells.

As used herein, the term “subject” includes mammals, preferably human beings at any age which suffer from or is at risk of the disease.

“Treatment” refers to both therapeutic treatment and prophylactic or preventative measures. Those in need of treatment include those already with the disorder as well as those in which the disorder is to be prevented.

Immunotherapy of tumor growth was approached by the use of anti PD1 antibodies demonstrating immune regulatory mechanism to attack tumor cells. To overcome some lack of specificity, use of T cells expressing TCR molecules which share common CDR3 could induce an autoimmune reaction specific to the tumor cells.

Thus, according to specific embodiments, the disease is cancer.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include squamous cell cancer, lung cancer (including small-cell lung cancer, non-small-cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high-grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. Preferably, the cancer is selected from the group consisting of breast cancer, colorectal cancer, rectal cancer, non-small cell lung cancer, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, and multiple myeloma. The cancerous conditions amenable for treatment of the invention include metastatic cancers.

According to specific embodiments, the cancer is lung cancer.

According to a specific embodiment, the cancer is lung carcinoma.

According to specific embodiments, the disease is an autoimmune disease. Specific examples of autoimmune diseases which may be treated according to the teachings of the present invention include, but are not limited to, rheumatoid diseases, rheumatoid autoimmune diseases, rheumatoid arthritis (Krenn V. et al., Histol Histopathol 2000 July; 15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2):49), sclerosis, systemic sclerosis (Renaudineau Y. et al., Clin Diagn Lab Immunol. 1999 March; 6 (2):156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29 (2):339), thyroiditis, spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), repeated fetal loss (Tincani A. et al., Lupus 1998; 7 Suppl 2:S107-9), neurodegenerative diseases, neurological diseases, neurological autoimmune diseases, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83), motor neuropathies (Kornberg A J. J Clin Neurosci. 2000 May; 7 (3):191), Guillain-Barre syndrome, neuropathies and autoimmune neuropathies (Kusunoki S. Am J Med Sci. 2000 April; 319 (4):234), myasthenic diseases, Lambert-Eaton myasthenic syndrome (Takamori M. Am J Med Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann N Y Acad Sci. 1998 May 13; 841:482), Chronic obstructive pulmonary disease (COPD), cardiovascular diseases, cardiovascular autoimmune diseases, atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), granulomatosis, Wegener's granulomatosis, arteritis, Takayasu's arteritis and Kawasaki syndrome (Praprotnik S. et al., Wien Klin Wochenschr 2000 Aug. 25; 112 (15-16):660); anti-factor VIII autoimmune disease (Lacroix-Desmazes S. et al., Semin Thromb Hemost. 2000; 26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). 2000 May; 151 (3):178); antiphospholipid syndrome (Flamholz R. et al., J Clin Apheresis 1999; 14 (4):171); heart failure, agonist-like beta-adrenoceptor antibodies in heart failure (Wallukat G. et al., Am J Cardiol. 1999 Jun. 17; 83 (12A):75H), thrombocytopenic purpura (Moccia F. Ann Ital Med Int. 1999 April-June; 14 (2):114); hemolytic anemia, autoimmune hemolytic anemia (Efremov D G. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefuah 2000 Jan. 16; 138 (2):122), Crohn's disease, ulcerative colitis, psoriasis autoimmune diseases of the musculature, myositis, autoimmune myositis, Sjogren's syndrome (Feist E. et al., Int Arch Allergy Immunol 2000 September; 123 (1):92); smooth muscle autoimmune disease (Zauli D. et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P. et al., Eur J Gastroenterol Hepatol. 1999 June; 11 (6):595).

According to specific embodiments, the autoimmune disease is selected from the group consisting of rheumatoid arthritis, multiple sclerosis, type-1 diabetes, Chronic obstructive pulmonary disease (COPD), Crohn's disease, ulcerative colitis, and psoriasis.

According to other specific embodiments, the disease is a transplantation related disease i.e. graft rejection disease.

Specific examples of transplantation-related diseases which may be treated according to the teachings of the present invention include but are not limited to host vs. graft disease, chronic graft rejection, subacute graft rejection, hyperacute graft rejection, acute graft rejection, allograft rejection, xenograft rejection and graft-versus-host disease (GVHD).

According to specific embodiments the graft rejection disease is host vs. graft disease.

According to other specific embodiments, the disease is pathogenic disease. Specific examples of intracellular pathogens infections which may be treated according to the teachings of the present invention include, but are not limited to, infections by viral pathogens, intracellular mycobacterial pathogens (such as, for example, Mycobacterium tuberculosis), intracellular bacterial pathogens (such as, for example, Listeria monocytogenes), or intracellular protozoan pathogens (such as, for example, Leishmania and Trypanosoma).

Specific types of viral pathogens causing infectious diseases treatable according to the teachings of the present invention include, but are not limited to, retroviruses, circoviruses, parvoviruses, papovaviruses, adenoviruses, herpesviruses, iridoviruses, poxviruses, hepadnaviruses, picornaviruses, caliciviruses, togaviruses, flaviviruses, reoviruses, orthomyxoviruses, paramyxoviruses, rhabdoviruses, bunyaviruses, coronaviruses, arenaviruses, and filoviruses.

Specific examples of viral infections which may be treated according to the teachings of the present invention include, but are not limited to, human immunodeficiency virus (HIV)-induced acquired immunodeficiency syndrome (AIDS), influenza, rhinoviral infection, viral meningitis, Epstein-Barr virus (EBV) infection, hepatitis A, B or C virus infection, measles, papilloma virus infection/warts, cytomegalovirus (CMV) infection, Herpes simplex virus infection, yellow fever, Ebola virus infection, rabies, etc.

According to specific embodiments, the pathogenic disease is human immunodeficiency virus or tuberculosis infection.

The isolated agents, peptides antibodies and polynucleotides of the present invention can be used to treat a disease or a condition associated with a pathological T cell alone or in combination with other established or experimental therapeutic regimen for such disorders. Thus for example, antibodies can be used in combination with an anti-neoplastic composition. The term “anti-neoplastic composition” refers to a composition useful in treating cancer comprising at least one active therapeutic agent capable of inhibiting or preventing tumor growth or function or metastasis, and/or causing destruction of tumor cells. Therapeutic agents suitable in an anti-neoplastic composition for treating cancer include, but not limited to, chemotherapeutic agents, radioactive isotopes, toxins, cytokines such as interferons, and antagonistic agents targeting cytokines, cytokine receptors or antigens associated with tumor cells. For example, therapeutic agents useful in the present invention can be antibodies such as anti-HER2 antibody and anti-CD20 antibody, or small molecule tyrosine kinase inhibitors such as VEGF receptor inhibitors and EGF receptor inhibitors. Preferably the therapeutic agent is a chemotherapeutic agent.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem Intl. Ed. Engl. 33:183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′, 2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOLR® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISORR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in aberrant cell proliferation, such as, for example, PKC-alpha, Raf and H-Ras; ribozymes such as a VEGF expression inhibitor (e.g., ANGIOZYME® ribozyme) and a HER2 expression inhibitor; vaccines such as gene therapy DNA-based vaccines, for example, ALLOVECTIN® vaccine, LEUVECTIN® vaccine, and VAXID® vaccine; PROLEUKIN®rIL-2; LURTOTECAN® topoisomerase 1 inhibitor; ABARELIX® rmRH; and pharmaceutically acceptable salts, acids or derivatives of any of the above.

Qualification of the agents, peptides, antibodies and polynucleotides of the resent invention for the treatment of a specific disease may be effected by testing them in a typical animal model.

Typically shared, relatively public or public CDR3 sequences, that have been annotated to be associated with a specific disease or are suspected to be involved in a specific disease are selected after confirming that they are also relatively public or public in human TCR repertoires. These peptides are synthesized using methods known in the art, typically automated solid-phase synthesis, purified and used to immunize rabbits in order to obtain high-titer antibodies.

Thus, for example, public CDR3 peptides reactive with known tumor-associated antigens such as MDM2 and HSP60 or expressed by tumor-infiltrating T cells and shared by mice and humans, can be synthesized based on the high-throughput screening results and the dataset of public annotated CDR3 sequences.

The respective agent is chosen e.g. isolated peptide, antibody, T cell or polynucleotide and produced by any method known in the art as further disclosed hereinabove. In the next step, the effect of administering the agent to an animal model is evaluated. Non-limiting examples of animal models that can be used include syngeneic tumor models such as the B16 melanoma in both local, subcutaneous growth and dispersed lung seeding, the 3LL tumor in local growth with spontaneous metastasis to the lung and in intravenous dispersal to the lungs and the GL261 Glioblastoma.

Thus, for example, the effect of administering CDR3 antibodies or control antibodies to mice bearing tumors is studied aiming to achieve tumor rejection or inhibition of tumor growth. The mice are followed and evaluated for tumor growth and tumor spread. In addition, the anti-tumor T cell and B cell immune responses responsible for tumor rejection are characterized by determining cytotoxic T cell reactions and serum antibody responses to the tumor cells in vitro; and adoptive transfer of T cells from antibody-treated mice to naïve mice, which is then challenged with the tumors. Specific sets of monoclonal and humanized antibodies can then be developed for unleashing controlled anti-tumor “autoimmunity” as an effective tumor immunotherapy. The optimal doses and dose schedules of the antibodies are then determined for anti-CDR3 antibodies showing positive results in the tumor models.

The agent, the isolated peptide, antibody or polynucleotide can be administered to the subject per se, or in a pharmaceutical composition where each or both are mixed with suitable carriers or excipients.

According to specific embodiments, the pharmaceutical composition further comprises an adjuvant or a delivery system.

As used herein a “pharmaceutical composition” refers to a preparation of the active ingredient described herein with other chemical components such as physiologically suitable carriers and excipients. The purpose of a pharmaceutical composition is to facilitate administration of a compound to an organism.

Pharmaceutical compositions comprising peptides and analog or antibodies or fragments thereof, are disclosed in the present invention, together with novel formulations, for use in prevention, suppression or treatment of a disease associated with a T cell expressing a specific CDR3-TCR, as disclosed hereinabove.

Herein the term “active ingredient” refers to the isolated agent, peptide, antibody and/or the polynucleotide accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Apart from other considerations, the fact that some of the novel active ingredients of the invention are peptides, peptide analogs or peptidomimetics, dictates that the formulation be suitable for delivery of these type of compounds. In general, peptides are less suitable for oral administration due to susceptibility to digestion by gastric acids or intestinal enzymes, but it is now disclosed that the compositions according to the present invention are also suitable for oral administration. Other routes of administration according to the present invention are intra-articular, intravenous, intramuscular, subcutaneous, intradermal, or intrathecal.

Pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or lyophilizing or liposome capturing processes.

Pharmaceutical compositions for use in accordance with the present invention thus may be formulated in conventional manner using one or more physiologically acceptable carriers comprising excipients and auxiliaries, which facilitate processing of the active compounds into preparations which, can be used pharmaceutically. Proper formulation is dependent upon the route of administration chosen.

For injection, the compounds of the invention may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants for example polyethylene glycol are generally known in the art.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for the chosen route of administration. For buccal administration, the compositions may take the form of tablets or lozenges formulated in conventional manner.

For administration by inhalation, the variants for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the peptide and a suitable powder base such as lactose or starch.

Pharmaceutical compositions for parenteral administration include aqueous solutions of the active ingredients in water-soluble form. Additionally, suspensions of the active compounds may be prepared as appropriate oily injection suspensions. Suitable natural or synthetic carriers are well known in the art (Pillai et al., Curr. Opin. Chem. Biol. 5, 447, 2001). Optionally, the suspension may also contain suitable stabilizers or agents, which increase the solubility of the compounds, to allow for the preparation of highly concentrated solutions. Alternatively, the active ingredient may be in powder form for reconstitution with a suitable vehicle, e.g., sterile, pyrogen-free water, before use.

The compounds of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. As used herein, the phrase “therapeutically effective amount” refers to an amount of active ingredient effective to prevent, delay, alleviate or ameliorate symptoms of a disease of the subject being treated or prolong the survival of the subject being treated. Determination of a therapeutically effective amount is well within the capability of those skilled in the art, especially in light of the detailed disclosure provided herein.

In the case of cancer, the therapeutically effective amount may reduce the number of cancer cells; reduce the tumor size; inhibit (i.e., slow to some extent and preferably stop) local cancer cell growth, inhibit cancer cell infiltration into peripheral organs; inhibit tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in-vitro, in cell cultures or experimental animals, e.g., by determining the IC50 (the concentration which provides 50% inhibition) and the LD50 (lethal dose causing death in 50% of the tested animals) for a subject compound. The data obtained from these in-vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition (e.g. Fingl, et al., 1975, in “The Pharmacological Basis of Therapeutics”, Ch. 1 p. 1).

Dosage amount and interval may be adjusted individually to provide levels of the active ingredient that are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can also be a single administration of a slow release composition, with course of treatment lasting from several days to several weeks or until cure is effected or diminution of the disease state is achieved. The amount of a composition to be administered will, of course, be dependent on the subject being treated, the severity of the affliction, the manner of administration, the judgment of the prescribing physician, and all other relevant factors.

In one particularly preferred embodiment according to the present invention, the active ingredients are administered orally (e.g. as a syrup, capsule, or tablet).

In certain embodiments, delivery of the active ingredient can be enhanced by the use of protective excipients. This is typically accomplished either by complexing the active ingredient with a composition to render it resistant to acidic and enzymatic hydrolysis or by packaging the active ingredient in an appropriately resistant carrier such as a liposome. Attempts of protecting e.g. polypeptides for oral delivery have been published (e.g., U.S. Pat. Nos. 8,093,207, 7,666,446 and 7,316,819).

Elevated serum half-life can be maintained by the use of sustained-release protein “packaging” systems. Such sustained release systems are well known to those of skill in the art. In one preferred embodiment, the ProLease biodegradable microsphere delivery system for proteins and peptides (Tracy, 1998, Biotechnol. Prog. 14, 108; Johnson et al., 1996, Nature Med. 2, 795; Herbert et al., 1998, Pharmaceut. Res. 15, 357) a dry powder composed of biodegradable polymeric microspheres containing the protein in a polymer matrix that can be compounded as a dry formulation with or without other agents.

The foregoing formulations and administration methods are intended to be illustrative and not limiting. It will be appreciated that, using the teaching provided herein, other suitable formulations and modes of administration can be readily devised.

Formulations of the present invention suitable for oral administration may be presented as discrete units such as capsules, cachets, tablets, lozenges comprising the peptide(s) in a flavoured base, usually sucrose and acacia and tragacanth; pastilles comprising the active ingredient(s) in an inert base such as gelatin and glycerin, or sucrose and acacia; and mouth washes comprising the active ingredient(s) in a suitable liquid carrier. Each formulation generally contains a predetermined amount of the active peptide(s); as a powder or granules; or a solution or suspension in an aqueous or non-aqueous liquid such as a syrup, an elixir, an emulsion or draught and the like.

A tablet may be made by compression or moulding, optionally with one or more accessory ingredients. Compressed tablets may be prepared by compressing in a suitable machine the active peptide(s) in a free-flowing form such as a powder or granules, optionally mixed with a binder, (eg povidone, gelatin, hydroxypropylmethyl cellulose), lubricant, inert diluent, preservative, disintegrant (e.g. sodium starch glycollate, cross-linked povidone, cross-linked sodium carboxymethyl cellulose), surface active or dispersing agent. Moulded tablets may be made by moulding in a suitable machine a mixture of the powdered peptide(s) moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile.

A syrup may be made by adding the active ingredient(s) to a concentrated, aqueous solution of a sugar, for example, sucrose, to which may also be added any necessary ingredients. Such accessory ingredients) may include flavourings, an agent to retard crystallisation of the sugar or an agent to increase the solubility of any other ingredients, such as a polyhydric alcohol, for example, glycerol or sorbitol.

In addition to the aforementioned ingredients, the formulations of this invention may further include one or more accessory ingredient(s) selected from diluents, buffers, flavouring agents, binders, surface active agents, thickeners, lubricants, preservatives, (including antioxidants) and the like.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The following examples are intended to illustrate how to make and use the compounds and methods of this invention and are in no way to be construed as a limitation. Although the invention will now be described in conjunction with specific embodiments thereof, it is evident that many modifications and variations will be apparent to those skilled in the art. Accordingly, it is intended to embrace all such modifications and variations that fall within the spirit and broad scope of the amended claims.

### EXAMPLES

### Methods

### Library Preparation for TCR-seq

Libraries were prepared and pre-processed as published (Ndifon et al. 2012, Proc Natl Acad Sci USA 109, 39, 15865-15870). Briefly, total RNA was extracted from T cells using RNeasy Mini Kit (Qiagen, Hilden, Germany). The RNA was then reverse transcribed using SuperScript II reverse transcriptase (RT) enzyme (Invitrogen, La Jolla, Calif.). The primer for the RT reaction was a TCR Cβ-specific primer linked to the 3′-end Illumina sequencing adapter. The resulting cDNA was then amplified using PCR (Phusion; Finnzymes) with a Cβ-3′adp primer and Vβ-specific 5′ primers. Each Vβ-specific primer was anchored to a restriction site sequence for the ACUI restriction enzyme. PCR products were then cleaned using QIAquick PCR purification kit (Qiagen, Hilden, Germany), followed by enzymatic digestion with ACUI enzyme (New England BioLabs, Ipswich, Mass.). Then, the 5′Illumina adaptor (dsDNA, with NN overhang) were ligated (T4 ligase; Fermentas, Vilnius, Lithuania). The adaptors also contained 3-nucleotide long tags for multiplexing of samples to the same Illumina sequencing run. A second round of PCR amplification was performed, using universal primers for the 5′ and 3′ Illumina adapters. Final PCR products were run on a 2% agarose gel, cut at the desired length (˜250 bp), and purified using Wizard SV Gel and PCR Clean-Up System (Promega, Madison, Wis.) to produce the final library. The libraries were sequenced using Genome Analyzer II or HiSeq2000 (Illumina).

### Production of Anti-C9-CDR3 Antibodies

Polyclonal antibodies were raised in rabbits (by EZBiolab Inc., USA) against the C9 peptide (SEQ ID NO: 1). Protein A/G agarose beads were used for antibody purification. Serum of un-immunized rabbits was used as a control (pre-immune serum, also after purification).

### In-Vive Lung Tumor Model

20 C57BL/6 mice were injected intravenous (iv) with 5×10⁵ cells of a syngeneic mouse Lewis lung carcinoma cell line (D-122). Mice were divided into 2 groups, 10 animals in each group. On the following day, the mice of group 1 were injected inter-peritonealy (ip) with 100 μg of control pre-immune serum and the mice of group 2 were injected ip with 100 μg of polyclonal anti-C9-CDR3 antibody. 10 days later mice were boosted with another 100 μg of the control serum or the polyclonal anti-C9-CDR3 antibody, respectively. One month post tumor injection all mice were sacrificed and tumor nodules in lungs were counted.

### Example 1: Pre-Processing and Error Correction for Raw Reads

Raw reads containing bases with Q-value≦30 were filtered out, and then the remaining reads were separated according to their barcodes. Next, the reads were aligned to each of the germline Vβ/Jβ gene segments from IMGT (Lefiranc et al. 2009, Nucleic Acids Res 37 (Database issue): D1006-1012) using the Smith-Waterman algorithm. Each read was assigned its best-aligning Vβ/Jβ if the number of matching nucleotides (alignment length) was above a threshold, 11 nt for Vβ, 9 nt for Jβ. To reduce the effect of sequencing errors, hierarchical clustering to group reads assigned the same Vβ and Jβ genes and are with an edit distance less than 2 were used. Then, the sequences were annotated by matching the D3 to the junction, identifying deleted/inserted nucleotides and elongated the read to its full CDR30 length (by IMGT convention). Finally, the nt sequences were translated into amino-acid (aa) CDR30. Only sequences that are in-frame (i.e. no stop codons), have a copy number of at least 2 and have less than 2 bp enzyme cleavage error, were used. These are referred to as annotated reads (Table 1). The copy-number was also corrected, to adjust for PCR and sub-sampling bias, as published in Ndifon 2012 ibid.

TABLE 1
Samples' summary

| | | VJ assigned | Unique | Unique AA |
|---|---|---|---|---|
| Sample | Immunization | reads* | reads (nt) | sequences |
| 1 | Untreated | 86,523 | 22,838 | 18,063 |
| 2 | Untreated | 53,026 | 24,632 | 19,671 |
| 3 | Untreated | 64,654 | 22,004 | 17,779 |
| 4 | Untreated | 101,075 | 41,323 | 31,407 |
| 5 | Untreated | 216,533 | 78,613 | 58,612 |
| 6 | Untreated | 27,045 | 12,507 | 10,784 |
| 7 | Untreated | 91,998 | 32,683 | 25,807 |
| 8 | Untreated | 38,729 | 20,329 | 18,248 |
| 9 | Untreated | 155,857 | 60,678 | 49,388 |
| 10 | Untreated | 32,251 | 18,903 | 15,531 |
| 11 | Untreated | 42,585 | 19,113 | 15,403 |
| 12 | Untreated | 137,894 | 55,037 | 40,455 |
| 13 | CFA | 84,731 | 38,774 | 32,133 |
| 14 | CFA | 123,187 | 42,970 | 33,300 |
| 15 | CFA | 156,212 | 69,699 | 54,460 |
| 16 | CFA | 63,832 | 25,063 | 21,563 |
| 17 | CFA | 92,015 | 32,286 | 27,335 |
| 18 | CFA | 106,855 | 43,474 | 35,601 |
| 19 | CFA | 24,583 | 14,358 | 12,229 |
| 20 | CFA + OVA | 156,762 | 53,669 | 43,152 |
| 21 | CFA + OVA | 105,386 | 42,989 | 34,224 |
| 22 | CFA + OVA | 52,878 | 21,613 | 18,441 |
| 23 | CFA + OVA | 52,302 | 19,571 | 16,889 |
| 24 | CFA + OVA | 118,978 | 45,789 | 38,195 |
| 25 | CFA + OVA | 104,616 | 44,567 | 37,112 |
| 26 | CFA + OVA | 40,477 | 22,413 | 18,129 |
| 27 | CFA + OVA | 21,267 | 11,784 | 9,888 |
| 28 | CFA + OVA | 67,123 | 31,016 | 24,185 |

CFA—complete Freund's adjuvant
OVA—ovalbumine
*VJ assigned reads = reads which were unambiguously mapped to V and J segments, have a consensus CDR3 and encode for a sense (in-frame) CDR3 amino acid sequence. Also, we filtered out reads with cleavage error >2 or were found only once in a sample, to minimize noise (see methods). The copy number shown here is normalized to account for PCR biases and effects of experimental sub-sampling of library material, as published (Ndifon et al. 2012 ibid).

Most of the CDR3 aa sequences were found in only one mouse (˜69% of all sequences). However, hundreds of sequences were highly shared among individual mice; 1,908 sequences were shared by more than 75% (n>21) of the mice (Table 10). Notably, 289 CDR3 aa sequences were found that were shared by all 28 mice (˜0.08% of all sequences) (Table 10).

TABLE 10
The relatively public and public CDR3 amino acid sequences-1,908 CDR3 sequences shared by more than 75% (n > 21) of the
mice tested

| SEQ | | sharing | SEQ | | sharing | SEQ | | sharing | SEQ | | SEQ |
|---|---|---|---|---|---|---|---|---|---|---|---|
| ID | | level | ID | | level | ID | | level | ID | | ID |
| NO: | sequence | in mice | NO: | sequence | in mice | NO: | sequence | in mice | NO: | sequence | NO: |
| 1925 | CASSLGGNQDTQYF | 27 | 746 | CASSLGVNQDTQYF | 27 | 1142 | CASSPGTTNERLFF | 25 | 352 | CASSFKDTQFY | 23 |
| 20 | CASSLGGQNTLYF | 28 | 663 | CASGDSSAETLYF | 27 | 1102 | CASSLQGDQDTQYF | 25 | 1498 | CASSFTGGQNTLYF | 23 |
| 21 | CASSLGNSDYTF | 28 | 146 | CASGDADTQYF | 27 | 1097 | CASSLGVNYAEQFF | 25 | 1470 | CASRDSGNTLYF | 23 |
| 2 | CASSGTGQDTQYF | 27 | 709 | CASSLDREVFF | 27 | 1203 | CASSSQGSAETLYF | 25 | 1655 | CASSTGGNYAEQFF | 23 |
| 22 | CASSSANSDYTF | 27 | 147 | CASSLVGYEQYF | 27 | 1197 | CASSSGQGNTEVFF | 25 | 1578 | CASSPGLGQNTLYF | 23 |
| 23 | CASSSGNSDYTF | 27 | 779 | CASSPGQGAETLYF | 27 | 1088 | CASSLGTSNERLFF | 25 | 1597 | CASSPTVNQDTQYF | 23 |
| 24 | CASSGTANTEVFF | 26 | 822 | CASSSGQQNTLYF | 27 | 1096 | CASSLGTSSAETLYF | 25 | 1580 | CASSPGNQDTQYF | 23 |
| 25 | CASSGQGNYAEQFF | 26 | 701 | CASSLASQNTLYF | 27 | 1115 | CASSLTGENTLYF | 25 | 1582 | CASSPGQGNERLFF | 23 |
| 3 | CASSGTGEDTQYF | 25 | 732 | CASSLGGNTGQLYF | 27 | 1052 | CASSFGTANTEVFF | 25 | 1641 | CASSRTISNERLFF | 23 |
| 26 | CASGDWGYEQYF | 25 | 698 | CASSLAGGYAEQFF | 27 | 1030 | CASGSAETLYF | 25 | 1487 | CASSDSSYNSPLYF | 23 |
| 27 | CASGDAGGSYEQYF | 25 | 759 | CASSLRGQNTLYF | 27 | 1027 | CASGEGGNTLYF | 25 | 1489 | CASSDWGQDTQYF | 23 |
| 28 | CASSDGANTEVFF | 24 | 704 | CASSLDNSDYTF | 27 | 1139 | CASSRNTEVFF | 25 | 353 | CASSDEGYEQYF | 23 |
| 4 | CASSGLEDTQYF | 24 | 829 | CSAGGQNTLYF | 27 | 1175 | CASSLQGANTGQLYF | 25 | 1475 | CASSDAETLYF | 23 |
| 29 | CASSHSGNTLYF | 23 | 702 | CASSLDAETLYF | 27 | 1101 | CASSLQGANTGQLYF | 25 | 354 | CASSDADTQYF | 23 |
| 30 | CASSGTDQDTQYF | 23 | 741 | CASSLGQGYAEQFF | 27 | 247 | CASSLLGQDTQYF | 25 | 1541 | CASSLLGNTGQLYF | 23 |
| 5 | CASGGYEQYF | 23 | 789 | CASSQDSSGNTLYF | 27 | 1009 | CASGDAGAEQFF | 25 | 1469 | CASGTGNYAEQFF | 23 |
| 31 | CASSPGQSNERLFF | 23 | 810 | CASSRDWGNAEQFF | 27 | 1014 | CASGDAGNTEVFF | 25 | 1462 | CASGDWGAETLFY | 23 |
| 32 | CASSRTANTGQLYF | 23 | 796 | CASSRAETLYF | 27 | 248 | CASSLDGAEQFF | 25 | 1447 | CASGDATTSAETLYF | 23 |
| 33 | CASSDSANTEVFF | 22 | 148 | CASGDEDTQYF | 27 | 1033 | CASRDSSGNTLYF | 25 | 1443 | CASGDAGVQDTQYF | 23 |
| 34 | CASSLEGDTEVFF | 22 | 672 | CASRDSSQNTLYF | 27 | 1043 | CASSDRGAETLYF | 25 | 1464 | CASGDWGQGTQYF | 23 |
| 639 | CASSSQGAETLYF | 28 | 767 | CASSLTTNTEVFF | 27 | 1031 | CASRDNYAEQFF | 25 | 355 | CASSPDQDTQYF | 23 |
| 509 | CASSLESANSDYTF | 28 | 763 | CASSLTANTGQLYF | 27 | 1147 | CASSPTVSNERLFF | 25 | 1619 | CASSRDTQDTQYF | 23 |
| 35 | CASSLEGEDTQYF | 28 | 803 | CASSRDRGNTLYF | 27 | 249 | CASSQDRDSDYTF | 25 | 356 | CASSPGGQDTQYF | 23 |
| 468 | CASSFSAETLYF | 28 | 724 | CASSLEGTGQLYF | 27 | 1155 | CCASSQGQNTLYF | 25 | 1614 | CASSRDNNQAPLF | 23 |
| 594 | CASSPTGSQNTLYF | 28 | 772 | CASSPAETLYF | 27 | 1165 | CASSRDRANSDYTF | 25 | 1553 | CASSLQGNQAPLF | 23 |
| 570 | CASSPDSAETLYF | 28 | 781 | CASSPGSAETLYF | 27 | 1042 | CASSDNTEVFF | 25 | 1549 | CASSLQGGAETLYF | 23 |
| 36 | CASSPGQQDTQYF | 28 | 788 | CASSQDSAETLYF | 27 | 1041 | CASSDNSQNTLYF | 25 | 1558 | CASSLRGGQNTLYF | 23 |
| 608 | CASSRDNQDTQYF | 28 | 149 | CASSQGQYEQYF | 27 | 1169 | CASSRDTEVF | 25 | 357 | CASSLRQYEQYF | 23 |
| 520 | CASSLGGSAETLYF | 28 | 787 | CASSPTGGQNTLYF | 27 | 1107 | CASSLSGAETLYF | 25 | 1539 | CASSLGTTNERLFF | 23 |
| 37 | CASSFQDTQYF | 28 | 786 | CASSPTANTEVFF | 27 | 1153 | CASSQGGQNTLFY | 25 | 1450 | CASGDGNTEVFF | 23 |
| 528 | CASSLGQNTLYF | 28 | 150 | CASSLDSSYEQYF | 27 | 1064 | CASSLAGDQDTQYF | 25 | 1573 | CASSLVTGQLYF | 23 |
| 640 | CASSSQNTLYF | 28 | 151 | CASSPTGYEQYF | 27 | 1108 | CASSLSGNYAEQFF | 25 | 358 | CASSLVSYEQYF | 23 |
| 472 | CASSHSAETLYF | 28 | 776 | CASSPGLGNYAEFF | 27 | 1111 | CASSLSGSQNTLYF | 25 | 359 | CASSSTGDEQYF | 23 |
| 601 | CASSQGSQNTLYF | 28 | 738 | CASSLGQANTEVFF | 27 | 1176 | CASSRQGAGNTLYF | 25 | 1550 | CASSLQGGQNTLYF | 23 |
| 585 | CASSPNTGQLYF | 28 | 659 | CASGDARDTQYF | 27 | 1048 | CASSENTGQLYF | 25 | 360 | CSADSYEQYF | 23 |
| 576 | CASSPGGQNTLYF | 28 | 691 | CASSGQANTEVFF | 27 | 1177 | CASSRQGDTEVFF | 25 | 1661 | CAWSLGDQDTQYF | 23 |
| 38 | CASSRQQDTQYF | 28 | 783 | CASSPGTTNSDYTF | 27 | 1053 | CASSFNYAEQFF | 25 | 1663 | CAWSLGGQDTQYF | 23 |
| 618 | CASSRGQNTLYF | 28 | 790 | CASSQDSSQNTLYF | 27 | 1023 | CASGDRGSGNTLYF | 25 | 1662 | CAWSLGGNYAEQFF | 23 |
| 39 | CASSRDSQDTQYF | 28 | 765 | CASSLTGANTGQLYF | 27 | 1017 | CASGDAQSGNTLYF | 25 | 1518 | CASSLGAQNTLYF | 23 |
| 522 | CASSLGNQDTQYF | 28 | 680 | CASSDNQDTQYF | 27 | 1120 | CASSNTGQLYF | 25 | 1621 | CASSRDWGSYEQYF | 23 |
| 40 | CASSLQGYEQYF | 28 | 681 | CASSDNYAEQFF | 27 | 1159 | CASSQQGNTGQLYF | 25 | 1586 | CASSPGTDTEVFF | 23 |
| 41 | CASSDSSYEQYF | 28 | 828 | CGARDSAETLYF | 27 | 1074 | CASSLDRGEVFFF | 25 | 1593 | CASSPTGGAETLYF | 23 |
| 463 | CASSDSSQNTLYF | 28 | 710 | CASSLDRGAETLYF | 27 | 1208 | CAWSLGSQNTLYF | 25 | 1634 | CASSRQQNTLFY | 23 |
| 460 | CASSDSAETLYF | 28 | 660 | CASGDASAETLYF | 27 | 1202 | CASSSQANTEVFF | 25 | 1642 | CASSRTTSAETLYF | 23 |
| 462 | CASSDSSAETLYF | 28 | 793 | CASSQNTLYF | 27 | 250 | CASSLTGEDTQYF | 25 | 1639 | CASSRTGNTEVFF | 23 |
| 461 | CASSDSQNTLYF | 28 | 758 | CASSLQNTEVFF | 27 | 251 | CASSPGNTLYF | 25 | 1617 | CASSRDSSNERLFF | 23 |
| 632 | CASSSDSAETLYF | 28 | 794 | CASSQQGNTEVFF | 27 | 1186 | CASSRTNTEVFF | 25 | 1495 | CASSFNSAETLYF | 23 |
| 634 | CASSSGDQDTQYF | 28 | 744 | CASSLGTEVFF | 27 | 1179 | CASSRTANSDYTF | 25 | 1530 | CASSLGQGNTGQLYF | 23 |
| 547 | CASSLQGNSDYTF | 28 | 699 | CASSLAGSGNTLYF | 27 | 1129 | CASSPGQANTEVFF | 25 | 1536 | CASSLGSSGNTLYF | 23 |
| 512 | CASSLGDQDTQYF | 28 | 799 | CASSRDNSDYTF | 27 | 1122 | CASSPDNYAEQFF | 25 | 1649 | CASSSGTANSDYTF | 23 |
| 562 | CASSLTANSDYTF | 28 | 152 | CASSQYEQYF | 27 | 1020 | CASGDNSQNTLYF | 25 | 1626 | CASSRGQNTEVFF | 23 |
| 42 | CASSLGSSYEQYF | 28 | 944 | CASSPDWGQNTLYF | 26 | 252 | CASGDRDEQYF | 25 | 1537 | CASSLGTGGAETLYF | 23 |
| 440 | CASGAFNQAPLF | 28 | 153 | CASSRQGQDTQYF | 26 | 1011 | CASGDAGDTGQLYF | 25 | 1546 | CASSLQENTLYF | 23 |
| 450 | CASGDRDTQYF | 28 | 154 | CASSLQGAEQFF | 26 | 1026 | CASGDSNERLFF | 25 | 1519 | CASSLGASAETLYF | 23 |
| 43 | CASGDGDTQYF | 28 | 895 | CASSLEGANSDYTF | 26 | 1151 | CASSQEGSQNTLYF | 25 | 1503 | CASSGTNTEVFF | 23 |
| 455 | CASGDWGSQNTLYF | 28 | 954 | CASSPQGNTGQLYF | 26 | 1054 | CASSGQGNTEVFF | 25 | 1494 | CASSFGQNTEVFF | 23 |
| 446 | CASGDAGYEQYF | 28 | 155 | CASSDNYEQYF | 26 | 1059 | CASSGTGQNTLYF | 25 | 1656 | CASSTGTANTEVFF | 23 |
| 445 | CASGDAGSQNTLYF | 28 | 859 | CASSDRNTEVFF | 26 | 1190 | CASSSANTGQLYF | 25 | 361 | CASGEQYF | 23 |
| 44 | CASGDYEQYF | 28 | 156 | CASSDRGDTQYF | 26 | 253 | CASGDGEQYF | 25 | 1531 | CASSLGQGNYAEQFF | 23 |
| 627 | CASSRQNSDYTF | 28 | 860 | CASSDWDQDTQYF | 26 | 1034 | CASRDWGSAETLYF | 25 | 1499 | CASSFWGNYAEQFF | 23 |
| 577 | CASSPGHERLFF | 28 | 157 | CASSDAEQFF | 26 | 254 | CASSLDKYEQYF | 24 | 1612 | CASSQTGGQNTLYF | 23 |
| 45 | CASSLEDTQYF | 28 | 158 | CASSGQYEQYF | 26 | 1363 | CASSPGTENTLYF | 24 | 1605 | CASSQGANTEVFF | 23 |
| 480 | CASSLDERLFF | 28 | 874 | CASSGISNSDYTF | 26 | 255 | CASSRDNSYEQYF | 24 | 1596 | CASSPTTNTEVFF | 23 |
| 516 | CASSLGGAETLYF | 28 | 868 | CASSFSGAQDTQYF | 26 | 1336 | CASSLTNSDYTF | 24 | 362 | CASSPDWGYEQYF | 23 |
| 631 | CASSSAETLYF | 28 | 840 | CASGDNSGNTLYF | 26 | 1246 | CASSDNNQAPLF | 24 | 1615 | CASSRDNNQDTQYF | 23 |
| 46 | CASSLEGDEQYF | 28 | 846 | CASGDSYAEQFF | 26 | 1245 | CASSDNNERLFF | 24 | 1543 | CASSLLGTSAETLYF | 23 |
| 588 | CASSPSAETLYF | 28 | 831 | CASGDADSGNTLYF | 26 | 1241 | CASSDASQNTLYF | 24 | 1524 | CASSLGHYAEQFF | 23 |
| 611 | CASSRDSAETLYF | 28 | 835 | CASGDAGGNTLYF | 26 | 256 | CASSDAGGSYEQYF | 24 | 1534 | CASSLGQYNSPLYF | 23 |
| 557 | CASSLSGNTLYF | 28 | 159 | CASSSGGQDTQYF | 26 | 1243 | CASSDGDTGQLYF | 24 | 1653 | CASSSTGDTGQLYF | 23 |
| 548 | CASSLQGNTEVFF | 28 | 160 | CASSLGGAEQFF | 26 | 1272 | CASSGTGNYAEQFF | 24 | 363 | CASSLQGEDTQYF | 23 |
| 486 | CASSLDSAETLYF | 28 | 927 | CASSLTDSGNTLYF | 26 | 257 | CASSGTGDEQYF | 24 | 364 | CASSLAGGEQYF | 23 |
| 47 | CASSLGYEQYF | 28 | 161 | CASSPDAEQFF | 26 | 1226 | CASGDRNYAEQFF | 24 | 1533 | CASSLGQNQAPLF | 23 |
| 567 | CASSLTGSQNTLYF | 28 | 162 | CASSLGGSYEQYF | 26 | 1232 | CASGEGSQNTLYF | 24 | 1570 | CASSLTGYAEQFF | 23 |
| 487 | CASSLDSDYTF | 28 | 1004 | CASSWGNYAEQFF | 26 | 1229 | CASGDWDSAETLYF | 24 | 1525 | CASSLGLGENTLYF | 23 |
| 48 | CASSLGQYEQYF | 28 | 882 | CASSLDGNTLYF | 26 | 1220 | CASGDAPSQNTLYF | 24 | 1526 | CASSLGLSAETLYF | 23 |
| 534 | CASSLGSQNTLYF | 28 | 896 | CASSLEGQNTLYF | 26 | 1215 | CASGDADQDTQYF | 24 | 365 | CASSLGTGQDTQYF | 23 |
| 443 | CASGDAGNTLYF | 28 | 878 | CASSLAGNTEVFF | 26 | 1222 | CASGDGGNYAEQFF | 24 | 1454 | CASGDNSAETLYF | 23 |
| 459 | CASSDRGQNTLYF | 28 | 163 | CASSLAGDEQYF | 26 | 258 | CASSLVGAETLYF | 24 | 1457 | CASGDRDTEVFF | 23 |
| 630 | CASSRVGSDYTF | 28 | 164 | CASSLSGGYEQYF | 26 | 259 | CASSLGGEDTQYF | 24 | 1463 | CASGDWGGYAEQFF | 23 |
| 610 | CASSRDRDTEVFF | 28 | 889 | CASSLDSNQDTQYF | 26 | 1435 | CGARDWGSQNTLYF | 24 | 1467 | CASGETANTEVF | 23 |
| 560 | CASSLSQQNTLYF | 28 | 904 | CASSLGDTGQLYF | 26 | 1434 | CGARDWGNTGQLYF | 24 | 1466 | CASGESQNTLYF | 23 |
| 457 | CASRPGTANTGQLYF | 28 | 964 | CASSQGNYAEQFF | 26 | 1262 | CASSFRNTEVFF | 24 | 1442 | CASGDAGTANTEVFF | 23 |
| 564 | CASSLTDYNSPLYF | 28 | 165 | CASSYNQDTQYF | 26 | 1261 | CASSFRGSQNTLYF | 24 | 1437 | CASGDAGANTEVFF | 23 |
| 49 | CASSVDGSYEQYF | 28 | 994 | CASSSQGNTGQLYF | 26 | 1228 | CASGDVEQYF | 24 | 366 | CASSTGEDTQYF | 23 |
| 598 | CASSEASNSDYTF | 28 | 986 | CASSSDWGQDTQYF | 26 | 1239 | CASRWDNYEQYF | 24 | 1492 | CASSESAETLYF | 23 |
| 496 | CASSLDWGQDTQVF | 28 | 1003 | CASSWDSYAEQFF | 26 | 1286 | CASSLDSNERLFF | 24 | 367 | CASSPGTEDTQYF | 23 |
| 581 | CASSPGQNYAEQFF | 28 | 914 | CASSLQGGNTLYF | 26 | 1282 | CASSLDGSSAETLYF | 24 | 1557 | CASSLRDSGNTLYF | 23 |
| 50 | CASSPQDTQYF | 28 | 890 | CASSLDSQDTQYF | 26 | 1309 | CASSLGTSQNTLYF | 24 | 1508 | CASSLAGNSDYTF | 23 |
| 51 | CASSLDNYEQYF | 28 | 166 | CASSLEGEQYF | 26 | 260 | CASSLGDSDYTF | 24 | 1496 | CASSFQNTEVFF | 23 |
| 484 | CASSLDNYAEQFF | 28 | 167 | CASSRDRGYEQYF | 26 | 261 | CASSLVQDTQYF | 24 | 1504 | CASSHNQDTQYF | 23 |
| 488 | CASSLDSQNTLYF | 28 | 992 | CASSSQGNSDYTF | 26 | 1345 | CASSNSQNTLYF | 24 | 1490 | CASSDWGSQNTLYF | 23 |
| 604 | CASSQSLDNQDTQYF | 28 | 990 | CASSSGQQDTQYF | 26 | 1427 | CASSTGGQNTLYF | 24 | 1477 | CASSDAGNTEVFF | 23 |
| 648 | CASSYSAETLYF | 28 | 881 | CASSLDANTEVFF | 26 | 262 | CASSTQDTQYF | 24 | 1478 | CASSDASSGNTLYF | 23 |
| 559 | CASSLSQNTLYF | 28 | 888 | CASSLDSGNTLYF | 26 | 263 | CASSHSYEQYF | 24 | 368 | CASSDWGYEQYF | 23 |
| 544 | CASSLQGAETLYF | 28 | 168 | CASSLGGEQYF | 26 | 1266 | CASSGDSSGNTLYF | 24 | 1657 | CASSVNQDTQYF | 23 |
| 52 | CASSLEGYEQYF | 28 | 866 | CASSFPSGNTLYF | 26 | 1417 | CASSSQGSGNTLYF | 24 | 1453 | CASGDNQDTQYF | 23 |
| 53 | CASSLDEQYF | 28 | 169 | CASSLEEQYF | 26 | 264 | CASSSDRDEQYF | 24 | 1606 | CASSQGNTEVFF | 23 |
| 54 | CASSLEGNQDTQYF | 28 | 170 | CASGDDEQYF | 26 | 1420 | CASSSSAETLYF | 24 | 369 | CASSRDRDTQFY | 23 |
| 55 | CASSLEGDTQYF | 28 | 925 | CASSLSTGQLYF | 26 | 1413 | CASSSGQNTGQLYF | 24 | 370 | CASSQGYEQYF | 23 |
| 56 | CASSLDYEQYF | 28 | 171 | CASSRLPSYEQYF | 26 | 1318 | CASSLQGETLYF | 24 | 1491 | CASSEQGNTEVFF | 23 |
| 57 | CASSLGDTQYF | 28 | 977 | CASSRLGQDTQYF | 26 | 1281 | CASSLDGQNTLYF | 24 | 1476 | CASSDAGAETLYF | 23 |
| 519 | CASSLGGNTLYF | 28 | 172 | CASSLAGGQDTQYF | 26 | 1280 | CASSLDGAETLYF | 24 | 1474 | CASSANTGQLYF | 23 |
| 58 | CASSLGEQYF | 28 | 951 | CASSPGTTSAETLYF | 26 | 265 | CASSLEDSYEQYF | 24 | 1501 | CASSGQNTEVFF | 23 |
| 510 | CASSLGAETLYF | 28 | 906 | CASSLGQNTGQLYF | 26 | 1308 | CASSLGTNSDYTF | 24 | 1667 | CGARDRNTGQYLF | 23 |
| 492 | CASSLDSYAEQFF | 28 | 173 | CASSRTGGQDTQYF | 26 | 1341 | CASSLVGGAETLYF | 24 | 1668 | CGARDSQNTLYF | 23 |
| 518 | CASSLGGNTEVFF | 28 | 862 | CASSDWGQNTLYF | 26 | 1428 | CASSTGGYAEQFF | 24 | 1669 | CGARDWGSAETLYF | 23 |
| 59 | CASSQDTQYF | 28 | 871 | CASSGLGNYAEQFF | 26 | 1326 | CASSLRGNQDTQYF | 24 | 1636 | CASSRQYAEQFF | 23 |
| 597 | CASSQDWGQDTQYF | 28 | 872 | CASSGQGAETLYF | 26 | 266 | CASSLQGDTQYF | 24 | 371 | CASSDRYEQYF | 23 |
| 60 | CASSLDRYEQYF | 28 | 863 | CASSFGAETLYF | 26 | 1300 | CASSLGGANTEVFF | 24 | 372 | CASSYYEQYF | 23 |
| 61 | CASSGTGGYEQYF | 28 | 991 | CASSSGTNTEVFF | 26 | 1297 | CASSLGDYAEQFF | 24 | 1511 | CASSLDNQAPLF | 23 |
| 649 | CASSYSQNTLYF | 28 | 932 | CASSLTGGSQNTLYF | 26 | 1227 | CASGDTMSDUTF | 24 | 1560 | CASSLSANSDYTF | 23 |
| 641 | CASSSSAEFLYF | 28 | 886 | CASSLDQAPLF | 26 | 1344 | CASSNSGNTLYF | 24 | 1616 | CASSRDRYAEQFF | 23 |
| 633 | CASSSDWGNYAEFF | 28 | 899 | CASSLESAETLYF | 26 | 1373 | CASSPTGDQDTQYF | 24 | 1473 | CASSAGSQNTLYF | 23 |
| 62 | CASSSYEQYF | 28 | 174 | CASSLEGQDTQYF | 26 | 1389 | CASSRDSGNTLYF | 24 | 1647 | CASSSGGTEVFF | 23 |
| 635 | CASSSGQNTEVFF | 28 | 903 | CASSLGDSGNTLYF | 26 | 1350 | CASSPDWGSAETLYF | 24 | 1648 | CASSSGQNYAEQFF | 23 |
| 63 | CASSSGQYEQYF | 28 | 175 | CASSLVGDEQYF | 26 | 1314 | CASSLNSGNTLYF | 24 | 1521 | CASSLGDTLYF | 23 |
| 642 | CASSSSQNTLYF | 28 | 1001 | CASSTGNTGQLYF | 26 | 267 | CASSLPGQDTQYF | 24 | 1568 | CASSLTGGNYAEQFF | 23 |
| 646 | CASSWGQNTLYF | 28 | 981 | CASSRQGANTGQLYF | 26 | 1301 | CASSLGGTGQLYF | 24 | 1439 | CASGDAGGNTGQLYF | 23 |
| 540 | CASSLNSAETLYF | 28 | 176 | CASSPQGYEQYF | 26 | 1305 | CASSLGTENTLYF | 24 | 1548 | CASSLQGDTGQLYF | 23 |
| 568 | CASSLTSAETLYF | 28 | 943 | CASSPDSQNTLYF | 26 | 1371 | CASSPRDNYAEQFF | 24 | 1610 | CASSQQGTEVFF | 23 |
| 545 | CASSLQGAGNTLYF | 28 | 946 | CASSPGGNYAEQFF | 26 | 1393 | CASSRLGDQDTQYF | 24 | 1664 | CAWSLGSAETLYF | 23 |
| 550 | CASSLQGSAETLYF | 28 | 960 | CASSPTSAETLYF | 26 | 1394 | CASSRLGDYAEQFF | 24 | 1611 | CASSQTANSDYTF | 23 |
| 64 | CASSLLGGAEQFF | 28 | 945 | CASSPGAETLYF | 26 | 1381 | CASSRANTGQLYF | 24 | 1555 | CASSLRANTEVFF | 23 |
| 504 | CASSLEGNTLYF | 28 | 978 | CASSRNTGQLYF | 26 | 1401 | CASSRQNTLYF | 24 | 1535 | CASSRQSAETLYF | 23 |
| 65 | CASSLDRDEQYF | 28 | 973 | CASSRDRNTGQLYF | 26 | 1319 | CASSLQFFAEQFF | 24 | 1644 | CASSSDSSQNTLYF | 23 |
| 66 | CASSLDQDTQYF | 28 | 972 | CASSRDRGAEQFF | 26 | 1290 | CASSLEGDAEQFF | 24 | 1581 | CASSPGNTGQLYF | 23 |
| 505 | CASSLEGNYAEQFF | 28 | 941 | CASSLWGNYAEQFF | 26 | 268 | CASSLGQGYEQYF | 24 | 1625 | CASSRGNTEVFF | 23 |
| 67 | CASSLEGSSYEQYF | 28 | 861 | CASSDWGNYAEQFF | 26 | 1238 | CASRQGSQNTLYF | 24 | 1618 | CASSRDTNTEVFF | 23 |
| 507 | CASSLEGSQNTLYF | 28 | 853 | CASSASQNTLYF | 26 | 1429 | CASSTGNTEVFF | 24 | 1643 | CASSSANTEVFF | 23 |
| 475 | CASSLAGNYAEQFF | 28 | 870 | CASSGDSYAEQFF | 26 | 1397 | CASSRQGSAETLYF | 24 | 1600 | CASSQDNSGNTLYF | 23 |
| 476 | CASSLAGSQNTLYF | 28 | 997 | CASSSTASQNTLYF | 26 | 269 | CASSPTGNSDYTF | 24 | 1603 | CASSQDWGQNTLYF | 23 |
| 533 | CASSLGSAETLYF | 28 | 999 | CASSSTSAETLYF | 26 | 1347 | CASSPDSNQDTQYF | 24 | 1587 | CASSPNSAETLYF | 23 |
| 532 | CASSLGQTEVFF | 28 | 177 | CASSSDSYEQYF | 26 | 1359 | CASSPGLQDTQYF | 24 | 1554 | CASSLQGTNERLFF | 23 |
| 517 | CASSLGGNSDYTF | 28 | 1002 | CASSWDSQNTLYF | 26 | 1400 | CASSRQISNERLFF | 24 | 1528 | CASSLGQANTGQLYF | 23 |
| 68 | CASSLGGYEQYF | 28 | 851 | CASRDWGNYAEQFF | 26 | 270 | CASSRLGEDTQYF | 24 | 1552 | CASSLGGYAEQFF | 23 |
| 69 | CASSLGAEQFF | 28 | 852 | CASRGAETLYF | 26 | 1384 | CASSRDRAETLYF | 24 | 1517 | CASSLEQNTEVFF | 23 |
| 561 | CASSLSSQNTLYF | 28 | 958 | CASSPTGNTGQLYF | 26 | 271 | CASSRTFFAETLYF | 24 | 1514 | CASSLDWGDAEQFF | 23 |
| 70 | CASSLSGYEYF | 28 | 947 | CASSPGQYAEQFF | 26 | 1404 | CASSRSAETLYF | 24 | 1458 | CASGDRGNYAEQFF | 23 |
| 636 | CASSSGSQNTLYF | 28 | 979 | CASSRQANSDYTF | 26 | 272 | CASSLQGQDTQYF | 24 | 1459 | CASGDRNSDYTF | 23 |
| 638 | CASSSNTGQLYF | 28 | 980 | CASSRQGANTEVFF | 26 | 1335 | CASSLTISNERLFF | 24 | 1455 | CASGDQNTLYF | 23 |
| 71 | CASSSSYEQYF | 28 | 971 | CASSRDNSGNTLYF | 26 | 1421 | CASSSSSQNTLYF | 24 | 1465 | CASGEQDTQYF | 23 |
| 647 | CASSWGSAETLYF | 28 | 178 | CASSRTGEDTQYF | 26 | 1424 | CASSSTSQNTLYF | 24 | 1438 | CASGDAGGNTEVFF | 23 |
| 556 | CASSLSAETLYF | 28 | 179 | CASSRTGGYEQYF | 26 | 1423 | CASSSTGNTGQLYF | 24 | 1440 | CASGDAGGSQNTLYF | 23 |
| 493 | CASSLDTEVFF | 28 | 917 | CASSLQSQNTLYF | 26 | 1237 | CASRNTGQLYF | 24 | 1461 | CASGDVGSQNTLYF | 23 |
| 485 | CASSLDRYAEQFF | 28 | 873 | CASSGTANTGQLYF | 26 | 1402 | CASSRQSNTEVFF | 24 | 373 | CASSGQGYEQYF | 23 |
| 497 | CASSLDWGSAETLYF | 28 | 988 | CASSSGQANTEVFF | 26 | 1372 | CASSPTASAETLYF | 24 | 1598 | CASSQANTEVFF | 23 |
| 72 | CASSLGQDTQYF | 28 | 931 | CASSLTGGNTLYF | 26 | 273 | CASSPGQGYEQYF | 24 | 1483 | CASSDRANTEVFF | 23 |
| 73 | CASSLGGQDTQYF | 28 | 918 | CASSLRAETLYF | 26 | 1356 | CASSPGLGYAEQFF | 24 | 374 | CASSQEGDTQYF | 23 |
| 469 | CASSFSQNTLYF | 28 | 180 | CASSLQGSSYEQYF | 26 | 274 | CASSPGDTQYF | 24 | 375 | CASSQDWEDTQYF | 23 |
| 531 | CASSLGQSQNTLYF | 28 | 920 | CASSLRDTGQLYF | 26 | 275 | CASSRSSYEQYF | 24 | 376 | CASSDWGSYEQYF | 23 |
| 74 | CASSENQDTQYF | 28 | 883 | CASSLDIYAEQFF | 26 | 1317 | CASSLQGDTEVFF | 24 | 1608 | CASSQGTANSDYTF | 23 |
| 75 | CASGQDTQYF | 28 | 898 | CASSLENTGQLYF | 26 | 276 | CASSLSGDEQYF | 24 | 1629 | CASSRLGSSAETLYF | 23 |
| 609 | CASSRDNYAEQFF | 28 | 939 | CASSINNQDTQYF | 26 | 1322 | CASSLQGYNSPLYF | 24 | 1471 | CASRDSSAETLYF | 23 |
| 572 | CASSPDSSQNTLYF | 28 | 181 | CASSLTGNTEVFF | 26 | 277 | CASSSGYEQYF | 24 | 1588 | CASSPNSQNTLYF | 23 |
| 643 | CASSTSAETLYF | 28 | 182 | CASSLGGDTQYF | 26 | 1291 | CASSLEGGYAEQFF | 24 | 1451 | CASGDGTTNTEVFF | 23 |
| 574 | CASSPDWGNYAEQFF | 28 | 865 | CASSFGSAETLYF | 26 | 1279 | CASSLANTEVFF | 24 | 1670 | CSADTEVFF | 23 |
| 595 | CASSPYAEQFF | 28 | 982 | CASSRQNTGQLYF | 26 | 1432 | CASSWDRNTEVFF | 24 | 1532 | CASSLGQGQNTLYF | 23 |
| 573 | CASSPDWGENTLYF | 28 | 985 | CASSSDSQNTLYF | 26 | 278 | CASSQTGGQDTQYF | 24 | 1449 | CASGDGGNQDTQYF | 23 |
| 76 | CASSRDWGYEQYF | 28 | 183 | CASSLDWGYEQYF | 26 | 1380 | CASSQSQNTLYF | 24 | 1441 | CASGDAGSGNTLYF | 23 |
| 77 | CASSLDSYEQYF | 28 | 887 | CASSLDRNTLYF | 26 | 1349 | CASSPDWGAETLYF | 24 | 377 | CASGDVDTQYF | 23 |
| 555 | CASSLRGSQNTLYF | 28 | 877 | CASSLAGNQDTQYF | 26 | 1362 | CASSPGQSAETLYF | 24 | 1500 | CASSGDWGNYAEQFF | 23 |
| 542 | CASSLQANSDYTF | 28 | 967 | CASSQGTANTGQLYF | 26 | 1374 | CASSPTSQNTLYF | 24 | 1485 | CASSDRNERLFF | 23 |
| 491 | CASSLDSSQNTLYF | 28 | 966 | CASSGSGNTLYF | 26 | 1385 | CASSRDRGAETLYF | 24 | 1446 | CASGDASSGNTLYF | 23 |
| 537 | CASSLGTTNSDYF | 28 | 959 | CASSPTGNYAEQFF | 26 | 1339 | CASSLVAETLYF | 24 | 1911 | CAWSLGSSAETLYF | 22 |
| 529 | CASSLGQNYAEQFF | 28 | 952 | CASSPQANTGQLYF | 26 | 1328 | CASSLRGNTLYF | 24 | 1869 | CASSRGDQDTQYF | 22 |
| 535 | CASSLGSSAETLYF | 28 | 961 | CASSPTSSQNTLYF | 26 | 279 | CASSLTGYEQYF | 24 | 378 | CASSLGQGDTQYF | 22 |
| 575 | CASSPGDQDTQYF | 28 | 942 | CASSPDNSGNTLYF | 26 | 280 | CASSLDIYEQYF | 24 | 1716 | CASSDRETLYF | 22 |
| 617 | CASSRDWGQDTQYF | 28 | 184 | CASSPGQSSYEQYF | 26 | 1418 | CASSSQGSQNTLYF | 24 | 1718 | CASSDRGSAETLYF | 22 |
| 490 | CASSLDSSGNTLYF | 28 | 953 | CASSPQGAGNTLYF | 26 | 1412 | CASSSDAEQFF | 24 | 1717 | CASSDRGQDTQYF | 22 |
| 620 | CASSRQANTEVFF | 28 | 919 | CASSLRDNQDTQYF | 26 | 281 | CASSLSQDTQYF | 24 | 379 | CASSDDEQYF | 22 |
| 78 | CASSRQYEQYF | 28 | 185 | CASSYSYEQYF | 26 | 1330 | CASSLRGSAETLYF | 24 | 1691 | CASGDRGQDTQYF | 22 |
| 79 | CASSLKDTQYT | 28 | 996 | CASSSTANTEVFF | 26 | 1324 | CASSLRGAETLYF | 24 | 1682 | CASGDGGSQNTLYF | 22 |
| 558 | CASSLSGSNYAEQFF | 28 | 936 | CASSLTNTEVFF | 26 | 282 | CASSLAGSSYEQYF | 24 | 1687 | CASGDNSDYTF | 22 |
| 587 | CASSPQGNTEVFF | 28 | 934 | CASSLTGQNTLYF | 26 | 283 | CASSLGGREQYF | 24 | 1778 | CASSLGLNQDTQYF | 22 |
| 80 | CASSLGQNTEVFF | 28 | 935 | CASSLTGSAETLYF | 26 | 1298 | CASSLGENTEVFF | 24 | 1832 | CASSPQGNYAEQFF | 22 |
| 81 | CASSFGTGDEQYF | 28 | 897 | CASSLEGSAETLYF | 26 | 1258 | CASSFGGSQNTLYF | 24 | 1803 | CASSLRGYAEQFF | 22 |
| 552 | CASSLGSQNTLYF | 28 | 900 | CASSLGASQNTLYF | 26 | 1351 | CASSPDWGYAEQFF | 24 | 1895 | CASSSQNYAEQFF | 22 |
| 494 | CASSLDTGQLYF | 28 | 938 | CASSLVGNYAEQFF | 26 | 1361 | CASSPGQNERLFF | 24 | 1887 | CASSSGSSAETLYF | 22 |
| 481 | CASSLDGSQNTLYF | 28 | 186 | CASSINEQYF | 26 | 1358 | CASSPGLNYAEQFF | 24 | 1696 | CASGETGNYAEQFF | 22 |
| 508 | CASSLEGYAEQFF | 28 | 926 | CASSLIASQNTLYF | 26 | 1354 | CASSPGLGAETLYF | 24 | 1918 | CGARTVSNERLFF | 22 |
| 530 | CASSLGQQNTLYF | 28 | 930 | CASSLTGDQDTQYF | 26 | 1406 | CASSRTENTLYF | 24 | 1914 | CGARDSSQNTLYF | 22 |
| 526 | CASSLGNTLYT | 28 | 187 | CASSLDRGNTEVFF | 26 | 1255 | CASSEGAETLYF | 24 | 1915 | CGARDSSYNSPLYF | 22 |
| 513 | CASSLGENTLYT | 28 | 188 | CASSLLGYEQYF | 26 | 1249 | CASSDRGSGNTLYF | 24 | 1916 | CGARDWGSSYEQYF | 22 |
| 82 | CASSLGEDTQYF | 28 | 189 | CASSQSSYEQYF | 26 | 1247 | CASSDQNTLYF | 24 | 1735 | CASSGTDYAEQFF | 22 |
| 464 | CASSFDSQNTLYF | 28 | 976 | CASSRDWGNTGQLYF | 26 | 1251 | CASSDSGNTLYF | 24 | 1819 | CASSPANTGQLYF | 22 |
| 466 | CASSFGSQNTLYF | 28 | 190 | CASSEGDTQYF | 26 | 1242 | CASSDDNSGNTLYF | 24 | 1784 | CASSLGSQDTQYF | 22 |
| 626 | CASSRQGSQNTLYF | 28 | 191 | CASSDRDTQYF | 26 | 1269 | CASSGTAETLYF | 24 | 1880 | CASSSGGSAETLYF | 22 |
| 83 | CASSRQNQDTQYF | 28 | 855 | CASSDAGSQNTLYF | 26 | 284 | CASSGTGDTQYF | 24 | 1794 | CASSLNAETLYF | 22 |
| 605 | CASSRDIQDTQYF | 28 | 869 | CASSGDSQNTLYF | 26 | 1270 | CASSGTGGAETLYF | 24 | 380 | CASSLTGGSYEQYF | 22 |
| 84 | CASSPSSYEYF | 28 | 850 | CASGGANTEVFF | 26 | 1264 | CASSGDNYAEQFF | 24 | 1763 | CASSLEGASQNTLYF | 22 |
| 590 | CASSPSSAETLYT | 28 | 843 | CASGDRYEYF | 26 | 1235 | CASGTTNTEVFF | 24 | 1751 | CASSLDGSAETLYF | 22 |
| 586 | CASSPNYAEQFF | 28 | 842 | CASGDRGNTEVFF | 26 | 1225 | CASGDRGAETLYF | 24 | 1788 | CASSLGTNQDTQYF | 22 |
| 580 | CASSPGQNTEVFF | 28 | 845 | CASGDSSGNTLYF | 26 | 1230 | CASGDWGNTGQLFY | 24 | 381 | CASSLGSDYTF | 22 |
| 85 | CASSPDSYEQYF | 28 | 832 | CASGDAETLYF | 26 | 1231 | CASGDWGQNTLYF | 24 | 1818 | CASSLVQNTLYF | 22 |
| 607 | CASSRDNNERLFF | 28 | 983 | CASSRTDSGNTLYF | 26 | 1219 | CASGDANTEVFF | 24 | 1801 | CASSLRANTGQLYF | 22 |
| 86 | CASSRDNYEQYF | 28 | 950 | CASSPGTTNTEVFF | 26 | 1343 | CASSLWGSAETLYF | 24 | 1848 | CASSQEGNTEVFF | 22 |
| 614 | CASSRDSYAEQFF | 28 | 913 | CASSLLGDQDTQYF | 26 | 285 | CASSGTEDTQYF | 24 | 382 | CASSQGAEQFF | 22 |
| 87 | CASSRDSYEQYF | 28 | 891 | CASSLDTSQNTLYF | 26 | 1267 | CASSGQGNSDYTF | 24 | 1867 | CASSRDWGAETLYF | 22 |
| 616 | CASSRDWGNYAEQFP | 28 | 854 | CASSDAGNTLYF | 26 | 1366 | CASSPLGNYAEQFF | 24 | 1739 | CASSGTSSAETLYF | 22 |
| 88 | CASSLDRVEQYF | 28 | 192 | CASSDGDTQYF | 26 | 1387 | CASSRDRGQNTLYF | 24 | 1896 | CASSSTGSQNTLYF | 22 |
| 569 | CASSLWGSQNTLYF | 28 | 867 | CASSFQGNTEVFF | 26 | 286 | CASSLTYEQYF | 24 | 1888 | CASSSGTGAETLYF | 22 |
| 644 | CASSWDNYAEQFF | 28 | 193 | CASSFYEQYF | 26 | 287 | CASSLDDTQYF | 24 | 383 | CASSSGDTQYF | 22 |
| 602 | CASSQNTGQLYF | 28 | 849 | CASGESAETLYF | 26 | 1303 | CASSLGNQAPLF | 24 | 1893 | CASSSQGNQDTQYF | 22 |
| 473 | CASSHSQNTLYF | 28 | 838 | CASGDASQNTLYF | 26 | 288 | CASSLGTEDTQYF | 24 | 1879 | CASSSGGNYAEQFF | 22 |
| 571 | CASSPDSSGNTLYF | 28 | 830 | CASGAETLYF | 26 | 1398 | CASSRQGSGNTLYF | 24 | 1800 | CASSLQYAEQFF | 22 |
| 623 | CASSRQGNTEVFF | 28 | 1006 | CSSSQGTNERLFF | 26 | 1415 | CASSSNSGNTLYF | 24 | 384 | CASSLDRGAEQFF | 22 |
| 621 | CASSRQGANSDYTF | 28 | 993 | CASSSQGNTEVFF | 26 | 1327 | CASSLRGNTGQLYF | 24 | 1770 | CASSLEQNTGQLYF | 22 |
| 541 | CASSLNTGQLYF | 28 | 910 | CASSLGTSAETLYF | 26 | 1285 | CASSLDRNYAEQFF | 24 | 1764 | CASSLEGGAETLYF | 22 |
| 551 | CASSLQGSGNTLYF | 28 | 902 | CASSLGDSAETLYF | 26 | 1216 | CASGDAEQYF | 24 | 1746 | CASSLAQNTLYF | 22 |
| 89 | CASSLYAEQFF | 28 | 901 | CASSLGDNYAEQFF | 26 | 1365 | CASSPGTSQNTLYF | 24 | 1890 | CASSSNSAETLYF | 22 |
| 90 | CASSLDAEQFF | 28 | 194 | CASSYEQYF | 26 | 1288 | CASSLDSYNSPLYF | 24 | 385 | CASSSGGYEQYF | 22 |
| 91 | CASSDSYEQYF | 28 | 933 | CASSLTGNTLYF | 26 | 1340 | CASSLVGDQGTQYF | 24 | 386 | CASSWDNYEQYF | 22 |
| 524 | CASSLGNTEVFF | 28 | 916 | CASSLQISNERLFF | 26 | 1259 | CASSFNTGQLYF | 24 | 1771 | CASSLEQYAEQFF | 22 |
| 515 | CASSLGETLYF | 28 | 928 | CASSLTENTLYF | 26 | 1292 | CASSLEGQQDTQYF | 24 | 1729 | CASSFRTEMTEVFF | 22 |
| 467 | CASSFANTEVFF | 28 | 864 | CASSFGGAETLYF | 26 | 1313 | CASSLNNQDTQYF | 24 | 387 | CASSRDWGQNTLYF | 22 |
| 92 | CASSFGTEVFF | 28 | 848 | CASGEGQNTLYF | 26 | 1302 | CASSLGLYAEQFF | 24 | 1868 | CASSRTGQDTQYF | 22 |
| 563 | CASSLTANTEVFF | 28 | 984 | CASSRTSQNTLYF | 26 | 1310 | CASSLLGDYAEQFF | 24 | 388 | CASSLDRGQNTLYF | 22 |
| 527 | CASSLGQNSDYTF | 28 | 892 | CASSLDWGNTLYF | 26 | 1254 | CASSDYAEQFF | 24 | 1756 | CASSLEGAQDTQYF | 22 |
| 465 | CASSFGQNTLYF | 28 | 1005 | CSKDSAETLYF | 26 | 1332 | CASSLSGDQDTQYF | 24 | 1762 | CASSLEGAQDTQYF | 22 |
| 500 | CASSLEGAETLYF | 28 | 195 | CASSRQSSYEQYF | 26 | 1436 | CSADSQNTLYF | 24 | 1780 | CASSLGLSQNTLYF | 22 |
| 612 | CASSRDSQNTLYF | 28 | 949 | CASSPGTSSAETLYF | 26 | 289 | CSAEDTQYF | 24 | 1907 | CASSYRNTEVFF | 22 |
| 546 | CASSLQGANTEVFF | 28 | 885 | CASSLDNINQDTQYF | 26 | 1425 | CASSTGDQDTQYF | 24 | 1892 | CASSSNSQNTLYF | 22 |
| 603 | CASSQQGSQNTLYF | 28 | 911 | CASSLGTTNTEVFF | 26 | 1411 | CASSRTSAETLYF | 24 | 1894 | CASSSQNTEVFF | 22 |
| 589 | CASSPSQNTLYF | 28 | 915 | CASSLQGNQDTQYF | 26 | 1433 | CGARDHTSNTEVFF | 24 | 389 | CASSLTGQDTQYF | 22 |
| 592 | CASSPTASQNTLYF | 28 | 876 | CASSLAGDTGQLYF | 26 | 1378 | CASSQEGAETLYF | 24 | 1786 | CASSLGTDYAEQFF | 22 |
| 593 | CASSPTGAETLYF | 28 | 921 | CASSLRGNTEVFF | 26 | 1348 | CASSPDSYAEQFF | 24 | 1783 | CASSLQNERLFF | 22 |
| 93 | CASSPDNYEQYF | 28 | 908 | CASSLGSGNTLYF | 26 | 1369 | CASSPQNTLYF | 24 | 1706 | CASRQNTEVFF | 22 |
| 94 | CASSPGQYEQYF | 28 | 989 | CASSSGQNTLYF | 26 | 1386 | CASSRDRGNSDYTF | 24 | 1862 | CASSRDRGTEVFF | 22 |
| 579 | CASSPGQGYAEQFF | 28 | 875 | CASSLAANTGQLYF | 26 | 290 | CASSRDIYEQYF | 24 | 1824 | CASSPGLGSAETLYF | 22 |
| 624 | CASSRQGNTGQLYF | 28 | 965 | CASSQGSAETLYF | 26 | 1296 | CASSLEQTEVFF | 24 | 390 | CASSPGGYEQYF | 22 |
| 629 | CASSRSQNTLYF | 28 | 884 | CASSLDNERLFF | 26 | 1283 | CASSLDKNTLYF | 24 | 1840 | CASSPTNSGNTLYF | 22 |
| 95 | CASSLQDTQYF | 28 | 963 | CASSQDWGSQNTLYF | 26 | 1376 | CASSQDNYAEQFF | 24 | 391 | CASSPGQGDTQYF | 22 |
| 96 | CASSLRDTQYF | 28 | 955 | CASSPQGSQNTLYF | 26 | 1388 | CASSRDRGYAEQFF | 24 | 1831 | CASSPQGDTGQLYF | 22 |
| 549 | CASSLQGNTGQLYF | 28 | 975 | CASSRDTGQLYF | 26 | 291 | CASSLRAEQFF | 24 | 1836 | CASSPSGNTLYF | 22 |
| 538 | CASSLLGNYAEQFF | 28 | 929 | CASSLTGANTEVFF | 26 | 292 | CASSLLGEDTQYF | 24 | 392 | CASSLPGGYEQYF | 22 |
| 502 | CASSLEGNTEVFF | 28 | 922 | CASSLRGSGNTLYF | 26 | 1273 | CASSGTTNTEVFF | 24 | 1802 | CASSLRDTLYF | 22 |
| 483 | CASSLDNSGNTLYF | 28 | 907 | CASSLGQSAETLYF | 26 | 1379 | CASSQRDWCYAEQFF | 24 | 393 | CASSAQDTQYF | 22 |
| 498 | CASSLEANSDYTF | 28 | 912 | CASSLGVSNERLFF | 26 | 293 | CASSFGAEQFF | 24 | 1891 | CASSSNSDYTF | 22 |
| 503 | CASSLEGNTGQLYF | 28 | 879 | CASSLAGNTGQLYF | 26 | 1353 | CASSPGANTEVFF | 24 | 1702 | CASRDNANTEVFF | 22 |
| 521 | CASSLGGSQNTLYF | 28 | 924 | CASSLSNERLFF | 26 | 1409 | CASSRTGGSQNTLYF | 24 | 1866 | CASSRDWEDTQYF | 22 |
| 523 | CASSLGNSGNTLYF | 28 | 841 | CASGDPGNYAEQFF | 26 | 1295 | CASSLENYAEQFF | 24 | 1835 | CASSPRLGQDTQYF | 22 |
| 97 | CASSLGDEYF | 28 | 834 | CASGDAGEQYF | 26 | 1383 | CASSRDNNNQAPLF | 24 | 394 | CASSPGGGYEQYF | 22 |
| 98 | CASSLVAEQFF | 28 | 196 | CASSRDREDTQYF | 26 | 1352 | CASSPDYAEQFF | 24 | 1876 | CASSRTGGNTEVFF | 22 |
| 99 | CASSFSYEQYF | 28 | 905 | CASSLGGSNERLFF | 26 | 1338 | CASSLTVSNERLFF | 24 | 1721 | CASSEGAGNTLYF | 22 |
| 565 | CASSLTGGYAEQFF | 28 | 937 | CASSLTSSAETLYF | 26 | 1312 | CASSLLGSQNTLYF | 24 | 1886 | CASSSGQYAEQFF | 22 |
| 495 | CASSLDWGNYAEQFF | 28 | 894 | CASSLEDTGQLYF | 26 | 1315 | CASSLQANTGQLYF | 24 | 1798 | CASSLQGERLFF | 22 |
| 582 | CASSPGQSQNTLYF | 28 | 844 | CASGDSQNTLYF | 26 | 1236 | CASRDRNYAEQFF | 24 | 1760 | CASSLDWGAETLYF | 22 |
| 628 | CASSRQNTEVFF | 28 | 968 | CASSQNTEVFF | 26 | 1337 | CASSLTSSQNTLYF | 24 | 395 | CASSLTGSSYEQYF | 22 |
| 622 | CASSRQGNSDYTF | 28 | 857 | CASSDRAETLYF | 26 | 1430 | CASSTGSQNTLYF | 24 | 1753 | CASSLDNNERLFF | 22 |
| 625 | CASSRQGNYAEQFF | 28 | 836 | CASGDAGNSDYTF | 26 | 1426 | CASSTGGAETLYF | 24 | 1781 | CASSLGPNTEVFF | 22 |
| 613 | CASSRDSSAETLYF | 28 | 839 | CASGDAYAEQFF | 26 | 294 | CASSIQDTAYF | 24 | 396 | CASSLDRDTQYF | 22 |
| 100 | CASSLSYEQYF | 28 | 969 | CASSQQGANTEVFF | 26 | 295 | CASSIQYEQYF | 24 | 1855 | CASSQQGAGNTLYF | 22 |
| 101 | CASSDQDTQYF | 28 | 995 | CASSSSGNTLYF | 26 | 1275 | CASSIRDNYAEQFF | 24 | 1849 | CASSQEGQQDTQYF | 22 |
| 102 | CASSDAGDTQYF | 28 | 856 | CASSDASAETLYF | 26 | 1274 | CASSIGQNTEVFF | 24 | 1823 | CASSPGHQDTQYF | 22 |
| 458 | CASSAETLYF | 28 | 923 | CASSLSGSAETLYF | 26 | 1364 | CASSPGTGAETLYF | 24 | 1795 | CASSLNNERLFF | 22 |
| 470 | CASSGTANSDYTF | 28 | 1000 | CASSSYAEQFF | 26 | 1355 | CASSPGLGENTLYF | 24 | 1757 | CASSLDRGYAEQFF | 22 |
| 637 | CASSSGTANTEVFF | 28 | 974 | CASSRDSSGNTLYF | 26 | 1392 | CASSRGQNYAEQFF | 24 | 397 | CASSLGGGEQYF | 22 |
| 103 | CASGDSYEQYF | 28 | 987 | CASSSGNTEVFF | 26 | 1331 | CASSLSETLYF | 24 | 1908 | CASSYWGNYAEQFF | 22 |
| 453 | CASGDSSQNTLYF | 28 | 970 | CASSRDNSAETLYF | 26 | 1321 | CASSLQGTGQLYF | 24 | 1905 | CASSYNNQAPLF | 22 |
| 451 | CASGDSAETLYF | 28 | 833 | CASGDAGDQDTQYF | 26 | 1277 | CASSLAGANTEVFF | 24 | 1904 | CASSYGGQNTLYF | 22 |
| 454 | CASGDWGSAETLYF | 28 | 197 | CASSRYEQYF | 26 | 1299 | CASSLGEVFF | 24 | 1878 | CASSSGENTLYF | 22 |
| 449 | CASGDQDTQYF | 28 | 909 | CASSLGTGQLYF | 26 | 1304 | CASSLGQGNSDYTF | 24 | 398 | CASSSDRYEQYF | 22 |
| 444 | CASGDAGQNTLYF | 28 | 940 | CASSLVSQNTLYF | 26 | 1257 | CASSFDWGNYAEQFF | 24 | 1901 | CASSWGGQNTLYF | 22 |
| 441 | CASGDAEQFF | 28 | 957 | CASSPTGGNYAEQFF | 26 | 1263 | CASSFSGNTLYF | 24 | 1752 | CASSLDKEVFF | 22 |
| 447 | CASGDGSQNTLYF | 28 | 880 | CASSLAGSAETLYF | 26 | 1224 | CASGDRDQDTQYF | 24 | 399 | CASSLDSEQYF | 22 |
| 499 | CASSLEDSGNTLYF | 28 | 198 | CASSPQGTEVFF | 26 | 1223 | CASGDRANTEVFF | 24 | 400 | CASSLAGDTQYF | 22 |
| 482 | CASSLDNQDTQYF | 28 | 998 | CASSSTGNTEVFF | 26 | 1218 | CASGDAGSSYEQYF | 24 | 1774 | CASSLGGRAETLYF | 22 |
| 477 | CASSLANSDYTF | 28 | 948 | CASSPGQYNSPLYF | 26 | 1217 | CASGDAGGNYAEQFF | 24 | 1815 | CASSLVANSDYTF | 22 |
| 536 | CASSLGTANSDYTF | 28 | 893 | CASSLEANTGQLYF | 26 | 1234 | CASGGTANSDYTF | 24 | 401 | CASSLVGAEQFF | 22 |
| 104 | CASSLDTQYF | 28 | 847 | CASGDYAEQFF | 26 | 1391 | CASSRDWGNQDTQYF | 24 | 1777 | CASSLGLGQNTLYF | 22 |
| 105 | CASSFYAEQFF | 28 | 837 | CASGDANSGNTLYF | 26 | 1316 | CASSLQGDSDYTF | 24 | 1861 | CASSRDRGNTEVFF | 22 |
| 606 | CASSRDIYAEQFF | 28 | 962 | CASSQDSQNTLYF | 26 | 1407 | CASSRTGAETLYF | 24 | 1856 | CASSQTGNTGQLYF | 22 |
| 479 | CASSLASAETLYF | 28 | 858 | CASSDRDTEVFF | 26 | 1357 | CASSPGLNTGQLYF | 24 | 402 | CASSQDAEQFF | 22 |
| 448 | CASGDNYAEQFF | 28 | 956 | CASSPTANTGQLYF | 26 | 1325 | CASSLRGDTEVFF | 24 | 1829 | CASSPGTGNSDYTF | 22 |
| 106 | CASGDEQYF | 28 | 199 | CASSPGQGQDTQYF | 26 | 1329 | CASSLRGNYAEQFF | 24 | 403 | CASSPTGQDTQYF | 22 |
| 107 | CASGDAYEQYF | 28 | 1104 | CASSLQQDTQYF | 25 | 1306 | CASSLGTGAETLYF | 24 | 1860 | CASSRDNANSDYTF | 22 |
| 108 | CASSLYEQYF | 28 | 1157 | CASSQNSGNTLYF | 25 | 1395 | CASSRQDQDTQYF | 24 | 1864 | CASSRDSTNTEVFF | 22 |
| 599 | CASSQGLGDTLYF | 28 | 1127 | CASSPGGSQNTLYF | 25 | 1382 | CASSRDISAETLYF | 24 | 1809 | CASSLSTNSDYTF | 22 |
| 501 | CASSLEGNSDYTF | 28 | 1047 | CASSENSGNTLYF | 25 | 1422 | CASSSTANTGQLYF | 24 | 1807 | CASSLSGNSDYTF | 22 |
| 474 | CASSLAANSDYTF | 28 | 1049 | CASSETANTEVFF | 25 | 1431 | CASSTNTGQLYF | 24 | 404 | CASSLSGGSYEQYF | 22 |
| 553 | CASSLGTEVFF | 28 | 1046 | CASSEGSQNTLYF | 25 | 1375 | CASSQANSDYTF | 24 | 405 | CASSDGYEQF | 22 |
| 478 | CASSLANTGQLYF | 28 | 1044 | CASSDRNSDYTF | 25 | 1346 | CASSPDRGQNTLYF | 24 | 1710 | CASSDAGSAETLYF | 22 |
| 554 | CASSLQNTLYF | 28 | 1036 | CASSDAGGQNTLYF | 25 | 296 | CASSEGYQEQYF | 24 | 1711 | CASSGDDNTEVFF | 22 |
| 566 | CASSLTGNYAEQFF | 28 | 1038 | CASSDGAETLYF | 25 | 1252 | CASSDSNERLFF | 24 | 1740 | CASSGTTSAETLYF | 22 |
| 514 | CASSLGERLFF | 28 | 200 | CASSGDSYEQYF | 25 | 297 | CASSDRGYEQYF | 24 | 1432 | CASSGGAETLYF | 22 |
| 650 | CAWSLQGYNSPLYF | 28 | 1057 | CASSGSQNTLYF | 25 | 1253 | CASSDWGNQDTQYF | 24 | 1726 | CASSFNERLFF | 22 |
| 506 | CASSLEGSGNTLYF | 28 | 1195 | CASSSGNQDTQYF | 25 | 1233 | CASGETDSGNTLYF | 24 | 1723 | CASSFDRNTEVFF | 22 |
| 583 | CASSPGSQNTLYF | 28 | 1207 | CASSWGNQDTQYF | 25 | 298 | CASGEGEQYF | 24 | 1724 | CASSFGERLFF | 22 |
| 591 | CASSPTANSDYTF | 28 | 1028 | CASGGQGNTEVFF | 25 | 1271 | CASSGTGGNYAEQFF | 24 | 1697 | CASGETLYF | 22 |
| 578 | CASSPGQGNSDYTF | 28 | 1018 | CASGDDQDTQYF | 25 | 1408 | CASSRTGGNYAEQFF | 24 | 1692 | CASGDSANTEVFF | 22 |
| 511 | CASSLGANSDYTF | 28 | 1024 | CASGDRGSQNTLYF | 25 | 1396 | CASSRQGDTGQLYF | 24 | 1680 | CASGDDRGQNTLYF | 22 |
| 539 | CASSLNQDTQYF | 28 | 1008 | CASGDADTGQLYF | 25 | 299 | CASSDYEQYF | 24 | 1694 | CASGEEDTQYF | 22 |
| 543 | CASSL ANTEVFF | 28 | 1010 | CASGDAGANSDYTF | 25 | 1240 | CASSDANSGNTLYF | 24 | 1672 | CASGDAGEDTQYF | 22 |
| 471 | CASSGTTNSDYTF | 28 | 1016 | CASGDAGQDTQYF | 25 | 300 | CASSDAYEQYF | 24 | 1676 | CASGDAGQNSDYTF | 22 |
| 645 | CASSWDSAETI,YF | 28 | 1137 | CASSPGTAETLYF | 25 | 1244 | CASSDGGQNTLYF | 24 | 1673 | CASGDAGGEQYF | 22 |
| 525 | CASSLGNTGQINF | 28 | 1100 | CASSLPSAETLYF | 25 | 301 | CASGEYEQYF | 24 | 406 | CASSPGLGYEQYF | 22 |
| 615 | CASSRDWEQNTLYIF | 28 | 1113 | CASSLTASAETLYF | 25 | 1416 | CASSSNTEVFF | 24 | 1821 | CASSPDTSQNTLYF | 22 |
| 596 | CASSQDTEVFF | 28 | 1073 | CASSLDRDAEQFF | 25 | 1260 | CASSFRAETLYF | 24 | 407 | CASSRQGEDTQYF | 22 |
| 109 | CASSRDSSYEQYF | 28 | 1103 | CASSLQGSDYTF | 25 | 1333 | CASSLSGNQDTQYF | 24 | 408 | CASSLLGSSYEQYF | 22 |
| 110 | CASSIRDTQYF | 28 | 201 | CASGDFYEQYF | 25 | 1368 | CASSPQGSGNTLYF | 24 | 1785 | CASSLGTAETLYF | 22 |
| 584 | CASSPGTANTEVFF | 28 | 1022 | CASGDPSQNTLYF | 25 | 1311 | CASSLLGNTLYF | 24 | 1769 | CASSLEQGATELYF | 22 |
| 452 | CASGDSGNTLYF | 28 | 1211 | CGARQGYAEQFF | 25 | 1323 | CASSLQNYAEQFF | 24 | 1747 | CASSLASSGNTLYF | 22 |
| 442 | CASGDAGGQDTQYF | 28 | 1210 | CGARDWGYAEQFF | 25 | 1320 | CASSLQGSNERLFF | 24 | 409 | CASSLGTQDTQYF | 22 |
| 619 | CASSRGSAETLYF | 28 | 1209 | CGARDSNYAEQFF | 25 | 302 | CASSLGNTEVFF | 24 | 410 | CASSLSGGYAEQFF | 22 |
| 489 | CASSLDSSAETLYF | 28 | 202 | CASSQGTQYF | 25 | 303 | CASSLVGEQYF | 24 | 1850 | CASSQGNSGNTLYF | 22 |
| 600 | CASSQGQNTEVFF | 28 | 1189 | CASSSAANTEVFF | 25 | 1256 | CASSFAETLYF | 24 | 1837 | CASSPTGNTEVFF | 22 |
| 456 | CASRDSAETLYF | 28 | 203 | CASSHQDTQYF | 25 | 1276 | CASSISAETLYF | 24 | 1822 | CASSPGDTEVFF | 22 |
| 111 | CASSRTGYEQYF | 27 | 1172 | CASSRGNYAEQFF | 25 | 1360 | CASSPGNYAEQFF | 24 | 1808 | CASSLSNDYTF | 22 |
| 112 | CASSDAGYEQYF | 27 | 1067 | CASSLDGGQNTLYF | 25 | 1405 | CASSRTANTEVFF | 24 | 1897 | CASSSTNSDYTF | 22 |
| 113 | CASSQQDTQYF | 27 | 1106 | CASSLRSAETLYF | 25 | 1390 | CASSRDTYAEQFF | 24 | 411 | CASSLQGSYEQYF | 22 |
| 684 | CASSESQNTLYF | 27 | 1164 | CASSRDNTEVFF | 25 | 1342 | CASSLVGGQNTLYF | 24 | 1766 | CASSLEGNSGNTLYF | 22 |
| 683 | CASSDWGSAETLYF | 27 | 204 | CASSHYEQYF | 25 | 1399 | CASSRQGTEVFF | 24 | 1767 | CASSLEGRQNTLYF | 22 |
| 114 | CASSGQQDTQYF | 27 | 205 | CASSVQDTQYF | 25 | 1287 | CASSLDSNYAEQFF | 24 | 1787 | CASSLGTGYAEQFF | 22 |
| 692 | CASSGQNYAEQFF | 27 | 1198 | CASSSGSAETLYF | 25 | 1334 | CASSLTGNSDYTF | 24 | 1685 | CASGDLGNYAEQFF | 22 |
| 693 | CASSGTASAETLYF | 27 | 1201 | CASSSNYAEQFF | 25 | 304 | CASSIGQYEQYF | 24 | 412 | CASSLTDTQYF | 22 |
| 695 | CASSGTISNERLFF | 27 | 1116 | CASSLTGGAETLYF | 25 | 305 | CASSIGDTQYF | 24 | 413 | CASSFSSYEQYF | 22 |
| 669 | CASGQNTLYF | 27 | 206 | CASSLRGYEQYF | 25 | 1370 | CASSPRAETLYF | 24 | 414 | CASSPDRGEQYF | 22 |
| 115 | CASGEDTQYF | 27 | 1082 | CASSLENSDYTF | 25 | 1268 | CASSGQSQNTLYF | 24 | 1883 | CASSSGNTGQLYF | 22 |
| 662 | CASGDNYNSPLYF | 27 | 207 | CASSLEQYEQYF | 25 | 1284 | CASSLDRDSDYTF | 24 | 1847 | CASSQEGNQDTQYF | 22 |
| 665 | CASGDWGNYAEQFF | 27 | 1079 | CASSLEGDTGQLYF | 25 | 1414 | CASSSGTGNERLFF | 24 | 1885 | CASSSGQSQNTLYF | 22 |
| 657 | CASGDAGSAETLYF | 27 | 208 | CASSLEGGEQYF | 25 | 1289 | CASSLDTNTEVFF | 24 | 1813 | CASSLTPNTEVFF | 22 |
| 654 | CASGDAGGYAEQFF | 27 | 1066 | CASSLAGQNTLYF | 25 | 1293 | CASSLEGTEVFF | 24 | 1870 | CASSRGGQNTLYF | 22 |
| 668 | CASGGSQNTLYF | 27 | 1087 | CASSLGHQDTQYF | 25 | 1294 | CASSLENSGNTLYF | 24 | 1748 | CASSLDGGSAETLYF | 22 |
| 817 | CASSRTGENTLYF | 27 | 209 | CASSLGGDEQYF | 25 | 1248 | CASSDRGNTEVFF | 24 | 1776 | CASSLGLGNYAEQFF | 22 |
| 768 | CASSLTTSAETLYF | 27 | 1085 | CASSLGGGYAEQFF | 25 | 1410 | CASSRTGNYAEQFF | 24 | 1744 | CASSLAGGAETLYF | 22 |
| 714 | CASSLDTNSDYTF | 27 | 1178 | CASSRQSQNTLYF | 25 | 306 | CASSLDSAEQFF | 24 | 1920 | CSAGNSDYTF | 22 |
| 737 | CASSLGNYAEQFF | 27 | 1112 | CASSLSNTEVFF | 25 | 1419 | CASSSQGTEVFF | 24 | 1922 | CSAGSAETLYF | 22 |
| 773 | CASSPDSNERLFF | 27 | 1109 | CASSLSGQNTLYF | 25 | 1278 | CASSLAGNTLYF | 24 | 1909 | CAWSLGGQNTLF | 22 |
| 816 | CASSRQNYAEQFF | 27 | 1110 | CASSLSGSGNTLYF | 25 | 1250 | CASSDRNYAEQFF | 24 | 1910 | CAWSLGNYAEQFF | 22 |
| 805 | CASSRDSNERLFF | 27 | 210 | CASSWDSSYEQYF | 25 | 1377 | CASSQDSGNTLYF | 24 | 1912 | CAWSLGYEQYF | 22 |
| 761 | CASSLSGGQNTLYF | 27 | 211 | CASSLQGGEQYF | 25 | 307 | CASSQDRNTEVFF | 24 | 1857 | CASSRDINQDTQYF | 22 |
| 739 | CASSLGQGAETLYF | 27 | 1075 | CASSLDRTEVFF | 25 | 1307 | CASSLGTGNTGQLYF | 24 | 1834 | CASSPRDSAETLYF | 22 |
| 733 | CASSLGGNYAEQFF | 27 | 212 | CASSLGSYEQYF | 25 | 1367 | CASSPNTEVFF | 24 | 1881 | CASSSGGSQNTLYF | 22 |
| 655 | CASGDAGNTGQLYF | 27 | 213 | CASSLGDSSYEQYF | 25 | 1221 | CASGDDAETLYF | 24 | 415 | CASSLGGGQDTQYF | 22 |
| 775 | CASSPGAGSNERLFF | 27 | 214 | CASSLGVEQYF | 25 | 1265 | CASSGDSAETLYF | 24 | 1725 | CASSFLGNYAEQFF | 22 |
| 116 | CASSRQDTQYF | 27 | 215 | CASSFGGQDTQW | 25 | 1403 | CASSRQTNTEVFF | 24 | 416 | CASGDIYEQYF | 22 |
| 723 | CASSLEGSDYTF | 27 | 1145 | CASSPTGNQDTQW | 25 | 1665 | CAWSLSAETLYF | 23 | 1799 | CASSLQNSGNTLYF | 22 |
| 117 | CASSLGQQDTQYF | 27 | 1133 | CASSPGQNSDYTF | 25 | 1631 | CASSRQGETLYF | 23 | 1792 | CASSLLGENTLYF | 22 |
| 756 | CASSLQGYAEQFF | 27 | 1173 | CASSRLGSQNTLYF | 25 | 1505 | CASSINSAETLYF | 23 | 1707 | CASRSQNTLYF | 22 |
| 747 | CASSLGVYAEQFF | 27 | 1180 | CASSRTASAETLYF | 25 | 308 | CASSEQDTQYF | 23 | 1838 | CASSPTGYAEQFF | 22 |
| 670 | CASKTANQDTQYF | 27 | 1184 | CASSRTGNTGQLYF | 25 | 1484 | CASSDRDSGNTLYF | 23 | 1889 | CASSSLGDQDTQYF | 22 |
| 821 | CASSSGNYAEQFF | 27 | 1081 | CASSLEGTSAETLYF | 25 | 1479 | CASSDGSAETLYF | 23 | 1804 | CASSLRNSGNTLYF | 22 |
| 118 | CASSGGEQYF | 27 | 1083 | CASSLESQNTLYF | 25 | 1482 | CASSDNTGQLYF | 23 | 1816 | CASSLVGSAETLYF | 22 |
| 119 | CASSYQDTQYF | 27 | 1070 | CASSLDNTGQLYF | 25 | 1486 | CASSDRSQNTLYF | 23 | 417 | CASSRDTYEQYF | 22 |
| 766 | CASSLTGGQNTLYF | 27 | 1077 | CASSLEGDQDTQYF | 25 | 309 | CASSGQDTQYF | 23 | 1842 | CASSPWGNYAEQFF | 22 |
| 120 | CASSLYEQYF | 27 | 1076 | CASSLEGANTEVFF | 25 | 310 | CASSLQGEQYF | 23 | 418 | CASSSQGDTQYF | 22 |
| 731 | CASSLGDTEVFF | 27 | 1089 | CASSLGLGAFTLYF | 25 | 311 | CASSLDNSYEQYF | 23 | 419 | CSADQDTQYF | 22 |
| 685 | CASSFDSAETLYF | 27 | 216 | CASSLGVQDTQYF | 25 | 1452 | CASGDNQAPLF | 23 | 1742 | CASSIGAETLYF | 22 |
| 121 | CASSSQDTQYF | 27 | 1205 | CASSTSQNTLYF | 25 | 312 | CASGDSSYEQYF | 23 | 1743 | CASSIGGNYAEQFF | 22 |
| 818 | CASSRTGQNTLYF | 27 | 1182 | CASSRTGGYAEQFF | 25 | 1468 | CASGNQDTQYF | 23 | 1820 | CASSPDNSQNTLYF | 22 |
| 811 | CASSRGSQNTLYF | 27 | 217 | CASSDWGSSYEQYF | 25 | 1460 | CASGDSYNSPLYF | 23 | 1872 | CASSRNYAEQFF | 22 |
| 820 | CASSSGNTLYF | 27 | 1105 | CASSLRQNTEVFF | 25 | 1456 | CASGDRANSDYTF | 23 | 1817 | CASSLVSGNTLYF | 22 |
| 791 | CASSQDWGNYAEQFF | 27 | 218 | CASSLEQDTQYF | 25 | 1445 | CASGDAQSQNTLYF | 23 | 1844 | CASSQDSNQDTQYF | 22 |
| 122 | CASSQDRDTEVFF | 27 | 1062 | CASSLAANTEVFF | 25 | 1444 | CASGDAQDTQYF | 23 | 1845 | CASSQDWGQGTQYF | 22 |
| 690 | CASSGAETLYF | 27 | 1063 | CASSLAGAETLYF | 25 | 1448 | CASGDAYNSPLYF | 23 | 1853 | CASSQGTISNERLFF | 22 |
| 827 | CASSYGQNTLYF | 27 | 1098 | CASSLGVSQNTLYF | 25 | 313 | CASGDASYEQYF | 23 | 1846 | CASSQDWGSAETLYF | 22 |
| 819 | CASSSGGQNTLYF | 27 | 1071 | CASSLDRAGNTLYF | 25 | 1638 | CASSRTGGNTLYF | 23 | 1851 | CASSQGQQNTLYF | 22 |
| 823 | CASSSGTTNTEVFF | 27 | 1121 | CASSNYAEQFF | 25 | 314 | CASSLTGGQDTQYF | 23 | 420 | CASSPLGYEQYF | 22 |
| 825 | CASSSTANSDYTF | 27 | 1061 | CASSINQDTQYF | 25 | 315 | CASSLEGGQDTQYF | 23 | 421 | CASSLRDNYEQYF | 22 |
| 826 | CASSWGSQNTLYF | 27 | 1148 | CASSPTVSQNTLYF | 25 | 1604 | CASSQGANSDYTF | 23 | 1811 | CASSLTGSGNTLYF | 22 |
| 762 | CASSLSSAETLYF | 27 | 1125 | CASSPDWGQDTQYF | 25 | 1660 | CASSWTDSGNTLYF | 23 | 1772 | CASSLGGENTLYF | 22 |
| 726 | CASSLENTLYF | 27 | 1141 | CASSPGTNTEVFF | 25 | 316 | CASSLTGGDTQYF | 23 | 1898 | CASSSWGSQNTLYF | 22 |
| 722 | CASSLEGGQNTLYF | 27 | 1138 | CASSPGTANSDYTF | 25 | 1551 | CASSLQGGTEVFF | 23 | 1758 | CASSLDSNTGQLYF | 22 |
| 703 | CASSLDANSDYTF | 27 | 1126 | CASSPGGAETLYF | 25 | 1562 | CASSLSGTEVFF | 23 | 1755 | CASSLDRANTEVFF | 22 |
| 730 | CASSLGANTGQLYF | 27 | 1166 | CASSRDRNTLYF | 25 | 317 | CASSFTGEDTQYF | 23 | 1749 | CASSLDGNSDYTF | 22 |
| 770 | CASSLVGSQNTLYF | 27 | 1168 | CASSRDSANSDYTF | 25 | 1666 | CGARDNYAEQFF | 23 | 422 | CASSLAYEQYF | 22 |
| 123 | CASSNQDTQYF | 27 | 1188 | CASSRYAEQFF | 25 | 318 | CASSFRDTQYF | 23 | 423 | CASSLGQSYEQYF | 22 |
| 771 | CASSNSAETLYF | 27 | 1174 | CASSRNQDTQYF | 25 | 1633 | CASSRQGYAEQFF | 23 | 1773 | CASSLGGETLYF | 22 |
| 124 | CASSSSSYEQYF | 27 | 1095 | CASSLGTASAETLYF | 25 | 1646 | CASSSGANTEVFF | 23 | 1775 | CASSLGHTEVFF | 22 |
| 753 | CASSLQGNTLYF | 27 | 1094 | CASSLGTANTGQLYF | 25 | 319 | CASSLEAEQFF | 23 | 1779 | CASSLGLNYAEQFF | 22 |
| 755 | CASSLQGQNTLYF | 27 | 1072 | CASSLDRANSIDYTF | 25 | 320 | CASSLGNYEQYF | 23 | 424 | CASSLVDTQYF | 22 |
| 712 | CASSLDRNTEVFF | 27 | 1056 | CASSGSAETLYF | 25 | 1535 | CASSLGRNTEVFF | 23 | 1796 | CASSLNTLYF | 22 |
| 720 | CASSLEANTEVFF | 27 | 1199 | CASSSGTGNTEVFF | 25 | 1523 | CASSLGGGAETLYF | 23 | 1727 | CASSFQANSDYTF | 22 |
| 125 | CASSLEGAEQFF | 27 | 1193 | CASSSGGAETLYF | 25 | 1497 | CASSFSNERLFF | 23 | 1722 | CASSFDNSGNTLYF | 22 |
| 126 | CASSFGQYEQYF | 27 | 1032 | CASRDSNYAEQFF | 25 | 321 | CASSRQGDTQYF | 23 | 1797 | CASSLPGSQNTLYF | 22 |
| 750 | CASSLNSQNTLYF | 27 | 1035 | CASRGQNYAEQFF | 25 | 1601 | CASSQDRGSAETLYF | 23 | 1728 | CASSFQNYAEQFF | 22 |
| 127 | CASSLTGDEQYF | 27 | 219 | CASSPDRDEQYF | 25 | 1599 | CASSQDGSQNTLYF | 23 | 1731 | CASSFTANTEVFF | 22 |
| 740 | CASSLGQGNTEVFF | 27 | 1140 | CASSPGTGYAEQFF | 25 | 1602 | CASSQDSYNSPLYF | 23 | 425 | CASGEGDTQYF | 22 |
| 688 | CASSFQNTLYF | 27 | 1144 | CASSPTGGYAEQFF | 25 | 1480 | CASSDNERLFF | 23 | 1674 | CASGDAGGNQDTQYF | 22 |
| 801 | CASSRDRDAEQFF | 27 | 1123 | CASSPDRNTEVFF | 25 | 1654 | CASSSTGNSDYTF | 23 | 1681 | CASGDGGNTEVFF | 22 |
| 777 | CASSPGLGQDTQYF | 27 | 1134 | CASSPGQNTLYF | 25 | 322 | CASSSTGGYEQYF | 23 | 1678 | CASGDARYEQYF | 22 |
| 815 | CASSRQGQNTLYF | 27 | 1171 | CASSRGNQDTQYF | 25 | 1566 | CASSLTGDTEVFF | 23 | 1854 | CASSQNYAEQFF | 22 |
| 128 | CASSRQGYEQYF | 27 | 1187 | CASSRTTNTEVFF | 25 | 1565 | CASSLTGDSDTYF | 23 | 1759 | CASSLDSSNERLFF | 22 |
| 808 | CASSRDWGNTLYF | 27 | 1181 | CASSRTGGQNTLYF | 25 | 1559 | CASSLRGNSDYTF | 23 | 1690 | CASGDRGNTGQLYF | 22 |
| 718 | CASSLDWGYAEQFF | 27 | 220 | CASSLRGDTQYF | 25 | 1515 | CASSLEAETLYF | 23 | 1793 | CASSLLGGQNTLYF | 22 |
| 707 | CASSLDQNTLYF | 27 | 1069 | CASSLDGYAEQFF | 25 | 323 | CASSLGQSSYEQYF | 23 | 1703 | CASRDNQDTQYF | 22 |
| 734 | CAS SLGGSGNTLYF | 27 | 1091 | CASSLGNSAETLYF | 25 | 324 | CASSLGDSYEQYF | 23 | 1700 | CASGTANTEVFF | 22 |
| 736 | CASSLGGYAEQFF | 27 | 1093 | CASSLGSSQNTLYF | 25 | 1571 | CASSLVGNTGQLYF | 23 | 1671 | CASGDAANSDYTF | 22 |
| 745 | CASSLGTNTEVFF | 27 | 1192 | CASSSGAETLYF | 25 | 1506 | CASSIRGNTEVFF | 23 | 1677 | CASGDAGVSYEQYV | 22 |
| 675 | CASRTSAETLYF | 27 | 221 | CASSLAGGYEQYF | 25 | 1650 | CASSSGTTNSDYTF | 23 | 426 | CASSRTGVYEQYF | 22 |
| 813 | CASSRQGAETLYF | 27 | 1086 | CASSLGGSDYTF | 25 | 1652 | CASSSQGYAEQFF | 23 | 427 | CASSQDRDEQYF | 22 |
| 792 | CASSQGAGNTLYF | 27 | 222 | CASSLGLGYEQYF | 25 | 325 | CASSWDSQDTQYF | 23 | 1715 | CASSDRDQDTQYF | 22 |
| 728 | CASSLETLYF | 27 | 223 | CASSFDAEQFF | 25 | 1564 | CASSLTGANSDYTF | 23 | 428 | CASSEGYEQYF | 22 |
| 129 | CASSSTGYEQYF | 27 | 224 | CASSLREQYF | 25 | 1538 | CASSLGTGNTEVFF | 23 | 1714 | CASSDPGQGTQYF | 22 |
| 725 | CASSLENTEVFF | 27 | 225 | CASSQQGYEQYF | 25 | 326 | CASSPRGQDTQYF | 23 | 1737 | CASSGTGNTLYF | 22 |
| 708 | CASSLDRDTEVFF | 27 | 1152 | CASSQGAETLYF | 25 | 1624 | CASSRDYAEQFF | 23 | 1736 | CASSGTGGNTLYF | 22 |
| 130 | CASSLDGYEYF | 27 | 1150 | CASSQDSYAEQFF | 25 | 327 | CASSLRSSYEQYF | 23 | 1686 | CASGDNNERLFF | 22 |
| 743 | CASSLGTANTEVFF | 27 | 1156 | CASSQGQNYAEQFF | 25 | 1607 | CASSQGNTLYF | 23 | 429 | CASGESSYEQYF | 22 |
| 689 | CASSTSSAETLYF | 27 | 226 | CASSQGNQDTQYF | 25 | 1590 | CASSPQGNSDYTF | 23 | 1917 | CGARQGNTEVFF | 22 |
| 751 | CASSLNTEVFF | 27 | 1136 | CASSPGQTEVFF | 25 | 328 | CASSRLGYEQYF | 23 | 1913 | CGARDNSGNTLYF | 22 |
| 807 | CASSRDSSQNTLYF | 27 | 1130 | CASSPGQGQNTLYF | 25 | 329 | CASSLPGGQDTQYF | 23 | 1730 | CASSFSSGNTLYF | 22 |
| 673 | CASRRDSAETLYT | 27 | 1128 | CASSPGNSDYTF | 25 | 1513 | CASSLDSTEVFF | 23 | 1830 | CASSPGTGNTEVGG | 22 |
| 774 | CASSPDWGSQNTLYF | 27 | 1135 | CASSPGQQNTLYF | 25 | 330 | CASSLEYEQYF | 23 | 1790 | CASSLGTYAEQFF | 22 |
| 798 | CASSRDNQNTLYF | 27 | 1131 | CASSPGQGTEVFF | 25 | 1509 | CASSLDGNQDTQYF | 23 | 1761 | CASSLEETLYF | 22 |
| 804 | CASSRDRNTENTF | 27 | 1162 | CASSRDNNYAEQFF | 25 | 331 | CASSSGSSYEQYF | 23 | 1899 | CASSTGNYAEQFF | 22 |
| 806 | CASSRDSNYAEQFF | 27 | 227 | CASSRDRGDTQYF | 25 | 1622 | CASSRDWNYAEQFF | 23 | 430 | CASSTGNQDTQYF | 22 |
| 700 | CASSLANSGNTLYF | 27 | 1204 | CASSSTGGYAEQFF | 25 | 332 | CASSRGQYEQYF | 23 | 1782 | CASSLGQGNERLFF | 22 |
| 764 | CASSLTGAETLYF | 27 | 1194 | CASSSGGYAEQFF | 25 | 333 | CASSQGEQYF | 23 | 1738 | CASSGTSAETLYF | 22 |
| 715 | CASSLDWGNTGQLYF | 27 | 1117 | CASSLTGNTGQLYF | 25 | 334 | CASSLDGDTQYF | 23 | 1865 | CASSRDTNYAEQFF | 22 |
| 131 | CASSSNQDTQYF | 27 | 228 | CASSLAQDTQYF | 25 | 1510 | CASSLDGNYAEQFF | 23 | 1858 | CASSRDISGNTLYF | 22 |
| 674 | CASRTGGYAEQFF | 27 | 1065 | CASSLAGENTLYF | 25 | 1520 | CASSLGDNQDTQYF | 23 | 1841 | CASSPTNTEVFF | 22 |
| 671 | CASRDNSGNTLYF | 27 | 1051 | CASSTGGSAETLYF | 25 | 1572 | CASSLVGQNTLYF | 23 | 1826 | CASSPGQGNTLYF | 22 |
| 132 | CASSPTGDEQYF | 27 | 1114 | CASSLTGDYNEQFFF | 25 | 1542 | CASSLLGSAETLYF | 23 | 1877 | CASSSDSSGNTLYF | 22 |
| 780 | CASSPGQNTGQLYF | 27 | 1090 | CASSLGLGYAEQFF | 25 | 335 | CASRGQANTEVFF | 23 | 1874 | CASSRQNQAPLF | 22 |
| 785 | CASSPQGSAETLYF | 27 | 1183 | CASSRTGNSDYTF | 25 | 336 | CASSPPGQQDTQYF | 23 | 1873 | CASSRQGDSDYTF | 22 |
| 802 | CASSRDREVFF | 27 | 1149 | CASSQANTGQLYF | 25 | 1592 | CASSPSSQNTLYF | 23 | 1791 | CASSLLGAETLYF | 22 |
| 705 | CASSLDNSQNTLYF | 27 | 229 | CASSRDRQDTQYF | 25 | 1589 | CASSPQGANTEVFF | 23 | 431 | CASSLNSYEQYF | 22 |
| 760 | CASSLRSQNTLYF | 27 | 1040 | CASSDNSGNTLYF | 25 | 337 | CASSPGSSYEQYF | 23 | 1806 | CASSLRQNTLYF | 22 |
| 719 | CASSLDYAEQFF | 27 | 230 | CASSDEDTQYF | 25 | 1574 | CASSPDTEVFF | 23 | 1713 | CASSDNSAETLYF | 22 |
| 824 | CASSSQGANTEVFF | 27 | 1045 | CASSEGQNTLYF | 25 | 1584 | CASSPGQNQAPLF | 23 | 1719 | CASSDTANTEVFF | 22 |
| 729 | CASSLGANTEVFF | 27 | 1037 | CASSDANTEVFF | 25 | 1583 | CASSPGQKNTLYF | 23 | 1699 | CASGSQNTLYF | 22 |
| 735 | CASSLGGTEVFF | 27 | 1039 | CASSDGGNYAEQFF | 25 | 1632 | CASSRQGEVFF | 23 | 1684 | CASGDGTANSDYTF | 22 |
| 133 | CASSLSAEQFF | 27 | 1055 | CASSGQGQNTLYF | 25 | 338 | CASSRDQDTQYF | 23 | 1827 | CASSPGQISNERLI=FF | 22 |
| 687 | CASSFGGQNTL | 27 | 1060 | CASSGTTNTGQLYF | 25 | 1623 | CASSRDWSAETLYF | 23 | 1741 | CASSHNYAEQFF | 22 |
| 706 | CASSLDNTEVEF | 27 | 1058 | CASSGTGNSDYTF | 25 | 339 | CASSRDFYEQYF | 23 | 1810 | CASSLSVNQDTQYF | 22 |
| 134 | CASSLDRGEQYF | 27 | 1196 | CASSSGQGAETLYF | 25 | 1547 | CASSLQGANERLFF | 23 | 1921 | CSAGNTEVFF | 22 |
| 135 | CASSLGYAEQFF | 27 | 1206 | CASSWDNTEVFF | 25 | 1529 | CASSLGQGAGNTLYF | 23 | 1839 | CASSPTISNERLFF | 22 |
| 686 | CASSFDTEVFF | 27 | 231 | CASGDNYEQYF | 25 | 1488 | CASSDTGQYF | 23 | 1814 | CASSLTTNTGQLYF | 22 |
| 136 | CASSSQGYEYF | 27 | 1025 | CASGDRNTEVFF | 25 | 1502 | CASSGTGGQDTQYF | 23 | 432 | CASSYAEQFF | 22 |
| 795 | CASSQTSAETLYF | 27 | 1021 | CASGDPSAETLYF | 25 | 1651 | CASSSQGAGNTLYF | 23 | 1708 | CASSADSAETLYF | 22 |
| 778 | CASSPGNTEVFF | 27 | 1007 | CASGDADEQYF | 25 | 1645 | CASSSDWGQNTLYF | 23 | 1825 | CASSPGLSQNTLYF | 22 |
| 137 | CASSPSYEQYF | 27 | 1012 | CASGDAGGAETLYF | 25 | 340 | CASSSEDTQYF | 23 | 433 | CASSLGTGDTQYF | 22 |
| 138 | CASSPDRYEQYF | 27 | 1019 | CASGDGGQNTLYF | 25 | 1659 | CASSWGDQDTQYF | 23 | 1900 | CASSTFSAETLYF | 22 |
| 139 | CASSPNQDTQYF | 27 | 1029 | CASGGTANTEVFF | 25 | 1472 | CASRSAETLYF | 23 | 1919 | CSADAEQFF | 22 |
| 782 | CASSPGTANTGQLYF | 27 | 232 | CASSPTGGQDTQYF | 25 | 1577 | CASSPGGYAEQFF | 23 | 434 | CSAGQYEQYF | 22 |
| 797 | CASSRDKNTLYF | 27 | 233 | CASSPYEQYF | 25 | 1594 | CASSPTGSAETLYF | 23 | 1705 | CASRQGANTEVFF | 22 |
| 140 | CASSLSSYEQYF | 27 | 1119 | CASSLVSAETLYF | 25 | 1627 | CASSRLGANTGQLYF | 23 | 1859 | CASSRDKNTEVFF | 22 |
| 141 | CASSLAGYEQYF | 27 | 234 | CASSLNAEQFF | 25 | 341 | CASSRDRYEQYF | 23 | 435 | CASSQTGYEQYF | 22 |
| 749 | CASSLNSDYTF | 27 | 1191 | CASSSETLYF | 25 | 1563 | CASSLSNGNTLYF | 23 | 1833 | CASSPQNTEVFF | 22 |
| 142 | CASSLTGGYEQYF | 27 | 235 | CASSFGDTQYF | 25 | 1545 | CASSLNYAEQFF | 23 | 1812 | CASSLTGTEVFF | 22 |
| 757 | CASSLQNSDYTF | 27 | 1015 | CASGDAGNYAEQFF | 25 | 1561 | CASSLSANTEVFF | 23 | 436 | CASSLGLGEDTQYF | 22 |
| 752 | CASSLQGANSDYTF | 27 | 1132 | CASSPGQNQDTQYF | 25 | 1481 | CASSDNSDYTF | 23 | 1884 | CASSSGQKNTLYF | 22 |
| 143 | CASSLDTYEQYF | 27 | 1143 | CASSPLGSQNTLYF | 25 | 342 | CASSEGSSYEQYF | 23 | 1902 | CASSWTANTGQLYF | 22 |
| 711 | CASSLDRNSDYTF | 27 | 236 | CASSLQSSYEQYF | 25 | 1658 | CASSVSAETLYF | 23 | 1754 | CASSLDNNQAPLF | 22 |
| 716 | CASSLDWGQNTLYF | 27 | 237 | CASSPGQDTQYF | 25 | 1556 | CASSLRDNYAEQFF | 23 | 1750 | CASSLDGNTGQLYF | 22 |
| 727 | CASSLEQNSDYTF | 27 | 238 | CASSPGQGNTEVFF | 25 | 343 | CASSLGDAEQFF | 23 | 1745 | CASSLAQQNTEVFF | 22 |
| 144 | CASSLGGSSYEQYF | 27 | 1161 | CASSRDISQNTLYF | 25 | 1493 | CASSFDTGQLYF | 23 | 1698 | CASGPGQGYAEQFF | 22 |
| 769 | CASSLVGNQDTQYF | 27 | 1200 | CASSSNERLFF | 25 | 1540 | CASSLLGNQDTQYF | 23 | 1701 | CASGTGNSDYTF | 22 |
| 812 | CASSRLGNYAEQFF | 27 | 1080 | CASSLEGSSAETLYF | 25 | 1609 | CASSQQGSAETLYF | 23 | 1688 | CASGDPANSDYTF | 22 |
| 814 | CASSRQGNTLYF | 27 | 239 | CASSLDRGYEQYF | 25 | 344 | CASSQDQDTQYF | 23 | 1693 | CASGDTSAETLYF | 22 |
| 682 | CASSDSSGNTLYF | 27 | 1158 | CASSQQGAETLYF | 25 | 1595 | CASSPTINQDTQYF | 23 | 1689 | CASGDRDNYAEQFF | 22 |
| 678 | CASSDANSDYTF | 27 | 240 | CASSLNERLFF | 25 | 1591 | CASSPRGAETLYF | 23 | 1679 | CASGDAWGQDTQYF | 22 |
| 677 | CASSDAGQNTLYF | 27 | 1214 | CSASQNTLYF | 25 | 1585 | CASSPGTANERLFF | 23 | 1683 | CASGDSAETLYF | 22 |
| 679 | CASSDGSQNTLYF | 27 | 1212 | CSAGSQNTLYF | 25 | 345 | CASSPGTGQDTQYF | 23 | 1875 | CASSRTAETLFY | 22 |
| 676 | CASSASAETLYF | 27 | 1213 | CSASAETLYF | 25 | 1579 | CASSPGLGSQNTLYF | 23 | 1863 | CASSRDTSQNTLYF | 22 |
| 696 | CASSGTVSNERLFF | 27 | 241 | CASSLLGGQDTQYF | 25 | 1575 | CASSPGANTGQLYF | 23 | 1789 | CASSLGTTSAETLYF | 22 |
| 694 | CASSGTGGYAEQFF | 27 | 242 | CASSQEDTQYF | 25 | 1576 | CASSPGGSAETLYF | 23 | 1712 | CASSGDTANSDYTF | 22 |
| 145 | CASGYEQYF | 27 | 1146 | CASSPTTSQNTLYF | 25 | 1630 | CASSRQANTGQLYF | 23 | 1843 | CASSQDRANTEVFF | 22 |
| 666 | CASGENTLYF | 27 | 1170 | CASSRGAETLYF | 25 | 1628 | CASSRLGENTLYF | 23 | 1828 | CASSPGQNNQAPLF | 22 |
| 667 | CASGGQNYAEQFF | 27 | 1167 | CASSRDRNYAEQFF | 25 | 1637 | CASSRTASQNTLYF | 23 | 437 | CASSQDYEQYF | 22 |
| 664 | CASGDWGNQDTQYF | 27 | 1068 | CASSLDGNTEVFF | 25 | 1640 | CASSERFNRLYD | 23 | 1695 | CASGETGNTEVFF | 22 |
| 661 | CASGDNTEVFF | 27 | 1099 | CASSLLGANTGQLYF | 25 | 346 | CASSRTGDQDTQFY | 23 | 1882 | CASSSGLGQDTQYF | 22 |
| 658 | CASGDANSDYTF | 27 | 243 | CASSLGLGQDTQYF | 25 | 1613 | CASSRDGNTGQLYF | 23 | 1704 | CASRGQGNTEVFF | 22 |
| 652 | CASGDAGDTQYF | 27 | 1013 | CASGDAGNQDTQYF | 25 | 1620 | CASSRDWGDTQYF | 23 | 438 | CASSFGETLYF | 22 |
| 651 | CASGDAGAETLYF | 27 | 1118 | CASSLTSQNTLYF | 25 | 347 | CASSLQGRDTQYF | 23 | 1733 | CASSGNTLYF | 22 |
| 656 | CASGDAGQNTFVFF | 27 | 244 | CASSLGLQDTQYF | 25 | 348 | CASSWTGEDTQYF | 23 | 1765 | CASSLEGGNTLYF | 22 |
| 653 | CASGDAGGQNTLYF | 27 | 245 | CASSLQGDEQYF | 25 | 349 | CASSWGYEQYF | 23 | 1734 | CASSGQNTGQLYF | 22 |
| 809 | CASSRDWGSQNTLYF | 27 | 1084 | CASSLGGGQNTLYF | 25 | 1544 | CASSLNNYAEQFF | 23 | 1903 | CASSYGAETLYF | 22 |
| 784 | CASSPQGAETLYF | 27 | 1185 | CASSRTGSQNTLYF | 25 | 350 | CASSLRGQDTQFY | 23 | 1709 | CASSAGTABTEVFF | 22 |
| 800 | CASSRDNSQNTLYF | 27 | 1078 | CASSLEGDSDYTF | 25 | 1516 | CASSLEGGNYAEQFF | 23 | 439 | CASSLGTGYEQYF | 22 |
| 748 | CASSLLGGYAEQFF | 27 | 1092 | CASSLGQGTEVFF | 25 | 351 | CASSLEGVEQYF | 23 | 1675 | CASGDAGISNERLFF | 22 |
| 697 | CASSLAETLYF | 27 | 1050 | CASSFGGNYAEQFF | 25 | 1507 | CASSLAGGQNTLYF | 23 | 1768 | CASSLEGTGNTLFY | 22 |
| 742 | CASSLGQYAEQFF | 27 | 1154 | CASSQGNTGQLYF | 25 | 1569 | CASSLTGNQDTQYF | 23 | 1805 | CASSLRNTEVFF | 22 |
| 713 | CASSLDRNTGQLYF | 27 | 1163 | CASSRDNQAPLF | 25 | 1567 | CASSLTGDTGQYLF | 23 | 1720 | CASSEDTEVFF | 22 |
| 721 | CASSLEGAGNTLYF | 27 | 1160 | CASSQQGSGNTLYF | 25 | 1512 | CASSLDSANNTEVFF | 23 | 1871 | CASSRGQGYAEQFF | 22 |
| 717 | CASSLDWGSQNTLYT | 27 | 1124 | CASSPDRSQNTLYF | 25 | 1527 | CASSLGNERLFF | 23 | 1852 | CASSQGTASAETLYF | 22 |
| 754 | CASSLGNYAEQFF | 27 | 246 | CASSPGLGEDTQYF | 25 | 1522 | CASSLGGAGNTLYF | 23 | 1906 | CASSYQANTEVFF | 22 |

In the next step, the literature was searched for additional annotated TCRβ sequences in various models in different strains of mice bearing varying MHC haplotypes. 252 TCRβ sequences that were previously annotated to be associated with defined immune functions were collected from the literature, and compared with the CDR3 dataset. The annotated sequences were associated with four categories of immune reactions: a) Immunity to foreign pathogens; b) Allograft reactions; c) Tumor-related T cells; and d) Autoimmune conditions. Of the 252 annotated CDR3 sequences, 124 sequences were identified that were also present in one or more of the 28 healthy C57BL/6 mice (see Table 11).

The finding of annotated sequences in the dataset of healthy mice, presented herein for the first time (Table 11), highlights a functional difference between the more private TCR sequences, which was found to be associated with all classes of antigens, and the more public sequences, which appear to be associated mainly with autoimmune conditions, allograft reactions and tumor infiltration (FIGS. 6-9). The standard clonal selection paradigm of adaptive immunity would predict that T cells expressing TCRs capable of binding to self-antigens must be deleted during development, most likely in the thymus. Yet, as shown here, a set of autoimmune CDR3 as sequences are commonly shared and even appear to be amplified with high frequency and convergent recombination.

TABLE 11
Annotated CDR3 amino acid sequences present in at least one of the 28 mice tested.

| | | | | SEQ | | | | | | |
|---|---|---|---|---|---|---|---|---|---|---|
| | | H2 | | ID | | | Dominant | Dominant | | |
| Immune Model | Mouse strain | haplotype | Antigen | NO: | CDR3 aa | CR* | V* | J* | Category | Ref. |
| SLE | (NZB X | d/q F1 | Co-culture with | 1926 | CASGDAGAANSDYTF | 1 | V8.2 | J1.2 | Autoimmune | (Adams et al. |
| | SWR)F1 | | syngeneic B cells | 1927 | CASGDAGAAAEQFF | 1 | V8.2 | J2.1 | Autoimmune | 1991) |
| | | | producing anti- | 1928 | CASGDAGAAETLYF | 3 | V8.2 | J2.3 | Autoimmune | |
| | | | DNA | 1929 | CASGDRDYAEQFF | 4 | V8.2 | J2.1 | Autoimmune | |
| | | | autoantibodies. | 1930 | CASSDGQYAEQFF | 7 | V8.1 | J2.1 | Autoimmune | |
| | | | | 1931 | CASSGDRDAEQFF | 4 | V8.3 | J2.1 | Autoimmune | |
| | | | | 1932 | CASSDDRGGGTEVEF | 1 | V8.1 | J1.1 | Autoimmune | |
| | | | | 1933 | CASSQDPGGAETLYF | 3 | V4 | J2.3 | Autoimmune | |
| | | | | 1934 | CASSQGDWGYEQYF | 4 | V1 | J2.7 | Autoimmune | |
| Diabetes | NOD | g7 | Spontaneously | 1925 | CASSLGGNQDTOYF | 53 | V16 | J2.5 | Autoimmune | (Tikochinski |
| | | | reactive 10 p277 | 1935 | CASSSLGGNQDTQYF | 7 | V9 | J2.5 | Autoimmune | et al. 1999) |
| | | | peptide of HSP60 | 1936 | CASRLGNQDTQYF | 8 | V6 | J2.5 | Autoimmune | (Nakano et |
| | | | Spontaneously | 5 | CASGGYEQYF | 9 | V8.2 | J2.7 | Autoimmune | al. 1991) |
| | | | generated islet- | 1924 | CASSLGTTNTGQLYF | 16 | V16 | J2.2 | Autoimmune | |
| | | | reactive T cell | 1937 | CGAGQGGNTLYF | 2 | V15 | J1.3 | Autoimmune | |
| | | | clones | | | | | | | |
| EAE | B10.PL/J | u | Immunized with | 1938 | CASGDAGGGYEQYF | 6 | V8.2 | J2.7 | Autoimmune | (Menezes et |
| | | | MBP peptide | 27 | CASGDAGGSYEQYF | 11 | V8.2 | J2.7 | Autoimmune | al. 2007) |
| | | | Ac1-9 | 30 | CASSGTDQDTQYF | 12 | V8.3 | J2.5 | Autoimmune | |
| | | | | 1939 | CASSLDWDNQDTQYF | 2 | V5.2 | J2.5 | Autoimmune | |
| CIA | DBAI/ILacJ | q | Immunized with | 1940 | CASSQVGNQDTQYF | 9 | V1 | J2.5 | Autoimmune | (Osman et al. |
| | | | bovine collagen | 32 | CASSRTANTGQLYF | 20 | V12 | J2.2 | Autoimmune | 1993) |
| | | | type II | | | | | | | |
| COPD | BALB/cJ | d | Chronic exposure | 1941 | CASRLTSSAETLYF | 1 | V3.1 | J2.3 | Autoimmune | (Motz et al. |
| | | | to cigarette | 1942 | CASRLTSYNSPLYF | 1 | V12 | J1.6 | Autoimmune | 2008) |
| | | | smoke | 1943 | CASSFGDSDYTF | 7 | V7 | J1.2 | Autoimmune | |
| | | | | 21 | CASSLGNSDYTF | 37 | V5.2 | J1.2 | Autoimmune | |
| | | | | 23 | CASSSGNSDYTF | 20 | V10 | J1.2 | Autoimmune | |
| | | | | 22 | CASSSANSDYTF | 18 | V7 | J1.2 | Autoimmune | |
| | | | | 1944 | CASSHRASDYTF | 3 | V4 | J1.2 | Autoimmune | |
| | | | | 1945 | CASSFGGSDYTF | 7 | V16 | J1.2 | Autoimmune | |
| | | | | 1946 | CASSFEGSDYTF | 1 | V1 | J1.2 | Autoimmune | |
| | | | | 1947 | CASGTGGSDYTF | 2 | V8.2 | J1.2 | Autoimmune | |
| | | | | 1948 | CASSGTVNNQAPLF | 2 | V8.3 | J1.5 | Autoimmune | |
| | | | | 1949 | CASSFTNNNQAPLF | 1 | V7 | J1.5 | Autoimmune | |
| | | | | 1950 | CASSFPGNNQAPLF | 1 | V11 | J1.5 | Autoimmune | |
| gp100 | C57BL/6 | b | Cells were sorted | 1951 | CASRDGSYNSPLYF | 1 | V6 | J1.6 | Cancer | (Rizzuto et |
| | | | using tetramers | 1952 | CASSSTGYYAEQFF | 2 | V7 | J2.1 | Cancer | al. 2009) |
| | | | to the Db epitope | 1953 | CASSLSTGYYAEQFF | 1 | V3.1 | J2.1 | Cancer | |
| | | | of gp100 | 1954 | CASSSGGGYAEQFF | 7 | V16 | J2.1 | Cancer | |
| MDM2 | C57BL/6 | b | Immunized with | 26 | CASGDWGYEQYF | 15 | V8.2 | J2.7 | Cancer | (Stanislawski |
| | | | hMDM2(81-88) | | | | | | | et al. 2001) |
| | | | peptide | | | | | | | |
| Glycopeptide | CBA/J | k | Immunized to | 1955 | CASSPGQTYAEQFF | 3 | V5.2 | J2.1 | Cancer | (Jensen et al. |
| | | | hemoglobin (67- | | | | | | | 1999) |
| | | | 76) peptide | | | | | | | |
| | | | substituted with | | | | | | | |
| | | | Thr(α-D- | | | | | | | |
| | | | GalNAc) in | | | | | | | |
| | | | position 72 | | | | | | | |
| P53 | C57BL/6, | b/(HLA)- | p53(264- | 1956 | CASSLSGGGTEVFF | 5 | V3.1 | J1.1 | Cancer | (Kuball et al. |
| | expressing | A*0201 | 272)A2.1 | | | | | | | 2005) |
| | HLA-A | | | | | | | | | |
| Tumor associated | C57BL/6 | HLA- | T cell neoplasms | 29 | CASSHSGNTLYF | 13 | V4 | J1.3 | Cancer | (Raffegerst et |
| | chimeric | DR4/H2Ed | | 1957 | CASSIRDKNTLYT | 5 | V6 | J2.4 | Cancer | al. 2009) |
| | mice | | | 1958 | CASSLGLGVGAETLYF | 2 | V10 | J2.3 | Cancer | |
| | | | | 24 | CASSGTANTEVEF | 16 | V8.3 | J1.1 | Cancer | |
| | | | | 1959 | CASSWDRNYAEQFF | 5 | V10 | J2.1 | Cancer | |
| | | | | 19 | CASSRRPYEQYF | 1 | V9 | J2.7 | Cancer | |
| Tumor infiltrating | C57BL/6 | b | TC-1 tumor cells | 8 | CASSGTGKDTQYF | 10 | V8.1 | J2.5 | Cancer | (Sainz-Perez |
| Tregs | | | expressing | 3 | CASSGTGEDTQYF | 14 | V8.3 | J2.5 | Cancer | et al. 2012) |
| | | | HPV16-E6 and | 2 | CASSGTGQDTQYF | 21 | V83 | J2.5 | Cancer | |
| | | | HPV16-E7 | 1960 | CASSGTGDDTQYF | 1 | V8.1 | J2.5 | Cancer | |
| | | | proteins and | 1961 | CASSGTGRDTQYF | 9 | V8.3 | J2.5 | Cancer | |
| | | | derived from | 16 | CASSGRGQDTQYF | 4 | V6 | J2.5 | Cancer | |
| | | | primary mouse | 1962 | FCASSGGGQDTQYF | 6 | V8.3 | J2.5 | Cancer | |
| | | | lung epithelial | 1963 | CASSGQGQDTQYF | 2 | V8.3 | J2.5 | Cancer | |
| | | | cells. | 1964 | CASSGLGQDTQYF | 6 | V8.1. | J2.5 | Cancer | |
| | | | | 15 | CASTGTGQDTQYF | 7 | V10 | J2.5 | Cancer | |
| | | | | 4 | CASSGLGEDTQYF | 11 | V8.3 | J2.5 | Cancer | |
| | | | | 1965 | CASSGQGEDTQYF | 9 | V8.1 | J2.5 | Cancer | |
| | | | | 1966 | CASSILGDTGQLYF | 3 | V6 | J2.2 | Cancer | |
| | | | | 17 | CASGGAYEQYF | 4 | V5.2 | J2.7 | Cancer | |
| | | | | 1967 | CASSTTGTYEQYF | 1 | V10 | J2.7 | Cancer | |
| | | | | 1968 | CASSDEGGQNTLYF | 7 | V8.1 | J2.4 | Cancer | |
| GVHD | C57BL/6 and | b | C57BL/6 H-2b) | 31 | CASSPGQSNERLFF | 18 | V5.2 | J1.4 | Allograft | (Rangarajan |
| | BALB/cJ | | anti BALB/cJ | 1969 | CASSIWGNYAEQFF | 8 | V6 | J2.1 | Allograft | et al. 2012) |
| | | | | 1970 | CASSDAREPQYF | 1 | V8.3 | J2.5 | Allograft | |
| | | | | 1971 | CASSGRNTGQLYF | 9 | V8.3 | J2.2 | Allograft | |
| | | | | 1972 | CGASDSSGNTLYF | 2 | V15 | J1.3 | Allograft | |
| | | | | 34 | CASSLEGDTEVFF | 19 | V11 | J1.1 | Allograft | |
| | | | | 1973 | CASKTDGNTLYF | 1 | V3.1 | J1.3 | Allograft | |
| | | | | 1974 | CASSIGTGGYAEQFF | 6 | V6 | J2.1 | Allograft | |
| | | | | 1975 | CASSDGWGQNTLYF | 2 | V8.1 | J2.4 | Allograft | |
| | | | | 1976 | CASSDWGGYEQYF | 9 | V8.3 | J2.7 | Allograft | |
| | | | | 1977 | CASSQEGGNTEVFF | 8 | V1 | J1.1 | Allograft | |
| | | | | 1978 | CASSDAGNYEQYF | 5 | V8.1 | J2.7 | Allograft | |
| | | | | 1979 | CASSAGGADTQYF | 1 | V8.3 | J2.5 | Allograft | |
| | | | | 1980 | CASSPGQGDTGQLYF | 6 | VI9 | J2.2 | Allograft | |
| | | | | 1981 | CASSSTWGNYAEQFF | 1 | V10 | J2.1 | Allograft | |
| | | | | 1982 | CGARESSYEQYF | 1 | V15 | J2.7 | Allograft | |
| | | | | 1983 | CGARSSQNTLYF | 7 | V15 | J2.4 | Allograft | |
| Minor | C.B10-H2^{b} | b | C57BL/10SnJ | 20 | CASSLGGQNTLYF | 105 | V16 | J2.4 | Allograft | (Johnston et |
| histoincompatability | (BALB.B), | | (B10) | | | | | | | al. 1997) |
| | C57BL/10SnJ | | C57BL/10SnJ | 1984 | CASSQDWGGSYEQYF | 3 | V1 | J2.7 | Allograft | |
| | (B10), | | (B10) | | | | | | | |
| | B10.129-H4^{b} | | C57BL/6ByJ | 12 | CASSFGTGYEQYF | 6 | V13 | J2.7 | Allograft | |
| | (21 M), and | | (B6) | | | | | | | |
| | C57BL/6ByJ | | C57BL/6ByJ | 25 | CASSGQGNYAEQFF | 17 | V8.3 | J2.1 | Allograft | |
| | (B6) mice. | | (B6) | | | | | | | |
| | | | C57BL/6ByJ | 1985 | CASSFWGGDAEQFF | 2 | V12 | J2.1 | Allograft | |
| | | | (B6) | | | | | | | |
| Schistosoma | CBA/J mice | k | Immunized with | 1986 | CASSQGTGGNTGQLYF | 4 | V1 | J2.2 | Pathogens | (Hogan et al. |
| mansoni | and a TCR | | S. mansoni | 1987 | CASSQDANTGQLYF | 3 | V4 | J2.2 | Pathogens | 2002) |
| | transgenic | | | | | | | | | |
| | strain. | | | | | | | | | |
| Trypanosoma cruzi | BALB/c | d | Immunized with | 1988 | CASSITDTNTEVFF | 1 | V6 | J1.1 | Pathogens | (Hiyane et al. |
| | | | recombinant | | | | | | | 2006) |
| | | | transsialidase, an | | | | | | | |
| | | | immunodominant | | | | | | | |
| | | | T. cruzi antigen. | | | | | | | |
| Histoplasma | C57BL/6 | b | Immunized with | 1989 | CASSQDYRANTEVFF | 2 | V1 | J1.1 | Pathogens | (Scheckelhoff |
| capsulatum | | | H. capsulatum | 1990 | CASSQPGQSNERLFF | 2 | V4 | J1.4 | Pathogens | and Deepe |
| | | | strain G217B | 1991 | CASSQDGTANERLFF | 2 | V4 | J1.4 | Pathogens | 2006) |
| | | | | 1992 | CASSQVGYNSPLYF | 3 | V1 | J1.6 | Pathogens | |
| | | | | 1993 | CASSQLGQNYAEQFF | 1 | V4 | J2.1 | Pathogens | |
| | | | | 1994 | CASSQEMGENTLYF | 1 | V4 | J2.4 | Pathogens | |
| | | | | 1995 | CASSQQGYNSPLYF | 5 | V1 | J1.6 | Pathogens | |
| P. berghei | BALB/c mice | b | Immunized with | 7 | CASSSRYEQYF | 15 | V7 | J2.7 | Pathogens | (Casanova et |
| | | | P. berghei | 1996 | CASSVTGSNTEVFF | 1 | V8.3 | J1.1 | Pathogens | al. 1991) |
| | | | sporozoite | 1997 | CASSPTGRNTEVFF | 6 | V19 | J1.1 | Pathogens | |
| | | | | 1998 | CASGDGNQAPLF | 4 | V8.2 | J1.5 | Pathogens | |
| | | | | 1999 | CASSFRDRGNYAEQFF | 1 | V7 | J2.1 | Pathogens | |
| Influenza | C57BL/J | b | Immunized with | 2000 | CASSSGGSNTGQLYF | 2 | V16 | J2.2 | Pathogens | (Kedzierska |
| | | | HKx31 (H3N2) | 2001 | CASSQSGGSNTGQLYF | 1 | V4 | J2.2 | Pathogens | et al. 2006) |
| | | | influenza A virus | 2002 | CASGGANTGQLYF | 3 | V8.2 | J2.2 | Pathogens | |
| | | | | 2003 | CASGGGNTGQLYF | 3 | V8.2 | J2.2 | Pathogens | |
| | | | | 2004 | CASSRGGANTGQLYF | 2 | V9 | J2.2 | Pathogens | |
| | | | | 2005 | CASSRGGGNTGQLYF | 1 | V9 | J2.2 | Pathogens | |
| | | | | 2006 | CAWSLKGGANTGQLYF | 1 | V14 | J2.2 | Pathogens | |
| | | | | 2007 | CASSARTANTEVFF | 1 | V8.3 | J1.1 | Pathogens | |
| Vesicular stomatitis | F2 cross of | b | Immunized with | 18 | CASSFVGSYEQYF | 1 | V12 | J2.7 | Pathogens | (Wang et al. |
| virus (VSV) | CBA and | | a peptide of the | 2008 | CASSFHISYNSPLYF | 1 | V13 | J1.6 | Pathogens | 1998) |
| | C57BL/6 | | nucleoprotein, of | 2009 | CASSFGVNSDYTF | 1 | V10 | J1.2 | Pathogens | |
| | bearing a | | VSV. | 14 | CASSLGVGDTQYF | 6 | V12 | J2.5 | Pathogens | |
| | TCR α chain | | | 9 | CASSFGVSYEQYF | 4 | V13 | J2.7 | Pathogens | |
| | (Vα2) | | | 2010 | CASSLGTGGKQFF | 1 | V13 | J2.1 | Pathogens | |
| | transgene. | | | 11 | CASSPGTGVEQYF | 10 | V12 | J2.7 | Pathogens | |
| | | | | 2011 | CASSPGTGQAPLF | 4 | V12 | J1.5 | Pathogens | |
| Lymphocytic | P14 TcR a- | | Immunized with | 28 | CASSDGANTEVVV | 9 | V8.3 | J1.1 | Pathogens | (Brandie et |
| choriomeningitis | transgenic | | the LCMV-WE | 2012 | CASSDQANTEVFF | 5 | V8.3 | J1.1 | Pathogens | al, 1991) |
| virus (LCMV) | mice were | | variant peptides. | 33 | CASSDSANTEVFF | 10 | V8.1 | J1.1 | Pathogens | |
| | generated by | | | 2013 | CASSDAGANTEVFF | 12 | V8.3 | J1.1 | Pathogens | |
| | injection of | | | 2014 | CASSDNANSDYTF | 2 | V8.3 | J1.2 | Pathogens | |
| | the P14 TcR | | | 2015 | CASSDAGHSPLYF | 1 | V8.3 | J1.6 | Pathogens | |
| | a construct | | | | | | | | | |
| | which | | | | | | | | | |
| | contained the | | | | | | | | | |
| | H-2Kb | | | | | | | | | |
| | promoter, | | | | | | | | | |
| | P14 TcR a | | | | | | | | | |
| | cDNA, P- | | | | | | | | | |
| | globin | | | | | | | | | |
| | fragment and | | | | | | | | | |
| | IgH enhancer. | | | | | | | | | |
| | P14 TcR P- | | | | | | | | | |
| | transgenic | | | | | | | | | |
| | mice. | | | | | | | | | |
| Murine leukemia | (BALB/c x | b, b, bc, b | Immunized with | 2016 | CASSQDWGFQDTQYF | 2 | V1 | J2.5 | Pathogens | (Iwashiro et |
| virus (MuLV) | B6)(CB6)F1 | | fragments of the | 2017 | CAWSRTGGNSDYTF | 3 | V14 | J1.2 | Pathogens | al. 1993) |
| | | | F-MuLV env | | | | | | | |
| | | | gene | | | | | | | |

### Example 2: Antibodies to a Public CDR3 Peptide Sequence Unleash an Autoimmune Effector Response

Without being bound by theory, since healthy C57BL/6 mice harbored public CDR3 TCR clonotypes associated with self-reactivity and tumor reactivity, it was thought that these sets of T cells probably served as regulatory T cells, rather than directly as effector T cells. In the case of an autoimmune disease such as Type 1 Diabetes, the public C9 CDR3 clonotype functioned to prevent or down-regulate the disease—this has been shown to be case both in NOD mice and humans with Type 1 Diabetes: a DiaPep277 peptide works in both NOD mice and in human phase 3 clinical trials to arrest beta-cell destruction by activating C9 regulatory T cells (Schloot N C, Cohen I R., Clin Immunol. 149(3):307-16, 2013). Consequently, targeting the C9 CDR3 T cell set by a specific antibody should unleash more severe autoimmune diabetes. To this end, NOD mice were injected with antibodies against the CDR3 peptide of a relatively public TCR clone from the dataset, which was previously associated with type 1 diabetes. The graphs of FIG. 10 show incidence of diabetic mice in a group of male (left) and female (right) mice. Gray: a single injection of anti-CDR3 antibodies; Black: a single injection of control antibodies. The anti-CDR3 antibody increased disease incidence in both males and females. Male NOD mice, which have a very low spontaneous incidence of diabetes, develop a high incidence of severe diabetes subsequent to a single administration of rabbit antibodies raised against the C9 CDR3 peptide; females respond with accelerated, more severe disease too. Similarly, animal models for other autoimmune allograft diseases and disorders, such as Experimental autoimmune encephalomyelitis (EAE) for multiple sclerosis (described for example in Abramsky et. al., J Neuroimmunol, 1982, 2, 1 and Bolton et al., J Neurol Sci., 1982, 56, 147); Systemic Lupus Erythematosus (SLE) model (described for example in U.S. Pat. No. 6,613,536); and skin graft model for allograft rejection (described for example in Rosenberg AS1 and Singer A, Annu Rev Immunol. 1992; 10:333-58), are used to test the efficacy of the peptides for clinical applications.

### Example 3: Inducing Tumor-Specific “Autoimmune” Response

Tumor immunotherapy and autoimmune disease immunotherapy are two sides of the same coin: in autoimmune disease it is desired to activate natural regulatory T clonotypes by activating a disease-associated public CDR3 clonotype using its specific antigen. In tumor immunotherapy, in contrast, it is desired to inactivate the natural regulatory, public CDR3 clonotypes mobilized by the tumor for its own protection.

Consequently, targeting the C9 CDR3 T cell set by a specific antibody may inhibit the growth of tumors. To this end, C57BL/6 mice were injected with syngeneic mouse Lewis lung carcinoma cell line and treated with antibodies against the C9-CDR3 peptide or with un-immunized serum control. As clearly shown in FIG. 11, 1 month following tumor injection the mean number of lung nodules in mice that received two injections of the anti-C9-CDR3 antibody was significantly lower (p=0.026) than in the control group. These results indicate that the anti-C9-CDR3 antibody can inhibit the growth of a syngeneic tumor in a model of disseminated tumor growth.

While the present invention has been particularly described, persons skilled in the art will appreciate that many variations and modifications can be made. Therefore, the invention is not to be construed as restricted to the particularly described embodiments, rather the scope, spirit and concept of the invention will be more readily understood by reference to the claims which follow.

1. An isolated agent capable of at least one of:
(i) binding a TCR presented on a T cell;
(ii) competing with binding of a TCR presented on a T cell to a target of said T cell;
(iii) eliciting a specific immune-response of a T cell; and
(iv) eliciting a specific immune-response against a T cell;
wherein said T cell is expressing a TCR-CDR3 sequence comprising an amino acid sequence selected from the group consisting of SEQ ID NOs of Table 8, wherein when said agent is a peptide it is selected from the group consisting of SEQ ID NOs of Table 7.

2-3. (canceled)

4. A method of treating a disease associated with said T cell, the method comprising administering to a subject in need thereof an effective amount of:
(i) the isolated agent of claim 1;
or
(ii) an isolated peptide of no more than 20 amino acids comprising an amino acid sequence having a CDR3 sequence of a TCR on a T cell, said CDR3 sequence being selected from the group consisting of SEQ ID NOs of Table 8,
thereby treating the disease associated with said T cell.

5. The isolated agent of claim 1, wherein said agent is selected from the group consisting of antibody, T cell, peptide and polynucleotide.

6. An isolated antibody comprising an antigen recognition domain capable of specifically binding SEQ ID NO: 1 of a TCR presented on a T cell.

7. (canceled)

8. A method of treating a disease associated with a T cell expressing a TCR-CDR3 segment comprising an amino acid sequence of SEQ ID NO: 1 in a subject in need thereof, the method comprising administering to the subject a therapeutically effective amount of the isolated antibody of claim 6, thereby treating the disease associated with a T cell expressing said TCR-CDR3 segment comprising an amino acid sequence of SEQ ID NO: 1 in the subject.

9. A pharmaceutical composition comprising as an active ingredient the isolated agent claim 1 and a pharmaceutically acceptable carrier or diluent.

10. The pharmaceutical composition of claim 9 further comprising an adjuvant or a delivery system.

11. The isolated agent of claim 1, wherein said T cell is a regulatory T cell.

12. The isolated agent of claim 11, wherein said T cell is an effector T cell.

13. The method of claim 4, wherein said disease is an autoimmune disease, graft rejection disease, cancer or a pathogenic disease.

14-19. (canceled)

20. The isolated agent of claim 1, wherein said CDR3 sequence and/or said peptide amino acid sequence is selected from the group consisting of the sequences in Table 2.

21. (canceled)

22. The isolated agent of claim 1, wherein said CDR3 sequence and/or said peptide amino acid sequence is selected from the group consisting of the sequences in Table 3.

23. (canceled)

24. The isolated agent of claim 1, wherein said CDR3 sequence and/or said peptide amino acid sequence is selected from the group consisting of the sequences in Table 5.

25. (canceled)

26. The isolated agent of claim 1, wherein said CDR3 sequence is selected from the group consisting of the sequences in Table 7.

27-32. (canceled)

33. The method of claim 4, wherein said peptide amino acid sequence is selected from the group consisting of SEQ ID NOs of Table 7.

34. (canceled)

35. An isolated polynucleotide comprising a nucleic acid sequence encoding the agent of claim 1.

36. An isolated polynucleotide comprising a nucleic acid sequence encoding the antibody of claim 6.

37. A multimer of the isolated peptide of claim 5.

38-39. (canceled)

40. A fusion protein comprising at least one of the isolated peptides of claim 5.

41. The isolated peptide of claim 5, wherein said peptide is attached to a non-proteinaceous moiety.

42-48. (canceled)
